SEQUENCE LISTING (7 sequences)

SEQ ID NO: 1 - protein, 360 aa, Homo sapiens
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 2 - protein, 17 aa, Homo sapiens
  MISC_FEATURE (2)..(16): X is any amino acid
IIXXKRXXREXXLLXXM

SEQ ID NO: 3 - protein, 6 aa, Homo sapiens
DIIHRD

SEQ ID NO: 4 - protein, 10 aa, Homo sapiens
DFGLARHTDD

SEQ ID NO: 5 - protein, 12 aa, Homo sapiens
EMTGYVATRWYR

SEQ ID NO: 6 - protein, 4 aa, Homo sapiens
WMHY

SEQ ID NO: 7 - protein, 4 aa, Homo sapiens
YGSV

(19) United States
(12) Patent Application Publication
(10) Pub. No.: US 2007/0191336 A1
(43) Pub. Date: Aug. 16, 2007

(54) Anti-inflammatory medicaments

(76) Inventors: Daniel L. Flynn, Lawrence, KS (US); Peter A. Petillo, Arlington, MA (US)

Correspondence Address: HOVEY WILLIAMS LLP, 2405 GRAND BLVD., SUITE 400, KANSAS CITY, MO, 64108, US

(21) Appl. No.: 11/022,395

(22) Filed: Dec. 23, 2004

Related U.S. Application Data
(63) Continuation-in-part of application No. 10/886,329, filed on Jul. 6, 2004.
(63) Continuation-in-part of application No. 10/746,460, filed on Dec. 24, 2003, now Pat. No. 7,144,911.

(51) Int. Cl. A61K 31/553; A61K 31/5377; A61K 31/4965; A61K 31/495; A61K 31/426

(52) U.S. Cl. 514/211.01; 514/235.2; 514/255.01; 514/302; 514/303; 514/242; 514/241; 514/365; 514/255.02; 514/389; 514/394; 514/397; 514/406; 514/317

(57) ABSTRACT

Novel compounds and methods of using those compounds for the treatment of inflammatory conditions are provided. In a preferred embodiment, modulation of the activation state of p38 kinase protein comprises the step of contacting the kinase protein with the novel compounds.

### RELATED APPLICATIONS

This application is a continuation-in-part of application Ser. No. 10/746,460 filed Dec. 24, 2003 and application Ser. No. 10/886,329 filed Jul. 6, 2004. This prior application is incorporated by reference herein. This application also claims the benefit of provisional application entitled Enzyme Modulators for treatment of inflammatory, autoimmune, cardiovascular, and immunological diseases, filed ______. All of the foregoing applications are incorporated by reference herein.

### BACKGROUND OF THE INVENTION

1. Field of the Invention

The present invention relates to novel compounds and methods of using those compounds to treat anti-inflammatory diseases.

2. Description of the Prior Art

Basic research has recently provided the life sciences community with an unprecedented volume of information on the human genetic code and the proteins that are produced by it. In 2001, the complete sequence of the human genome was reported (Lander, E. S. et al. Initial sequencing and analysis of the human genome. Nature (2001) 409:860; Venter, J. C. et al. The sequence of the human genome. Science (2001) 291:1304). Increasingly, the global research community is now classifying the 50,000+ proteins that are encoded by this genetic sequence, and more importantly, it is attempting to identify those proteins that are causative of major, under-treated human diseases.

Despite the wealth of information that the human genome and its proteins are providing, particularly in the area of conformational control of protein function, the methodology and strategy by which the pharmaceutical industry sets about to develop small molecule therapeutics has not significantly advanced beyond using native protein active sites for binding to small molecule therapeutic agents. These native active sites are normally used by proteins to perform essential cellular functions by binding to and processing natural substrates or tranducing signals from natural ligands. Because these native pockets are used broadly by many other proteins within protein families, drugs which interact with them are often plagued by lack of selectivity and, as a consequence, insufficient therapeutic windows to achieve maximum efficacy. Side effects and toxicities are revealed in such small molecules, either during preclinical discovery, clinical trials, or later in the marketplace. Side effects and toxicities continue to be a major reason for the high attrition rate seen within the drug development process. For the kinase protein family of proteins, interactions at these native active sites have been recently reviewed: see J. Dumas, Protein Kinase Inhibitors: Emerging Pharmacophores 1997-2001, Expert Opinion on Therapeutic Patents (2001) 11: 405-429; J. Dumas, Editor, New challenges in Protein Kinase Inhibition, in Current Topics in Medicinal Chemistry (2002) 2: issue 9.

It is known that proteins are flexible, and this flexibility has been reported and utilized with the discovery of the small molecules which bind to alternative, flexible active sites with proteins. For review of this topic, see Teague, Nature Reviews/Drug Discovery, Vol. 2, pp. 527-541 (2003). See also, Wu et al., Structure, Vol. 11, pp. 399-410 (2003). However these reports focus on small molecules which bind only to proteins at the protein natural active sites. Peng et al., Bio. Organic and Medicinal Chemistry Ltrs., Vol. 13, pp. 3693-3699 (2003), and Schindler, et al., Science, Vol. 289, p. 1938 (2000) describe inhibitors of abl kinase. These inhibitors are identified in WO Publication No. 2002/034727. This class of inhibitors binds to the ATP active site while also binding in a mode that induces movement of the kinase catalytic loop. Pargellis et al., Nature Structural Biology, Vol. 9, p. 268 (2002) reported inhibitors p38 alpha-kinase also disclosed in WO Publication No. 00/43384 and Regan et al., J. Medicinal Chemistry, Vol. 45, pp. 2994-3008 (2002). This class of inhibitors also interacts with the kinase at the ATP active site involving a concomitant movement of the kinase activation loop.

More recently, it has been disclosed that kinases utilize activation loops and kinase domain regulatory pockets to control their state of catalytic activity. This has been recently reviewed (see, e.g., M. Huse and J. Kuriyan, Cell (2002) 109:275).

### SUMMARY OF THE INVENTION

The present invention is broadly concerned with new compounds for use in treating anti-inflammatory conditions and methods of treating such conditions. In more detail, the inventive compounds have the formula 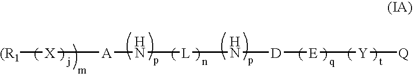
wherein:
R¹ is selected from the group consisting of aryls (preferably C₆-C₁₈, and more preferably C₆-C₁₂) and heteroaryls;
each X and Y is individually selected from the group consisting of —O—, —S—, —NR₆—, —NR₆SO₂—, —NR₆CO—, alkynyls (preferably C₁-C₁₈, and more preferably C₁-C₁₂), alkenyls (preferably C₁-C₁₈, and more preferably C₁-C₁₂), alkylenes (preferably C₁-C₁₈, and more preferably C₁-C₁₂), —O(CH₂)ₕ—, and —NR₆(CH₂)ₕ—, where each h is individually selected from the group consisting of 1, 2, 3, or 4, and where for each of alkylenes (preferably C₁-C₁₈, and more preferably C₁-C₁₂), —O(CH₂)ₕ—, and —NR₆(CH₂)ₕ—, one of the methylene groups present therein may be optionally double-bonded to a side-chain oxo group except that where —O(CH₂)ₕ— the introduction of the side-chain oxo group does not form an ester moiety;
A is selected from the group consisting of aromatic (preferably C₆-C₁₈, and more preferably C₆-C₁₂), monocycloheterocyclic, and bicycloheterocyclic rings;
D is phenyl or a five- or six-membered heterocyclic ring selected from the group consisting of pyrazolyl, pyrrolyl, imidazolyl, oxazolyl, thiazolyl, furyl, oxadiazolyl, thiadiazolyl, thienyl, pyridyl, and pyrimidyl;
E is selected from the group consisting of phenyl, pyridinyl, and pyrimidinyl;
L is selected from the group consisting of —C(O)— and —S(O)₂—;

j is 0 or 1;

m is 0 or 1;

n is 0 or 1;

p is 0 or 1;

q is 0 or 1;

t is 0 or 1;

Q is selected from the group consisting of 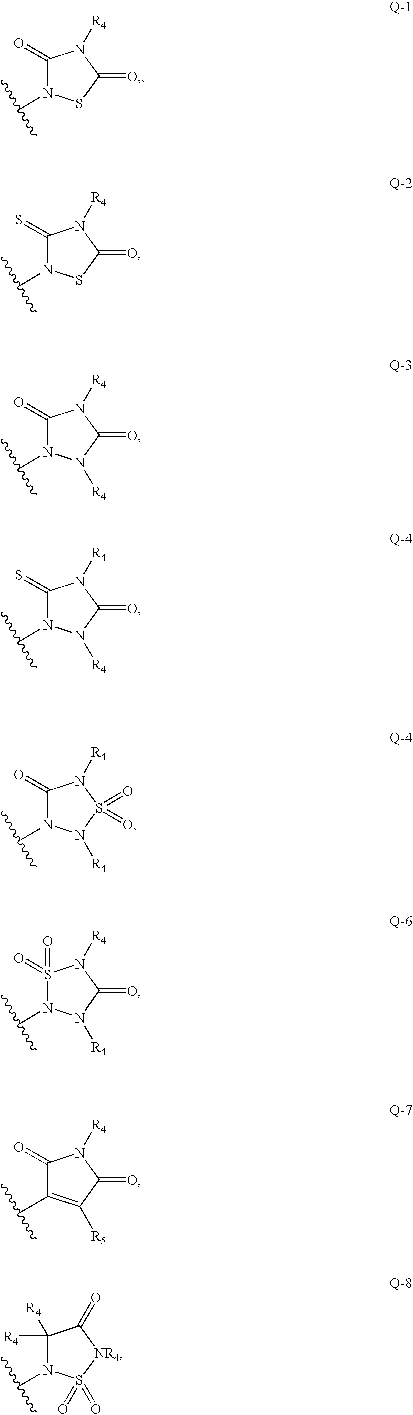 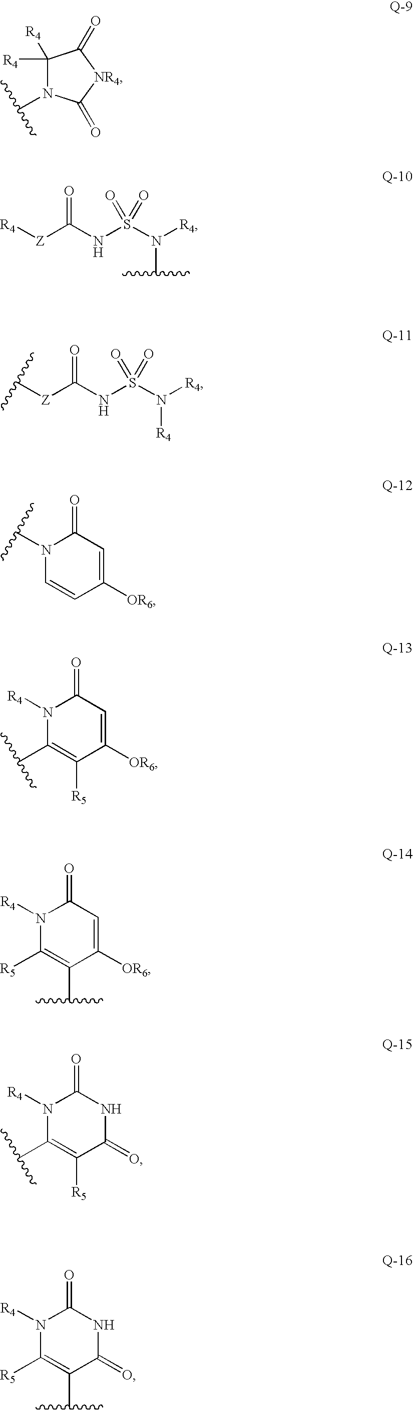 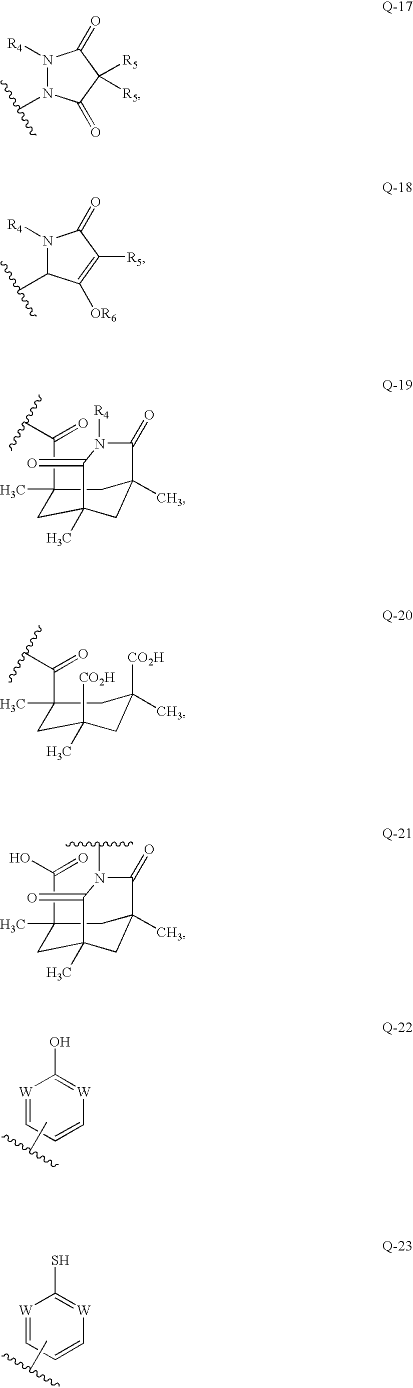 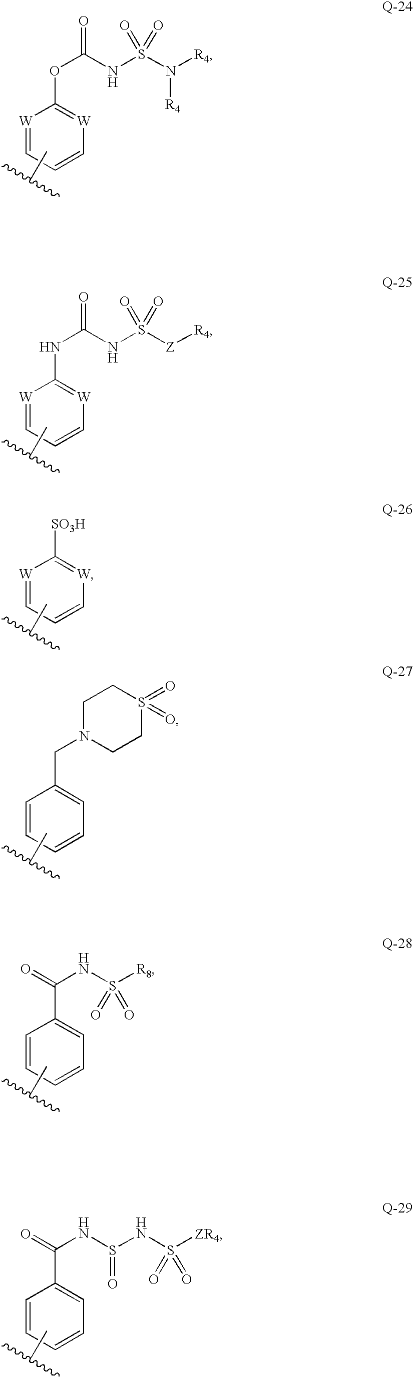 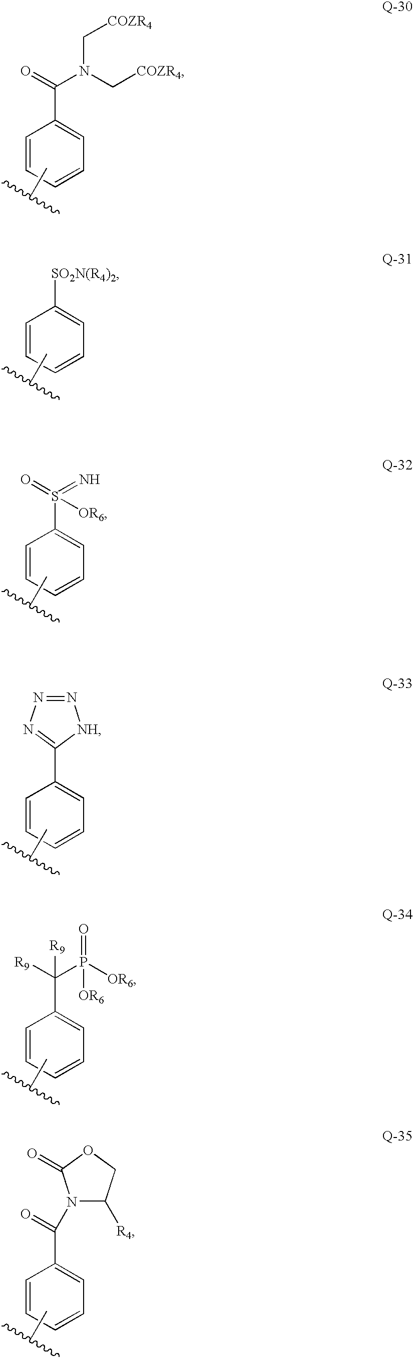 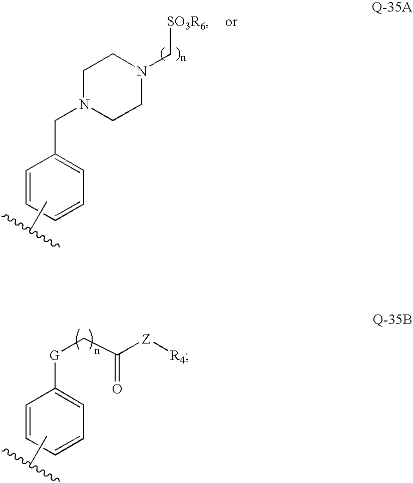
each R₄ group is individually selected from the group consisting of —H, alkyls (preferably C₁-C₁₈, and more preferably C₁-C₁₂), aminoalkyls (preferably C₁-C₁₈, and more preferably C₁-C₁₂), alkoxyalkyls (preferably C₁-C₁₈, and more preferably C₁-C₁₂), aryls (preferably C₆-C₁₈, and more preferably C₆-C₁₂), aralkyls (preferably C₆-C₁₈, and more preferably C₆-C₁₂ and preferably C₁-C₁₈, and more preferably C₁-C₁₂), heterocyclyls, and heterocyclylalkyls except when the R₄ substituent places a heteroatom on an alpha-carbon directly attached to a ring nitrogen on Q;
when two R₄ groups are bonded with the same atom, the two R₄ groups optionally form an alicyclic or heterocyclic 4-7 membered ring;
each R₅ is individually selected from the group consisting of —H, alkyls (preferably C₁-C₁₈, and more preferably C₁-C₁₂), aryls (preferably C₆-C₁₈, and more preferably C₆-C₁₂), heterocyclyls, alkylaminos (preferably C₁-C₁₈, and more preferably C₁-C₁₂), arylaminos (preferably C₆-C₁₈, and more preferably C₆-C₁₂), cycloalkylaminos (preferably C₁-C₁₈, and more preferably C₁-C₁₂), heterocyclylaminos, hydroxys, alkoxys (preferably C₁-C₁₈, and more preferably C₁-C₁₂), aryloxys (preferably C₆-C₁₈, and more preferably C₆-C₁₂), alkylthios (preferably C₁-C₁₈, and more preferably C₁-C₁₂), arylthios (preferably C₆-C₁₈, and more preferably C₆-C₁₂), cyanos, halogens, perfluoroalkyls (preferably C₁-C₁₈, and more preferably C₁-C₁₂), alkylcarbonyls (preferably C₁-C₁₈, and more preferably C₁-C₁₂), and nitros;
each R₆ is individually selected from the group consisting of —H, alkyls (preferably C₁-C₁₈, and more preferably C₁-C₁₂), allyls, and O-trimethylsilylethyl;
   each R₈ is individually selected from the group consisting of alkyls (preferably C₁-C₁₈, and more preferably C₁-C₁₂), phenyl, naphthyl, aralkyls (wherein the aryl is preferably C₆-C₁₈, and more preferably C₆-C₁₂, and wherein alkyl is preferably C₁-C₁₈, and more preferably C₁-C₁₂), heterocyclyls, and heterocyclylalkyls (wherein the alkyl is preferably C₁-C₁₈, and more preferably C₁-C₁₂);
each R₉ group is individually selected from the group consisting of —H, —F, and alkyls (preferably C₁-C₁₈, and more preferably C₁-C₁₂), wherein when two R₉ groups are geminal alkyl groups, said geminal alkyl groups may be cyclized to form a 3-6 membered ring;
   G is alkylene (preferably C₁-C₈, and more preferably C₁-C₄), N(R₆), O;
each Z is individually selected from the group consisting of —O— and —N(R₄)—; and
each ring of formula (IA) optionally includes one or more of R₇, where R₇ is a noninterfering substituent individually selected from the group consisting of —H, alkyls (preferably C₁-C₁₈, and more preferably C₁-C₁₂), aryls (preferably C₆-C₁₈, and more preferably C₆-C₁₂), heterocyclyls, alkylaminos (preferably C₁-C₁₈, and more preferably C₁-C₁₂), arylaminos (preferably C₆-C₁₈, and more preferably C₆-C₁₂), cycloalkylaminos (preferably C₁-C₁₈, and more preferably C₁-C₁₂), heterocyclylaminos, hydroxys, alkoxys (preferably C₁-C₁₈, and more preferably C₁-C₁₂), aryloxys (preferably C₆-C₁₈, and more preferably C₆-C₁₂), alkylthios (preferably C₁-C₁₈, and more preferably C₁-C₁₂), arthylthios, cyanos, halogens, nitrilos, nitros, alkylsulfinyls (preferably C₁-C₁₈, and more preferably C₁-C₁₂), alkylsulfonyls (preferably C₁-C₁₈, and more preferably, C₁-C₁₂), aminosulfonyls, and perfluoroalkyls (preferably C₁-C₁₈, and more preferably C₁-C₁₂).

In one preferred embodiment, the compound has the structure of formula (I) except that:
when Q is Q-3 or Q-4, then the compound of formula (I) is not 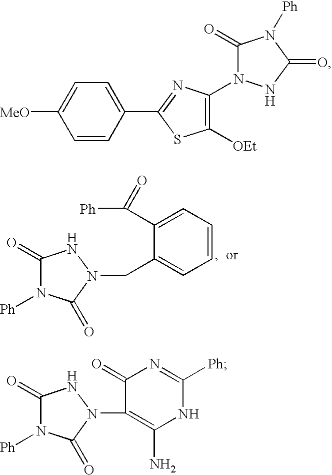
when Q is Q-7, q is 0, and R₅ and D are phenyl, then A is not phenyl, oxazolyl, pyridyl, pyrimidyl, pyrazolyl, or imidazolyl;
when Q is Q-7, R₅ is —OH, Y is —O—, —S—, or —CO—, m is 0, n is 0, p is 0, and A is phenyl, pyridyl, or thiazolyl, then D is not thienyl, thiazolyl, or phenyl;
when Q is Q-7, R₅ is —OH, m is 0, n is 0, p is 0, t is 0, and A is phenyl, pyridyl, or thiazolyl, then D is not thienyl, thiazolyl, or phenyl;
when Q is Q-7, then the compound of formula (I) is not 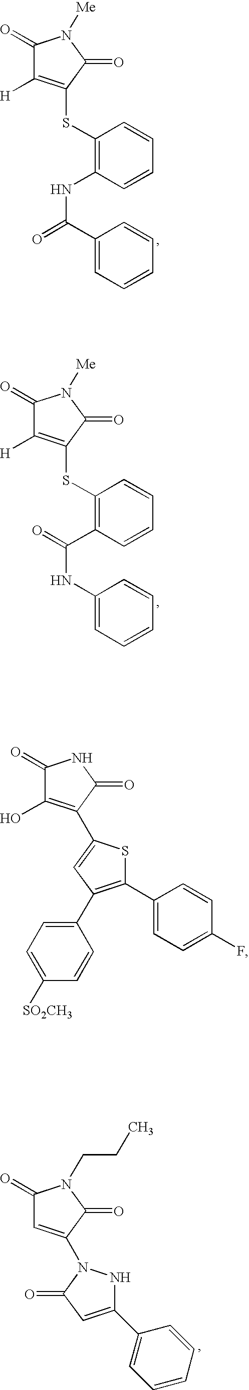 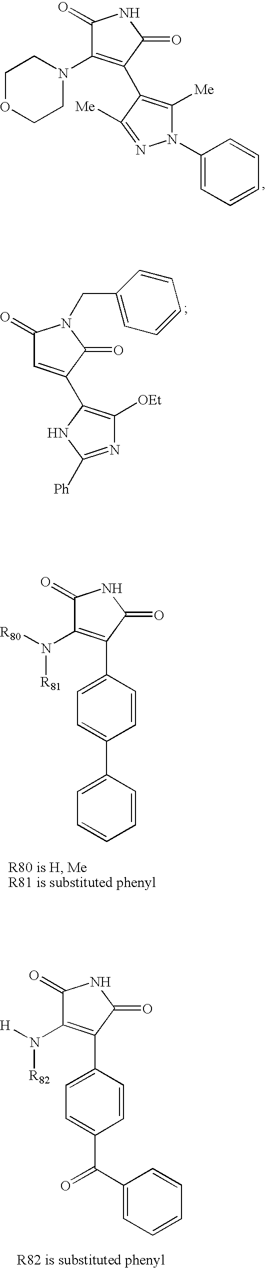
when Q is Q-8, then Y is not —CH₂O—;
when Q is Q-8, the compound of formula (I) is not 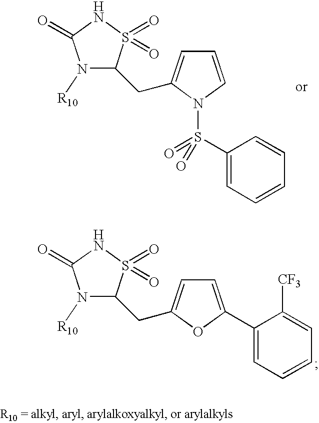
when Q is Q-9, then the compound of formula (I) is not 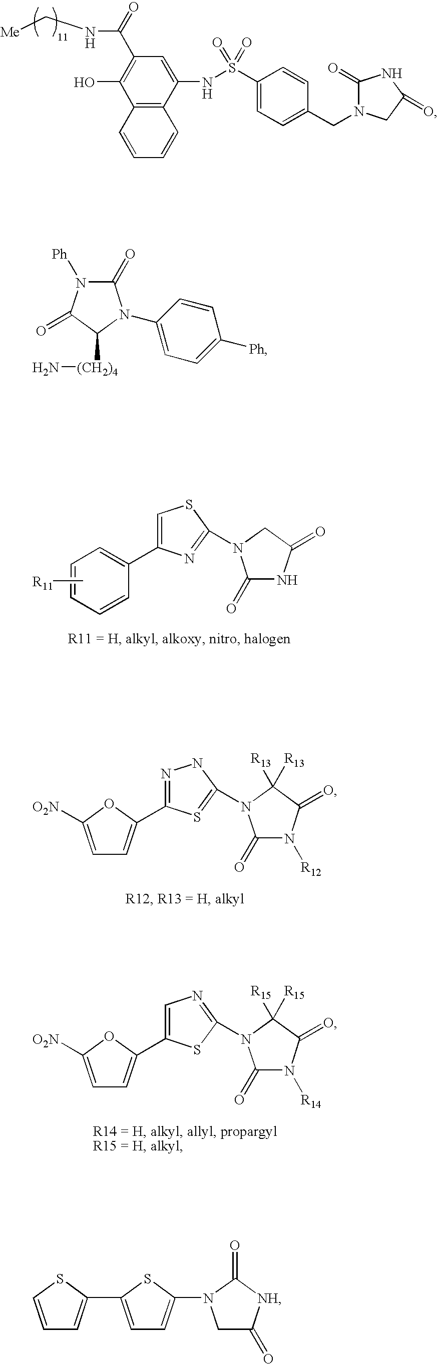 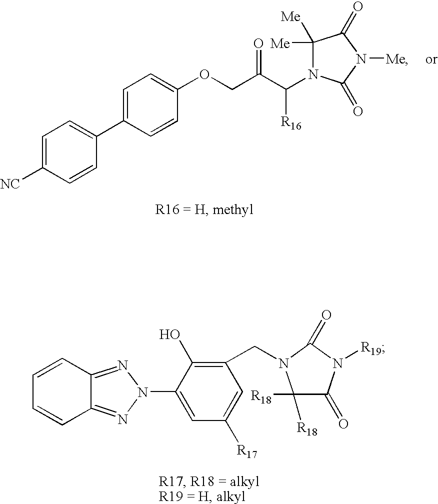
when Q is Q-10, t is 0, and B is phenyl, then any R₇ on E is not an o-alkoxy;
when Q is Q-10, then the compound of formula (I) is not 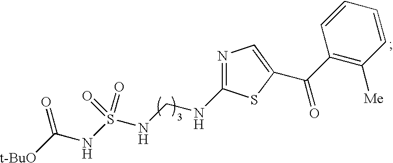
   when Q is Q-11, t is 0, and B is phenyl, then any R₇ on B is not an o-alkoxy;
   when Q is Q-11, then the compound of formula (I) is not 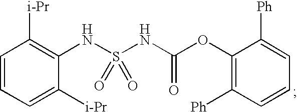
   when Q is Q-15, then the compound of formula (I) is not 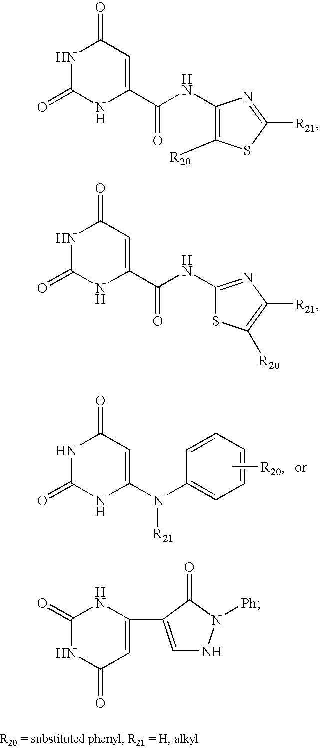
   when Q is Q-16 and Y is —NH—, then 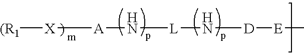
      of formula (I) is not biphenyl;
   when Q is Q-16 and Y is —S—, then 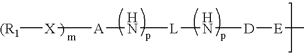
   of formula (I) is not phenylsulfonylaminophenyl or phenylcarbonylaminophenyl;
   when Q is Q-16 and Y is —SO₂NH—, then the compound of formula (I) is not 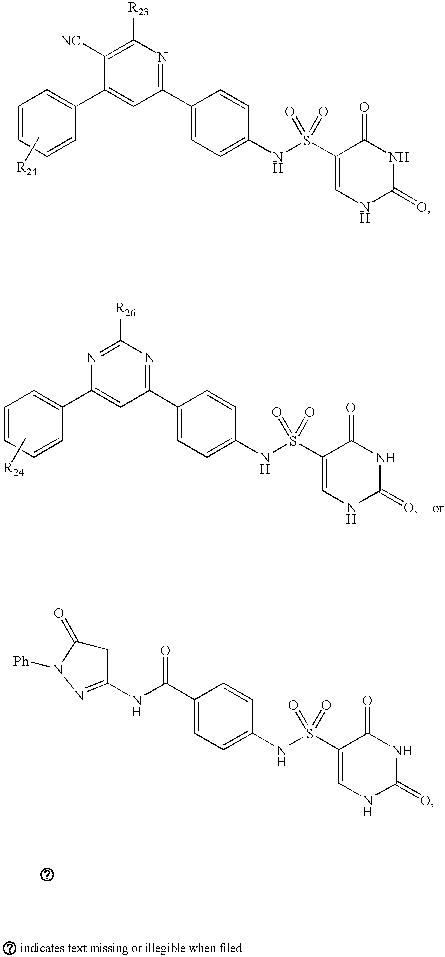
   when Q is Q-16 and Y is —CONH—, then 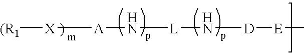
   of formula (I) is not imidazophenyl;
   when Q is Q-16 and Y is —CONH—, then the compound of formula (I) is not 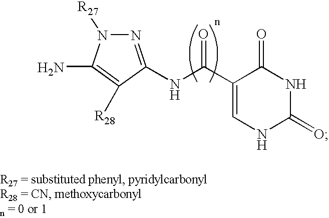
   when Q is Q-16 and t is 0, then 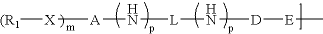
   of formula (I) is not phenylcarbonylphenyl, pyrimidophenyl, phenylpyrimidyl, 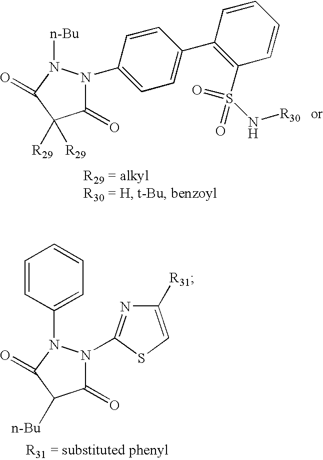
   when Q is Q-17, then the compound of formula (I) is not 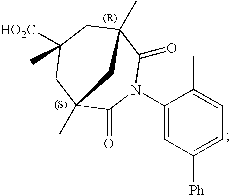
   when Q is Q-22, then the compound of formula (I) is selected from the group consisting of 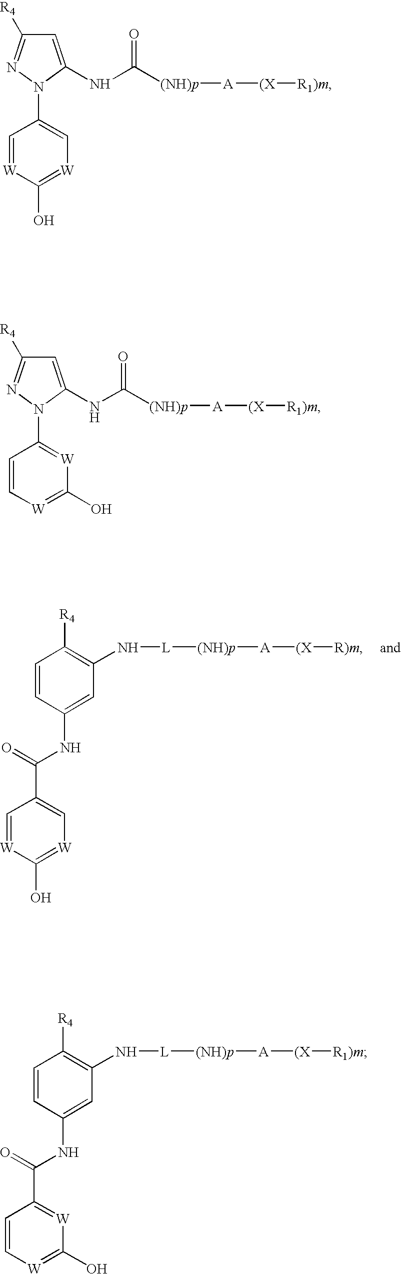
   when Q is Q-22 and q is 0, then the compound of formula (D) is selected from the group consisting of 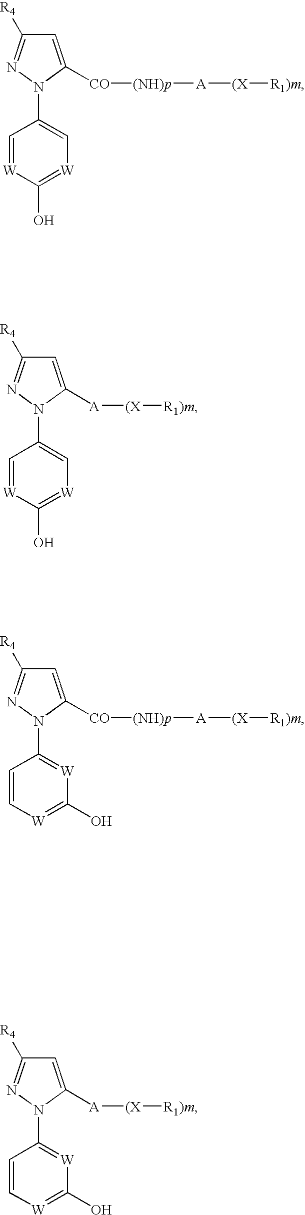 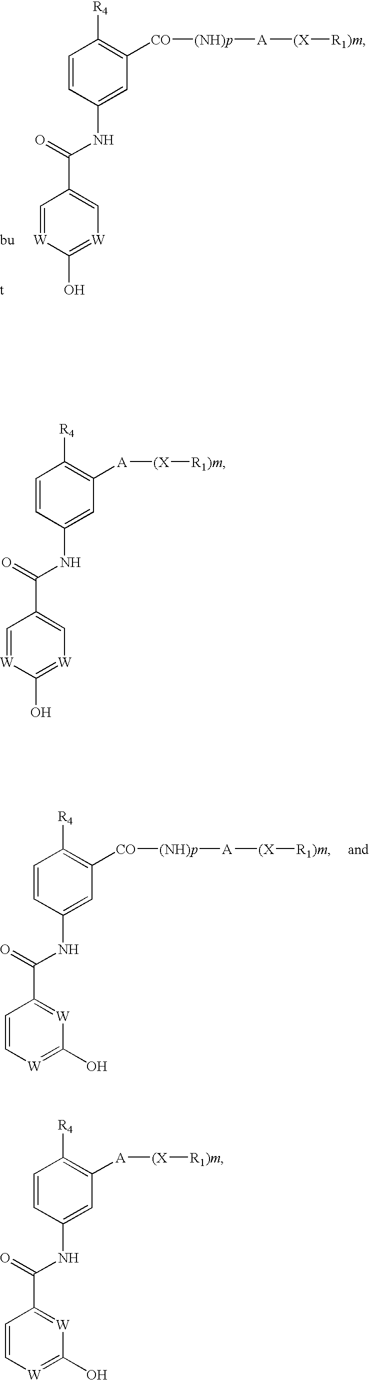
      excluding 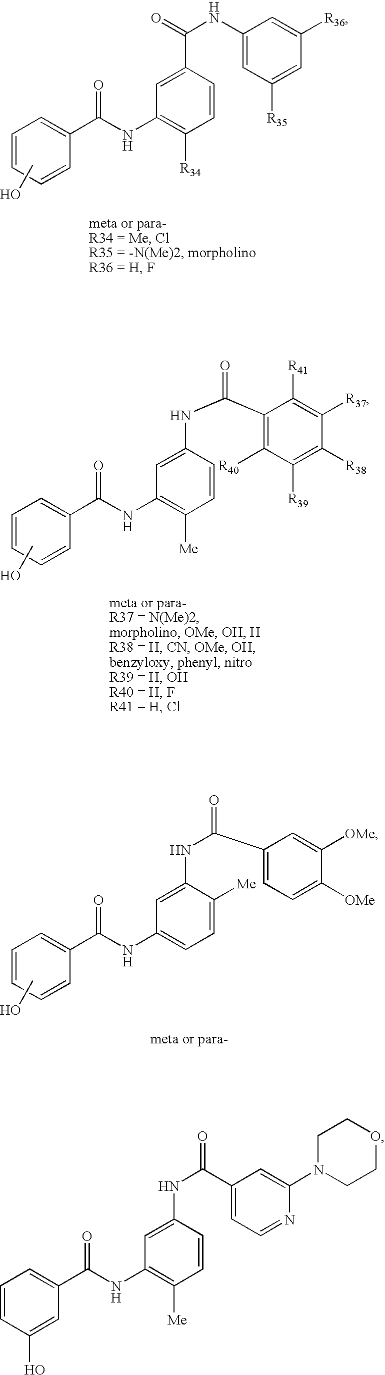 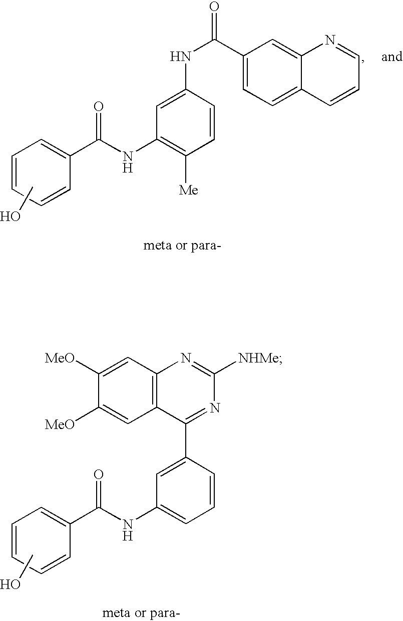
   when Q is Q-23, then the compound of formula (I) is not 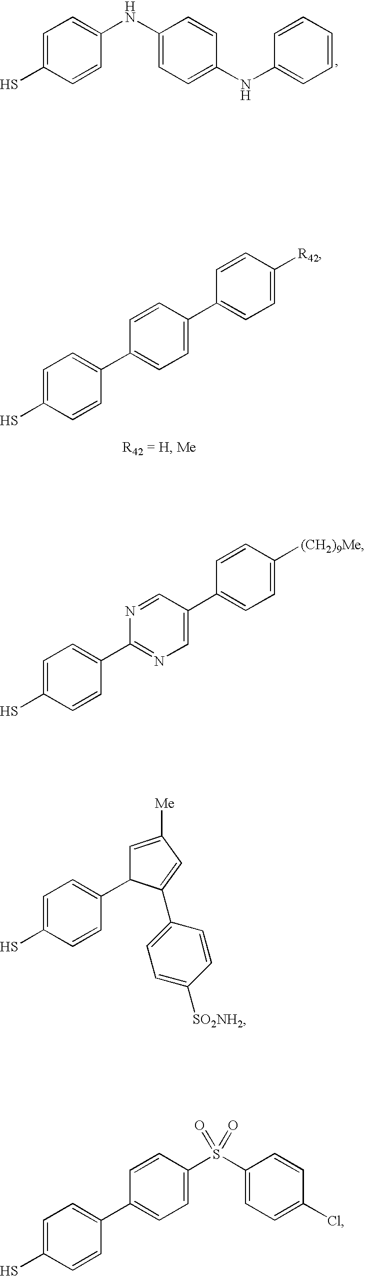 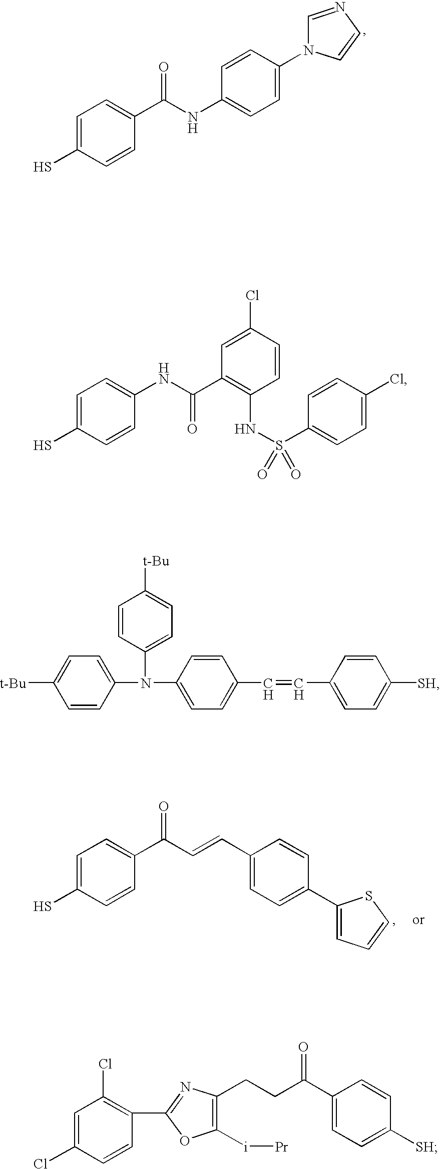
   when Q is Q-24, Q-25, Q-26, or Q-31, then the compound of formula (I) is selected from the group consisting of 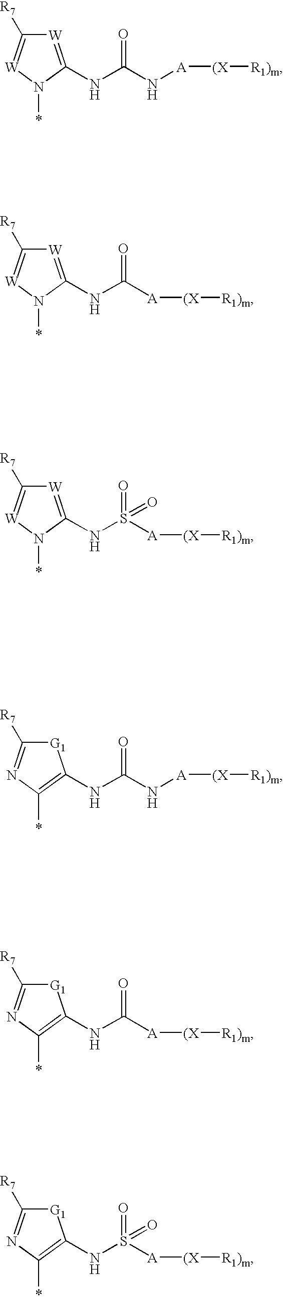 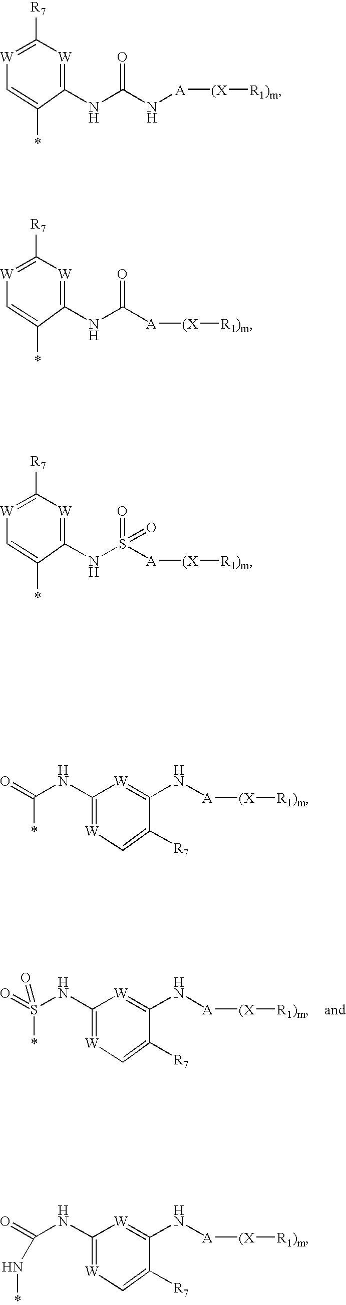
      wherein each W is individually selected from the group consisting of —CH— and —N—;
   each G₁ is individually selected from the group consisting of —O—, —S—, and —N(R₄)—; and
   * denotes the point of attachment to Q-24, Q-25, Q-26, or Q-31 as follows: 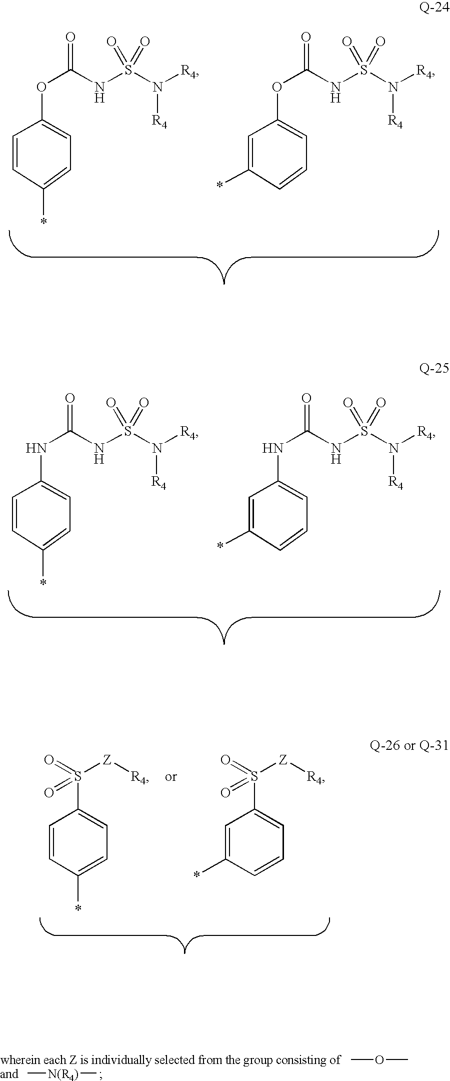
   when Q is Q-31, then the compound of formula (I) is not 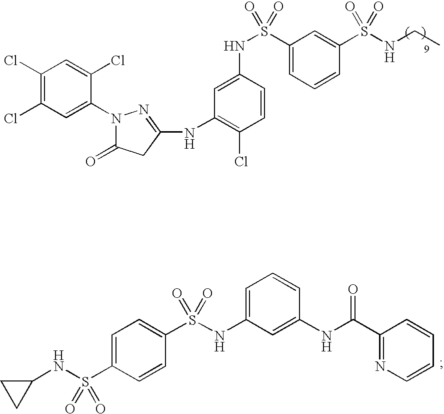
   when Q is Q-28 or Q-29 and t is 0, then the compound of formula (I) is not 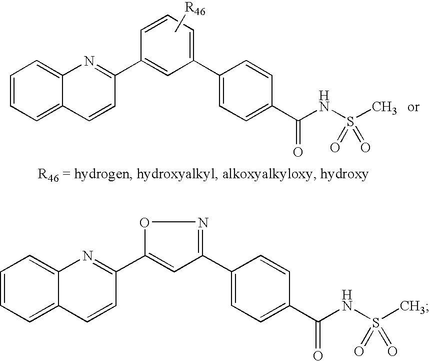
   when Q is Q-28 or Q-29 and Y is an ether linkage, then the compound of formula (I) is not 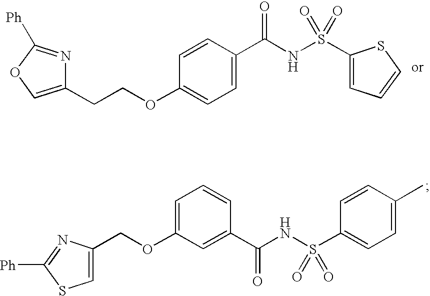
   when Q is Q-28 or Q-29 and Y is —CONH—, then the compound of formula (D) is not 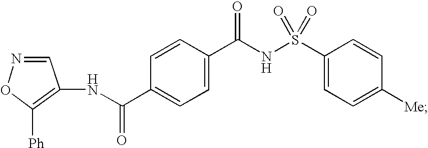
   when Q is Q-32, then 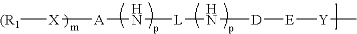
   is not biphenyl, benzoxazolylphenyl, pyridylphenyl or bipyridyl;
   when Q is Q-32, Y is —CONH—, q is 0, m is 0, and 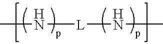
      of formula (I) is —CONH—, then A is not phenyl;
   when Q is Q-32, q is 0, m is 0, and 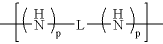
      is —CONH—, then the compound of formula (I) is not 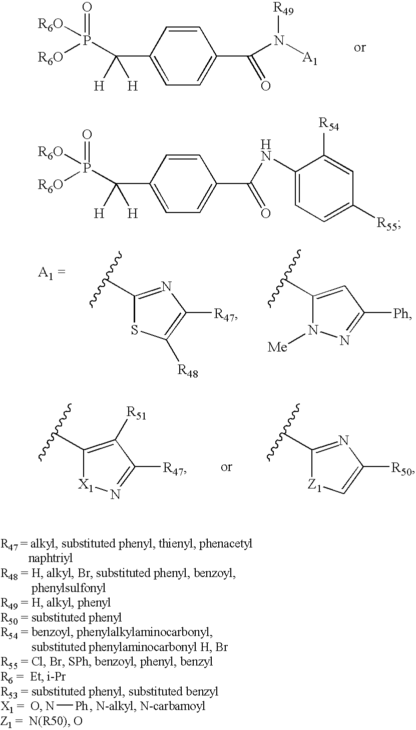
   when Q is Q-32, D is thiazolyl, q is 0, t is 0, p is 0, n is 0, and m is 0, then A is not phenyl or 2-pyridone;
   when Q is Q-32, D is oxazolyl or isoxazolyl, q is 0, t is 0, p is 0, n is 0, and m is 0, then A is not phenyl;
   when Q is Q-32, D is pyrimidyl q is 0, t is 0, p is 0, n is 0, and m is 0, then A is not phenyl;
   when Q is Q-32 and Y is an ether linkage, then 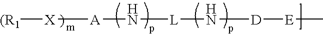
      of formula (I) is not biphenyl or phenyloxazolyl;
   when Q is Q-32 and Y is —CH═CH—, then 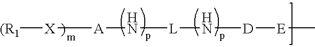
      of formula (I) is not phenylaminophenyl;
   when Q is Q-32, then the compound of formula (I) is not 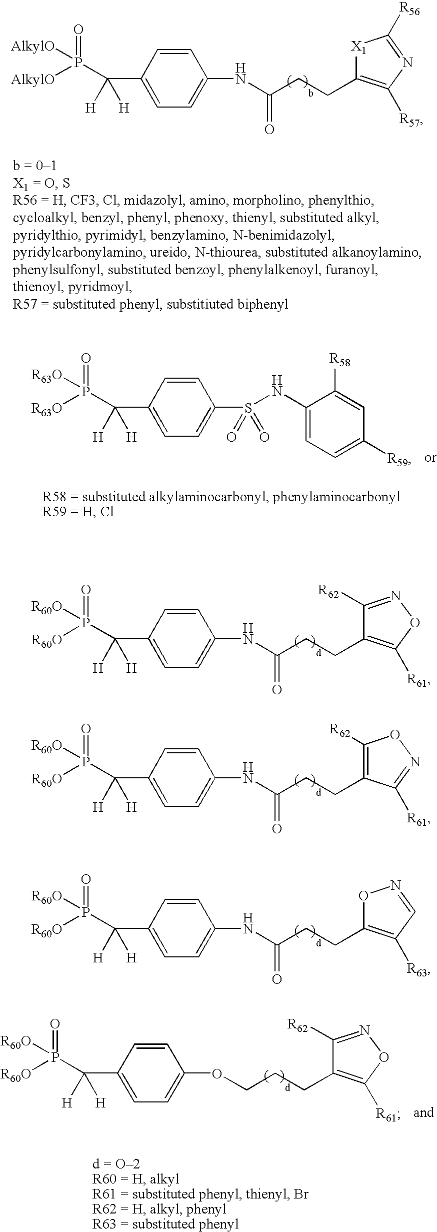
   when Q is Q-35 as shown 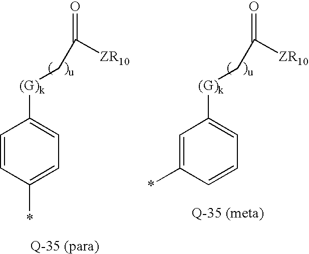
   wherein G is selected from the group consisting of —O—, —S—, —NR₄—, and —CH₂—, k is 0 or 1, and u is 1, 2, 3, or 4, then 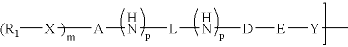
      is selected from the group consisting of 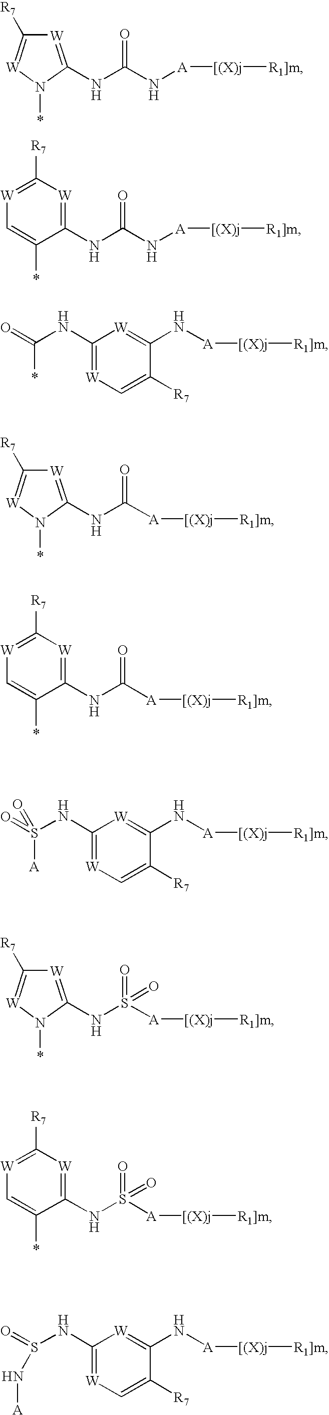 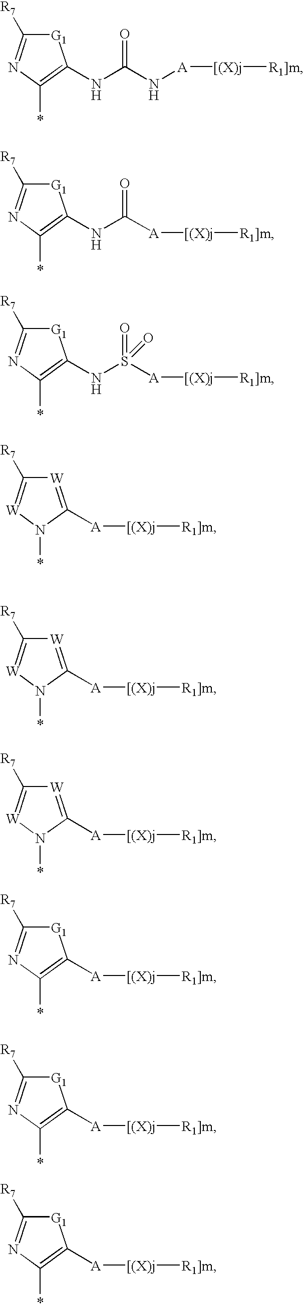 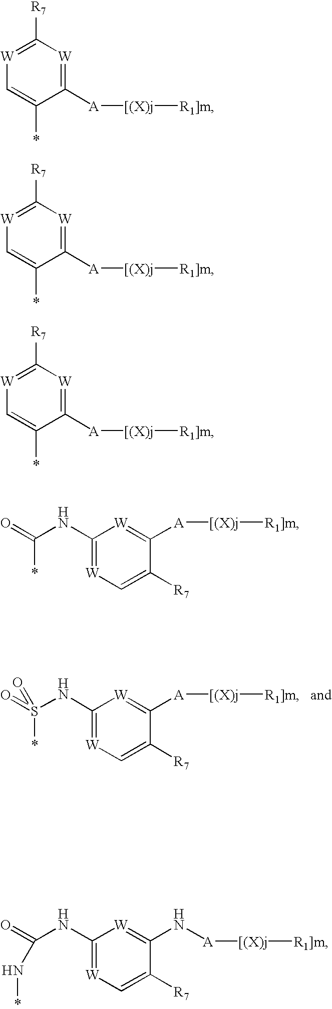
   except that the compound of formula (I) is not

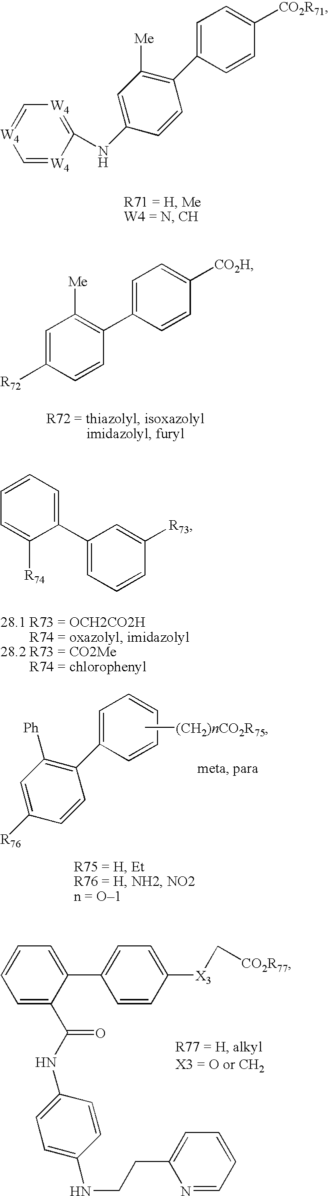

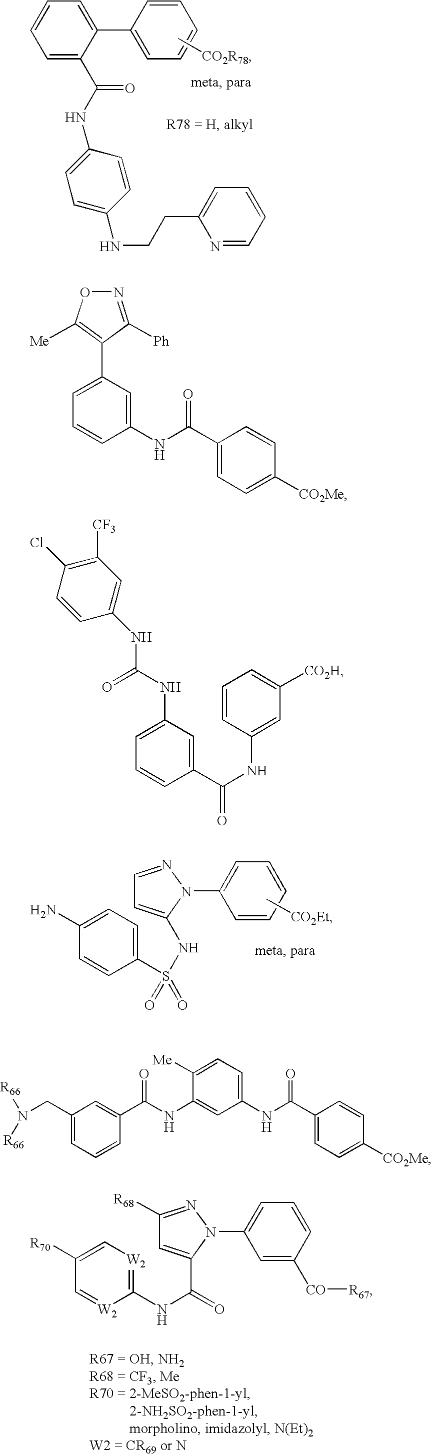

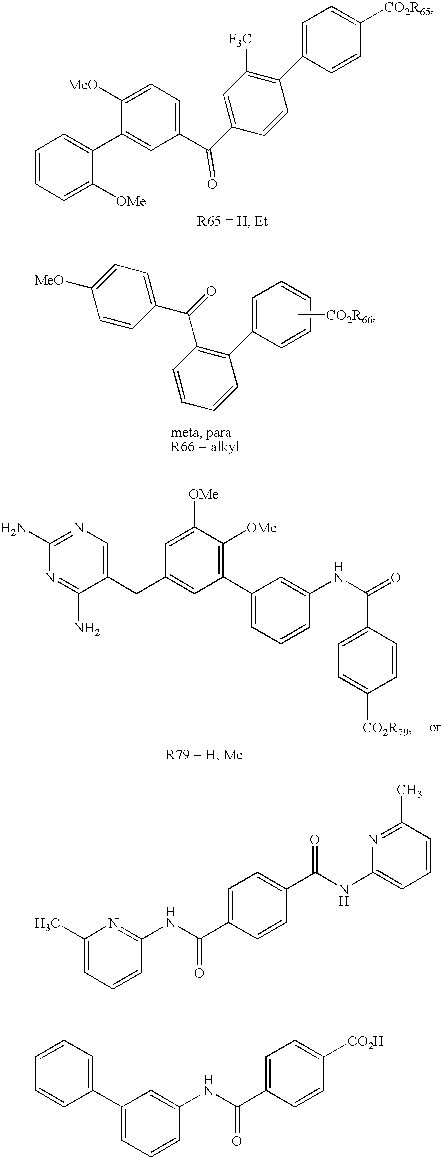

Even more preferably, R₁ as discussed above is selected from the group consisting of 6-5 fused heteroaryls, 6-5 fused heterocyclyls, 5-6 fused heteroaryls, and 5-6 fused heterocyclyls, and even more preferably, R₁ is selected from the group consisting of 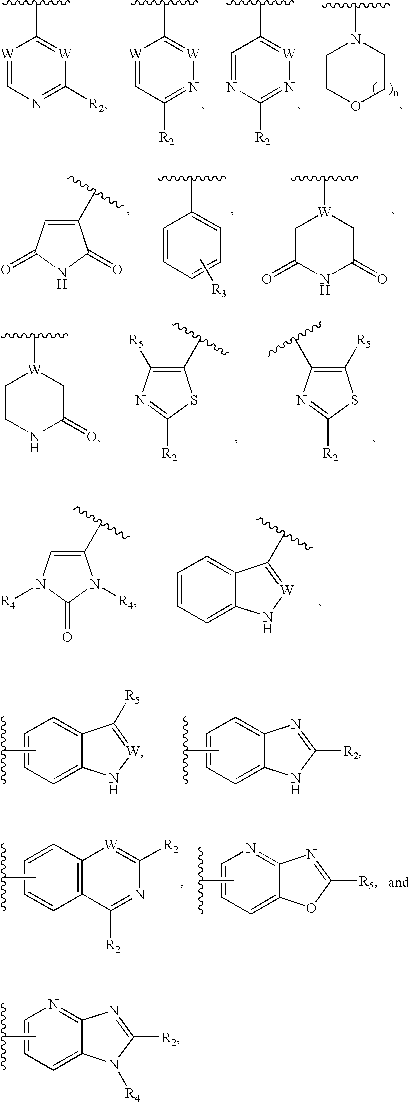
each R₂ is individually selected from the group consisting of —H, alkyls (preferably C₁-C₁₈, and more preferably C₁-C₁₂), aminos, alkylaminos (preferably C₁-C₁₈, and more preferably C₁-C₁₂), arylaminos (preferably C₆-C₁₈, and more preferably C₆-C₁₂), cycloalkylaminos (preferably C₁-C₁₈, and more preferably C₁-C₁₂), heterocyclylaminos, halogens, alkoxys (preferably C₁-C₁₈, and more preferably C₁-C₁₂), and hydroxys; and
each R₃ is individually selected from the group consisting of —H, alkyls (preferably C₁-C₁₈, and more preferably C₁-C₁₂), alkylaminos (preferably C₁-C₁₈, and more preferably C₁-C₁₂), arylaminos (preferably C₆-C₁₈, and more preferably C₆-C₁₂), cycloalkylaminos (preferably C₁-C₁₈, and more preferably C₁-C₁₂), heterocyclylaminos, alkoxys (preferably C₁-C₁₈, and more preferably C₁-C₁₂), hydroxys, cyanos, halogens, perfluoroalkyls (preferably C₁-C₁₈, and more preferably C₁-C₁₂), alkylsulfinyls (preferably C₁-C₁₈, and more preferably C₁-C₁₂), alkylsulfonyls (preferably C₁-C₁₈, and more preferably C₁-C₁₂), R₄NHSO₂—, and —NHSO₂R₄.

Finally, in another preferred embodiment, wherein A is selected from the group consisting of aromatic, monocycloheterocyclic, and bicycloheterocyclic rings; and most preferably phenyl, naphthyl, pyridyl, pyrimidyl, thienyl, furyl, pyrrolyl, thiazolyl, isothiazolyl, oxaxolyl, isoxazolyl, imidazolyl, oxadiazolyl, thiadiazolyl, indolyl, indazolyl, benzimidazolyl, benzotriazolyl, isoquinolyl, quinolyl, benzothiazolyl, benzofuranyl, benzothienyl, pyrazolylpyrimidinyl, imidazopyrimidinyl, purinyl, and

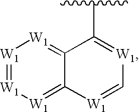

where each W₁ is individually selected from the group consisting of —CH— and —N—.

An additional class of compounds useful in the invention have the formula
A-T-(L)ₙ-(NH)ₚ-D-(E)_{q}-(Y)ₜ-Q (IB)
wherein:
Y is selected from the group consisting of —O—, —S—, —NR₆—, —NR₆SO₂—, —NR₆CO—, alkynyls (preferably C₁-C₁₈, and more preferably C₁-C₁₂), alkenyls (preferably C₁-C₁₈, and more preferably C₁-C₁₂), alkylenes (preferably C₁-C₁₈, and more preferably C₁-C₁₂), —O(CH₂)ₕ—, and —NR₆(CH₂)ₕ—, where each h is individually selected from the group consisting of 1, 2, 3, or 4, and where for each of alkylenes (preferably C₁-C₁₈, and more preferably C₁-C₁₂), —O(CH₂)ₕ—, and —NR₆(CH₂)ₕ—, one of the methylene groups present therein may be optionally double-bonded to a side-chain oxo group except that where —O(CH₂)ₕ— the introduction of the side-chain oxo group does not form an ester moiety;
A is selected from the group consisting of aromatic (preferably C₆-C₁₈, and more preferably C₆-C₁₂), monocycloheterocyclic, and bicycloheterocyclic rings; and most preferably phenyl, naphthyl, pyridyl, pyrimidyl, thienyl, furyl, pyrrolyl, thiazolyl, isothiazolyl, oxaxolyl, isoxazolyl, imidazolyl, oxadiazolyl, thiadiazolyl, indolyl, indazolyl, benzimidazolyl, benzotriazolyl, isoquinolyl, quinolyl, benzothiazolyl, benzofuranyl, benzothienyl, pyrazolylpyrimidinyl, imidazopyrimidinyl, purinyl, and 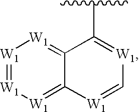
where each W1 is individually selected form the group consisting of —CH— and —N—.
D is phenyl or a five- or six-membered heterocyclic ring selected from the group consisting of pyrazolyl, pyrrolyl, imidazolyl, oxazolyl, thiazolyl, furyl, oxadiazolyl, thiadiazolyl, thienyl, pyridyl, and pyrimidyl;
E is selected from the group consisting of phenyl, pyridinyl, and pyrimidinyl;
L is selected from the group consisting of —C(O)— and —S(O)₂—;
T is NR₆, O, alkylene (preferably C₁-C₁₂, more preferably C₁-C₄), —O(CH₂)ₕ—, or —NR₆(CH₂)ₕ—, where each h is individually selected from the group consisting of 1, 2, 3, or 4, or T is absent wherein A is directly bonded to -(L)ₙ(NH)ₚ-D-(E)_{q}-(Y)ₜ-Q;
n is 0 or 1;
p is 0 or 1;
q is 0 or 1;
t is 0 or 1;
v is 1, 2, or 3;
x is 1 or 2;
Q is selected from the group consisting of formulae Q₃₆-Q₅₉, inclusive.
each R₄ group is individually selected from the group consisting of —H, alkyls (preferably C₁-C₁₈, and more preferably C₁-C₁₂), aminoalkyls (preferably C₁-C₁₈, and more preferably C₁-C₁₂), alkoxyalkyls (preferably C₁-C₁₈, and more preferably C₁-C₁₂), aryls (preferably C₆-C₁₈, and more preferably C₆-C₁₂), aralkyls (preferably C₆-C₁₈, and more preferably C₆-C₁₂ and preferably C₁-C₁₈, and more preferably C₁-C₁₂), heterocyclyls, and heterocyclylalkyls except when the R₄ substituent places a heteroatom on an alpha-carbon directly attached to a ring nitrogen on Q;
when two R₄ groups are bonded with the same atom, the two R₄ groups optionally form an alicyclic or heterocyclic 4-7 membered ring;
each R₆ is individually selected from the group consisting of —H, alkyls (preferably C₁-C₁₈, and more preferably C₁-C₁₂), allyls, and B-trimethylsilylethyl;
each R₈ is individually selected from the group consisting of alkyls (preferably C₁-C₁₈, and more preferably C₁-C₁₂), phenyl, naphthyl, aralkyls (wherein the aryl is preferably C₆-C₁₈, and more preferably C₆-C₁₂), wherein alkyl is preferably C₁-C₁₈, and more preferably C₁-C₁₂), heterocyclyls, and heterocyclylalkyls (wherein the alkyl is preferably C₁-C₁₈, and more preferably C₁-C₁₂);
each R₉ group is individually selected from the group consisting of —H, —F, and alkyls (preferably C₁-C₁₈, and more preferably C₁-C₁₂), wherein when two R₉ groups are geminal alkyl groups, said geminal alkyl groups may be cyclized to form a 3-6 membered ring;
each R_{9′} group is individually selected from the group consisting of —F, and alkyls (preferably C₁-C₁₈, and more preferably C₁-C₁₂), wherein when two R_{9′} groups are geminal alkyl groups, said geminal alkyl groups may be cyclized to form a 3-6 membered ring;
each R₁₀ is alkyl or perfluoroalkyl;
G is alkylene (preferably C₁-C₈, and more preferably C₁-C₄), N(R₆), O;
each Z is individually selected from the group consisting of —O— and —N(R₄)—;
and each ring of formula (I) optionally includes one or more of R_{7′}, where R_{7′} is a substituent individually selected from the group consisting of —H, alkyls (preferably C₁-C₁₈, and more preferably C₁-C₁₂), aryls (preferably C₆-C₁₈, and more preferably C₆-C₁₂), heterocyclyls, alkylaminos (preferably C₁-C₁₈, and more preferably C₁-C₁₂), arylaminos (preferably C₆-C₁₈, and more preferably C₆-C₁₂), cycloalkylaminos (preferably C₁-C₁₈, and more preferably C₁-C₁₂), heterocyclylaminos, hydroxys, alkoxys (preferably C₁-C₁₈, and more preferably C₁-C₁₂), perfluoroalkoxys (preferably C₁-C₈, more preferably C₁-C₄), aryloxys (preferably C₆-C₁₈, and more preferably C₆-C₁₂), alkylthios (preferably C₁-C₁₈, and more preferably C₁-C₁₂), arthylthios, cyanos, halogens, nitrilos, nitros, alkylsulfinyls (preferably C₁-C₁₈, and more preferably C₁-C₁₂), alkylsulfonyls (preferably C₁-C₁₈, and more preferably C₁-C₁₂), aminosulfonyls, perfluoroalkyls (preferably C₁-C₁₈, and more preferably C₁-C₁₂); aminooxaloylamino; alkylaminooxaloylamino; dialkylaminooxaloylamino; morpholinooxaloylamino; piperazinooxaloylamino; alkoxycarbonylamino; heterocyclyloxycarbonylamino; heterocyclylalkyloxycarbonylamino; heterocyclylcarbonylamino; heterocyclylalkylcarbonylamino; aminoalkyloxycarbonylamino; alkylaminoalkyloxycarbonylamino; or dialkylaminoalkyloxycarbonylamino;

In a preferred embodiment, A as described above is selected from the group consisting of phenyl, naphthyl, pyridyl, pyrimidyl, thienyl, furyl, pyrrolyl, pyrazolyl, thiazolyl, thiadiazolyl, oxazolyl, oxadiazolyl, imidazolyl, indolyl, indazolyl, benzimidazolyl, benzotriazolyl, isoquinolyl, quinolyl, benzothiazolyl, benzofuranyl, benzothienyl, pyrazolylpyrimidinyl, imidazopyrimidinyl, purinyl, and

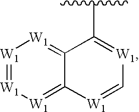

where each W₁ is individually selected from the group consisting of —CH— and —N—.

Q groups Q-36 through Q-59 are set forth below. In an additionally preferred embodiment, Q is taken from Q-37, Q-39, Q-41, Q-42, Q-43, Q-44, Q-47, Q-48, Q-54, and Q-57; in a more preferred embodiment, Q is taken from Q-39, Q-41, Q-42, Q-43, Q-44, Q-47, Q-48, and Q-54.

With respect to the method of using the novel compounds, the activation state of a kinase is determined by the interaction of switch control ligands and complemental switch control pockets. One conformation of the kinase may result from the switch control ligand's interaction with a particular switch control pocket while another conformation may result from the ligand's interaction with a different switch control pocket. Generally interaction of the ligand with one pocket, such as the “on” pocket, results in the kinase assuming an active conformation wherein the kinase is biologically active. Similarly, an inactive conformation (wherein the kinase is not biologically active) is assumed when the ligand interacts with another of the switch control pockets, such as the “off” pocket. The switch control pocket can be selected from the group consisting of simple, composite and combined switch control pockets. Interaction between the switch control ligand and the switch control pockets is dynamic and therefore, the ligand is not always interacting with a switch control pocket. In some instances, the ligand is not in a switch control pocket (such as occurs when the protein is changing from an active conformation to an inactive conformation). In other instances, such as when the ligand is interacting with the environment surrounding the protein in order to determine with which switch control pocket to interact, the ligand is not in a switch control pocket. Interaction of the ligand with particular switch control pockets is controlled in part by the charge status of the amino acid residues of the switch control ligand. When the ligand is in a neutral charge state, it interacts with one of the switch control pockets and when it is in a charged state, it interacts with the other of the switch control pockets. For example, the switch control ligand may have a plurality of OH groups and be in a neutral charge state. This neutral charge state results in a ligand that is more likely to interact with one of the switch control pockets through hydrogen boding between the OH groups and selected residues of the pocket, thereby resulting in whichever protein conformation results from that interaction. However, if the OH groups of the switch control ligand become charged through phosphorylation or some other means, the propensity of the ligand to interact with the other of the switch control pockets will increase and the ligand will interact with this other switch control pocket through complementary covalent binding between the negatively or positively charged residues of the pocket and ligand. This will result in the protein assuming the opposite conformation assumed when the ligand was in a neutral charge state and interacting with the other switch control pocket.

Of course, the conformation of the protein determines the activation state of the protein and can therefore play a role in protein-related diseases, processes, and conditions. For example, if a metabolic process requires a biologically active protein but the protein's switch control ligand remains in the switch control pocket (i.e. the “off” pocket) that results in a biologically inactive protein, that metabolic process cannot occur at a normal rate. Similarly, if a disease is exacerbated by a biologically active protein and the protein's switch control ligand remains in the switch control pocket (i.e. the “on” pocket) that results in the biologically active protein conformation, the disease condition will be worsened. Accordingly, as demonstrated by the present invention, selective modulation of the switch control pocket and switch control ligand by the selective administration of a molecule will play an important role in the treatment and control of protein-related diseases, processes, and conditions.

One aspect of the invention provides a method of modulating the activation state of a kinase, preferably p38 α-kinase and including both the consensus wild type sequence and disease polymorphs thereof. The activation state is generally selected from an upregulated or downregulated state. The method generally comprises the step of contacting the kinase with a molecule having the general formula (I). When such contact occurs, the molecule will bind to a particular switch control pocket and the switch control ligand will have a greater propensity to interact with the other of the switch control pockets (i.e., the unoccupied one) and a lesser propensity to interact with the occupied switch control pocket. As a result, the protein will have a greater propensity to assume either an active or inactive conformation (and consequently be upregulated or downregulated), depending upon which of the switch control pockets is occupied by the molecule. Thus, contacting the kinase with a molecule modulates that protein's activation state. The molecule can act as an antagonist or an agonist of either switch control pocket. The contact between the molecule and the kinase preferably occurs at a region of a switch control pocket of the kinase and more preferably in an interlobe oxyanion pocket of the kinase. In some instances, the contact between the molecule and the pocket also results in the alteration of the conformation of other adjacent sites and pockets, such as an ATP active site. Such an alteration can also effect regulation and modulation of the active state of the protein. Preferably, the region of the switch control pocket of the kinase comprises an amino acid residue sequence operable for binding to the Formula I molecule. Such binding can occur between the molecule and a specific region of the switch control pocket with preferred regions including the α-C helix, the α-D helix, the catalytic loop, the activation loop, and the C-terminal residues or C-lobe residues (all residues located downstream (toward the C-end) from the Activation loop), the glycine rich loop, and combinations thereof. When the binding region is the α-C helix, one preferred binding sequence in this helix is the sequence IIHXKRXXREXXLLXXM, (SEQ ID NO. 2). When the binding region is the catalytic loop, one preferred binding sequence in this loop is DIIHRD (SEQ ID NO. 3). When the binding region is the activation loop, one preferred binding sequence in this loop is a sequence selected from the group consisting of DFGLARHTDD (SEQ ID NO. 4), EMTGYVATRWYR (SEQ ID NO. 5), and combinations thereof. When the binding region is in the C-lobe residues, one preferred binding sequence is WMHY (SEQ ID NO. 6). When the binding region is in the glycine rich loop one preferred binding sequence is YGSV (SEQ ID NO. 7). When a biologically inactive protein conformation is desired, molecules which interact with the switch control pocket that normally results in a biologically active protein conformation (when interacting with the switch control ligand) will be selected. Similarly, when a biologically active protein conformation is desired, molecules which interact with the switch control pocket that normally results in a biologically inactive protein conformation (when interacting with the switch control ligand) will be selected. Thus, the propensity of the protein to assume a desired conformation will be modulated by administration of the molecule. In preferred forms, the molecule will be administered to an individual undergoing treatment for a condition selected from the group consisting of human inflammation, rheumatoid arthritis, rheumatoid spondylitis, ostero-arthritis, asthma, gouty arthritis, sepsis, septic shock, endotoxic shock, Gram-negative sepsis, toxic shock syndrome, adult respiratory distress syndrome, stroke, reperfusion injury, neural trauma, neural ischemia, psoriasis, restenosis, chronic pulmonary inflammatory disease, bone resorptive diseases, graft-versus-host reaction, Chron's disease, ulcerative colitis, inflammatory bowel disease, pyresis, and combinations thereof. In such forms, it will be desired to select molecules that interact with the switch control pocket that generally leads to a biologically active protein conformation so that the protein will have the propensity to assume the biologically inactive form and thereby alleviate the condition. It is contemplated that the molecules of the present invention will be administrable in any conventional form including oral, parenteral, inhalation, and subcutaneous. It is preferred for the administration to be in the oral form. Preferred molecules include the preferred compounds of formula (I), as discussed above.

Another aspect of the present invention provides a method of treating an inflammatory condition of an individual comprising the step of administering a molecule having the general formula (I) to the individual. Such conditions are often the result of an overproduction of the biologically active form of a protein, including kinases. The administering step generally includes the step of causing said molecule to contact a kinase involved with the inflammatory process, preferably p38 α-kinase. When the contact is between the molecule and a kinase, the contact preferably occurs in an interlobe oxyanion pocket of the kinase that includes an amino acid residue sequence operable for binding to the Formula I molecule. Preferred binding regions of the interlobe oxyanion pocket include the α-C helix region, the α-D helix region, the catalytic loop, the activation loop, the C-terminal residues, the glycine rich loop residues, and combinations thereof. When the binding region is the α-C helix, one preferred binding sequence in this helix is the sequence IIHXKRXXREXXLLXXM, (SEQ ID NO. 2). When the binding region is the catalytic loop, one preferred binding sequence in this loop is DIIHRD (SEQ ID NO. 3). When the binding region is the activation loop, one preferred binding sequence in this loop is a sequence selected from the group consisting of DFGLARHTDD (SEQ ID NO. 4), EMTGYVATRWYR (SEQ ID NO. 5), and combinations thereof. Such a method permits treatment of the condition by virtue of the modulation of the activation state of a kinase by contacting the kinase with a molecule that associates with the switch control pocket that normally leads to a biologically active form of the kinase when interacting with the switch control ligand. Because the ligand cannot easily interact with the switch control pocket associated with or occupied by the molecule, the ligand tends to interact with the switch control pocket leading to the biologically inactive form of the protein, with the attendant result of a decrease in the amount of biologically active protein. Preferably, the inflammatory condition is selected from the group consisting of human inflammation, rheumatoid arthritis, rheumatoid spondylitis, ostero-arthritis, asthma, gouty arthritis, sepsis, septic shock, endotoxic shock, Gram-negative sepsis, toxic shock syndrome, adult respiratory distress syndrome, stroke, reperfusion injury, neural trauma, neural ischemia, psoriasis, restenosis, chronic pulmonary inflammatory disease, bone resorptive diseases, graft-versus-host reaction, Chron's disease, ulcerative colitis, inflammatory bowel disease, pyresis, and combinations thereof. As with the other methods of the invention, the molecules may be administered in any conventional form, with any convention excipients or ingredients. However, it is preferred to administer the molecule in an oral dosage form. Preferred molecules are again selected from the group consisting of the preferred formula (I) compounds discussed above.

### BRIEF DESCRIPTION OF THE DRAWINGS

FIG. 1 is a schematic representation of a naturally occurring mammalian protein in accordance with the invention including “on” and “off” switch control pockets 102 and 104, respectively, a transiently modifiable switch control ligand 106, and an active ATP site 108;

FIG. 2 is a schematic representation of the protein of FIG. 1, wherein the switch control ligand 106 is illustrated in a binding relationship with the off switch control pocket 104, thereby causing the protein to assume a first biologically downregulated conformation;

FIG. 3 is a view similar to that of FIG. 1, but illustrating the switch control ligand 106 in its charged-modified condition wherein the OH groups 110 of certain amino acid residues have been phosphorylated;

FIG. 4 is a view similar to that of FIG. 2, but depicting the protein wherein the phosphorylated switch control ligand 106 is in a binding relationship with the on switch control pocket 102, thereby causing the protein to assume a second biologically-active conformation different than the first conformation of FIG. 2;

FIG. 4a is an enlarged schematic view illustrating a representative binding between the phosphorylated residues of the switch control ligand 106, and complemental residues Z+ from the on switch control pocket 102;

FIG. 5 is a view similar to that of FIG. 1, but illustrating in schematic form possible small molecule compounds 116 and 118 in a binding relationship with the off and on switch control pockets 104 and 102, respectively;

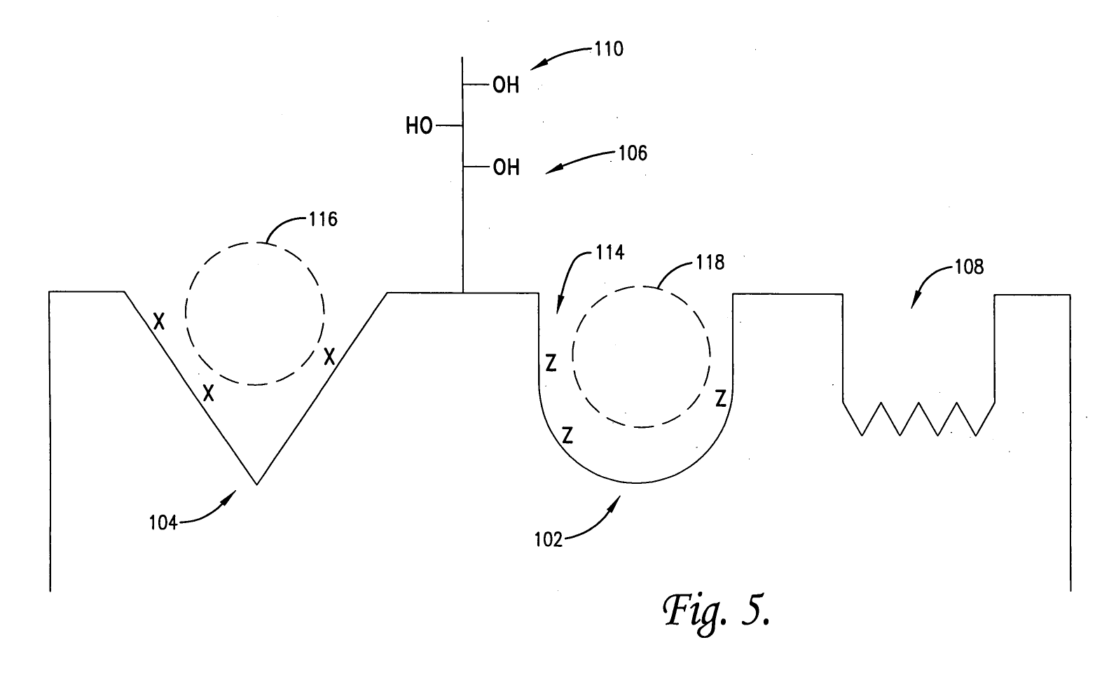

FIG. 6 is a schematic view of the protein in a situation where a composite switch control pocket 120 is formed with portions of the switch control ligand 106 and the on switch control pocket 102, and with a small molecule 122 in binding relationship with the composite pocket; and

FIG. 7 is a schematic view of the protein in a situation where a combined switch control pocket 124 is formed with portions of the on switch control pocket 102, the switch control ligand sequence 106, and the active ATP site 108, and with a small molecule 126 in binding relationship with the combined switch control pocket.

### DETAILED DESCRIPTION OF THE PREFERRED EMBODIMENTS

The present invention provides a way of rationally developing new small molecule modulators which interact with naturally occurring proteins (e.g., mammalian, and especially human proteins) in order to modulate the activity of the proteins. Novel protein-small molecule adducts are also provided. The invention preferably makes use of naturally occurring proteins having a conformational property whereby the proteins change their conformations in vivo with a corresponding change in protein activity. For example, a given enzyme protein in one conformation may be biologically upregulated, while in another conformation, the same protein may be biologically downregulated. The invention preferably makes use of one mechanism of conformation change utilized by naturally occurring proteins, through the interaction of what are termed “switch control ligands” and “switch control pockets” within the protein.

As used herein, “switch control ligand” means a region or domain within a naturally occurring protein and having one or more amino acid residues therein which are transiently modified in vivo between individual states by biochemical modification, typically phosphorylation, sulfation, acylation or oxidation. Similarly, “switch control pocket” means a plurality of contiguous or non-contiguous amino acid residues within a naturally occurring protein and comprising residues capable of binding in vivo with transiently modified residues of a switch control ligand in one of the individual states thereof in order to induce or restrict the conformation of the protein and thereby modulate the biological activity of the protein, and/or which is capable of binding with a non-naturally occurring switch control modulator molecule to induce or restrict a protein conformation and thereby modulate the biological activity of the protein.

A protein-modulator adduct in accordance with the invention comprises a naturally occurring protein having a switch control pocket with a non-naturally occurring molecule bound to the protein at the region of said switch control pocket, said molecule serving to at least partially regulate the biological activity of said protein by inducing or restricting the conformation of the protein. Preferably, the protein also has a corresponding switch control ligand, the ligand interacting in vivo with the pocket to regulate the conformation and biological activity of the protein such that the protein will assume a first conformation and a first biological activity upon the ligand-pocket interaction, and will assume a second, different conformation and biological activity in the absence of the ligand-pocket interaction.

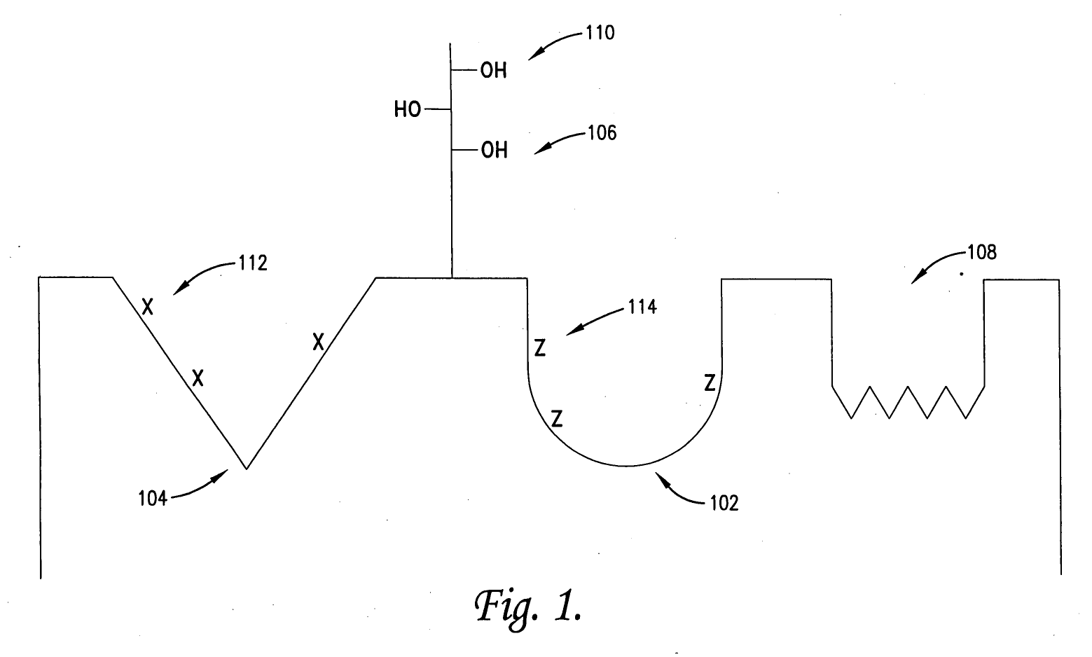

The nature of the switch control ligand/switch control pocket interaction may be understood from a consideration of schematic FIGS. 1-4. Specifically, in FIG. 1, a protein 100 is illustrated in schematic form to include an “on” switch control pocket 102, and “off” switch control pocket 104, and a switch control ligand 106. In addition, the schematically depicted protein also includes an ATP active site 108. In the exemplary protein of FIG. 1, the ligand 106 has three amino acid residues with side chain OH groups 110. The off pocket 104 contains corresponding X residues 112 and the on pocket 102 has Z residues 114. In the exemplary instance, the protein 100 will change its conformation depending upon the charge status of the OH groups 110 on ligand 106, i.e., when the OH groups are unmodified, a neutral charge is presented, but when these groups are phosphorylated a negative charge is presented.

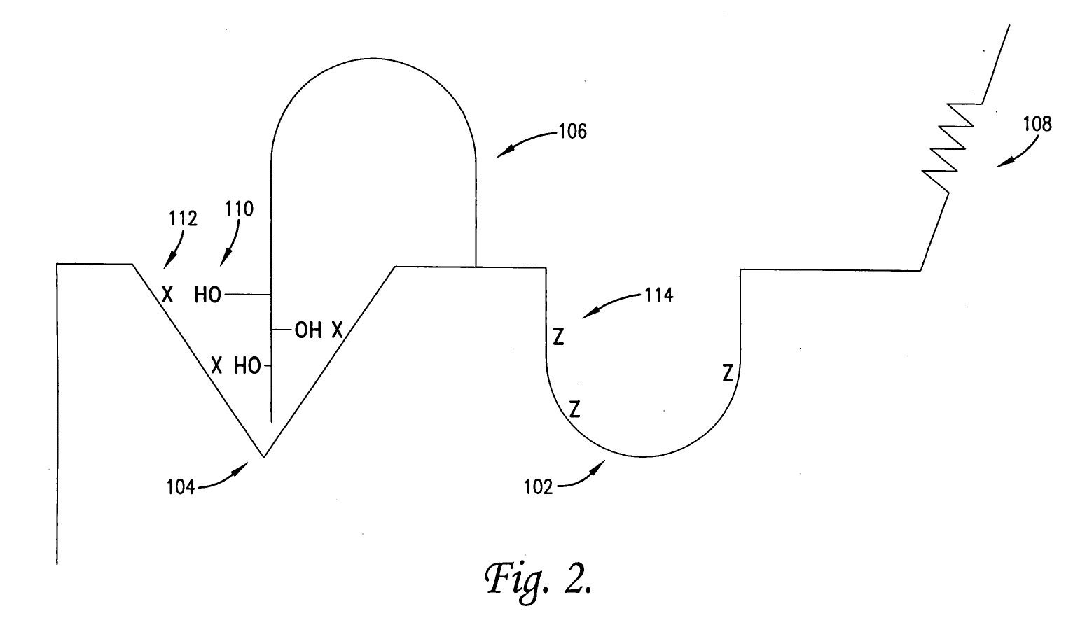

The functionality of the pockets 102, 104 and ligand 106 can be understood from a consideration of FIGS. 2-4. In FIG. 2, the ligand 106 is shown operatively interacted with the off pocket 104 such that the OH groups 110 interact with the X residues 112 forming a part of the pocket 104. Such interaction is primarily by virtue of hydrogen bonding between the OH groups 110 and the residues 112. As seen, this ligand/pocket interaction causes the protein 100 to assume a conformation different from that seen in FIG. 1 and corresponding to the off or biologically downregulated conformation of the protein.

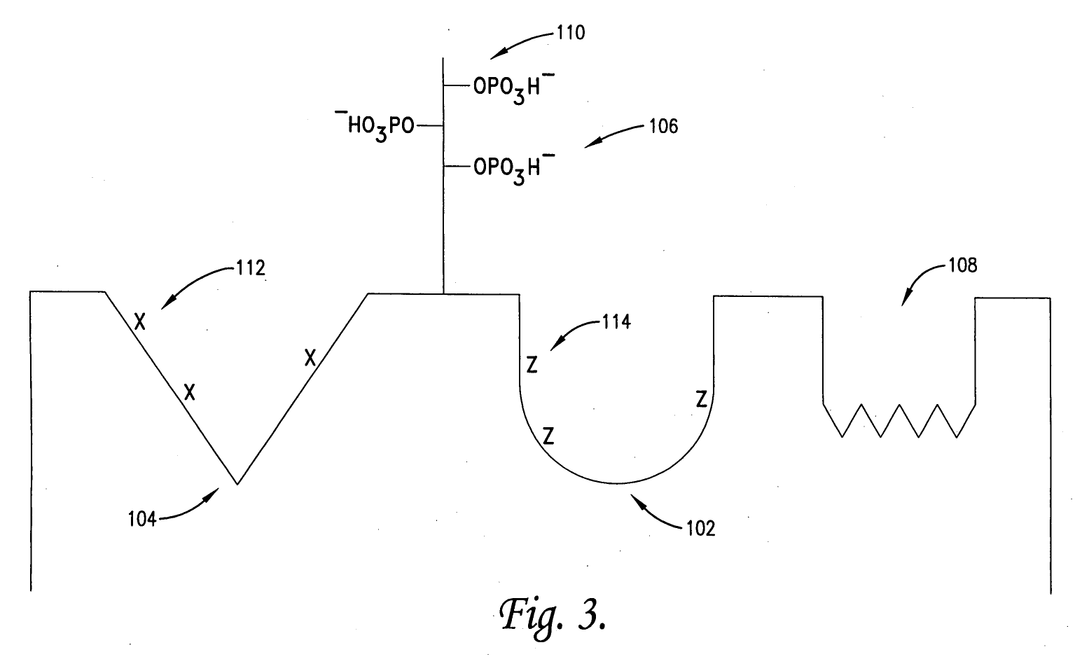

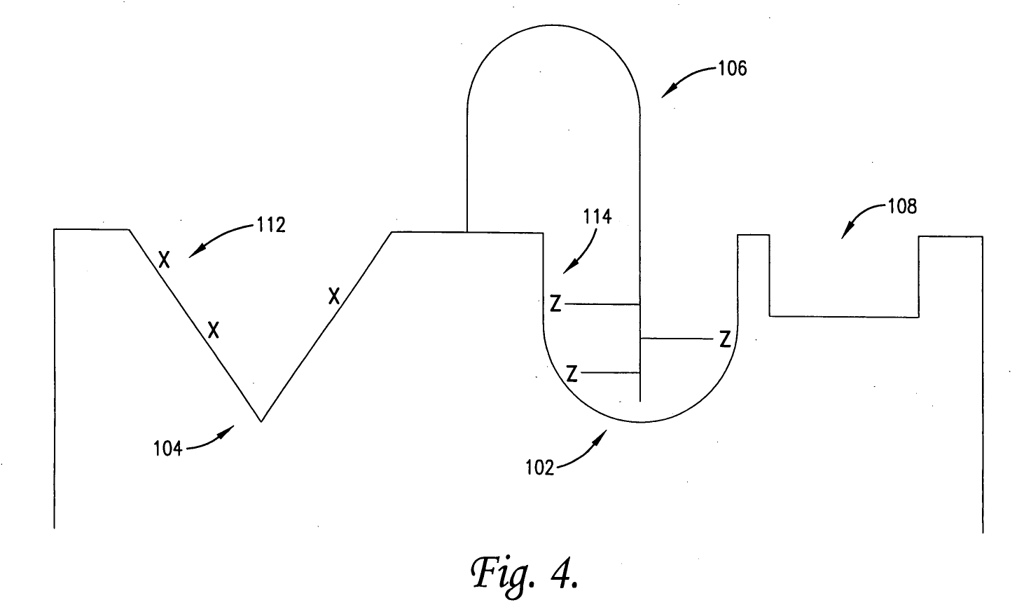

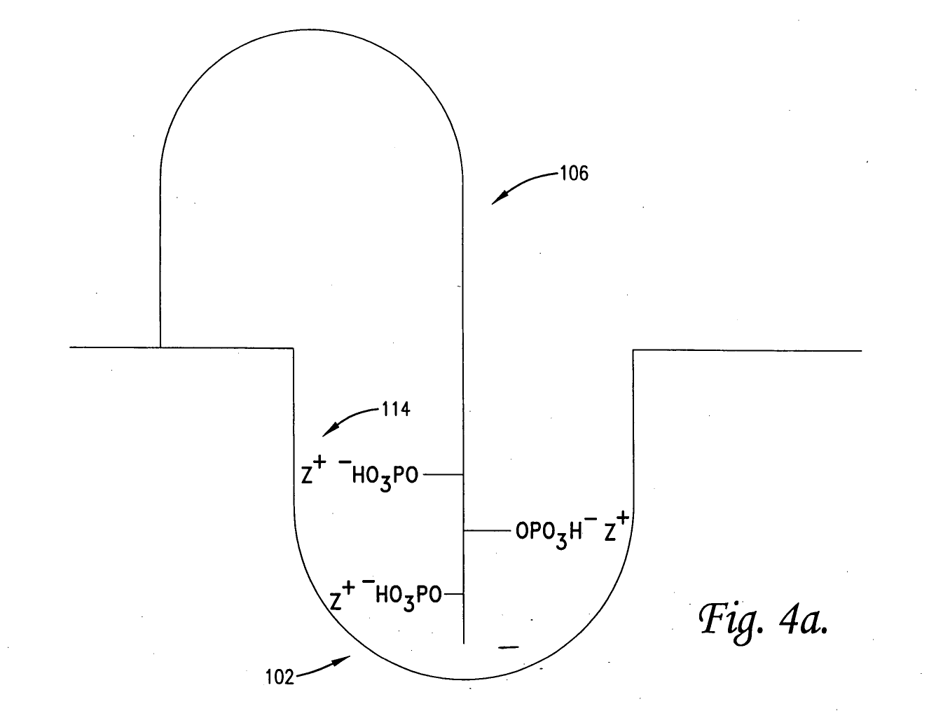

FIG. 3 illustrates the situation where the ligand 106 has shifted from the off pocket interaction conformation of FIG. 2 and the OH groups 110 have been phosphorylated, giving a negative charge to the ligand. In this condition, the ligand has a strong propensity to interact with on pocket 102, to thereby change the protein conformation to the on or biologically upregulated state (FIG. 4). FIG. 4a illustrates that the phosphorylated groups on the ligand 106 are attracted to positively charged residues 114 to achieve an ionic-like stabilizing bond. Note that in the on conformation of FIG. 4, the protein conformation is different than the off conformation of FIG. 2, and that the ATP active site is available and the protein is functional as a kinase enzyme.

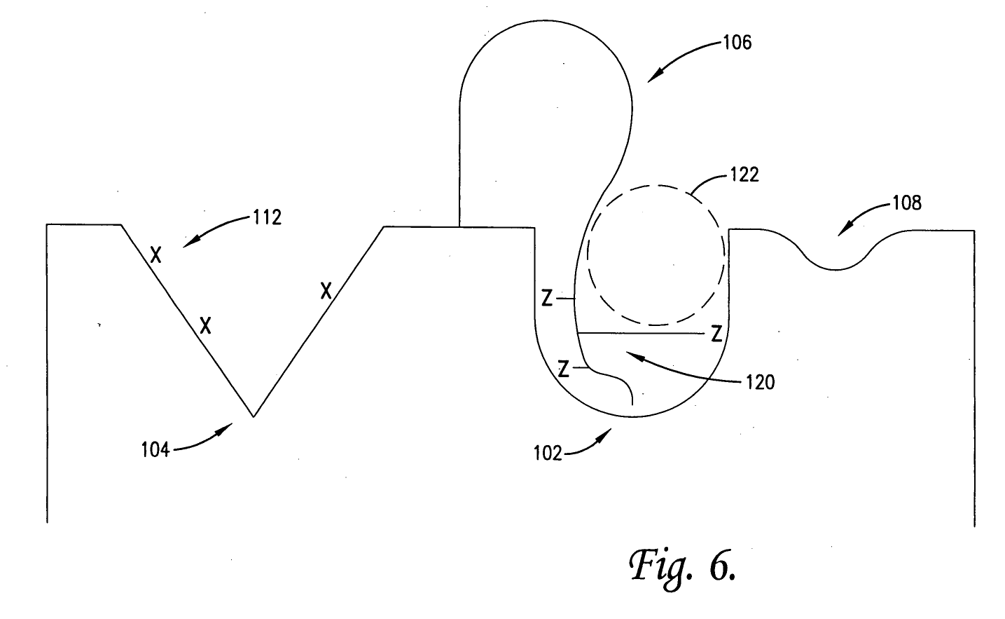

FIGS. 1-4 illustrate a simple situation where the protein exhibits discrete pockets 102 and 104 and ligand 106. However, in many cases a more complex switch control pocket pattern is observed. FIG. 6 illustrates a situation where an appropriate pocket for small molecule interaction is formed from amino acid residues taken both from ligand 106 and, for example, from pocket 102. This is termed a “composite switch control pocket” made up of residues from both the ligand 106 and a pocket, and is referred to by the numeral 120. A small molecule 122 is illustrated which interacts with the pocket 120 for protein modulation purposes.

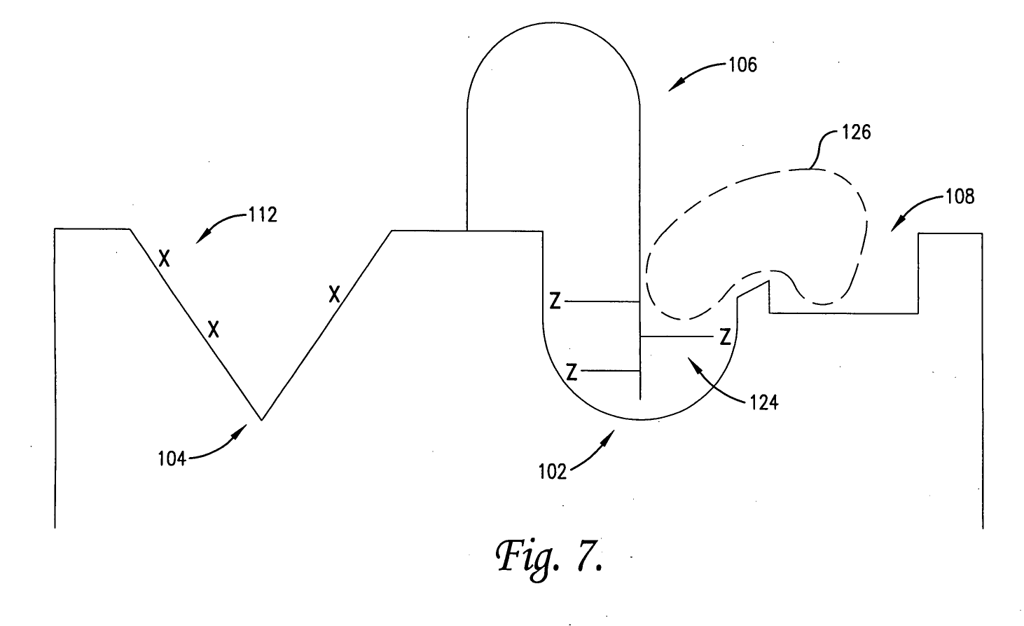

Another more complex switch pocket is depicted in FIG. 7 wherein the pocket includes residues from on pocket 102, and ATP site 108 to create what is termed a “combined switch control pocket.” Such a combined pocket is referred to as numeral 124 and may also include residues from ligand 106. An appropriate small molecule 126 is illustrated with pocket 124 for protein modulation purposes.

It will thus be appreciated that while in the simple pocket situation of FIGS. 1-4, the small molecule will interact with the simple pocket 102 or 104, in the more complex situations of FIGS. 6 and 7 the interactive pockets are in the regions of the pockets 120 or 124. Thus, broadly the small molecules interact “at the region” of the respective switch control pocket.

### Materials and Methods

### General Synthesis of Compounds

In the synthetic schemes of this section, q is 0 or 1. When q=0, the substituent is replaced by a synthetically non-interfering group R₇.

Compounds of Formula I wherein Q is taken from Q-1 or Q-2 and Y is alkylene are prepared according to the synthetic route shown in Scheme 1.1. Reaction of isothiocyanate 1 with chlorine, followed by addition of isocyanate 2 affords 3-oxo-thiadiazolium salt 3. Quenching of the reaction with air affords compounds of Formula I-4. Alternatively, reaction of isothiocyanate 1 with isothiocyanate 5 under the reaction conditions gives rise to compounds of Formula I-7. See A. Martinez et al, Journal of Medicinal Chemistry (2002) 45:1292.

Intermediates 1, 2 and 5 are commercially available or prepared according to Scheme 1.2. Reaction of amine 8 with phosgene or a phosgene equivalent affords isocyanate 2. Similarly, reaction of amine 8 with thiophosgene affords isothiocyanate 5. Amine 8 is prepared by palladium(0)-catalyzed amination of 9, wherein M is a group capable of oxidative insertion into palladium(0), according to methodology reported by S. Buchwald. See M. Wolter et al, Organic Letters (2002) 4:973; B. H. Yang and S. Buchwald, Journal of Organometallic Chemistry (1999) 576(1-2):125. In this reaction sequence, P is a suitable amine protecting group. Use of and removal of amine protecting groups is accomplished by methodology reported in the literature (Protective Groups in Organic Synthesis, Peter G. M. Wutts, Theodora Greene (Editors) 3rd edition (April 1999) Wiley, John & Sons, Incorporated; ISBN: 0471160199). Starting compounds 9 are commercially available or readily prepared by one of ordinary skill in the art: See March's Advanced Organic Chemistry: Reactions, Mechanisms, and Structure, Michael B. Smith & Jerry March (Editors) 5th edition (January 2001) Wiley John & Sons; ISBN: 0471585890.

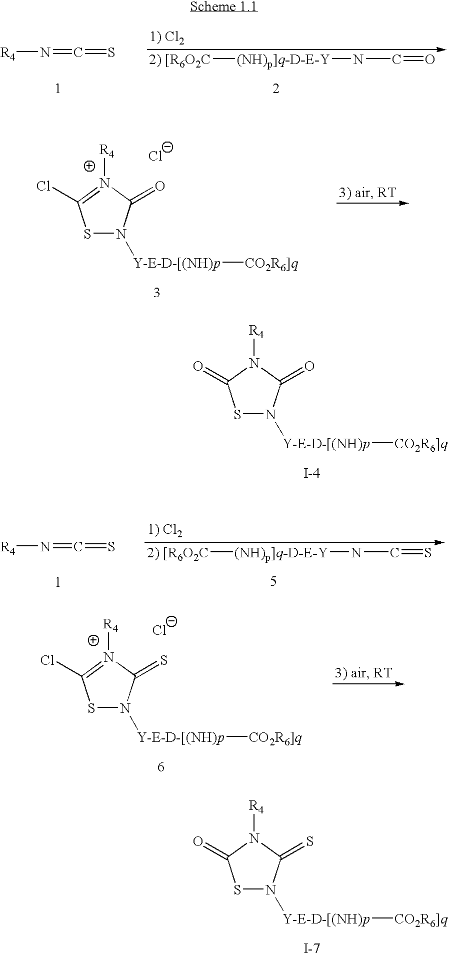

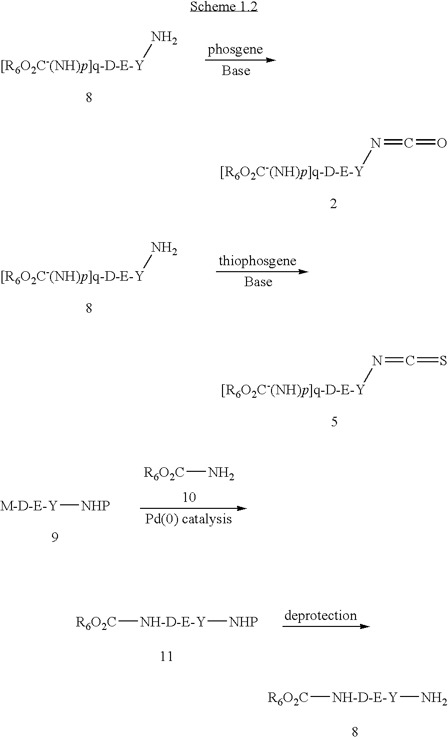

Compounds of Formula I wherein Q is taken from Q1 or Q-2 and Y is alkylene are also available via the synthetic route shown in Scheme 1.3. Reaction of amine 8 with isocyanate or isothiocyanate 2a yields the urea/thiourea 8a which can be cyclized by the addition of chlorocarbonyl sulfenyl chloride. See GB1115350 and U.S. Pat. No. 3,818,024, Revankar et. al U.S. Pat. No. 4,093,624, and Klayman et. al JOC 1972, 37(10), 1532 for further details. Where R₄ is a readily removable protecting group (e.g. R=3,4-d-methoxybenzyl amine), the action of mild, acidic deprotection conditions such as CAN or TFA will reveal the parent ring system of I-4 (X═O) and I-7 (X═S).

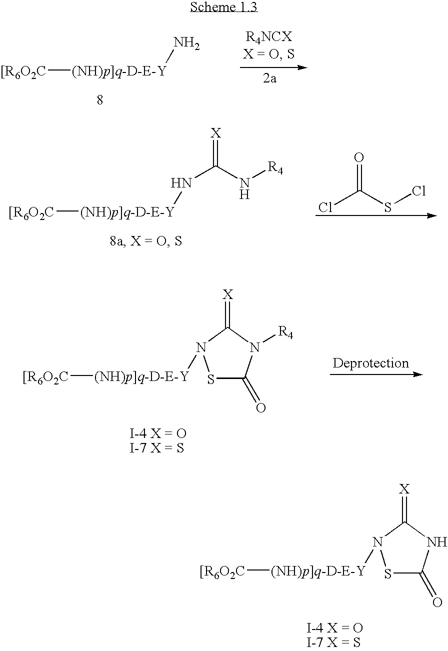

I-7 is also available as shown in Scheme 1.4. Condensation of isocyanate or isothiocyanate 2a with amine R₅NH₂ yields urea/thiourea 2b, which, when reacted with chlorocarbonyl sulfenyl chloride according to GB1115350 and U.S. Pat. No. 3,818,024 yields 2c. Where R₄ is a readily removable protecting group (e.g. R=3,4-d-methoxybenzyl amine), the action of mild, acidic deprotection conditions such as CAN or TFA will reveal the parent ring system of 2d. Reaction of 2d with NaH in DMF, and displacement wherein M is a suitable leaving group such as chloride, bromide or iodide yields I-4 (X═O) and I-7 (X═S).

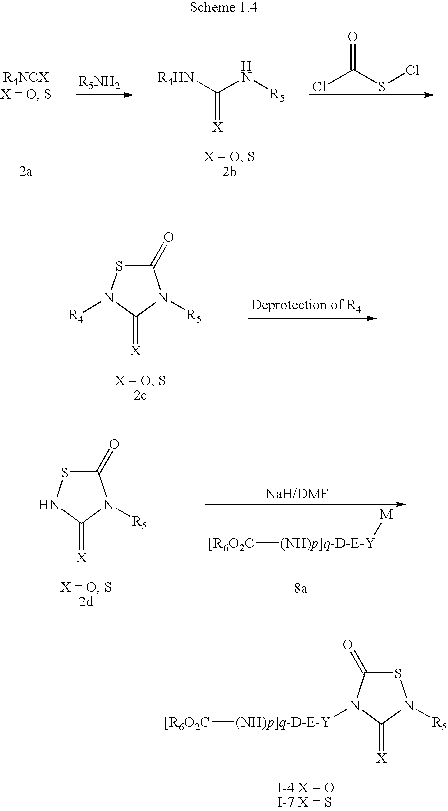

Compounds of Formula I wherein Q is taken from Q-1′ or Q-2′ and Y is alkylene are available via the synthetic route shown in Scheme 1.3. Condensation of isocyanate or isothiocyanate 2a with ammonia yields urea/thiourea 2e, which, when reacted with chlorocarbonyl sulfenyl chloride according to GB1115350 and U.S. Pat. No. 3,818,024 yields 2f. Reaction of 2f with NaH in DMF, and displacement wherein M is a suitable leaving group such as chloride, bromide or iodide yields yields I-4′ (X═O) and I-7′ (X═S).

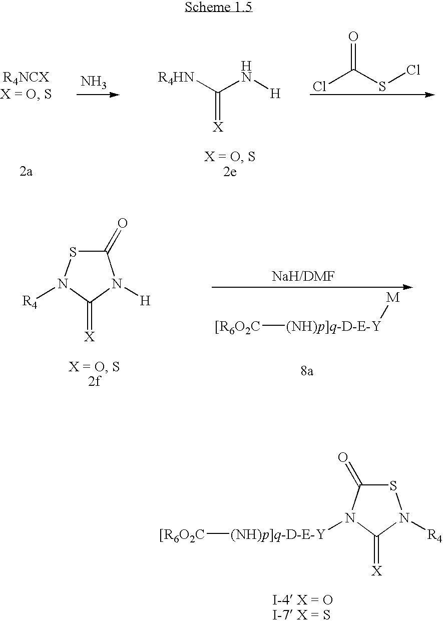

Compounds of Formula I wherein Q is taken from Q-3 or Q-4 and Y is alkylene, are prepared according to the synthetic route shown in Schemes 2.1 and 2.2, respectively. Reaction of 12, wherein M is a suitable leaving group, with the carbamate-protected hydrazine 13 affords intermediate 14. Reaction of 14 with an isocyanate gives rise to intermediate 15. Thermal cyclization of 15 affords 1,2,4-triazolidinedione of Formula I-16. By analogy, scheme 2.2 illustrates the preparation of 3-thio-5-oxo-1,2,4-triazolidines of Formula I-18 by reaction of intermediate 14 with an isothiocyanate and subsequent thermal cyclization.

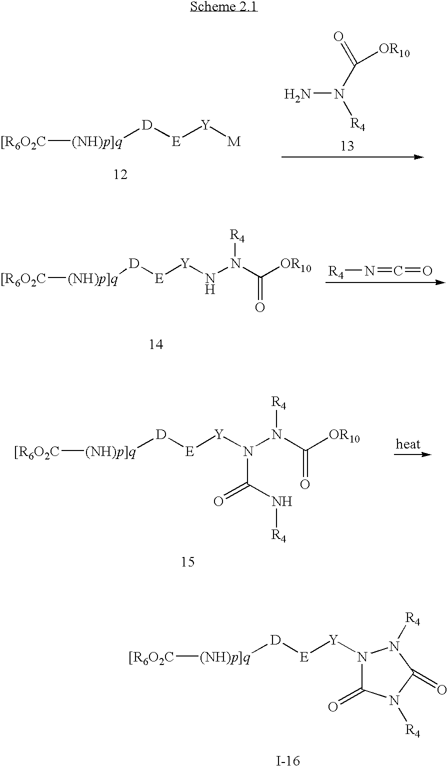

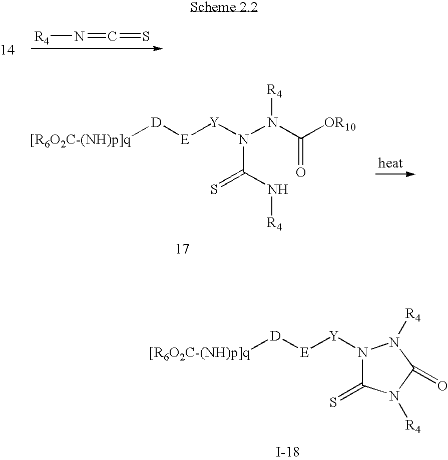

Intermediates 12 wherein p is 1 are readily available or are prepared by reaction of 19 with carbamates 10 under palladium(0)-catalyzed conditions. M₁ is a group which oxidatively inserts palladium(0), preferably iodo or bromo, and is of greater reactivity than M. Compounds 19 are either commercially available or prepared by one of ordinary skill in the art. 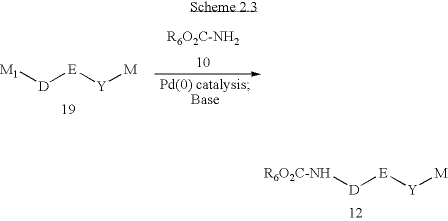
Compounds of Formula I wherein D is taken from Q-3 or Q-4 and Y is alkylene, are also prepared according to the synthetic route shown in Scheme 2.4. Oxidation of amine R₄NH₂ to the corresponding hydrazine, condensation with ethyl chloroformate subsequent heating yields 1,2,4-triazolidinedione 15a. After the action of NaH in DMF, displacement wherein M is a suitable leaving group such as chloride, bromide or iodide yields I-16 (X═O) and I-18 (X═S).

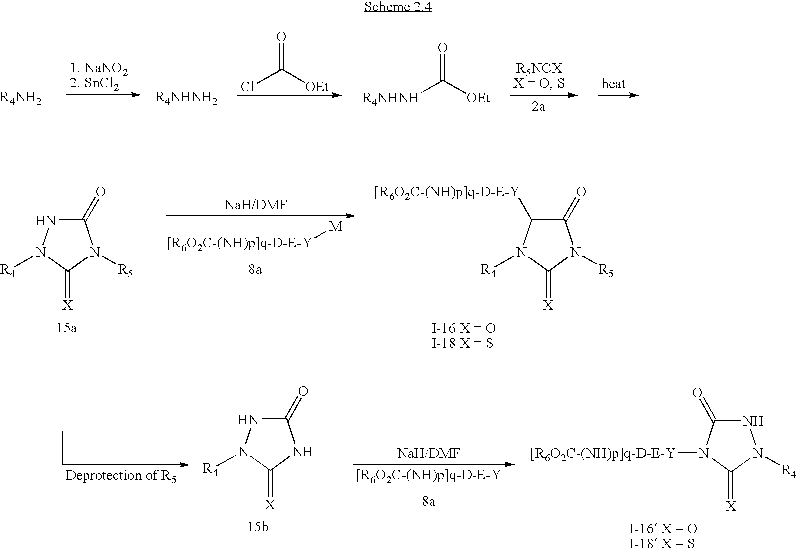

Compounds of Formula I wherein D is taken from D-3′ or D-4′ and Y is alkylene, are also prepared according to the synthetic route shown in Scheme 2.4. When R₅ is a readily removable protecting group (e.g. R=3,4-d-methoxybenzyl amine), the action of mild, acidic deprotection conditions such as CAN or TFA on 15a will reveal 1,2,4-triazolidinedione 15b. After deprotonation of 15b by NaH in DMF, displacement wherein M is a suitable leaving group such as chloride, bromide or iodide yields I-16′ (X═O) and I-18′ (X═S).

Compounds of Formula I wherein Q is taken from Q-5 or Q-6 and Y is alkylene are prepared according to the synthetic route shown in Scheme 3. Reaction of hydrazine 20 with chlorosulfonylisocyanate and base, such as triethylamine, gives rise to a mixture of intermediates 21A and 21B which are not isolated but undergo cyclization in situ to afford compounds of Formulae I-22A and I-22B. Compounds I-22A and I-22B are separated by chromatography or fractional crystallization. Optionally, compounds I-22A and I-22B can undergo Mitsunobu reaction with alcohols R₄OH to give compounds of Formulae I-23A and I-23B. Compounds 20 are prepared by acid-catalyzed deprotection of t-butyl carbamates of structure 14, wherein R₁₀ is t-butyl.

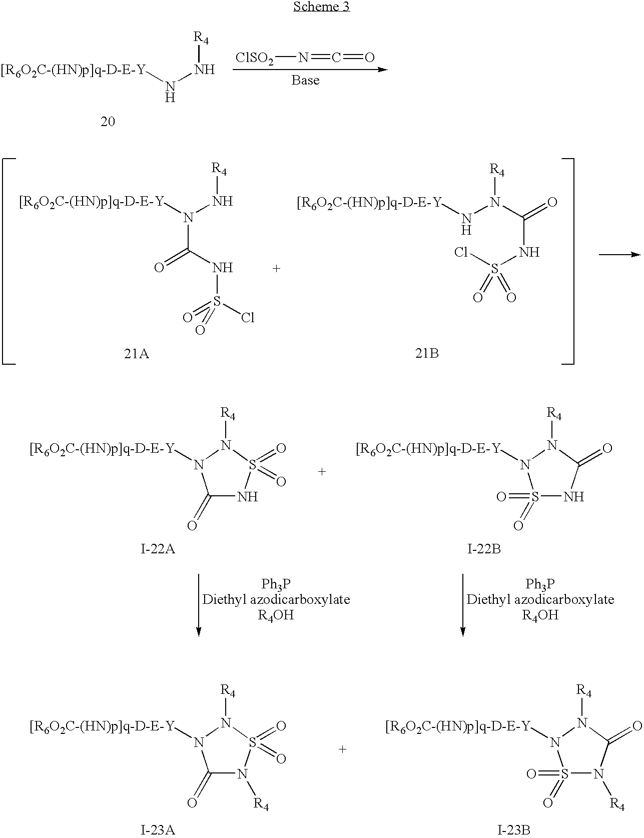

Compounds of Formula I wherein Q is Q-7 and Y is alkylene are prepared as shown in Scheme 4. Reaction of amine 8 with maleimide 24, wherein M is a suitable leaving group, affords compounds of Formula I-25. Reaction of compound 26, wherein M is a group which can oxidatively insert Pd(0), can participate in a Heck reaction with maleimide 27, affording compounds of Formula I-28. Maleimides 24 and 27 are commercially available or prepared by one of ordinary skill in the art.

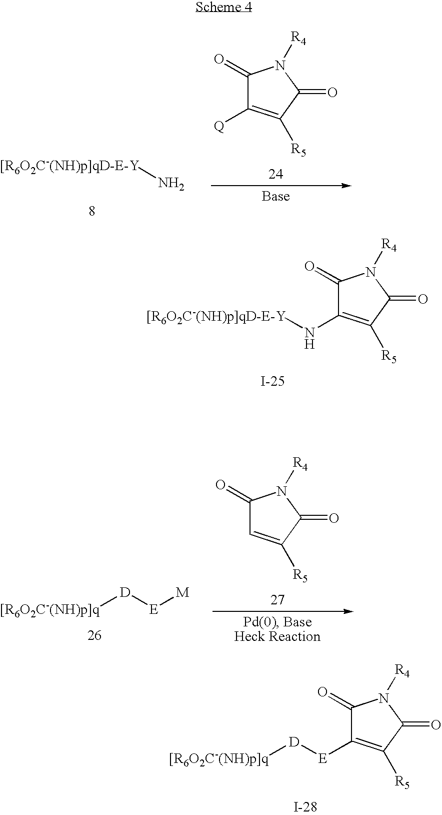

Compounds of Formula I wherein Q is Q-8 and Y is alkylene are prepared as shown in Scheme 5, according to methods reported by M. Tremblay et al, Journal of Combinatorial Chemistry (2002) 4:429. Reaction of polymer-bound activated ester 29 (polymer linkage is oxime activated-ester) with chlorosulfonylisocyante and t-butanol affords N—BOC sulfonylurea 30. Subjection of 30 to the Mitsunobu reaction with R₄OH gives rise to 31. BOC-group removal with acid, preferably trifluoroacetic acid, and then treatment with base, preferably triethylamine, provides the desired sulfahydantoin I-32. Optionally, intermediate 30 is treated with acid, preferably trifluoroacetic acid, to afford the N-unsubstituted sulfahydantoin I-33.

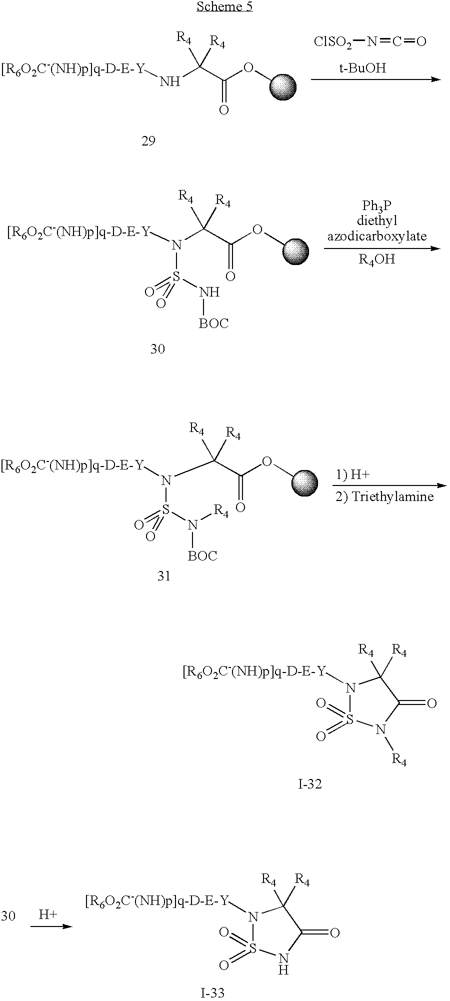

Compounds of Formula I wherein Q is Q-8 and Y is alkylene are also prepared as shown in Scheme 5a. Amine 8 is condensed with the glyoxal hemiester to yield 31a. Reaction of chlorosulphonyl isocyanate first with benzyl alcohol then 31a yields 31b, which after heating yields I-32.

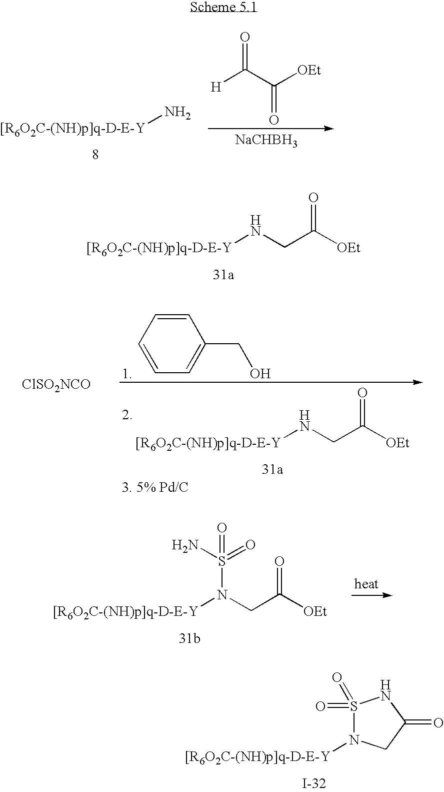

Compounds of Formula I wherein Q is taken from Q-8′, are prepared according to the synthetic route shown in Scheme 5.2. Formation of 31c by the method of Muller and DuBois JOC 1989, 54, 4471 and its deprotonation with NaH/DMF or NaH/DMF and subsequently alkylation wherein M is a suitable leaving group such as chloride, bromide or iodide yields I-32′. Alternatively, I-32′ is also available as shown in Scheme 5.3. Mitsunobu reaction of boc-sulfamide amino ethyl ester with alcohol 8b (made by methods analogous to that for amine 8) yields-31c, which after Boc removal with 2N HCl in dioxane is cyclized by the action of NaH on 31d results in I-32′.

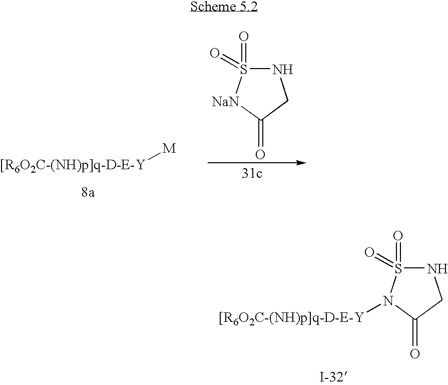

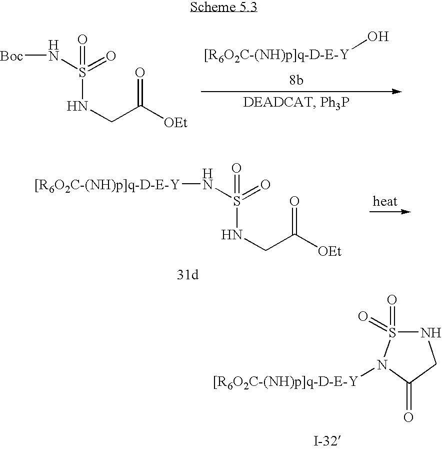

Compounds of Formula I wherein Q is Q-9 and Y is alkylene are prepared as shown in Scheme 6. Reaction of polymer-bound amino acid ester 34 with an isocyanate affords intermediate urea 35. Treatment of 35 with base, preferably pyridine or triethylamine, with optional heating, gives rise to compounds of Formula I-36.

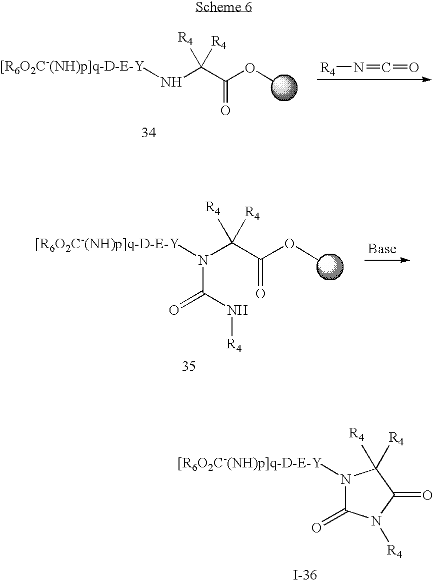

Compounds of Formula I wherein Q is Q-9 and Y is alkylene are also prepared as shown in Scheme 6.1. Reaction of aldehyde 8c under reductive amination conditions with the t-butyl ester of glycine yields 35a. Isocyanate 2a is condensed with p-nitrophenol (or the corresponding R₄NH₂ amine is condensed with p-nitrophenyl chloroformate) to yield the carbamic acid p-nitrophenyl ester, which when reacted with deprotonated 35a and yields the urea that when deprotected with acid yields 35b. Formula I-36 is directly available from 35b by the action of NaH and heat.

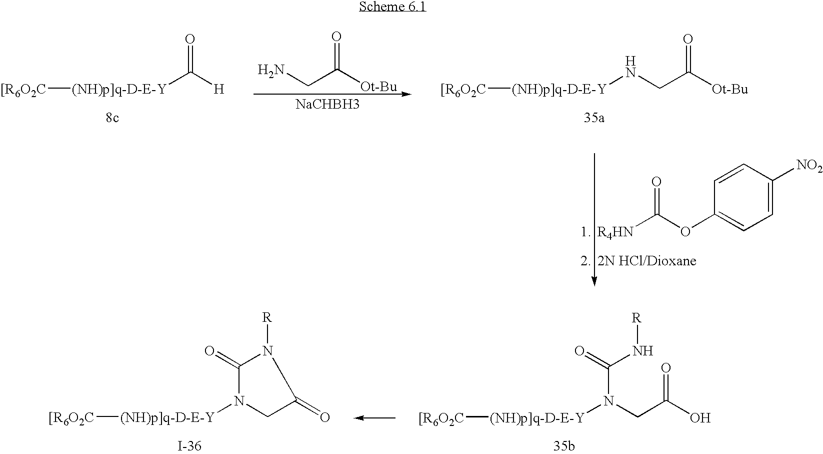

Compounds of Formula I wherein Q is taken from Q-9′, are prepared according to the synthetic route shown in Scheme 6.2. Formation of 35c by the method described in JP10007804A2 and Zvilichovsky and Zucker, Israel Journal of Chemistry, 1969, 7(4), 547-54 and its deprotonation with NaH/DMF or NaH/DMF and its subsequent displacement of M, wherein M is a suitable leaving group such as chloride, bromide or iodide, yields I-36′.

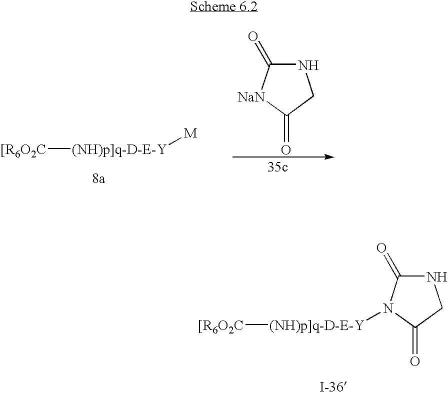

Compounds of Formula I wherein Q is Q-10 or Q-11, and Y is alkylene are prepared as shown in Schemes 7.1 and 7.2, respectively. Treatment of alcohol 37 (Z=O) or amine 37 (Z=NH) with chlorosulfonylisocyanate affords intermediate carbamate or urea of structure 38. Treatment of 38 with an amine of structure HN(R₄)₂ and base, preferably triethylamine or pyridine, gives sulfonylureas of Formula I-39. Reaction of chlorosulonylisocyanate with an alcohol (Z=O) or amine (Z=NR₄) 40 affords intermediate 41. Treatment of 41 with an amine 8 and base, preferably triethylamine or pyridine, gives sulfonylureas of Formula I-42.

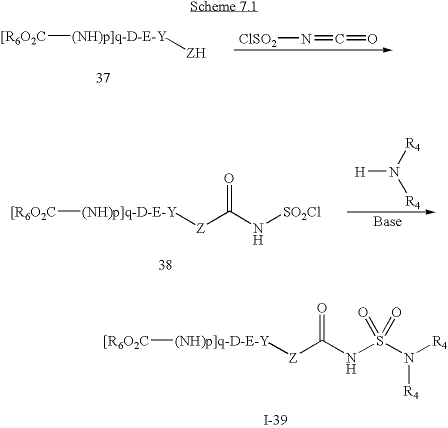

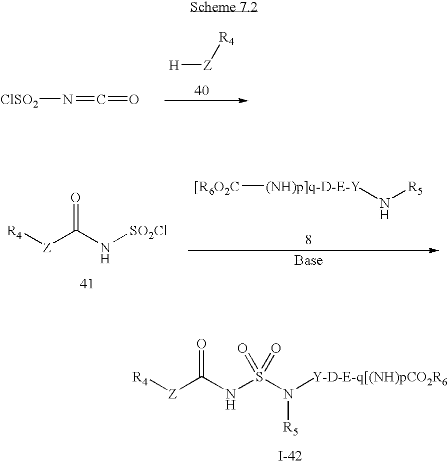

Compounds of Formula I wherein Q is taken from Q-12 are prepared according to the synthetic route shown in Scheme 8. Alkylation of pyridine 43, wherein TIPS is tri-isopropylsilyl, under standard conditions (K₂CO₃, DMF, R₄—I or Mitsunobu conditions employing R₄—OH) yields pyridine derivative 44 which is reacted with compound 12, wherein M is a suitable leaving group, to afford pyridones of formula I-45.

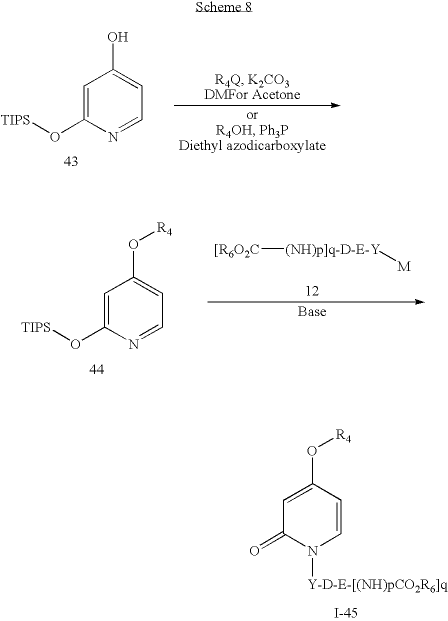

Compounds of Formula I wherein Q is taken from Q-13 are prepared according to the synthetic route shown in Scheme 9. Starting from readily available pyridine 46, alkylation under standard conditions (K₂CO₃, DMF, R₄—I or Mitsunobu conditions employing R₄—OH) yields pyridine derivative 47. N-alkylation with K₂CO₃, DMF, R₄—I affords pyridones of formula 48. Intermediate 48 is partitioned to undergo a Heck reaction, giving I-49; a Buchwald amination reaction, giving I-51; or a Buchwald Cu(I) catalyzed O-arylation reaction, to give I-52. The Heck reaction product I-49 may be optionally hydrogenated to afford the saturated compound I-50. Wherein the phenyl ether R group is methyl, compounds of formula I-49, I-50, I-51, or I-52 are treated with boron tribromide or lithium chloride to afford compounds of Formula I-53, wherein R₄ is hydrogen.

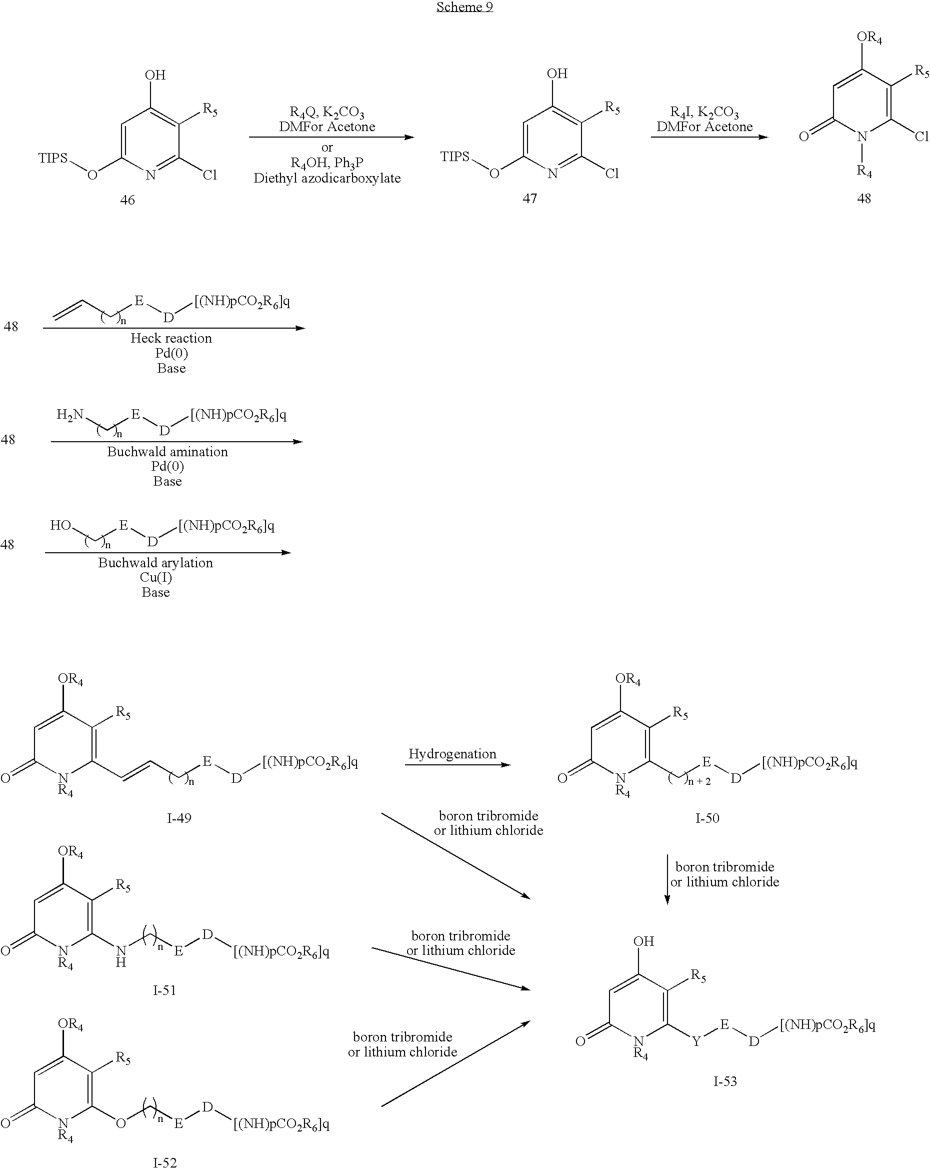

Compounds of Formula I wherein Q is taken from Q-14 are prepared according to the synthetic route shown in Scheme 10. Starting from readily available pyridine 54, alkylation under standard conditions (K₂CO₃, DMF, R₄—I or Mitsunobu conditions employing R₄—OH) yields pyridine derivative 55. N-alkylation with K₂CO₃, DMF, R₄—I affords pyridones of formula 56. Intermediate 56, wherein M is a suitable leaving group, preferably bromine or chlorine, is partitioned to undergo a Heck reaction, giving I-57; a Buchwald amination reaction, giving I-59; or a Buchwald Cu(I) catalyzed O-arylation reaction, to give I-60. The Heck reaction product I-57 may be optionally hydrogenated to afford the saturated compound I-58. Wherein R₄ is methyl, compounds of formula I-57, I-58, I-59, or I-60 are treated with boron tribromide or lithium chloride to afford compounds of Formula I-61, wherein R₄ is hydrogen.

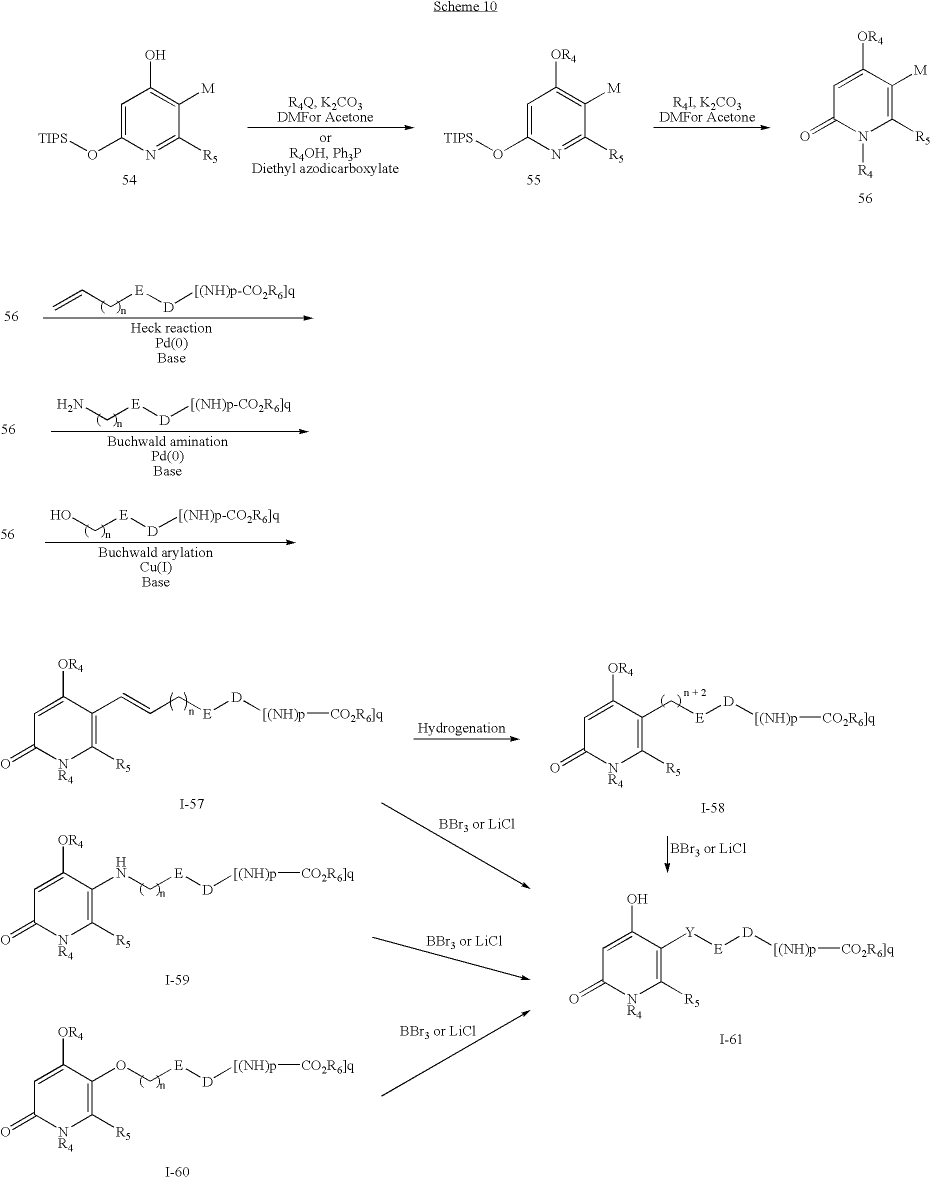

Compounds of Formula I wherein Q is taken from Q-15 are prepared according to the synthetic routes shown in Schemes 11 and 12. Starting esters 62 are available from the corresponding secoacids via TBS-ether and ester formation under standard conditions. Reaction of protected secoester 62 with Meerwin't salt produces the vinyl ether 63 as a pair of regioisomers. Alternatively, re action of 62 with dimethylamine affords the vinylogous carbamate 64. Formation of the dihydropyrimidinedione 66 proceeds by condensation with urea 65 with azeotropic removal of dimethylamine or methanol. Dihydropyrimidinedione 66 may optionally be further substituted by Mitsunobu reaction with alcohols R₄OH to give rise to compounds 67.

Scheme 12 illustrates the further synthetic elaboration of intermediates 67. Removal of the silyl protecting group (TBS) is accomplished by treatment of 67 with flouride (tetra-n-butylammonium fluoride or cesium flouride) to give primary alcohols 68. Reaction of 68 with isocyanates 2 gives rise to compounds of Formula I-69. Alternatively, reaction of 68 with [R₆O₂C(NH)p]q-D-E-M, wherein M is a suitable leaving group, affords compounds of Formula I-70. Oxidation of 68 using the Dess-Martin periodinane (D. Dess, J. Martin, J. Am. Chem. Soc. (1991) 113:7277) or tetra-n-alkyl peruthenate (W. Griffith, S. Ley, Aldrichimica Acta (1990) 23:13) gives the aldehydes 71. Reductive amination of 71 with amines 8 gives rise to compounds of Formula I-72. Alternatively, aldehydes 71 may be reacted with ammonium acetate under reductive alkylation conditions to give rise to the primary amine 73. Reaction of 73 with isocyanates 2 affords compounds of Formula I-74.

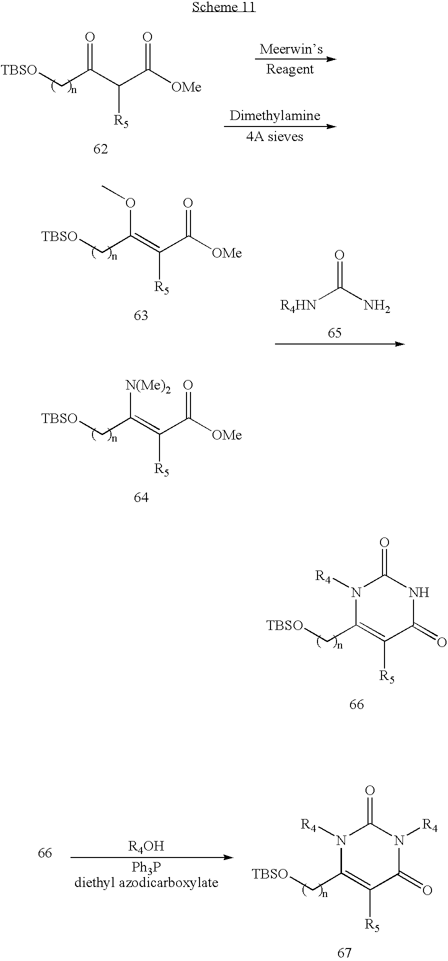

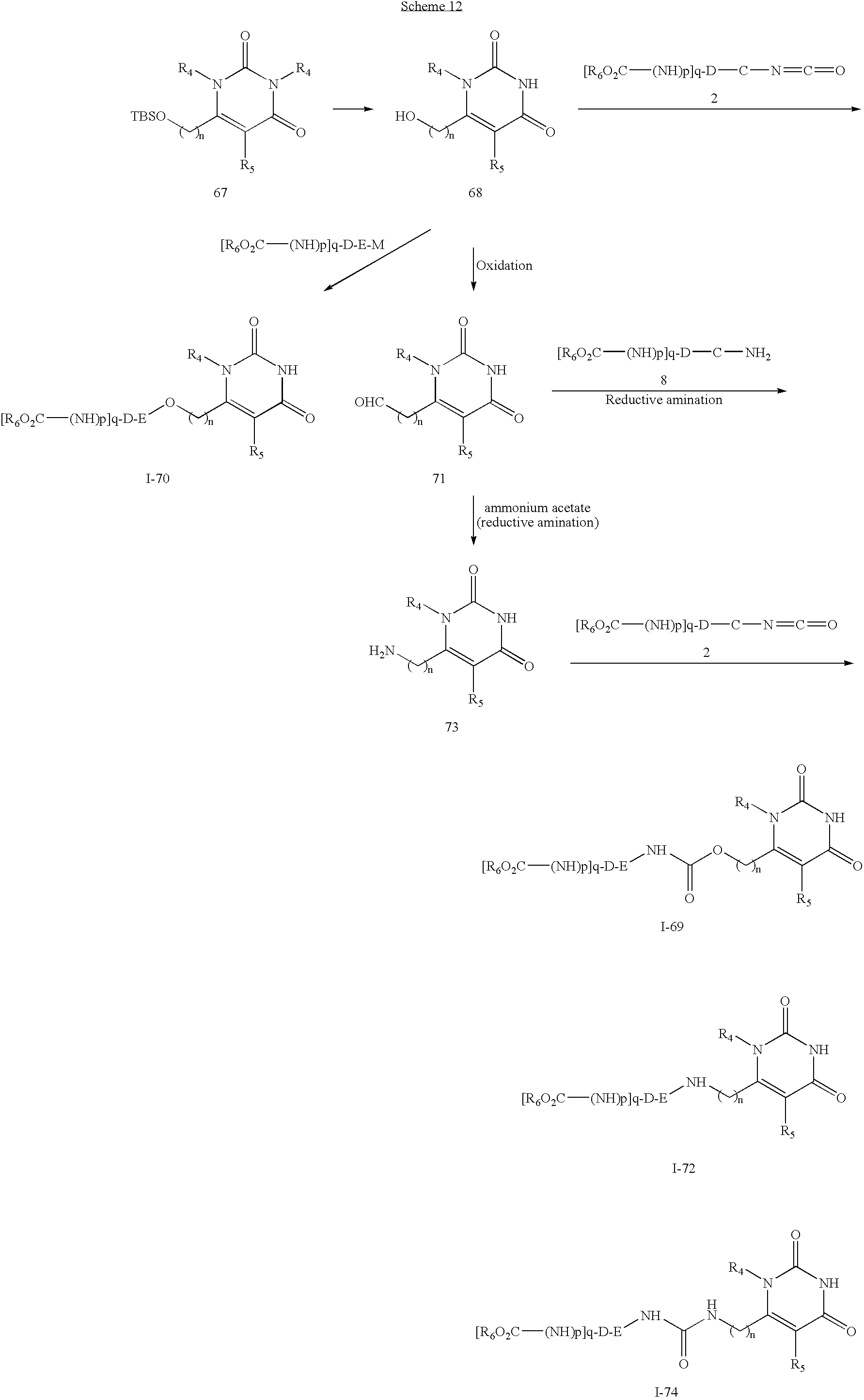

Compounds of Formula I wherein Q is taken from Q-16 are prepared according to the synthetic routes shown in Schemes 13 and 14. Starting esters 75 are available from the corresponding secoacids via TBS-ether and ester formation under standard conditions. Reaction of protected secoester 75 with Meerwin's salt produces the vinyl ether 76 as a pair of regioisomers. Alternatively, reaction of 75 with dimethylamine affords the vinylogous carbamate 77. Formation of the dihydropyrimidinedione 78 proceeds by condensation with urea 65 with azeotropic removal of dimethylamine or methanol. Dihydropyrimidinedione 78 may optionally be further substituted by Mitsunobu reaction with alcohols R₄OH to give rise to compounds 79. Compounds of Formulae I-81, I-82, I-84, and I-86 are prepared as shown in Scheme 14 by analogy to the sequence previously described in Scheme 12.

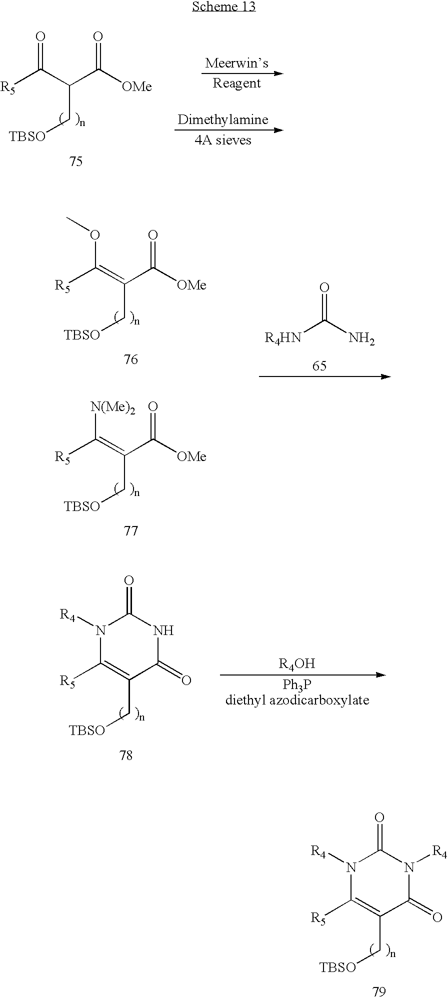

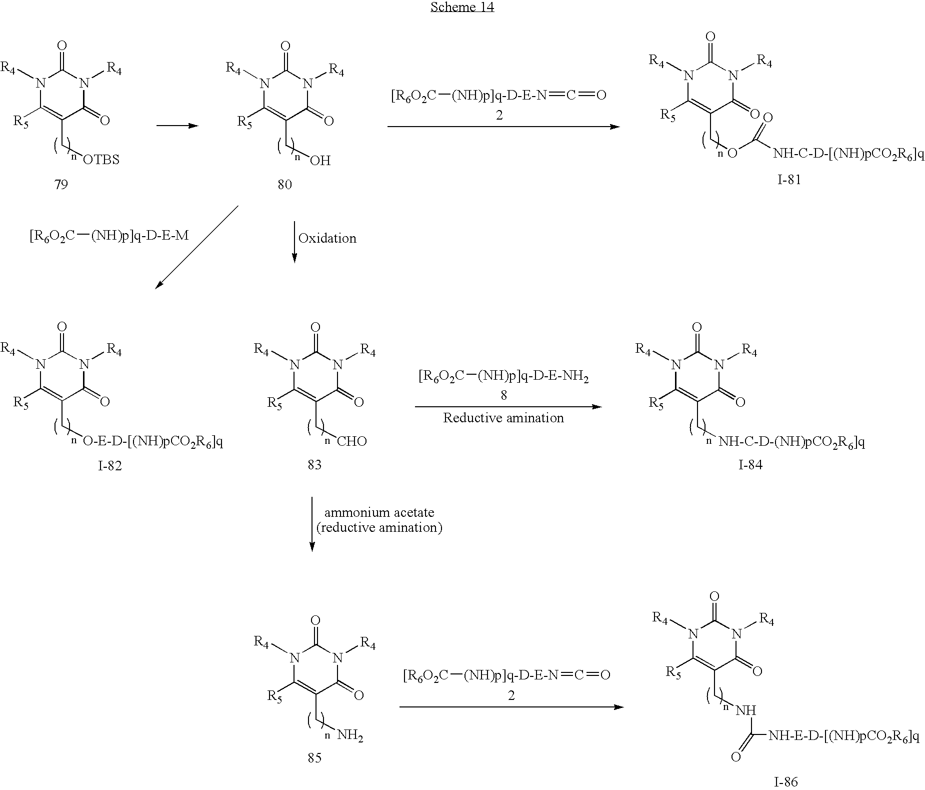

Alkyl acetoacetates 87 are commercially available and are directly converted into the esters 88 as shown in Scheme 15. Treatment of 87 with NaHMDS in THF, followed by quench with formaldehyde and TBSCl (n=1) or Q-(CH2)n-OTBS (n=2-4), gives rise to compounds 88.

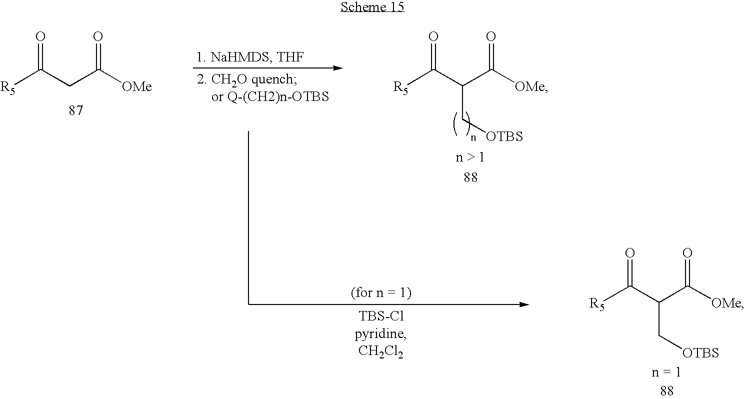

Compounds of Formula I wherein Q is taken from Q-17 are prepared according to the synthetic routes shown in Schemes 16.1 and 16.2, and starts with the BOC-protected hydrazine 13, which is converted to the 1,2-disubstituted hydrazine 89 by a reductive alkylation with a glyoxal derivative mediated by sodium cyanoborohydride and acidic workup. Condensation of 89 with diethyl malonate in benzene under reflux yields the heterocycle 90. Oxidation with N₂O₄ in benzene (see Cardillo, Merlini and Boeri Gazz. Chim. Ital., (1966) 9:8) to the nitromalonohydrazide 91 and further treatment with P₂O₅ in benzene (see: Cardillo, G. et al, Gazz. Chim. Ital. (1966) 9:973-985) yields the tricarbonyl 92. Alternatively, treatment of 90 with Brederick's reagent (t-BuOCH(N(Me₂)₂, gives rise to 93, which is subjected to ozonolysis, with a DMS and methanol workup, to afford the protected tricarbonyl 92. Compound 92 is readily deprotected by the action of CsF in THF to yield the primary alcohol 94. Alcohol 94 is optionally converted into the primary amine 95 by a sequence involving tosylate formation, azide displacement, and hydrogenation.

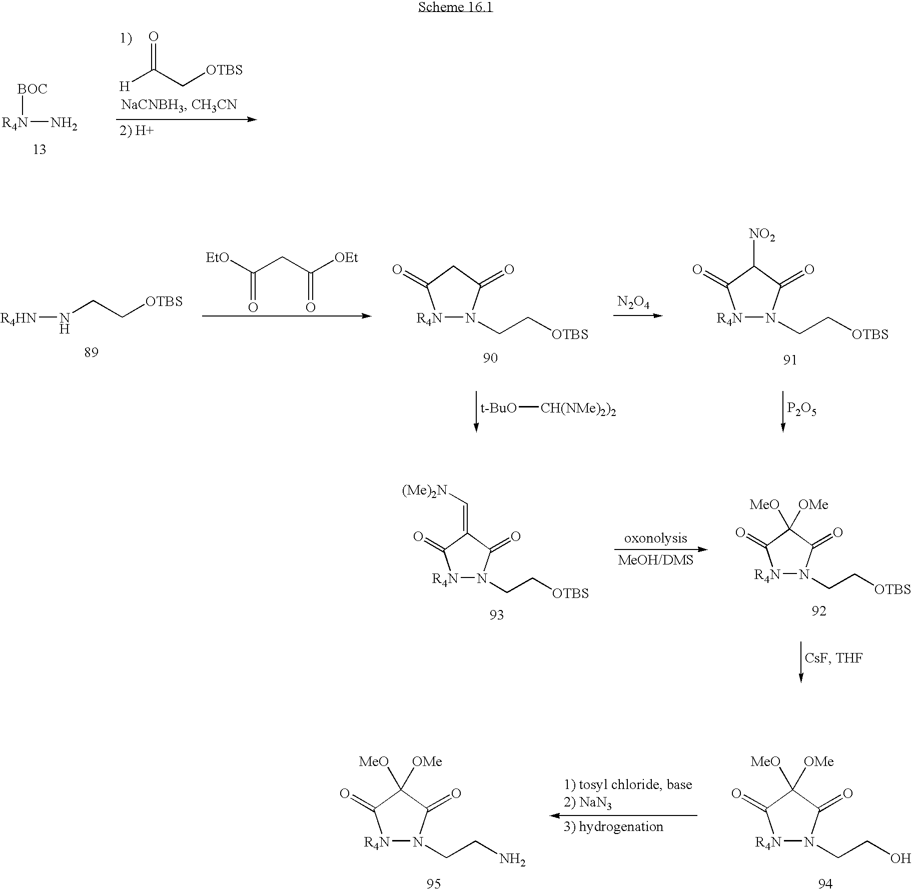

Reaction of 94 with (hetero)aryl halide 26, wherein M is iodo, bromo, or chloro, under copper(I) catalysis affords compounds I-96. Optional deprotection of the di-methyl ketal with aqueous acid gives rise to compounds of Formula I-98. By analogy, reaction of amine 95 with 26 under palladium(0) catalysis affords compounds of Formula I-97. Optional deprotection of the di-methyl ketal with aqueous acid gives rise to compounds of Formula I-99.

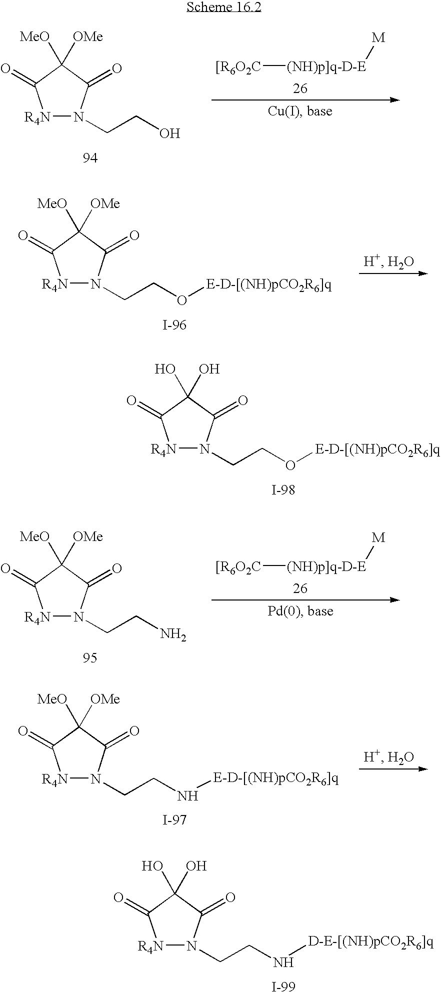

Compounds of Formula I wherein Q is taken from Q-17 are also prepared according to the synthetic route shown in Scheme 16.3. Deprotonation of 4,4-dimethyl-3,5-dioxo-pyrazolidine (95a, prepared according to the method described in Zinner and Boese, D. Pharmazie 1970, 25(5-6), 309-12 and Bausch, M. J. et. al J. Org. Chem. 1991, 56(19), 5643) with NaH/DMF or NaH/DMF and its subsequent displacement of M, wherein M is a suitable leaving group such as chloride, bromide or iodide yields I-99a.

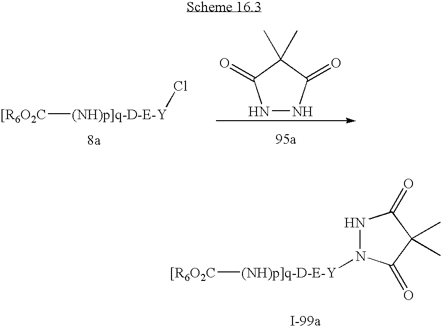

Compounds of Formula I wherein Q is taken from Q-18 are prepared as shown in Schemes 17.1 and 17.2. Aminoesters 100 are subjected to reductive alkylation conditions to give rise to intermediates 101. Condensation of amines 101 with carboxylic acids using an acid activating reagent such as dicyclohexylcarbodiimide (DCC)/hydroxybenzotriazole (HOBt) affords intermediate amides 102. Cyclization of amides 102 to tetramic acids 104 is mediated by Amberlyst A-26 hydroxide resin after trapping of the in situ generated alkoxide 103 and submitting 103 to an acetic acid-mediated resin-release.

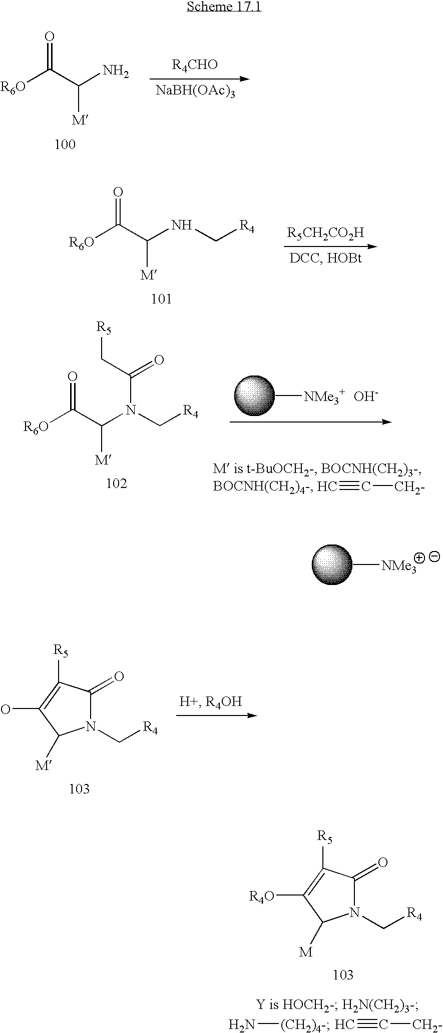

Scheme 17.2 illustrates the synthetic sequences for converting intermediates 104 to compounds of Formula I. Reaction of alcohol 104.1 with aryl or heteroaryl halide 26 (Q=halogen) under copper(I) catalysis gives rise to compounds of Formula I-105.1. Reaction of amines 104.2 and 104.3 with 26 under Buchwald palladium(0) catalyzed amination conditions affords compounds of Formulae I-105.2 and I-105.3. Reaction of acetylene 104.4 with 26 under Sonogashira coupling conditions affords compounds of Formula I-105.4. Compounds I-105.4 may optionally be reduced to the corresponding saturated analogs I-105.5 by standard hydrogenation.

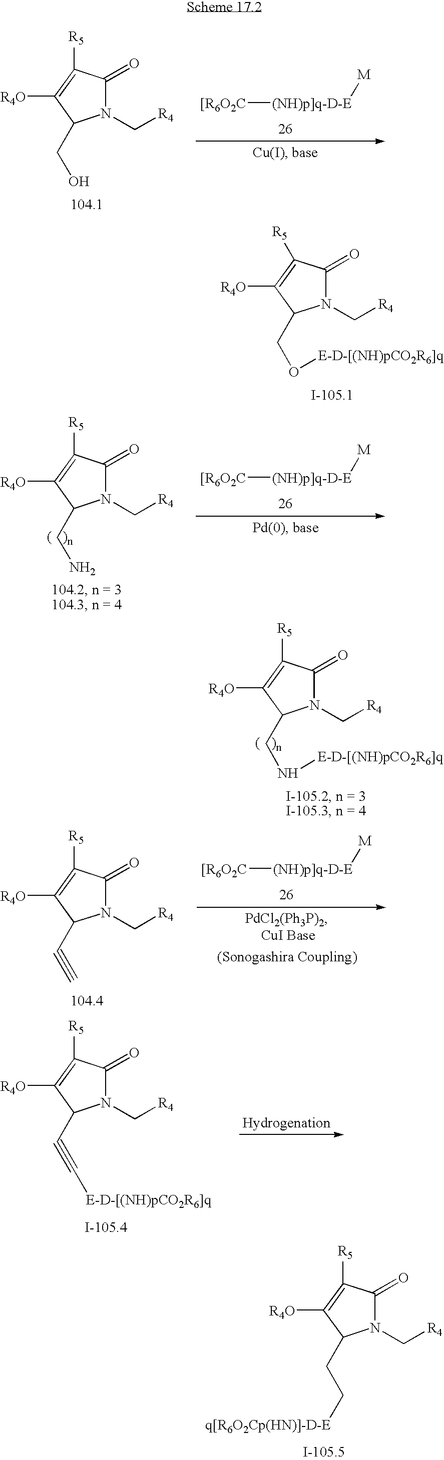

Compounds of Formula I wherein Q is taken from Q-19, Q-20, or Q-21 are prepared as illustrated in Scheme 18. Commercially available Kemp's acid 106 is converted to its anhydride 107 using a dehydrating reagent, preferably di-isopropylcarbodiimide (DIC) or 1-(3-dimethylaminopropyl)-3-ethylcarbodiimide (EDC). Reaction of 107 with amines R₄NH₂ affords the intermediate amides which are cyclized to the imides 108 by reaction with DIC or EDC. Alternatively, 107 is reacted with amines 8 to afford amides of Formula I-110. Amides I-110 may optionally be further reacted with DIC or EDC to give rise to compounds of Formula I-111. Acid 108 is further reacted with amines 8 to give compounds of Formula I-109.

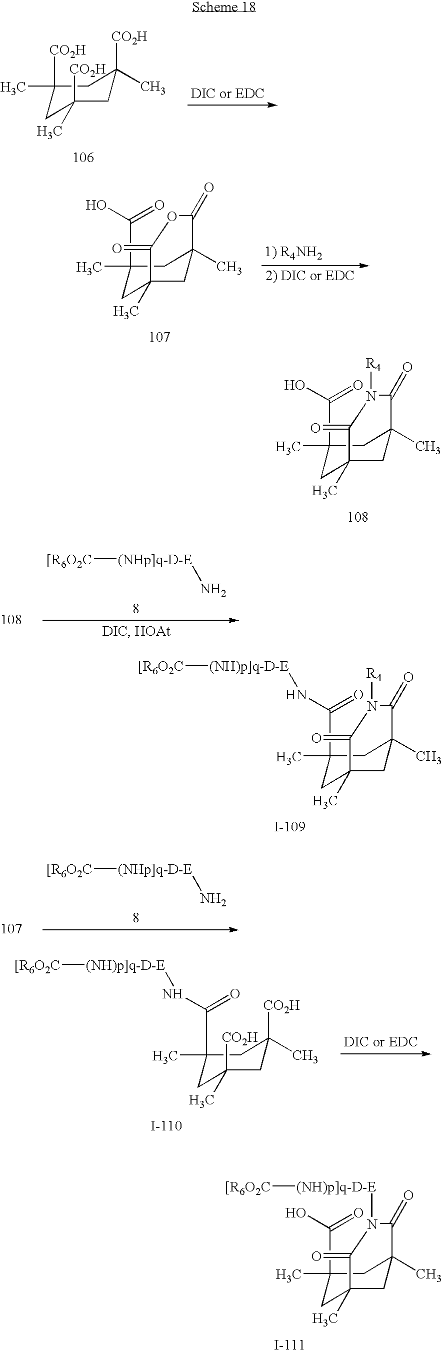

Compounds of Formula I wherein Q is taken from Q-22 or Q-23 are prepared as shown in Schemes 19.1 through 19.3. Preparation of intermediates 113 and 114 are prepared as shown in Scheme 19.1 from di-halo(hetero)aryls 112, wherein M₂ is a more robust leaving group than M₁. Reaction of 112 with amines 37 (Z=NH) either thermally in the presence of base or by palladium(0) catalysis in the presence of base and phosphine ligand affords compounds 113. Alternatively, reaction of 112 with alcohols 37 (X═O) either thermally in the presence of base or by copper(I) catalysis in the presence of base affords compounds 114.

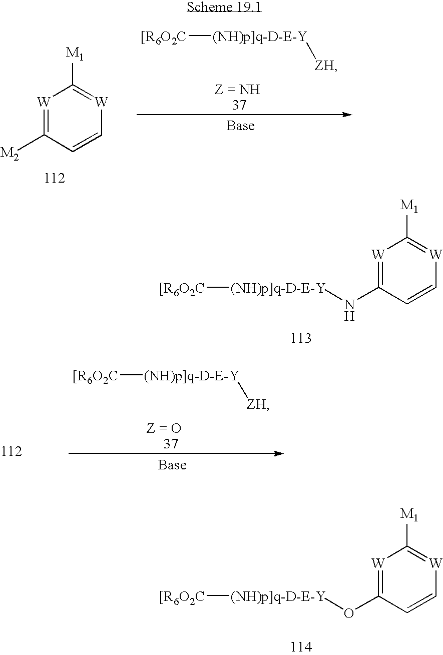

Scheme 19.2 illustrates the conversion of intermediates 113 into compounds of Formula I-115, I-118, or 117. Treatment of 113 with aqueous copper oxide or an alkaline hydroxide affords compounds of Formula I-115. Alternatively, treatment of 113 with t-butylmercaptan under copper(I) catalysis in the presence of ethylene glycol and potassium carbonate gives rise to 116 (see F. Y. Kwong and S. L. Buchwald, Organic Letters (2002) 4:3517. Treatment of the t-butyl sulfide 116 with acid affords the desired thiols of Formula I-118. Alternatively, 113 may be treated with excess ammonia under pressurized conditions to afford compound 117.

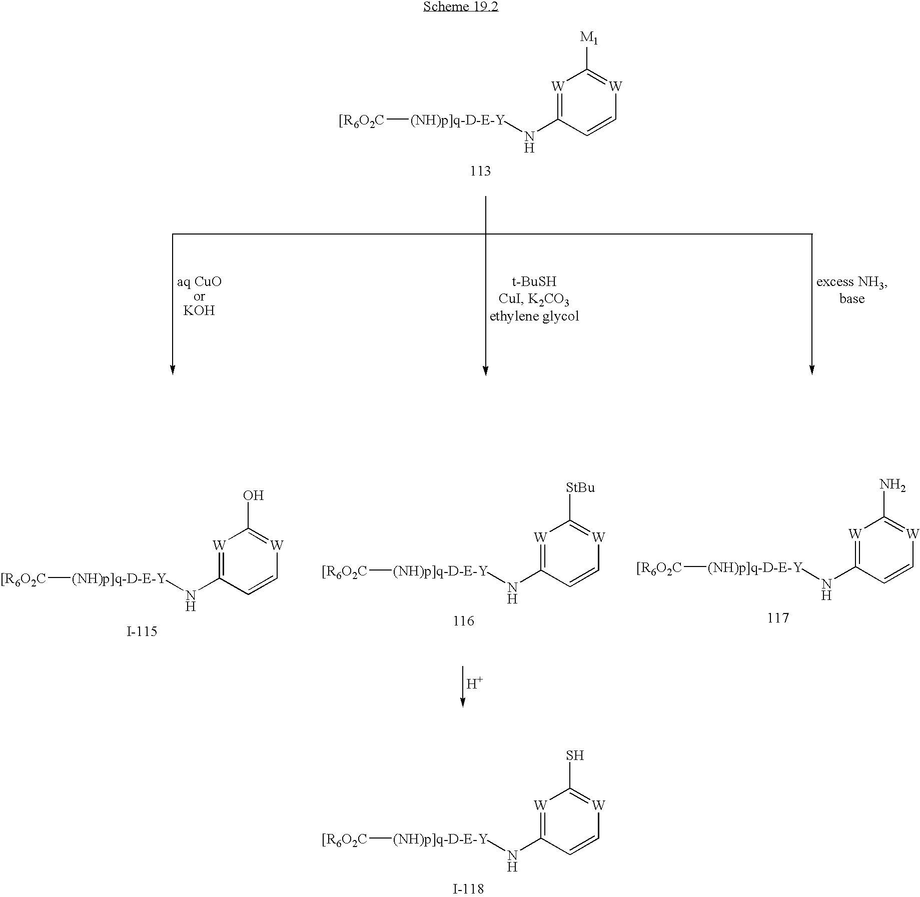

Scheme 19.3 illustrates the conversion of intermediate 114 into compounds of Formula I-119, I-122, and 121, by analogy to the sequence described in Scheme 19.2.

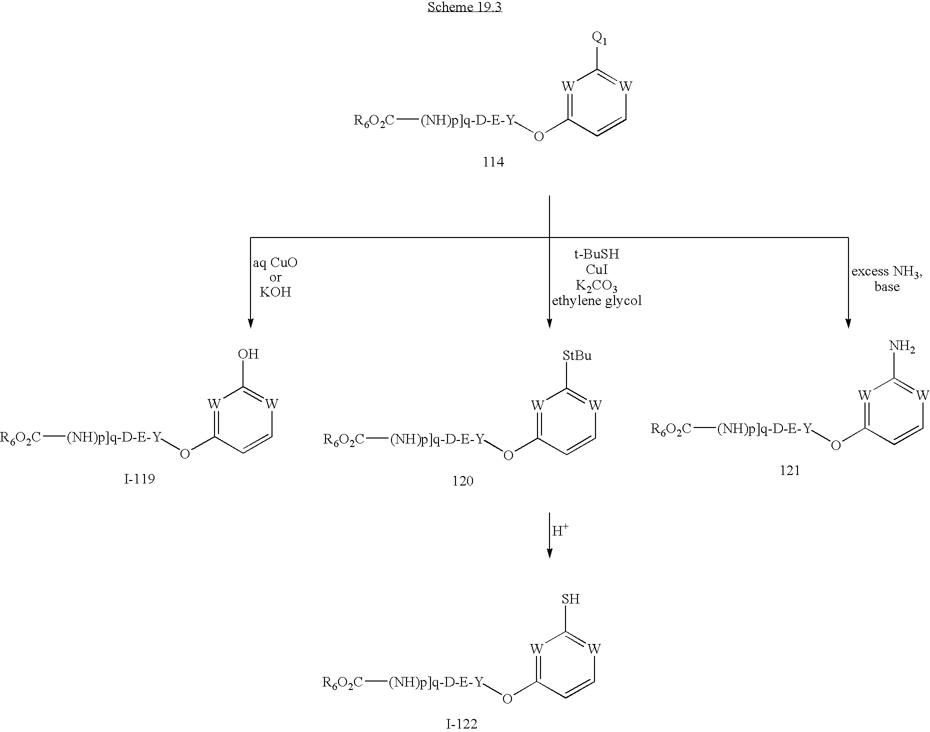

Compounds of Formula I wherein q is taken from Q-24, Q-25, or Q-26 are prepared as shown in Scheme 20. Reaction of compounds I-115 or I-119 with chlorosulfonylisocyanate, followed by in situ reaction with amines HN(R₄)₂ gives rise to compounds of Formulae I-123 or I-124. Reaction of compounds I-118 or I-122 with a peracid, preferably peracetic acid or trifluoroperacetic acid, affords compounds of Formula I-125 or I-126. Reaction of compounds 117 or 121 with chlorosulfonylisocyanate, followed by in situ reaction with amines HN(R₄)₂ or alcohols R₄OH, affords compounds of Formulae I-127, I-128, I-129, or I-130.

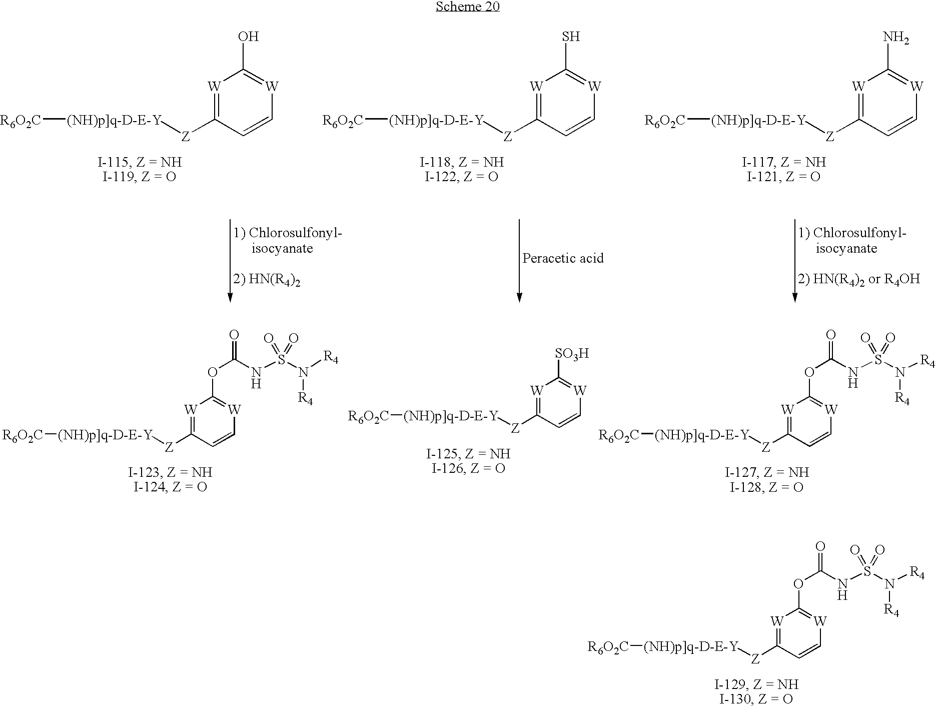

Compounds of Formula I wherein Q is taken from Q-27 are prepared as illustrated in Scheme 21. Reductive alkylation of thiomorpholine with aldehydes 131 affords benzylic amines 132, which are then subjected to peracid oxidation to give rise to the thiomorpholine sulfones 133 (see C. R. Johnson et al, Tetrahedron (1969) 25: 5649). Intermediates 133 are reacted with amines 8 (Z=NH₂) under Buchwald palladium-catalyzed amination conditions to give rise to compounds of Formula I-134. Alternatively, compounds 133 are reacted with alcohols 8 (Z=OH) under Buchwald copper(I) catalyzed conditions to afford compounds of Formula I-135. Alternatively, intermediates 133 are reacted with alkenes under palladium(0)-catalyzed Heck reaction conditions to give compounds of Formula I-136. Compounds I-136 are optionally reduced to the corresponding saturated analogs I-137 by standard hydrogenation conditions or by the action of diimide.

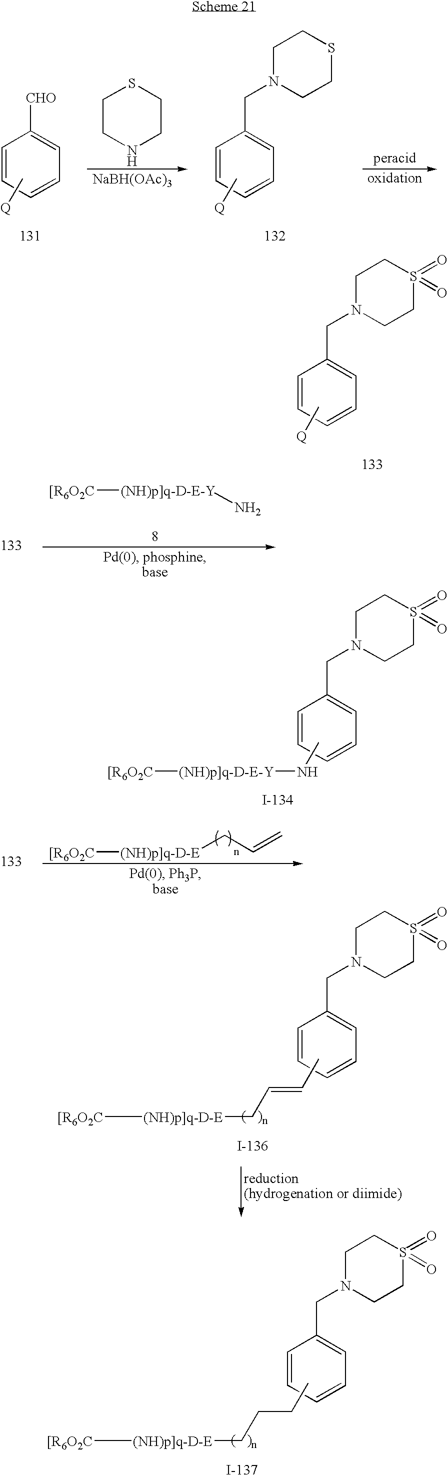

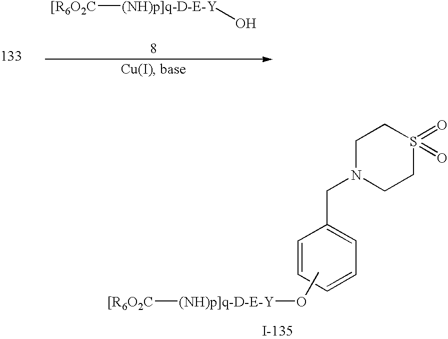

Compounds of Formula I wherein Q is taken from Q-27 are also prepared as illustrated in Scheme 21.1. Aldehyde 8c is reductively aminated with ammonia, and the resultant amine condensed with divinyl sulphone to yield I-134. Intermediate 134a is also available by reduction of amide 8d under a variety of standard conditions.

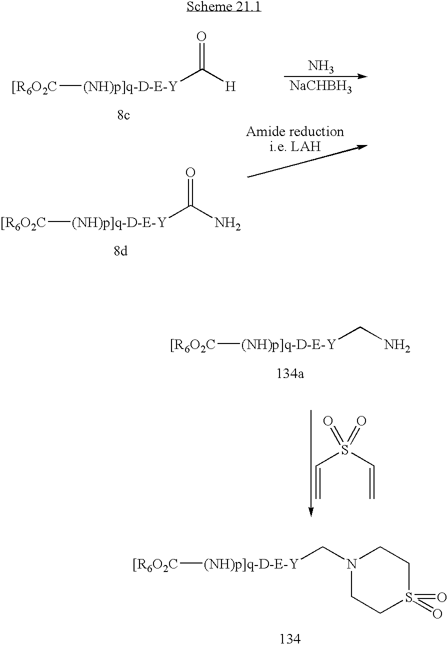

More generally, amines 134c are available via the reduction of amides 134b as shown in Scheme 21.2. The morpholine amide analogues 134d and morpholine analogues 134e are also available as shown in Scheme 21.2.

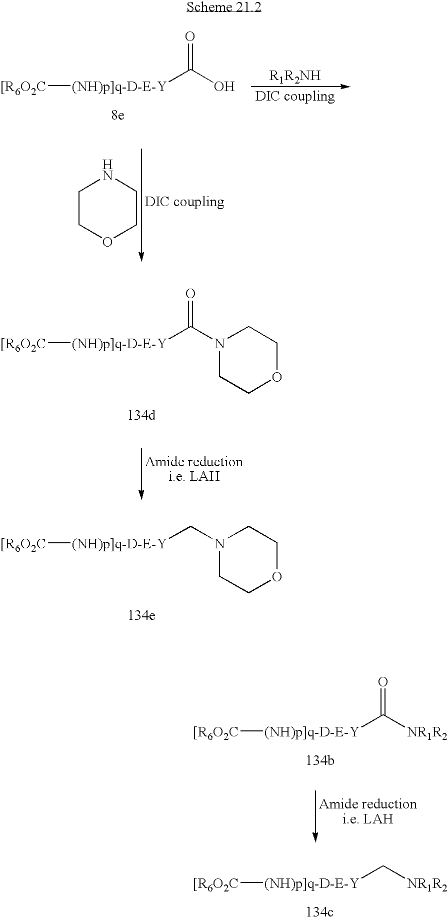

Compounds of Formula I wherein Q is taken from Q-28 or Q-29 are prepared according to the sequences illustrated in Scheme 22. Readily available amides 138 are reacted with chlorosulfonylisocyanate to give intermediates 140, which are reacted in situ with amines HN(R₄)₂ or alcohols R₄OH to afford compounds of Formulae I-141 or I-142, respectively. Alternatively, amides 138 are reacted with sulfonylchlorides to give compounds of Formula I-139.

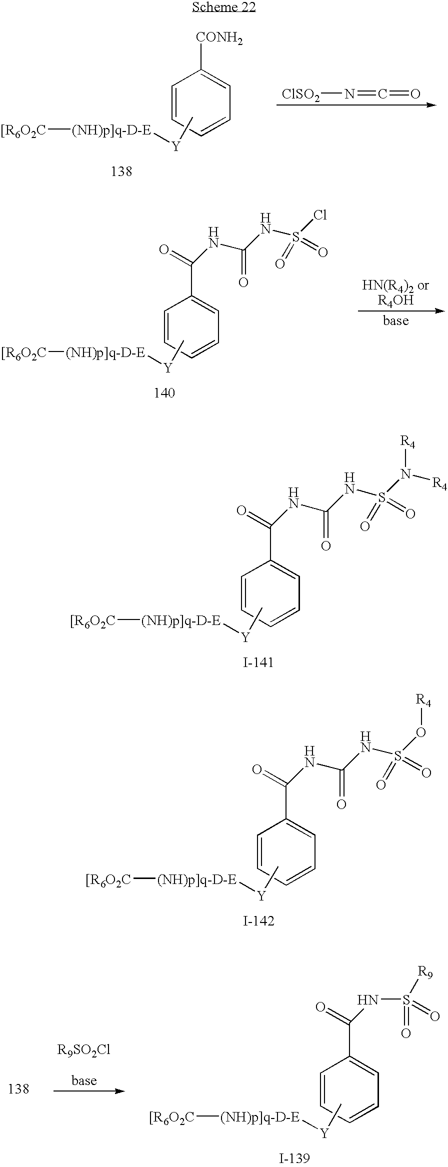

Compounds of Formula I wherein Q is taken from Q-30 are prepared as shown in Scheme 23. Readily available N—BOC anhydride 143 (see S. Chen et al, J. Am. Chem. Soc. (1996) 118:2567) is reacted with amines HN(R₄)₂ or alcohols R₆OH to afford acids 144 or 145, respectively. Intermediates 144 or 145 are further reacted with amines HN(R₄)₂ in the presence of an acid-activating reagent, preferably PyBOP and di-isopropylethylamine, to give diamides 146 or ester-amides 147. Intermediate 145 is converted to the diesters 148 by reaction with an alkyl iodide in the presence of base, preferably potassium carbonate. Intermediates 146-148 are treated with HCl/dioxane to give the secondary amines 149-151, which are then condensed with acids 152 in the presence of PyBOP and di-isopropylethylamine to give compounds of Formula I-153.

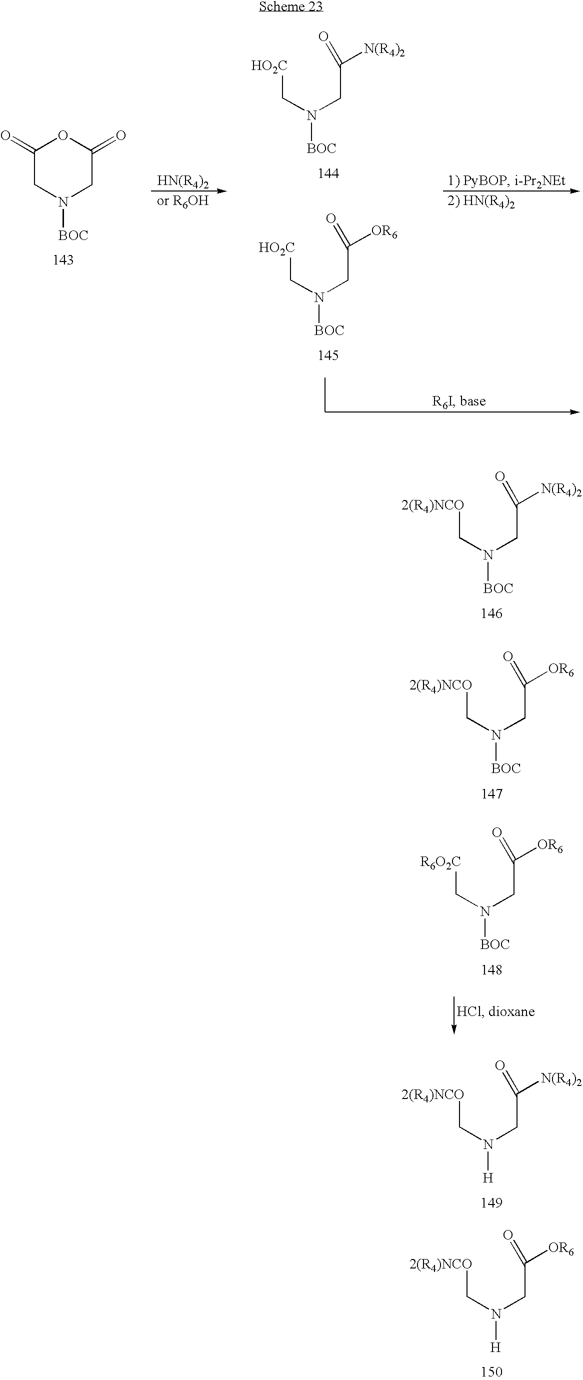

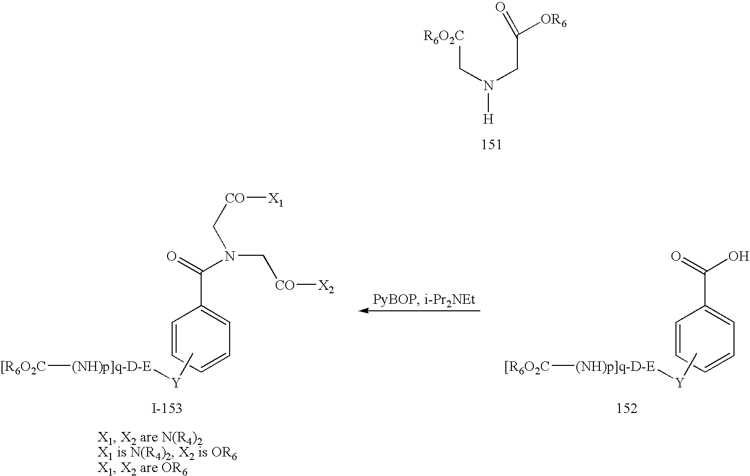

Compounds of Formula I wherein Q is taken from Q-31 or Q-32 are prepared according to the sequences illustrated in Scheme 24. Treatment of readily available sulfenamides 154 with amines 37 (Z=NH), alcohols 37 (Z=O), or alkenes 37 (Z=—CH═CH₂), gives rise to compounds of Formula I-155. Treatment of sulfenamides I-155 with iodosobenzene in the presence of alcohols R₆OH gives rise to the sulfonimidates of Formula I-157 (see D. Leca et al, Organic Letters (2002) 4:4093). Alternatively, compounds I-155 (Z=—CH═CH) may be optionally reduced to the saturated analogs I-156 (Z=CH₂—CH₂—), which are converted to the corresponding sulfonimidates I-157.

Treatment of readily available sulfonylchlorides 154.1 with amines HN(R₄)₂ and base gives rise to compounds of Formula I-154.2.

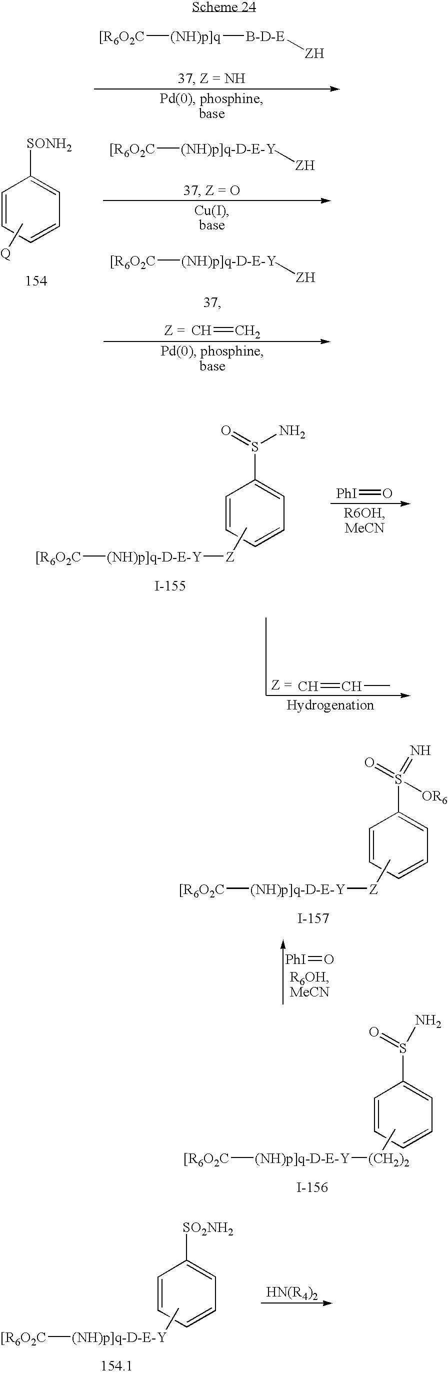

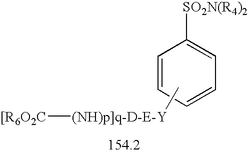

Compounds of Formula I wherein Q is taken from Q-33 are prepared as shown in Scheme 25. Readily available nitriles 158 are reacted with amines 37 (Z=NH), alcohols 37 (Z=O), or alkenes 37 (Z=—CH═CH₂) to afford compounds of Formula I-159. Compounds I-159 (wherein Z=CH═CH—) are optionally reduced to their saturated analogs I-160 by standard catalytic hydrogenation conditions. Treatment of compounds I-159 or I-160 with a metal azide (preferably sodium azide or zinc azide) gives rise to tetrazoles of Formula I-161.

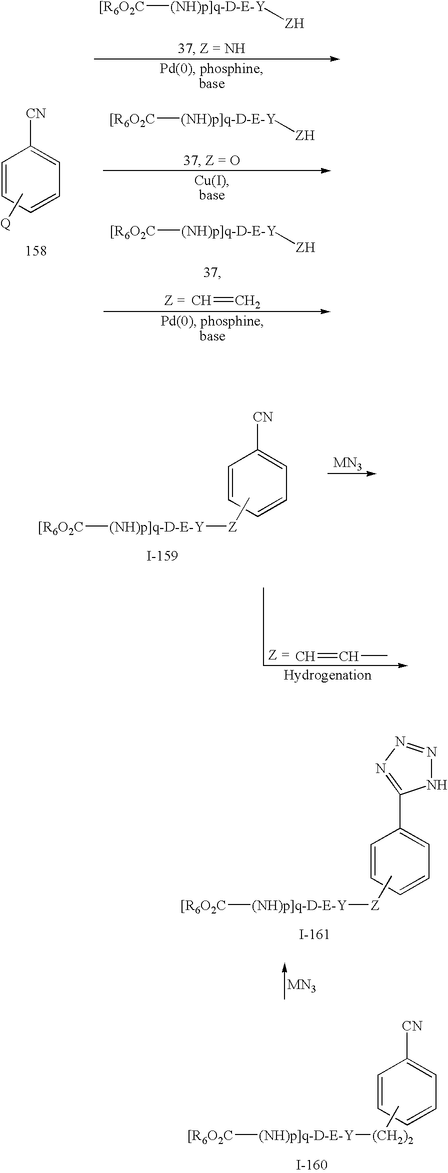

Compounds of Formula I wherein Q is taken from Q-34 are prepared as shown in Scheme 26. Readily available esters 162 are reacted with amines 37 (Z=NH), alcohols 37 (Z=0), or alkenes 37 (Z=—CH═CH₂) to afford compounds of Formula I-163. Compounds I-163 (wherein Z is —CH═CH—) are optionally converted to the saturated analogs I-164 by standard hydrogenation conditions. Compounds I-163 or I-164 are converted to the desired phosphonates I-165 by an Arbuzov reaction sequence involving reduction of the esters to benzylic alcohols, conversion of the alcohols to the benzylic bromides, and treatment of the bromides with a tri-alkylphosphite. Optionally, phosphonates I-165 are converted to the flourinated analogs I-166 by treatment with diethylaminosulfur trifluoride (DAST).

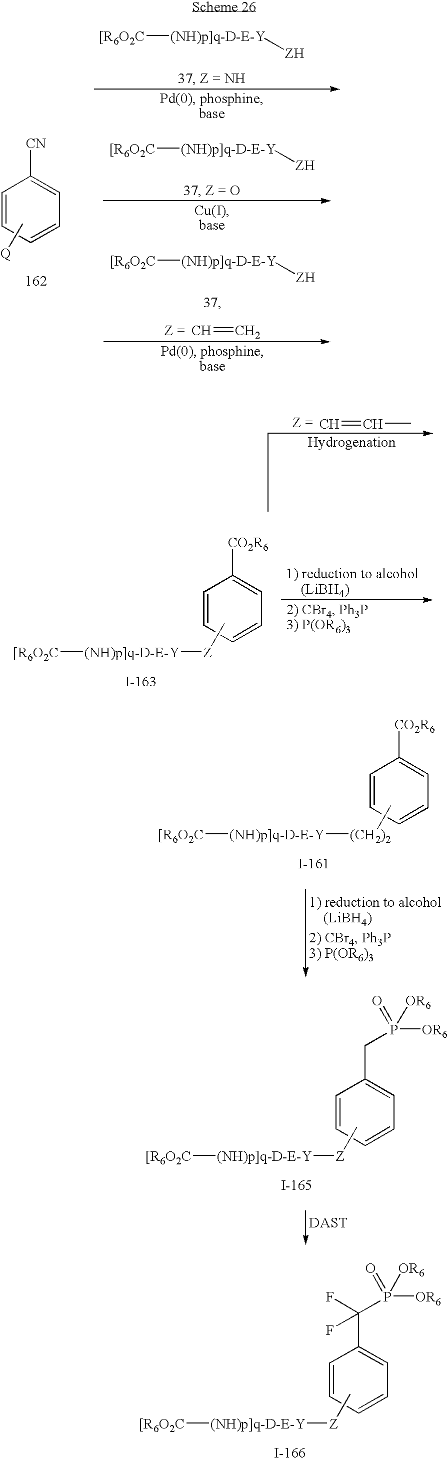

Compounds of Formula I wherein Q is taken from Q-34 are also prepared as illustrated in Scheme 27.1. Intermediate 8a, wherein M is a suitable leaving group such as chloride, bromide or iodide, is refluxed with triethyl phosphite and the resulting phosphoryl intermediate saponified under mild conditions to yield I-165.

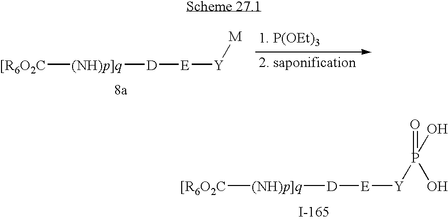

Compounds of Formula I wherein Q is taken from Q-35 are prepared according to Scheme 27. Readily available acid chlorides 167 are reacted with oxazolidones in the presence of base to afford the N-acyl oxazolidinones 168. Intermediate 168 are reacted with amines 37 (Z=NH), alcohols 37 (Z=O), or alkenes 37 (Z=—CH═CH₂) to afford the N-acyl oxazolidinones of Formula I-169. Compounds I-169 (wherein Z is —CH═CH—) are optionally converted to the saturated analogs I-170 under standard hydrogenation conditions.

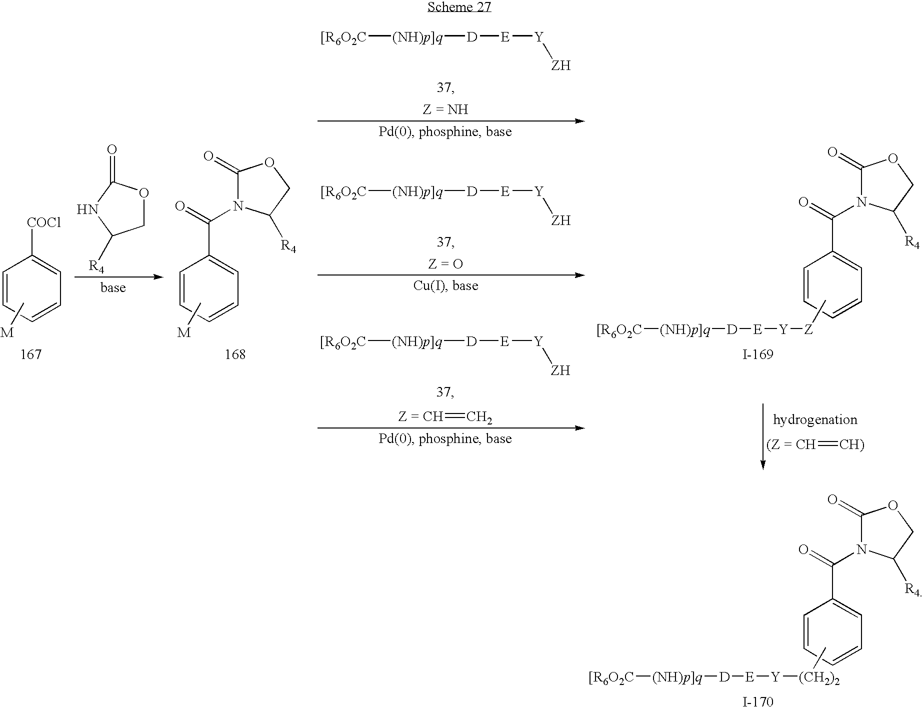

Compounds of Formula I wherein Q is taken from Q-35A are prepared as illustrated in Schemes 28.1 and 28.2. Reductive alkylation of the t-butylsulfide substituted piperazines with the readily available aldehydes 131 gives rise to the benzylic piperazines 171. Intermediates 171 are reacted with amines 37 (Z=NH), alcohols 37 (Z=0), or alkenes 37 (Z=—CH═CH₂) to give compounds 172, 173, or 174, respectively. Optionally, intermediates 174 are converted to the saturated analogs 175 under standard hydrogenation conditions.

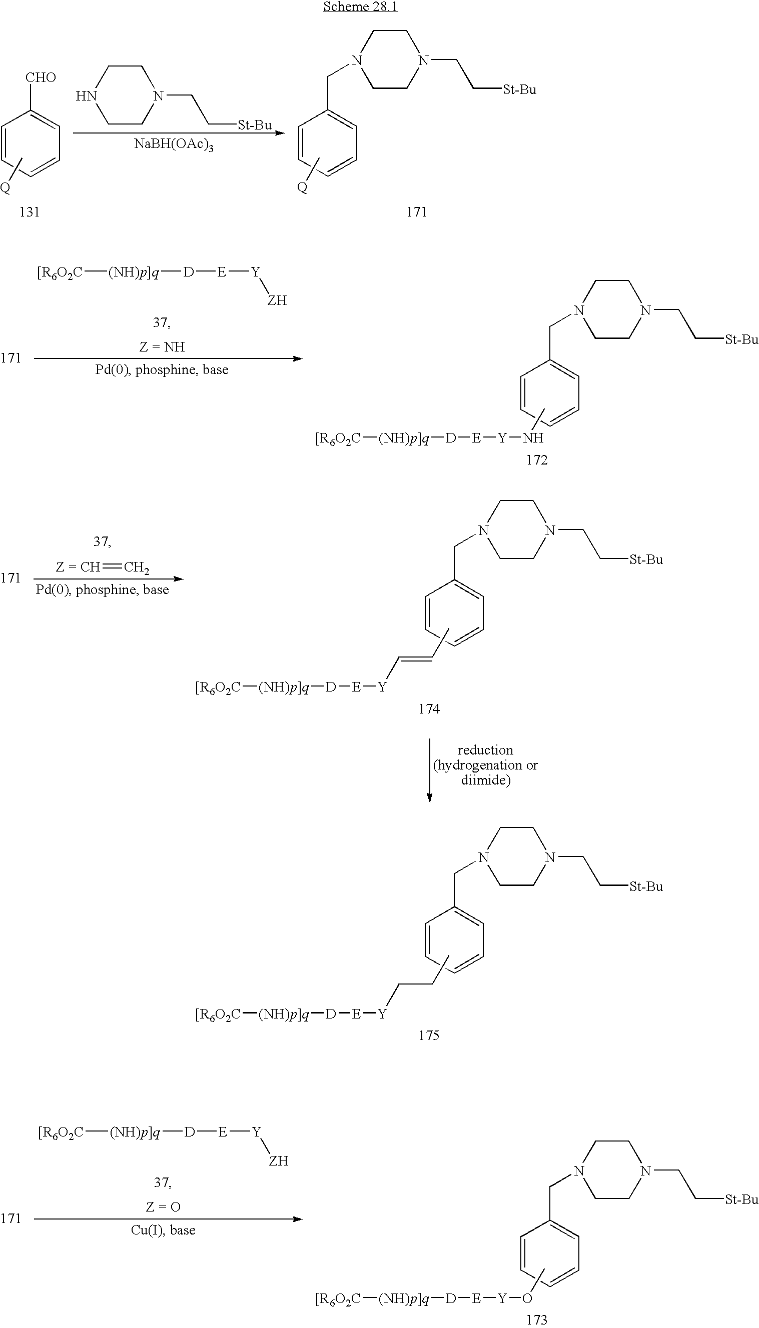

Scheme 28.2 illustrates the conversion of intermediate t-butylsulfides 172-175 to the sulfonic acids, employing a two step process involving acid-catalyzed deprotection of the t-butyl sulfide to the corresponding mercaptans, and subsequent peracid oxidation (preferably with peracetic acid or trifluoroperacetic acid) of the mercaptans to the desired sulfonic acids of Formula I-176.

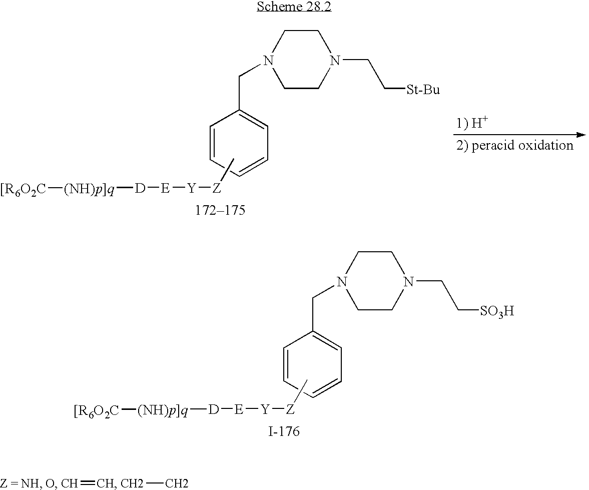

In some instances a hybrid p38-alpha kinase inhibitor is prepared which also contains an ATP-pocket binding moiety or an allosteric pocket binding moiety R₁—X-A. The synthesis of functionalized intermediates of formula R₁—X-A are accomplished as shown in Scheme 29. Readily available intermediates 177, which contain a group M capable of oxidative addition to palladium(0), are reacted with amines 178 (X═NH) under Buchwald Pd(0) amination conditions to afford 179. Alternatively amines or alcohols 178 (X═NH or O) are reacted thermally with 177 in the presence of base under nuclear aromatic substitution reaction conditions to afford 179. Alternatively, alcohols 178 (X═O) are reacted with with 177 under Buchwald copper(I)-catalyzed conditions to afford 179. In cases where p=1, the carbamate of 179 is removed, preferably under acidic conditions when R₆ is t-butyl, to afford amines 180. In cases where p=0, the esters 179 are converted to the acids 181 preferably under acidic conditions when R₆ is t-butyl.

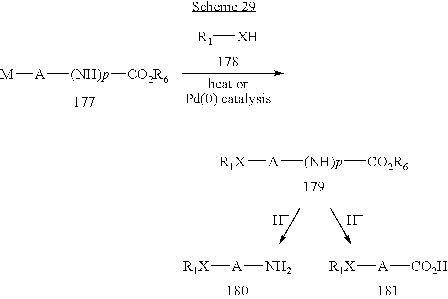

Another sequence for preparing amines 180 is illustrated in Scheme 30. Reaction of amines or alcohols 178 with nitro(hetero)arenes 182 wherein M is a leaving group, preferably M is fluoride, or M is a group capable of oxidative insertion into palladium(0), preferably M is bromo, chloro, or iodo, gives intermediates 183. Reduction of the nitro group under standard hydrogenation conditions or treatment with a reducing metal, such as stannous chloride, gives amines 180.

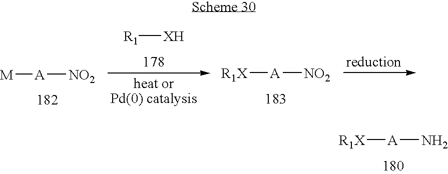

In instances when hybrid p38-alpha kinase inhibitors are prepared, compounds of Formula I-184 wherein q is 1 may be converted to amines I-185 (p=1) or acids I-186 (p=0) by analogy to the conditions described in Scheme 29. Compounds of Formula I-184 are prepared as illustrated in previous schemes 1.1, 2.1, 2.2, 3, 4, 5, 6, 7.1, 7.2, 8, 9, 10, 12, 14, 16.2, 17.2, 18, 19.1, 19.2, 19.3, 20, 21, 22, 23, 24, 25, 26, 27, or 28.2.

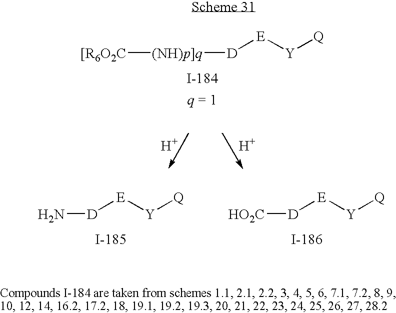

The preparation of inhibitors of Formula I which contain an amide linkage CO—NH-connecting the oxyanion pocket binding moieties and R₁—X-A moieties are shown in Scheme 32. Treatment of acids 181 with an activating agent, preferably PyBOP in the presence of di-iso-propylethylamine, and amines I-185 gives compounds of Formula I. Alternatively, retroamides of Formula I are formed by treatment of acids I-186 with PyBOP in the presence of di-iso-propylethylamine and amines 180.

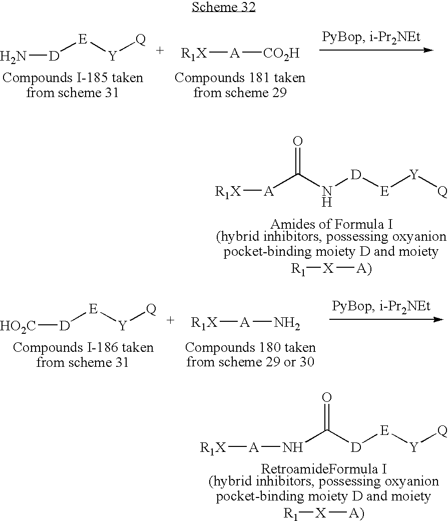

The preparation of inhibitors of Formula I which contain an urea linkage NH—CO—NH— connecting the oxyanion pocket binding moieties and the R₁—X-A moieties are shown in Scheme 33. Treatment of amines I-185 with p-nitrophenyl chloroformate and base affords carbamates 187. Reaction of 187 with amines 180 gives ureas of Formula I.

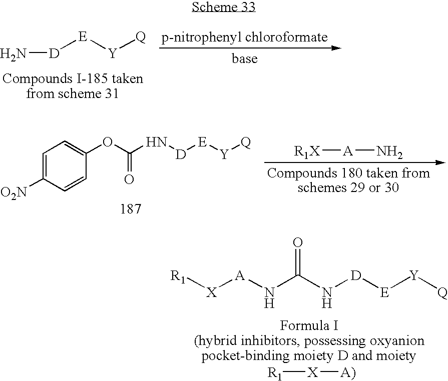

Alternatively, inhibitors of Formula I which contain an urea linkage NH—CO—NH-connecting the oxyanion pocket binding moieties and the R₁—X-A moieties are prepared as shown in Scheme 33. Treatment of amines 180 with p-nitrophenyl chloroformate and base affords carbamates 188. Reaction of 188 with amines I-185 gives ureas of Formula I.

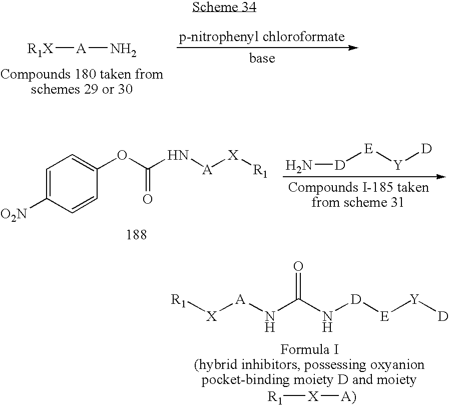

The preparation of inhibitors of Formula I.B can be generally accomplished starting from a variety of readily available beta-ketonitriles 189, wherein R₄₀ is alkyl, phenyl, or perfluoroalkyl. As illustrated in Scheme 35, reaction of 189 with an alcohol R₄OH, preferably methanol or ethanol, under anhydrous acidic conditions, preferably anhydrous HCl, leads to the formation of imidates 190. Reaction of 190 with acyl chlorides, isocyanates, para-nitrophenylcarbamates, or substituted chloroformates in the presence of a base, preferably pyridine, triethylamine, di-iso-propylethylamine, Barton's base, or an alkali metal carbonate, affords key intermediates 191 and 192 as a mixture of tautomers, wherein T is alkylene, NH, O, or when T is absent, then the carbonyl side chain and A are connected by a direct bond.

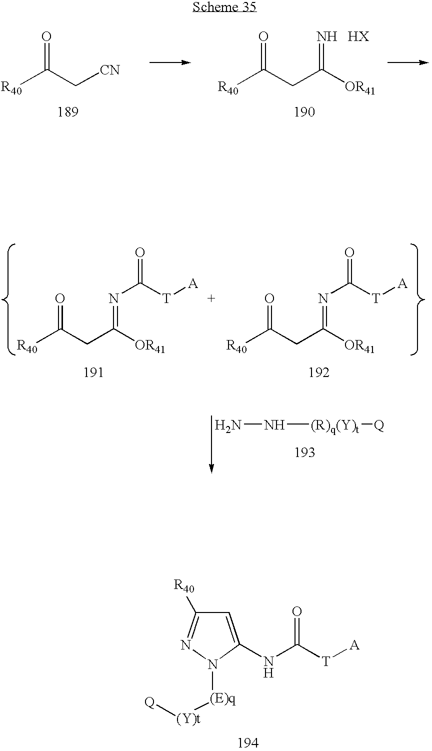

The mixture of tautomers 191/192 are not separated from each other, but are reacted as a mixture with a substituted hydrazine 193, wherein the Q moiety is optionally protected by a protecting group that diminishes its reactivity with the 191/192 mixture. This cyclodehydration reaction is performed in the presence of base, acid catalysis, or under neutral conditions optionally in the presence of a dehydrating agent to afford the desired pyrazoles 194. Preferable reaction solvents include dichloromethane, ethyl acetate, acetonitrile, or an alcoholic solvent taken from methanol, ethanol, or 2-propanol.

The reaction sequence initiating from 190 and yielding 194 may take place as two separate reactions, wherein the tautomeric mixture 191/192 is isolated, and then in a second reaction step this 191/192 mixture is reacted with a substituted hydrazine 193 to afford the desired pyrazoles 194. Alternatively, the reaction sequence initiating from 190 and yielding 194 may take place in a one-pot procedure, without isolation of the intermediate 191/192 mixture.

In a further modification, the reaction sequence initiating from 190 and yielding 194 may take place in a parallel array format, wherein phase-trafficking reagents, including scavenging reagents, are utilized to allow purification and isolation of intermediates and products. Scheme 36 illustrates this modification. Excess imidate 190 is reacted with a limiting amount of electrophile in the presence of a polymer-supported base 195 to afford the acylated imidates 191/192 as a mixture of tautomers. The crude mixture of 191/192 is optionally purified by incubation with a polymer-supported electrophile 196, preferably a polymer-supported isocyanate or acid chloride. Reaction of 196 with any remaining imidate 190 sequesters this imidate as polymer-supported 197. Filtration gives purified 191/192. In the second step, purified acylated imidates 191/192 are reacted with the substituted hydrazines 193 to afford desired crude products 194. A polymer-supported hydrazine 198 is optionally utilized to scavenge any remaining 191/192 from solution phase as derivatized 199. Filtration gives rise to purified desired pyrazoles 194.

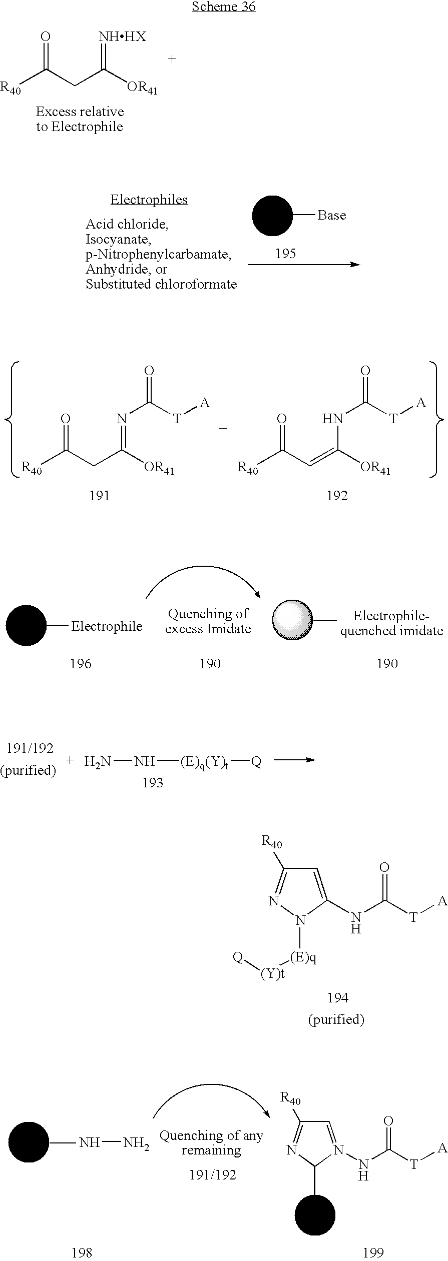

Scheme 37 illustrates the preparation of compounds wherein Q is Q-40. Readily available amine 200, wherein P is a suitable amine-protecting group or a group convertible to an amine group, is reacted with p-nitrophenyl chloroformate to give rise to carbamate 201. Intermediate 201 is reacted with a substituted amino acid ester with a suitable base to afford urea 202. Further treatment with base results in cyclization to afford hydantoin 203. The protecting group P is removed to afford the key amine-containing intermediate 204. Alternatively, if P is a nitro group, then 203 is converted to 204 under reducing conditions such as iron/HCl, tin(II) chloride, or catalytic hydrogenation. Amine 204 is converted to 205A by reaction with an isocyanate; 204 is converted to amide 205B by reaction with an acid chloride, acid anhydride, or a suitable activated carboxylic acid in the presence of a suitable base; 204 is converted to carbamate 205C by reaction with a substituted alkyl or aryl chloroformate in the presence of a suitable base.

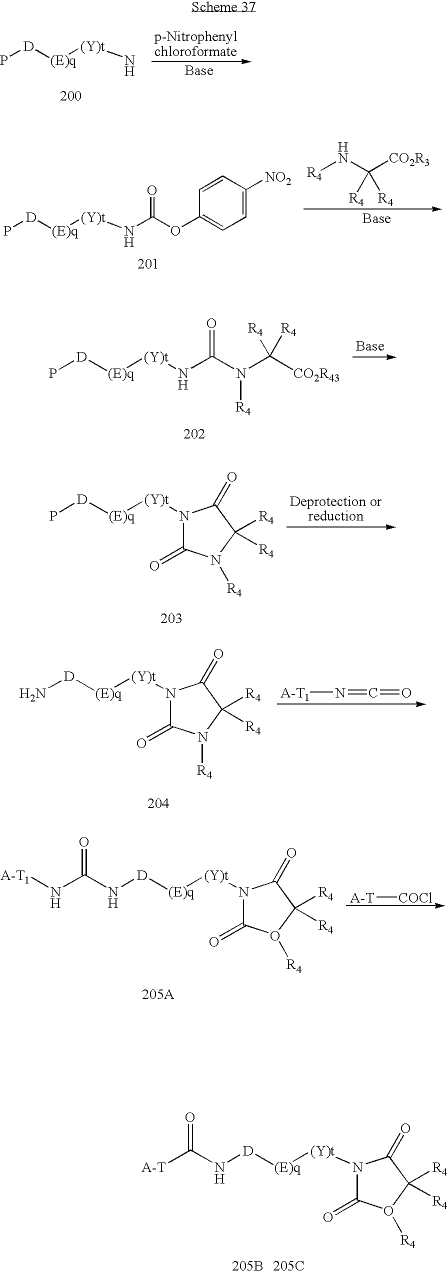

Scheme 38 illustrates the synthesis of key substituted hydrazine 210. This hydrazine can be converted into compounds of formula I.B using the methods previously outlined in Schemes 35 and 36. The nitrophenyl substituted amine 206 is reacted with p-nitrophenyl chloroformate to give rise to carbamate 207. Reaction of 207 with a suitable amino acid ester affords urea 208, which is cyclized under basic conditions to give hydantoin 209. Reduction of the nitro group of 209, diazotization of the resulting amine, and reduction of the diazonium salt affords key hydrazine 210.

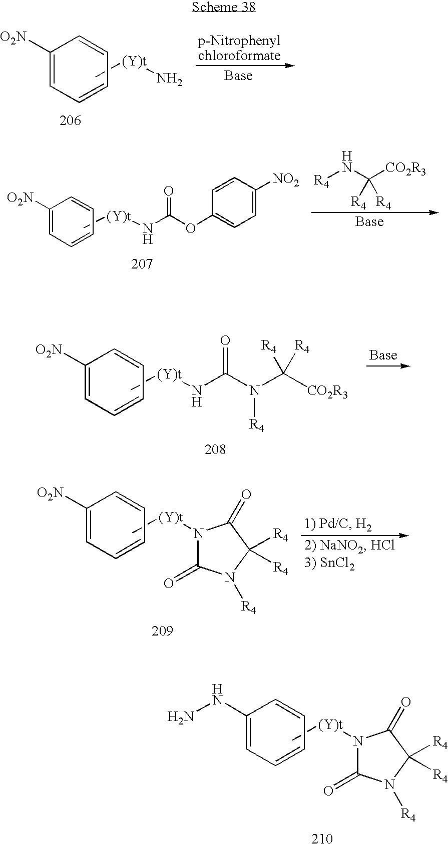

Scheme 39 illustrates the synthesis of key substituted hydrazines 213 and 216, utilized to prepare compounds of formula I.B wherein Q is Q-42 and G is oxygen. Nitrophenol 211 is reacted with an alpha-hydroxy acid, wherein R₄₂ is H or alkyl and R₄₃ is alkyl, under Mitsunobu reaction conditions to give 212; alternatively 211 is reacted under basic conditions with a carboxylic acid ester containing a displaceable Qₓ group to afford 212. Conversion of 212 to the hydrazine 213 is accomplished by standard procedures as described above.

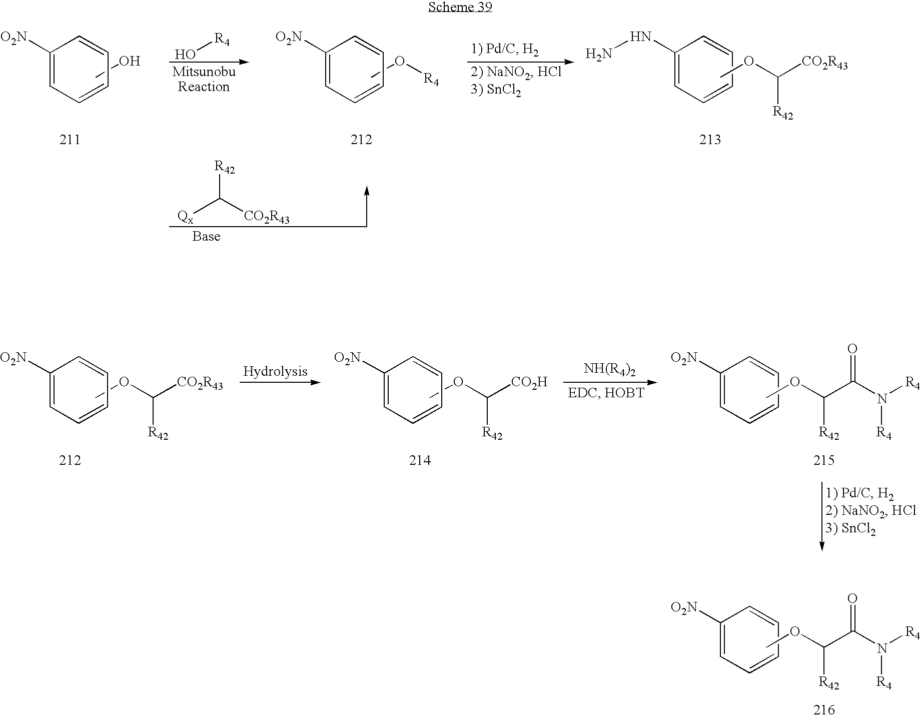

Alternatively, the ester group of 212 is hydrolyzed to afford carboxylic acid 214, which is reacted with an amine NH(R₄)₂ in the presence of a coupling reagent, preferably EDC/HOBT, to give amide 215. Conversion of 215 to the substituted hydrazine 216 is accomplished by standard procedures. Hydrazines 213 and 216 can be converted into compounds of formula I.B using the methods previously outlined in Schemes 35 and 36.

Scheme 40 illustrates the synthesis of key substituted hydrazines 219 and 222, utilized to prepare compounds of formula I.B wherein Q is Q-42 and G is methylene. Nitrophenyl bromide 217 is reacted with an alpha-beta unsaturated ester using Pd(0) catalyzed Heck reaction conditions, to afford ester 218. This intermediate is converted to the substituted hydrazine 219 by standard procedures involving concomitant reduction of the alpha-beta unsaturated bond. Alternatively, ester 218 is hydrolyzed to the carboxylic acid 220, which is reacted with an amine NH(R₄)₂ in the presence of a coupling reagent, preferably EDC/HOBT, to give amide 221. Conversion of 221 to the substituted hydrazine 222 is accomplished by standard procedures. Hydrazines 219 and 222 can be converted into compounds of formula I.B using the methods previously outlined in Schemes 35 and 36.

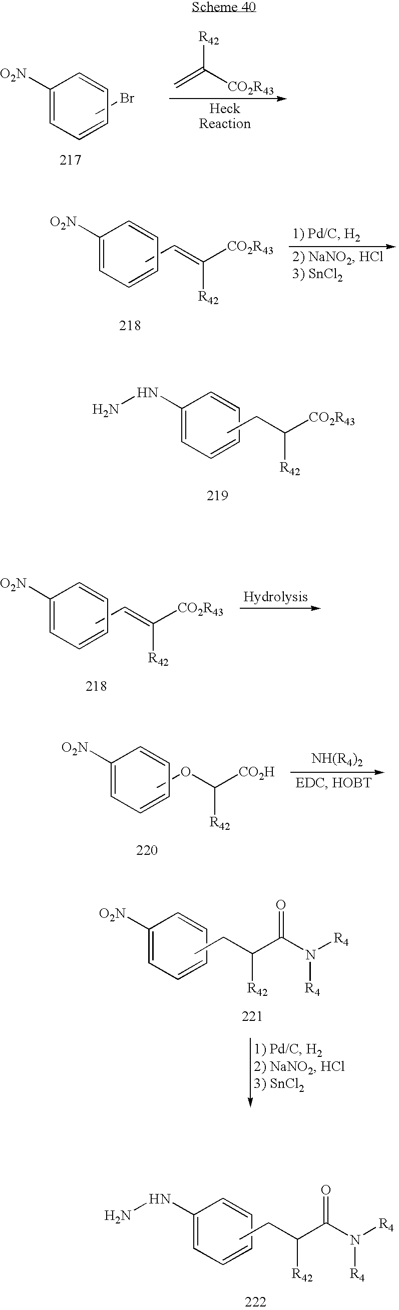

Scheme 41 illustrates an alternative synthesis of key substituted hydrazines 225 and 228, utilized to prepare compounds of formula I.B wherein Q is Q-42, G is methylene, and one or both of R₄₂ are carbon-containing substituents. Nitrobenzyl acetate 223 is reacted with a substituted silylketene acetal to afford ester 224. This intermediate is converted to the substituted hydrazine 225 by standard procedures. Alternatively, ester 223 is hydrolyzed to the carboxylic acid 226, which is reacted with an amine NH(R₄)₂ in the presence of a coupling reagent, preferably EDC/HOBT, to give amide 227. Conversion of 227 to the substituted hydrazine 228 is accomplished by standard procedures. Hydrazines 225 and 228 can be converted into compounds of formula I.B using the methods previously outlined in Schemes 35 and 36.

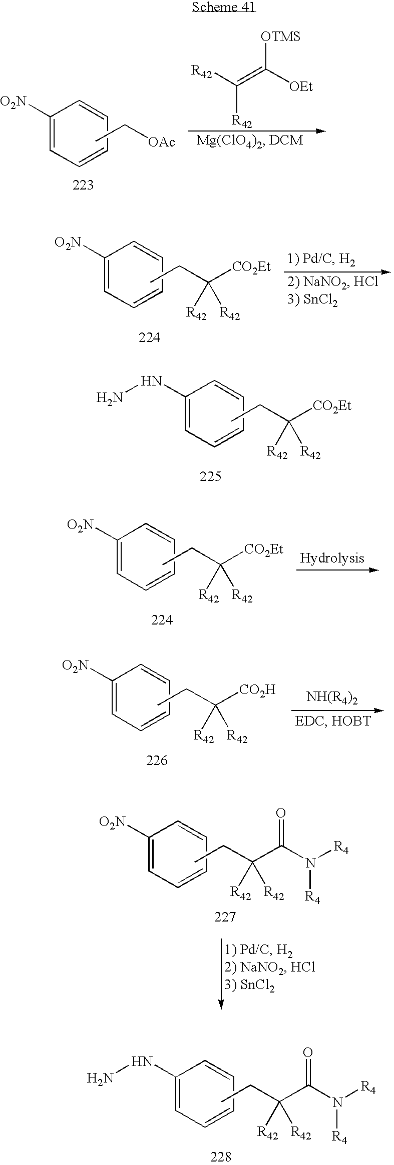

Scheme 42 illustrates an alternative synthesis of key substituted hydrazines 231 and 234, utilized to prepare compounds of formula I.B wherein Q is Q-42 and G is NH. Iodoaniline 229 is reacted with an alpha-keto ester under reductive amination conditions, preferably sodium triacetoxyborohydride, to afford ester 230. This intermediate is converted to the substituted hydrazine 231 by Cu(I)-catalyzed reaction with N—BOC hydrazine. Alternatively, ester 231 is hydrolyzed to the carboxylic acid 232, which is reacted with an amine NH(R₄)₂ in the presence of a coupling reagent, preferably EDC/HOBT, to give amide 233. Conversion of 233 to the substituted hydrazine 234 is accomplished by Cu(I)-catalyzed reaction with N—BOC hydrazine. Hydrazines 231 and 234 can be converted into compounds of formula I.B using the methods previously outlined in Schemes 35 and 36, after acid-catalyzed removal of the hydrazine N—BOC protecting group, preferably with trifluoroacetic acid or HCl-dioxane.

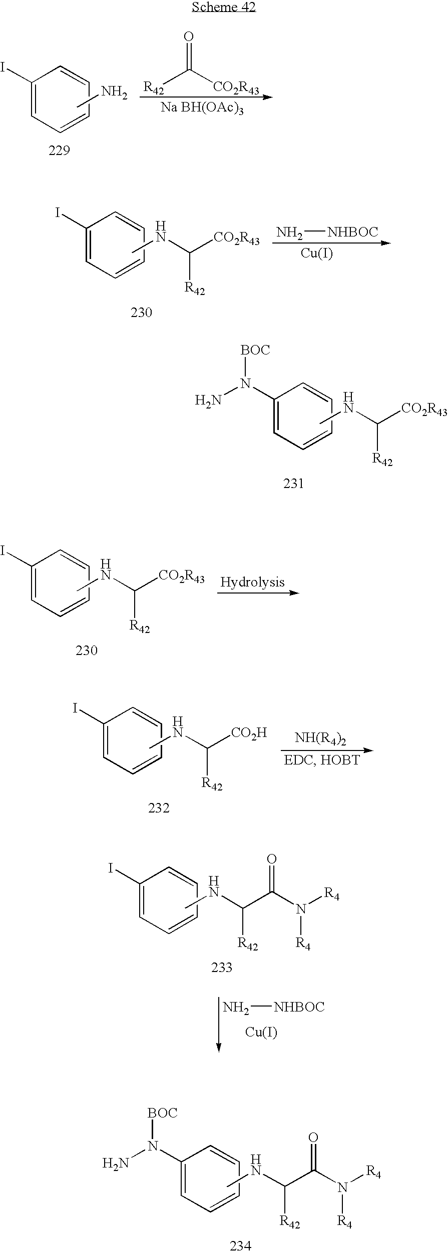

Scheme 43 illustrates an alternative synthesis of key substituted hydrazine 239 utilized to prepare compounds of formula I.B wherein Q is Q-42, G is oxygen, and X is taken from piperidinyl, piperazinyl, thiomorphorlino sulfone, or 4-hydroxypiperinyl. Iodophenol 235 is reacted with an alpha-hydroxy acid under Mitsunobu reaction conditions to give 236; alternatively 235 is reacted under basic conditions with a carboxylic acid ester containing a displaceable Q group to afford 236. Ester 236 is hydrolyzed to the carboxylic acid 237 which is reacted with an amine X—H in the presence of a coupling reagent, preferably EDC/HOBT, to give amide 238. Conversion of 238 to the substituted hydrazine 239 is accomplished by Cu(I)-catalyzed reaction with N—BOC hydrazine. Hydrazine 239 can be converted into compounds of formula I.B using the methods previously outlined in Schemes 35 and 36, after acid-catalyzed removal of the hydrazine N—BOC protecting group, preferably with trifluoroacetic acid or HCl-dioxane.

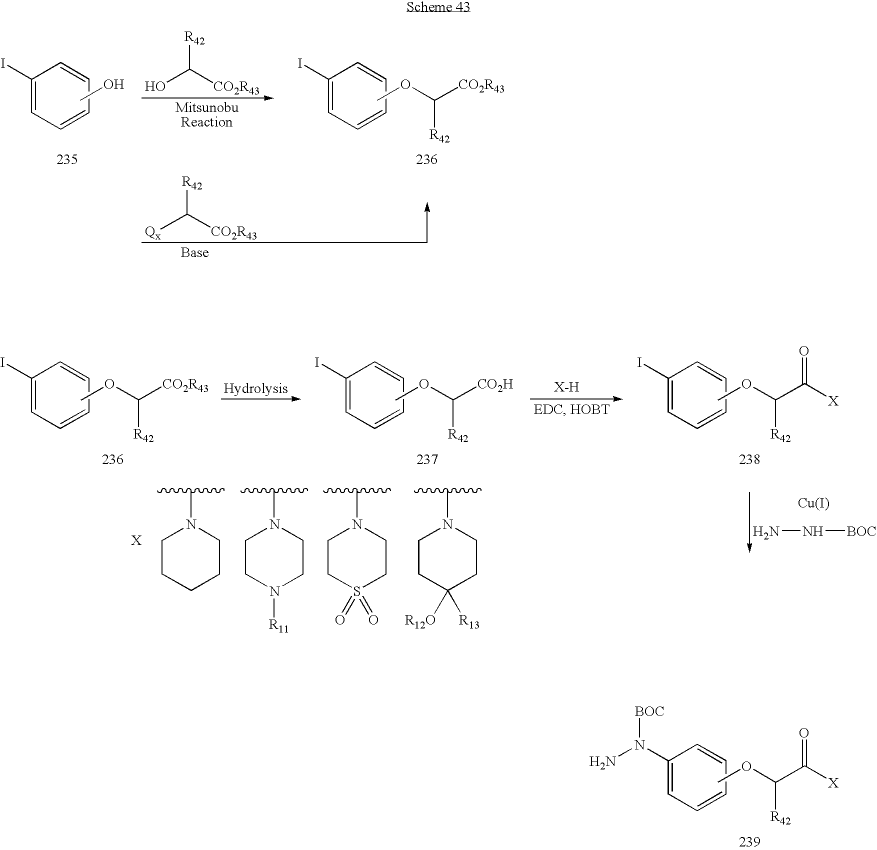

Scheme 44 illustrates an alternative synthesis of key substituted hydrazine 241, utilized to prepare compounds of formula I.B wherein Q is Q-42, G is NH, and X is taken from piperidinyl, piperazinyl, thiomorphorlino sulfone, or 4-hydroxypiperinyl. Carboxylic acid 237 is reacted with an amine X—H in the presence of a coupling reagent, preferably EDC/HOBT, to give amide 240. Conversion of 240 to the substituted hydrazine 241 is accomplished by Cu(I)-catalyzed reaction with N—BOC hydrazine. Hydrazine 241 can be converted into compounds of formula I.B using the methods previously outlined in Schemes 35 and 36, after acid-catalyzed removal of the hydrazine N—BOC protecting group, preferably with trifluoroacetic acid or HCl-dioxane.

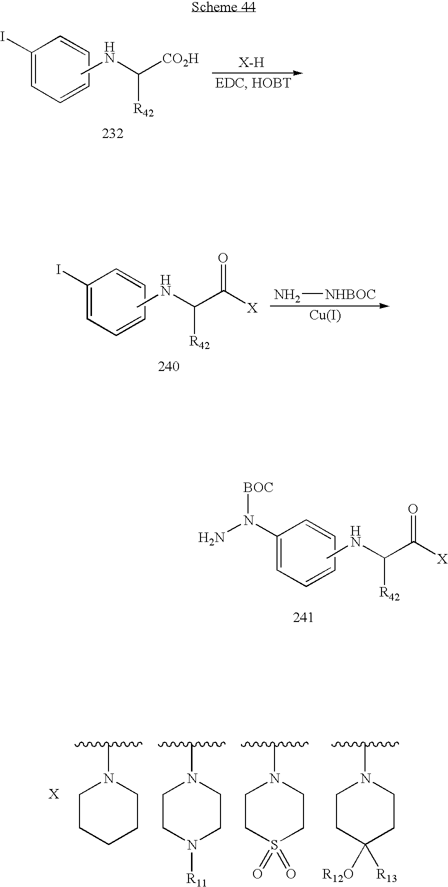

Scheme 45 illustrates an alternative synthesis of key substituted hydrazine 246, utilized to prepare compounds of formula I.B wherein Q is Q-42, G is methylene, and X is taken from piperidinyl, piperazinyl, thiomorphorlino sulfone, or 4-hydroxypiperinyl. Iodobenzyl acetate 242 is reacted with a substituted silylketene acetal to afford ester 243. Ester 243 is hydrolyzed to the carboxylic acid 244, which is reacted with an amine X—H in the presence of a coupling reagent, preferably EDC/HOBT, to give amide 245. Conversion of 245 to the substituted hydrazine 246 is accomplished by Cu(I)-catalyzed reaction with N—BOC hydrazine. Hydrazine 246 can be converted into compounds of formula I.B using the methods previously outlined in Schemes 35 and 36, after acid-catalyzed removal of the hydrazine N—BOC protecting group, preferably with trifluoroacetic acid or HCl-dioxane.

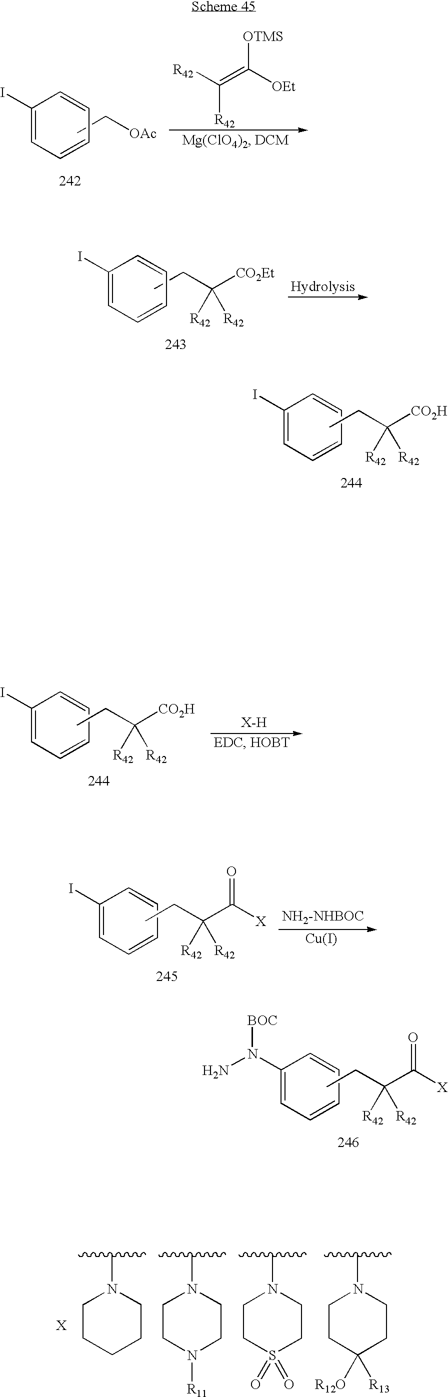

Scheme 46 illustrates an alternative synthesis of key substituted hydrazines 248, 252, and 255, utilized to prepare compounds of formula I.B wherein Q is Q-47 or Q-48. Nitrophenol 211 is reacted with a substituted alcohol under Mitsunobu reaction conditions to afford 247; alternatively 211 is alkylated with R₄-Qₓ, wherein Qₓ is a suitable leaving group, under basic reaction conditions, to give rise to 247. Conversion of 247 to the substituted hydrazine 248 is accomplished under standard conditions.

The nitrobenzoic acid 249 is converted to the acid fluoride 250 by reaction with a fluorinating reagent, preferably trifluorotriazine. Treatment of acid fluoride 250 with a nucleophilic fluoride source, preferably cesium fluoride and tetra-n-butylammonium fluoride, affords the alpha-alpha-difluorosubstituted carbinol 251. Conversion of 251 to the substituted hydrazine 252 is accomplished under standard conditions.

Nitrobenzaldehyde 253 is reacted with trimethylsilyltrifluoromethane (TMS-CF₃) and tetra-n-butylammonium fluoride to give rise to trifluoromethyl-substituted carbinol 254. Conversion of 254 to the substituted hydrazine 255 is accomplished under standard conditions. Hydrazines 248, 252, and 255 can be converted into compounds of formula I.B using the methods previously outlined in Schemes 35 and 36.

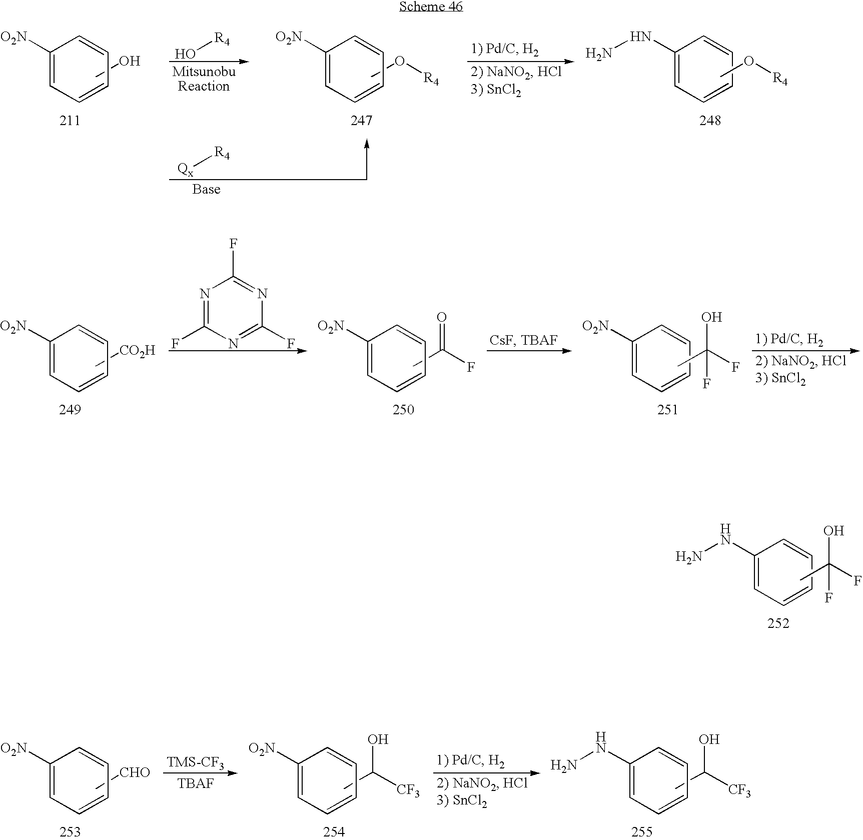

Scheme 47 illustrates the preparation of compounds of formula I.B wherein Q is Q-59. p-Nitrophenylcarbamate 201 is reacted with a substituted alpha-hydroxy ester with a suitable base to afford carbamate 256. Further treatment with base results in cyclization to afford oxazolidinedione 257. The protecting group P is removed to afford the key amine-containing intermediate 258; alternatively, if P is a nitro group, then 257 is converted to 258 under reducing conditions such as iron/HCl, tin(II) chloride, or catalytic hydrogenation. Amine 258 is converted to 259A by reaction with an isocyanate wherein T1 is alkylene or a direct bond connecting A and the carbonyl moiety; 258 is converted to amide 259B by reaction with an acid chloride, acid anhydride, or a suitable activated carboxylic acid in the presence of a suitable base; 258 is converted to carbamate 259C by reaction with a substituted alkyl or aryl chloroformate in the presence of a suitable base.

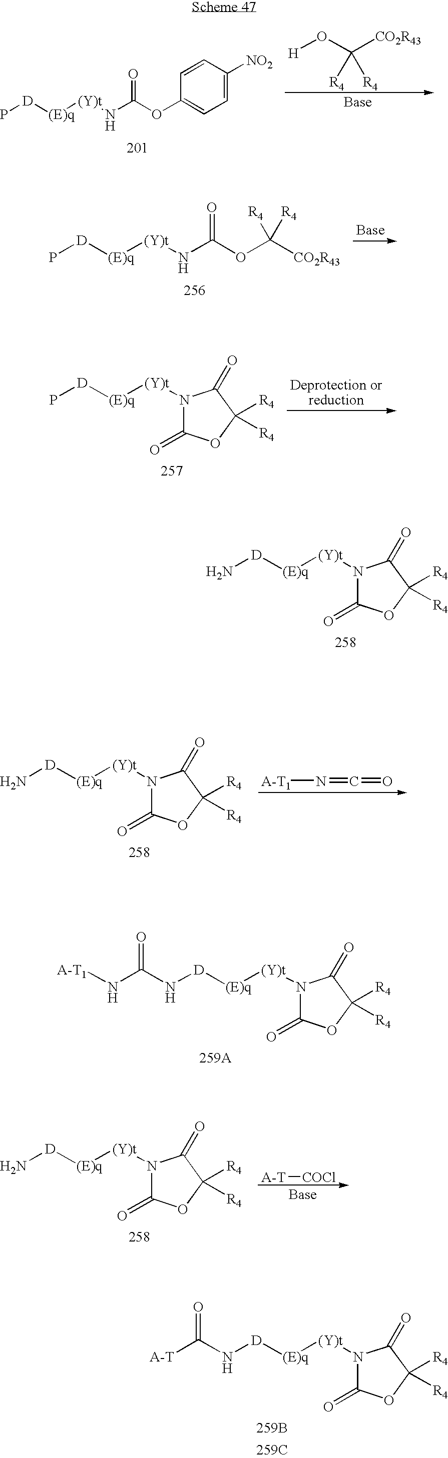

Scheme 48 illustrates an alternative approach to the preparation of compounds of formula I.B wherein Q is Q-59. Amine 260 is reacted with p-nitrophenylchloroformate under basic conditions to give rise to carbamate 261. This intermediate is reacted with an alpha-hydroxy ester in the presence of base to afford carbamate 262. Further treatment with base converts 262 into the oxazolidinedione 263. Conversion of 263 to the substituted hydrazine 264 is accomplished by standard procedures. Hydrazine 264 can be converted into compounds of formula I.B using the methods previously outlined in Schemes 35 and 36.

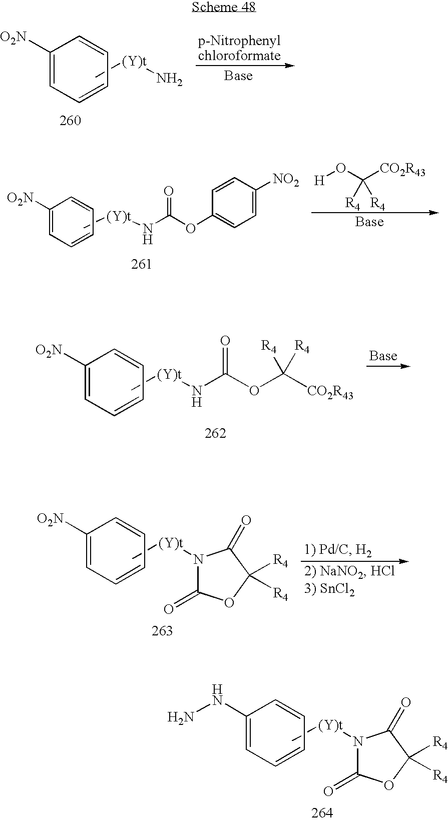

Scheme 49 illustrates the approach to the preparation of compounds of formula I.B wherein Q is Q-57. Amine 265 is reacted with p-methoxybenzylisocyanate under standard conditions to give rise to urea 266. This intermediate is reacted with an oxalyl chloride in the presence of base to afford trione 267. Conversion of 267 to the substituted hydrazine 268 and removal of the p-methoxybenzyl protecting group is accomplished by standard procedures. Hydrazine 264 can be converted into compounds of formula I.B using the methods previously outlined in Schemes 35 and 36.

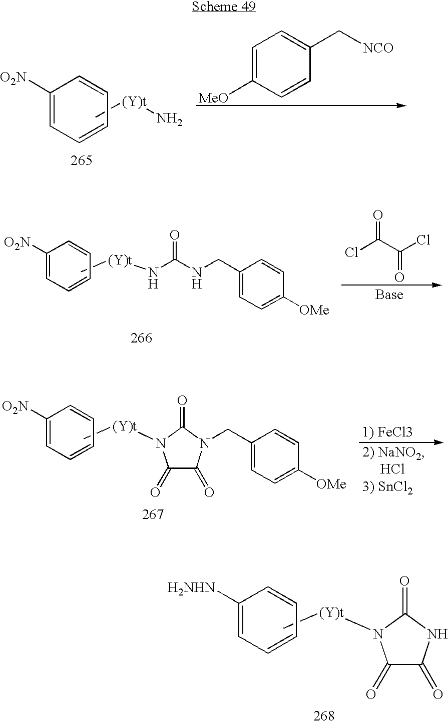

Scheme 50 illustrates an approach to the preparation of compounds of formula I.B wherein Q is Q-56. Amine 269 is reacted with p-methoxybenzylsulfonylchloride under standard conditions to give rise to sulfonylurea 270. This intermediate is reacted with an oxalyl chloride in the presence of base to afford the cyclic sulfonyl urea 271. Conversion of 271 to the substituted hydrazine 272 and removal of the p-methoxybenzyl protecting group is accomplished by standard procedures. Hydrazine 272 can be converted into compounds of formula I.B using the methods previously outlined in Schemes 35 and 36.

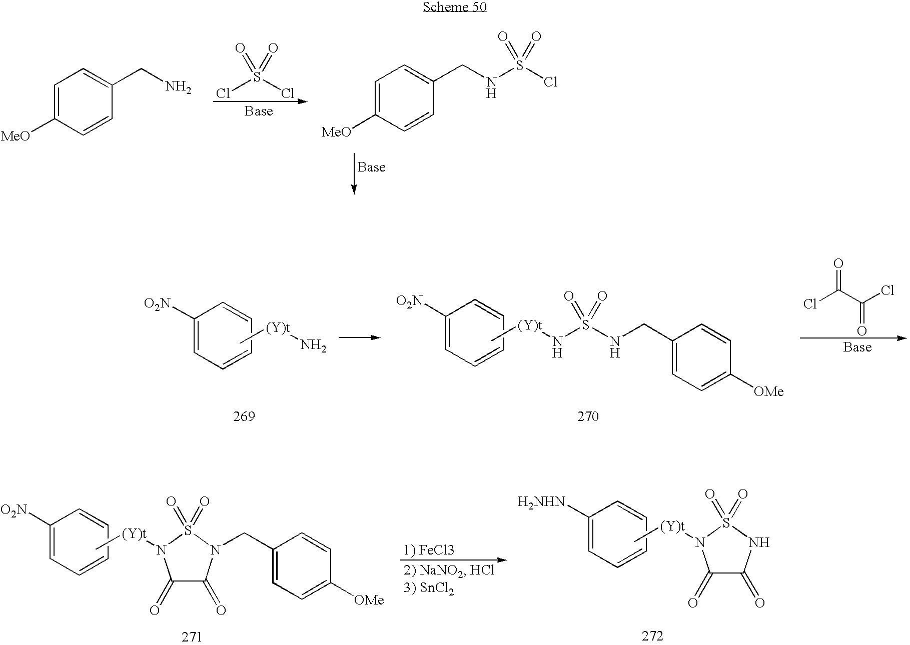

Scheme 51 illustrates an approach to the preparation of compounds of formula I.B wherein Q is Q-58. Amine 273 is reacted with a cyclic anhydride e.g. succinic anhydride in the presence of base under standard conditions to give rise to imide 274. Conversion of 274 to the substituted hydrazine 275 is accomplished by standard procedures. Hydrazine 275, can be converted into compounds of formula I.B using the methods previously outlined in Schemes 35 and 36.

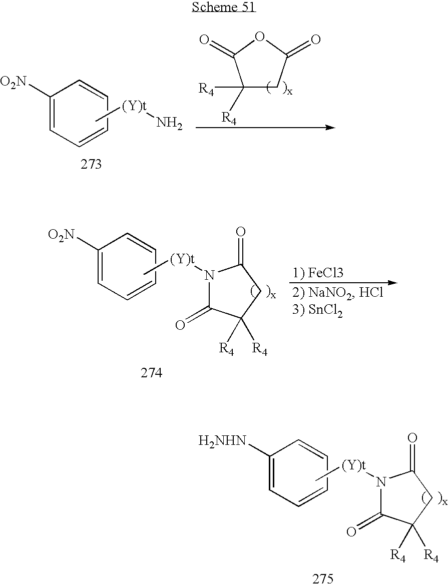

Scheme 52 illustrates an approach to the preparation of compounds of formula I.B wherein Q is Q-54 or Q-55. Carboxylic acid 276 is converted to protected amine 279 under standard conditions, which can be subsequently converted to hydrazine 280 by standard procedures. Hydrazine 280 can be converted into compounds of formula I.B using the methods previously outlined in Schemes 35 and 36 to yield protected amine 283 which is readily deprotected to yield amine 284. Reaction of amine 284 with CDI and amine (R₄)₂NH yields 285 (Q=Q-54). Reaction of amine 284 with the indicated sulfamoylchloride derivative yields 286 (Q=Q-55).

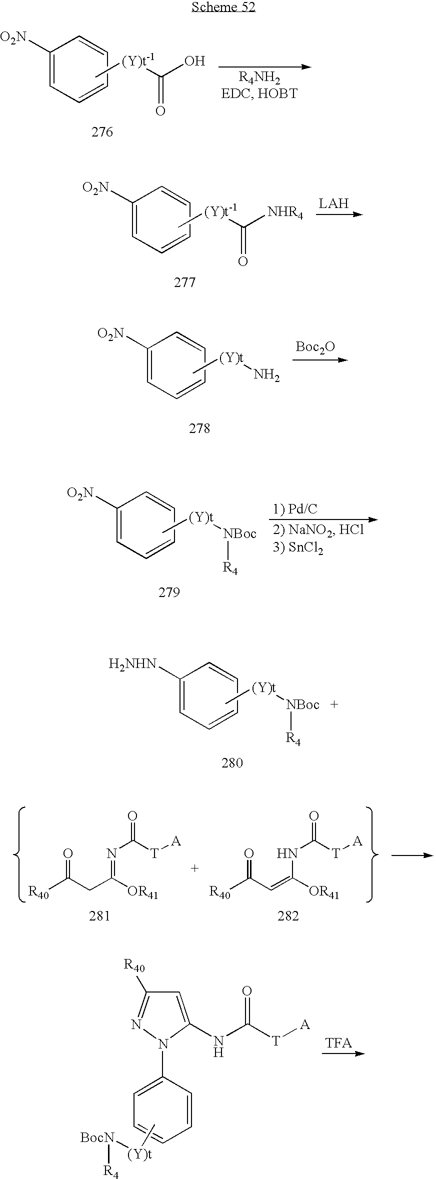

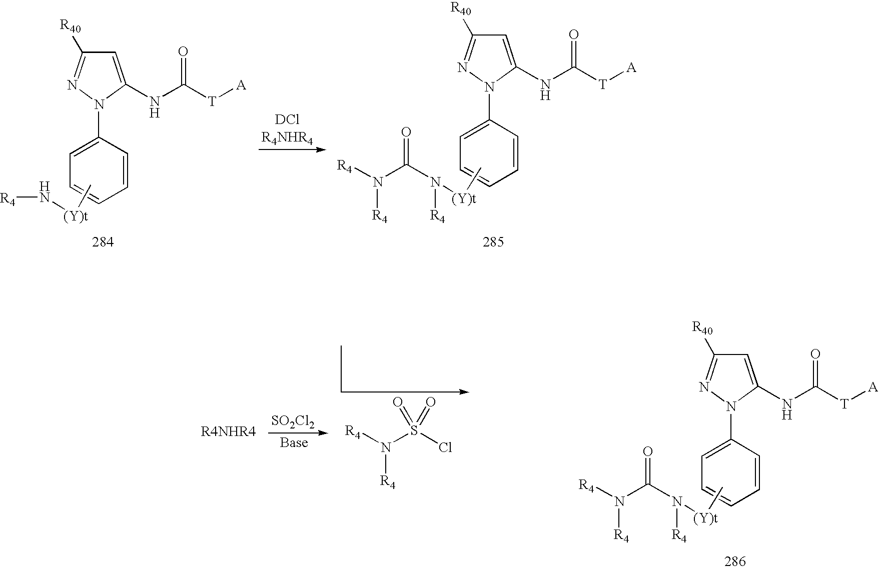

Scheme 53 illustrates an approach to the preparation of compounds of formula I.B wherein Q is Q-49, Q-50 or Q-51. Protected amine 287 (available by several literature procedures) is converted to deprotected hydrazine 288 is accomplished by standard procedures. Hydrazine 288 (Q=Q-49) can be converted into compounds of formula I.B using the methods previously outlined in Schemes 35 and 36. Amine 287 can be deprotected by TFA to yield amine 289 which can be subsequently converted amide 290. Amide 290 is converted to hydrazine 291 (Q=Q-50) by standard procedures, which can be subsequently converted into compounds of formula I.B using the methods previously outlined in Schemes 35 and 36. Alternatively, amine 289 can be reacted with CDI and amine (R₄)₂NH to yield urea 292 (Q=Q-51). Urea 292 is converted to hydrazine 293 (Q=Q-51) by standard procedures, which can be subsequently converted into compounds of formula I.B using the methods previously outlined in Schemes 35 and 36.

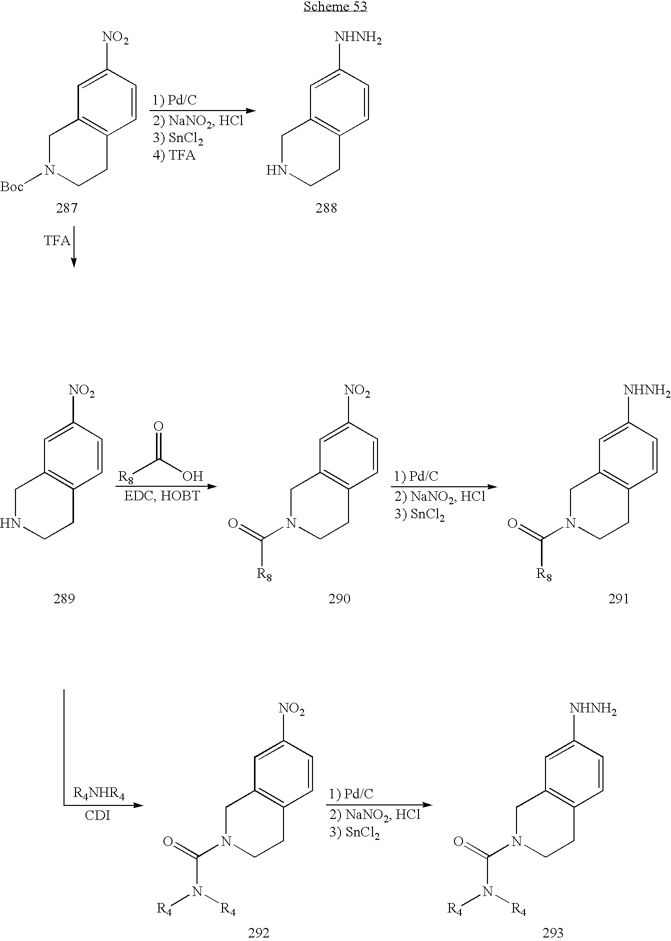

Scheme 54 illustrates an approach to the preparation of compounds of formula I.B wherein Q is Q-52 and Q-53. Protected amine 294 (available by several literature procedures) is converted to protected hydrazine 295 is accomplished by standard procedures. Hydrazine 295 (Q=Q-49) can be converted into compounds of formula I.B to yield protected amine 298 which is readily deprotected to yield amine 299. Reaction of amine 299 with chlorosulfonylisocyanate followed by amine (R₄)₂NH yields 300 (Q=Q-52). Alternatively, reaction of chlorosulfonylisocyanate and amine (R₄)₂NH followed by amine 299 yields 301 (Q=Q-53).

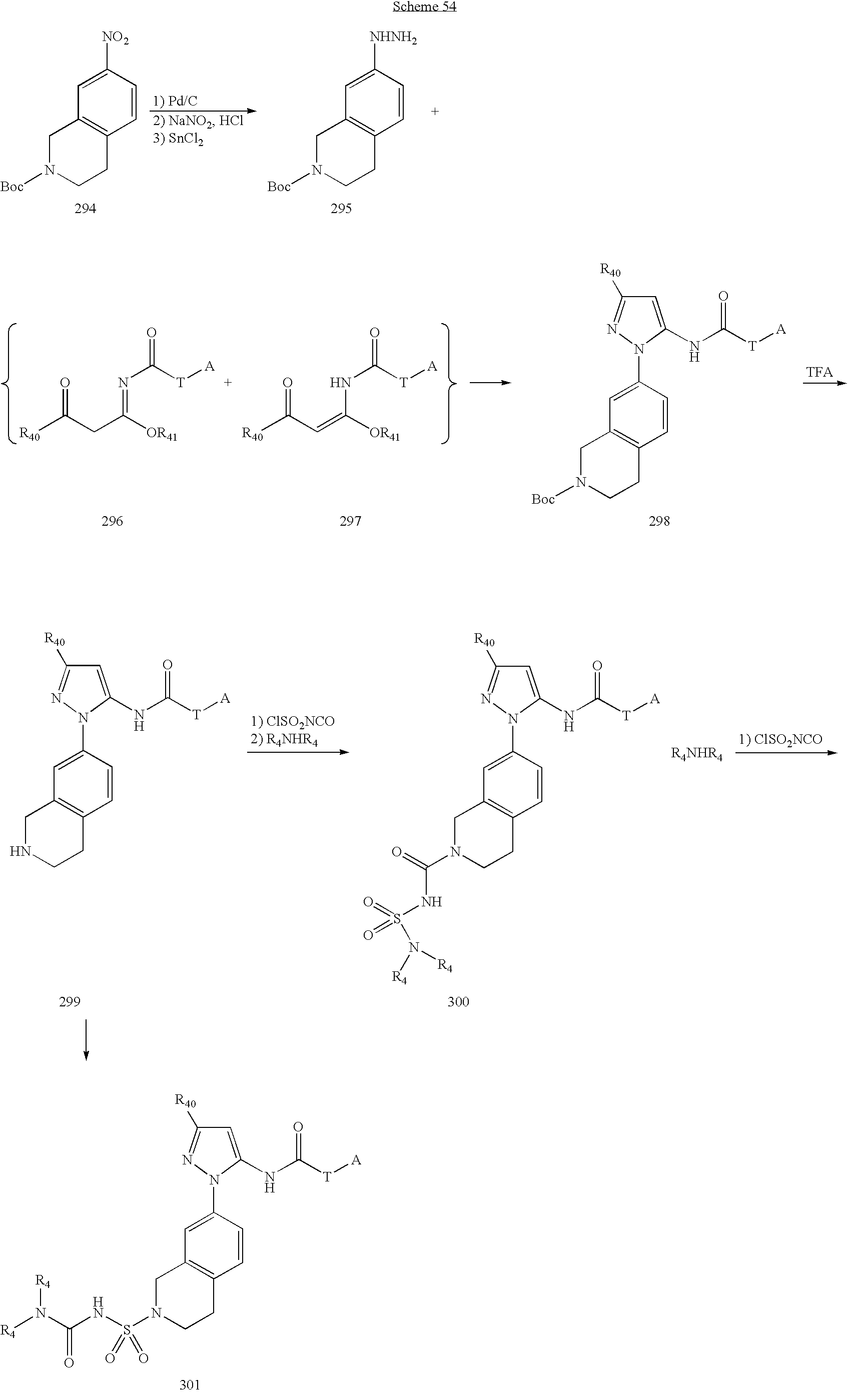

Scheme 55 illustrates an approach to the preparation of compounds of formula I.B wherein Q is Q-36. Amine 302 is reacted with CDI and amine R₄NH₂ to yield 303, which is reacted with chlorocarbonyl sulfenylchloride to yield thiadiazolidinedione 304. Conversion of 304 to the substituted hydrazine 305 is accomplished by standard procedures. Hydrazine 305 can be converted into compounds of formula I.B using the methods previously outlined in Schemes 35 and 36.

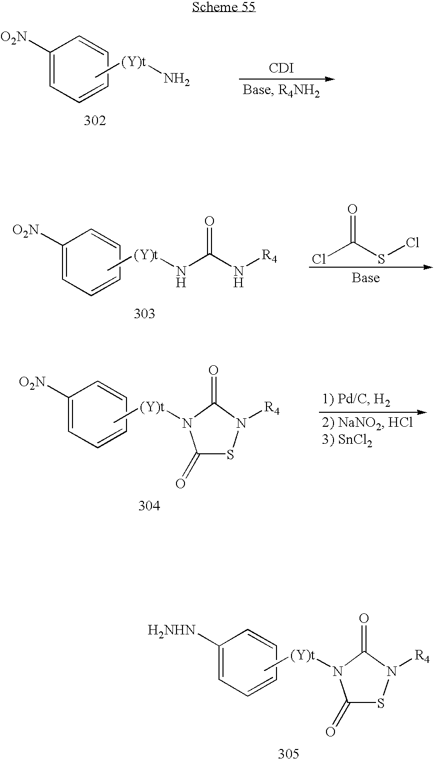

Scheme 56 illustrates an approach to the preparation of compounds of formula I.B wherein Q is Q-37, Q-38 or Q-39. Imides 309a, 309b, and 312 are all available via several literature methods, and are each able to be alkylated with chloride 306 to yields intermediates 307, 310 and 313 respectively. Intermediates 307, 310 and 313 are respectively converted to hydrazines 308 (Q=Q-37), 311 (Q=Q-38), and 314 (Q=Q-39) by standard procedures.

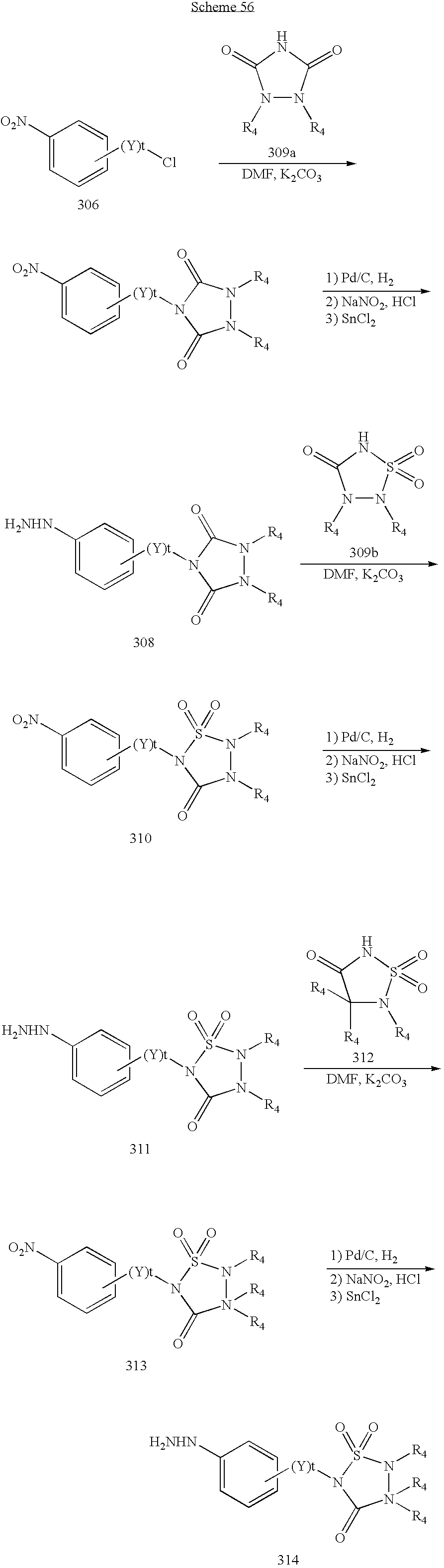

Scheme 57 illustrates an alternative preparation of compounds wherein Q is Q-39. Readily available amine 315, wherein P is a suitable amine-protecting group or a group convertible to an amine group, is reacted with SO₂Cl₂ to give rise to sulfonyl chloride 316. Intermediate 316 is reacted with a substituted amino acid ester with a suitable base to afford sulfonylurea 317. Further treatment with base results in cyclization to afford sulfohydantoin 318. The protecting group P is removed to afford the key amine-containing intermediate 319. Alternatively, if P is a nitro group, then 318 is converted to 319 under reducing conditions such as iron/HCl, tin(II) chloride, or catalytic hydrogenation. Amine 319 is converted to 320A by reaction with an isocyanate; 319 is converted to amide 320B by reaction with an acid chloride, acid anhydride, or a suitable activated carboxylic acid in the presence of a suitable base; 319 is converted to carbamate 320C by reaction with a substituted alkyl or aryl chloroformate in the presence of a suitable base.

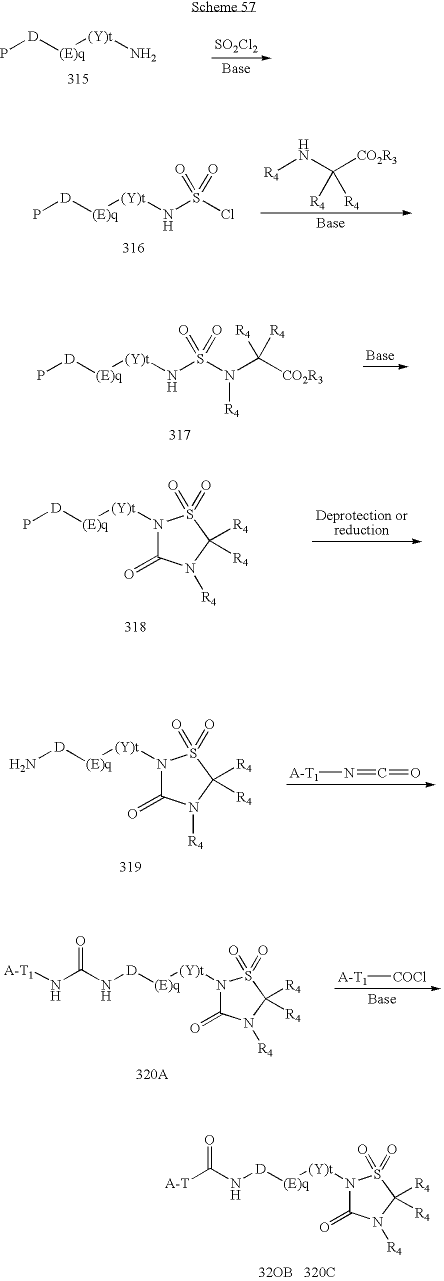

Scheme 58 illustrates an alternative synthesis of key substituted hydrazine 325 of compounds wherein Q is Q-39. This hydrazine can be converted into compounds of formula I.B using the methods previously outlined in Schemes 35 and 36. The amine 321 is reacted with SO₂Cl₂ to give rise to sulfonyl chloride 322. Reaction of 322 with a suitable amino acid ester affords sulfonylurea 323, which is cyclized under basic conditions to give sulfohydantoin 324. Reduction of the nitro group of 324, diazotization of the resulting amine, and reduction of the diazonium salt affords key hydrazine 325.

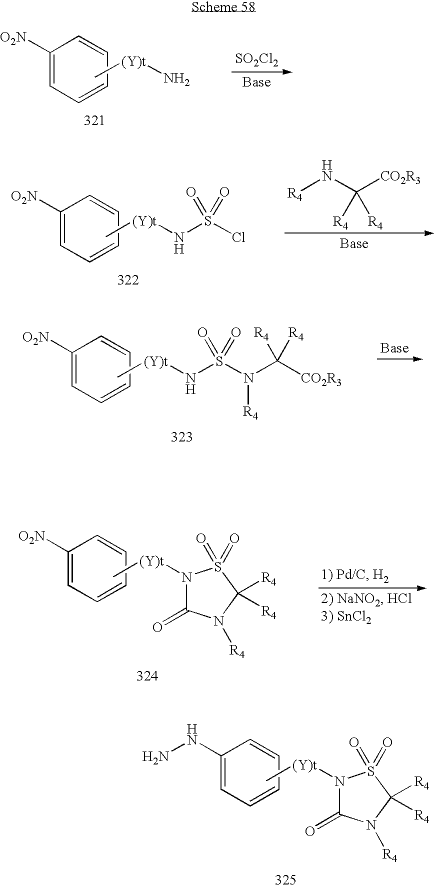

Scheme 59 illustrates an alternative preparation of compounds wherein Q is Q-38. Readily available amine 326, wherein P is a suitable amine-protecting group or a group convertible to an amine group, is reacted with SO₂Cl₂ to give rise to sulfonyl chloride 327. Intermediate 327 is reacted with a substituted hydrazide ester with a suitable base to afford sulfonylurea 328. Further treatment with base results in cyclization to afford sulfotriazaolinedione 329. The protecting group P is removed to afford the key amine-containing intermediate 330. Alternatively, if P is a nitro group, then 329 is converted to 330 under reducing conditions such as iron/HCl, tin(II) chloride, or catalytic hydrogenation. Amine 330 is converted to 331A by reaction with an isocyanate; 330 is converted to amide 331B by reaction with an acid chloride, acid anhydride, or a suitable activated carboxylic acid in the presence of a suitable base; 330 is converted to carbamate 331C by reaction with a substituted alkyl or aryl chloroformate in the presence of a suitable base.

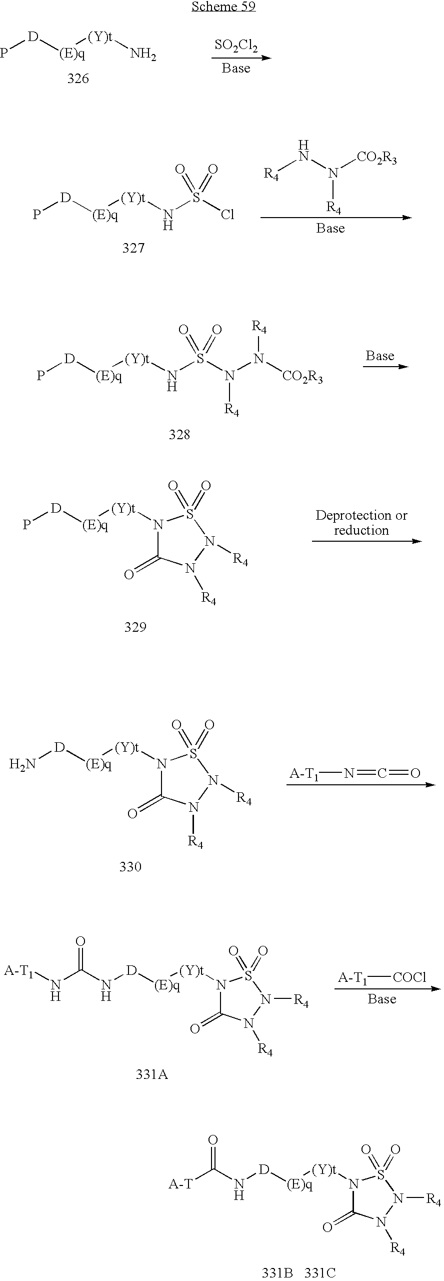

Scheme 60 illustrates an alternative synthesis of key substituted hydrazine 336 of compounds wherein Q is Q-38. This hydrazine can be converted into compounds of formula I.B using the methods previously outlined in Schemes 35 and 36. The amine 332 is reacted with SO₂Cl₂ to give rise to sulfonyl chloride 333. Reaction of 333 with a substituted hydrazide ester affords sulfonylurea 334, which is cyclized under basic conditions to give sulfotriazaolinedione 335. Reduction of the nitro group of 335, diazotization of the resulting amine, and reduction of the diazonium salt affords key hydrazine 336.

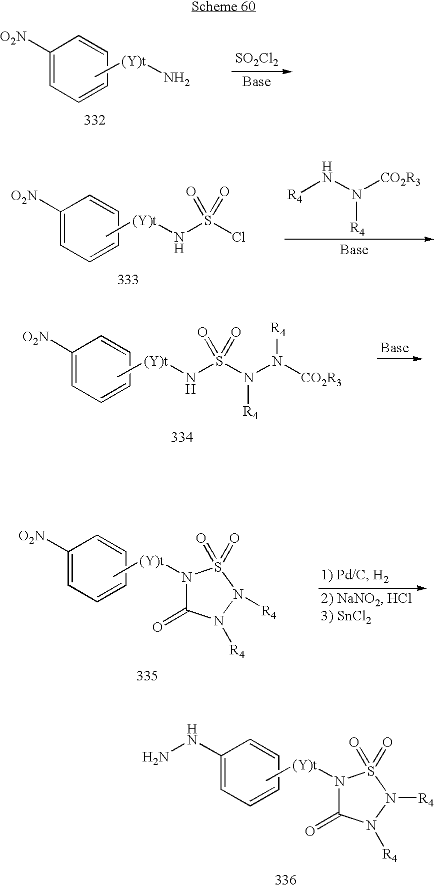

Scheme 61 illustrates the preparation of compounds wherein Q is Q-37. Readily available amine 337, wherein P is a suitable amine-protecting group or a group convertible to an amine group, is reacted with p-nitrophenyl chloroformate to give rise to carbamate 338. Intermediate 338 is reacted with a substituted amino acid ester with a suitable base to afford urea 339. Further treatment with base results in cyclization to afford triazolinedione 340. The protecting group P is removed to afford the key amine-containing intermediate 341. Alternatively, if P is a nitro group, then 340 is converted to 341 under reducing conditions such as iron/HCl, tin(II) chloride, or catalytic hydrogenation. Amine 341 is converted to 342A by reaction with an isocyanate; 341 is converted to amide 342B by reaction with an acid chloride, acid anhydride, or a suitable activated carboxylic acid in the presence of a suitable base; 341 is converted to carbamate 342C by reaction with a substituted alkyl or aryl chloroformate in the presence of a suitable base.

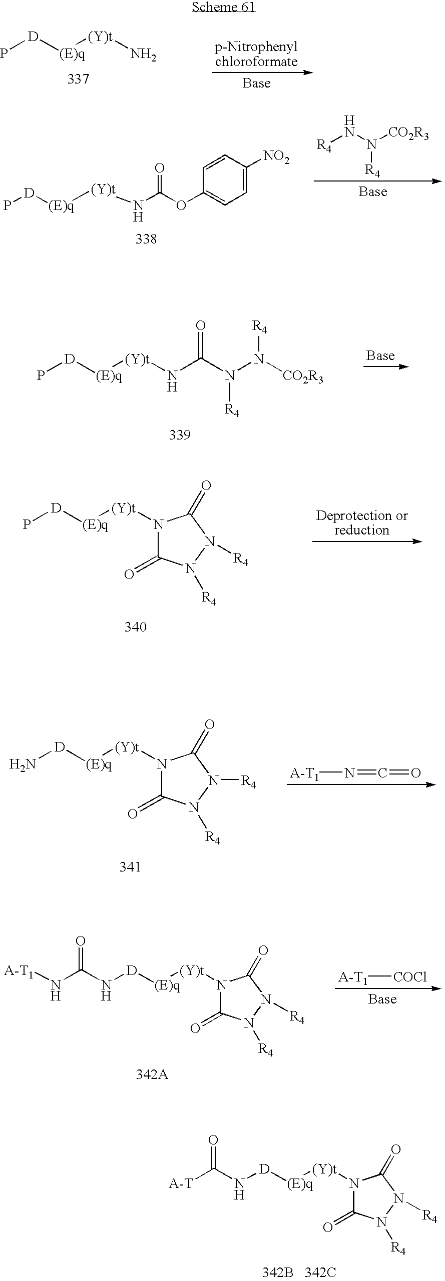

Scheme 62 illustrates an alternative synthesis of key substituted hydrazine 347 of compounds wherein Q is Q-37. This hydrazine can be converted into compounds of formula I.B using the methods previously outlined in Schemes 35 and 36. The nitrophenyl substituted amine 343 is reacted with p-nitrophenyl chloroformate to give rise to carbamate 344. Reaction of 344 with a suitable amino acid ester affords urea 345, which is cyclized under basic conditions to give triazolinedione 346. Reduction of the nitro group of 346, diazotization of the resulting amine, and reduction of the diazonium salt affords key hydrazine 347.

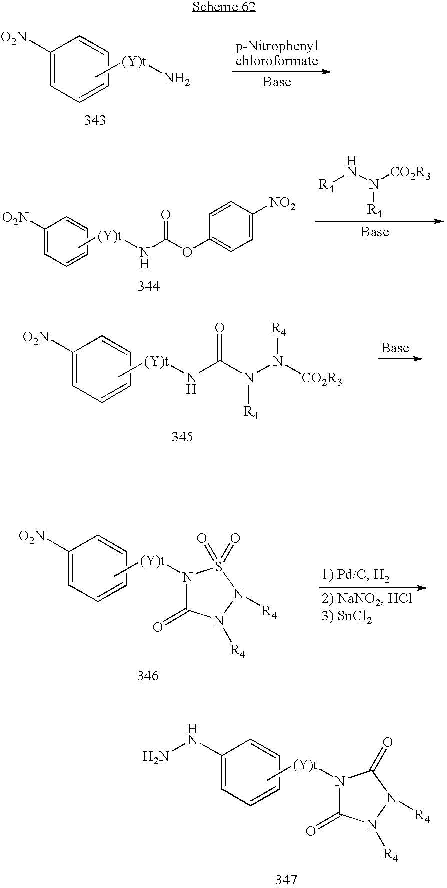

Scheme 63 illustrates the synthesis of compounds wherein Q is Q-43. Morphiline 348 is alkylated with protected bromohydrine. Removal of the alcohol protecting group yields intermediate 349, which can be oxidized to aldehyde 350. When G=NH, iodoaniline 351 is reacted with 350 under reductive amination conditions, preferably sodium triacetoxyborohydride, to afford intermediate 352. This intermediate is converted to the substituted hydrazine 353 by Cu(I)-catalyzed reaction with N—BOC hydrazine. When G=O, iodophenol 355 is either alkylated with 354 or reacted under Mitsunobu conditions with alcohol 349 to yield intermediate 356. This intermediate is converted to the substituted hydrazine 353 by Cu(I)-catalyzed reaction with N—BOC hydrazine.

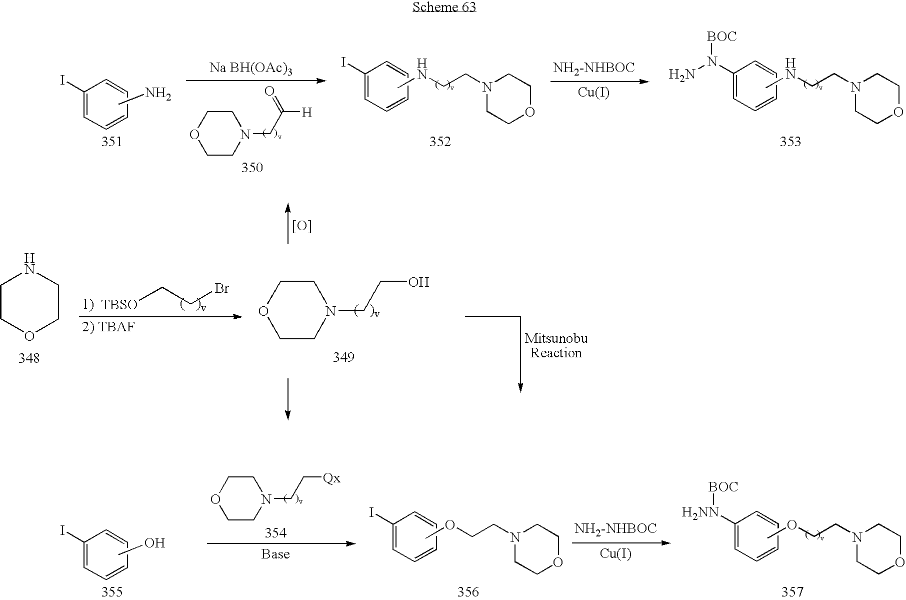

Scheme 64 illustrates the synthesis of compounds wherein Q is Q-43, G=CH₂. Nitroacid 358 (readily available by anyone with normal skills in the art) is reacted with morphiline to yield amide 359 which upon reduction to the amine and conversion of the nitro group under standard conditions results in hydrazine 360. This hydrazine can be converted into compounds of formula I.B using the methods previously outlined in Schemes 35 and 36.

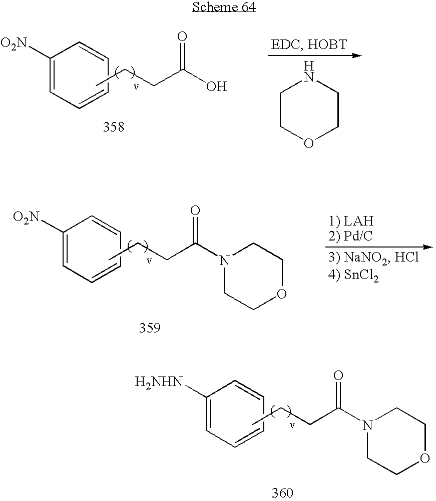

Scheme 65 illustrates the synthesis of compounds wherein Q is Q-44. N-methyl piperazine 361 is alkylated with protected bromohydrine. Removal of the alcohol protecting group yields intermediate 362, which can be oxidized to aldehyde 363. When G=NH, iodoaniline 364 is reacted with 363 under reductive amination conditions, preferably sodium triacetoxyborohydride, to afford intermediate 365. This intermediate is converted to the substituted hydrazine 366 by Cu(I)-catalyzed reaction with N—BOC hydrazine. When G=O, iodophenol 368 is either alkylated with 367 or reacted under Mitsunobu conditions with alcohol 362 to yield intermediate 369. This intermediate is converted to the substituted hydrazine 370 by Cu(I)-catalyzed reaction with N—BOC hydrazine.

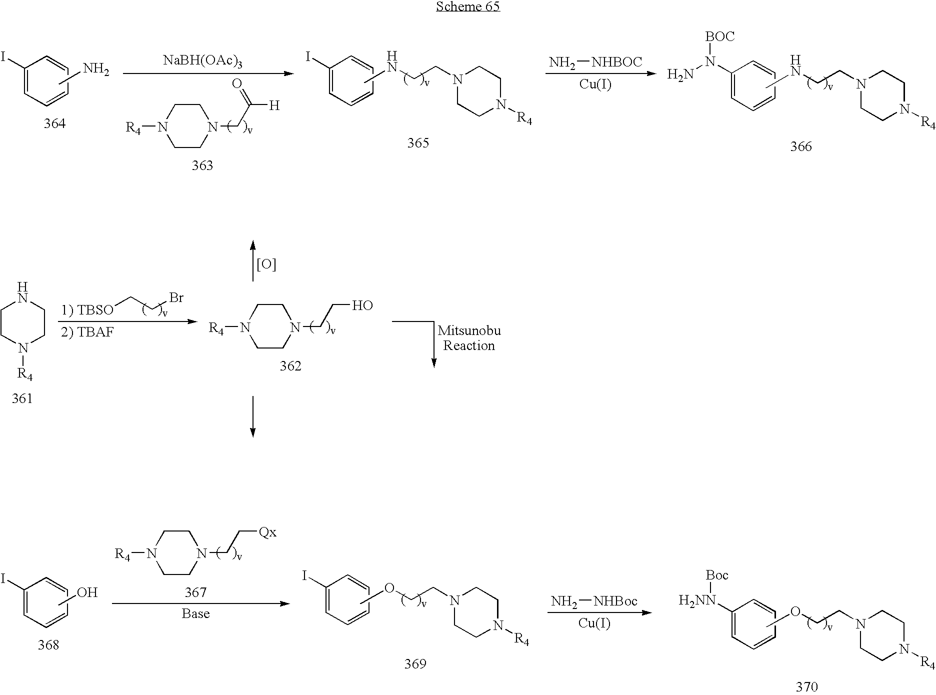

Scheme 66 illustrates the synthesis of compounds wherein Q is Q-44, G=CH₂. Nitroacid 371 (readily available by anyone with normal skills in the art) is reacted with N-methyl piperazine to yield amide 372, which upon reduction to the amine and conversion of the nitro group under standard conditions results in hydrazine 373. This hydrazine can be converted into compounds of formula I.B using the methods previously outlined in Schemes 35 and 36.

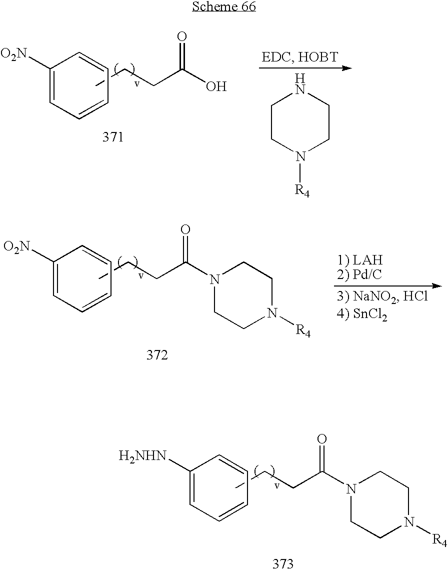

Scheme 67 illustrates the synthesis of compounds wherein Q is Q-45. Thiomorpholine sulphone 374 is alkylated with protected bromohydrine. Removal of the alcohol protecting group yields intermediate 375, which can be oxidized to aldehyde 376. When G=NH, iodoaniline 377 is reacted with 376 under reductive amination conditions, preferably sodium triacetoxyborohydride, to afford intermediate 378. This intermediate is converted to the substituted hydrazine 379 by Cu(I)-catalyzed reaction with N—BOC hydrazine. When G=O, iodophenol 380 is either alkylated with 381 or reacted under Mitsunobu conditions with alcohol 375 to yield intermediate 382. This intermediate is converted to the substituted hydrazine 383 by Cu(I)-catalyzed reaction with N—BOC hydrazine.

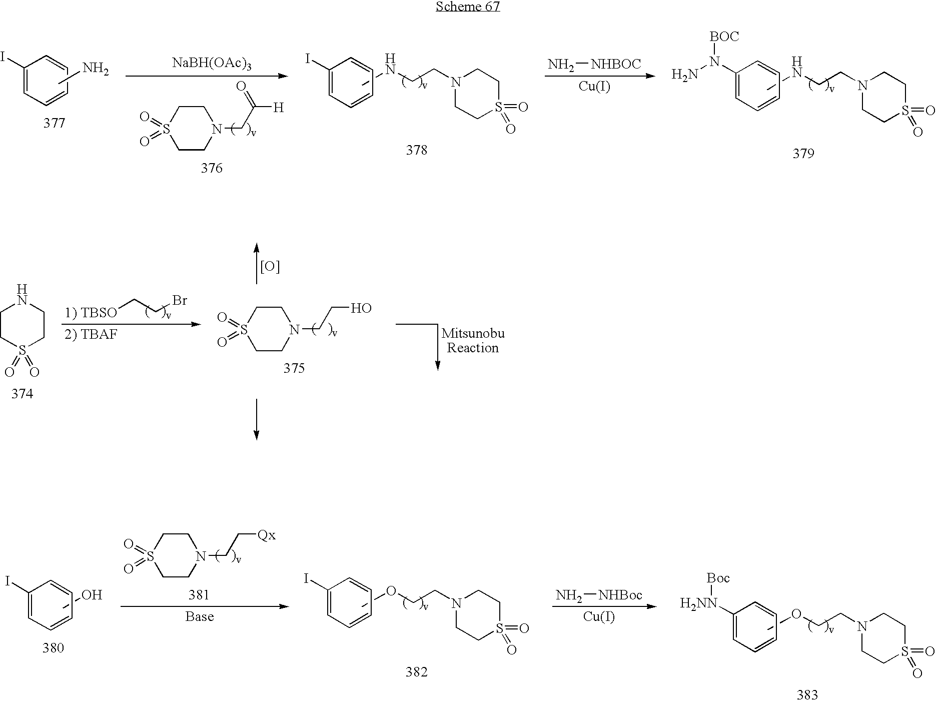

Scheme 68 illustrates the synthesis of compounds wherein Q is Q-45, G=CH₂. Nitroacid 384 (readily available by anyone with normal skills in the art) is reacted with thiomorpholine sulphone to yield amide 385, which upon reduction to the amine and conversion of the nitro group under standard conditions results in hydrazine 386. This hydrazine can be converted into compounds of formula I.B using the methods previously outlined in Schemes 35 and 36.

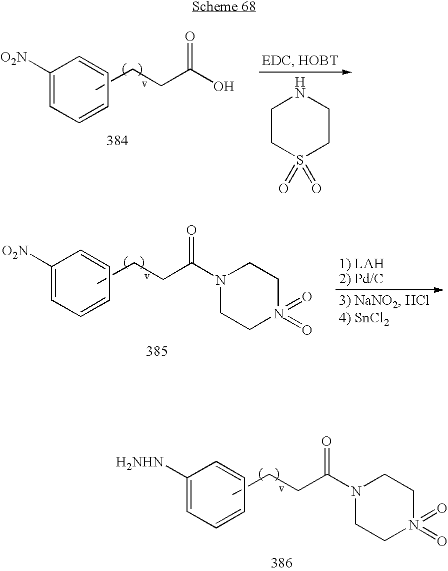

Scheme 69 illustrates the synthesis of compounds wherein Q is Q-46. Piperadine derivative 387 is alkylated with protected bromohydrine. Removal of the alcohol protecting group yields intermediate 388, which can be oxidized to aldehyde 389. When G=NH, iodoaniline 390 is reacted with 389 under reductive amination conditions, preferably sodium triacetoxyborohydride, to afford intermediate 391. This intermediate is converted to the substituted hydrazine 392 by Cu(I)-catalyzed reaction with N—BOC hydrazine. When G=O, iodophenol 393 is either alkylated with 396 or reacted under Mitsunobu conditions with alcohol 388 to yield intermediate 394. This intermediate is converted to the substituted hydrazine 395 by Cu(I)-catalyzed reaction with N—BOC hydrazine.

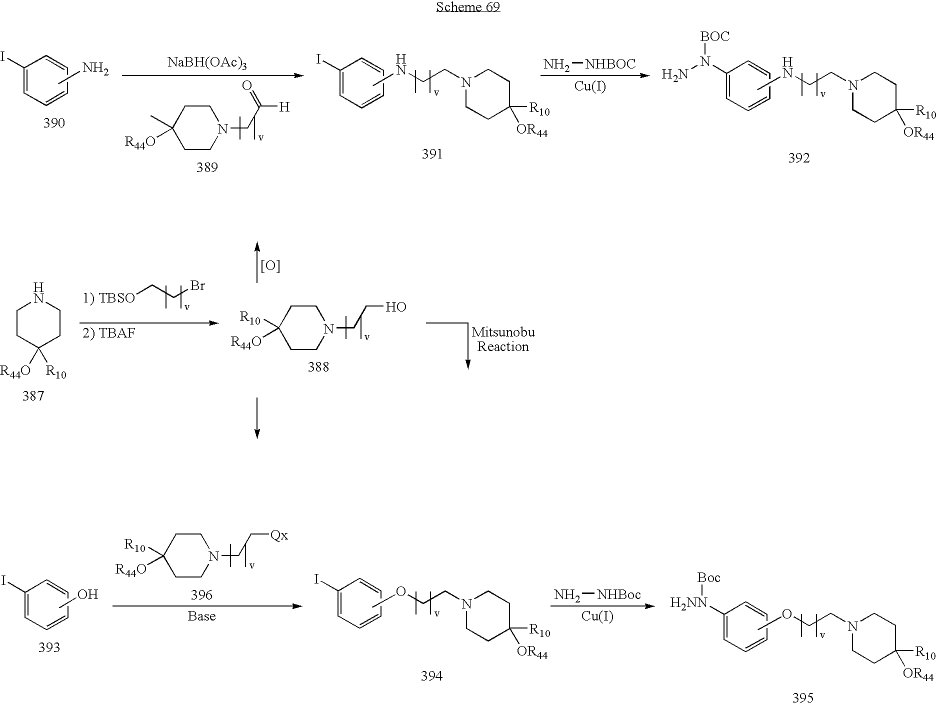

Scheme 70 illustrates the synthesis of compounds wherein Q is Q-46, G=CH₂. Nitroacid 397 (readily available by anyone with normal skills in the art) is reacted with thiomorpholine sulphone to yield amide 398, which upon reduction to the amine and conversion of the nitro group under standard conditions results in hydrazine 399. This hydrazine can be converted into compounds of formula I.B using the methods previously outlined in Schemes 35 and 36.

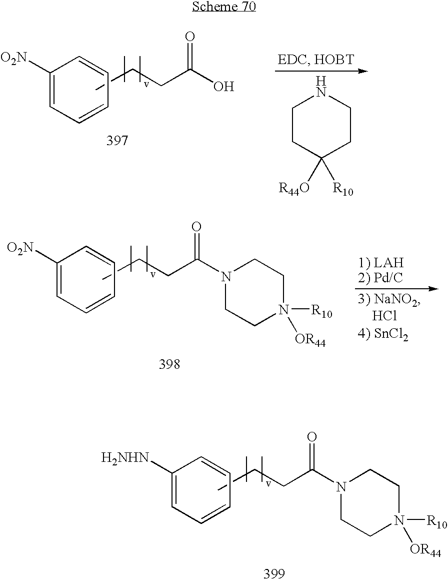

### Affinity and Biological Assessment of P38-Alpha Kinase Inhibitors

A fluorescence binding assay is used to detect binding of inhibitors of Formula I with unphosphorylated p38-alpha kinase as previously described: see J. Regan et al, Journal of Medicinal Chemistry (2002) 45:2994.

1. P38 MAP Kinase Binding Assay

The binding affinities of small molecule modulators for p38 MAP kinase were determined using a competition assay with SKF 86002 as a fluorescent probe, modified based on published methods (C. Pargellis, et al Nature Structural Biology (2002) 9, 268-272. J. Regan, et al J. Med. Chem. (2002) 45, 2994-3008). Briefly, SKF 86002, a potent inhibitor of p38 kinase (K_{d}=180 nM), displays an emission fluorescence around 420 nm when excitated at 340 nm upon its binding to the kinase. Thus, the binding affinity of an inhibitor for p38 kinase can be measured by its ability to decrease the fluorescence from SKF 86002. The assay was performed in a 384 plate (Greiner uclear 384 plate) on a Polarstar Optima plate reader (BMG). Typically, the reaction mixture contained 1 μM SKF 86002, 80 nM p38 kinase and various concentrations of an inhibitor in 20 mM Bis-Tris Propane buffer, pH 7, containing 0.15% (w/v) n-octylglucoside and 2 mM EDTA in a final volume of 65 μl. The reaction was initiated by addition of the enzyme. The plate was incubated at room temperature (˜25° C.) for 2 hours before reading at emission of 420 nm and excitation at 340 nm. By comparison of rfu (relative fluorescence unit) values with that of a control (in the absence of an inhibitor), the percentage of inhibition at each concentration of the inhibitor was calculated. IC₅₀ value for the inhibitor was calculated from the % inhibition values obtained at a range of concentrations of the inhibitor using Prism. When time-dependent inhibition was assessed, the plate was read at multiple reaction times such as 0.5, 1, 2, 3, 4 and 6 hours. The IC₅₀ values were calculated at the each time point. An inhibition was assigned as time-dependent if the IC₅₀ values decrease with the reaction time (more than two-fold in four hours). This is illustrated below in Table 1.

TABLE 1

| Example # | IC50, nM | Time-dependent |
|---|---|---|
| 1 | 292 | Yes |
| 2 | 997 | No |
| 2 | 317 | No |
| 3 | 231 | Yes |
| 4 | 57 | Yes |
| 5 | 1107 | No |
| 6 | 238 | Yes |
| 7 | 80 | Yes |
| 8 | 66 | Yes |
| 9 | 859 | No |
| 10 | 2800 | No |
| 11 | 2153 | No |
| 12 | ˜10000 | No |
| 13 | 384 | Yes |
| 15 | 949 | No |
| 19 | ˜10000 | No |
| 21 | 48 | Yes |
| 22 | 666 | No |
| 25 | 151 | Yes |
| 26 | 68 | Yes |
| 29 | 45 | Yes |
| 30 | 87 | Yes |
| 31 | 50 | Yes |
| 32 | 113 | Yes |
| 37 | 497 | No |
| 38 | 508 | No |
| 41 | 75 | Yes |
| 42 | 373 | No |
| 43 | 642 | No |
| 45 | 1855 | No |
| 46 | 1741 | No |
| 47 | 2458 | No |
| 48 | 3300 | No |
| 57 | 239 | Yes |

IC50 values obtained at 2 hours reaction time

P-38 Alpha Kinase Assay (Spectrophometric Assay)

Activity of phosphorylated p-38 kinase was determined by following the production of ADP from the kinase reaction through coupling with the pyruvate kinase/lactate dehydrogenase system (e.g., Schindler, et al. Science (2000) 289, 1938-1942). In this assay, the oxidation of NADH was continuously measured spectrophometrically. The reaction mixture (100 μl) contained phospho p-38 alpha kinase (3.3 nM. Panvera), peptide substrate (IPTSPITTTYFFFKKK-OH, 0.2 mM), ATP (0.3 mM), MgCl₂ (10 mM), pyruvate kinase (8 units. Sigma), lactate dehydrogenase (13 units. Sigma), phosphoenol pyruvate (1 mM), and NADH (0.28 mM) in 65 mM Tris buffer, pH 7.5, containing 3.5% DMSO and 150 uM n-Dodecyl-B-D-maltopyranoside. The reaction was initiated by adding ATP. The absorption at 340 nm was monitored continuously for up to 4 hours at 30° C. on Polarstar Optima plate reader (BMG). The kinase activity (reaction rate) was calculated from the slope at the time frame from 1.5 h to 2 h. Under these conditions, a turn over number (k_{cat}) of ˜1 s⁻¹ was obtained. The reaction rates calculated from different time frames such as 0.5 min to 0.5 h, 0.5 h to 1 h, 1.5 h to 2 h or 2.5 h to 3 h were generally constant.

For inhibition determinations, test compounds were incubated with the reaction mixture for ˜5 min before adding ATP to start the reaction. Percentage of inhibition was obtained by comparison of reaction rate with that of a control well containing no test compound. IC₅₀ values were calculated from a series of % inhibition values determined at a range of concentrations of each inhibitor using Prism to process the data and fit inhibition curves. Generally, the rates obtained at the time frame of 1.5 h to 2 h were used for these calculations. In assessing whether inhibition of a test compound was time-dependent (i.e., greater inhibition with a longer incubation time), the values of % inhibition and/or IC50 values obtained from other time frames were also calculated for the inhibitor. The biological activity for compounds of the present invention in the spectrophotometric assay are illustrated in Tables 2 and 3.

TABLE 2

| Example # | IC50, uM | % inhibition @ concentration, uM |
|---|---|---|
| 1 | 0.067 | |
| 2 | 0.29 | |
| 3 | 0.019 | |
| 4 | 0.609 | |
| 5 | 0.514 | |
| 6 | 0.155 | |
| 7 | 0.165 | |
| 9 | 0.355 | |
| 10 | | 83% @ 10 |
| 11 | 0.953 | |
| 12 | | 70% @ 10 |
| 13 | 0.269 | |
| 14 | 0.096 | |
| 15 | 0.53 | |
| 17 | | 40% @ 10 |
| 18 | | 60% @ 10 |
| 21 | 0.171 | |
| 22 | 0.445 | |
| 25 | 0.055 | |
| 26 | 0.19 | |
| 29 | 0.011 | |
| 30 | 0.251 | |
| 31 | 0.056 | |
| 32 | 0.307 | |
| 38 | 0.51 | |
| 39 | 0.012 | |
| 40 | 0.055 | |
| 41 | 0.013 | |
| 42 | 0.425 | |
| 43 | 7.5 | |
| 45 | 0.48 | |
| 46 | 1 | |
| 47 | 0.295 | |
| 48 | 2 | |
| 49 | 0.071 | |
| 51 | 0.033 | |
| 52 | 0.416 | |
| 53 | 0.109 | |
| 54 | | 68% @ 1.0 |
| 55 | 0.74 | |
| 57 | 0.782 | |
| 58 | 0.172 | |
| 59 | 0.709 | |
| 60 | 0.264 | |
| D | 0.179 | |
| F | 0.437 | |

TABLE 3

| Example # | IC50, uM | % Inhibition @ concentration, uM |
|---|---|---|
| 145 | 1.3 | |
| 146 | | 9% @ 10 |
| 147 | | 27% @ 10 |
| 150 | | 53% @ 10 |
| 154 | | 21% @ 10 |
| 155 | | 58% @ 10 |
| 160 | 0.044 | |
| 161 | 0.1 | |
| 162 | 0.65 | |
| 163 | 0.464 | |
| 196 | 0.028 | |
| 197 | 0.243 | |
| 198 | 0.137 | |
| 199 | 0.684 | |
| 200 | | 73% @ 1.0 |
| 201 | 0.029 | |
| 202 | 1.9 | |
| 203 | 0.328 | |
| 204 | 0.008 | |
| 206 | 0.013 | |
| 207 | 0.033 | |
| 209 | 0.354 | |
| 234 | 11 | |
| 284 | 1.95 | |
| 285 | 0.102 | |
| 286 | 0.079 | |
| 287 | 0.041 | |
| 288 | 0.104 | |
| 289 | 1.3 | |
| 291 | 5.1 | |
| 294 | 2.1 | |
| 295 | 1.2 | |
| 296 | 0.284 | |
| 297 | 0.34 | |
| 298 | 0.025 | |
| 299 | 2.3 | |
| 300 | 0.251 | |
| 301 | 0.63 | |
| 302 | 0.077 | |

| Example | uM |
|---|---|
| 303 | 0.089 |
| 306 | 0.058 |
| 307 | 0.049 |
| 308 | 0.12 |
| 309 | 0.30 |
| 310 | 0.13 |
| 311 | 0.182 |
| 312 | 0.349 |
| 313 | 0.25 |
| 314 | 0.25 |
| 315 | 54% @ 10 |
| 316 | 0.42 |
| 317 | 0.068 |
| 318 | 0.67 |
| 319 | 0.32 |
| 320 | 0.79 |
| 321 | 0.52 |
| 322 | 2.02 |
| 323 | 51% @ 10, 9% @ 1 |
| 324 | 40% @ 10 |
| 325 | 54% @ 10 |
| 326 | 41% @ 10 |
| 327 | 0.81 |
| 328 | 68% @ 10 |
| 329 | 0.25 |
| 330 | 2.1 |
| 331 | 71% @ 10 |
| 332 | 0.05 |
| 333 | 2.438 |
| 334 | 2.2 |
| 335 | 0.014 |
| 336 | 27% @ 10 |
| 337 | 8% @ 10 |
| 338 | 7% @ 2 |
| 339 | 9% @ 2 |
| 340 | 31% @ 10 |
| 342 | 1 |
| 344 | 0.18 |
| 345 | 0.27 |
| 346 | 19% @ 0.1, 63% @ 1 |
| 347 | 8% @ 0.1, 31% @ 1 |
| 348 | 17% @ 0.1, 47% @ 1 |
| 350 | 39% @ 0.1, 64% @ 1 |
| 352 | 0.043 |
| 354 | 0.11 |
| 355 | 0.042 |
| 356 | 0.059 |
| 357 | 0.13 |
| 358 | 0.1 |
| 359 | 0.013 |
| 360 | 0.39 |
| 361 | 0.87 |
| 363 | 0.19 |
| 364 | 0.01 |
| 365 | 0.007 |
| 366 | 53% @ 10 |
| 367 | 85% @ 10 |
| 369 | 0.00 |
| 372 | 1.46 |
| 374 | 0.037 |
| 376 | 0.48 |
| 378 | 0.400 |
| 380 | 2.00 |
| 382 | 0.88 |
| 383 | 0.1 |
| 384 | 96% @ 10, 72% @ 1 |
| 385 | 0.03 |
| 386 | 0.1138 |
| 387 | 4.446 |
| 388 | 1.645 |
| 389 | 0.09 |
| 390 | 24% @ 0.1, 63% @ 1 |
| 391 | 0.91 |
| 392 | 75% @ 0.1, 86% @ 1 |
| 393 | 0.4 |
| 395 | 0.056 |
| 396 | 3% @ 0.1, 56% @ 1 |
| 397 | 0.04 |
| 399 | 21% @ 0.1, 74% @ 1 |
| 404 | −11% @ 0.1, 48% @ 1 |
| 406 | 0.01343 |
| 407 | 0.01032 |
| 408 | 0.1165 |
| 409 | 0.03089 |
| 411 | 0.008 |
| 532 | 66% @ 10 |
| 415 | 28% @ 0.1, 69% @ 1 |
| 416 | 67% @ 0.1, 83% @ 1 |
| 418 | 0.075 |
| 420 | 0.0147 |
| 421 | 0.0147 |
| 426 | 0.013 |
| 427 | 0.819 |
| 428 | 0.1403 |
| 429 | 1.068 |
| 430 | 0.9424 |
| 432 | 24% @ 10, 9% @ 1 |
| 434 | 1.9 |
| 436 | 16% @ 0.1, 41% @ 1 |
| 438 | 1.3 |
| 440 | 0.92 |
| 442 | 3.01 |
| 444 | 5.00 |
| 450 | 192% @ 10, 89% @ 1 |
| 451 | 3% @ 1 |
| 455 | 0.10 |
| 456 | 0.04 |
| 457 | 1.1 |
| 458 | 1.0 |
| 459 | 0.02 |
| 461 | 0.017 |
| 463 | 0.056 |
| 464 | 0.019 |
| 465 | 0.24 |
| 466 | 0.007 |
| 467 | 0.119 |
| 468 | 0.0086 |
| 470 | 0.005 |
| 472 | 0.050 |
| 473 | 0.004 |
| 474 | 0.38 |
| 475 | 0.01 |
| 476 | 0.024 |
| 477 | 0.042 |
| 480 | 0.15 |
| 481 | 0.23 |
| 482 | 0.084 |
| 483 | 0.27 |
| 484 | 0.019 |
| 485 | 0.050 |
| 486 | 0.057 |
| 487 | 0.038 |
| 488 | 0.54 |
| 489 | 0.52 |
| 490 | 0.34 |
| 491 | 0.34 |
| 492 | 0.04 |
| 493 | 73% @ 10, 36% @ 1 |
| 494 | 0.67 |
| 495 | 0.25 |
| 498 | 25% @ 1 |
| 499 | 78% @ 0.1, 95% @ 1 |
| 506 | 0.042 |
| 507 | 0.007 |
| 508 | 0.052 |
| 509 | 0.5 |
| 510 | 0.47 |
| 511 | 0.024 |
| 512 | 0.032 |
| 513 | 0.41 |
| 514 | 0.57 |
| 516 | 0.45 |
| 519 | 0.052 |
| 520 | 0.0092 |
| 522 | 0.78 |
| 525 | 11% @ 1 |
| 526 | 25% @ 0.1, 70% @ 1 |
| 527 | 0.0033 |
| 528 | 35% @ 1 |
| 529 | 40% @ 10 |
| 530 | 83% @ 10 |
| 531 | 14% @ 10 |
| 533 | 39% @ 10 |

Human Peripheral Blood Mononuclear Leukocyte Cell Assay.

Human peripheral blood mononuclear leukocytes are challenged with 25 ng/mL lipopolysaccharide (LPS) in the absence or presence of Test Compound and incubated for 16 hours as described by Welker P. et al, International Archives Allergy and Immunology (1996) 109: 110. The quantity of LPS-induced tumor necrosis factor-alpha (TNF-alpha) cytokine release is measured by a commercially available Enzyme-Linked Immunosorbent Assay (ELISA) kit. Test compounds are evaluated for their ability to inhibit TNF-alpha release. Table 2 records IC₅₀ values for inhibition of TNF-alpha release by Test Compounds of the present invention, wherein the IC₅₀ value, in micromolar concentration, represents the concentration of Test Compound resulting in a 50% inhibition of TNF-alpha release from human peripheral blood mononuclear leukocytes as compared to control experiments containing no Test Compound. Test compounds evaluated are illustrated in Table 4.

TABLE 4

| Example Number | IC50, uM |
|---|---|
| 3 | 6.1 |
| 13 | 6.32 |
| 21 | 3.4 |
| 29 | 2.68 |
| 31 | 4.52 |
| 60 | 2.34 |
| 296 | 3.49 |
| 300 | 4.78 |
| 302 | 5.45 |

### EXAMPLES

The following examples set forth preferred methods in accordance with the invention. It is to be understood, however, that these examples are provided by way of illustration and nothing therein should be taken as a limitation upon the overall scope of the invention.

[Boc-sulfamide] aminoester (Reagent AA), 1,5,7,-trimethyl-2,4-dioxo-3-aza-bicyclo[3.3.1]nonane-7-carboxylic acid (Reagent BB), and Kemp acid anhydride (Reagent CC) was prepared according to literature procedures. See Askew et. al J. Am. Chem. Soc. 1989, 111, 1082 for further details.

### Example A

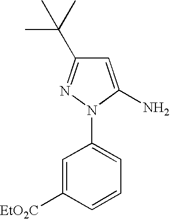

To a solution (200 mL) of m-amino benzoic acid (200 g, 1.46 mol) in concentrated HCl was added an aqueous solution (250 mL) of NaNO₂ (102 g, 1.46 mol) at 0° C. The reaction mixture was stirred for 1 h and a solution of SnCl₂.2H₂O (662 g, 2.92 mol) in concentrated HCl (2 L) was then added at 0° C., and the reaction stirred for an additional 2h at RT. The precipitate was filtered and washed with ethanol and ether to yield 3-hydrazino-benzoic acid hydrochloride as a white solid.

The crude material from the previous reaction (200 g, 1.06 mol) and 4,4-dimethyl-3-oxo-pentanenitrile (146 g, 1.167 mol) in ethanol (2 L) were heated to reflux overnight. The reaction solution was evaporated in vacuo and the residue purified by column chromatography to yield ethyl 3-(3-tert-butyl-5-amino-1H-pyrazol-1-yl)benzoate (Example A, 116 g, 40%) as a white solid together with 3-(5-amino-3-tert-butyl-1H-pyrazol-1-yl)benzoic acid (93 g, 36%). ¹H NMR (DMSO-d₆): 8.09 (s, 1H), 8.05 (brd, J=8.0 Hz, 1H), 7.87 (brd, J=8.0 Hz, 1H), 7.71 (t, J=8.0 Hz, 1H), 5.64 (s, 1H), 4.35 (q, J=7.2 Hz, 2H), 1.34 (t, J=7.2 Hz, 3H), 1.28 (s, 9H).

### Example B

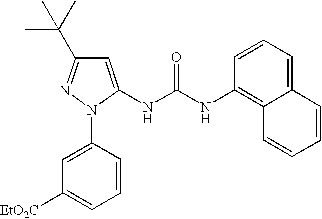

To a solution of 1-naphthyl isocyanate (9.42 g, 55.7 mmol) and pyridine (44 mL) in THF (100 mL) was added a solution of Example A (8.0 g, 27.9 mmol) in THY (200 mL) at 0° C. The mixture was stirred at RT for 1 h, heated until all solids were dissolved, stirred at RT for an additional 3 h and quenched with H₂O (200 mL). The precipitate was filtered, washed with dilute HCl and H₂O, and dried in vacuo to yield ethyl 3-[3-t-butyl-5-(3-naphthalen-1-yl)ureido)-1H-pyrazol-1-yl]benzoate (12.0 g, 95%) as a white power. ¹H NMR (DMSO-d₆): 9.00 (s, 1H), 8.83 (s, 1H), 8.25 7.42 (m, 11H), 6.42 (s, 1H), 4.30 (q, J=7.2 Hz, 2H), 1.26 (s, 9H), 1.06 (t, J=7.2 Hz, 3H); MS (ESI) m/z: 457.10 (M+H⁺).

### Example C

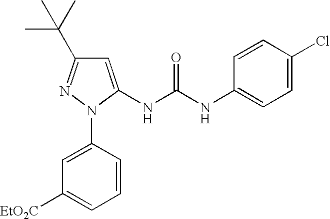

To a solution of Example A (10.7 g, 70.0 mmol) in a mixture of pyridine (56 mL) and THF (30 mL) was added a solution of 4-nitrophenyl 4-chlorophenylcarbamate (10 g, 34.8 mmol) in THF (150 mL) at 0° C. The mixture was stirred at RT for 1 h and heated until all solids were dissolved, and stirred at RT for an additional 3 h. H₂O (200 mL) and CH₂Cl₂ (200 mL) were added, the aqueous phase separated and extracted with CH₂Cl₂ (2×100 mL). The combined organic layers were washed with 1N NaOH, and 0.1N HCl, saturated brine and dried over anhydrous Na₂SO₄. The solvent was removed in vacuo to yield ethyl 3-{3-tert-butyl-5-[3-(4-chlorophenyl)ureido]-1H-pyrazol-1-yl}benzoate (8.0 g, 52%). ¹H NMR (DMSO-d₆): δ 9.11 (s, 1H), 8.47 (s, 1H), 8.06 (m, 1H), 7.93 (d, J=7.6 Hz, 1H), 7.81 (d, J=8.0 Hz, 1H), 7.65 (dd, J=8.0, 7.6 Hz, 1H), 7.43 (d, J=8.8 Hz, 2H), 7.30 (d, J=8.8 Hz, 2H), 6.34 (s, 1H), 4.30 (q, J=6.8 Hz, 2H), 1.27 (s, 9H), 1.25 (t, J=6.8 Hz, 3H); MS (ESI) m/z: 441 (M⁺+H).

### Example D

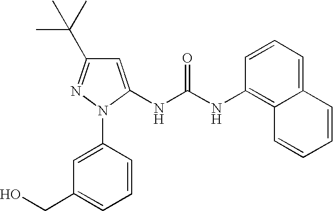

To a stirred solution of Example B (8.20 g, 18.0 mmol) in THF (500 mL) was added LiAlH₄ powder (2.66 g, 70.0 mmol) at −10° C. under N₂. The mixture was stirred for 2 h at RT and excess LiAlH₄ destroyed by slow addition of ice. The reaction mixture was acidified to pH=7 with dilute HCl, concentrated in vacuo and the residue extracted with EtOAc. The combined organic layers were concentrated in vacuo to yield 1-{3-tert-butyl-1-[3-(hydroxymethyl)phenyl]-1H-pyrazol-5-yl}-3-(naphthalen-1-yl)urea (7.40 g, 99%) as a white powder. ¹H NMR (DMSO-d₆): 9.19 (s,1H), 9.04 (s, 1H), 8.80 (s,1H), 8.26-7.35 (m, 1H), 6.41 (s, 1H), 4.60 (s, 2H), 1.28 (s, 9H); MS (ESI) m/z: 415 (M+H⁺).

### Example E

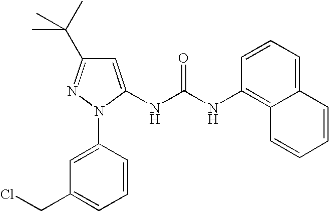

A solution of Example C (1.66 g, 4.0 mmol) and SOCl₂ (0.60 mL, 8.0 mmol) in CH₃Cl (100 mL) was refluxed for 3 h and concentrated in vacuo to yield 1-{3-tert-butyl-1-[3-chloromethyl)phenyl]-1H-pyrazol-5-yl}-3-(naphthalen-1-yl)urea (1.68 g, 97%) was obtained as white powder. ¹H NMR (DMSO-d₆): δ 9.26 (s, 1H), 9.15 (s, 1H), 8.42-7.41 (m, 11H), 6.40 (s, 1H), 4.85 (s, 2H), 1.28 (s, 9H). MS (ESI) m/z: 433 (M+H⁺).

### Example F

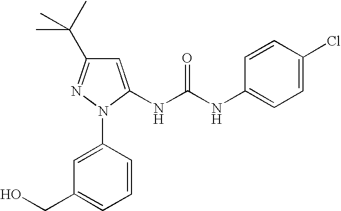

To a stirred solution of Example C (1.60 g, 3.63 mmol) in THF (200 mL) was added LiAlH₄ powder (413 mg, 10.9 mmol) at −10° C. under N₂. The mixture was stirred for 2 h and excess LiAlH₄ was quenched by adding ice. The solution was acidified to pH=7 with dilute HCl. Solvents were slowly removed and the solid was filtered and washed with EtOAc (200+100 mL). The filtrate was concentrated to yield 1-{3-tert-butyl-1-[3-hydroxymethyl)phenyl]-1H-pyrazol-5-yl}-3-(4-chlorophenyl)urea (1.40 g, 97%).¹H NMR (DMSO-d₆): δ 9.11 (s, 1H), 8.47 (s, 1H), 7.47-7.27 (m, 8H), 6.35 (s, 1H), 5.30 (t, J=5.6 Hz, 1H), 4.55 (d, J=5.6 Hz, 2H), 1.26 (s, 9H); MS (ESI) m/z: 399 (M+H⁺).

### Example G

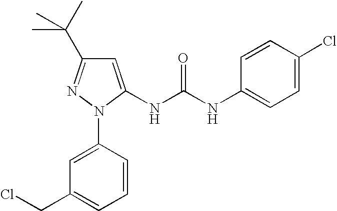

A solution of Example F (800 mg, 2.0 mmol) and SOCl₂ (0.30 mL, 4 mmol) in CHCl₃ (30 mL) was refluxed gently for 3 h. The solvent was evaporated in vacuo and the residue was taken up to in CH₂Cl₂ (2×20 mL). After removal of the solvent, 1-{3-tert-butyl-1-[3-(chloromethyl)phenyl]-1H-pyrazol-5-yl}-3-(4-chlorophenyl)urea (812 mg, 97%) was obtained as white powder. ¹H NMR (DMSO-d₆): δ 9.57 (s, 1H), 8.75 (s, 1H), 7.63 (s, 1H), 7.50-7.26 (m, 7H), 6.35 (s, 1H), 4.83 (s, 2H), 1.27 (s, 9H); MS (ESI) m/z: 417 (M+H⁺).

### Example H

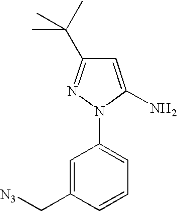

To a suspension of LiAlH₄ (5.28 g, 139.2 mmol) in THF (1000 mL) was added Example A (20.0 g, 69.6 mmol) in portions at 0° C. under N₂. The reaction mixture was stirred for 5 h, quenched with 1 N HCl at 0° C. and the precipitate was filtered, washed by EtOAc and the filtrate evaporated to yield [3-(5-amino-3-tert-butyl-1H-pyrazol-1-yl)phenyl]methanol (15.2 g, 89%). ¹H NMR (DMSO-d₆): 7.49 (s, 1H), 7.37 (m, 2H), 7.19 (d, J=7.2 Hz, 1H), 5.35 (s, 1H), 5.25 (t, J=5.6 Hz, 1H), 5.14 (s, 2H), 4.53 (d, J=5.6 Hz, 2H), 1.19 (s, 9H); MS (ESI) m/z: 246.19 (M+H⁺).

The crude material from the previous reaction (5.0 g, 20.4 mmol) was dissolved in dry THF (50 mL) and SOCl₂ (4.85 g, 40.8 mmol), stirred for 2 h at RT, concentrated in vacuo to yield 3-tert-butyl-1-(3-chloromethylphenyl)-1H-pyrazol-5-amine (5.4 g), which was added to N₃ (3.93 g, 60.5 mmol) in DMF (50 mL). The reaction mixture was heated at 30° C. for 2 h, poured into H₂O (50 mL), and extracted with CH₂Cl₂. The organic layers were combined, dried over MgSO₄, and concentrated in vacuo to yield crude 3-tert-butyl-1-[3-(azidomethyl)phenyl]-1H-pyrazol-5-amine (1.50 g, 5.55 mmol).

### Example I

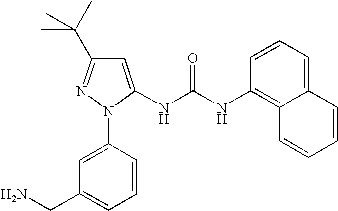

Example H was dissolved in dry THF (10 mL) and added a THF solution (10 mL) of 1-isocyano naphthalene (1.13 g, 6.66 mmol) and pyridine (5.27 g, 66.6 mmol) at RT. The reaction mixture was stirred for 3 h, quenched with H₂O (30 mL), the resulting precipitate filtered and washed with 1N HCl and ether to yield 1-[2-(3-azidomethyl-phenyl)-5-t-butyl-2H-pyrazol-3-yl]-3-naphthalen-1-yl-urea (2.4 g, 98%) as a white solid.

The crude material from the previous reaction and Pd/C (0.4 g) in THF (30 mL) was hydrogenated under 1 atm at RT for 2 h. The catalyst was removed by filtration and the filtrate concentrated in vacuo to yield 1-{3-tert-butyl-1-[3-(aminomethyl)phenyl}-1H-pyrazol-5yl)-3-(naphthalene-1-yl)urea (2.2 g, 96%) as a yellow solid. ¹H NMR (DMSO-d₆): 9.02 (s, 1H), 7.91 (d, J=7.2 Hz, 1H), 7.89(d, J=7.6 Hz, 2H), 7.67-7.33 (m, 9H), 6.40 (s, 1H), 3.81 (s, 2H), 1.27 (s, 9H); MS (ESI) m/z: 414 (M+H⁺).

### Example J

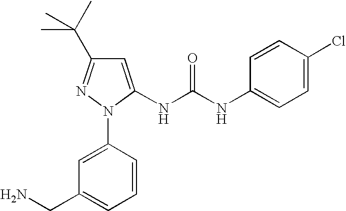

To a solution of Example H (1.50 g, 5.55 mmol) in dry THF (10 mL) was added a THF solution (10 mL) of 4-chlorophenyl isocyanate (1.02 g, 6.66 mmol) and pyridine (5.27 g, 66.6 mmol) at RT. The reaction mixture was stirred for 3 h and then H₂O (30 mL) was added. The precipitate was filtered and washed with 1N HCl and ether to give 1-{3-tert-butyl-1-[3-(aminomethyl)phenyl}-1H-pyrazol-5yl)-3-(4-chlorophenyl)urea (2.28 g, 97%) as a white solid, which was used for next step without further purification. MS (ESI) m/z: 424 (M+H⁺).

### Example K

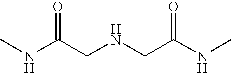

To a solution of benzyl amine (16.5 g, 154 mmol) and ethyl bromoacetate (51.5 g, 308 mmol) in ethanol (500 mL) was added K₂CO₃ (127.5 g, 924 mmol). The mixture was stirred at RT for 3 h, was filtered, washed with EtOH, concentrated in vacuo and chromatographed to yield N-(2-ethoxy-2-oxoethyl)-N-(phenylmethyl)-glycine ethyl ester (29 g, 67%). ¹H NMR (CDCl₃): δ 7.39-7.23 (m, 5H), 4.16 (q, J=7.2 Hz, 4H), 3.91(s, 2H), 3.54 (s, 4H), 1.26 (t, J=7.2 Hz, 6H); MS (ESI): m/e: 280 (M⁺+H).

A solution of N-(2-ethoxy-2-oxoethyl)-N-(phenylmethyl)-glycine ethyl ester (7.70 g, 27.6 mmol) in methylamine alcohol solution (25-30%, 50 mL) was heated to 50° C. in a sealed tube for 3 h, cooled to RT and concentrated in vacuo to yield N-(2-methylamino-2-oxoethyl)-N-(phenylmethyl)-glycine methylamide in quantitative yield (7.63 g). ¹H NMR (CDCl₃): δ 7.35-7.28 (m, 5H), 6.75 (br s, 2H), 3.71(s, 2H), 3.20 (s, 4H), 2.81 (d, J=5.6 Hz, 6H); MS (ESI) m/e 250(M+H⁺).

The mixture of N-(2-methylamino-2-oxoethyl)-N-(phenylmethyl)-glycine methylamide (3.09 g, 11.2 mmol) in MeOH (30 mL) was added 10% Pd/C (0.15 g). The mixture was stirred and heated to 40° C. under 40 psi H₂ for 10 h, filtered and concentrated in vacuo to yield N-(2-methylamino-2-oxoethyl)-glycine methylamide in quantitative yield (1.76 g). ¹H NMR (CDCl₃): δ 6.95(br s, 2H), 3.23 (s, 4H), 2.79 (d, J=6.0, 4.8 Hz), 2.25(br s 1H); MS (ESI) m/e 160(M+H⁺)

### Example 1

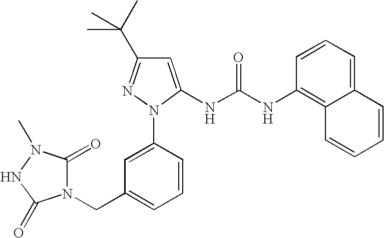

To a solution of 1-methyl-[1,2,4]triazolidine-3,5-dione (188 mg, 16.4 mmol) and sodium hydride (20 mg, 0.52 mmol) in DMSO (1 mL) was added Example E (86 mg, 0.2 mmol). The reaction was stirred at RT overnight, quenched with H₂O (10 mL), extracted with CH₂Cl₂, and the organic layer was separated, washed with brine, dried over Na₂SO₄ and concentrated in vacuo. The residue was purified by preparative HPLC to yield 1-(3-tert-butyl-1-{3-[(1-methyl-3,5-dioxo-1,2,4-triazolidin-4-yl)methyl]phenyl}-1H-pyrazol-5-yl)-3-(naphthalene-1-yl)urea (Example 1, 14 mg). ¹H NMR (CD₃OD): δ7.88-7.86 (m, 2H), 7.71-7.68 (m, 2H), 7.58 (m, 2H), 7.60-7.42 (m, 5H), 6.49 (s, 1H), 4.85 (s, 1H), 1.34 (s, 9H), 1.27 (s, 6H); MS (ESI) m/z: 525 (M+H⁺).

### Example 2

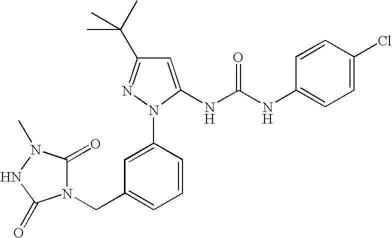

The title compound was synthesized in a manner analogous to Example 1, utilizing Example G to yield 1-(3-tert-butyl-1-{3-[(1-methyl-3,5-dioxo-1,2,4-triazolidin-4-yl)methyl]phenyl}-1H-pyrazol-5-yl)-3-(4-chlorophenyl)urea ¹H NMR (CD₃OD): δ 7.2-7.5 (m, 7H), 6.40 (s 1H), 4.70 (s, 2H), 2.60 (d, J=14 Hz, 2H), 1.90 (m, 1H), 1.50 (m, 1H), 1.45 (s, 9H), 1.30 (m, 2H), 1.21 (s, 3H), 1.18 (s, 6H); MS (ESI) m/z: 620 (M+H⁺).

### Example 3

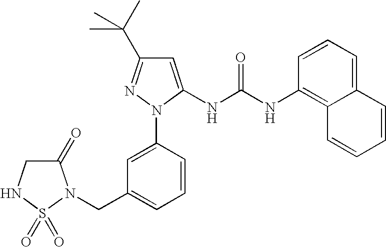

A mixture of compound 1,1-Dioxo-[1,2,5]thiadiazolidin-3-one (94 mg, 0.69 mmol) and NaH (5.5 mg, 0.23 mmol) in THF (2 mL) was stirred at −10° C. under N₂ for 1 h until all NaH was dissolved. Example E (100 mg, 0.23 mmol) was added and the reaction was allowed to stir at RT overnight, quenched with H₂O, and extracted with CH₂Cl₂. The combined organic layers were concentrated in vacuo and the residue was purified by preparative HPLC to yield 1-(3-tert-butyl-1-{[3-(1,1,3-trioxo-[1,2,5]thiadiazolidin-2-yl)methyl]phenyl}-1H-pyrazol-5-yl)-3-(naphthalen-1-yl)urea (18 mg) as a white powder. ¹H NMR (CD₃OD): δ 7.71-7.44 (m, 11H), 6.45 (s, 1H), 4.83 (s, 2H), 4.00 (s, 2H), 1.30 (s, 9H). MS (ESI) m/z: 533.40 (M+H⁺).

### Example 4

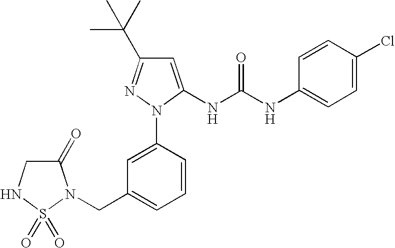

The title compound was obtained in a manner analogous to Example 3 utilizing Example G. to yield 1-(3-tert-butyl-1-{[3-(1,1,3-trioxo-[1,2,5]thiadiazolidin-2-yl)methyl]phenyl}-1H-pyrazol-5-yl)-3-(4-chlorophenyl)urea. ¹H NMR (CD₃OD): δ 7.38-7.24 (m, 8H), 6.42 (s, 1H), 4.83 (s, 2H), 4.02 (s, 2H), 1.34 (s, 9H); MS (ESI) m/z: 517 (M+H⁺).

### Example 5

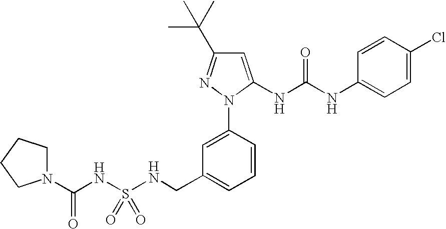

To a stirred solution of chlorosulfonyl isocyanate (19.8 μL, 0.227 mmol) in CH₂Cl₂ (0.5 mL) at 0° C. was added pyrrolidine (18.8 μL, 0.227 mmol) at such a rate that the reaction solution temperature did not rise above 5° C. After stirring for 1.5 h, a solution of Example J (97.3 mg, 0.25 mmol) and Et₃N (95 μL, 0.678 mmol) in CH₂Cl₂ (1.5 mL) was added at such a rate that the reaction temperature didn rise above 5° C. When the addition was completed, the reaction solution was warmed to RT and stirred overnight. The reaction mixture was poured into 10% HCl, extracted with CH₂Cl₂, the organic layer washed with saturated NaCl, dried over MgSO₄, and filtered. After removal of the solvents, the crude product was purified by preparative HPLC to yield 1-(3-tert-butyl-1-[[3-N-[[(1-pyrrolidinylcarbonyl)amino]sulphonyl]aminomethyl]phenyl]-1H-pyrazol-5-yl)-3-(4-chlorophenyl)urea. ¹H NMR(CD₃OD): δ 7.61 (s, 1H), 7.43-7.47 (m, 3H), 7.23-7.25 (dd, J=6.8 Hz, 2H), 7.44 (dd, J=6.8 Hz, 2H), 6.52 (s, 1H), 4.05 (s, 2H), 3.02 (m, 4H), 1.75 (m, 4H), 1.34 (s, 9H); MS (ESI) m/z: 574.00 (M+H⁺).

### Example 6

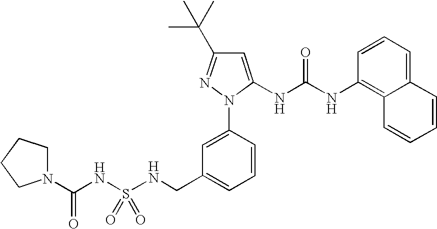

The title compound was made in a manner analogous to Example 5 utilizing Example I to yield 1-(3-tert-butyl-1-[[3-N-[[(1-pyrrolidinylcarbonyl)amino]sulphonyl]aminomethyl]-phenyl]-1H-pyrazol-5-yl)-3-(naphthalen-1-yl)urea. ¹HNMR (CDCl₃): δ 7.88 (m, 2H), 7.02-7.39 (m, 2H), 7.43-7.50(m, 7H), 6.48(s, 1H), 4.45(s, 1H), 3.32-3.36 (m, 4H), 1.77-1.81 (m, 4H), 1.34 (s,9H); MS (ESI) m/z: 590.03 (M+H⁺).

### Example 7

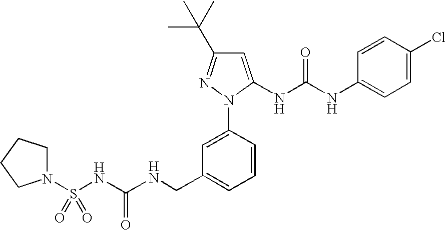

To a stirred solution of chlorosulfonyl isocyanate (19.8 μΛ, 0.227 μμoλ) τν XH₁Xλ₁ (0.5 μΛ) ατ 0° C., was added Example J (97.3 mg, 0.25 mmol) at such a rate that the reaction solution temperature did not rise above 5° C. After being stirred for 1.5 h, a solution of pyrrolidine (18.8 μL, 0.227 mmol) and Et₃N (95 μL, 0.678 mmol) in CH₂Cl₂ (1.5 mL) was added at such a rate that the reaction temperature didn rise above 5° C. When addition was completed, the reaction solution was warmed to RT and stirred overnight. The reaction mixture was poured into 10% HCl, extracted with CH₂Cl₂, the organic layer was washed with saturated NaCl, dried over Mg₂SO₄, and filtered. After removal of the solvents, the crude product was purified by preparative HPLC to yield 1-(3-tert-butyl-1-[[3-N-[[(1-pyrrolidinylsulphonyl)amino]carbonyl]aminomethyl]phenyl]-1H-pyrazol-5-yl)-3-(4-chlorophenyl)urea. ¹HNMR (CDCl₃): δ 7.38 (m, 1H), 7.36-7.42 (m, 3H), 7.23 (d, J=8.8 Hz, 2H), 7.40 (d, J=8.8 Hz, 2H), 6.43 (s, 1H), 4.59 (s,1H), 4.43 (s, 2H), 1.81 (s, 2H), 1.33 (s, 9H); MS (ESI) m/z: 574.10 (M+H⁺).

### Example 8

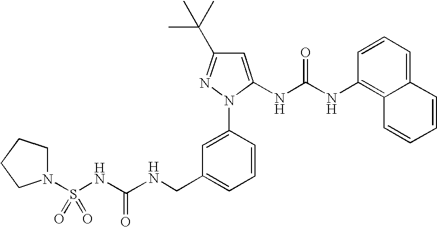

The title compound was made in a manner analogous to Example 7 utilizing Example I to yield 1-(3-tert-butyl-1-[[3-N-[[(1-pyrrolidinylsulphonyl)amino]carbonyl]aminomethyl]-phenyl]-1H-pyrazol-5-yl)-3-(naphthalen-1-yl)urea. ¹HNMR (CDCl₃): δ 7.88 (m, 2H), 7.02-7.39 (m, 2H), 7.43-7.50 (m, 7H), 6.48 (s, 1H), 4.45 (s, 1H), 3.32-3.36 (m, 4H), 1.77-1.81 (m, 4H), 1.34 (s,9H); MS (ESI) m/z: 590.03 (M+H⁺).

### Example 9

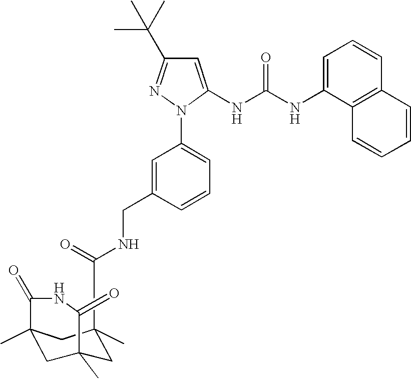

To a solution of Reagent BB (36 mg, 0.15 mmol), Example 1 (62 mg, 0.15 mmol), HOBt (40 mg, 0.4 mmol) and NMM (0.1 mL, 0.9 mmol) in DMF (10 mL) was added EDCI (58 mg, 0.3 mmol). After being stirred overnight, the mixture was poured into water (15 mL) and extracted with EtOAc (3 5 mL). The organic layers were combined, washed with brine, dried with Na₂SO₄, and concentrated in vacuo. The residue was purified by preparative TLC to yield 1,5,7-trimethyl-2,4-dioxo-3-azabicyclo[3.3.1]nonane-7-carboxylic acid 3-[3-t-butyl-5-(3-naphthalen-1-yl-ureido)-pyrazol-1-yl]benzylamide (22 mg). ¹HNMR (CDCl₃): δ 8.40 (s, 1H), 8.14 (d, J=8.0 Hz, 2H), 7.91 (s, 1H), 7.87 (s, 1H), 7.86 (d, J=7.2 Hz, 1H), 7.78 (d, J=7.6 Hz, 1H), 7.73 (d, J=8.4 Hz, 1H), 7.69 (d, J=8.4 Hz, 1H), 7.57-7.40 (m, 4H), 7.34 (d, J=7.6 Hz, 1H), 6.69 (s, 1H), 6.32 (t, J=5.6 Hz, 1H), 5.92 (brs, 1H), 4.31 (d, J=5.6 Hz, 2H), 2.37 (d, J=14.8 Hz, 2H), 1.80 (d, J=13.2 Hz, 1H), 1.35 (s, 9H), 1.21 (d, J=13.2 Hz, 1H), 1.15 (s, 3H), 1.12 (d, J=12.8 Hz, 2H), 1.04 (s, 6H); MS (ESI) m/z: 635 (M+H⁺).

### Example 10

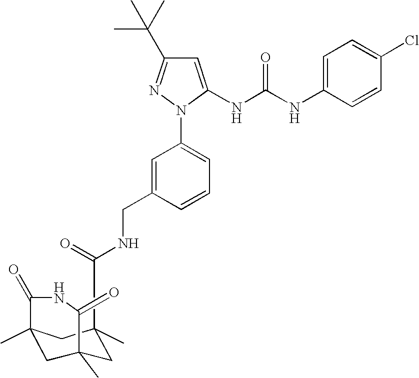

The title compound, was synthesized in a manner analogous to Example 9 utilizing Example J to yield 1,5,7-trimethyl-2,4-dioxo-3-aza-bicyclo[3.3.1]nonane-7-carboxylic acid 3-{3-t-butyl-5-[3-(4-chloro-phenyl)-ureido]-pyrazol-1-yl}benzylamide. ¹H NMR (CDCl₃): δ 8.48 (s, 1H), 7.78 (s, 1H), 7.75 (d, J=8.0 Hz, 1H), 7.69 (s, 1H), 7.53 (t, J=8.0 Hz, 1H), 7.48 (d, J=8.8 Hz, 2H), 7.26 (m, 3H), 6.62 (s, 1H), 6.35(t, J=6.0 Hz, 1H), 5.69 (brs, 1H), 4.26 (d, J=6.0 Hz, 2H), 2.48 (d, J=14.0 Hz, 2H), 1.87 (d, J=13.6 Hz, 1H), 1.35 (s, 9H), 1.25 (m, 6H), 1.15 (s, 6H); MS (ESI) m/z: 619 (M+H⁺).

### Example 11

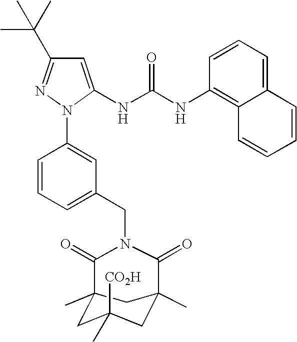

A mixture of Example 1 (41 mg, 0.1 mmol), Kemp acid anhydride (24 mg, 0.1 mmol) and Et₃N (100 mg, 1 mmol) in anhydrous CH₂Cl₂ (2 mL) were stirred overnight at RT, and concentrated in vacuo. Anhydrous benzene (20 mL) was added to the residue, the mixture was refluxed for 3 h, concentrated in vacuo and purified by preparative HPLC to yield 3-{3-[3-t-butyl-5-(3-naphthalen-1-yl-ureido)-pyrazol-1-yl]-benzyl}-1,5-di-methyl-2,4-dioxo-3-aza-bicyclo[3.3.1]nonane-7-carboxylic acid (8.8 mg, 14%). ¹H NMR (CD₃OD): δ 7.3-7.4 (m, 2H), 7.20 (m, 2H), 7.4-7.6 (m, 7H), 6.50 (m, 1H), 4.80 (s, 2H), 2.60 (d, J=14 Hz, 2H), 1.90 (m, 1H), 1.40 (m, 1H), 1.30 (m, 2H), 1.20 (s, 3H), 1.15 (s, 6H); MS (ESI) m/z: 636 (M+H⁺).

### Example 12

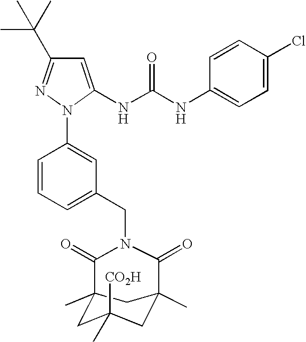

The title compound, was synthesized in a manner analogous to Example 11 utilizing Example J to yield 3-{3-[3-t-butyl-5-(3-naphthalen-1-yl-ureido)-pyrazol-1-yl]-benzyl}-1,5-dimethyl-2,4-dioxo-3-aza-bicyclo[3.3.1]nonane-7-carboxylic acid. ¹H NMR (CD₃OD): δ 7.2-7.5(m, 7H), 6.40(s 1H), 4.70(s, 2H), 2.60(d, J=14 Hz, 2H), 1.90(m, 1H), 1.50(m, 1H), 1.45 (s, 9H), 1.30 (m, 2H), 1.21 (s, 3H), 1.18 (s, 6H); MS (ESI) m/z: 620 (M+H⁺).

### Example 13

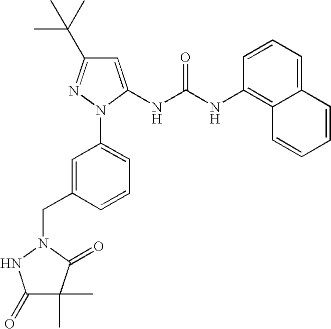

The title compound was synthesized in a manner analogous to Example 1 utilizing Example E and 4,4-dimethyl-3,5-dioxo-pyrazolidine to yield 1-(3-tert-butyl-1-{3-[(4,4-dimethyl-3,5-dioxopyrazolidin-1-yl)methyl]phenyl}-1H-pyrazol-5-yl)-3-(naphthalen-1-yl)urea. ¹HNMR (CD₃OD): δ 7.88-7.86 (m, 2H), 7.71-7.68 (m, 2H), 7.58 (m, 2H), 7.60-7.42 (m, 5H), 6.49 (s, 1H), 4.85 (s, 1H), 1.34 (s, 9H), 1.27 (s, 6H); MS (ESI) m/z: 525 (M+H⁺).

### Example 14

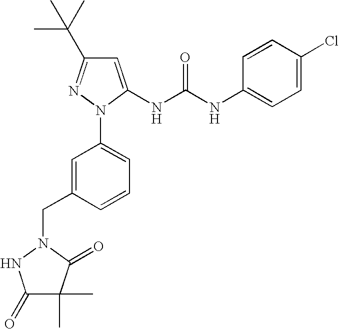

The title compound was synthesized in a manner analogous to Example 1 utilizing Example G and 4,4-dimethyl-3,5-dioxo-pyrazolidine to yield 1-(3-tert-butyl-1-{3-[(4,4-dimethyl-3,5-dioxopyrazolidin-1-yl)methyl]phenyl}-1H-pyrazol-5-yl)-3-(4-chlorophenyl)urea. ¹H NMR (CD₃OD): δ 7.60-7.20 (m, 8H), 6.43 (s, 1H), 4.70 (s, 1H), 1.34 (s, 9H), 1.26 (s, 6H); MS (ESI) m/z: 509, 511 (M+H⁺).

### Example 15

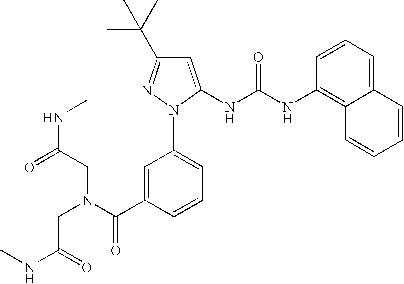

Example B was saponified with 2N LiOH in MeOH, and to the resulting acid (64.2 mg, 0.15 mmol) were added HOBt (30 mg, 0.225 mmol), Example K (24 mg, 0.15 mmol) and 4-methylmorpholine (60 mg, 0.60 mmol 4.0 equiv), DMF (3 mL) and EDCI (43 mg, 0.225 mmol). The reaction mixture was stirred at RT overnight and poured into H₂O (3 mL), and a white precipitate collected and further purified by preparative HPLC to yield 1-[1-(3-{bis[(methylcarbamoyl)methyl]carbamoyl}phenyl)-3-tert-butyl-1H-pyrazol-5-yl]-3-(naphthalen-1-yl)urea (40 mg). ¹H NMR (CDCl₃): δ 8.45 (brs, 1H), 8.10 (d, J=7.6 Hz, 1H), 7.86-7.80 (m, 2H), 7.63-7.56 (m, 2H), 7.52 (s, 1H), 7.47-7.38 (m, 3H), 7.36-7.34 (m, 1H), 7.26 (s, 1H), 7.19-7.17 (m, 2H), 6.60 (s, 1H), 3.98 (s, 2H), 3.81 (s, 3H), 2.87 (s, 3H), 2.63 (s, 3H), 1.34 (s, 9H); MS (ESI) m/z: 570 (M+H⁺).

### Example 16

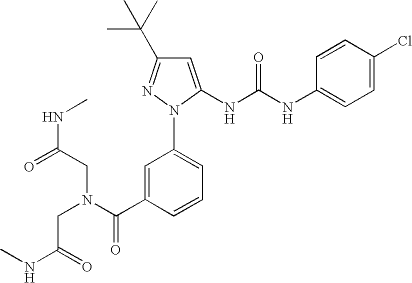

The title compound was synthesized in a manner analogous to Example 15 utilizing Example C (37 mg) and Example K to yield 1-[1-(3-{bis [(methylcarbamoyl)methyl]carbamoyl}phenyl)-3-tert-butyl-1H-pyrazol-5-yl]-3-(4-chlorophenyl)urea. ¹H NMR (CD₃OD): δ 8.58 (brs, 1H), 8.39 (brs, 1H), 7.64-7.62 (m, 3H), 7.53-7.51 (m,1H), 7.38 (d, J=9.2 Hz, 2H), 7.25 (d, J=8.8 Hz, 2H), 6.44 (s, 1H), 4.17 (s, 2H), 4.11 (s, 2H), 2.79 (s, 3H), 2.69 (s, 3H), 1.34-1.28 (m, 12H); MS (ESI) m/z: 554 (M+H⁺).

### Example 17

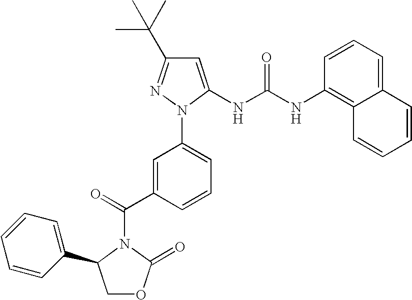

Example B was saponified with 2N LiOH in MeOH, and to the resulting acid (0.642 g, 1.5 mmol) in dry THF (25 mL) at −78° C. were added freshly distilled triethylamine (0.202 g, 2.0 mmol) and pivaloyl chloride (0.216 g, 1.80 mmol) with vigorous stirring. After stirring at −78° C. for 15 min and at 0° C. for 45 min, the mixture was again cooled to −78° C. and then transferred into the THF solution of lithium salt of D-4-phenyl-oxazolidin-2-one [*: The lithium salt of the oxazolidinone reagent was previously prepared by the slow addition of n-BuLi (2.50M in hexane, 1.20 mL, 3.0 mmol) into THF solution of D-4-phenyl-oxazoldin-2-one at −78° C.]. The reaction solution was stirred at −78° C. for 2 h and RT overnight, and then quenched with aq. ammonium chloride and extracted with dichloromethane (100 mL). The combined organic layers were dried (Na₂SO₄) and concentrated in vacuo. The residue was purified by preparative HPLC to yield D-1-{5-tert-butyl-2-[3-(2-oxo-4-phenyl-oxazolidinyl-3-carbonyl)phenyl]-2H-pyrazol-3-yl}-3-(naphthalen-1-yl)urea (207 mg, 24%). ¹HNMR(CDCl₃): δ 8.14-8.09 (m, 2H), 8.06 (s,1H), 7.86-7.81 (m, 4H), 7.79 (s, 1H), 7.68-7.61 (m, 2H), 7.51-7.40 (m, 9H), 6.75 (s, 1H), 5.80 (t, J=9.2, 7.6 Hz, 1H), 4.89 (t, J=9.2 Hz, 1H), 4.42 (dd, J=9.2, 7.6 Hz, 1H), 1.37 (s, 9H); MS (ESI) m/z: 574 (M+H⁺).

### Example 18

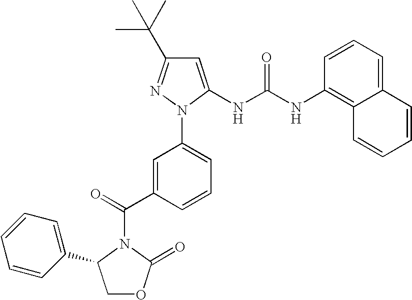

The title compound was synthesized in a manner analogous to Example 17 utilizing Example B and L-4-phenyl-oxazolidin-2-one to yield L-1-{5-tert-butyl-2-[3-(2-oxo-4-phenyl-oxazolidinyl-3-carbonyl)phenyl]-2H-pyrazol-3-yl}-3-(naphthalen-1-yl)urea ¹H NMR (CDCl₃): δ 8.14-8.09 (m, 2H), 8.06 (s,1H), 7.86-7.81 (m, 4H), 7.79 (s, 1H), 7.68-7.61 (m, 2H), 7.51-7.40 (m, 9H), 6.75 (s, 1H), 5.80 (t, J=9.2, 7.6 Hz, 1H), 4.89 (t, J=9.2 Hz, 1H), 4.42 (dd, J=9.2, 7.6 Hz, 1H), 1.37 (s, 9H); MS (ESI) m/z: 574 (M+H⁺)

### Example 19

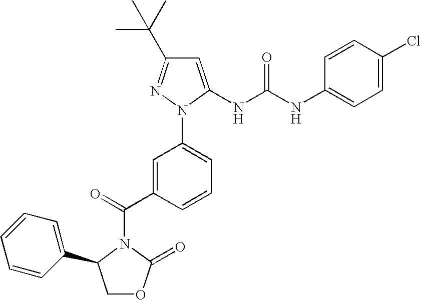

The title compound was synthesized in a manner analogous to Example 17 utilizing Example C and D-4-phenyl-oxazolidin-2-one to yield D-1-{5-tert-butyl-2-[3-(2-oxo-4-phenyl-oxazolidinyl-3-carbonyl)phenyl]-2H-pyrazol-3-yl}-3-(4-chlorophenyl)urea. ¹H NMR (CDCl₃): δ 7.91 (s, 1H), 7.85 (d, J=8.0 Hz, 1H), 7.79 (d, J=7.6 Hz, 1H), 7.71 (m, 1H), 7.65 (m, 1H), 7.49-7.40 (m, 8H), 7.26-7.24 (m, 2Hd), 6.68 (s, 1H), 5.77 (dd, J=8.8, 8.0 Hz, 1H), 4.96 (t, 8.8 Hz, 1H), 4.44 (dd, J=8.8, 8.0 Hz, 1H), 1.36 (s, 9H); MS (ESI) m/z: 558 (M+H⁺)

### Example 20

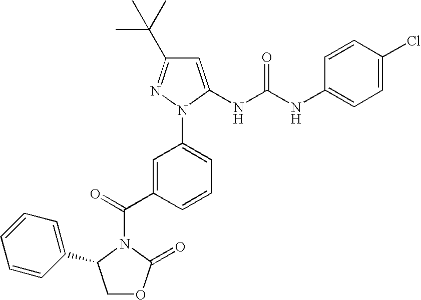

The title compound was synthesized in a manner analogous to Example 17 utilizing Example C and L-4-phenyl-oxazolidin-2-one to yield L-1-{5-tert-butyl-2-[3-(2-oxo-4-phenyl-oxazolidinyl-3-carbonyl)phenyl]-2H-pyrazol-3-yl}-3-(4-chlorophenyl)urea. ¹H NMR (CDCl₃): δ 7.91 (s, 1H), 7.85 (d, J=8.0 Hz, 1H), 7.79 (d, J=7.6 Hz, 1H), 7.71 (m, 1H), 7.65 (m, 1H), 7.49-7.40 (m, 8H), 7.26-7.24 (m, 2H), 6.68 (s, 1H), 5.77 (dd, J=8.8, 8.0 Hz, 1H), 4.96 (t, 8.8 Hz, 1H), 4.44 (dd, J=8.8, 8.0 Hz, 1H), 1.36 (s, 9H); MS (ESI) m/z: 558 (M+H⁺)

### Example L

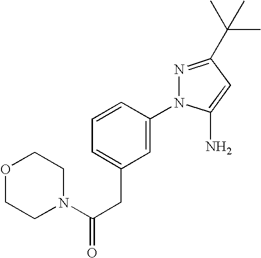

To a stirred suspension of (3-nitro-phenyl)-acetic acid (2 g) in CH₂Cl₂ (40 ml, with a catalytic amount of DMF) at 0° C. under N₂ was added oxalyl chloride (1.1 ml) drop wise. The reaction mixture was stirred for 40 min morpholine (2.5 g) was added. After stirring for 20 min, the reaction mixture was filtered. The filtrate was concentrated in vacuo to yield 1-morpholin-4-yl-2-(3-nitro-phenyl)-ethanone as a solid (2 g). A mixture of 1-morpholin-4-yl-2-(3-nitro-phenyl)-ethanone (2 g) and 10% Pd on activated carbon (0.2 g) in ethanol (30 ml) was hydrogenated at 30 psi for 3 h and filtered over Celite. Removal of the volatiles in vacuo provided 2-(3-amino-phenyl)-1-morpholin-4-yl-ethanone (1.7 g). A solution of 2-(3-amino-phenyl)-1-morpholin-4-yl-ethanone (1.7 g, 7.7 mmol) was dissolved in 6 N HCl (15 ml), cooled to 0° C., and vigorously stirred. Sodium nitrite (0.54 g) in water (8 ml) was added. After 30 min, tin (11) chloride dihydrate (10 g) in 6 N HCl (30 ml) was added. The reaction mixture was stirred at 0° C. for 3 h. The pH was adjusted to pH 14 with solid potassium hydroxide and extracted with EtOAc. The combined organic extracts were concentrated in vacuo provided 2-(3-hydrazin-phenyl)-1-morpholin-4-yl-ethanone (1.5 g). 2-(3-Hydrazinophenyl)-1-morpholin-4-yl-ethanone (3 g) and 4,4-dimethyl-3-oxopentanenitrile (1.9 g, 15 mmol) in ethanol (60 ml) and 6 N HCl (1 ml) were refluxed for 1 h and cooled to RT. The reaction mixture was neutralized by adding solid sodium hydrogen carbonate. The slurry was filtered and removal of the volatiles in vacuo provided a residue that was extracted with ethyl acetate. The volatiles were removed in vacuo to provide 2-[3-(3-tert-butyl-5-amino-1H-pyrazol-1-yl)phenyl]-1-morpholinoethanone (4 g), which was used without further purification.

### Example 21

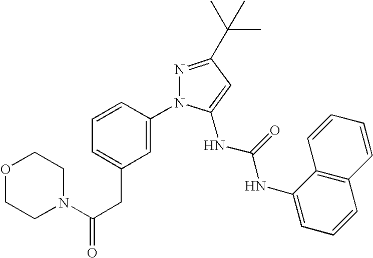

A mixture of Example L (0.2 g, 0.58 mmol) and 1-naphthylisocyanate (0.10 g, 0.6 mmol) in dry CH₂Cl₂ (4 ml) was stirred at RT under N₂ for 18 h. The solvent was removed in vacuo and the crude product was purified by column chromatography using ethyl acetate/hexane/CH₂Cl₂ (3/1/0.7) as the eluent (0.11 g, off-white solid) to yield 1-{3-tert-butyl-1-[3-(2-morpholino-2-oxoethyl)phenyl]-1H-pyrazol-5-yl}-3-(naphthalene-1-yl)urea. mp: 194-196; ¹H NMR (200 MHz, DMSO-d₆): δ 9.07 (1H, s), 8.45 (s, 1H), 8.06-7.93 (m, 3H), 7.69-7.44 (m, 7H), 7.33-7.29 (d, 6.9 Hz, 1H), 6.44 (s, 1H), 3.85 (m, 2H), 3.54-3.45 (m, 8H), 1.31 (s, 9H); MS:

### Example 22

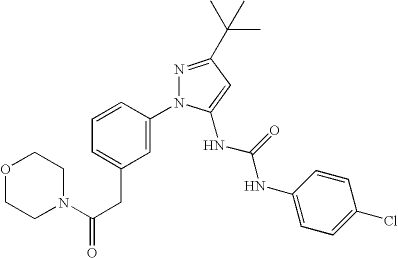

The title compound was synthesized in a manner analogous to Example 21 utilizing Example L (0.2 g, 0.58 mmol) and 4-chlorophenylisocyanate (0.09 g, 0.6 mmol) to yield 1-{3-tert-butyl-1-[3-(2-morpholino-2-oxoethyl)phenyl]-1H-pyrazol-5-yl}-3-(4-chlorophenyl)urea. mp: 100 104; ¹H NMR (200 MHz, DMSO-d₆): δ 9.16 (s, 1H), 8.45 (s, 1H), 7.52-7.30 (m, 8H), 6.38 (s, 1H), 3.83 (m, 1H), 3.53-3.46 (m, 8H), 1.30 (s, 9H); MS:

### Example 23

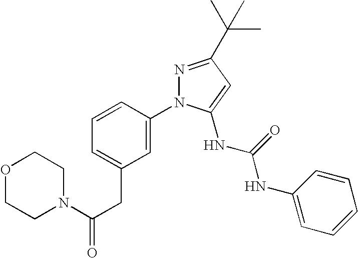

The title compound is synthesized in a manner analogous to Example 21 utilizing Example L (0.2 g, 0.58 mmol) and phenylisocyanate (0.09 g, 0.6 mmol) to yield 1-{3-tert-butyl-1-[3-(2-morpholino-2-oxoethyl)phenyl]-1H-pyrazol-5-yl}-3-phenylurea.

### Example 24

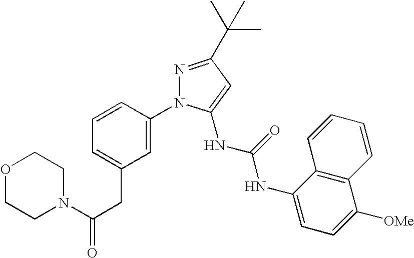

The title compound is synthesized in a manner analogous to Example 21 utilizing Example L (0.2 g, 0.58 mmol) and 1-isocyanato-4-methoxy-naphthalene to yield 1-{3-tert-butyl-1-[3-(2-morpholino-2-oxoethyl)phenyl]-1H-pyrazol-5-yl}-3-(1-methoxynaphthalen-4-yl)urea.

### Example M

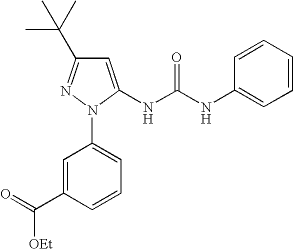

The title compound is synthesized in a manner analogous to Example C utilizing Example A and phenylisocyanate to yield ethyl 3-(3-tert-butyl-5-(3-phenylureido)-1H-pyrazol-1-yl)benzoate.

### Example N

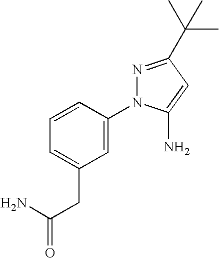

A solution of (3-nitrophenyl)acetic acid (23 g, 127 mmol) in methanol (250 ml) and a catalytic amount of concentrated in vacuo H₂SO₄ was heated to reflux for 18 h. The reaction mixture was concentrated in vacuo to a yellow oil. This was dissolved in methanol (250 ml) and stirred for 18 h in an ice bath, whereupon a slow flow of ammonia was charged into the solution. The volatiles were removed in vacuo. The residue was washed with diethyl ether and dried to afford 2-(3-nitrophenyl)acetamide (14 g, off-white solid). ¹H NMR (CDCl₃): δ 8.1 (s, 1H), 8.0 (d, 1H), 7.7 (d, 1H), 7.5 (m, 1H), 7.1 (bd s, 1H), 6.2 (brs, 1H), 3.6 (s, 2H).

The crude material from the previous reaction (8 g) and 10% Pd on activated carbon (1 g) in ethanol (100 ml) was hydrogenated at 30 psi for 18 h and filtered over Celite. Removal of the volatiles in vacuo provided 2-(3-aminophenyl)acetamide (5.7 g). A solution of this material (7 g, 46.7 mmol) was dissolved in 6 N HCl (100 ml), cooled to 0° C., and vigorously stirred. Sodium nitrite (3.22 g, 46.7 mmol) in water (50 ml) was added. After 30 min, tin (II) chloride dihydrate (26 g) in 6 N HCl (100 ml) was added. The reaction mixture was stirred at 0° C. for 3 h. The pH was adjusted to pH 14 with 50% aqueous NaOH solution and extracted with ethyl acetate. The combined organic extracts were concentrated in vacuo provided 2-(3-hydrazinophenyl)acetamide.

The crude material from the previous reaction (ca. 15 mmol) and 4,4-dimethyl-3-oxopentanenitrile (1.85 g, 15 mmol) in ethanol (60 ml) and 6 N HCl (1.5 ml) was refluxed for 1 h and cooled to RT. The reaction mixture was neutralized by adding solid sodium hydrogen carbonate. The slurry was filtered and removal of the volatiles in vacuo provided a residue, which was extracted with ethyl acetate. The solvent was removed in vacuo to provide 2-[3-(3-tert-butyl-5-amino-1H-pyrazol-1-yl)phenyl]acetamide as a white solid (3.2 g), which was used without further purification.

### Example 25

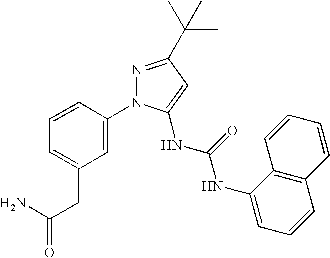

A mixture of Example N (2 g, 0.73 mmol) and 1-naphthylisocyanate (0.124 g, 0.73 mmol) in dry CH₂Cl₂ (4 ml) was stirred at RT under N₂ for 18 h. The solvent was removed in vacuo and the crude product was washed with ethyl acetate (8 ml) and dried in vacuo to yield 1-{3-tert-butyl-1-[3-(carbamoylmethyl)phenyl)-1H-pyrazol-5-yl}-3-(naphthalene-1-yl)urea as a white solid (0.22 g). mp: 230 (dec.); ¹HNMR (200 MHz, DMSO-d₆): δ 9.12 (s, 1H), 8.92 (s, 1H), 8.32-8.08 (m, 3H), 7.94-7.44 (m, 8H), 6.44 (s, 1H), 3.51 (s, 2H), 1.31 (s, 9H); MS:

### Example 26

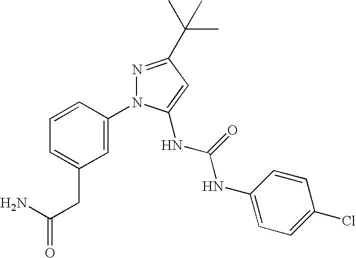

The title compound was synthesized in a manner analogous to Example 23 utilizing Example N (0.2 g, 0.73 mmol) and 4-chlorophenylisocyanate (0.112 g, 0.73 mmol) to yield 1-{3-tert-butyl-1-[3-(carbamoylmethyl)phenyl)-1H-pyrazol-5-yl}-3-(4-chlorophenyl)urea as a white solid (0.28 g). mp: 222 224. (dec.); ¹H NMR (200 MHz, DMSO-d₆); δ 9.15 (s, 1H), 8.46 (s, 1H), 7.55-7.31 (m, 8H), 6.39 (s, 1H), 3.48 (s, 2H), 1.30 (s, 9H); MS:

### Example O

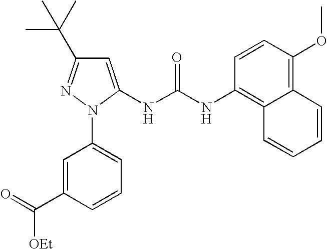

The title compound is synthesized in a manner analogous to Example C utilizing Example A and 1-isocyanato-4-methoxy-naphthalene to yield ethyl 3-(3-tert-butyl-5-(3-(1-methoxynaphthalen-4-yl)ureido)-1H-pyrazol-1-yl)benzoate.

### Example 27

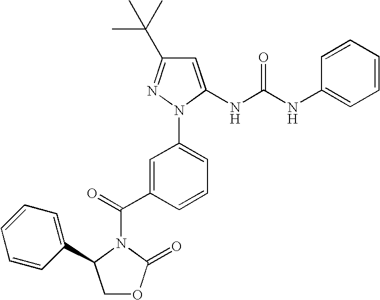

The title compound is synthesized in a manner analogous to Example 17 utilizing Example M and D-4-phenyl-oxazolidin-2-one to yield D-1-{5-tert-butyl-2-[3-(2-oxo-4-phenyl-oxazolidinyl-3-carbonyl)phenyl]-2H-pyrazol-3-yl}-3-phenylurea.

### Example 28

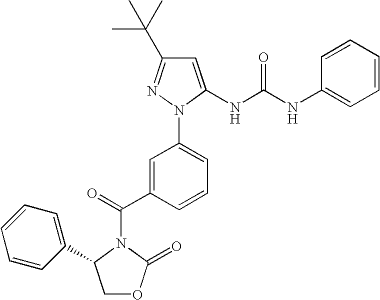

The title compound is synthesized in a manner analogous to Example 17 utilizing Example M and and L-4-phenyl-oxazolidin-2-one to yield L-1-{5-tert-butyl-2-[3-(2-oxo-4-phenyl-oxazolidinyl-3-carbonyl)phenyl]-2H-pyrazol-3-yl}-3-phenylurea.

### Example P

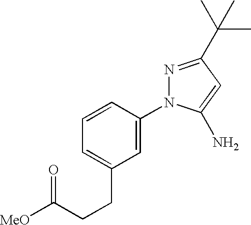

A mixture of 3-(3-amino-phenyl)-acrylic acid methyl ester (6 g) and 10% Pd on activated carbon (1 g) in ethanol (50 ml) was hydrogenated at 30 psi for 18 h and filtered over Celite. Removal of the volatiles in vacuo provided 3-(3-amino-phenyl)propionic acid methyl ester (6 g).

A vigorously stirred solution of the crude material from the previous reaction (5.7 g, 31.8 mmol) dissolved in 6 N HCl (35 ml) was cooled to 0° C., and sodium nitrite (2.2 g) in water (20 ml) was added. After 1 h, tin (II) chloride dihydrate (18 g) in 6 N HCl (35 ml) was added. And the mixture was stirred at 0° C. for 3 h. The pH was adjusted to pH 14 with solid KOH and extracted with EtOAc. The combined organic extracts were concentrated in vacuo provided methyl 3-(3-hydrazino-phenyl)propionate (1.7 g).

A stirred solution of the crude material from the previous reaction (1.7 g, 8.8 mmol) and 4,4-dimethyl-3-oxopentanenitrile (1.2 g, 9.7 mmol) in ethanol (30 ml) and 6 N HCl (2 ml) was refluxed for 18 h and cooled to RT. The volatiles were removed in vacuo and the residue dissolved in EtOAc and washed with 1 N aqueous NaOH. The organic layer was dried (Na₂SO₄) and concentrated in vacuo and the residue was purified by column chromatography using 30% ethyl acetate in hexane as the eluent to provide methyl 3-[3-(3-tert-butyl-5-amino-1H-pyrazol-1-yl)phenyl]propionate (3.2 g), which was used without further purification

### Example 29

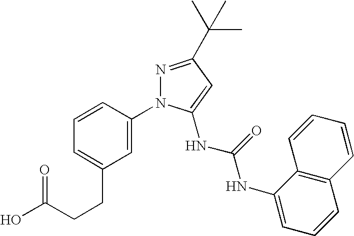

A mixture of Example P (0.35 g, 1.1 mmol) and 1-naphthylisocyanate (0.19 g, 1.05 mmol) in dry CH₂Cl₂ (5 ml) was stirred at RT under N₂ for 20 h. The solvent was removed in vacuo and the residue was stirred in a solution of THF (3 ml)/MeOH (2 ml)/water (1.5 ml) containing lithium hydroxide (0.1 g) for 3 h at RT, and subsequently diluted with EtOAc and dilute citric acid solution. The organic layer was dried (Na₂SO₄), and the volatiles removed in vacuo. The residue was purified by column chromatography using 3% methanol in CH₂Cl₂ as the eluent to yield 3-(3-{3-tert-butyl-5-[3-(naphthalen-1-yl)ureido]-1H-pyrazol-1-yl)phenylpropionic acid (0.22 g, brownish solid). mp: 105-107; ¹H NMR (200 MHz, CDCl₃): δ 7.87-7.36 (m, 10H), 7.18-7.16 (m, 1H), 6.52 (s, 1H), 2.93 (t, J=6.9 Hz, 2H), 2.65 (t, J=7.1 Hz, 2H), 1.37 (s, 9H); MS

### Example 30

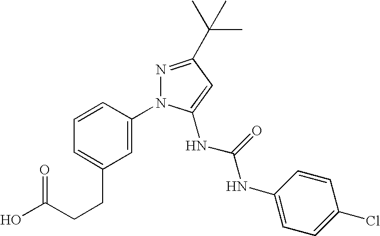

The title compound was synthesized in a manner analogous to Example 29 utilizing Example P (0.30 g, 0.95 mmol) and 4-chlorophenylisocyanate (0.146 g, 0.95 mmol) to yield 3-(3-{3-tert-butyl-5-[3-(4-chlorophenyl)ureido]-1H-pyrazol-1-yl)phenyl)propionic acid (0.05 g, white solid). mp: 85 87; ¹HNMR (200 MHz, CDCl₃): δ 8.21 (s, 1H), 7.44-7.14 (m, 7H), 6.98 (s, 1H), 6.55 (s, 1H), 2.98 (t, J=5.2 Hz, 2H), 2.66 (t, J=5.6 Hz, 2H), 1.40 (s, 9H); MS

### Example Q

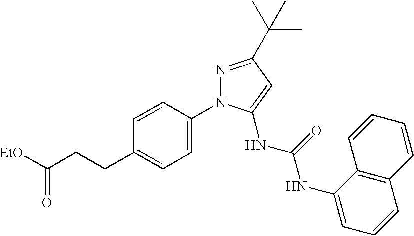

A mixture of ethyl 3-(4-aminophenyl)acrylate (1.5 g) and 10% Pd on activated carbon (0.3 g) in ethanol (20 ml) was hydrogenated at 30 psi for 18 h and filtered over Celite. Removal of the volatiles in vacuo provided ethyl 3-(4-aminophenyl)propionate (1.5 g).

A solution of the crude material from the previous reaction (1.5 g, 8.4 mmol) was dissolved in 6 N HCl (9 ml), cooled to 0° C., and vigorously stirred. Sodium nitrite (0.58 g) in water (7 ml) was added. After 1 h, tin (II) chloride dihydrate (5 g) in 6 N HCl (10 ml) was added. The reaction mixture was stirred at 0° C. for 3 h. The pH was adjusted to pH 14 with solid KOH and extracted with EtOAc. The combined organic extracts were concentrated in vacuo provided ethyl 3-(4-hydrazino-phenyl)-propionate (1 g).

The crude material from the previous reaction (1 g, 8.8 mmol) and 4,4-dimethyl-3-oxopentanenitrile (0.7 g) in ethanol (8 ml) and 6 N HCl (1 ml) was refluxed for 18 h and cooled to RT. The volatiles were removed in vacuo. The residue was dissolved in ethyl acetate and washed with 1 N aqueous sodium hydroxide solution. The organic layer was dried (Na₂SO₄) and concentrated in vacuo. The residue was purified by column chromatography using 0.7% methanol in CH₂Cl₂ as the eluent to provide ethyl 3-{4-[3-tert-butyl-5-(3-(naphthalene-1-yl)ureido]-1H-pyrazol-1-yl}phenyl)propanoate (0.57 g).

### Example 31

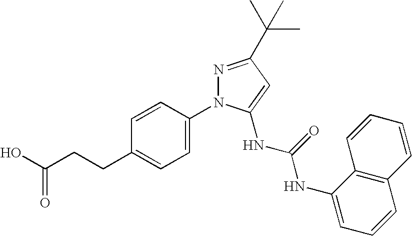

A mixture of Example Q (0.25 g, 0.8 mmol) and 1-naphthylisocyanate (0.13 g, 0.8 mmol) in dry CH₂Cl₂ (5 ml) was stirred at RT under N₂ for 20 h. The solvent was removed in vacuo and the residue was stirred in a solution of THF (3 ml)/MeOH (2 ml)/water (1.5 ml) containing lithium hydroxide (0.1 g) for 3 h at RT and diluted with EtOAc and diluted citric acid solution. The organic layer was dried (Na₂SO₄), and the volatiles removed in vacuo. The residue was purified by column chromatography using 4% methanol in CH₂Cl₂ as the eluent to yield 3-{4-[3-tert-butyl-5-(3-(naphthalene-1-yl)ureido]-1H-pyrazol-1-yl}phenyl)propanonic acid (0.18 g, off-white solid). mp: 120 122; ¹H NMR (200 MHz, CDCl₃): δ 7.89-7.06 (m, 11H), 6.5 (s, 1H), 2.89 (m, 2H), 2.61 (m, 2H), 1.37 (s, 9H); MS

### Example 32

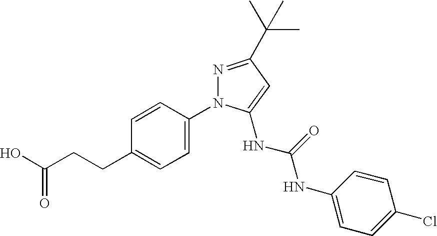

The title compound was synthesized in a manner analogous to Example 31 utilizing Example Q (0.16 g, 0.5 mmol) and 4-chlorophenylisocyanate (0.077 g, 0.5 mmol) to yield 3-{4-[3-tert-butyl-5-(3-(4-chlorophenyl)ureido]-1H-pyrazol-1-yl}phenyl)propanonic acid acid (0.16 g, off-white solid). mp: 112-114; ¹H NMR (200 MHz, CDCl₃): δ 8.16 (s, 1H), 7.56 (s, 1H), 7.21 (s, 2H), 7.09 (s, 2H), 6.42 (s, 1H), 2.80 (m, 2H), 2.56 (m, 2H), 1.32 (s, 9H); MS

### Example R

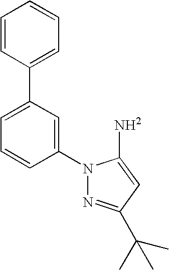

A 250 mL pressure vessel (ACE Glass Teflon screw cap) was charged with 3-nitrobiphenyl (20 g, 0.10 mol) dissolved in THF (˜100 mL) and 10% Pd/C (3 g). The reaction vessel was charged with H₂ (g) and purged three times. The reaction was charged with 40 psi H₂ (g) and placed on a Parr shaker hydrogenation apparatus and allowed to shake overnight at RT. HPLC showed that the reaction was complete thus the reaction mixture was filtered through a bed of Celite and evaporated to yield the amine: 16.7 g (98% yield)

In a 250 mL Erlenmeyer flask with a magnetic stir bar, the crude material from the previous reaction (4.40 g, 0.026 mol) was added to 6 N HCl (40 mL) and cooled with an ice bath to 0° C. A solution of NaNO₂ (2.11 g, 0.0306 mol, 1.18 eq.) in water (5 mL) was added drop wise. After 30 min, SnCl₂.2H₂O (52.0 g, 0.23 mol, 8.86 eq.) in 6N HCl (100 mL) was added and the reaction mixture was allowed to stir for 3 h, then subsequently transferred to a 500 mL round bottom flask. To this, 4,4-dimethyl-3-oxopentanenitrile (3.25 g, 0.026 mol) and EtOH (100 ml) were added and the mixture refluxed for 4 h, concentrated in vacuo and the residue extracted with EtOAc (2×100 mL). The residue was purified by column chromatograph using hexane/EtOAc/Et₃N (8:2:0.2) to yield 0.53 g of Example R. ¹H NMR (CDCl₃): δ 7.5 (m, 18H), 5.8 (s, 1H), 1.3 (s, 9H).

### Example 33

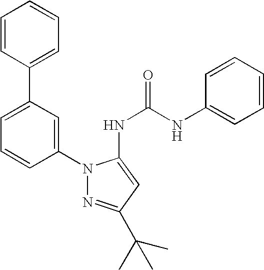

In a dry vial with a magnetic stir bar, Example R (0.145 g; 0.50 mmol) was dissolved in 2 mL CH₂Cl₂ (anhydrous) followed by the addition of phenylisocyanate (0.0544 mL; 0.50 mmol; 1 eq.). The reaction was kept under argon and stirred for 17 h. Evaporation of solvent gave a crystalline mass that was triturated with hexane/EtOAc (4:1) and filtered to yield 1-(3-tert-butyl-1-(3-phenylphenyl)-1H-pyrazol-5-yl)-3-phenylurea (0.185 g, 90%). HPLC purity: 96%; mp: 80 84; ¹H NMR (CDCl₃): δ7.3 (m, 16H), 6.3 (s, 1H), 1.4 (s, 9H).

### Example 34

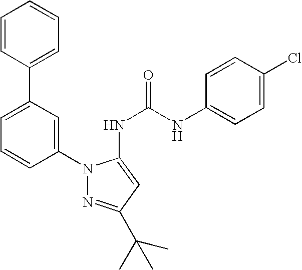

The title compound was synthesized in a manner analogous to Example 33 utilizing Example R (0.145 g; 0.50 mmol) and p-chlorophenylisocyanate (0.0768 g, 0.50 mmol, 1 eq.) to yield 1-(3-tert-butyl-1-(3-phenylphenyl)-1H-pyrazol-5-yl)-3-(4-chlorophenyl)urea (0.205 g, 92%). HPLC purity: 96.5%; mp: 134 136; ¹H NMR (CDCl₃): δ 7.5 (m, 14H), 7.0 (s, 1H), 6.6 (s, 1H), 6.4 (s, 1H), 1.4 (s, 9H).

### Example S

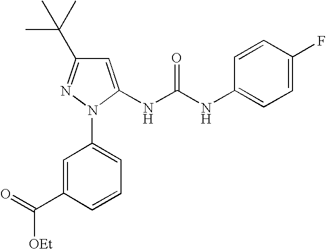

The title compound is synthesized in a manner analogous to Example C utilizing Example A and 4-fluorophenyl isocyanate yield ethyl 3-(3-tert-butyl-5-(3-(4-fluorophenyl)ureido)-1H-pyrazol-1-yl)benzoate.

### Example 35

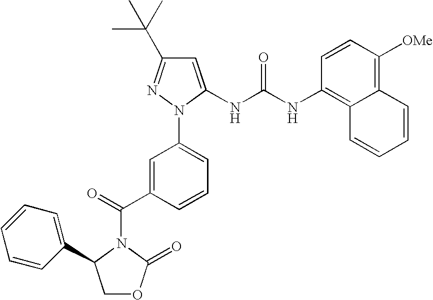

The title compound is synthesized in a manner analogous to Example 17 utilizing Example M and D-4-phenyl-oxazolidin-2-one to yield D-1-{5-tert-butyl-2-[3-(2-oxo-4-phenyl-oxazolidinyl-3-carbonyl)phenyl]-2H-pyrazol-3-yl}-3-(naphthalen-1-yl)urea.

### Example 36

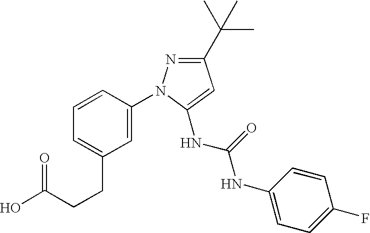

The title compound is synthesized in a manner analogous to Example 29 utilizing Example P (0.30 g, 0.95 mmol) and 4-flu0rophenylisocyanate (0.146 g, 0.95 mmol) to yield 3-(3-(3-tert-butyl-5-(3-(4-fluorophenyl)ureido)-1H-pyrazol-1-yl)phenyl)propanoic acid.

### Example T

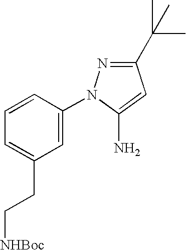

To a stirred solution of Example N (2 g, 7.35 mmol) in THF (6 ml) was added borane-methylsulfide (18 mmol). The mixture was heated to reflux for 90 min and cooled to RT, after which 6 N HCl was added and heated to reflux for 10 min. The mixture was basified with NaOH and extracted with EtOAc. The organic layer was dried (Na₂SO₄) filtered and concentrated in vacuo to yield 3-tert-butyl-1-[3-(2-aminoethyl)phenyl]-1H-pyrazol-5 amine (0.9 g).

A mixture of the crude material from the previous reaction (0.8 g, 3.1 mmol) and di-tert-butylcarbonate (0.7 g, 3.5 mmol) and catalytically amount of DMAP in dry CH₂Cl₂ (5 ml) was stirred at RT under N₂ for 18 h. The reaction mixture was concentrated in vacuo and the residue was purified by column chromatography using 1% methanol in CH₂Cl₂ as the eluent to yield tert-butyl 3-(3-tert-butyl-5-amino-1H-pyrazol-1-yl)phenylcarbamate (0.5 g).

### Example 37

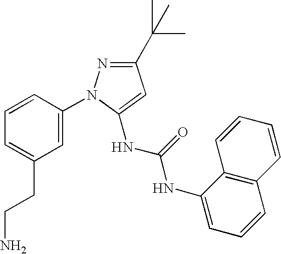

A mixture of Example T (0.26 g, 0.73 mmol) and 1-naphthylisocyanate (0.123 g, 0.73 mmol) in dry CH₂Cl₂ (5 ml) was stirred at RT under N₂ for 48 h. The solvent was removed in vacuo and the residue was purified by column chromatography using 1% methanol in CH₂Cl₂ as the eluent (0.15 g, off-white solid). The solid was then treated with TFA (0.2 ml) for 5 min and diluted with EtOAc. The organic layer was washed with saturated NaHCO₃ solution and brine, dried (Na₂SO₄), filtered and concentrated in vacuo to yield 1-{3-tert-butyl-1-[3-(2-Aminoethyl)phenyl]-1H-pyrazol-5-yl}-3-(naphthalen-1-yl)ureas a solid (80 mg). mp: 110-112; ¹H NMR (200 MHz, DMSO-d₆): δ 9.09 (s, 1H), 8.90 (s, 1H), 8.01-7.34 (m, 11H), 6.43 (s, 1H), 3.11 (m, 2H), 2.96 (m, 2H), 1.29 (s, 9H); MS

### Example 38

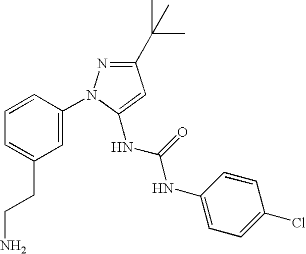

The title compound was synthesized in a manner analogous to Example 37 utilizing Example T (0.15 g, 0.42 mmol) and 4-chlorophenylisocyanate (0.065 g, 0.42 mmol) to yield 1-(3-tert-butyl-1-[3-(2-Aminoethyl)phenyl]-1H-pyrazol-5-yl}-3-(4-chlorophenyl)urea as an off-white solid (20 mg). mp:125-127; ¹H NMR (200 MHz, CDCl₃): δ 8.81 (s, 1H), 8.66 (s, 1H), 7.36-7.13 (m, 8H), 6.54 (s, 1H), 3.15 (brs, 2H), 2.97 (brs, 2H), 1.32 (s, 9H); MS

### Example U

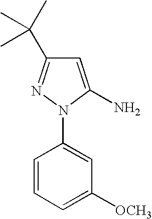

In a 250 mL Erlenmeyer flask with a magnetic stir bar, m-anisidine (9.84 g, 0.052 mol) was added to 6 N HCl (80 mL) and cooled with an ice bath to 0° C. A solution of NaNO₂ (4.22 g, 0.0612 mol, 1.18 eq.) in water (10 mL) was added drop wise. After 30 min, SnCl₂.2H₂O (104.0 g, 0.46 mol, 8.86 eq.) in 6 N HCl (200 mL) was added and the reaction mixture was allowed to stir for 3 h., and then subsequently transferred to a 1000 mL round bottom flask. To this, 4,4-dimethyl-3-oxopentanenitrile (8.00 g, 0.064 mol) and EtOH (200 mL) were added and the mixture refluxed for 4 h, concentrated in vacuo and the residue recrystallized from CH₂Cl₂ to yield 3-tert-butyl-1-(3-methoxyphenyl)-1H-pyrazol-5-amine as the HCl salt (13.9 g).

The crude material from the previous reaction (4.65 g, 0.165 mol) was dissolved in 30 mL of CH₂Cl₂ with Et₃N (2.30 mL, 0.0165 mol, 1 eq.) and stirred for 30 min Extraction with water followed by drying of the organic phase with Na₂SO₄ and concentration in vacuo yielded a brown syrup that was the free base, 3-tert-butyl-1-(3-methoxyphenyl)-1H-pyrazol-5-amine (3.82 g, 94.5%), which was used without further purification.

### Example 39

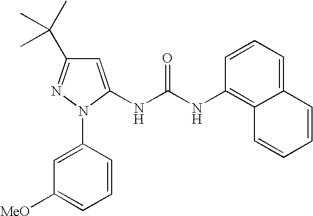

In a dry vial with a magnetic stir bar, Example U (2.62 g, 0.0107 mol) was dissolved in CH₂Cl₂ (5 mL, anhydrous) followed by the addition of 1-naphthylisocyanate (1.53 mL, 0.0107 mol, 1 eq.). The reaction was kept under Ar and stirred for 18 h. Evaporation of solvent followed by column chromatography with EtOAc/hexane/Et₃N (7:2:0.5) as the eluent yielded 1-[3-tert-butyl-1-(3-methoxyphenyl)-1H-pyrazol-5-yl]-3-(naphthalen-1-yl)urea (3.4 g, 77%). HPLC: 97%; mp: 78-80; ¹H NMR (CDCl₃): δ 7.9-6.8 (m, 15H), 6.4 (s, 1H), 3.7 (s, 3H), 1.4 (s, 9H).

### Example 40

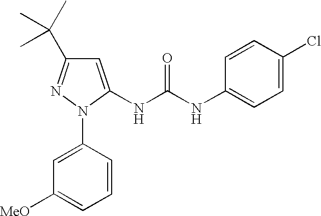

The title compound was synthesized in a manner analogous to Example 39 utilizing Example U (3.82 g; 0.0156 mol) and p-chlorophenylisocyanate (2.39 g, 0.0156 mol, 1 eq.), purified by trituration with hexane/EtOAc (4:1) and filtered to yield 1-[3-tert-butyl-1-(3-methoxyphenyl)-1H-pyrazol-5-yl]-3-(4-chlorophenyl)urea (6.1 g, 98%). HPLC purity: 95%; mp: 158-160,H NMR (CDCl₃): δ 7.7 (s, 1H); δ 7.2 6.8 (m, 8H), 6.4 (s, 1H), 3.7 (s; 3H), 1.3 (s, 9H).

### Example 41

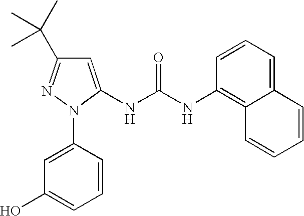

In a 100 ml round bottom flask equipped with a magnetic stir bar, Example 39 (2.07 g) was dissolved in CH₂Cl₂ (20 mL) and cooled to 0° C. with an ice bath. BBr₃ (1 M in CH₂Cl₂; 7.5 mL) was added slowly. The reaction mixture was allowed to warm warm to RT overnight. Additional BBr₃ (1 M in CH₂Cl₂, 2×1 mL, 9.5 mmol total added) was added and the reaction was quenched by the addition of MeOH. Evaporation of solvent led to a crystalline material that was chromatographed on silica gel (30 g) using CH₂Cl₂/MeOH (9.6:0.4) as the eluent to yield 1-[3-tert-butyl-1-(3-hydroxyphenyl)-1H-pyrazol-5-yl]-3-(naphthalene-1-yl)urea (0.40 g, 20%). ¹HNMR (DMSO-d₆): δ 9.0 (s, 1H), 8.8 (s, 1H), 8.1-6.8 (m, 11H), 6.4 (s, 1H), 1.3 (s, 9H). MS (ESI) m/z: 401 (M+H⁺).

### Example 42

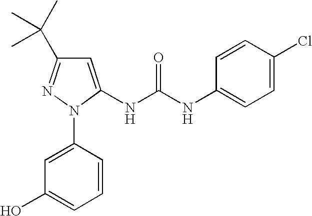

The title compound was synthesized in a manner analogous to Example 41 utilizing Example 40 (2.00 g, 5 mmol) that resulted in a crystalline material that was filtered and washed with MeOH to yield 1-[3-tert-butyl-1-(3-hydroxyphenyl)-1H-pyrazol-5-yl]-3-(4-chlorophenyl)urea (1.14 g, 60%). HPLC purity: 96%; mp: 214-216; ¹H NMR (CDCl₃): δ 8.4 (s, 1H), 7.7 (s, 1H), 7.4-6.6 (m, 9H), 1.3 (s, 9H).

### Example V

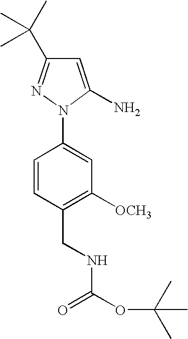

The starting material, 1-[4-(aminomethyl)phenyl]-3-tert-butyl-N-nitroso-1H-pyrazol-5-amine, was synthesized in a manner analogous to Example A utilizing 4-aminobenzamide and 4,4-dimethyl-3-oxopentanenitrile.

A 1 L four-necked round bottom flask was equipped with a stir bar, a source of dry Ar, a heating mantle, and a reflux condenser. The flask was flushed with Ar and charged with the crude material from the previous reaction (12 g, 46.5 mmol; 258.1 g/mol) and anhydrous THF (500 ml). This solution was treated cautiously with LiAlH₄ (2.65 g, 69.8 mmol) and the reaction was stirred overnight. The reaction was heated to reflux and additional LiAlH₄ was added complete (a total of 8.35 g added). The reaction was cooled to 0 and H₂O (8.4 ml), 15% NaOH (8.4 ml) and H₂O (24 ml) were added sequentially; The mixture was stirred for 2 h, the solids filtered through Celite, and washed extensively with THF, the solution was concentrated in vacuo to yield 1-(4-(aminomethyl-3-methoxy)phenyl)-3-tert-butyl-1H-pyrazol-5-amine (6.8 g) as an oil.

A 40 mL vial was equipped with a stir bar, a septum, and a source of Ar. The vial was charged with the crude material from the previous reaction (2 g, 8.2 mmol, 244.17 g/mol) and CHCl₃ (15 mL) were cooled to 0 under Ar and di-tert-butylcarbonate (1.9 g, 9.0 mmol) dissolved in CHCl₃ (5 mL) was added drop wise over a 2 min period. The mixture was treated with 1N KOH (2 mL), added over a 2 h period. The resulting emulsion was broken with the addition of saturated NaCl solution, the layers were separated and the aqueous phase extracted with CH₂Cl₂ (2×1.5 ml). The combined organic phases were dried over Na₂SO₄, filtered, concentrated in vacuo to yield tert-butyl[4-(3-tert-butyl-5-amino-1H-pyrazol-1-yl)-2-methoxybenzylcarbamate (2.23 g, 79%) as a light yellow solid. ¹H NMR (CDCl₃): δ 7.4 (m, 5H), 5.6 (s, 1H), 4.4 (d, 2H), 1.5 (s, 9H), 1.3 (s, 9H).

### Example 43

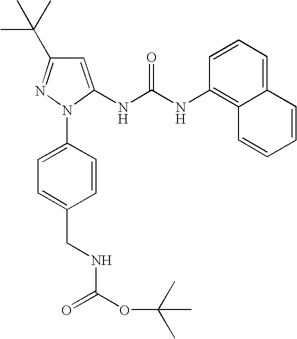

A 40 mL vial was equipped with a septum, a stir bar and a source of Ar, and charged with Example V (2 g, 5.81 mmol), flushed with Ar and dissolved in CHCl₃ (20 mL). The solution was treated with 2-naphthylisocyanate (984 mg, 5.81 mmol) in CHCl₃ (5 mL) and added over 1 min The reaction was stirred for 8 h, and additional 1-naphthylisocyanate (81 mg) was added and the reaction stirred overnight. The solid was filtered and washed with CH₂Cl₂ to yield tert-butyl 4-[3-tert-butyl-5-(3-naphthalen-1-yl)ureido)-1H-pyrazol-1-yl]benzylcarbamate (1.2 g). HPLC purity: 94.4%; ¹H NMR (DMSO-d₆): δ 9.1 (s, 1H), 8.8 (s, 1H), 8.0 (m, 3H), 7.6 (m, 9H), 6.4 (s, 1H), 4.2 (d, 2H), 1.4 (s, 9H), 1.3 (s, 9H).

### Example 44

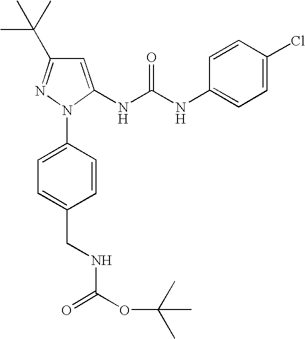

The title compound was synthesized in a manner analogous to Example 43 utilizing Example V (2.0 g, 5.81 mmol) and p-chlorophenylisocyanate (892 mg) to yield tert-butyl 4-[3-tert-butyl-5-(3-(4-chlorophenyl)ureido)-1H-pyrazol-1-yl]benzylcarbamate (1.5 g). HPLC purity: 97%; ¹H NMR (DMSO-d₆): δ 9.2 (s, 1H), 8.4 (s, 1H), 7.4 (m, 8H), 6.4 (s, 1H), 4.2 (d, 2H), 1.4 (s, 9H), 1.3 (s, 9H).

### Example 45

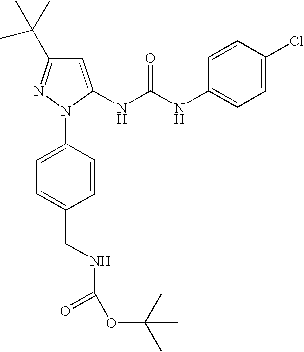

A 10 mL flask equipped with a stir bar was flushed with Ar and charged with Example 43 (770 mg, 1.5 mmol) and CH₂Cl₂ (1 ml) and 1:1 CH₂Cl₂:TFA (2.5 mL). After 1.5 h, reaction mixture was concentrated in vacuo, the residue was dissolved in EtOAc (15 mL), washed with saturated NaHCO₃ (10 mL) and saturated NaCl (10 mL). The organic layers was dried, filtered and concentrated in vacuo to yield 1-{3-tert-butyl-1-[4-(aminomethyl)phenyl]-1H-pyrazol-5-yl}-3-(naphthalen-1-yl)urea (710 mg). ¹HNMR (DMSO-d₆): δ 7.4 (m, 11H), 6.4 (s, 1H), 3.7 (s, 2H), 1.3 (s, 9H).

### Example 46

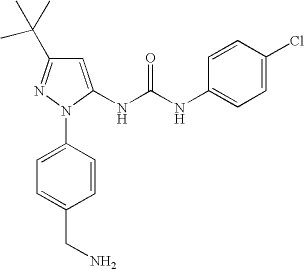

The title compound was synthesized in a manner analogous to Example 45 utilizing Example 44 (1.5 g, 1.5 mmol) to yield 1-{3-tert-butyl-1-[4-(aminomethyl)phenyl]-1H-pyrazol-5-yl}-3-(4-chlorophenyl)urea (1.0 g). HPLC purity: 93.6%; mp: 100-102; ¹H NMR (CDCl₃): δ 8.6 (s, 1H), 7.3 (m, 8H), 6.3 (s, 1H), 3.7 (brs, 2H), 1.3 (s, 9H).

### Example 47

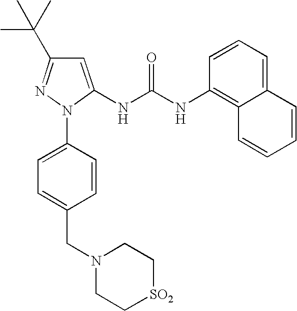

A 10 ml vial was charged with Example 45 (260 mg, 63 mmol) and absolute EtOH (3 mL) under Ar. Divinylsulfone (63 uL, 74 mg, 0.63 mmol) was added drop wise over 3 min and the reaction was stirred at RT for 1.5 h. and concentrated in vacuo to yield a yellow solid, which was purified via preparative TLC, developed in 5% MeOH:CH₂Cl₂. The predominant band was cut and eluted off the silica with 1:1 EtOAc:MeOH, filtered and concentrated in vacuo to yield 1-{3-tert-butyl-1-[4-(1,1-dioxothiomorpholin-4-yl)methylphenyl]-1H-pyrazol-5-yl}-3-(naphthalen-1-yl)urea (150 mg). HPLC purity: 96%; ¹H NMR (DMSO-d₆): δ 9.1 (s, 1H), 9.0 (s, 1H), 7.9 (m, 3H), 7.5 (m, 8H), 6.4 (s, 1H), 3.1 (brs, 4H), 2.9 (brs, 4H), 1.3 (s, 9H).

### Example 48

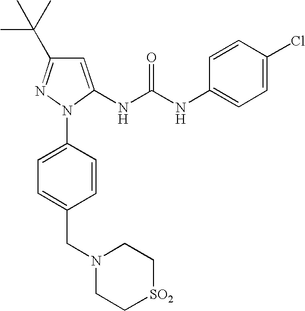

The title compound was synthesized in a manner analogous to Example 47 utilizing Example 46 (260 mg, 0.66 mmol) to yield 1-{3-tert-butyl-1-[4-(1,1-dioxothiomorpholin-4-yl)methylphenyl]-1H-pyrazol-5-yl}-3-(4-chlorophenyl)urea (180 mg). HPLC purity: 93%; mp: 136-138; ¹H NMR (DMSO-d₆): δ 9.2 (s, 1H), 8.5 (s, 1H), 7.4 (m, 9H), 6.4 (s, 1H), 3.1 (brs, 4H), 3.0 (brs, 4H), 1.3 (s, 9H).

### Example 49

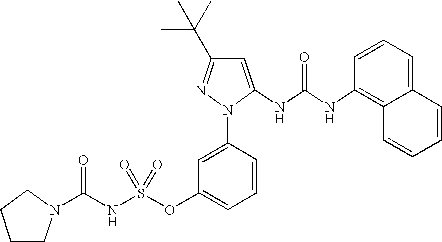

To a stirring solution of chlorosulfonyl isocyanate (0.35 g, 5 mmol) in CH₂Cl₂ (20 mL) at 0° C. was added pyrrolidine (0.18 g, 5 mmol) at such a rate that the reaction temperature did not rise above 5° C. After stirring for 2 h, a solution of Example 41 (1.10 g, 6.5 mmol) and triethylamine (0.46 g, 9 mmol) in CH₂Cl₂ (20 mL) was added. When the addition was complete, the mixture was allowed to warm to RT and stirred overnight. The reaction mixture was poured into 10% HCl (10 mL) saturated with NaCl, the organic layer was separated and the aqueous layer extracted with ether (20 mL). The combined organic layers were dried (Na₂SO₄) and concentrated in vacuo, purified by preparative HPLC to yield (pyrrolidine-1-carbonyl)sulfamic acid 3-[3-tert-butyl-5-(3-naphthalen-1-yl-ureido)-pyrazol-1-yl]phenyl ester (40 mg). ¹H NMR (CDCl₃): δ 9.12 (brs, 1H), 8.61 (brs, 1H), 7.85-7.80 (m, 3H), 7.65 (d, J=8.0 Hz, 2H), 7.53-7.51 (m, 1H), 7.45-7.25 (m, 5H), 6.89 (s, 4H), 3.36-3.34 (brs, 1H), 3.14-3.13 (brs, 2H), 1.69 (brs, 2H), 1.62 (brs, 2H), 1.39 (s, 9H); MS (ESI) m/z: 577 (M+H⁺).

### Example 50

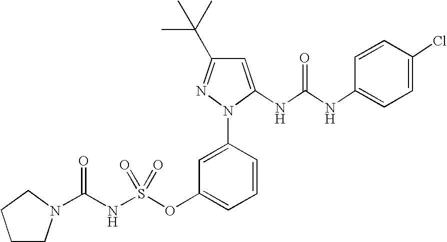

The title compound was synthesized in a manner analogous to Example 49 utilizing Example 42 to yield (pyrrolidine-1-carbonyl)sulfamic acid 3-[3-tert-butyl-5-(4-chlorophenyl-1-yl-ureido)pyrazol-1-yl]phenyl ester. MS (ESI) m/z: 561 (M+H⁺).

### Example W

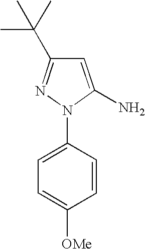

Solid 4-methoxyphenylhydrazine hydrochloride (25.3 g) was suspended in toluene (100 mL) and treated with triethylamine (20.2 g). The mixture was stirred at RT for 30 min and treated with pivaloylacetonitrile (18 g). The reaction was heated to reflux and stirred overnight. The hot mixture was filtered, the solids washed with hexane and dried in vacuo to afford 3-tert-butyl-1-(4-methoxyphenyl)-1H-pyrazol-5-amine (25 g, 70%). ¹H NMR (DMSO-d₆): δ 7.5 (d, 2H), 7.0 (d, 1H), 6.4 (s, 1H), 6.1 (s, 2H), 3.9 (s, 3H), 1.3 (s, 9H).

### Example 51

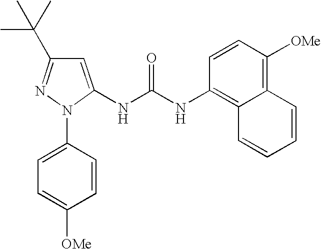

To a solution of 1-isocyanato-4-methoxy-naphthalene (996 mg) in anhydrous CH₂Cl₂ (20 mL) of was added Example W (1.23 g). The reaction solution was stirred for 3 h, the resulting white precipitate filtered, treated with 10% HCl and recrystallized from MeOH, and dried in vacuo to yield 1-[3-tert-butyl-1-(4-methoxyphenyl)-1H-pyrazol-5-yl]-3-(1-methoxynaphthalen-4-yl-urea as white crystals (900 mg, 40%). HPLC purity: 96%; mp: 143-144; ¹H NMR (DMSO-d₆): δ 8.8 (s, 1H), 8.5 (s, 1H), 8.2 (d, 1H), 8.0 (d, 1H), 7.6 (m, 5H), 7.1 (d, 2H), 7.0(d, 1H), 6.3 (s, 1H), 4.0 (s, 3H), 3.9 (s, 3H); 1.3 (s, 9H).

### Example 52

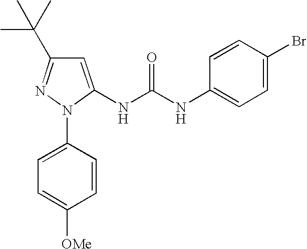

The title compound was synthesized in a manner analogous to Example 51 utilizing Example W and p-bromophenylisocyanate (990 mg) to yield 1-{3-tert-butyl-1-(4-methoxyphenyl)-1H-pyrazol-5-yl}-3-(4-bromophenyl)urea as off-white crystals (1.5 g, 68%). HPLC purity: 98%; mp: 200-201; ¹HNMR (DMSO-d₆): δ 9.3 (s, 1H), 8.3 (s, 1H), 7.4 (m, 6H), 7.0 (d, 2H), 6.3 (s, 1H), 3.8 (s, 3H), 1.3 (s, 9H).

### Example 53

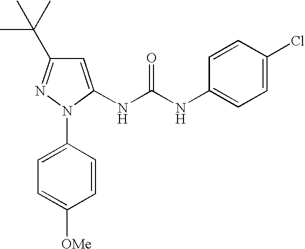

The title compound was synthesized in a manner analogous to Example 51 utilizing Example W and p-chlorophenylisocyanate (768 mg) into yield 1-{3-tert-butyl-1-(4-methoxyphenyl)-1H-pyrazol-5-yl}-3-(4-chlorophenyl)ureas white crystals (10.3 g, 65%). HPLC purity: 98%; mp: 209-210; 1H NMR (DMSO-d₆): δ 9.1 (s, 1H), 8.3 (s, 1H), 7.4 (m, 4H), 7.3 (d, 2H), 7.1 (d, 2H), 6.3 (s, 1H), 3.8 (s, 3H), 1.3 (s, 9H).

### Example 54

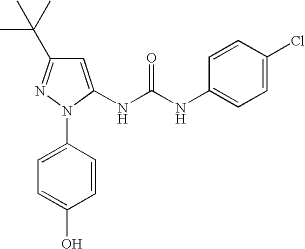

The title compound was synthesized in a manner analogous to Example 41 utilizing Example 53 (500 mg) to yield 1-{3-tert-butyl-1-(4-hydroxyphenyl)-1H-pyrazol-5-yl}-3-(4-chlorophenyl)urea as white crystals (300 mg, 62%). HPLC purity: 94%; mp: 144-145; ¹H NMR (DMSO-d₆): δ 9.7 (s, 1H), 9.1 (s, 1H), 8.3 (s, 1H), 7.4 (d, 2H), 7.3 (m, 4H); 6.9 (d, 2H), 6.3 (s, 1H), 1.3 (s, 9H)

### Example 55

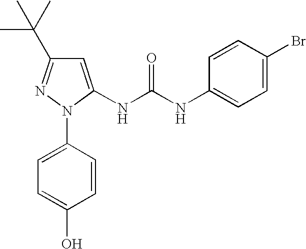 The title compound was synthesized in a manner analogous to Example 41 utilizing Example 52 (550 mg) to yield 1-{3-tert-butyl-1-(4-hydroxyphenyl)-1H-pyrazol-5-yl}-3-(4-bromophenyl)urea as a white crystalline solid (400 mg, 70%). HPLC purity: 93%; mp: 198 200; ¹H NMR (DMSO-d₆): δ 9.7 (s, 1H), 9.2 (s, 1H), 8.3 (s, 1H), 7.4 (d, 4H), 7.2 (m, 2H), 6.9 (d, 2H), 6.3 (s, 1H), 1.3 (s, 9H).

### Example X

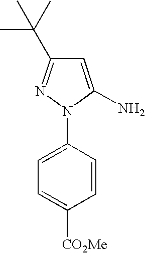

Methyl 4-(3-tert-butyl-5-amino-1H-pyrazol-1-yl)benzoate (3.67 mmol) was prepared from methyl 4-hydrazinobenzoate and pivaloylacetonitrile by the procedure of Regan, et al., J. Med. Chem., 45, 2994 (2002).

### Example 56

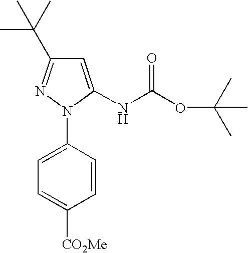

A 500 mL round bottom flask was equipped with a magnetic stir bar and an ice bath. The flask was charged with Example X (1 g) and this was dissolved in CH₂Cl₂ (100 mL). Saturated sodium bicarbonate (100 mL) was added and the mixture rapidly stirred, cooled in an ice bath and treated with diphosgene (1.45 g) and the heterogeneous mixture stirred for 1 h. The layers were separated and the CH₂Cl₂ layer treated with tert-butanol (1.07 g) and the solution stirred overnight at RT. The solution was washed with H₂O (2×150 mL), dried (Na₂SO₄), filtered, concentrated in vacuo, and purified by flash chromatography using 1:2 ethyl acetate:hexane as the eluent to yield tert-buthyl 1-(4-(methoxycarbonyl)phenyl)-3-tert-butyl-1H-pyrazol-5-ylcarbamate (100 mg) as an off-white solid. ¹H NMR (DMSO-d₆): δ 9.2 (s, 1H), 8.1 (d, 2H), 7.7 (d, 2H), 6.3 (s, 1H), 3.3 (s, 3H), 1.3 (s, 18H).

### Example 57

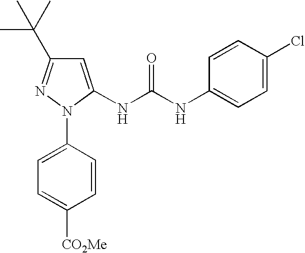

The title compound was synthesized in a manner analogous to Example 41 utilizing Example X (1.37 g) and p-chlorophenylisocyanate (768 mg) to yield methyl 4-{3-tert-butyl-5-[3-(4-chlorophenyl)ureido]-1H-pyrazol-1-yl}benzoate as white crystals (1.4 g 66%). HPLC purity: 98%; mp: 160-161; ¹H NMR (DMSO-d₆): δ 9.2 (s, 1H), 8.6 (s, 1H), 8.1 (d, 2H), 7.8 (d, 2H), 7.5 (d, 2H), 7.3 (d, 2H), 6.4 (s, 1H), 3.9 (s, 3H), 1.3 (s, 9H).

### Example 58

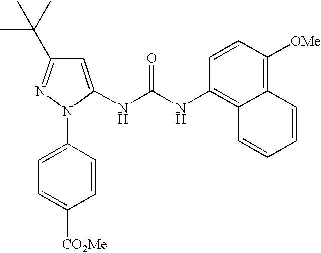

The title compound was synthesized in a manner analogous to Example 41 utilizing Example X (1.27 g) and 1-isocyanato-4-methoxy-naphthalene (996 mg) to yield methyl 4-{3-tert-butyl-5-[3-(1-methoxynaphthalen-4-yl)ureido]-1H-pyrazol-1-yl}benzoate as white crystals (845 mg, 36%). HPLC purity: 98%; mp: 278 280; ¹H NMR (DMSO-d₆): δ 8.76 (s, 1H), 8.73 (s, 1H), 8.1 (m, 3H), 7.9 (d, 1H), 7.7 (d, 2H), 7.6 (m, 3H), 7.0 (d, 1H), 7.0 (d, 1H), 6.3 (s, 1H), 4.0 (s, 3H), 3.9 (s, 3H), 1.3 (s, 9H).

### Example 59

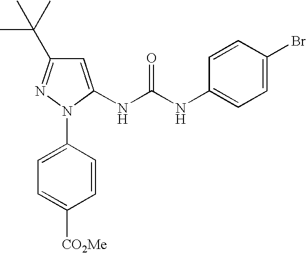

The title compound was synthesized in a manner analogous to Example 41 utilizing Example X (1.37 g) and p-bromophenylisocyanate (990 mg) to yield methyl 4-{3-tert-butyl-5-[3-(4-bromophenyl)ureido]-1H-pyrazol-1-yl}benzoate as white crystals (1.4 g, 59%). HPLC purity: 94%; mp: 270 272; ¹H NMR (DMSO-d₆): δ 9.2 (s, 1H), 8.6 (s, 1H), 8.1 (d, 2H), 7.7 (d, 2H), 7.4 (d, 4H), 6.4 (s, 1H), 3.9 (s, 3H), 1.3 (s, 9H).

### Example 60

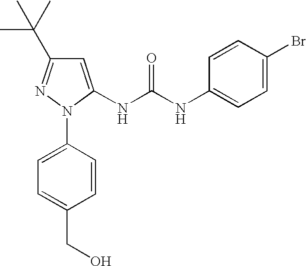

To a solution of Example 59 (700 mg) in 30 mL of toluene at −78° C., was added dropwise a solution of diisobutylaluminum hydride in toluene (1M in toluene, 7.5 mL) over 10 min. The reaction mixture was stirred for 30 min at −78° C., and then 30 min at 0° C. The reaction mixture was concentrated in vacuo to dryness and treated with H₂O. The solid was filtered and treated with acetonitrile. The solution was evaporated to dryness and the residue was dissolved in ethyl acetate, and precipitated by hexanes to afford yellow solid which was dried under vacuum to give 1-[3-tert-butyl-1-(4-hydroxymethyl)phenyl)-1H-pyrazol-5-yl]urea (400 mg, 61%). HPLC purity: 95%; ¹H NMR (DMSO-d₆): δ 9.2 (s, 1H), 8.4 (s, 1H), 7.5 (m, 8H), 6.4 (s, 1H), 5.3 (t, 1H), 4.6 (d, 2H), 1.3 (s, 9H). 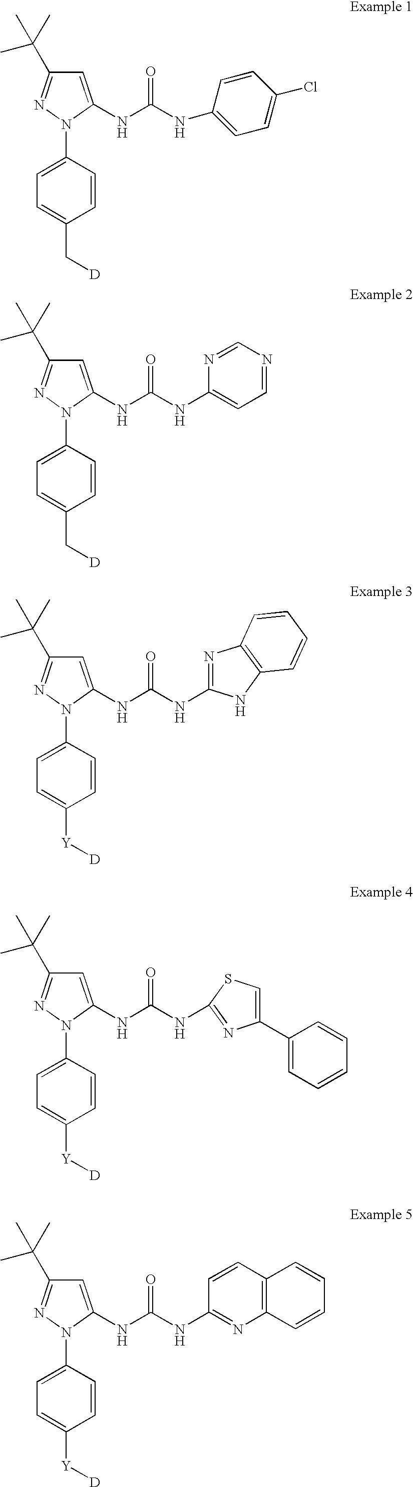 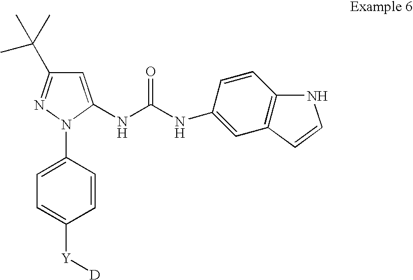
Wherein Y is O, S, NR6, —NR6SO2-, NR6CO—, alkylene, O—(CH2)n-, NR6-(CH2)n-, wherein one of the methylene units may be substituted with an oxo group, or Y is a direct bond; D is taken from the groups identified in Chart I: 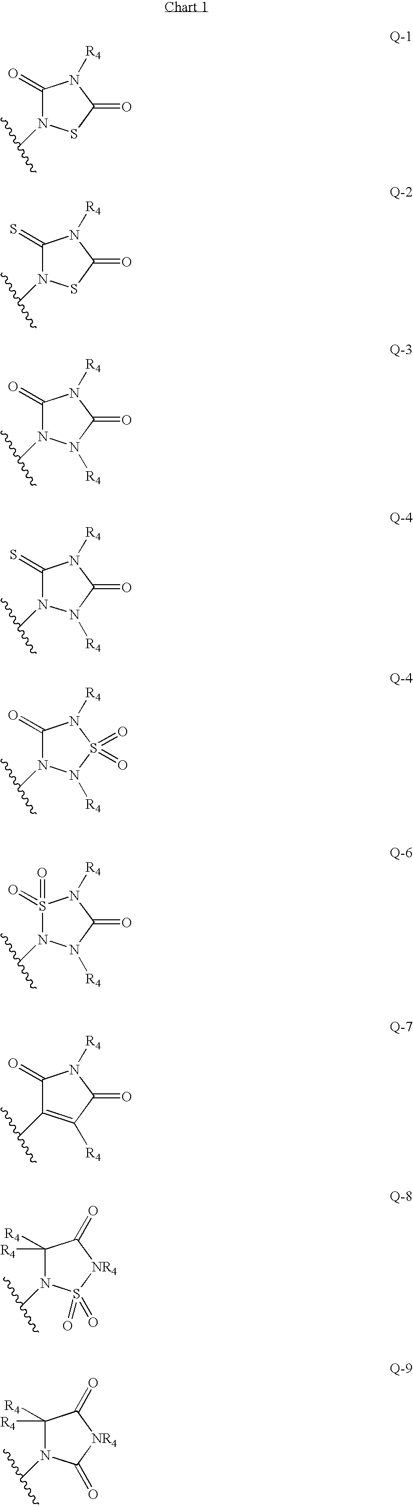 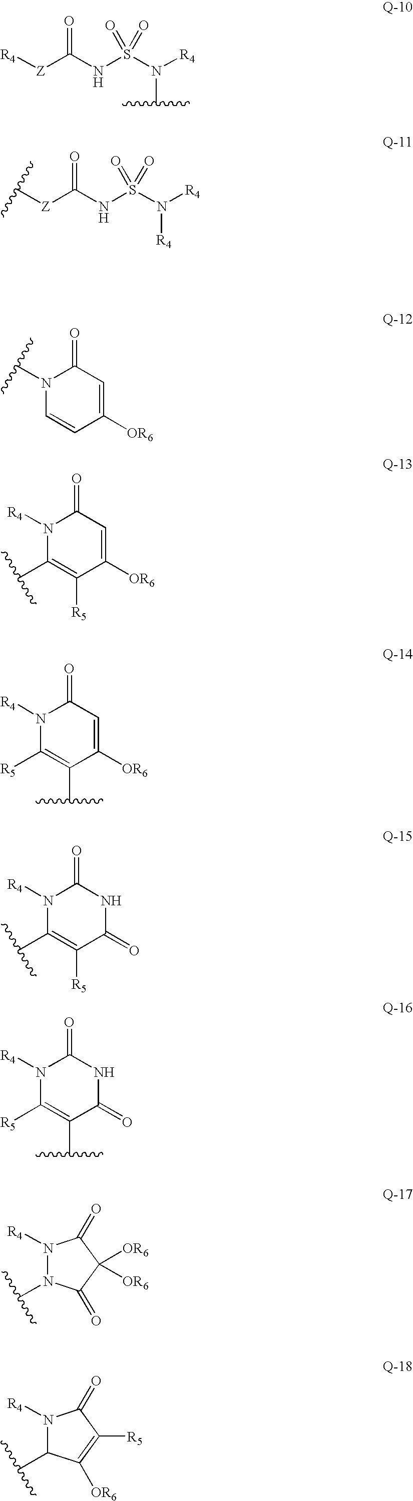 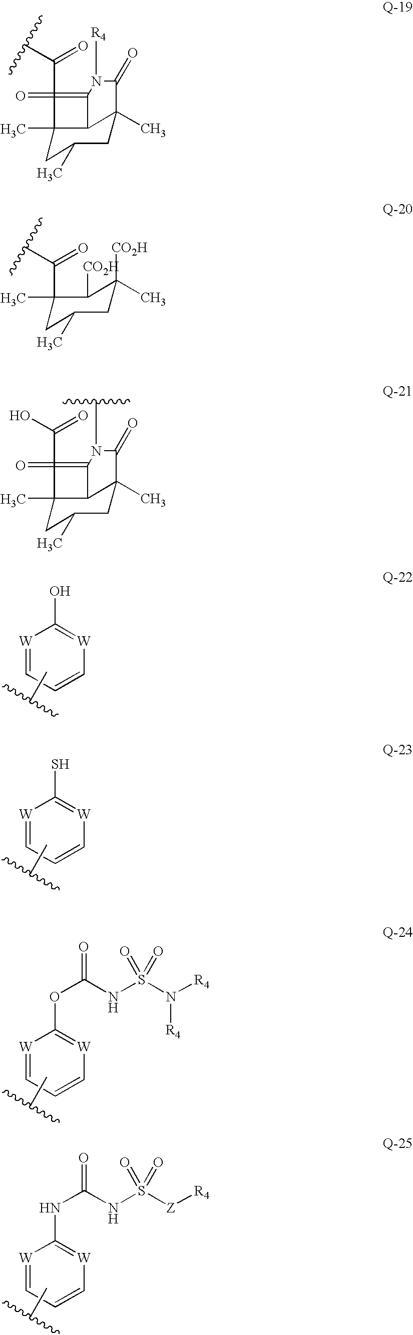 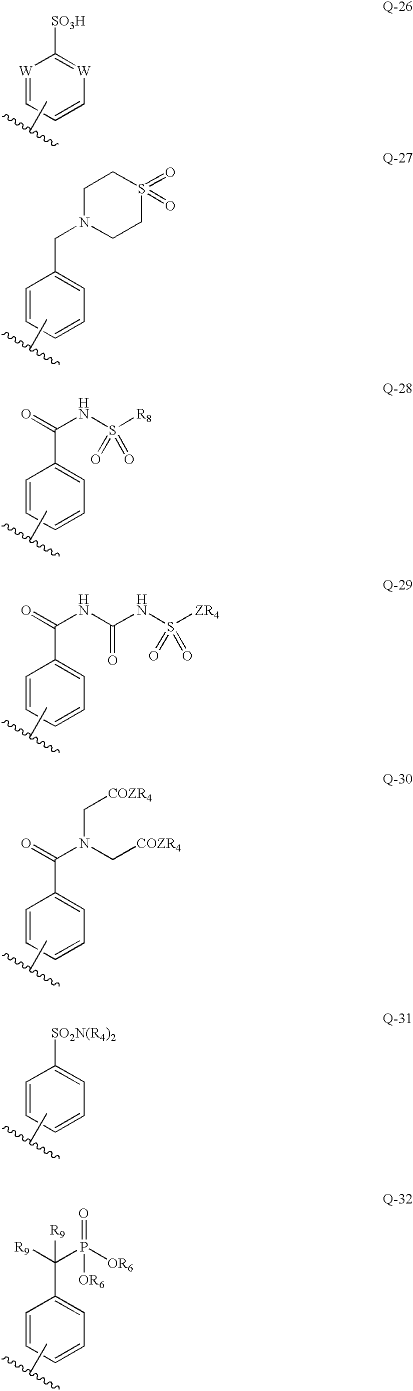 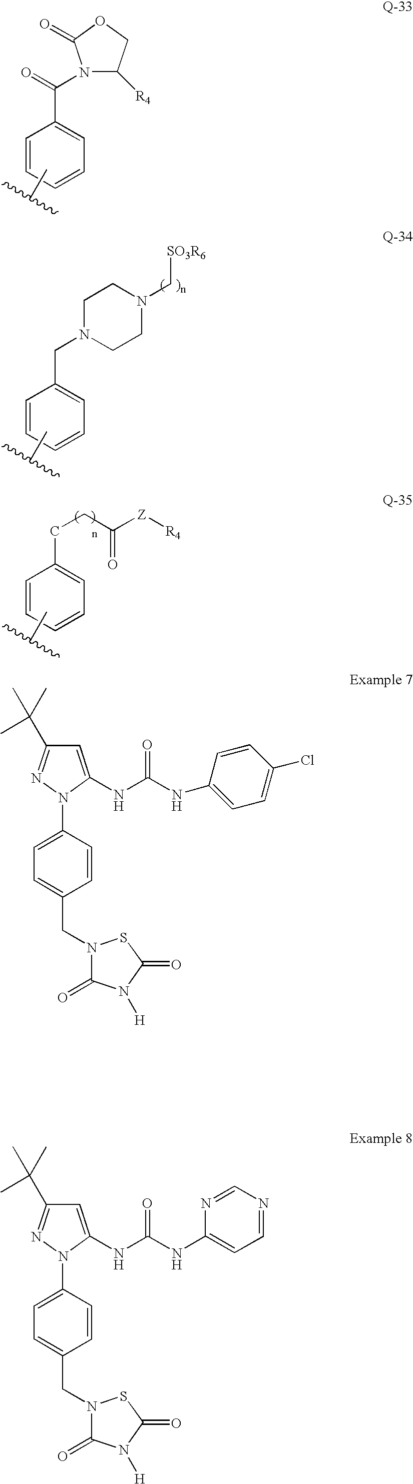 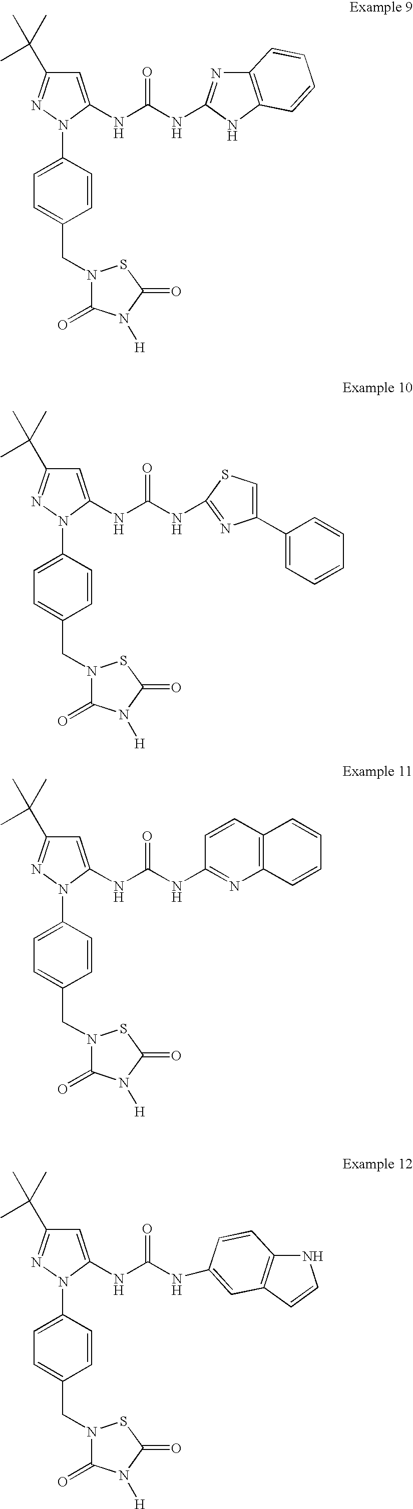 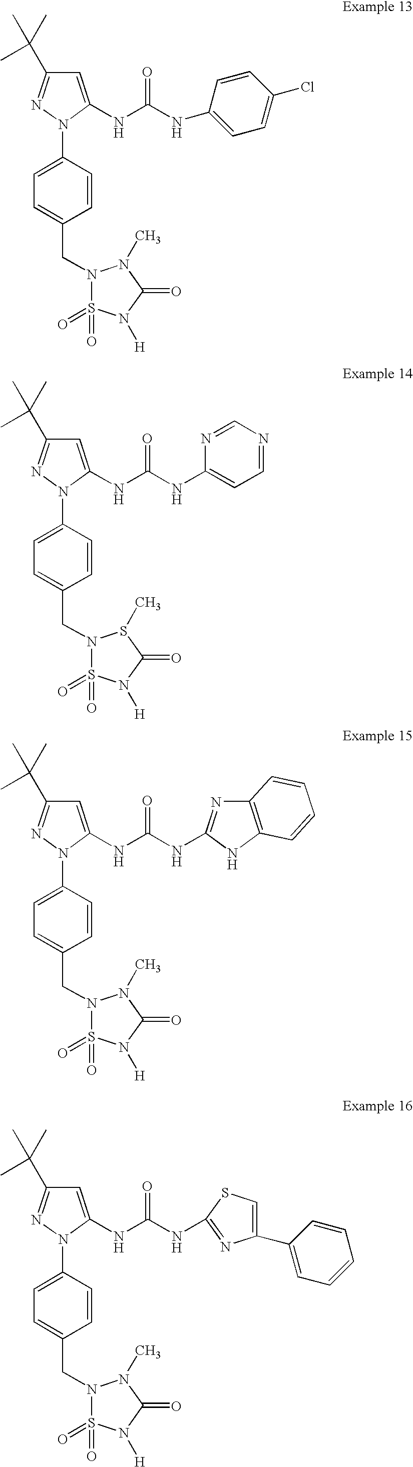 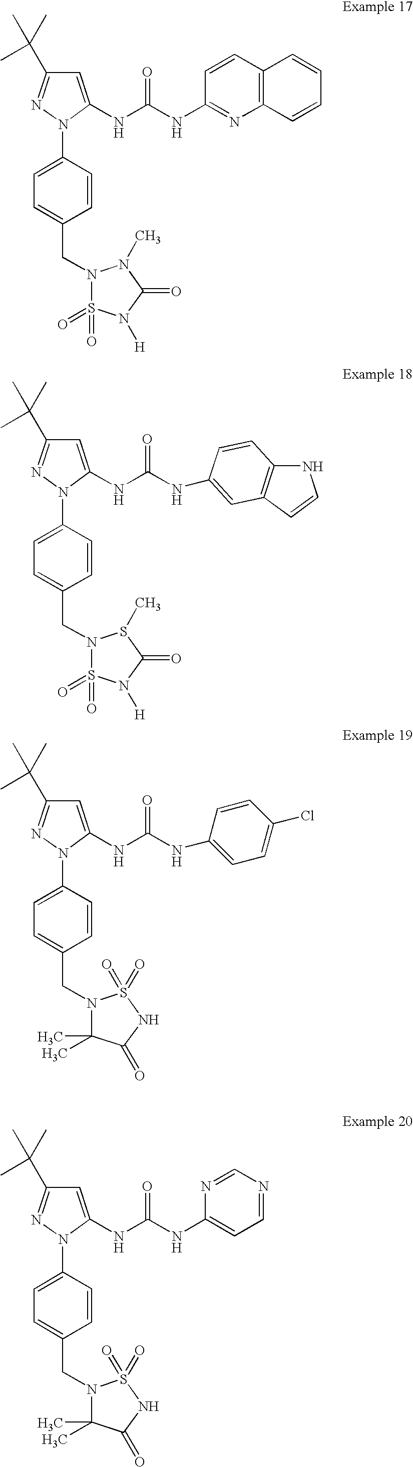 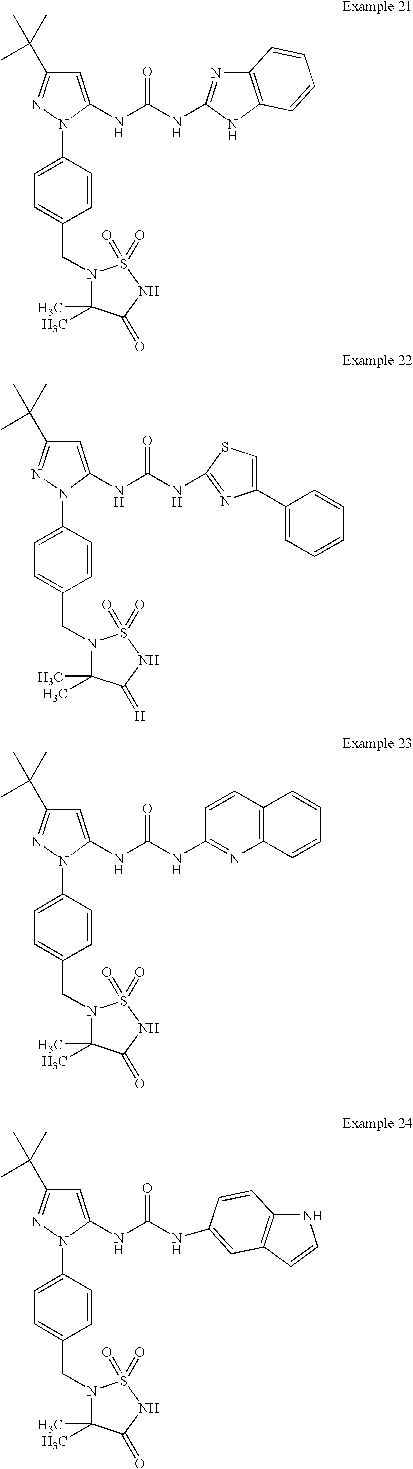 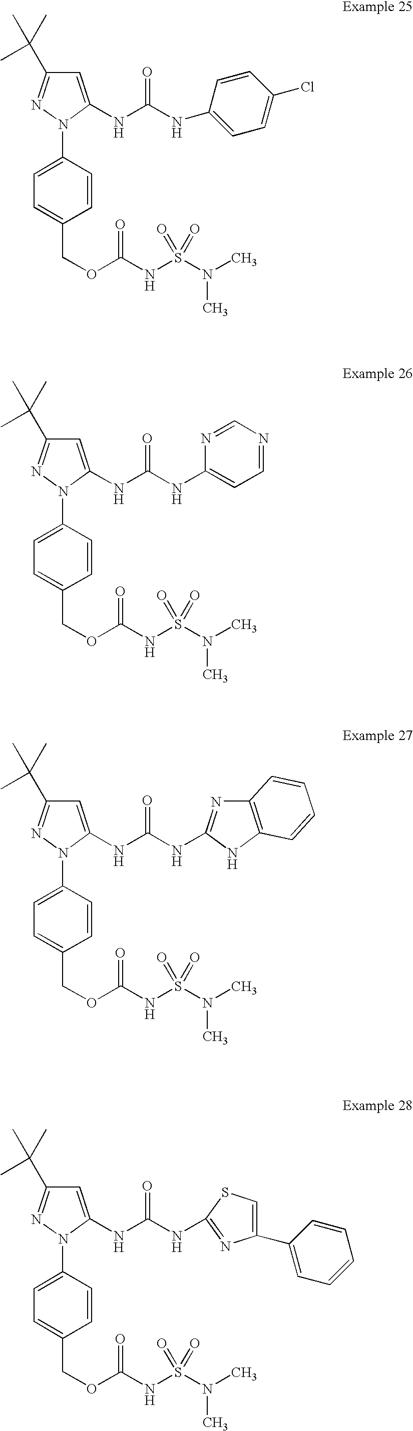 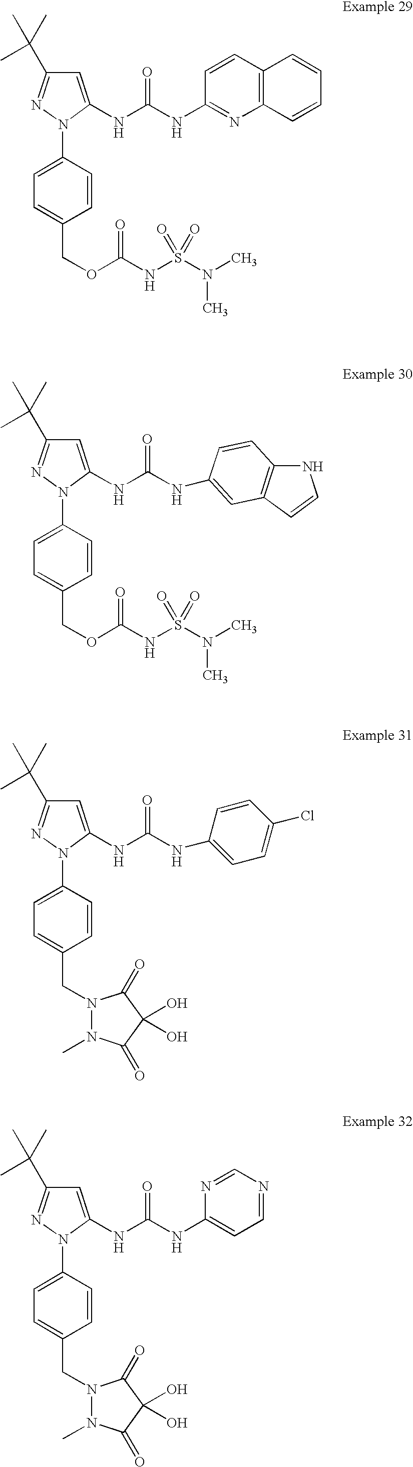 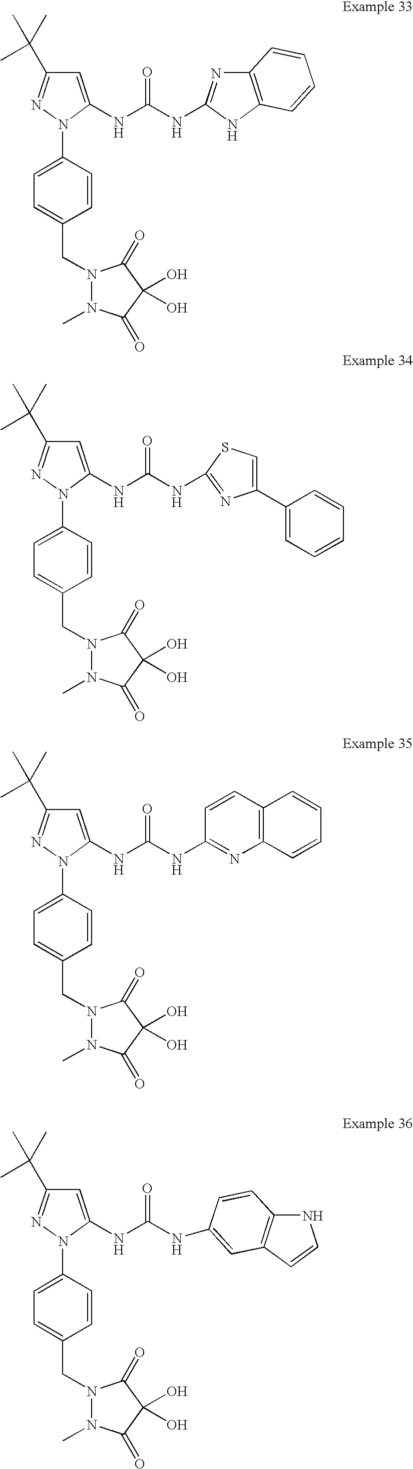 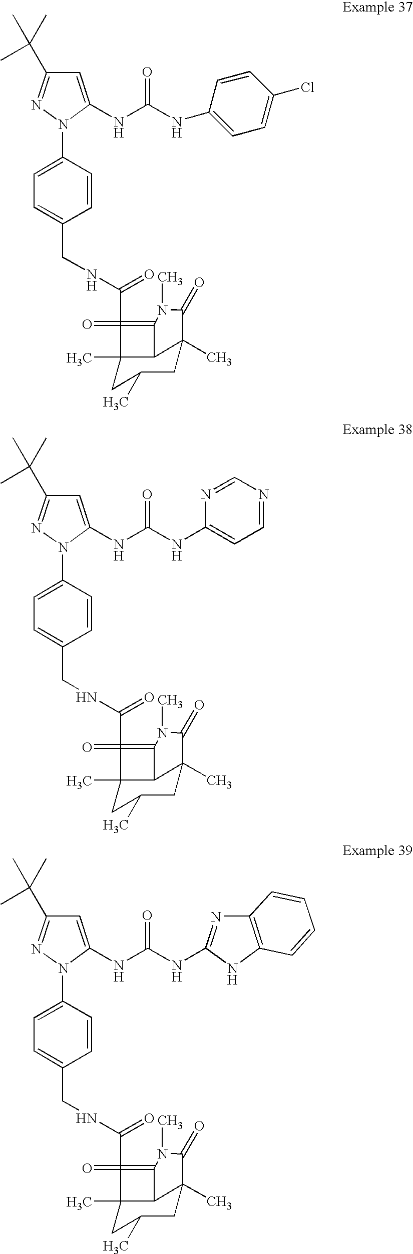 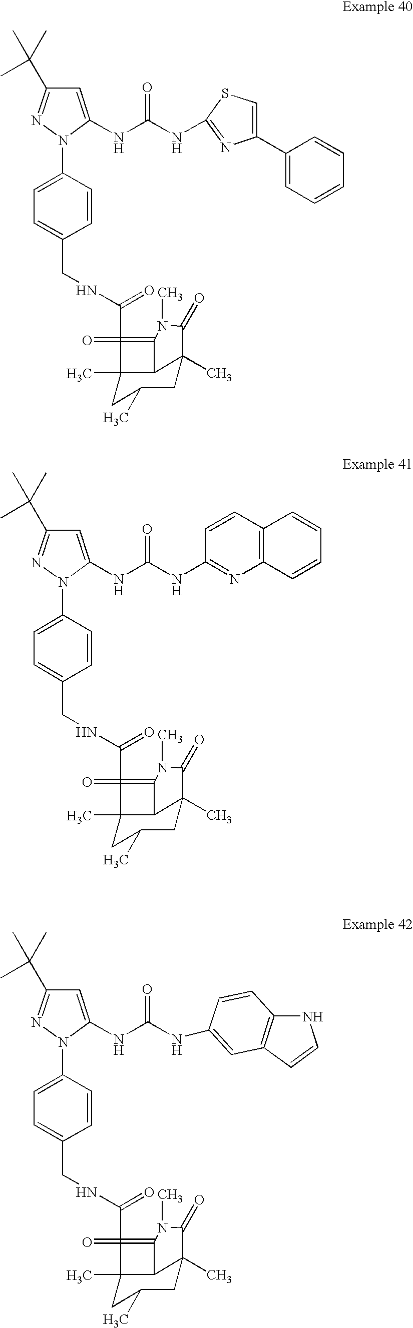 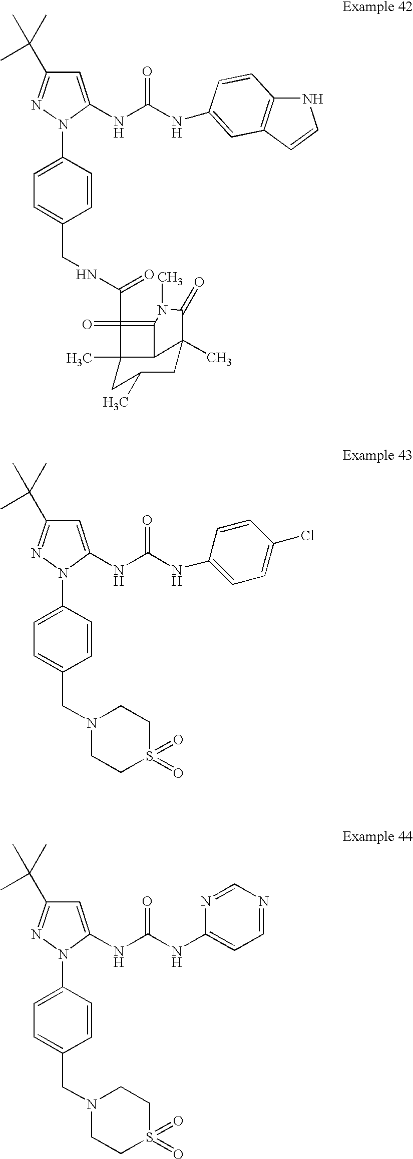 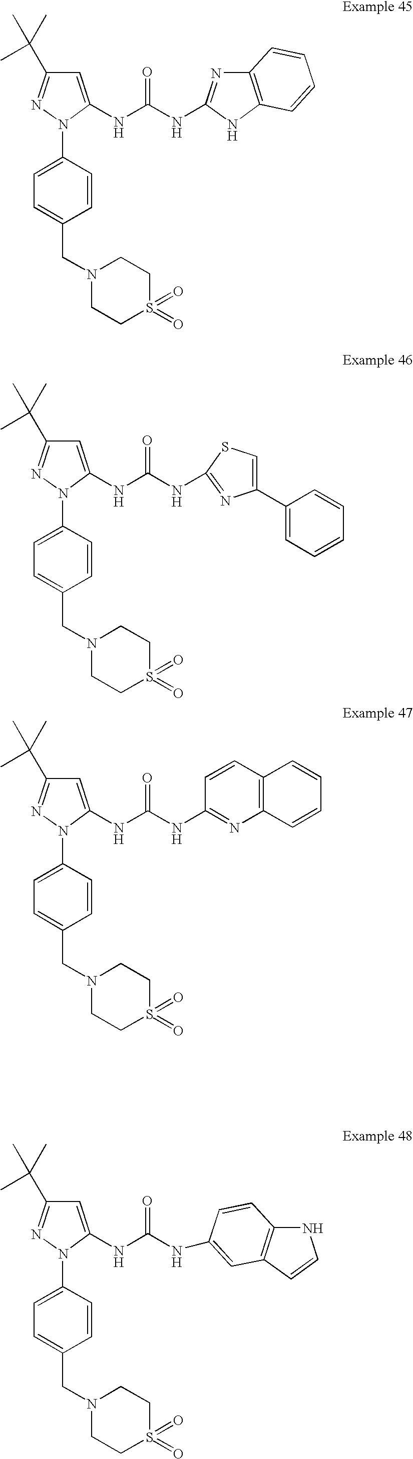 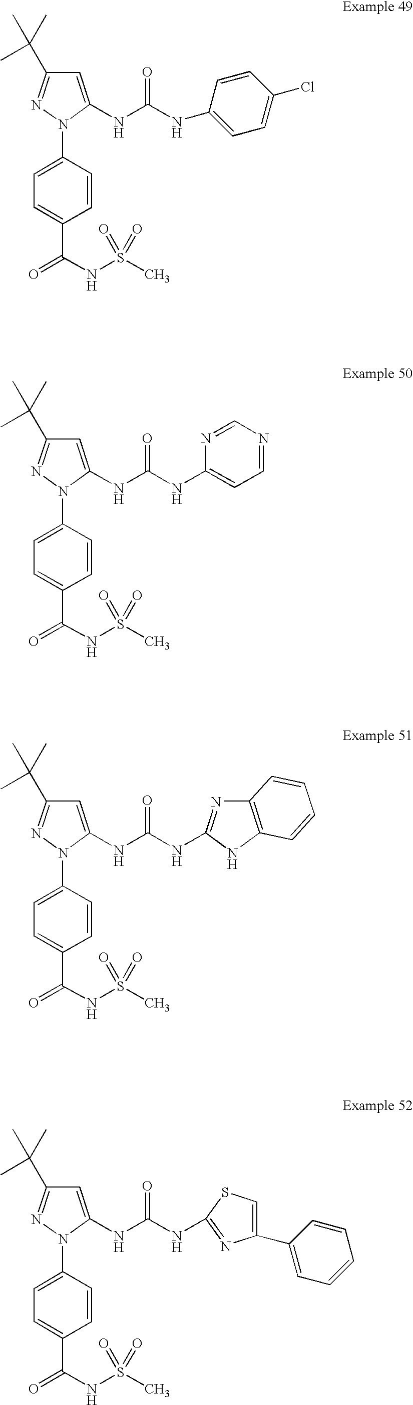 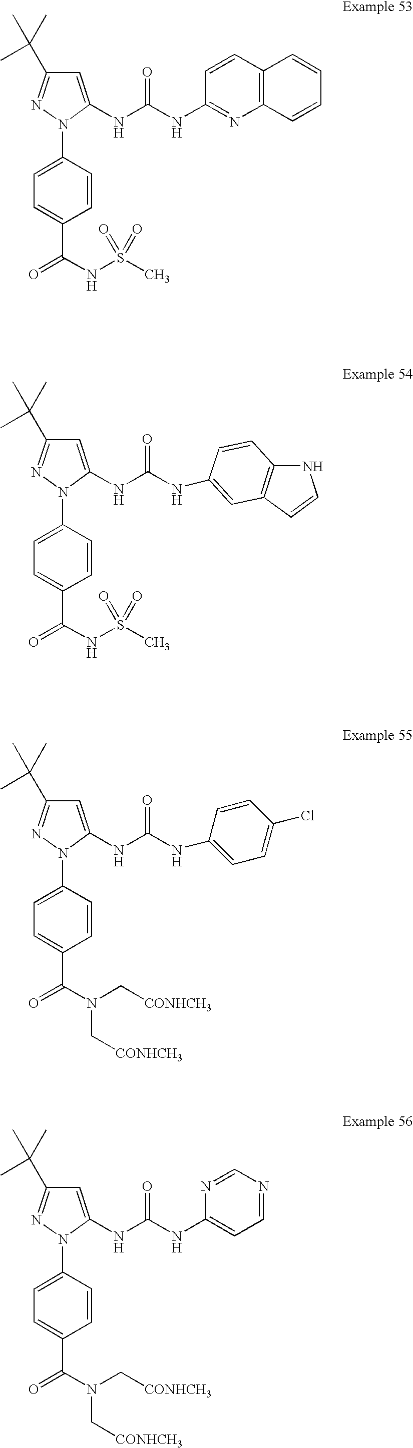 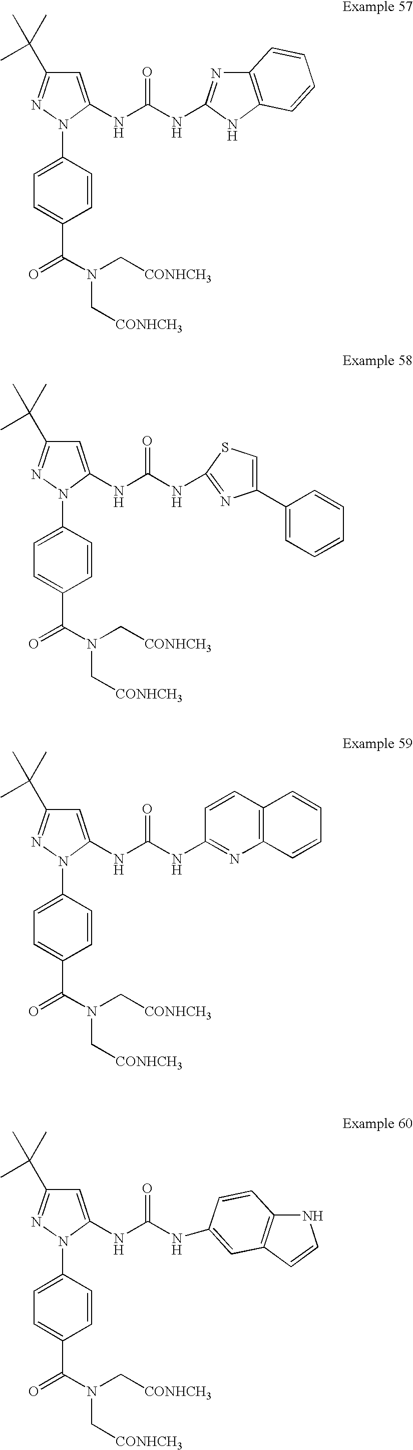 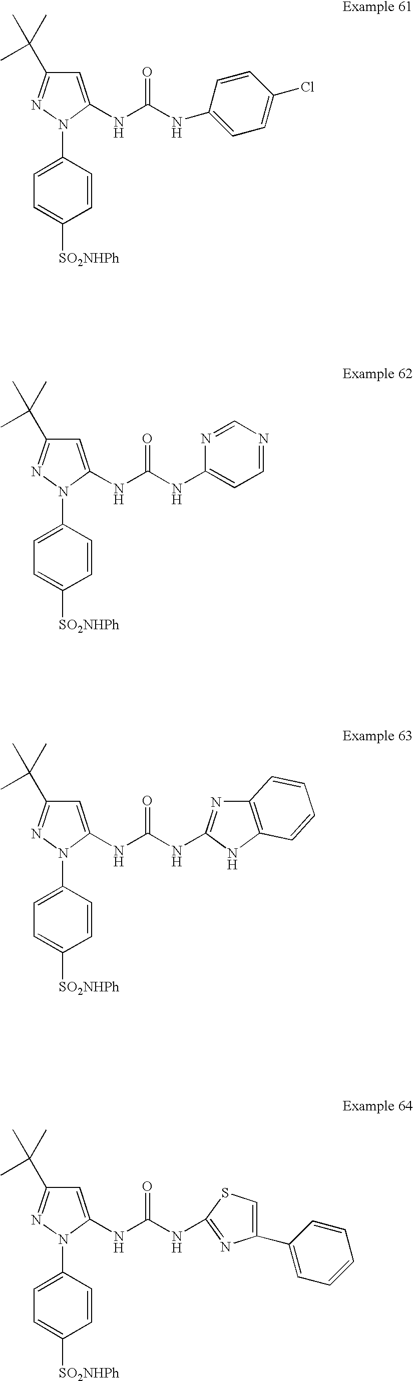 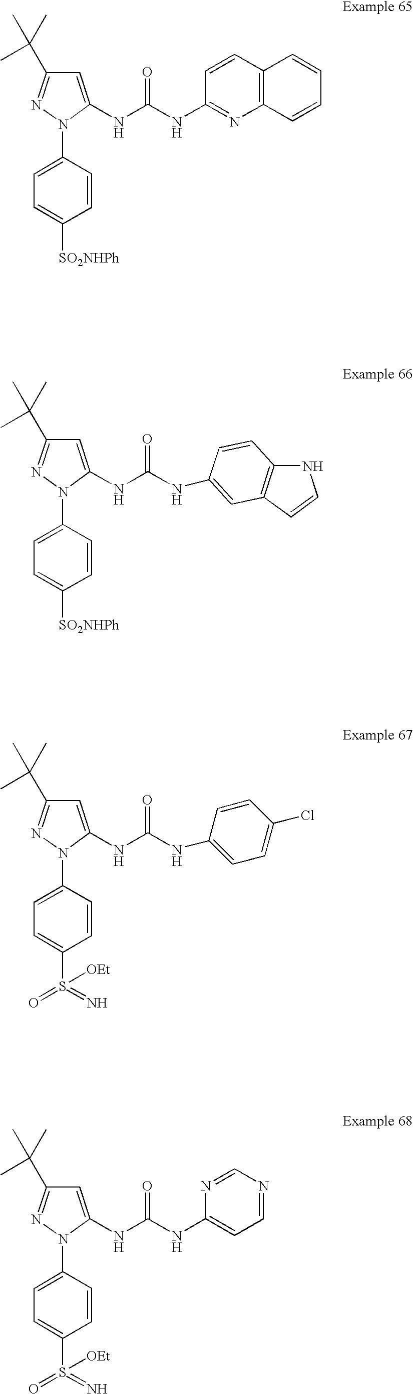 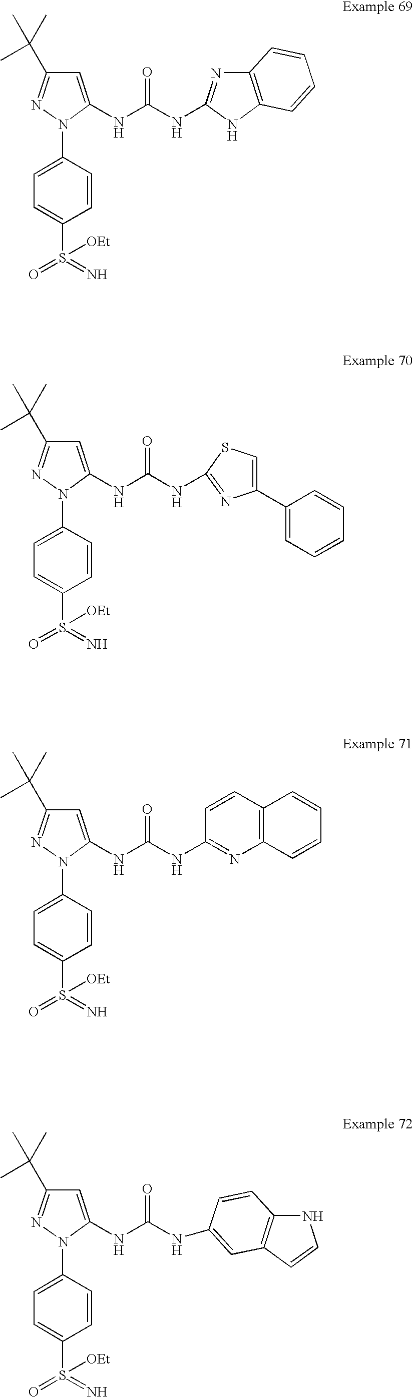 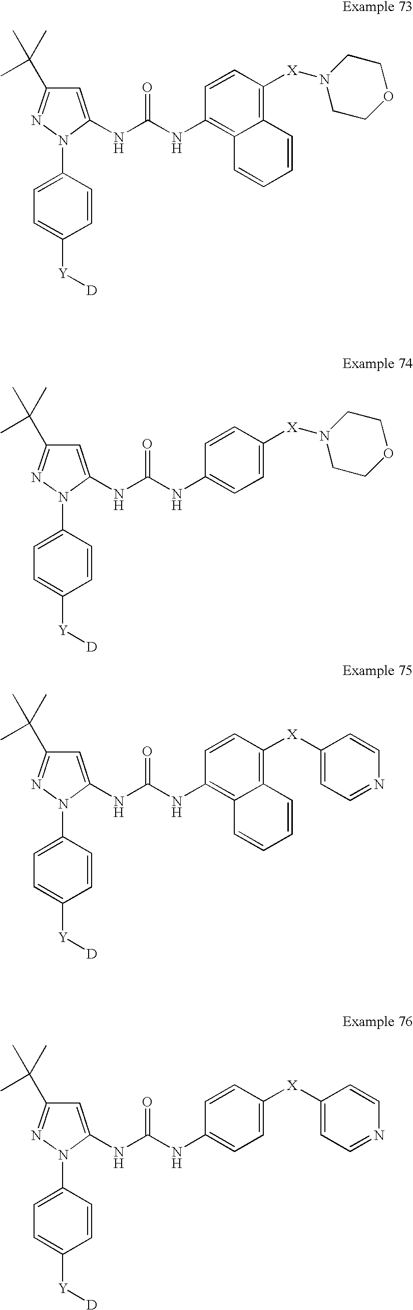 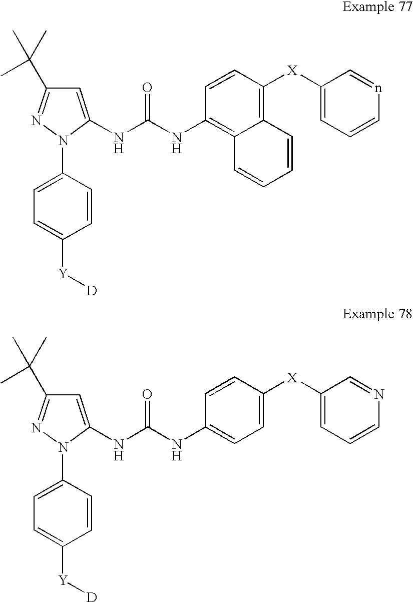
wherein X or Y is O, S, NR6, —NR6SO2-, NR6CO—, alkylene, O—(CH2)n-, NR6-(CH2)n-, wherein one of the methylene units may be substituted with an oxo group, or X or Y is a direct bond; D is taken from the groups identified in Chart I: 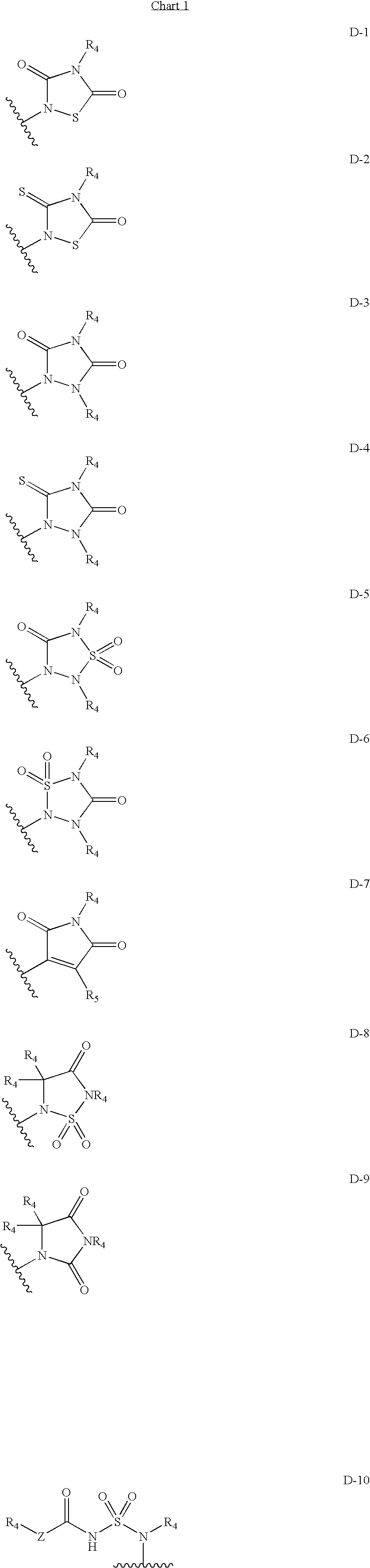 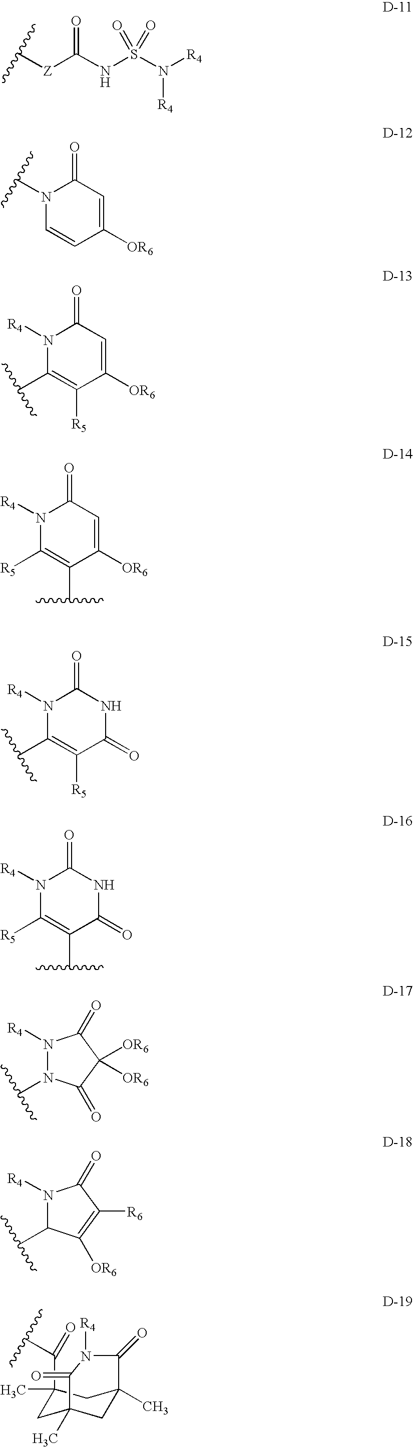 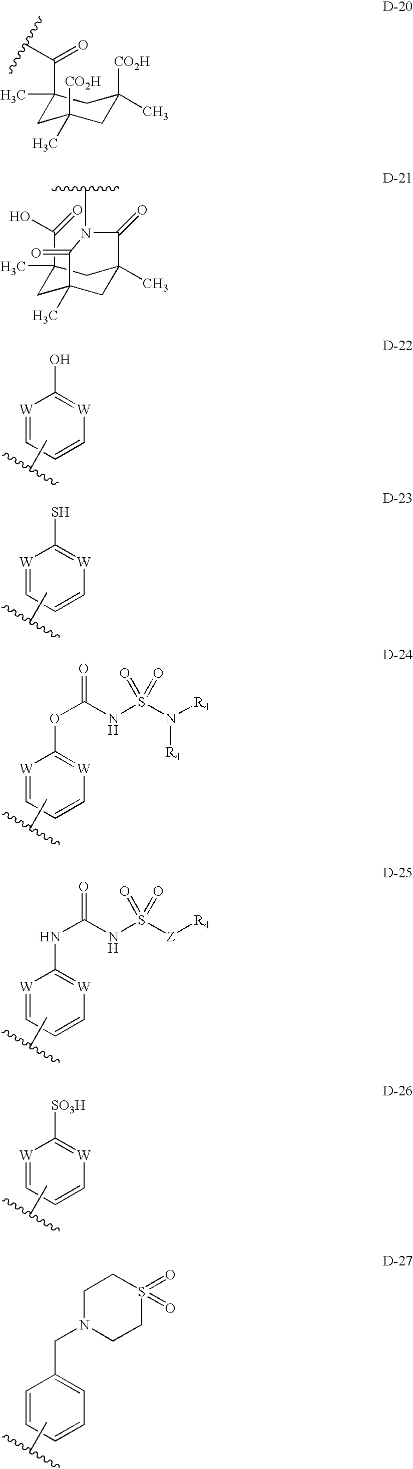 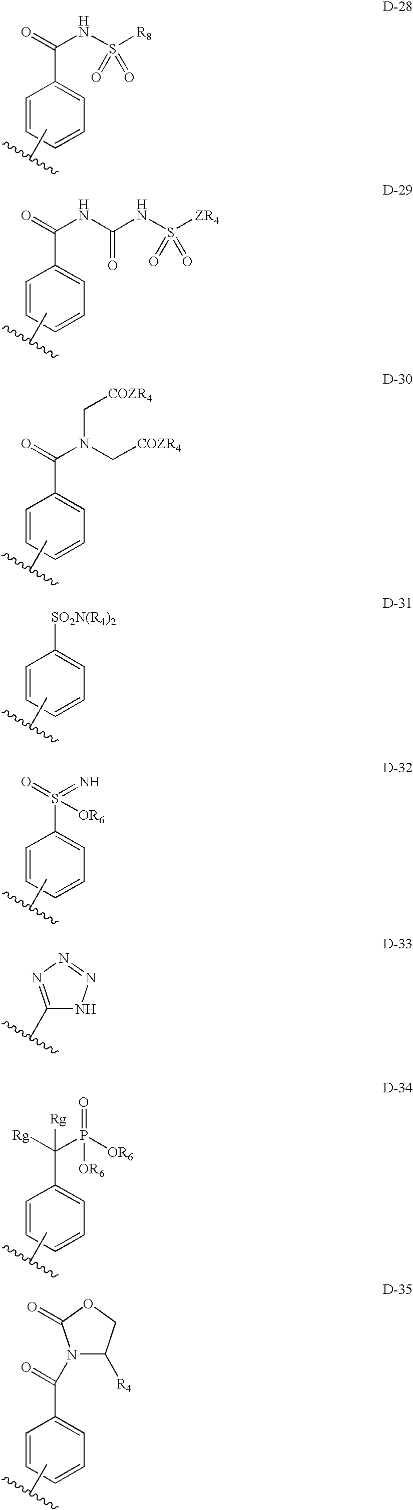 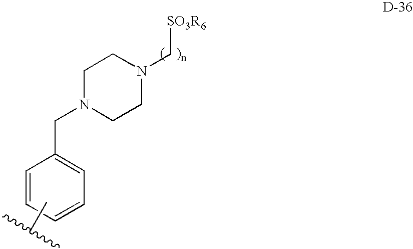
Specific examples of the present invention are illustrated by their structural formulae below:

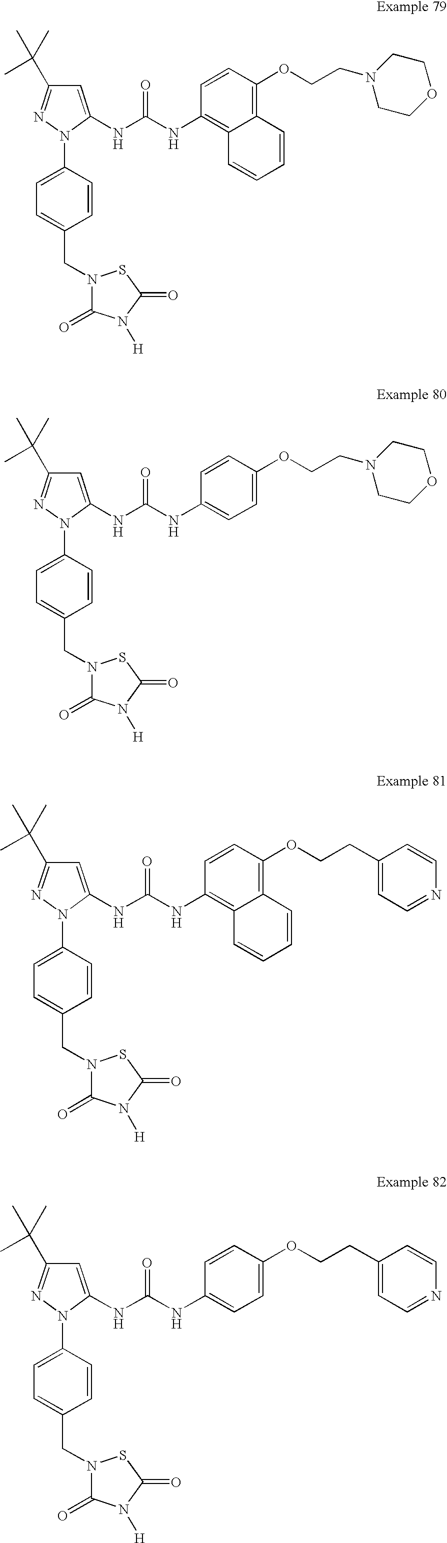

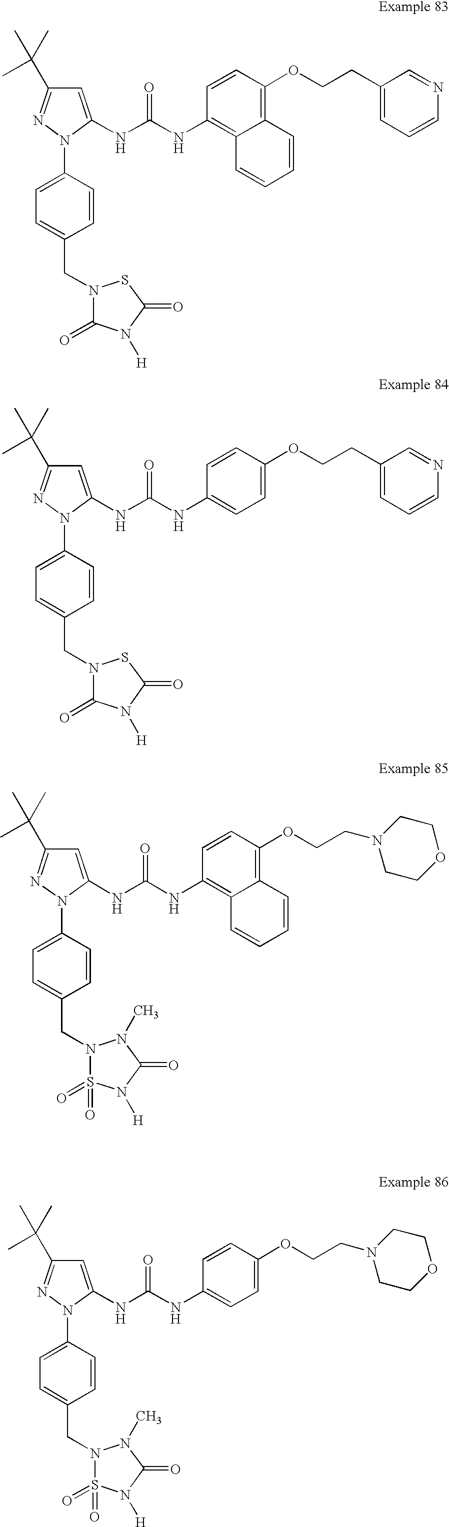

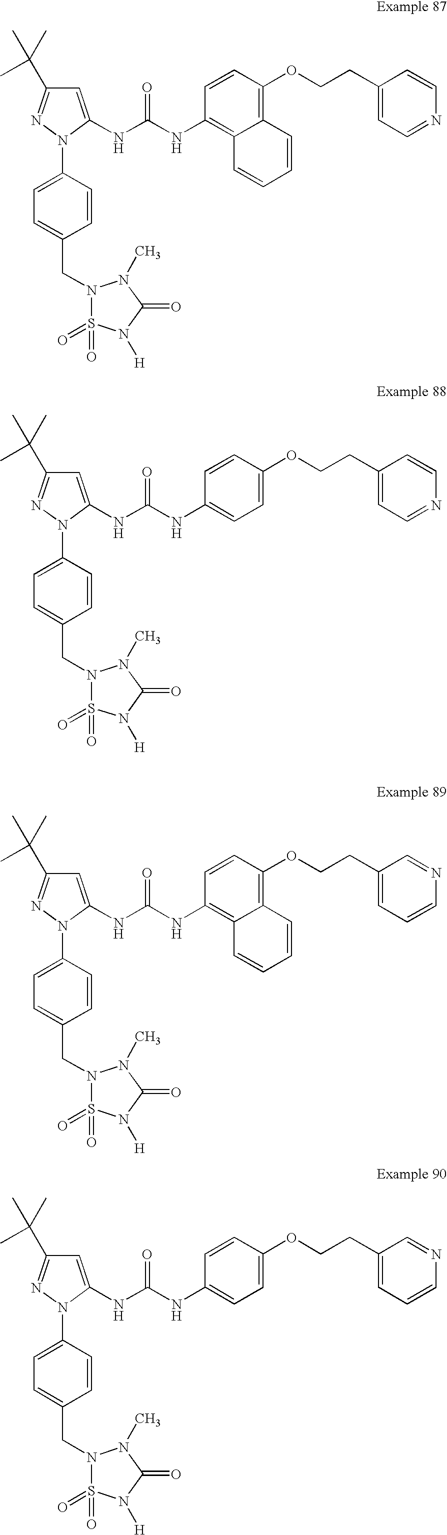

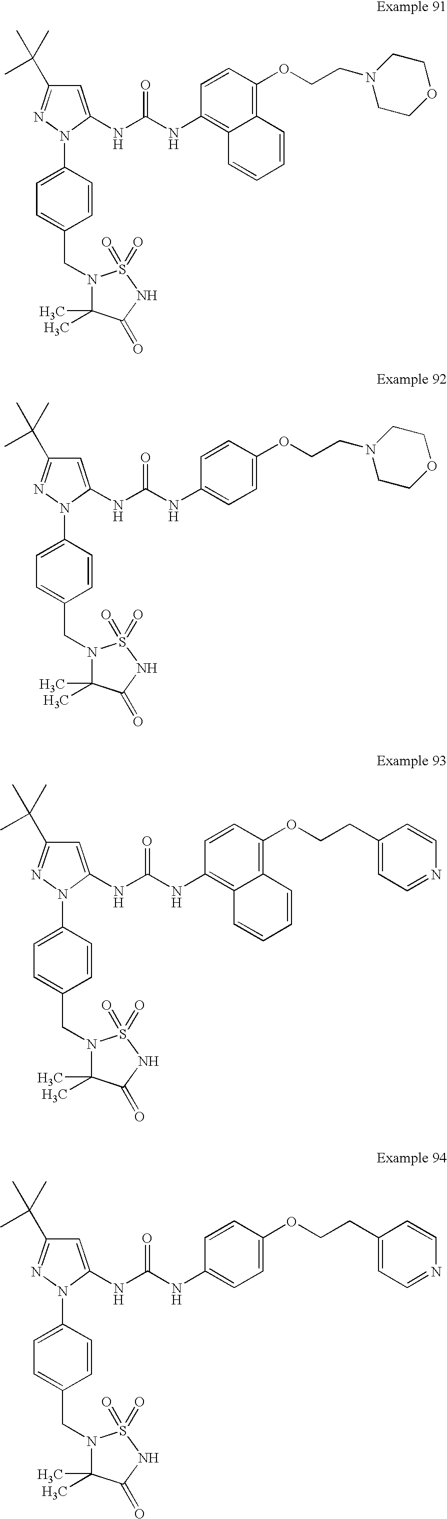

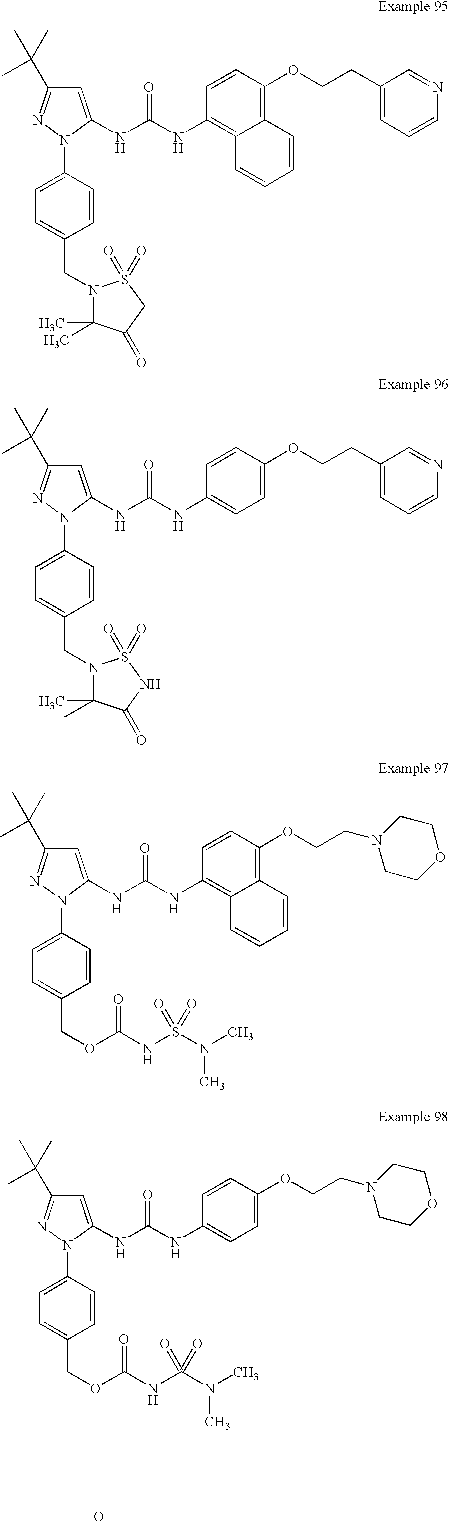

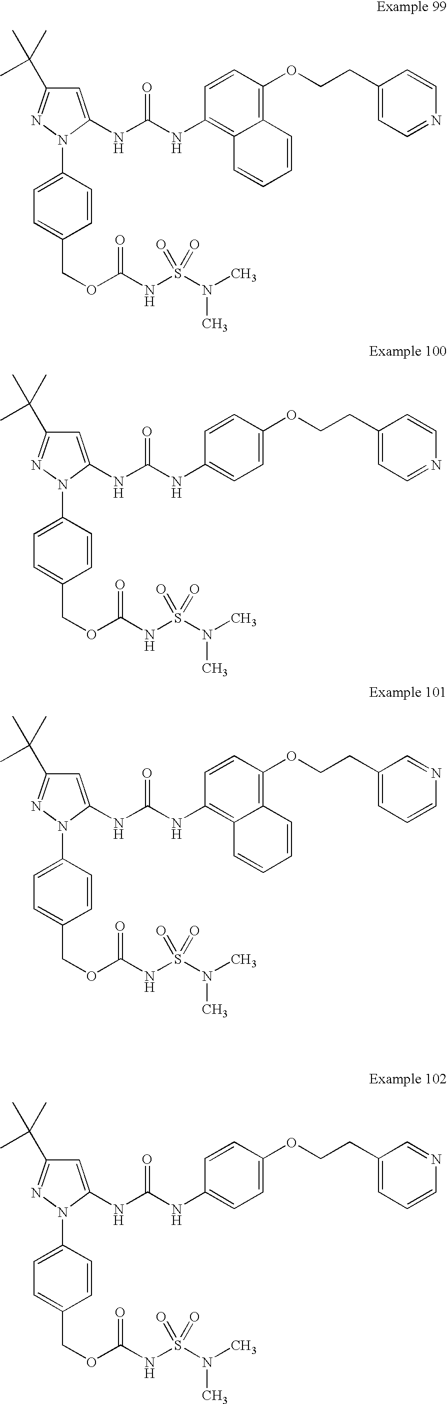

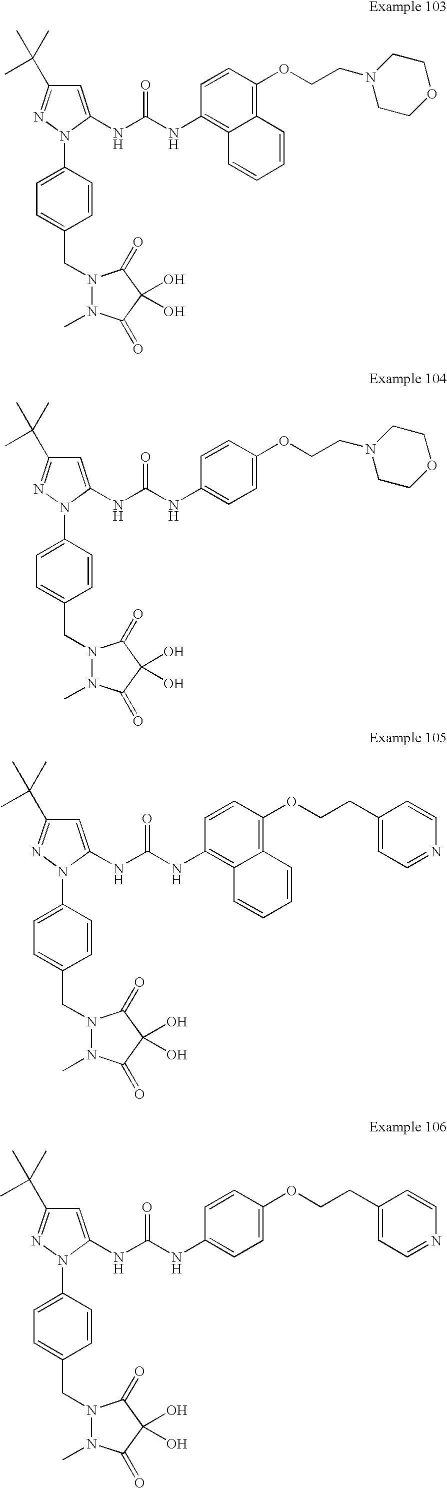

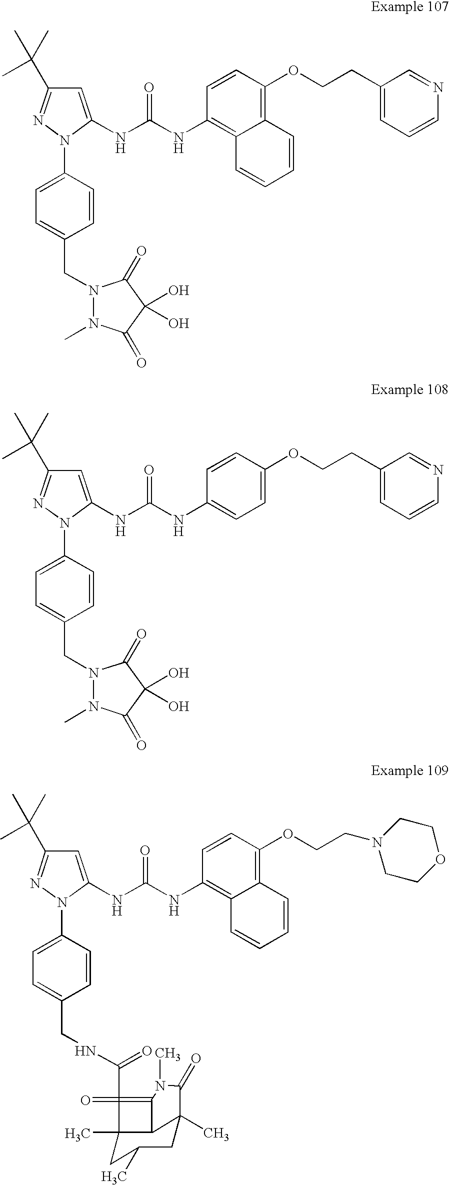

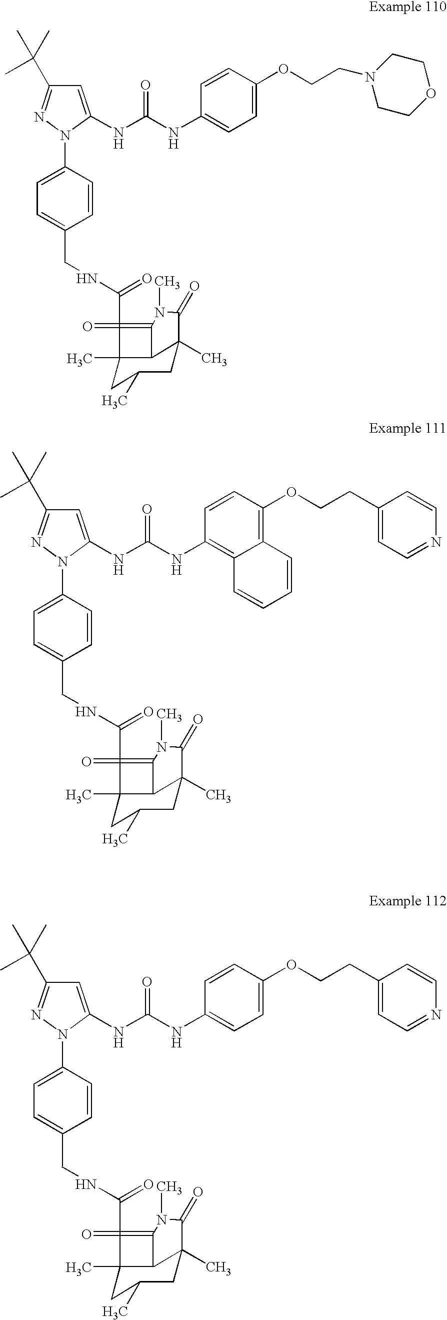

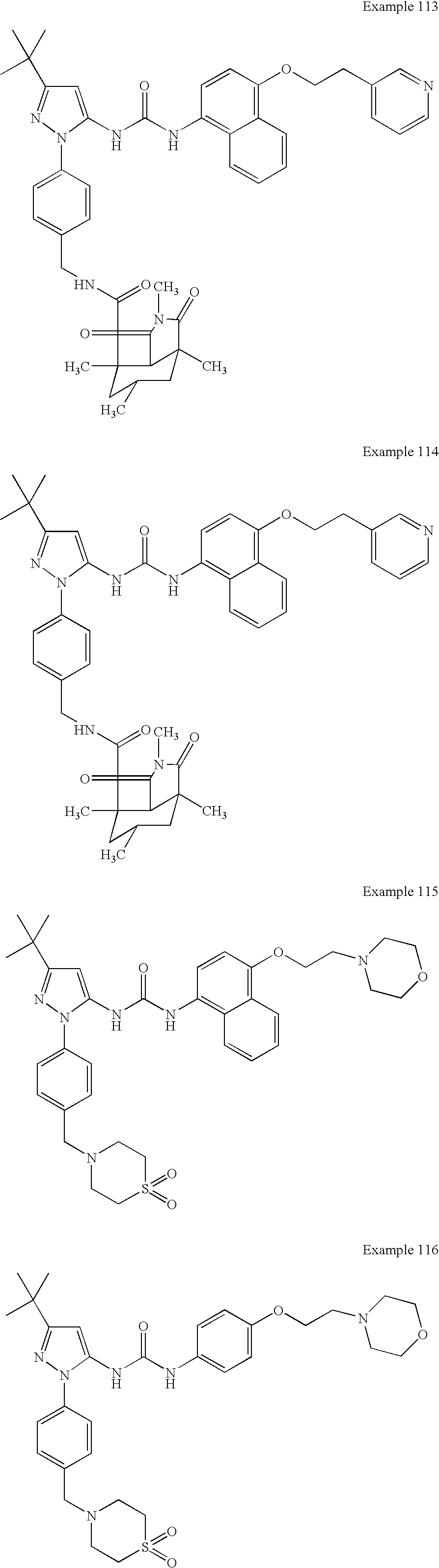

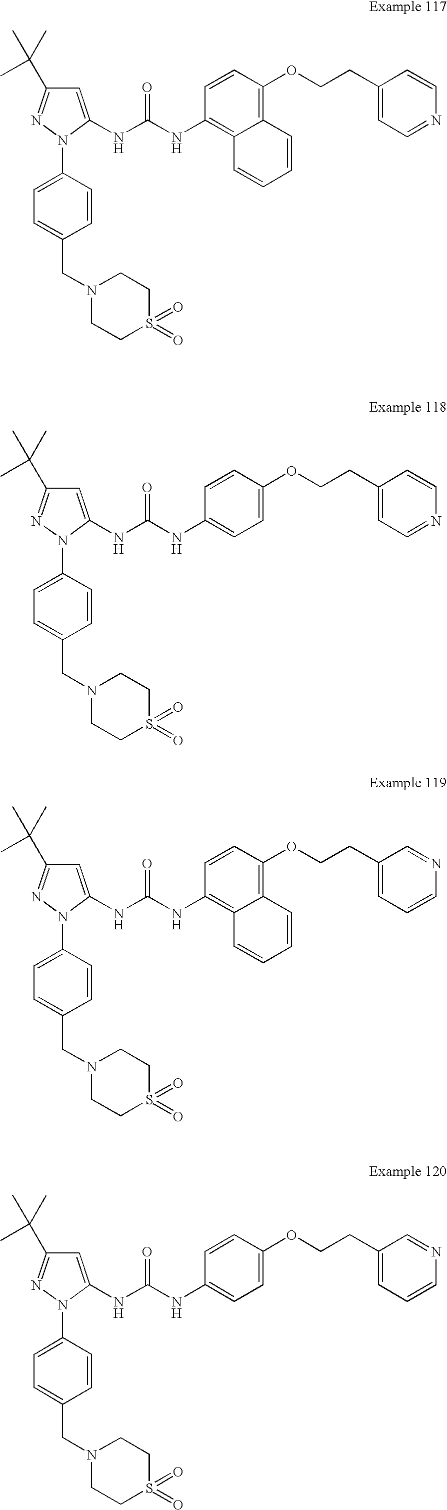

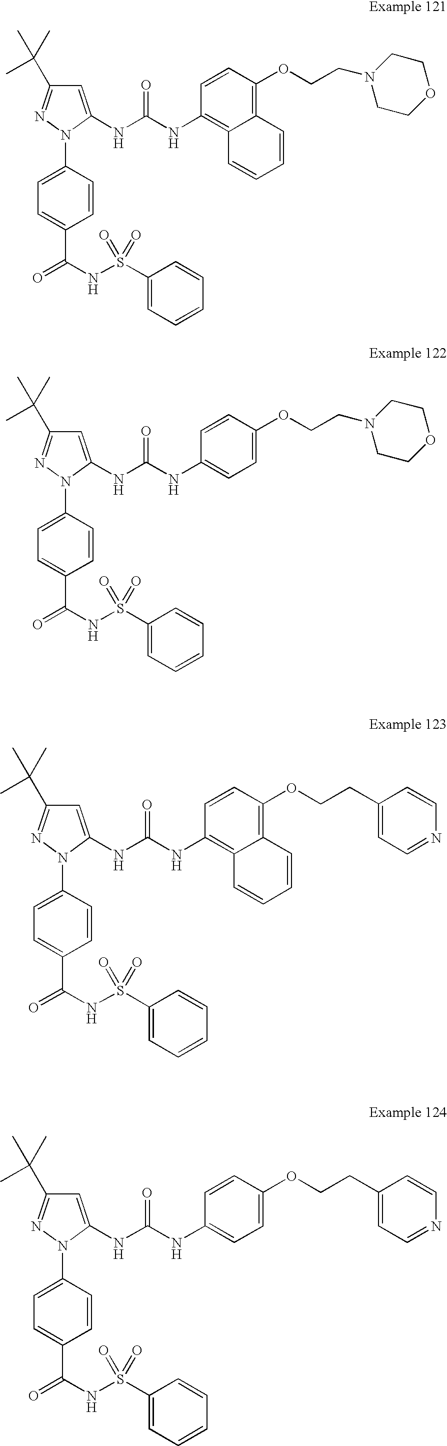

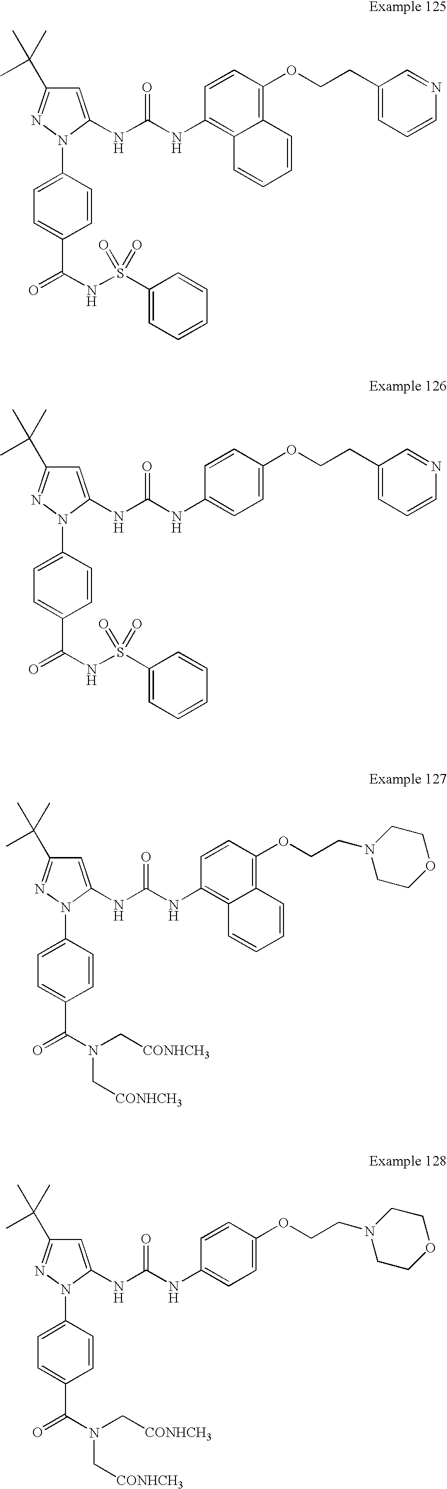

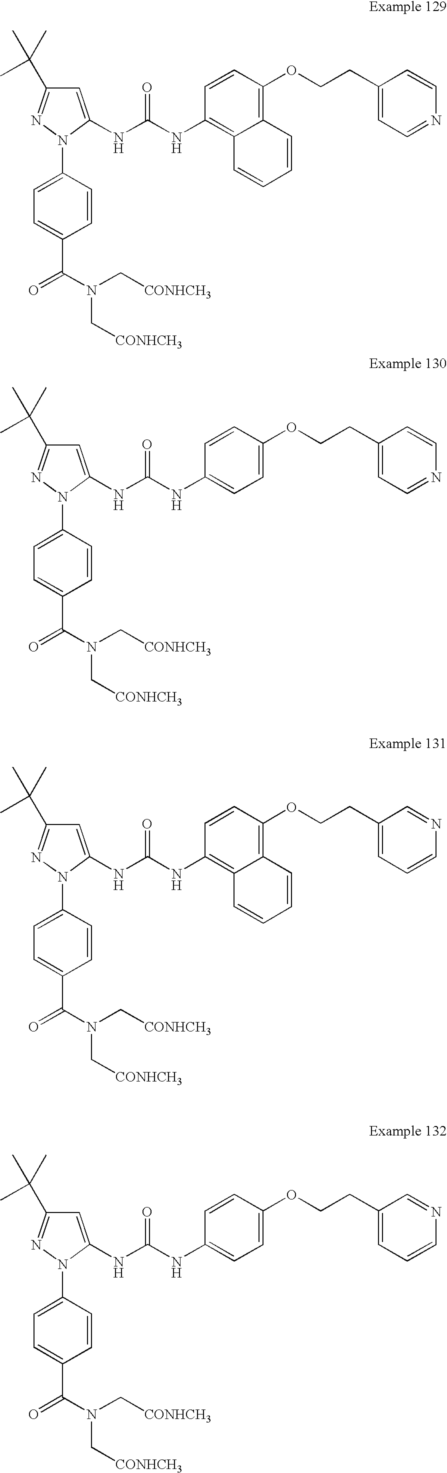

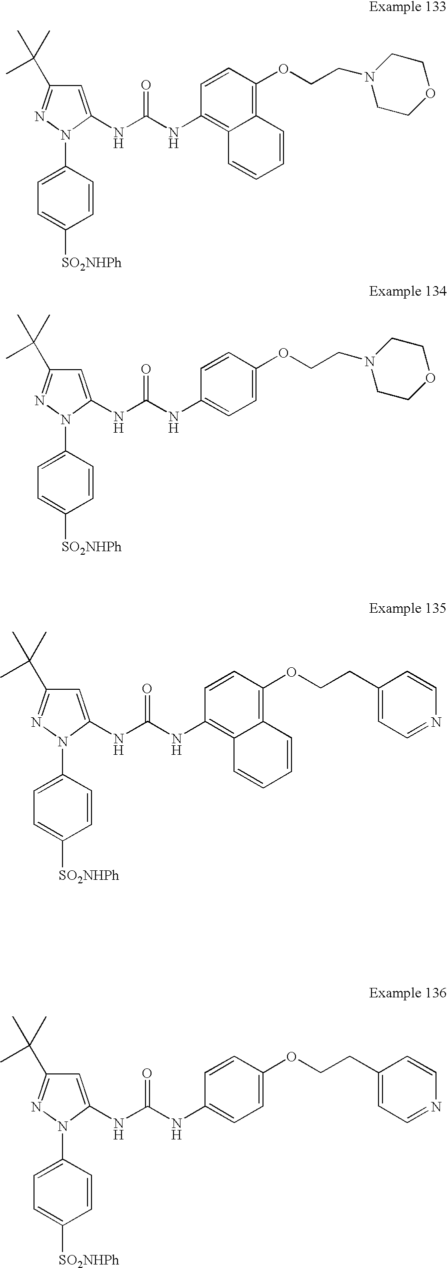

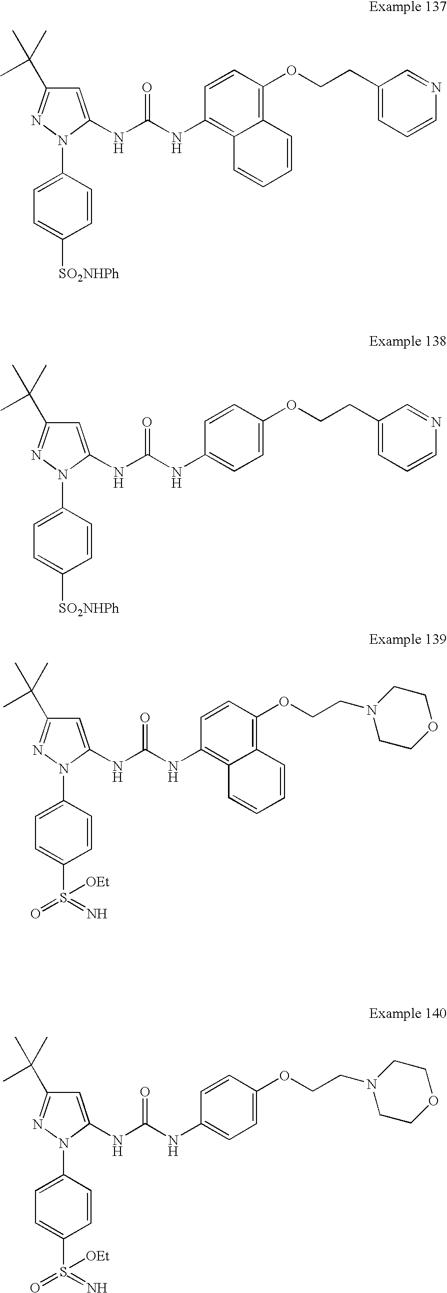

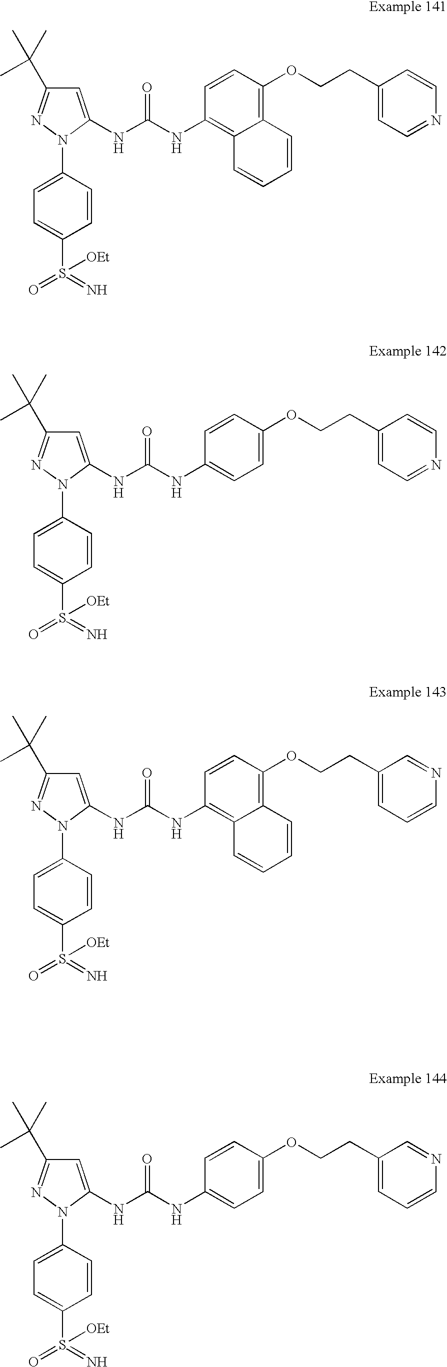

### Example Y

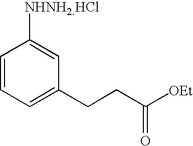

To a solution of 3-nitro-benzaldehyde (15.1 g, 0.1 mol) in CH₂Cl₂ (200 mL) was added (triphenyl-15-phosphanylidene)-acetic acid ethyl ester (34.8 g, 0.1 mol) in CH₂Cl₂ (100 mL) dropwise at 0° C., which was stirred for 2 h. After removal the solvent under reduced pressure, the residue was purified by column chromatography to afford 3-(3-nitro-phenyl)-acrylic acid ethyl ester (16.5 g, 74.6%) ¹H-NMR (400 M, CDCl₃): 8.42 (s, 1H), 8.23 (d, J=8.0 Hz, 1H), 7.82 (d, J=7.6 Hz, 1H), 7.72 (d, J=16.0 Hz, 1H), 7.58 (t, J=8.0 Hz, 1H), 6.56 (d, J=16.0 Hz, 1H), 4.29 (q, J=7.2 Hz, 2H), 1.36 (t, J=6.8 Hz, 3H).

A mixture of 3-(3-nitro-phenyl)-acrylic acid ethyl ester (16.5 g, 74.6 mmol) and Pd/C (1.65 g) in methanol (200 mL) was stirred under 40 psi of H₂ at RT for 2 h then filtered over celite. After removal the solvent, 14 g of 3-(3-amino-phenyl)-propionic acid ethyl ester was obtained and used directly without further purification. ¹H-NMR (400 MHz, CDCl₃): 7.11 (t, J=5.6 Hz, 1H), 6.67 (d, J=7.2 Hz, 1H), 6.63-6.61 (m, 2H), 4.13 (q, J=7.2 Hz, 2H), 2.87 (t, J=8.0 Hz, 2H), 2.59 (t, J=7.6 Hz, 2H), 1.34 (t, J=6.8 Hz, 3H).

To a solution of 3-(3-amino-phenyl)-propionic acid ethyl ester (14 g, 72.5 mmol) in concentrated HCl (200 mL) was added an aqueous solution (10 mL) of NaNO₂ (5 g, 72.5 mmol) at 0° C. and the resulting mixture was stirred for 1 h. A solution of SnCl₂.2H₂O (33 g, 145 mmol) in concentrated HCl (150 mL) was then added at 0° C. The reaction solution was stirred for an additional 2 h at RT. The precipitate was filtered and washed with ethanol and ether to give 3-(3-hydrazino-phenyl)-propionic acid ethyl ester as a white solid, which was used without further purification.

### Example Z

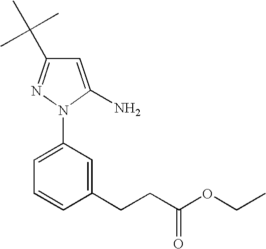

A mixture of Example Y (13 g, 53.3 mmol) and 4,4-dimethyl-3-oxo-pentanenitrile (6.9 g, 55 mol) in ethanol (150 mL) was heated to reflux overnight. The reaction solution was evaporated under reduced pressure. The residue was purified by column chromatography to give 3-[3-(5-amino-3-t-butyl-pyrazol-1-yl)-phenyl]-propionic acid ethyl ester (14.3 g, 45.4 mmol) as a white solid. ¹H NMR (DMSO-d₆): 7.39-7.32 (m, 3H), 7.11 (d, J=6.8 Hz, 1H), 5.34 (s, 1H), 5.16 (s, 2H), 4.03 (q, J=7.2 Hz, 2H), 2.88 (t, J=7.6 Hz, 2H), 2.63 (t, J=7.6 Hz, 2H), 1.19 (s, 9H), 1.15 (t, J=7.2 Hz, 3H).

### Example 145

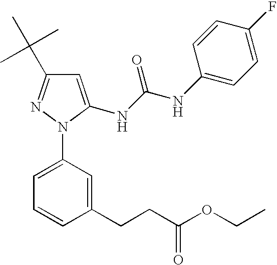

A solution of 4-fluoro-phenylamine (111 mg, 1.0 mmol) and CDI (165 mg, 1.0 mmol) in DMF (2 mL) was stirred at RT for 30 min, and was then added to a solution of Example Z (315 mg, 1.0 mmol) in DMF (2 mL). The resulting mixture was stirred at RT overnight then added to water (50 mL). The reaction mixture was extracted with ethyl acetate (3×50 mL) and the combined organic extracts were washed with brine, dried (NaSO₄) and filtered. After concentrated under reduced pressure, the residue was purified by flash chromatography to afford 3-(3-{3-t-butyl-5-[3-(4-fluoro-phenyl)-ureido]-pyrazol-1-yl}-phenyl)-propionic acid ethyl ester (150 mg, 33%). ¹H-NMR (CDCl₃): 7.91 (s, 1H), 7.42 (d, J=4.8 Hz, 1H), 7.37-7.34 (m, 2H), 7.28 (s, 1H), 7.17-7.16 (m, 2H), 6.98 (t, J=8.8 Hz, 2H), 6.59 (s, 1H), 4.04 (q, J=7.2 Hz, 2H), 3.03 (t, J=7.2 Hz, 2H), 2.77 (t, J=7.2 Hz, 2H), 1.36 (s, 9H), 1.17 (t, J=7.2 Hz, 3H); MS (ESI) m/z: 453 (M+H⁺).

### Example 146

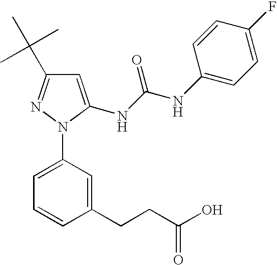

A solution of Example 145 (45 mg, 0.1 mmol) and 2N LiOH (3 mL) in MeOH (3 mL) was stirred at RT overnight. The reaction mixture was neutralized to pH=4, extracted with ethyl acetate (3×20 mL), the combined organic extracts were washed with brine, dried (NaSO₄) and filtered. The filtrate was concentrated to afford 3-(3-{3-t-butyl-5-[3-(4-fluoro-phenyl)-ureido]-pyrazol-1-yl}-phenyl)-propionic acid, (37 mg, 90%). ¹H NMR (CD₃OD): 7.63-7.62 (m, 2H), 7.56 (s, 1H), 7.53-7.48 (m, 1H), 7.41-7.38 (m, 2H), 7.04 (t, J=8.8 Hz, 2H), 5.49 (s, 1H), 3.07 (t, J=7.6 Hz, 2H), 2.72 (t, J=7.6 Hz, 2H), 1.42 (s, 9H); MS (ESI) m/z: 415 (M+H⁺).

### Example 147

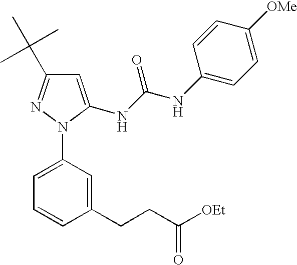

A mixture of 4-methoxy-phenylamine (123 mg, 1.0 mmol) and CDI (165 mg, 1.0 mmol) in DMF (2 mL) was stirred at RT for 30 min, and was then added a solution of Example Z (315 mg, 1.0 mmol) in DMF (2 mL). The resulting mixture was stirred at RT overnight then quenched with of water (50 mL). The reaction mixture was extracted with ethyl acetate (3×50 mL) and the combined organic extracts were washed with brine, dried (NaSO₄), filtered, concentrated under reduced presume to yield a residue which was purified by flash chromatography to afford 3-(3-{3-t-butyl-5-[3-(4-methoxy-phenyl)-ureido]-pyrazol-1-yl}-phenyl)-propionic acid ethyl ester (210 mg, 45%). ¹H-NMR (CD₃OD): 7.46 (t, J=7.6 Hz, 1H), 7.38 (s, 1H), 7.34 (d, J=7.6 Hz, 2H), 7.24 (d, J=8.4 Hz, 2H), 6.84 (d, J=8.4 Hz, 2H), 6.38 (s, 1H), 4.09 (q, J=7.2 Hz, 2H), 3.75 (s, 3H), 3.00 (t, J=7.6 Hz, 2H), 2.68 (t, J=7.6 Hz, 2H), 1.33 (s, 9H), 1.20 (t, J=7.6 Hz, 3H); MS (ESI) m/z: 465 (M+H⁺).

### Example 148

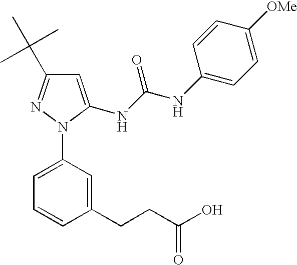

Utilizing the same synthetic procedure as for Example 61 and starting with Example 147, 3-(3-{3-t-butyl-5-[3-(4-methoxy-phenyl)-ureido]-pyrazol-1-yl}-phenyl)-propionic acid is synthesized.

### Example 149

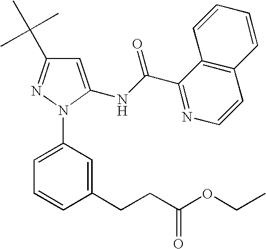

A solution of isoquinoline-1-carboxylic acid (346 mg, 2.0 mmol), Example Z (315 mg, 1.0 mmol), EDCI (394 mg, 2.0 mmol), HOBt (270 mg, 2.0 mmol), and NMM (1.0 mL) in DMF (10 mL) was stirred at RT overnight. After quenching with water (100 mL), the reaction mixture was extracted with ethyl acetate (3×100 mL). The combined organic extracts were washed with brine, dried (NaSO₄), filtered and concentrated under reduced pressure to yield a residue which was purified by flash chromatography to afford 3-(3-{3-t-butyl-5-[(isoquinoline-1-carbonyl)-amino]-pyrazol-1-yl}-phenyl)-propionic acid ethyl ester, (380 mg, 80%). ¹H-NMR (DMSO-d₆): 8.83 (d, J=8.4 Hz, 1H), 8.85 (d, J=5.2 Hz, 1H), 8.09 (s, 1H), 8.07 (d, J=8.4 Hz, 1H), 7.82 (t, J=8.0 Hz, 1H), 7.72 (t, J=8.0 Hz, 1H), 7.52 (s, 1H), 7.44 (d, J=8.0 Hz, 1H), 7.39 (t, J=5.2 Hz, 1H), 7.22 (d, J=8.0 Hz, 1H), 6.57 (s, 1H), 3.98 (q, J=7.2 Hz, 2H), 2.84 (t, J=7.6 Hz, 2H), 2.57 (t, J=7.6 Hz, 2H), 1.32 (s, 9H), 1.10 (t, J=7.6 Hz, 1H); MS (ESI) m/z: 471 (M+H⁺).

### Example 150

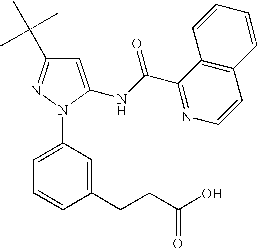

A solution of Example 149u (47 mg, 0.1 mmol) and 2N LiOH (3 mL) in MeOH (3 mL) was stirred at RT overnight. The reaction mixture was neutralized to pH=4, extracted with ethyl acetate (3×20 mL), and the combined organic extracts were washed with brine, dried (NaSO₄) and filtered. The filtrate was concentrated to afford 3-(3-{3-t-butyl-5-[(isoquinoline-1-carbonyl)-amino]-pyrazol-1-yl}-phenyl)-propionic acid, (39 mg, 87%). ¹H-NMR (DMSO-d6): 10.77 (s, 1H), 9.68 (d, J=7.6 Hz, 1H), 8.44 (d, J=5.2 Hz, 1H), 7.89-7.44 (m, 2H), 7.78-7.74 (m, 2H), 7.49-7.47 (m, 3H), 7.30-7.27 (m, 3H), 6.95 (s, 1H), 3.05 (t, J=7.2 Hz, 2H), 2.75 (t, J=7.6 Hz, 2H), 1.42 (s, 9H); MS (ESI) m/z: 443 (M+H⁺).

### Example 151

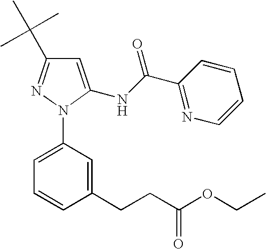

A solution of pyridine-2-carboxylic acid (246 mg, 2.0 mmol), Example Z (315 mg, 1.0 mmol), EDCI (394 mg, 2.0 mmol), HOBt (270 mg, 2.0 mmol), NMM (1.0 mL) in DMF (10 mL) was stirred at RT overnight. After quenching with water (100 mL), the reaction mixture was extracted with ethyl acetate (3×100 mL). The combined organic extracts were washed with brine, dried (NaSO₄), filtered and concentrated under reduced pressure to yield a residue which was purified by flash chromatography to afford 3-(3-{3-t-butyl-5-[(pyridine-2-carbonyl)-amino]-pyrazol-1-yl}-phenyl)-propionic acid ethyl ester (300 mg, 70%). ¹H-NMR (CDCL₃): 8.53 (d, J=4.4 Hz, 1H), 8.26 (d, J=7.2 Hz, 1H), 7.90 (t, J=8.0 Hz, 1H), 7.48-7.43 (m, 4H), 7.27 (s, 1H), 6.87 (s, 1H), 4.13 (q, J=7.2 Hz, 2H), 3.04 (t, J=7.6 Hz, 2H), 2.71 (t, J=7.6 Hz, 2H), 1.39 (s, 9H), 1.24 (t, J=7.2 Hz, 3H); MS (ESI) m/z: 421 (M+H⁺).

### Example 152

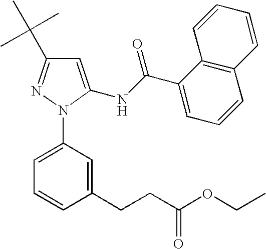

A solution of Example Z (315 mg, 1.0 mmol) and Barton's base (0.5 mL) in anhydrous CH₂Cl₂ (5 mL) under N₂ was stirred at RT for 30 min, and then added to a solution of naphthalene-1-carbonyl fluoride (348 mg, 0.2 mmol) in anhydrous CH₂Cl₂ (5 mL). The resulting mixture was stirred at RT overnight. After quenching with water (100 mL), the reaction mixture was extracted with ethyl acetate (3×100 mL). The combined organic extracts were washed with brine, dried (NaSO₄), filtered and concentrated under reduced pressure to yield a residue which was purified by flash chromatography to afford 3-(3-{3-t-butyl-5-[(naphthalene-1-carbonyl)-amino]-pyrazol-1-yl}-phenyl)-propionic acid ethyl ester, (350 mg, 74%). ¹H-NMR (CDCL₃): 8.29 (d, J=8.0 Hz, 1H), 7.98 (d, J=7.2 Hz, 2H), 7.89 (d, J=7.2 Hz, 1H), 7.62-7.57 (m, 3H), 7.49-7.28 (m, 4H), 7.03 (s, 1H), 3.94 (q, J=7.2 Hz, 2H), 2.96 (t, J=7.2 Hz, 2H), 2.58 (t, J=7.2 Hz, 2H), 1.45 (s, 9H), 1.13 (t, J=7.2 Hz, 3H); MS (ESI) m/z: 470 (M+H⁺).

### Example 153

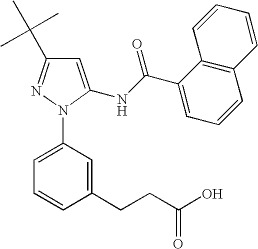

A solution of Example 152 (47 mg, 0.1 mmol) and 2N LiOH (3 mL) in MeOH (3 mL) was stirred at RT overnight. The reaction mixture was neutralized to pH=4, and extracted with ethyl acetate (3×20 mL). The combined organic extracts were washed with brine, and dried (NaSO₄) and filtered. The filtrate was concentrated to afford 3-(3-{3-t-butyl-5-[(isoquinoline-1-carbonyl)-amino]-pyrazol-1-yl}-phenyl)-propionic acid, (38 mg, 86%). ¹H NMR (DMSO-d6): 7.99 (d, J=8.0 Hz, 1H), 7.90 (m, 2H), 7.62 (m, 1H), 7.54-7.42 (m, 6H), 7.35 (m, 1H), 6.54 (s, 1H), 2.94 (t, J=7.6 Hz, 2H), 2.57 (t, J=7.2 Hz, 2H), 1.38 (s, 9H); MS (ESI) m/z: 443 (M+H⁺).

### Example 154

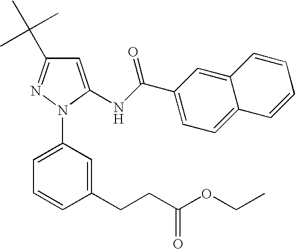

A solution of naphthalene-2-carboxylic acid (344 mg, 2.0 mmol) in SOCl₂ (10 mL) was heated to reflux for 2 h. After concentration under reduced pressure, the residue was dissolved into CH₂Cl₂ (5 mL) and was dropped into a solution of Example Z (315 mg, 1.0 mmol) in CH₂Cl₂ (10 mL) at 0° C., and was then stirred at RT overnight. After quenching with water (50 mL), the reaction mixture was extracted with CH₂Cl₂ (3×100 mL). The combined organic extracts were washed with brine, dried (NaSO₄), filtered and concentrated under reduced pressure to yield a residue which was purified by flash chromatography to afford 3-(3-{3-t-butyl-5-[(naphthalene-2-carbonyl)-amino]-pyrazol-1-yl}-phenyl)-propionic acid ethyl ester (180 mg, 38%). ¹H-NMR (CDCL₃): 8.24 (s, 1H), 8.21 (s, 1H), 7.91 (d, J=8.4 Hz, 2H), 7.88 (d, J=8.4 Hz, 1H), 7.73 (d, J=8.4 Hz, 1H), 7.63-7.49 (m, 3H), 7.45-7.26 (m, 3H), 6.94 (s, 1H), 4.02 (q, J=7.2 Hz, 2H), 3.04 (t, J=7.6 Hz, 2H), 2.67 (t, J=7.6 Hz, 2H), 1.43 (s, 9H), 1.17 (t, J=7.2 Hz, 3H); MS (ESI) m/z: 470 (M+H⁺).

### Example 155

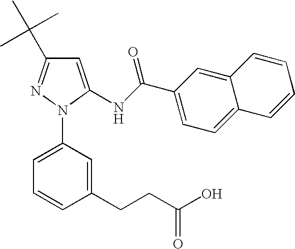

A solution of Example 154 (47 mg, 0.1 mmol) and 2N LiOH (3 mL) in MeOH (3 mL) was stirred at RT overnight. The reaction mixture was neutralized to pH=4, and extracted with ethyl acetate (3×20 mL). The combined organic extracts were washed with brine, and dried (NaSO₄) and filtered. The filtrate was concentrated to afford 3-(3-{3-t-butyl-5-[(isoquinoline-2-carbonyl)-amino]-pyrazol-1-yl}-phenyl)-propionic acid, (37 mg, 84%). ¹H-NMR (CDCL3): 8.25 (s, 1H), 8.18 (s, 1H), 7.91-7.86 (m, 3H), 7.75 (d, J=8.0 Hz, 1H), 7.59-7.55 (m, 2H), 7.48-7.39 (m, 3H), 7.28 (s, 1H), 6.81 (s, 1H), 3.02 (t, J=7.6 Hz, 2H), 2.69 (t, J=7.6 Hz, 2H), 1.42 (s, 9H); MS (ESI) m/z: 442 (M+H⁺).

### Example 156

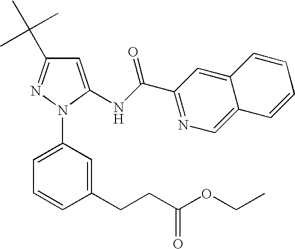

A solution of isoquinoline-3-carboxylic acid (346 mg, 2.0 mmol), example Z (315 mg, 1.0 mmol), EDCI (394 mg, 2.0 mmol), HOBt (270 mg, 2.0 mmol), and NMM (1.0 mL) in DMF (10 mL) was stirred at RT overnight. After quenching with water (50 mL), the reaction mixture was extracted with ethyl acetate (3×100 mL). The combined organic extracts were washed with brine, dried (NaSO₄) and filtered. After concentrated under reduced pressure, the residue was purified by flash chromatography to afford 3-(3-{3-t-butyl-5-[(isoquinoline-3-carbonyl)-amino]-pyrazol-1-yl}-phenyl)-propionic acid ethyl ester (250 mg, 54%). ¹H-NMR (CD₃OD): 9.24 (s, 1H), 8.63 (s, 1H), 8.17 (d, J=8.0 Hz, 1H), 8.11 (d, J=8.0 Hz, 1H), 7.88 (t, J=7.6 Hz, 1H), 7.81 (t, J=7.6 Hz, 1H), 7.50 (s, 1H), 7.48 (d, J=7.6 Hz, 1H), 7.54 (d, J=7.6 Hz, 2H), 7.36 (d, J=7.6 Hz, 1H), 6.75 (s, 1H), 4.04 (q, J=7.6 Hz, 2H), 3.01 (t, J=7.6 Hz, 2H), 2.69 (t, J=7.6 Hz, 2H), 1.39 (s, 9H), 1.14 (t, J=7.6 Hz, 3H); MS (ESI) m/z: 471 (M+H⁺).

### Example 157

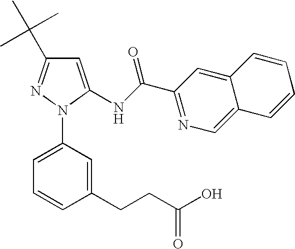

A solution of Example 156 (47 mg, 0.1 mmol) and 2N LiOH (3 mL) in MeOH (3 mL) was stirred at RT overnight. The reaction mixture was neutralized to pH=4, and extracted with ethyl acetate (3×20 mL). The combined organic extracts were washed with brine, and dried (NaSO₄) and filtered. The filtrate was concentrated to afford 3-(3-{3-t-butyl-5-[(isoquinoline-3-carbonyl)-amino]-pyrazol-1-yl}-phenyl)-propionic acid, (39 mg, 88%). ¹H NMR (CDCL3): 10.49 (s, 1H), 9.16 (s, 1H), 8.69 (s, 1H), 8.03 (d, J=7.6 Hz, 2H), 7.81 (t, J=7.2 Hz, 1H), 7.73 (t, J=7.2 Hz, 1H), 7.48-7.39 (m, 3H), 7.28 (br s, 1H), 6.94 (s, 1H), 3.02 (t, J=7.6 Hz, 2H), 2.79 (t, J=7.6 Hz, 2H), 1.42 (s, 9H); MS (ESI) m/z: 442 (M+H⁺).

### Example 158

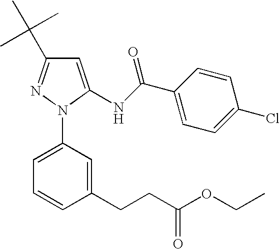

A solution of 4-chlorobenzoic acid (312 mg, 2.0 mmol) in SOCl₂ (10 mL) was heated to reflux for 2 h. After removal of the solvent, the residue was dissolved into CH₂Cl₂ (5 mL) and was dropped into a solution of Example Z (315 mg, 1.0 mmol) in CH₂Cl₂ (10 mL) at 0° C., was then stirred at RT overnight. After quenching with water (50 mL), the reaction mixture was extracted with CH₂Cl₂ (3×100 mL). The combined organic extracts were washed with brine, dried (NaSO₄) and filtered. After concentrated under reduced pressure, the residue was purified by flash chromatography to afford 3-{3-[3-t-butyl-5-(4-chloro-benzoylamino)-pyrazol-1-yl]-phenyl}-propionic acid ethyl ester (290 mg, 64%). ¹H-NMR (CDCL₃): 8.02 (s, 1H), 7.67 (d, J=8.4 Hz, 2H), 7.46 (t, J=7.6 Hz, 1H), 7.44 (d, J=8.4 Hz, 2H), 7.36 (t, J=8.4 Hz, 3H), 6.87 (s, 1H), 4.06 (q, J=7.6 Hz, 2H), 3.02 (t, J=7.6 Hz, 2H), 2.67 (t, J=7.6 Hz, 2H), 1.40 (s, 9H), 1.12 (t, J=7.6 Hz, 3H); MS (ESI) m/z: 454 (M+H⁺).

### Example 159

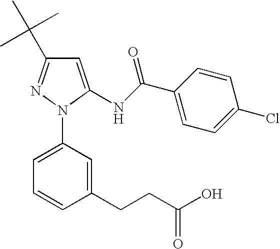

A solution of Example 158 (45 mg, 0.1 mol) and 2N LiOH (3 mL) in MeOH (3 mL) was stirred at RT overnight. The reaction mixture was neutralized to pH=4, and extracted with ethyl acetate (3×20 mL). The combined organic extracts were washed with brine, and dried (NaSO₄) and filtered. The filtrate was concentrated to afford 3-{3-[3-t-butyl-5-(4-chloro-benzoylamino)-pyrazol-1-yl]-phenyl}-propionic acid, (38.5 mg, 87%). ¹H NMR (DMSO-d6): 10.38 (s, 1H), 7.85 (d, J=8.4 Hz, 1H), 7.56 (d, J=8.4 Hz, 2H), 7.39 (s, 1H), 7.32 (d, J=4.8 Hz, 2H), 7.15 (t, J=4.8 Hz, 1H), 6.38 (s, 1H), 2.80 (t, J=7.6 Hz, 2H), 2.44 (t, J=7.2 Hz, 2H), 1.29 (s, 9H); MS (ESI) m/z: 426 (M+H⁺).

### Example AA

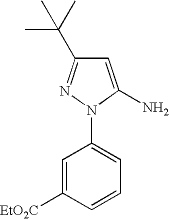

To a solution of m-aminobenzoic acid (200.0 g, 1.46 mmol) in concentrated HCl (200 mL) was added an aqueous solution (250 mL) of NaNO₂ (102 g, 1.46 mmol) at 0° C. and the reaction mixture was stirred for 1 h. A solution of SnCl₂.2H₂O (662 g, 2.92 mmol) in concentrated HCl (2000 mL) was then added at 0° C. The reaction solution was stirred for an additional 2 h at RT. The precipitate was filtered and washed with ethanol and ether to give 3-hydrazino-benzoic acid hydrochloride as a white solid, which was used for the next reaction without further purification. ¹H NMR (DMSO-d₆): 10.85 (s, 3H), 8.46 (s, 1H), 7.53 (s, 1H), 7.48 (d, J=7.6 Hz, 1H), 7.37 (m, J=7.6 Hz, 1H), 7.21 (d, J=7.6 Hz, 1H).

A mixture of 3-hydrazino-benzoic acid hydrochloride (200 g, 1.06 mol) and 4,4-dimethyl-3-oxo-pentanenitrile (146 g, 1.167 mol) in ethanol (2 L) was heated to reflux overnight. The reaction solution was evaporated under reduced pressure. The residue was purified by column chromatography to give 3-(5-amino-3-t-butyl-pyrazol-1-yl)-benzoic acid ethyl ester (116 g, 40%) as a white solid together with 3-(5-amino-3-t-butyl-pyrazol-1-yl)-benzoic acid (93 g, 36%). 3-(5-amino-3-t-butyl-pyrazol-1-yl)-benzoic acid and ethyl ester: ¹H NMR (DMSO-d₆): 8.09 (s, 1H), 8.05 (brd, J=8.0 Hz, 1H), 7.87 (br d, J=8.0 Hz, 1H), 7.71 (t, J=8.0 Hz, 1H), 5.64 (s, 1H), 4.35 (q, J=7.2 Hz, 2H), 1.34 (t, J=7.2 Hz, 3H), 1.28 (s, 9H).

### Example BB

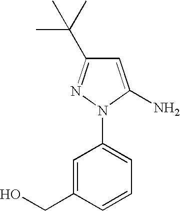

To a stirred solution of Example AA (19.5 g, 68.0 mmol) in THF (200 mL) was added LiAlH₄ powder (5.30 g, 0.136 mol) at −10° C. under N₂. The mixture was stirred for 2 h at RT and excess LiAlH₄ was destroyed by slow addition of ice. The reaction mixture was acidified to pH=7 with diluted HCl, the solution concentrated under reduced pressure, and the residue was extracted with ethyl acetate. The combined organic extracts were concentrated to give [3-(5-amino-3-t-butyl-pyrazol-1-yl)-phenyl]-methanol (16.35 g, 98%) as a white powder. ¹H NMR (DMSO-d6): 9.19 (s, 1H), 9.04 (s, 1H), 8.80 (s, 1H), 8.26-7.35 (m, 1H), 6.41 (s, 1H), 4.60 (s, 2H), 1.28 (s, 9H); MS (ESI) m/z: 415 (M+H⁺).

### Example CC

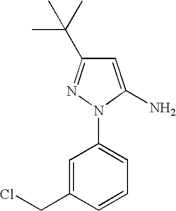

A solution of Example BB (13.8 g, 56.00 mmol) and SOCl₂ (8.27 mL, 0.11 mol) in THF (200 mL) was refluxed for 3 h and concentrated under reduced pressure to yield 5-t-butyl-2-(3-chloromethyl-phenyl)-2H-pyrazol-3-ylamine (14.5 g, 98%) as white powder which was used without fitter purification. ¹H NMR (DMSO-d6), δ 7.62 (s, 1H), 7.53 (d, J=8.0 Hz, 1H), 7.43 (t, J=8.0 Hz, 1H), 7.31 (d, J=7.2 Hz, 1H), 5.38 (s, 1H), 5.23 (br s, 2H), 4.80 (s, 2H), 1.19 (s, 9H). MS (ESI) m/z: 264 (M+H⁺).

### Example DD

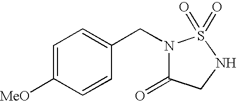

To a stirred solution of chlorosulfonyl isocyanate (1.43 g, 10.0 mmol) in CH₂Cl₂ (20 mL) at 0° C. was added 2-methyl-propan-2-ol (0.74 g, 10.0 mmol) at such a rate that mmol) in portions under N₂ atmosphere at RT. The mixture was stirred overnight then quenched with HCl/methanol (2 M). After the solvent was removed in vacuo, the residue was washed with water to afford 2-(4-methoxy-benzyl)-1,1-dioxo-1λ⁶-[1,2,5]thiadiazolidin-3-one (480 mg, 54%). ¹H-NMR(CDCl₃): δ 7.36 (d, J=8.4 Hz, 2H), 6.87 (d, J=8.8 Hz, 2H), 4.87 (m, 1H), 4.68 (s, 2H), 4.03 (d, J=7.2 Hz, 2H), 3.80 (s, 3H).

### Example EE

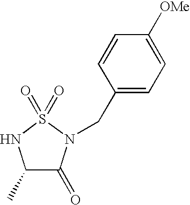

To a stirred solution of chlorosulfonyl isocyanate (1.43 g, 10.0 mmol) in CH₂Cl₂ (20 mL) at 0° C. was added benzyl alcohol (1.08 g, 10.0 mmol) at such a rate that the reaction solution temperature did not rise above 5° C. After stirring for 1.5 h, a solution of L-alanine methyl ester (1.45 g, 12.0 mmol) and Et₃N (3.2 mL, 25.0 mmol) in CH₂Cl₂ (20 mL) was added at such a rate that the reaction temperature didn't rise above 5° C. When the addition was completed, the reaction solution was allowed to warm up to RT and stirred overnight. The reaction mixture was poured into 10% HCl, extracted with CH₂Cl₂, the organic extracts washed with saturated NaCl, dried (Mg₂SO₄), and filtered. After removal of the solvent, the crude product was recrystallized in PE/EA (10:1) to afford the desired product (2.5 g, 79%), which was used directly in the next step. ¹H-NMR (DMSO): δ 11.31 (s, 1H), 8.43 (d, J=8.0 Hz, 1H), 7.37-7.32 (m, 5H), 5.11 (s, 2H), 4.03 (m, 1H), 3.57 (s, 3H), 1.23 (d, J=7.2 Hz, 3H).

A mixture of material from the previous reaction (2.5 g, 12 mmol) and Pd/C (10%, 250 mg) in methanol was stirred for 4 h at 50° C. under H₂ atmosphere (55 psi). After the catalyst was removed by suction, the filtrate was evaporated to afford the desired compound (1.37 g, 92%) as a white solid, which was used directly in the next step. ¹H-NMR (CDCl₃): δ 5.51 (d, J=5.6 Hz, 1H), 4.94 (br, 2H), 4.18 (m, 1H), 3.78 (s, 3H), 1.46 (d, J=7.2 Hz, 3H).

To a solution of 2.0 N of NaOMe in methanol (20 mL) was added a solution of compound form the previous reaction (1.2 g, 6.1 mmol) in methanol and the resulting mixture was heated to reflux overnight. After cooling down, a solution of HCl in methanol was added to acidify to pH 7. The resulted salt was filtered off and the filtrate was evaporated to dryness to afford a light yellow solid which was used directly in the next step (600 mg, 66%). ¹H-NMR (DMSO-d₆): δ 6.04 (d, J=4.8 Hz, 1H), 3.60 (m, 1H), 1.11 (d, J=7.2 Hz, 3H).

A mixture of compound from the previous step (500 mg, 3.33 mmol) and 1-chloromethyl-4-methoxybenzene (156 mg, 1.0 mmol) in acetonitrile was heated to reflux overnight together with K₂CO₃ (207 mg, 1.5 mmol) and KI (250 mg, 1.5 mmol) under N₂ atmosphere. After cooling, the salt was filtered off and the filtrate was purified by column to afford 2-(4-methoxybenzyl)-(S)-4-methyl-1,1-dioxo-1λ⁶-[1,2,5]thiadiazolidin-3-one as a white solid (200 mg), which was used without further purification.

### Example 160

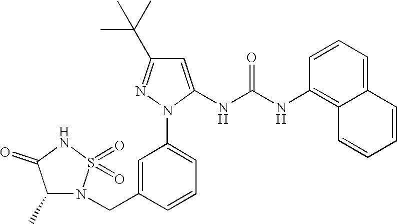

To a solution of Example EE (100 mg, 0.37 mmol) in anhydrous DMF (3 mL) was added NaH (18 mg, 0.44 mmol) at 0° C. After stirring for 0.5 h at 0° C., a solution of Example E (160 mg, 0.37 mmol) in anhydrous DMF (3 mL) was added to the reaction mixture, which was stirred overnight at RT and subsequently concentrated under reduced pressure to yield a crude solid which was used without further purification.

A solution of the crude material from the previous reaction (60 mg, 0.090 mmol) in trifluoroacetic acid (3 mL) was stirred at 50° C. for 4 h. After the solvent was removed, the residue was purified by preparative HPLC to afford 1-{5-t-butyl-2-[3-((S)-3-methyl-1,1,4-trioxo-1λ⁶-[1,2,5]thiadiazolidin-2-ylmethyl)-phenyl]-2H-pyrazol-3-yl}-3-naphthalen-1-yl-urea as white power (45 mg). ¹H NMR (DMSO-d₆): 9.04 (s, 1H), 8.87 (s, 1H), 8.02 (d, J=8.0 Hz, 1H), 7.89 (d, J=7.2 Hz, 2H), 7.62 (d, J=8.0 Hz, 2H), 7.41-7.52 (m, 6H), 6.40 (s, 1H), 4.31-4.49 (dd, J=8.0 Hz, 2H), 4.03 (q, J=6.8 Hz, 1H), 1.27 (s, 9H), 1.17 (d, J=8.0 Hz, 3H). MS (ESI) m/z: 547 (M+H⁺).

### Example FF

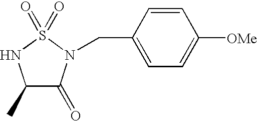

2-(4-methoxy-benzyl)-(R)-4-methyl-1,1-dioxo-1λ⁶-[1,2,5]thiadiazolidin-3-one was prepared from D-alanine ethyl ester using the same procedure as Example EE.

### Example 161

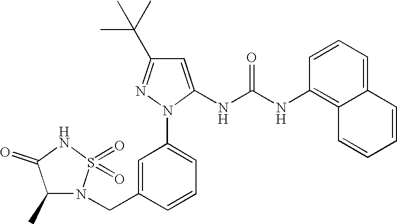

To a solution of Example FF (60 mg, 0.22 mmol) in anhydrous DMF (2 mL) was added NaH (11 mg, 0.27 mmol) at 0° C. After stirring for 0.5 h at 0° C., a solution of Example D (100 mg, 0.22 mmol) in anhydrous DMF (2 mL) was added to the reaction mixture, which was stirred overnight at RT. The crude reaction mixture was concentrated under reduced pressure and the residue by purified through preparative HPLC to yield 1-(5-t-butyl-2-{3-[5-(4-methoxy-benzyl)-(R)-3-methyl-1,1,4-trioxo-1) 1λ⁶-[1,2,5]-thiadiazolidin-2-ylmethyl}-phenyl}-2H-pyrazol-3-yl)-3-naphthalene-1-yl-urea (20 mg). ¹H NMR (DMSO-d₆): 8.98 (s, 1H), 8.81 (s, 1H), 8.00 (d, J=8.0 Hz, 1H), 7.90 (d, J=7.2 Hz,2H), 7.62(s, 2H), 7.51-7.55 (m, 6H), 7.44 (d, J=7.6 Hz, 2H), 7.22 (d, J=8.8 Hz, 2H), 6.86 (d, J=8.8 Hz, 2H), 6.40 (s, 1H), 4.57-4.62 (dd, J=8.0 Hz, 4H), 4.53 (q, J=7.6 Hz, 1H), 3.71 (s, 3H), 1.30 (d, J=8.0 Hz, 3H), 1.27 (s, 9H). MS (ESI) m/z: 653 (M+H⁺).

A solution of 1-(5-t-Butyl-2-{3-[5-(4-methoxy-benzyl)-(R)-3-methyl-1,1,4-trioxo-1λ⁶-[1,2,5]-thiadiazolidin-2-ylmethyl]-phenyl}-2H-pyrazol-3-yl)-3-naphthalen-1-yl-urea (20 mg, 0.030 mmol) in trifluoroacetic acid (2 mL) was stirred at 50° C. for 4 h. After the solvent was removed, the residue was purified by preparative-HPLC to afford 1-{5-t-butyl-2-[3-((R)-3-methyl-1,1,4-trioxo-1λ⁶-[1,2,5]thiadiazolidin-2-ylmethyl)-phenyl]-2H-pyrazol-3-yl}-3-naphthalen-1-yl-urea as a white power (6 mg). ¹H NMR (DMSO-d₆): 8.99 (s, 1H), 8.80 (s, 1H), 8.00 (d, J=7.2 Hz, 1H), 7.90 (d, J=7.2 Hz, 2H), 7.60-7.64 (m, 2H), 7.44-7.54 (m, 7H), 6.41 (s, 1H), 4.31-4.49 (dd, J=8.0 Hz, 2H), 4.03 (q, J=7.6 Hz, 1H), 1.27 (s, 9H), 1.19 (d, J=8.0 Hz, 3H). MS (ESI) m/z: 533 (M+H⁺).

### Example 162

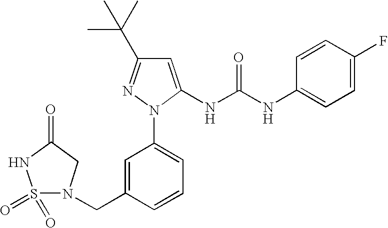

To a solution of Example CC (0.263 g, 1.0 mmol) in THF (2.0 mL) was added a solution of 1-fluoro-4-isocyanato-benzene (0.114 mL, 1.10 mmol) in THF (5.0 mL) at 0° C. The mixture was stirred at RT for 1 h then heated until all solids were dissolved. The mixture was stirred at RT for 3 h and poured into water (20 mL). The resulting precipitate was filtered, washed with diluted HCl and H₂O, dried under reduced pressure to yield 1-[5-t-butyl-2-(3-chloromethyl-phenyl)-2H-pyrazol-3-yl]-3-(4-fluoro-phenyl)-urea (400 mg) as a white power. ¹H NMR (DMSO-d₆): 8.99 (s, 1H), 8.38 (s, 1H), 7.59 (s, 1H), 7.44-7.51 (m, 3H), 7.38-7.40 (m, 2H), 7.08 (t, J=8.8 Hz, 2H), 6.34 (s, 1H), 4.83 (s, 2H), 1.26 (s, 9H). MS (ESI) m/z: 401 (M+H⁺).

To a solution of 2-(4-methoxy-benzyl)-1,1-dioxo-1λ⁶-[1,2,5]thiadiazolidin-3-one (64 mg, 0.25 mmol) in anhydrous DMF (2 mL) was added NaH (11 mg, 0.27 mmol) at 0° C. After stirred for 0.5 h at 0° C., a solution of 1-[5-t-butyl-2-(3-chloromethyl-phenyl)-2H-pyrazol-3-yl]-3-(4-fluoro-phenyl)-urea from the previous reaction (100 mg, 0.25 mmol) in anhydrous DMF (2 mL) was added to the reaction mixture, then was stirred overnight at RT. The crude was purified through prepared-HPLC to yield 1-(5-t-butyl-2-{3-[5-(4-methoxy-benzyl)-1,1,4-trioxo-1λ⁶-[1,2,5]thiadiazolidin-2-ylmethyl]-phenyl}-2H-pyrazol-3-yl)-3-(4-fluoro-phenyl)-urea (45 mg). ¹H NMR (DMSO-d₆): 8.95 (s, 1H), 8.37 (s, 1H), 7.50-7.54 (m, 3H), 7.36-7.41 (m, 3H), 7.25 (d, J=8.8 Hz, 2H), 7.07 (t, J=8.8 Hz, 2H), 6.87 (d, J=8.4 Hz, 2H), 6.35 (s, 1H), 4.64 (s, 2H), 4.47 (s, 2H), 4.19 (s, 2H), 3.75 (s, 3H), 1.26 (s, 9H). MS (ESI) m/z: 515 (M+H⁺).

A solution of 1-(5-t-butyl-2-{3-[5-(4-methoxy-benzyl)-1,1,4-trioxo-1λ⁶-[1,2,5]thiadia-zolidin-2-ylmethyl]-phenyl}-2H-pyrazol-3-yl)-3-(4-fluoro-phenyl)-urea (40 mg, 0.060 mmol) in trifluoroacetic acid (3 mL) was stirred at 50° C. for 4 h. After the solvent was removed, the residue was purified by preparative HPLC to afford 1-{5-t-butyl-2-[3-(3-(R)-methyl-1,1,4-trioxo-1λ⁶-[1,2,5]thiadiazolidin-2-ylmethyl)-phenyl]-2H-pyrazol-3-yl}-3-naphthalen-1-yl-urea as a white power (12 mg). ¹H NMR (DMSO-d₆): 8.98 (s, 1H), 8.39 (s, 1H), 7.37-7.51 (m, 6H), 7.07 (t, J=8.8 Hz, 2H), 6.35 (s, 1H), 4.21 (s, 2H), 3.88 (s, 2H), 1.26 (s, 9H). MS (ESI) m/z: 501 (M+H⁺).

### Example GG

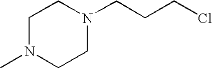

To a stirred suspension of K₂CO₃ (5.5 g, 40 mmol) and 1-bromo-3-chloro-propane (3.78 g, 24 mmol) in acetonitrile (110 mL) was added a solution of N-methyl piperazine (2.0 g, 20 mmol) in acetonitrile (10 mL) dropwise at RT. After the addition was completed, the reaction mixture was stirred for 3 h then filtered. The filtrate was concentrated and dissolved in CH₂Cl₂, washed with brine, dried (NaSO₄) and filtered. After removal of the solvent, the residue was dissolved in ether. To the above solution was added the solution of HCl and filtered to afford the desired product (2.3 g, 65.7%). ¹H NMR (D₂O): 3.61 (t, J=6.0 Hz, 2H), 3.59 (br, 8H), 3.31 (t, J=8.0 Hz, 2H), 2.92 (s, 3H), 2.15 (m, 2H).

### Example 163

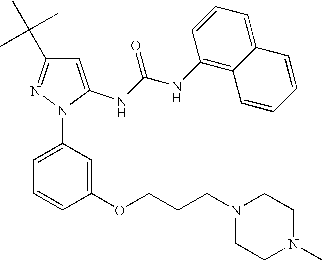

To a solution of Example 41 (100 mg, 0.25 mmol) in acetonitrile (10 mL) was added Example GG (75 mg, 0.30 mmol) and K₂CO₃ (172 mg, 1.25 mmol). The resulting mixture was stirred at 45° C. for 3 h before filtered. After the filtrate was concentrated, the residue was purified by preparative TLC to afford 1-(5-t-Butyl-2-{3-{3-(4-methyl-piperazin-1-yl)-propoxy]-phenyl}-2H-pyrazol-3-yl)-3-naphthalen-1-yl-urea (31 mg, 23%). ¹H-NMR (CD₃OD): 7.93 (m, 1H), 7.88 (m, 1H), 7.71 (d, J=8.4 Hz, 1H), 7.66 (d, J=7.6 Hz, 1H), 7.43-7.50 (m, 4H), 7.14 (m, 2H), 7.05 (m, 1H), 6.43 (s, 1H), 4.10 (t, J=6.0 Hz, 2H), 3.09-3.15 (br, 4H), 2.74-2.86(br, 6H), 2.72 (s, 3H), 1.99 (t, J=6.8 Hz, 2H), 1.35 (s, 9H). MS (ESI) m/z: 541 (M+H⁺).

### Example HH

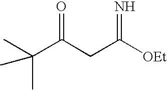

Intermediate HH was synthesized according to literature procedures starting from 4,4-dimethyl-3-oxo-pentanenitrile (10 mmole) in absolute ethanol and HCl in quantitative afford.

### Example II

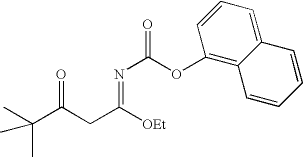

Intermediate HH (5 g, 0.0241 mol) is added to pyridine (5 mL) in CH₂Cl₂ (25 mL) and cooled in an ice bath. The suspension is stirred for 5 min and 1-napthylchloroformate is added dropwise over 5 min. The reaction mixture is stirred an additional 5 min at 0° C., and the reaction is warmed and stirred at RT for 1 h. The reaction is pour into ethyl acetate (100 mL) and water (100 ml). After shaking, the aqueous layer is removed, the organic layer washed with water, dried (MgSO₄) and concentrated to afford (Z)-naphthalen-1-yl 1-ethoxy-4,4-dimethyl-3-oxopentylidenecarbamate.

### Example 164

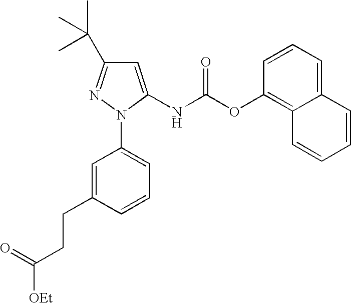

Example II (10 mmol) is dissolved in absolute EtOH (50 mL) at RT and Example Y (10.5 mmol) is added dropwise over 5 min. The reaction mixture is stirred for 30 min at RT, poured in water (100 mL) and ethyl acetate (100 mL). After shaking, the organic layer is washed with 5% HCl, water, dried (MgSO₄) and concentrated to afford 1-naphthyl 1-(3-(2-(ethoxycarbonyl)ethyl)phenyl)-3-t-butyl-1H-pyrazol-5-ylcarbamate.

### Example 165

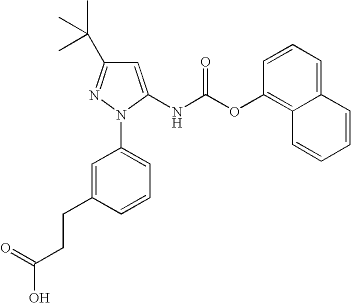

A solution of Example 164 (0.1 mmol) and 2N LiOH (3 mL) in MeOH (3 mL) is stirred at RT overnight. The reaction mixture is neutralized to pH=4, extracted with ethyl acetate (3×20 mL), the combined organic extracts are washed with brine, dried (NaSO₄) and filtered. The filtrate is concentrated to afford 1-napthyl 1-(3-(2-carboxyethylphenyl)-3-t-butyl-1H-pyrazol-5-ylcarbamate.

### Example JJ

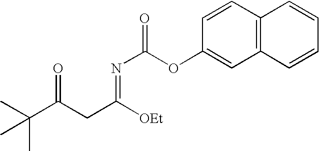

Example JJ is synthesized utilizing Example HH and 2-napthylchloroformate according to the procedure described for Example II to afford (Z)-naphthalen-2-yl-1-ethoxy-4,4-dimethyl-3-oxopentylidenecarbamate.

### Example 166

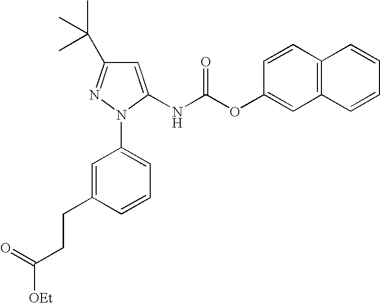

Example 166 is synthesized utilizing Example Y and Example JJ according to the procedure described for Example 79 to afford 2-naphthyl 1-(3-(2-(ethoxycarbonyl)ethyl)phenyl)-3-t-butyl-1H-pyrazol-5-ylcarbamate.

### Example 167

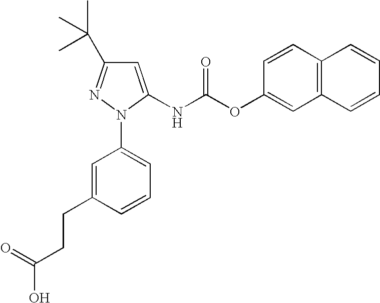

Example 167 is synthesized utilizing Example 166 according to the procedure described for Example 165 to afford 2-napthyl 1-(3-(2-carboxyethylphenyl)-3-t-butyl-1H-pyrazol-5-ylcarbamate.

### Example KK

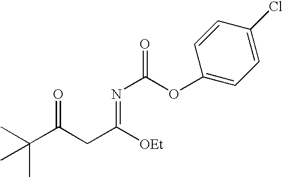

Example KK is synthesized utilizing Example HH and p-chlorophenylchloroformate according to the procedure described for Example II to afford (Z)-4-chlorophenyl 1-ethoxy-4,4-dimethyl-3-oxopentylidenecarbamate.

### Example 168

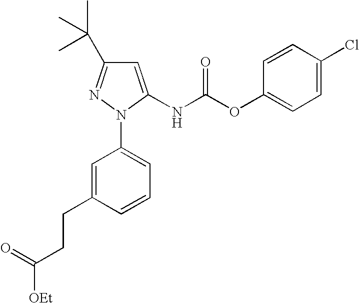

Example 168 is synthesized utilizing Example Y and Example KK according to the procedure described for Example 164 to afford 4-chlorophenyl 1-(3-(2-(ethoxycarbonyl)ethyl)phenyl)-3-t-butyl-1H-pyrazol-5-ylcarbamate.

### Example 169

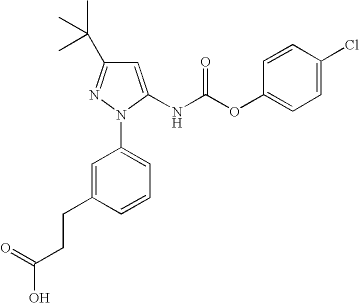

Example 169 is synthesized utilizing Example 168 according to the procedure described for Example 165 to afford 4-chlorophenyl 1-(3-(2-carboxyethylphenyl)-3-t-butyl-1H-pyrazol-5-ylcarbamate.

### Example LL

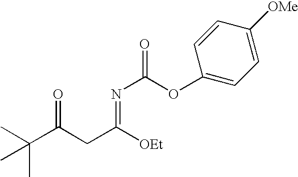

Example LL is synthesized utilizing Example HH and p-methoxyphenylchloroformate according to the procedure described for Example II to afford (Z)-4-methoxyphenyl 1-ethoxy-4,4-dimethyl-3-oxopentylidenecarbamate.

### Example 170

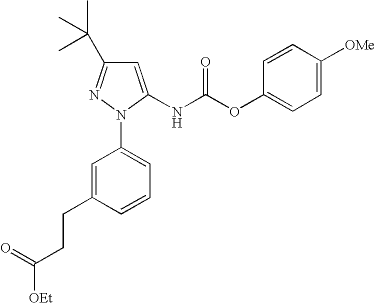

Example 170 is synthesized utilizing Example Y and Example LL according to the procedure described for Example 164 to afford 4-methoxyphenyl 1-(3-(2-(ethoxycarbonyl)ethyl)phenyl)-3-t-butyl-1H-pyrazol-5-ylcarbamate.

### Example 171

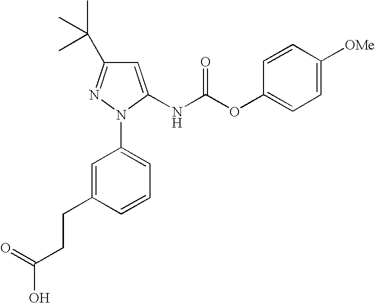

Example 171 is synthesized utilizing Example 170 according to the procedure described for Example 165 to afford 4-methoxyphenyl 1-(3-(2-carboxyethylphenyl)-3-t-butyl-1H-pyrazol-5-ylcarbamate.

### Example MM

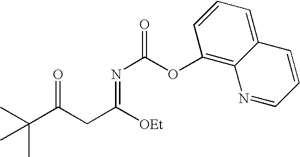

Example MM is synthesized utilizing Example HH and quinolin-8-yl-chloroformate according to the procedure described for Example II to afford (Z)-quinolin-8-yl 1-ethoxy-4,4-dimethyl-3-oxopentylidenecarbamate

### Example 172

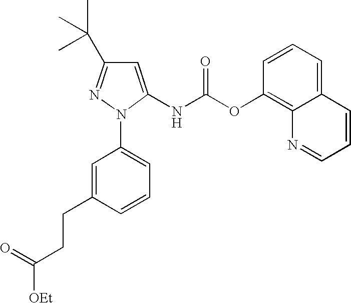

Example 172 is synthesized utilizing Example Y and Example MM according to the procedure described for Example 164 to afford quinolin-8-yl 1-(3-(2-(ethoxycarbonyl)ethyl)phenyl)-3-t-butyl-1H-pyrazol-5-ylcarbamate.

### Example 173

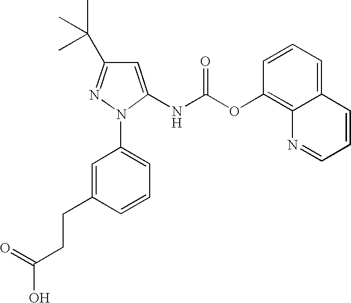

Example 173 is synthesized utilizing Example 172 according to the procedure described for Example 165 to afford quinolin-8-yl 1-(3-(2-carboxyethylphenyl)-3-t-butyl-1H-pyrazol-5-ylcarbamate.

### Example 174

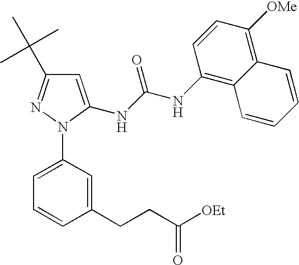

Example 174 is synthesized utilizing a mixture of 4-methoxy-1-naphthylamine and Example Z according to the procedure described for Example 147 to afford 3-(3-{3-t-butyl-5-[3-(4-methoxy-1-naphthyl)-ureido]-pyrazol-1-yl}-phenyl)-propionic acid ethyl ester.

### Example 175

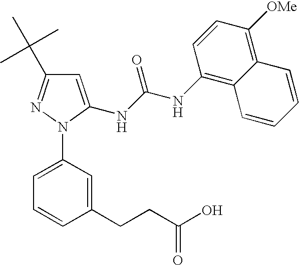

Utilizing the same synthetic procedure as for Example 146 and starting with Example 174, 3-(3-{3-t-butyl-5-[3-(4-methoxy-1-naphthyl)-ureido]-pyrazol-1-yl}-phenyl)-propionic acid is synthesized.

### Example 176

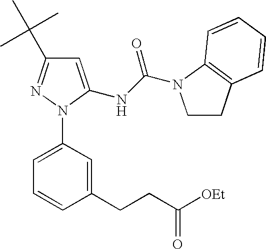

Example 176 is synthesized utilizing a mixture of indoline and Example Z according to the procedure described for Example 147 to afford ethyl 3-(3-(3-t-butyl-5-(indoline-1-carboxamido)-1H-pyrazol-1-yl)phenyl)propanoate.

### Example 177

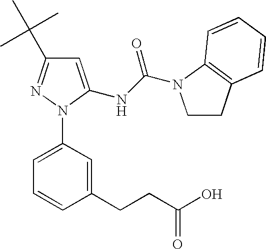

Utilizing the same synthetic procedure as for Example 146 and starting with Example 173, 3-(3-(3-t-butyl-5-(indoline-1-carboxamido)-1H-pyrazol-1-yl)phenyl) propionic acid is synthesized.

### Example NN

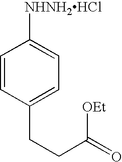

Utilizing the same synthetic procedure as for Example Y and starting with p-bromo nitrobenzene, 3-(4-hydrazino-phenyl)-propionic acid ethyl ester is synthesized.

### Example 178

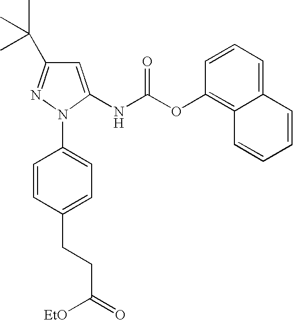

Utilizing the same synthetic procedure as for Example 164, Example II (10 mmol) and Example NN (10.5 mmol) are combined to afford 1-naphthyl 1-(4-(2-(ethoxycarbonyl)ethyl)phenyl)-3-t-butyl-1H-pyrazol-5-ylcarbamate.

### Example 179

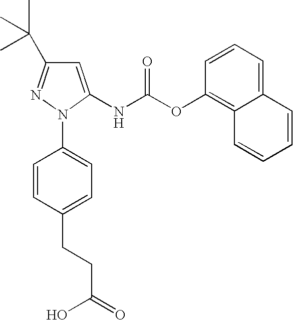

Example 179 is synthesized utilizing Example 178 according to the procedure described for Example 165 to afford 1-napthyl 1-(4-(2-carboxyethylphenyl)-3-t-butyl-1H-pyrazol-5-ylcarbamate.

### Example 180

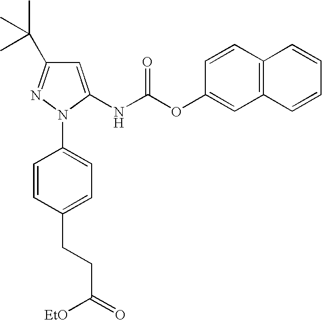

Utilizing the same synthetic procedure as for Example 164, Example JJ (10 mmol) and Example NN (10.5 mmol) are combined to afford 2-naphthyl 1-(4-(2-(ethoxycarbonyl)ethyl)phenyl)-3-t-butyl-1H-pyrazol-5-ylcarbamate.

### Example 181

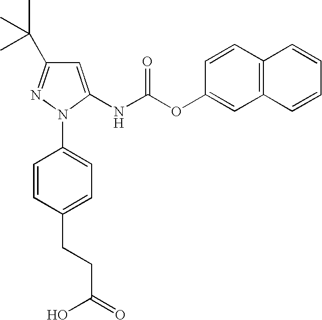

Example 181 is synthesized utilizing Example 180 according to the procedure described for Example 165 to afford 2-napthyl 1-(4-(2-carboxyethylphenyl)-3-t-butyl-1H-pyrazol-5-ylcarbamate.

### Example 182

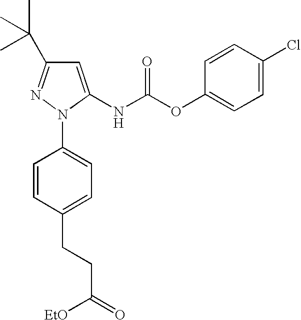

Utilizing the same synthetic procedure as for Example 164, Example KK (10 mmol) and Example NN (10.5 mmol) are combined to afford p-chlorophenyl 1-(4-(2-(ethoxycarbonyl)ethyl)phenyl)-3-t-butyl-1H-pyrazol-5-ylcarbamate.

### Example 183

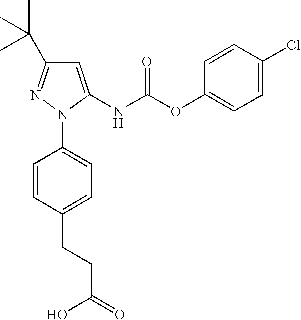

Example 183 is synthesized utilizing Example 182 according to the procedure described for Example 165 to afford 4-chlorophenyl 1-(4-(2-carboxyethylphenyl)-3-t-butyl-1H-pyrazol-5-ylcarbamate.

### Example 184

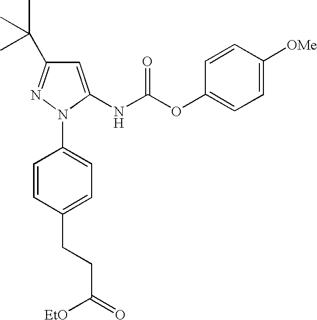

Utilizing the same synthetic procedure as for Example 164, Example LL (10 mmol) and Example NN (10.5 mmol) are combined to afford p-methoxyphenyl 1-(4-(2-(ethoxycarbonyl)ethyl)phenyl)-3-t-butyl-1H-pyrazol-5-ylcarbamate.

### Example 185

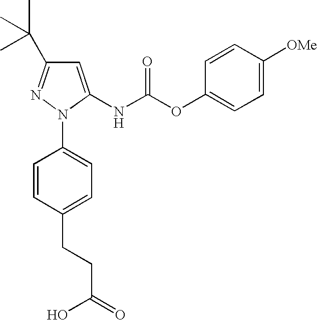

Example 185 is synthesized utilizing Example 184 according to the procedure described for Example 165 to afford p-methoxyphenyl 1-(4-(2-carboxyethylphenyl)-3-t-butyl-1H-pyrazol-5-ylcarbamate.

### Example OO

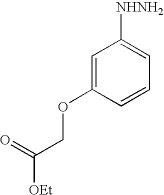

Ethyl bromoacetate is reacted with meta-nitrophenol under standard conditions to afford ethyl 2-(3-nitrophenoxy)acetate, which is elaborated to ethyl 2-(3-hydrazinophenoxy)acetate using the reduction/oxidation sequence described for Example Y.

### Example PP

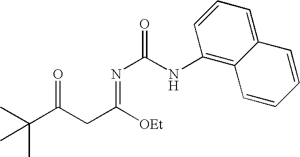

Example HH and 1-naphthylisocyanate are combined utilizing the same synthetic procedure as for Example II to afford (Z)-1-(1-ethoxy-4,4-dimethyl-3-oxopentylidene)-3-(naphthalen-1-yl)urea.

### Example 186

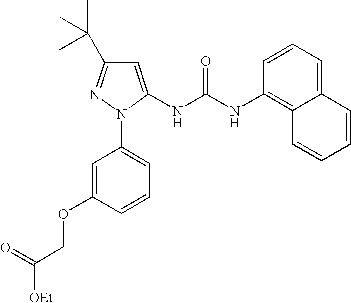

Utilizing the same synthetic procedure as for Example 164, Example PP (10 mmol) and Example OO (10.5 mmol) are combined to afford 3-(3-{3-t-butyl-5-[3-(1-naphthyl)-ureido]-pyrazol-1-yl}-phenoxy)-acetic acid ethyl ester.

### Example 187

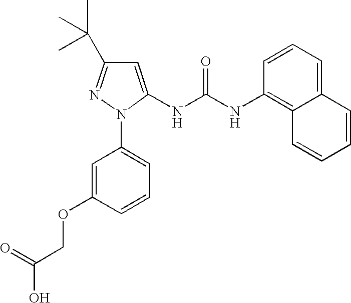

Utilizing the same synthetic procedure as for Example 146 and starting with Example 186, 3-(3-{3-t-butyl-5-[3-(1-naphthyl)-ureido]-pyrazol-1-yl}-phenoxy)-acetic acid is synthesized.

### Example QQ

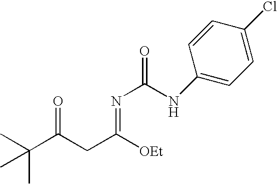

Example HH and 4-chlorophenylisocyanate are combined utilizing the same synthetic procedure as for Example II to afford (Z)-1-(4-chlorophenyl)-3-(1-ethoxy-4,4-dimethyl-3-oxopentylidene)urea

### Example 188

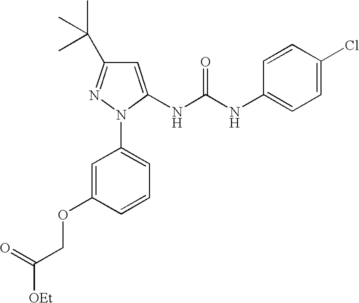

Utilizing the same synthetic procedure as for Example 164, Example QQ (10 mmol) and Example OO (10.5 mmol) are combined to afford 3-(3-{3-t-butyl-5-[3-(1-4-chlorophenyl)-ureido]-pyrazol-1-yl}-phenoxy)-acetic acid ethyl ester.

### Example 189

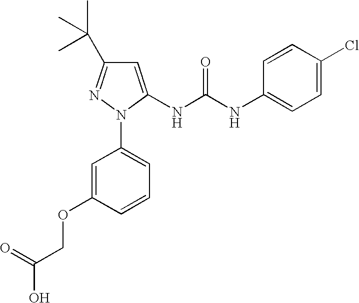

Utilizing the same synthetic procedure as for Example 146 and starting with Example 188, 3-(3-{3-t-butyl-5-[3-(14-chlorophenyl)-ureido]-pyrazol-1-yl}-phenoxy)-acetic acid is synthesized.

### Example RR

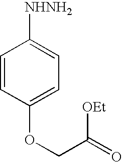

Ethyl bromoacetate is reacted with para-nitrophenol under standard conditions to afford ethyl 2-(4-nitrophenoxy)acetate, which is elaborated to ethyl 2-(4-hydrazinophenoxy)acetate using the reduction/oxidation sequence described for Example Y.

### Example 190

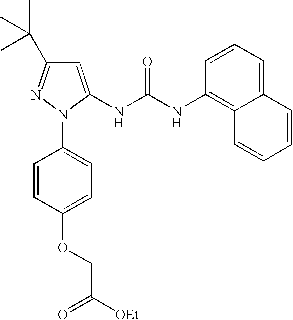

Utilizing the same synthetic procedure as for Example 164, Example PP (10 mmol) and Example RR (10.5 mmol) are combined to afford 4-(3-{3-t-butyl-5-[3-(1-naphthyl)-ureido]-pyrazol-1-yl}-phenoxy)-acetic acid ethyl ester.

### Example 191

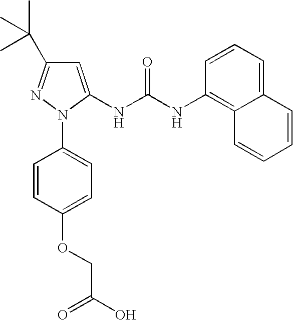

Utilizing the same synthetic procedure as for Example 146 and starting with Example 190, 4-(3-{3-t-butyl-5-[3-(1-naphthyl)-ureido]-pyrazol-1-yl}-phenoxy)-acetic acid is synthesized.

### Example 192

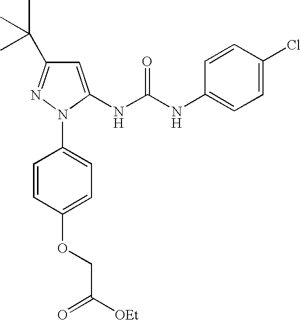

Utilizing the same synthetic procedure as for Example 164, Example QQ (10 mmol) and Example RR (10.5 mmol) are combined to afford 4-(3-{3-t-butyl-5-[3-(1-4-chlorophenyl)-ureido]-pyrazol-1-yl}-phenoxy)-acetic acid ethyl ester.

### Example 193

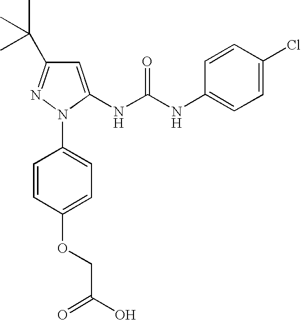

Utilizing the same synthetic procedure as for Example 146 and starting with Example 192, 4-(3-{3-t-butyl-5-[3-(14-chlorophenyl)-ureido]-pyrazol-1-yl}-phenoxy)-acetic acid is synthesized.

### Example DD

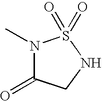

To a stirred solution of chlorosulfonyl isocyanate (1.43 g, 10.0 mmol) in CH₂Cl₂ (20 mL) at 0° C. was added 2-methyl-propan-2-ol (0.74 g, 10.0 mmol) at such a rate that the reaction solution temperature did not rise above 5° C. After being stirred for 1.5 h, a solution of glycine ethyl ester (1.45 g, 12.0 mmol) and Et₃N (3.2 mL, 25.0 mmol) in CH₂Cl₂ (20 mL) was added at such a rate that the reaction temperature didn't rise above 5° C. When the addition was completed, the solution was warmed to RT and stirred overnight. The reaction mixture was poured into 10% HCl and extracted with CH₂Cl₂. The organic layer was washed with saturated NaCl, dried (Mg₂SO₄) and filtered. After removal of the solvent, the crude product was washed with CH₂Cl₂ to afford ethyl 2-((N-(butyloxycarbonyl)sulfamoyl)amino)acetate (2.4 g, 85%). ¹H-NMR (DMSO): δ 10.85 (s, 1H), 8.04 (t, J=6.0 Hz, 1H), 4.07 (q, J=5.6 Hz, 2H), 3.77 (d, J=6.0 Hz, 2H), 1.40 (s, 9H), 1.18 (t, J=7.2 Hz, 3H).

To a solution of methanol (8.5 mmol) and triphenylphosphine (2.6 g, 8.5 mol) in dry THF is added a solution of ethyl 2-((N-(butyloxycarbonyl)sulfamoyl)amino)acetate from the previous step (2.4 g, 8.5 mol) and DIAD (2.0 g, 8.5 mmol) in dry THF dropwise at 0° C. under N₂ atmosphere. The mixture is stirred at 0° C. for 2 h, warmed to RT and is stirred overnight. After the solvent is removed in vacuo, the residue is purified by column chromatography to afford ethyl 2-((N-(butyloxycarbonyl)-N-methylsulfamoyl)amino)acetate.

To a solution of HCl in methanol (2 M) is added ethyl 2((N-(butyloxycarbonyl)-N-methylsulfamoyl)amino)acetate from the previous step (5.0 mmol) in portions at RT and the mixture is stirred for 3 h. After the solvent is removed in vacuo, the residue is washed with diethyl ether to afford ethyl 2-((N-methylsulfamoyl)amino)acetate

To a solution of ethyl 2-((N-methylsulfamoyl)amino)acetate from the previous step (3.5 mmol) in DMF (50 mL) is added KO-t-Bu (1.56 g, 13.88 mmol) in portions under N₂ at RT. The mixture is stirred overnight then quenched with HCl/methanol (2 M). After the solvent is removed in vacuo, the residue is washed with water to afford 2-methyl-1,1-dioxo-1λ⁶-[1,2,5]thiadiazolidin-3-one (480 mg, 54%). ¹H-NMR(CDCl₃): δ 7.36 (d, J=8.4 Hz, 2H), 6.87 (d, J=8.8 Hz, 2H), 4.87 (m, 1H), 4.68 (s, 2H), 4.03 (d, J=7.2 Hz, 2H), 3.80 (s, 3H).

### Example 194

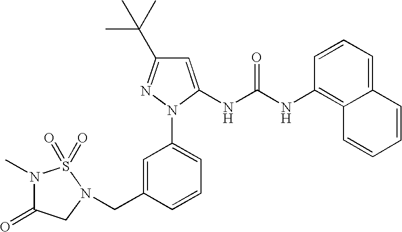

Example E and Example OO are combined utilizing the procedure for Example 160 to afford 1-(5-t-butyl-2-{3-[5-methyl-1,1,4-trioxo-1λ⁶-[1,2,5]thiadiazolidin-2-ylmethyl]-phenyl}-2H-pyrazol-3-yl)-3-naphthyl-urea.

### Example 195

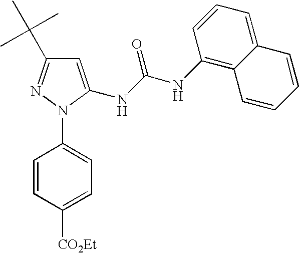

To a solution of Example X (2.9 g, 10 mmol) in THF (50 mL) was added a solution of 1-naphthyl isocyanate (1.7 g, 10 mmol) in THF (20 mL) at 0° C. The mixture was stirred at RT for 1 h and heated until all solids dissolved. The mixture was then stirred at RT for 3 h and poured into water (200 mL). The precipitate was filtered, washed with diluted HCl and H₂O, dried under vacuum to give 4.3 g of 4-[3-t-butyl-5-(3-naphthalen-1-yl-ureido)-pyrazol-1-yl]-benzoic acid ethyl ester, which was used without further purification.

### Example 196

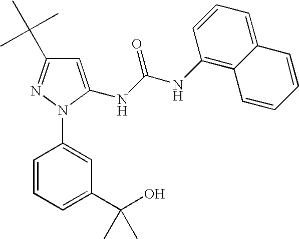

To a solution of Example B (228 mg, 0.5 mmol) in dry THF (20 mL) was added dropwise a solution of methyl magnesium bromide in toluene/THF (3.6 mL, 5.0 mmol) at −78° C. under N₂. After stirring for 1 h, the mixture was allowed to rise to RT and stirred for another 2 h. The reaction mixture was quenched with saturated NH₄Cl solution and aqueous HCl solution (10%), extracted with ethyl acetate. The combined organic extracts were washed with brine, dried (Na₂SO₄), the solvent removed in vacuo and the residue purified by column chromatography to afford 1-{5-t-butyl-2-[3-(1-hydroxy-1-methyl-ethyl)-phenyl]-2H-pyrazol-3-yl)-3-naphthalen-1-yl-urea (150 mg, 67%). ¹H NMR (DMSO-d6): 9.00 (s, 1H), 8.75 (s, 1H), 7.98 (d, J=7.6 Hz, 1H), 7.92-7.89 (m, 2H), 7.65-7.62 (m, 2H), 7.52-7.44 (m, 5H), 7.37 (d, J=6.8 Hz, 1H), 6.39 (s, 1H), 5.13 (s, 1H), 1.45 (s, 6H), 1.27 (s, 9H); MS (ESI) m/z: 443 (M+H⁺).

### Example 197

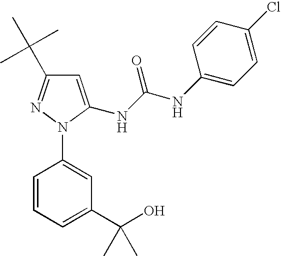

To a solution of Example C (220 mg, 0.5 mmol) in dry THF (20 mL) was added dropwise a solution of methyl magnesium bromide in toluene/THF (3.6 mL, 5.0 mmol) at −78° C. under N₂. After stirring for 1 h, the mixture was allowed to rise to RT and stirred for another 2 h. The reaction mixture was quenched with saturated NH₄Cl and aqueous HCl solution (10%), and extracted with ethyl acetate. The combined organic extracts were washed with brine, dried (Na₂SO₄), the solvent was removed in vacuo and the residue was purified by column chromatography to afford 1-{5-t-butyl-2-[3-(1-hydroxy-1-methyl-ethyl)-phenyl]-2H-pyrazol-3-yl}-3-(4-chloro-phenyl)-urea (174 mg, 81%). ¹H NMR (DMSO-d6): 9.11 (s, 1H), 8.34 (s, 1H), 7.59 (s, 1H), 7.46, (t, J=8.8 Hz, 1H), 7.43-7.40 (m, 3H), 7.31-7.28 (m, 3H), 6.34 (s, 1H), 5.13 (s, 1H), 1.42 (s, 6H), 1.27 (s, 9H); MS (ESI) m/z: 428 (M+H⁺).

### Example 198

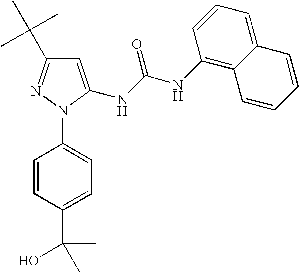

To a solution of Example 195 (228 mg, 0.5 mmol) in dry THF (20 mL) was added dropwise a solution of methylmagnesium bromide in toluene/THF (3.6 mL, 5.0 mmol) at −78° C. under N₂. After stirring for 1 h, the mixture was allowed to rise to RT and stirred for another 2 h. The reaction mixture was quenched with saturated NH₄Cl and aqueous HCl solution (10%), extracted with ethyl acetate. The combined organic extracts were washed with brine, dried (Na₂SO₄), the solvent was removed in vacuo and the residue purified by column chromatography to afford 1-{5-t-butyl-2-[4-(1-hydroxy-1-methyl-ethyl)-phenyl]-2H-pyrazol-3-yl}-3-naphthalen-1-yl-urea (180 mg, 81%). ¹H NMR (DMSO-d6): 9.06 (s, 1H), 8.83 (s, 1H), 7.99 (d, J=8.0 Hz, 1H), 7.92 (t, J=8.0 Hz, 2H), 7.64-7.61 (m, 3H), 7.55-7.43 (m, 5H), 6.40 (s, 1H), 5.13 (s, 1H), 1.47 (s, 6H), 1.27 (s, 9H); MS (ESI) m/z: 443 (M+H⁺).

### Example 199

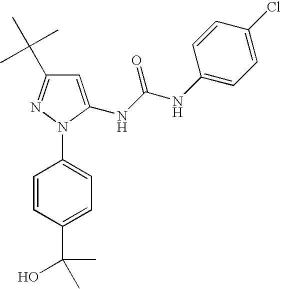

To a solution of Example 57 (220 mg, 0.5 mmol) in dry THF (20 mL) was added dropwise a solution of methyl magnesium bromide in toluene/THF (3.6 mL, 5.0 mmol) at −78° C. under N₂. After stirring for 1 h, the mixture was allowed to rise to RT and stirred for another 2 h. The reaction mixture was quenched with saturated NH₄Cl and aqueous HCl solution (10%), and extracted with ethyl acetate. The combined organic extracts were washed with brine, dried (Na₂SO₄), the solvent removed in vacuo and the residue was purified by column chromatography to afford 1-{5-t-butyl-2-[4-(1-hydroxy-1-methyl-ethyl)-phenyl]-2H-pyrazol-3-yl}-3-(4-chloro-phenyl)-urea (187 mg, 87%). ¹H-NMR (CDCl₃): 9.14 (s, 1H), 8.42 (s, 1H), 7.58 (d, J=8.4 Hz, 2H), 7.42 (d, J=5.6 Hz, 2H), 7.40 (d, J=4.8 Hz, 2H), 7.29 (d, J=8.8 Hz, H), 6.34 (s, 1H), 5.11 (s, 1H), 1.44 (s, 6H), 1.25 (s, 9H); MS (ESI) m/z: 427 (M+H⁺).

### Example PP

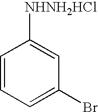

To a solution of 3-bromo-phenylamine (17 g, 0.1 mol) in concentrated HCl (200 mL) was added an aqueous solution (20 mL) of NaNO₂ (7 g, 0.1 mol) at 0° C. and the resulting mixture was stirred for 1 h. A solution of SnCl₂.2H₂O (45 g, 0.2 mmol) in concentrated HCl (500 mL) was then added at 0° C. The reaction solution was stirred for an additional 2 h at RT. The precipitate was filtered and washed with ethanol and ether to give (3-bromo-phenyl)-hydrazine as a white solid, which was used for the next reaction without further purification

### Example QQ

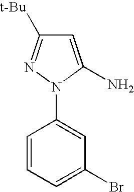

A mixture of Example PP (22.2 g, 0.1 mol) and 4,4-dimethyl-3-oxo-pentanenitrile (18.7 g, 0.15 mol) in ethanol (250 mL) was heated to reflux overnight. The reaction solution was concentrated under reduced pressure, and the residue purified by column chromatography to afford 2-(3-bromo-phenyl)-5-t-butyl-2H-pyrazol-3-ylamine as a white solid. ¹H NMR (DMSO-d₆): 7.85 (s, 1H), 7.68 (d, J=7.6 Hz, 1H), 7.62 (d, J=7.2 Hz, 1H), 7.50 (t, J=8.0 Hz, 1H), 5.62 (s, 1H), 1.27 (s, 9H).

### Example RR

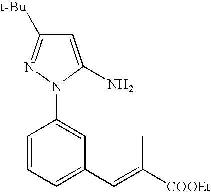

To a mixture of Example QQ (2.94 g, 10 mmol), Pd(OAc)₂ (1 mmol), PPh₃ (20 mmol), and K₂CO₃ (20 mmol) in MeCN (50 mL) was added 2-methyl-acrylic acid ethyl ester (20 mmol). The resulting mixture was heated to reflux overnight, filtered, concentrated, and the residue was purified by column chromatography to afford 1.2 g of 3-[3-(5-Amino-3-t-butyl-pyrazol-1-yl)-phenyl]-2-methyl-acrylic acid ethyl ester. ¹H NMR (CDCl₃): 7.41 (s, 1H), 7.40-7.36 (m, 2H), 7.15 (d, J=6.8 Hz, 1H), 6.24 (s, 1H), 5.51 (s, 1H), 4.27 (q, J=7.2 Hz, 2H), 2.12 (s, 3H), 1.33 (s, 9H), 1.27 (t, J=7.2 Hz, 3H).

### Example SS

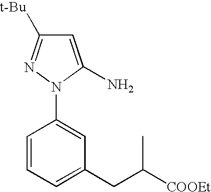

A mixture of Example RR (1.2 g,) and Pd/C (120 mg, 10%) in methanol (50 mL) was stirred under 40 psi of H₂ at RT overnight, filtered. And concentrated to afford 3-[3-(5-amino-3-t-butyl-pyrazol-1-yl)-phenyl]-2-methyl-propionic acid ethyl ester as a racemate (1.1 g), which was used for the next reaction without further purification.

### Example 200

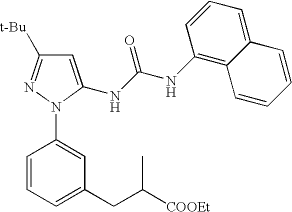

To a solution of Example SS (100 mg, 0.3 mmol) and Et₃N (60 mg, 0.6 mmol) in CH₂Cl₂ (10 mL) was added 1-isocyanato-naphthalene (77 mg, 0.45 mmol). The resulting mixture was stirred at RT overnight, added to water (50 mL), extracted with CH₂Cl₂ (3×30 mL) and the combined organic extracted were washed with brine, dried (Na₂SO₄), and filtered. After concentration under reduced pressure, the residue was purified by preparative-TLC to afford 3-(3-{3-t-butyl-5-[3-(4-fluoro-phenyl)-ureido]-pyrazol-1-yl}-phenyl)-propionic acid ethyl ester as a racemate (50 mg, 33%). ¹H-NMR (CDCl₃): 7.99 (s, 1H), 7.91 (d, J=8.4 Hz, 1H), 7.84 (t, J=7.2 Hz, 2H), 7.67 (d, J=8.4 Hz, 1H), 7.49-7.41 (m, 3H), 7.35-7.33 (m, 3H), 7.21 (s, 1H), 7.14-7.13 (m, 1H), 6.65 (s, 1H), 3.98 (q, J=6.0 Hz, 2H), 2.92-2.88 (m, 3H), 1.36 (s, 9H), 1.24 (d, J=6.0 Hz, 3H), 1.08 (t, J=7.2 Hz, 3H); MS (ESI) m/z: 499 (M+H⁺).

### Example 201

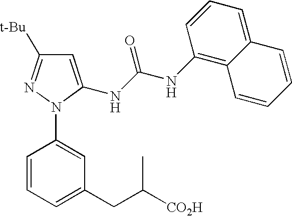

A solution of Example 200 (17 mg, mmol) and 2N LiOH (3 mL) in MeOH (3 mL) was stirred at RT over night. The reaction mixture was adjusted to pH=4, and extracted with ethyl acetate (3×20 mL). The combined organic extracts were washed with brine, dried (Na₂SO₄), and filtered. After the filtrate was concentrated, the residue was purified by preparative-TLC to afford 3-{3-[3-t-butyl-5-(3-naphthalen-1-yl-ureido)-pyrazol-1-yl]-phenyl}-2-methyl-propionic acid as a racemate (15 mg, 92%). ¹H NMR (DMSO): 11.81 (br s, 1H), 9.58 (s, 1H), 8.56 (s, 1H), 7.95 (d, J=7.6 Hz, 1H), 7.82 (d, J=8.4 Hz, 1H), 7.55 (d, J=7.6 Hz, 1H), 7.45-7.35 (m, 5H), 7.28 (d, J=8.0 Hz, 1H), 7.14 (t, J=7.6 Hz, 1H), 6.52 (s, 1H), 3.77 (m, 1H), 2.65 (m, 1H), 2.36 (m, 1H), 1.27 (s, 9H), 1.00 (d, J=6.8 Hz, 3H); MS (ESI) m/z: 471 (M+H⁺).

### Example 202

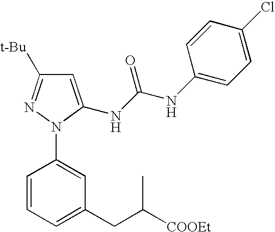

To a solution of Example SS (100 mg, 0.3 mmol) and Et₃N (60 mg, 0.6 mmol) in CH₂Cl₂ (10 mL) was added 1-chloro-4-isocyanato-benzene (77 mg, 0.45 mmol). The resulting mixture was stirred at RT overnight, and then added to water (50 mL). The solution was extracted with CH₂Cl₂ (3×30 mL) and the combined organic extracts were washed with brine, dried (Na₂SO₄), and filtered. After concentration under reduced pressure, the residue was purified by preparative-TLC to afford 3-(3-{3-t-butyl-5-[3-(4-chloro-phenyl)-ureido]-pyrazol-1-yl}-phenyl)-2-methyl-propionic acid ethyl ester as a racemate (51 mg, 35%). ¹H-NMR (CDCl₃): 8.20 (s, 1H), 7.39 (d, J=4.4 Hz, 2H), 7.37 (d, J=8.8 Hz, 2H), 7.21 (t, J=8.4 Hz, 2H), 7.14-7.11 (m, 2H), 6.59 (s, 1H), 4.04-3.99 (m, 2H), 3.00 (m, 1H), 2.93 (m, 1H), 2.83 (m, 1H), 1.34 (s, 9H), 1.17 (d, J=6.4 Hz, 3H), 1.15 (t, J=7.2 Hz, 3H); MS (ESI) m/z: 483 (M+H⁺).

### Example 203

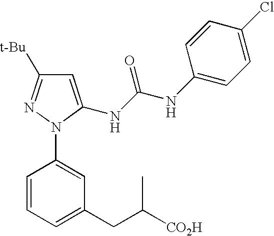

A solution of Example 202 (15 mg, mmol) and 2N LiOH (3 mL) in MeOH (3 mL) was stirred at RT overnight. The reaction mixture was adjusted to pH=4, extracted with ethyl acetate (3×20 mL), the combined organic extracts were washed with brine, dried (Na₂SO₄), and filtered. After the filtrate was concentrated, the residue was purified by preparative-TLC to afford 3-(3-{3-t-butyl-5-[3-(4-chloro-phenyl)-ureido]-pyrazol-1-yl}-phenyl)-2-methyl-propionic acid as a racemate (13 mg, 90%). ¹H NMR (DMSO): 12.48 (br s, 1H), 9.35 (br s, 1H), 7.55 (d, J=8.8 Hz, 1H), 7.34-7.32 (m, 2H), 7.26 (d, J=8.4 Hz, 1H), 7.24 (d, J=8.8 Hz, 2H), 7.10 (d, J=7.6 Hz, 1H), 6.45 (s, 1H), 2.74 (m, 1H), 2.65 (m, 1H), 2.31 (m, 2H), 1.26 (s, 9H), 0.99 (d, J=6.8 Hz, 3H); MS (ESI) m/z: 455 (M+H⁺).

### Example 204

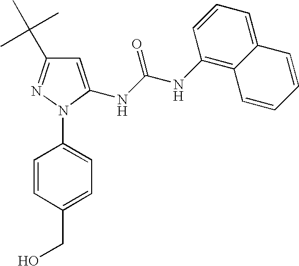

To a stirred solution of Example 195 (500 mg, 0.83 mmol) in THF (10 mL) was added LiAlH₄ powder (65 mg, 1.66 mmol) in portion at 0° C. under N₂. The mixture was stirred for 2 h at RT, excess LiAlH₄ was destroyed by a slow addition of ice, and the reaction mixture was acidified to pH=7 with dilute HCl. After the solvent was removed, the residue was extracted with ethyl acetate. The combined organic extracts were washed with brine, dried (Na₂SO₄), and filtered. After concentration in vacuo, the crude product was purified by preparative-TLC to afford 1-[2-(4-hydroxymethyl-phenyl)-5-isopropyl-2H-pyrazol-3-yl]-3-naphthalen-1-yl-urea (415 mg, 92%). ¹H NMR (DMSO-d₆): 9.04 (s, 1H), 8.78 (s, 1H), 7.98 (d, J=8.0 Hz, 1H), 7.90 (d, J=7.2 Hz, 2H), 7.63 (d, J=8.4 Hz, 1H), 7.55-7.42 (m, 7H), 6.39 (s, 1H), 5.30 (t, J=5.6 Hz, 1H), 4.56 (d, J=5.6 Hz, 2H), 1.27 (s, 9H); MS (ESI) m/z: 415 (M+H⁺).

### Example 205

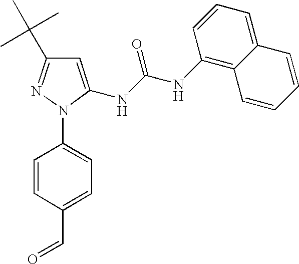

To a solution of Example 204 (200 mg) in CH₂Cl₂ (50 mL) was added MnO₂ (450 mg) at RT. The suspension was stirred for 2 h then filtered through celite. The filtrate was concentrated under reduced pressure to afford 150 mg of 1-[5-t-butyl-2-(4-formyl-phenyl)-2H-pyrazol-3-yl]-3-naphthalen-1-yl-urea, which was used without further purification.

### Example 206

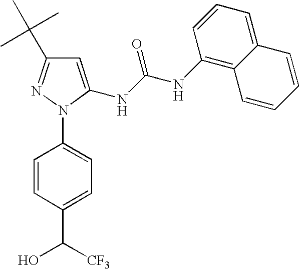

To a solution of (trifluoromethyl)trimethylsilane (77 mg) and TBAF (10 mg) in THF (10 mL) was added Example 205 (150 mg) in THF (10 mL) under N₂ atmosphere in ice-bath. The resulting mixture was stirred at 0° C. for 1 h and then warmed to RT for an additional hour. To the reaction was then added 0.5 mL of 3 N HCL, which was then stirred at RT overnight. After removal the solvent, the residue was dissolved in CH₂Cl₂ (50 mL). The organic layer was washed with saturated NaHCO₃ and brine, dried (Na₂SO₄), and filtered. After the filtrate was concentrated under reduced pressure, the residue was purified by preparative-TLC to afford the final product 1-{5-t-Butyl-2-[4-(2,2,2-trifluoro-1-hydroxy-ethyl)-phenyl]-2H-pyrazol-3-yl}-3-naphthalen-1-yl-urea (110 mg, 63%). ¹H NMR (DMSO-d₆): 9.07 (s, 1H), 8.89 (s, 1H), 8.03 (d, J=8.0 Hz, 1H), 7.90 (d, J=7.6 Hz, 2H), 7.67-7.62 (m, 5H), 7.55-7.51 (m, 2H), 7.44 (t, J=8.0 Hz, 1H), 6.95 (d, J=6.0 Hz, 1H), 6.42 (s, 1H), 5.27 (m, 1H), 1.28 (s, 9H). MS (ESI) m/z: 483 (M+H⁺).

### Example 207

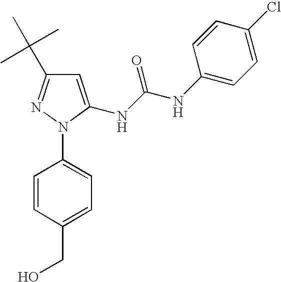

To a stirred solution of Example 57 (500 mg, 1.1 mmol) in THF (10 mL) was added LiAlH₄ powder (65 mg, 1.66 mmol) in portion at 0° C. under N₂. The mixture was stirred for 2 h at RT, excess LiAlH₄ was destroyed by a slow addition of ice, and the reaction mixture was acidified to pH=7 with diluted HCl. After the solvent removal, the residue was extracted with ethyl acetate, and the combined organic extracts were washed with brine, d dried (Na₂SO₄), and filtered, After solvent removal, the crude product was purified by preparative TLC to 1-[5-t-butyl-2-(4-hydroxymethyl-phenyl)-2H-pyrazol-3-yl]-3-(4-chloro-phenyl)-urea (380 mg, 92%) as a white powder. ¹H-NMR (CDCl₃): 8.17 (br s, 1H), 7.22 (s, 4H), 7.17 (d, J=8.0 Hz, 2H), 7.09 (d, J=8.0 Hz, 2H), 7.04 (s, H), 6.38 (s, 1H), 4.51 (s, 1H), 1.22 (s, 9H); MS (ESI) m/z: 399 (M+H⁺).

### Example 208

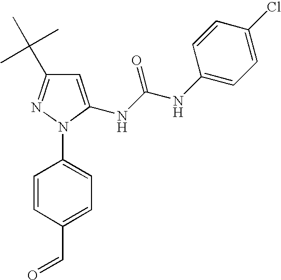

To a solution of Example 207 (200 mg) in CH₂Cl₂ (50 mL) was added MnO₂ (450 mg) at RT. The suspension was stirred for 2 h, then filtered through celite. The filtrate was concentrated to afford 160 mg of 1-[5-t-butyl-2-(4-formyl-phenyl)-2H-pyrazol-3-yl]-3-(4-chloro-phenyl)-urea, which was used without further purification.

### Example 209

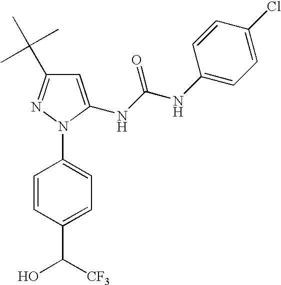

To a solution of (trifluoromethyl)trimethylsilane (86 mg) and TBAF (10 mg) in THF (10 mL) was added Example 208 (160 mg) in THF (20 mL) under N₂ atmosphere in ice-bath. The resulting mixture was stirred at 0° C. for 1 h and then warmed to RT for an additional hour. To the reaction was added 0.5 mL of 3 N HCl, which was then stirred at RT overnight. After removal of the solvent, the residue was dissolved in CH₂Cl₂ (100 mL). The organic extracts were washed with saturated NaHCO₃ and brine, dried (Na₂SO₄), and filtered. After the filtrate was concentrated under reduced pressure, the residue was purified by preparative-TLC to afford the final product 1-{5-t-butyl-2-[4-(2,2,2-trifluoro-1-hydroxy-ethyl)-phenyl]-2H-pyrazol-3-yl}-3-(4-chloro-phenyl)-urea (120 mg, 64%) ¹H-NMR (DMSO-d6): 9.15(s, 1H), 8.50 (s, 1H), 7.61 (d, J=8.4 Hz, 2H), 7.55 (d, J=8.4 Hz, 2H), 7.42 (d, J=8.8 Hz, 2H), 7.28 (d, J=8.8 Hz, 2H), 6.91 (d, J=5.6 Hz, 1H), 6.36 (s, 1H), 5.25 (m, 1H), 1.26 (s, 9H); MS (ESI) m/z: 467 (M+H⁺).

### Example 210

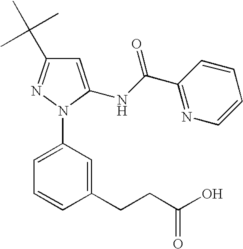

A solution of Example 151 (42 mg, 0.1 mmol) and 2N LiOH (3 mL) in MeOH (3 mL) was stirred at RT over night. The reaction mixture was neutralized to pH=4, and extracted with ethyl acetate (3×20 mL). The combined organic extracts were washed with brine, dried (Na₂SO₄), and filtered. The filtrate was concentrated to afford 3-(3-{3-t-butyl-5-[(pyridine-2-carbonyl)-amino]-pyrazol-1-yl}-phenyl)-propionic acid (30 mg, 76%). 8.45 (d, 4.0 Hz, 1H), 8.24 (d, 8.0 Hz, 1H), 7.92 (s, 1H), 7.88 (t, 7.6 Hz, 1H), 7.67 (d, 8.0 Hz, 1H), 7.36 (t, 5.6 Hz, 1H), 7.23 (t, 7.6 Hz, 1H), 6.96 (d, 6.8 Hz, 1H), 6.67 (s, 1H), 2.77 (t, 7.6 Hz, 2H), 2.22 (t, 7.6 Hz, 2H), 1.26 (s, 9H); MS (ESI) m/z: 393 (M+H⁺).

### Example 211

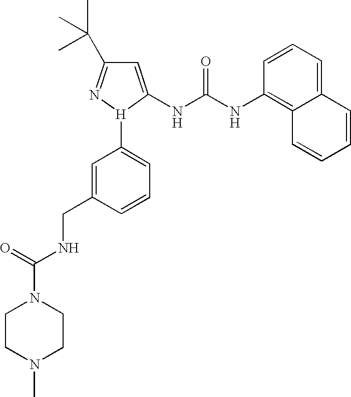

Example I is reacted with CDI and N-methyl piperazine using the procedure for Example 145 to afford the title compound.

### Example 212

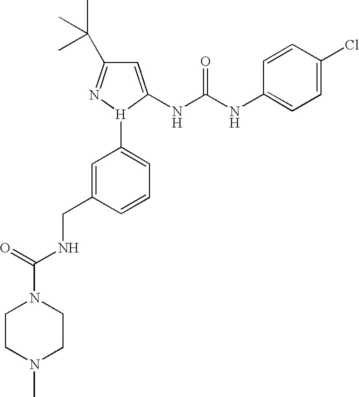

Example J is reacted with CDI and N-methyl piperazine using the procedure for Example 145 to afford the title compound.

### Example 213

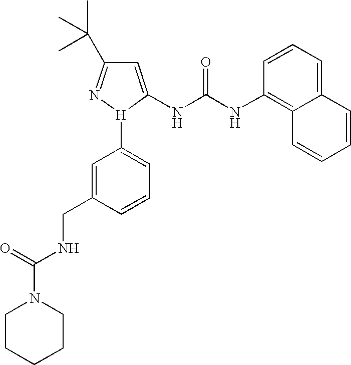

Example I is reacted with CDI and piperidine using the procedure for Example 145 to afford the title compound.

### Example 214

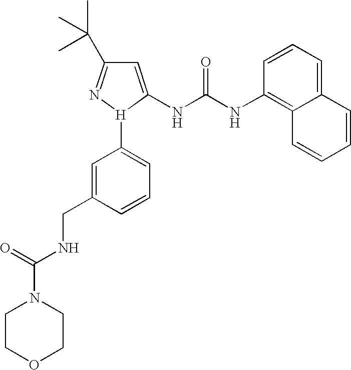

Example I is reacted with CDI and morpholine using the procedure for Example 145 to afford the title compound.

### Example 215

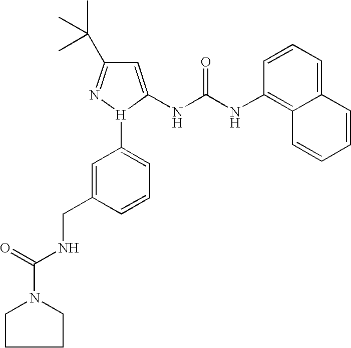

Example I is reacted with CDI and pyrrolidine using the procedure for Example 145 to afford the title compound.

### Example 216

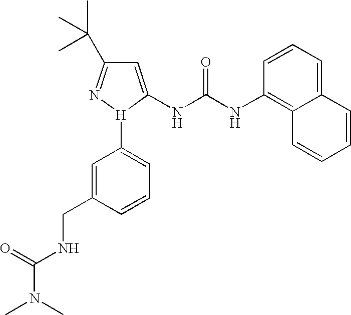

Example I is reacted with CDI and dimethylamine using the procedure for Example 145 to afford the title compound.

### Example 217

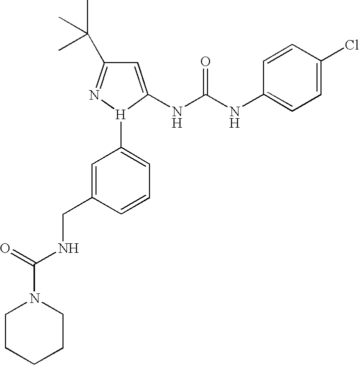

Example J is reacted with CDI and piperidine using the procedure for Example 145 to afford the title compound.

### Example 218

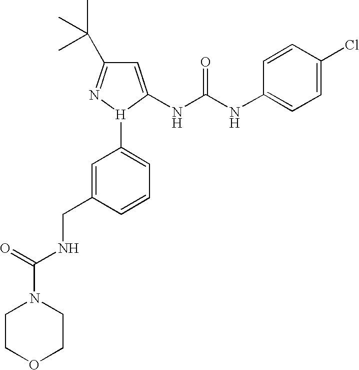

Example J is reacted with CDI and morpholine using the procedure for Example 145 to afford the title compound.

### Example 219

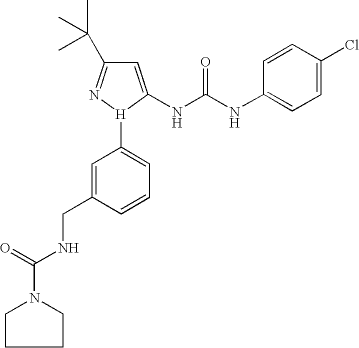

Example J is reacted with CDI and pyrrolidine using the procedure for Example 145 to afford the title compound.

### Example 220

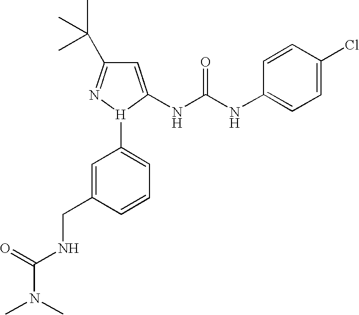

Example J is reacted with CDI and dimethylamine using the procedure for Example 145 to afford the title compound.

### Example 221

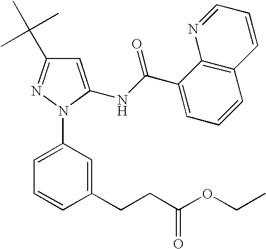

Isoquinoline-8-carboxylic acid and Example Z are reacted using the procedure for Example 149 to afford ethyl 3-(3-(3-t-butyl-5-(quinoline-8-carboxamido)-1H-pyrazol-1-yl)phenyl)propanoate.

### Example 222

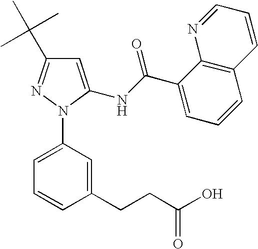

Example 222 is reacted using the procedure for Example 150 to afford 3-(3-(3-t-butyl-5-(quinoline-8-carboxamido)-1H-pyrazol-1-yl)phenyl)propanoic acid.

### Example 223

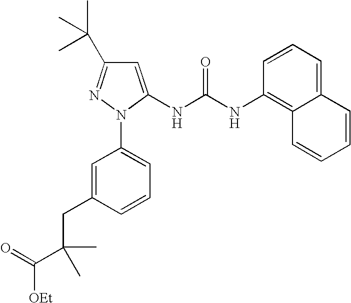

Example E is reacted with (1-ethoxy-2-methylprop-1-enyloxy)trimethylsilane under literature conditions to afford ethyl 2-(3-(3-t-butyl-5-(3-(naphthalen-1-yl)ureido)-1H-pyrazol-1-yl)benzyl)-2-methylpropanoate.

### Example 224

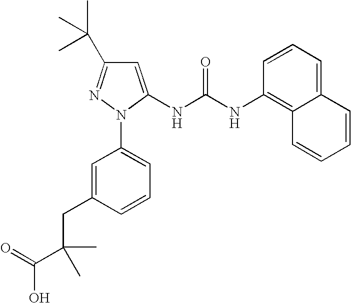

Example 224 is reacted using the procedure for Example 150 to afford 2-(3-(3-t-butyl-5-(3-(naphthalen-1-yl)ureido)-1H-pyrazol-1-yl)benzyl)-2-methylpropanoic acid

### Example 225

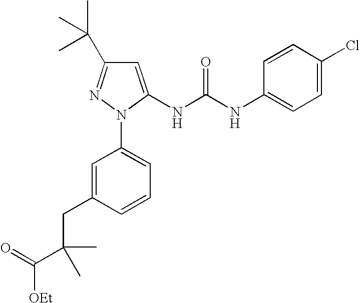

Example G is reacted with (1-ethoxy-2-methylprop-1-enyloxy)trimethylsilane under literature conditions to afford ethyl 2-(3-(3-t-butyl-5-(3-(4-chlorophenyl)ureido)-1H-pyrazol-1-yl)benzyl)-2-methylpropanoate.

### Example 226

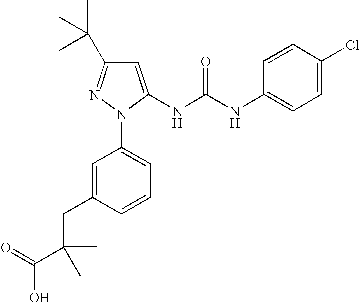

Example 226 is reacted using the procedure for Example 150 to afford 2-(3-(3-t-butyl-5-(3-(4-chlorophenyl)ureido)-1H-pyrazol-1-yl)benzyl)-2-methylpropanoic acid.

### Example TT

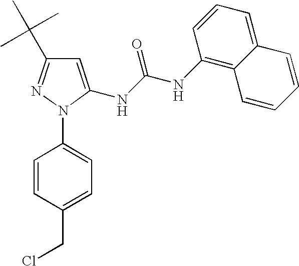

Example 204 is reacted using the procedure for Example E to afford 1-(3-t-butyl-1-(4-(chloromethyl)phenyl)-1H-pyrazol-5-yl)-3-(naphthalen-1-yl)urea

### Example UU

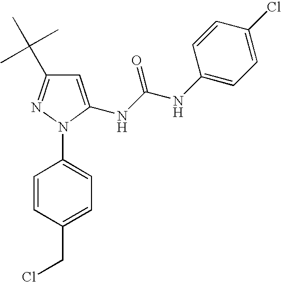

Example 122 is reacted using the procedure for Example G to afford 1-(3-t-butyl-1-(4-(chloromethyl)phenyl)-1H-pyrazol-5-yl)-3-(4-chlorophenyl)urea.

### Example 227

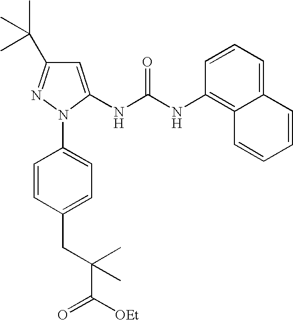

Example TT is reacted with (1-ethoxy-2-methylprop-1-enyloxy)trimethylsilane under literature conditions to afford ethyl 2-(4-(3-t-butyl-5-(3-(naphthalen-1-yl)ureido)-1H-pyrazol-1-yl)benzyl)-2-methylpropanoate.

### Example 228

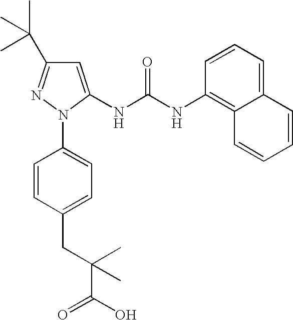

Example 227 is reacted using the procedure for Example 150 to afford 2-(4-(3-t-butyl-5-(3-(naphthalen-1-yl)ureido)-1H-pyrazol-1-yl)benzyl)-2-methylpropanoic acid.

### Example 229

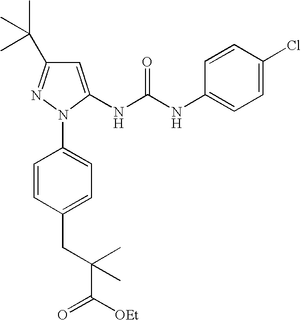

Example UU is reacted with (1-ethoxy-2-methylprop-1-enyloxy)trimethylsilane under literature conditions to afford ethyl 2-(4-(3-t-butyl-5-(3-(4-chlorophenyl)ureido)-1H-pyrazol-1-yl)benzyl)-2-methylpropanoate

### Example 230

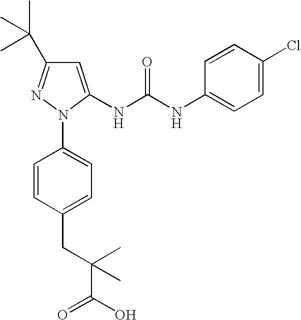

Example 144 is reacted using the procedure for Example 150 to afford 2-(4-(3-t-butyl-5-(3-(4-chlorophenyl)ureido)-1H-pyrazol-1-yl)benzyl)-2-methylpropanoic acid.

### Example VV

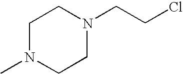

N-methyl piperazine and 1-bromo-2-chloroethane are reacted using the procedure for Example OO to afford 1-(2-chloroethyl)-4-methylpiperazine hydrochloride.

### Example WW

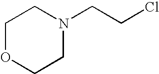

Morpholine and 1-bromo-2-chloroethane are reacted using the procedure for Example OO to afford 4-(2-chloroethyl)morpholine

### Example XX

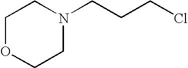

Morpholine and 1-bromo-3-chloropropane are reacted using the procedure for Example OO to afford 4-(3-chloropropyl)morpholine.

### Example YY

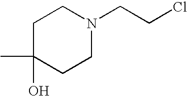

4-methylpiperidin-4-ol (made via literature methods) and 1-bromo-2-chloroethane are reacted using the procedure for Example OO to afford 1-(2-chloroethyl)-4-methylpiperidin-4-ol.

### Example ZZ

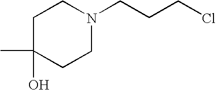

4-methylpiperidin-4-ol (made via literature methods) and 1-bromo-3-chloropropane are reacted using the procedure for Example OO to afford 1-(3-chloropropyl)-4-methylpiperidin-4-ol.

### Example AAA

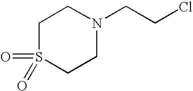

A solution of 4,4-dioxothiomorpholine and 1-bromo-2-chloroethane are reacted using the procedure for Example OO to afford 4-(2-chloroethyl)-4,4-dioxo-4-thiomorpholine.

### Example BBB

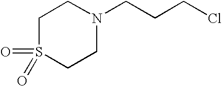

A solution of 4,4-dioxothiomorpholine and 1-bromo-3-chloropropane are reacted using the procedure for Example OO to afford 4-(3-chloropropyl)-4,4-dioxo-4-thiomorpholine.

### Example CCC

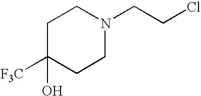

A solution of 4-(trifluoromethyl)piperidin-4-ol and 1-bromo-2-chloroethane are reacted using the procedure for Example OO to afford 1-(2-chloroethyl)-4-(trifluoromethyl)piperidin-4-ol.

### Example DDD

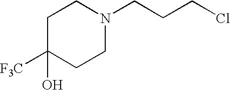

A solution of 4-(trifluoromethyl)piperidin-4-ol and 1-bromo-3-chloropropane are reacted using the procedure for Example OO to afford 1-(3-chloropropyl)-4-(trifluoromethyl)piperidin-4-ol.

### Example 231

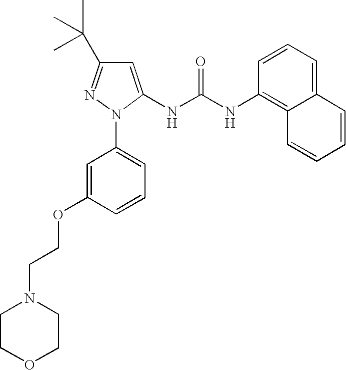

Example 41 and Example WW are reacted according to the procedure for Example 194 to afford 1-(1-(3-(2-morpholinoethoxy)phenyl)-3-t-butyl-1H-pyrazol-5-yl)-3-(naphthalen-1-yl)urea.

### Example 232

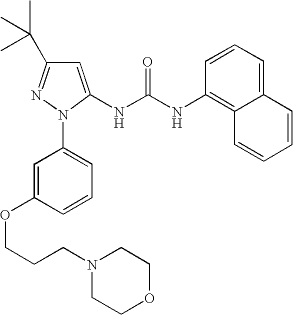

Example 41 and Example XX are reacted according to the procedure for Example 194 to afford 1-(1-(3-(3-morpholinopropoxy)phenyl)-3-t-butyl-1H-pyrazol-5-yl)-3-naphthalen-1-yl)urea.

### Example 233

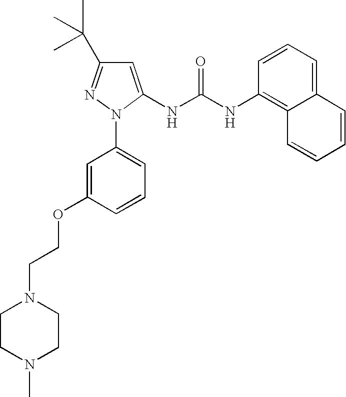

Example 41 and Example VV are reacted according to the procedure for Example 194 to afford 1-(1-(3-(2-(4-methylpiperazin-1-yl)ethoxy)phenyl)-3-t-butyl-1H-pyrazol-5-yl)-3-(naphthalen-1-yl)urea.

### Example 234

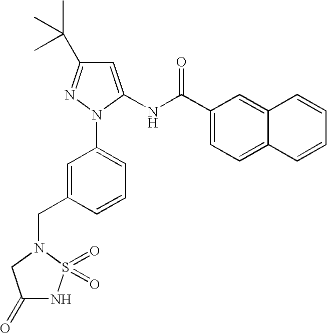

Example CC, 2-naphthoic acid chloride and Example DD were combined utilizing the same general approach for Example 162 to yield N-(3-tert-butyl-1-(3-([5-1,1,4-trioxo-1λ⁶-[1,2,5]thiadiazolidin-2-ylmethyl]phenyl)-1H-pyrazol-5-yl)-2-naphthamide. ¹H-NMR (DMSO-d₆): 10.50 (s, 1H), 8.45 (s, 1H), 8.15-8.05 (m, 3H), 7.90 (s, 1H), 7.60 (t, J=7.2 Hz, 3H), 7.45 (s, 1H), 7.38 (t, J=8.0 Hz, 1H), 7.27 (d, J=7.2 Hz, 1H), 6.44 (s, 1H), 4.05 (s, 2H), 1.31 (s, 9H). MS (ESI) m/z: 518 (M+H⁺).

### Example 235

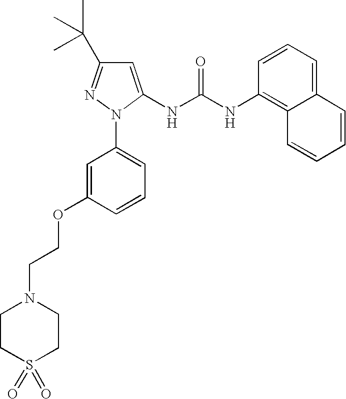

Example 41 and Example AAA are reacted according to the procedure for Example 194 to afford 1-(1-(3-(2-(4,4-dioxo-4-thio-morpholino)ethoxy)phenyl)-3-t-butyl-1H -pyrazol-5-yl)-3-(naphthalen-1-yl)urea.

### Example 236

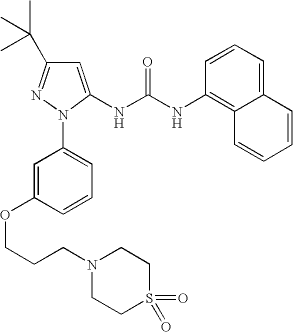

Example 41 and Example BBB are reacted according to the procedure for Example 194 to afford 1-(1-(3-(2-(4,4-dioxo-4-thio-morpholino)propoxy)phenyl)-3-t-butyl-1H-pyrazol-5-yl)-3-(naphthalen-1-yl)urea.

### Example 237

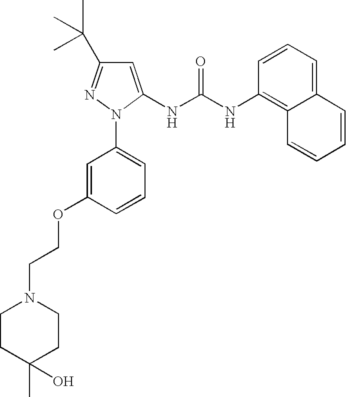

Example 41 and Example YY are reacted according to the procedure for Example 194 to afford 1-(1-(3-(2-(4-methylpiperidin-4-ol)ethoxy)phenyl)-3-t-butyl-1H-pyrazol-5-yl)-3-(naphthalen-1-yl)urea.

### Example 238

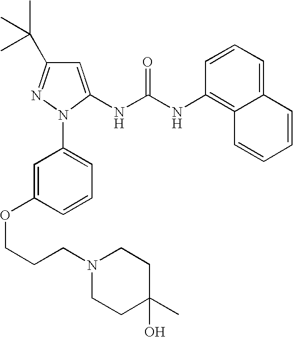

Example 41 and Example ZZ are reacted according to the procedure for Example 194 to afford 1-(1-(3-(3-(4-methylpiperidin-4-ol-)propoxy)phenyl)-3-t-butyl-1H-pyrazol-5-yl)-3-(naphthalen-1-yl)urea.

### Example 239

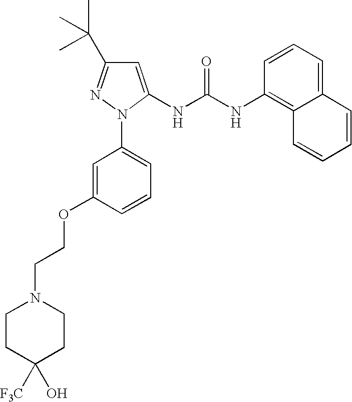

Example 41 and Example CCC are reacted according to the procedure for Example 194 to afford 1-(1-(3-(2-(4-(trifluoromethyl)piperidin-4-ol)ethoxy)phenyl)-3-t-butyl-1H-pyrazol-5-yl)-3-(naphthalen-1-yl)urea.

### Example 240

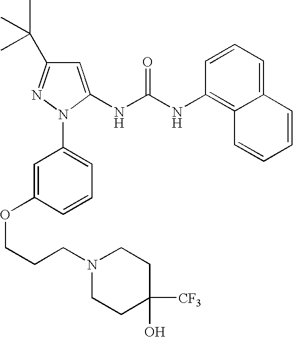

Example 41 and Example DDD are reacted according to the procedure for Example 194 to afford 1-(1-(3-(3-(4-(trifluoromethyl)piperidin-4-ol)propoxy)phenyl)-3-t-butyl-1H-pyrazol-5-yl)-3-(naphthalen-1-yl)urea.

### Example 241

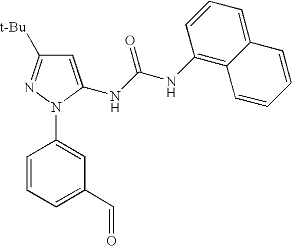

Example D is reacted using the procedure for Example 205 to afford 1-(3-t-butyl-1-(3-formylphenyl)-1H-pyrazol-5-yl)-3-(naphthalen-1-yl)urea.

### Example 242

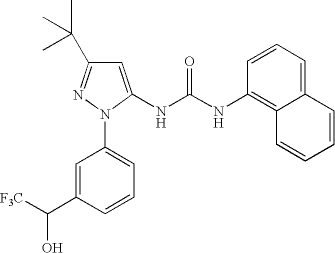

Example 242 is reacted using the procedure for Example 206 to afford 1-(3-t-butyl-1-(3-(2,2,2-trifluoro-1-hydroxyethyl)phenyl)-1H-pyrazol-5-yl)-3-(naphthalen-1-yl)urea.

### Example 243

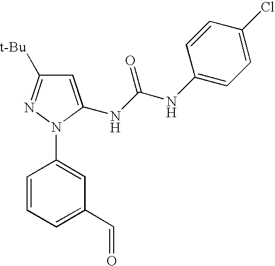

Example F is reacted using the procedure for Example 208 to afford 1-(3-t-butyl-1-(3-formylphenyl)-1H-pyrazol-5-yl)-3-(4-chlorophenyl)urea.

### Example 244

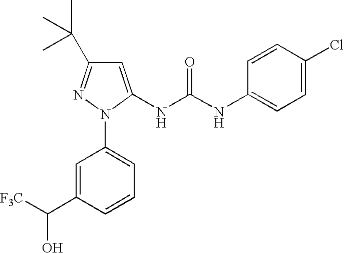

Example 244 is reacted using the procedure for Example 209 to afford 1-(3-t-butyl-1-(3-(2,2,2-trifluoro-1-hydroxyethyl)phenyl)-1H-pyrazol-5-yl)-3-(4-chlorophenyl)urea

### Example 245

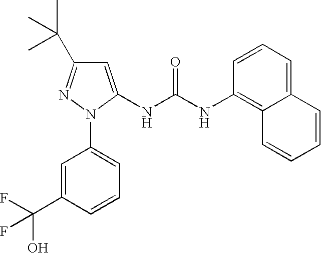

Example B is saponified using the procedure for Example 150. The resulting acid is reacted with trifluorotriazine in pyridine to afford the acid fluoride which is directly reacted with CsF and TBAF according to literature procedures (see J. Org. Chem. USSR (Engl. Transl.), 11, 1975, 315-317) to afford 1-(3-t-butyl-1-(3-(difluoro(hydroxy)methyl)phenyl)-1H-pyrazol-5-yl)-3-(naphthalen-1-yl)urea.

### Example 246

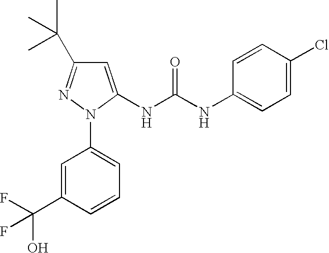

Example C is reacted according to the procedure for Example 245 to afford 1-(3-t-butyl-1-(3-(difluoro(hydroxy)methyl)phenyl)-1H-pyrazol-5-yl)-3-(4-chlorophenyl)urea.

### Example 247

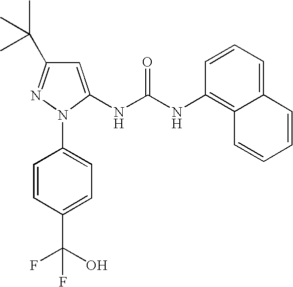

Example 205 is reacted according to the procedure for Example 245 to afford 1-(3-t-butyl-1-(4-(difluoro(hydroxy)methyl)phenyl)-1H-pyrazol-5-yl)-3-(naphthalen-1-yl)urea.

### Example 248

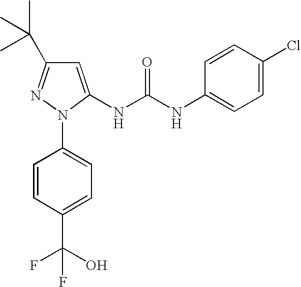

Example 57 is reacted according to the procedure for Example 245 to afford 1-(3-t-butyl-1-(4-(difluoro(hydroxy)methyl)phenyl)-1H-pyrazol-5-yl)-3-(4-chlorophenyl)urea.

### Example EEE

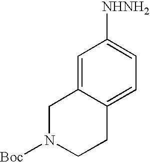

Example EEE (tert-butyl 7-hydrazinyl-3,4-dihydroisoquinoline-2(1H)-carboxylate) was synthesized according to literature procedures.

### Example 249

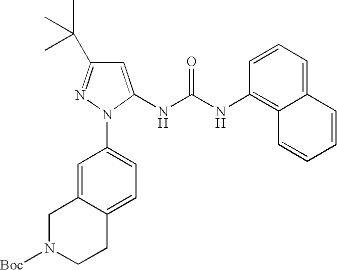

Utilizing the same synthetic procedure as for Example 164, Example EEE (10 mmol) and Example PP (10.5 mmol) are combined to afford t-butyl 7-(3-t-butyl-5-(3-(naphthalen-1-yl)ureido)-1H-pyrazol-1-yl)-3,4-dihydroisoquinoline-2(1H)-carboxylate.

### Example 250

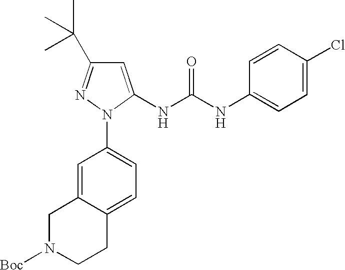

Utilizing the same synthetic procedure as for Example 164, Example EEE (10 mmol) and Example QQ (10.5 mmol) are combined to afford t-butyl 7-(3-t-butyl-5-(3-(4-chlorophenyl)ureido)-1H-pyrazol-1-yl)-3,4-dihydroisoquinoline-2(1H)-carboxylate

### Example 251

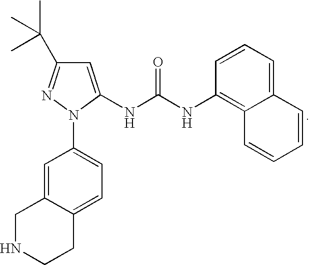

Example 249 is reacted with trifluoroacetic acid under standard conditions to afford 1-(3-t-butyl-1-(1,2,3,4-tetrahydroisoquinolin-7-yl)-1H-pyrazol-5-yl)-3-(naphthalen-1-yl)urea.

### Example 252

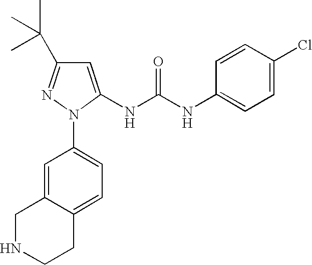

Example 250 is reacted with trifluoroacetic acid under standard conditions to afford 1-(3-t-butyl-1-(1,2,3,4-tetrahydroisoquinolin-7-yl)-1H-pyrazol-5-yl)-3-(4-chlorophenyl)urea.

### Example 253

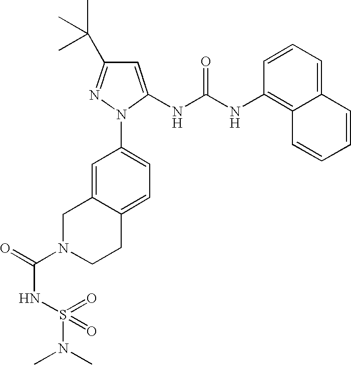

Example 251 is reacted with chlorosulfonyl isocyanate then dimethylamine according to the procedure for Example 7 to afford 1-(3-t-butyl-1-[[1-N-[[(1-dimethylaminolsulphonyl)amino]carbonyl]-1-(1,2,3,4-tetrahydroisoquinolin-7-yl)-1H-pyrazol-5-yl)-3-(naphthalen-1-yl)urea.

### Example 254

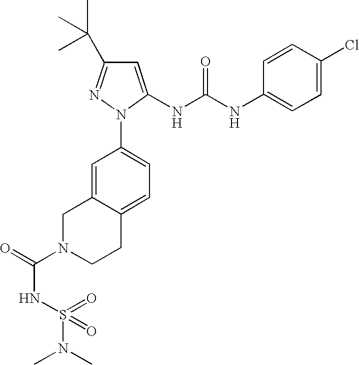

Example 252 is reacted with chlorosulfonyl isocyanate then dimethylamine according to the procedure for Example 7 to afford 1-(3-t-butyl-1-[[1-N-[[(1-dimethylaminolsulphonyl)amino]carbonyl]-1-(1,2,3,4-tetrahydroisoquinolin-7-yl)-1H-pyrazol-5-yl)-3-(4-chlorophenyl)urea.

### Example 255

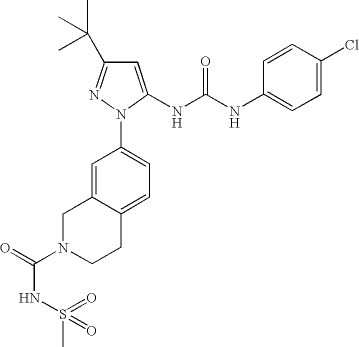

Example 252, CDI, and methanesulfonamide are reacted under standard conditions to yield 1-(3-t-butyl-1-[[1-N-[[(methanesulphonyl)amino]carbonyl]-1-(1,2,3,4-tetrahydro-isoquinolin-7-yl)-1H-pyrazol-5-yl)-3-(4-chlorophenyl)urea.

### Example 256

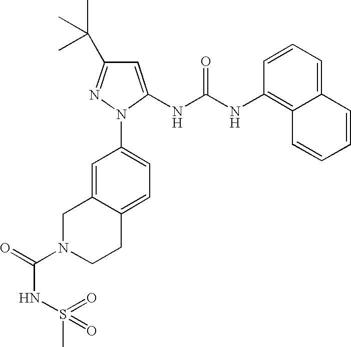

Example 251, CDI, and methanesulfonamide are reacted under standard conditions to yield 1-(3-t-butyl-1-[[1-N-[[(methanesulphonyl)amino]carbonyl]-1-(1,2,3,4-tetrahydroisoquinolin-7-yl)-1H-pyrazol-5-yl)-3-(1-naphthyl)urea.

### Example FFF

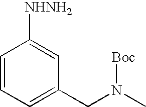

Commercially available 3-nitrobenzoic acid is reacted with methylamine and EDC under standard conditions to afford N-methyl-3-nitrobenzamide, which is reduced with LAH under standard conditions to afford N-methyl(3-nitrophenyl)methanamine, which is protected with benzylchloroformate under standard conditions to yield t-butyl 3-nitrobenzylmethylcarbamate. This material is nitrosated and reduced to yield t-butyl 3-hydrazinobenzylmethylcarbamate.

### Example 257

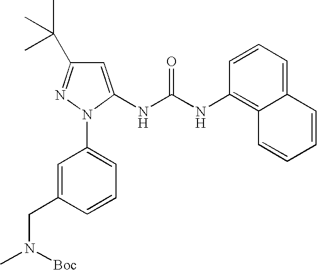

Utilizing the same synthetic procedure as for Example 164, Example FFF (10 mmol) and Example PP (10.5 mmol) are combined to afford t-butyl 3-(3-t-butyl-5-(3-(naphthalen-1-yl)ureido)-1H-pyrazol-1-yl)benzylmethylcarbamate.

### Example 258

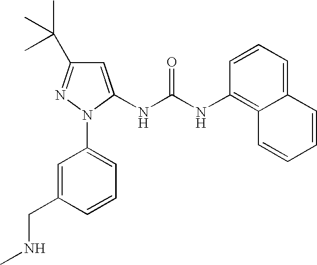

Example 257 is deprotected with trifluoroacetic acid under standard conditions to afford 1-(3-t-butyl-1-(3-((methylamino)methyl)phenyl)-1H-pyrazol-5-yl)-3-(naphthalen-1-yl)urea.

### Example 259

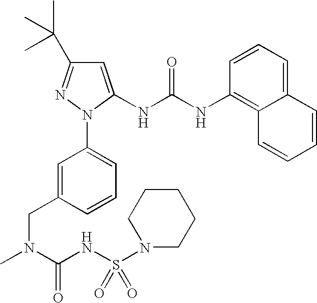

Example 258 is reacted with chlorosulfonyl isocyanate then piperidine according to the procedure for Example 7 to afford 1-(3-t-butyl-1-[[3-[[(1-piperdinylsulphonyl)amino]carbonyl]-((methylamino)methyl)phenyl)-1H-pyrazol-5-yl)-3-(naphthalen-1-yl)urea.

### Example 260

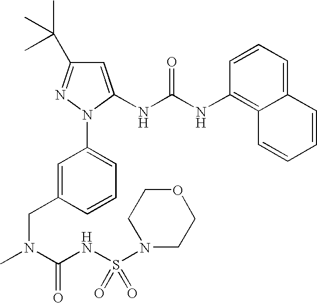

Example 258 is reacted with chlorosulfonyl isocyanate then morpholine according to the procedure for Example 7 to afford 1-(3-t-butyl-1-[[3-[[(1-morpholinyl-sulphonyl)amino]carbonyl]-((methylamino)methyl)phenyl)-1H-pyrazol-5-yl)-3-(naphthalen-1-yl)urea.

### Example 261

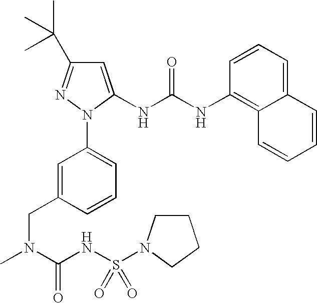

Example 258 is reacted with chlorosulfonyl isocyanate then pyrrolidine according to the procedure for Example 7 to afford 1-(3-t-butyl-1-[[3-[[(1-piperidinyl-sulphonyl)amino]carbonyl]-((methylamino)methyl)phenyl)-1H-pyrazol-5-yl)-3-(naphthalen-1-yl)urea.

### Example 262

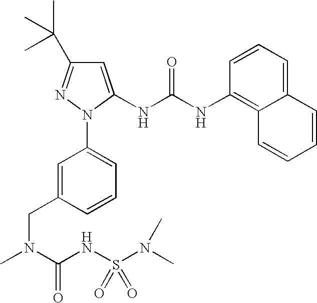

Example 258 is reacted with chlorosulfonyl isocyanate then dimethylamine according to the procedure for Example 7 to afford 1-(3-t-butyl-1-[[3-[[(1-dimethylamino-sulphonyl)amino]carbonyl]-((methylamino)methyl)phenyl)-1H-pyrazol-5-yl)-3-(naphthalen-1-yl)urea.

### Example 263

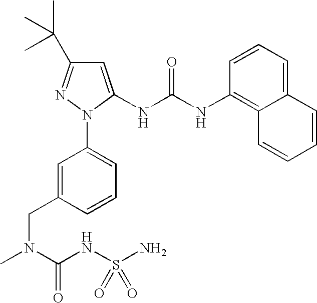

Example 258 is reacted with chlorosulfonyl isocyanate then ammonia according to the procedure for Example 7 to afford 1-(3-t-butyl-1-[[3-[[(1-aminosulphonyl)amino]carbonyl]-((methylamino)methyl)phenyl)-1H-pyrazol-5-yl)-3-(naphthalen-1-yl)urea.

### Example 264

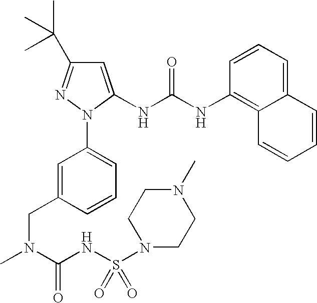

Example 258 is reacted with chlorosulfonyl isocyanate then N-methyl piperazine according to the procedure for Example 7 to afford 1-(3-t-butyl-1-[[3-[[(1-(N-methylpiperzinyl)sulphonyl)amino]carbonyl]-((methylamino)methyl)phenyl)-1H-pyrazol-5-yl)-3-(naphthalen-1-yl)urea.

### Example 265

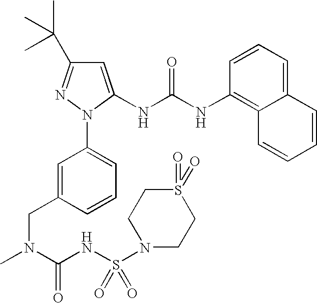

Example 258 is reacted with chlorosulfonyl isocyanate then 4,4-dioxo-4-thiomorpholine according to the procedure for Example 7 to afford 1-(3-t-butyl-1-[[3-[[(1-(4,4-dioxo-4-thiomorpholinyl)sulphonyl)amino]carbonyl]-((methylamino)methyl)phenyl)-1H-pyrazol-5-yl)-3-(naphthalen-1-yl)urea.

### Example 266

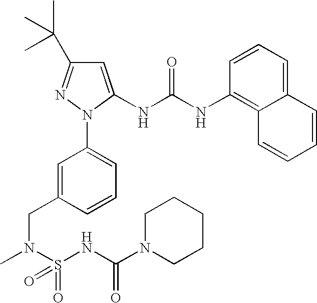

Cchlorosulfonyl isocyanate, piperidine, then Example 258 are reacted according to the procedure for Example 7 to afford 1-(3-t-butyl-1-[[3-[[(1-piperdinylcarbonyl)amino]sulphonyll]-((methylamino)methyl)phenyl)-1H-pyrazol-5-yl)-3-(naphthalen-1-yl)urea.

### Example 267

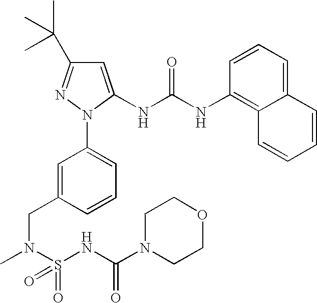

Chlorosulfonyl isocyanate, morpholine and then Example 258 are reacted according to the procedure for Example 7 to afford 1-(3-t-butyl-1-[[3-[[(1-morpholinyl-carbonyl)amino]sulphonyl]-((methylamino)methyl)phenyl)-1H-pyrazol-5-yl)-3-(naphthalen-1-yl)urea.

### Example 268

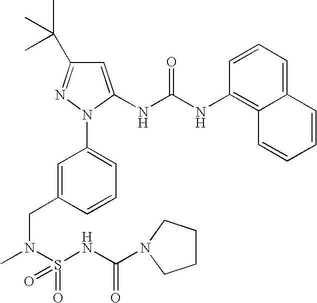

Chlorosulfonyl isocyanate, pyrrolidine and then Example 258 are reacted according to the procedure Example 7 to afford 1-(3-t-butyl-1-[[3-[[(1-piperdinylcarbonyl)amino]sulphonyl]-((methylamino)methyl)phenyl)-1H-pyrazol-5-yl)-3-(naphthalen-1-yl)urea.

### Example 269

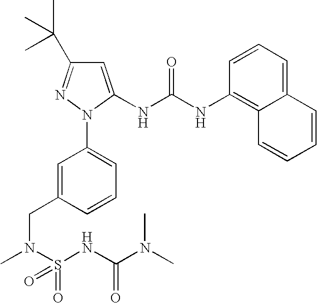

Chlorosulfonyl isocyanate, dimethylamine, then Example 258 are reacted according to the procedure for Example 7 to afford 1-(3-t-butyl-1-[[3-[[(1-dimethylamino-carbonyl)amino]sulphonyl]-((methylamino)methyl)phenyl)-1H-pyrazol-5-yl)-3-(naphthalen-1-yl)urea.

### Example 270

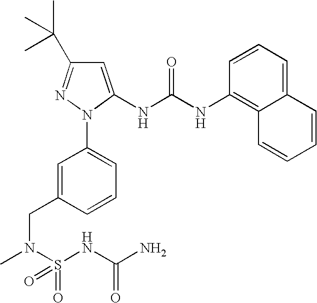

Chlorosulfonyl isocyanate, ammonia and then Example 258 are reacted according to the procedure for Example 7 to afford 1-(3-t-butyl-1-[[3-[[(1-aminocarbonyl)amino]sulphony]-((methylamino)methyl)phenyl)-1H-pyrazol-5-yl)-3-(naphthalen-1-yl)urea.

### Example 271

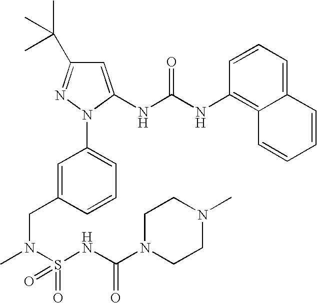

Chlorosulfonyl isocyanate, N-methyl piperazine and then Example 258 are reacted according to the procedure for Example 7 to afford 1-(3-t-butyl-1-[[3-[[(1-(N-methylpiperazinyl)carbonyl)amino]sulphonyl]-((methylamino)methyl)phenyl)-1H-pyrazol-5-yl)-3-(naphthalen-1-yl)urea.

### Example 272

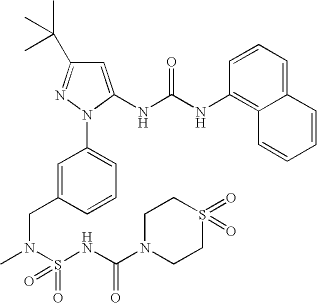

Chlorosulfonyl isocyanate, 4,4-dioxo-4-thiomorpholine and then Example 258 are reacted according to the procedure for Example 7 to afford 1-(3-t-butyl-1-[[3-[[(1-(4,4-dioxo-4-thiomorpholinyl)carbonyl)amino]sulphonyl]-((methylamino)methyl)phenyl)-1H-pyrazol-5-yl)-3-(naphthalen-1-yl)urea.

### Example GGG

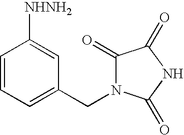

Commercially available 3-nitrobenzamide is reduced with LAH under standard conditions to afford (3-nitrophenyl)methanamine, which is reacted with 4-methoxybenzylisocyanate to afford 1-(3-nitrobenzyl)-3-(4-methoxybenzyl)urea. This material is subsequently reacted with oxalyl chloride to afford 1-(3-nitrobenzyl)-3-(4-methoxybenzyl)imidazolidine-2,4,5-trione whose nitro group is reduced and oxidized to afford 1-(3-hydrazinylbenzyl)-3-(4-methoxybenzyl)imidazolidine-2,4,5-trione. This material is deprotected with TFA under standard conditions to afford the title compound 1-(3-hydrazinylbenzyl)imidazolidine-2,4,5-trione.

### Example 273

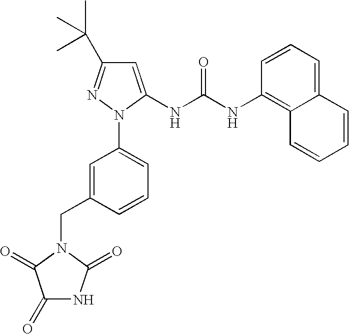

Utilizing the same synthetic procedure as for Example 164, Example GGG (10 mmol) and Example PP (10.5 mmol) are combined to afford 1-(3-t-butyl-1-(3-((2,4,5-trioxoimidazolidin-1-yl)methyl)phenyl)-1H-pyrazol-5-yl)-3-(naphthalen-1-yl)urea

### Example 274

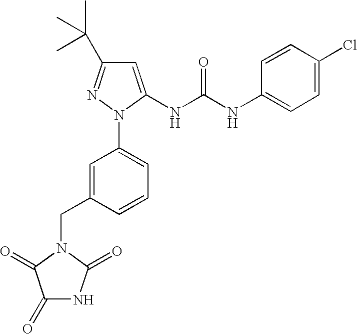

Utilizing the same synthetic procedure as for Example 164, Example GGG (10 mmol) and Example QQ (10.5 mmol) are combined to afford 1-(3-t-butyl-1-(3-((2,4,5-trioxoimidazolidin-1-yl)methyl)phenyl)-1H-pyrazol-5-yl)-3-(4-chlorophenyl)urea.

### Example HHH

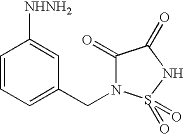

Commercially available 3-nitrobenzamide is reduced with LAH under standard conditions to afford (3-nitrophenyl)methanamine, which is reacted with N-4-methoxybenzylsulfamic acid and EDC to afford 1-(4-methoxybenzyl)-3-benzylsulfonylurea. This material is reacted with oxalyl chloride to afford 1-(3-nitrobenzyl)-3-(4-methoxybenzyl)imidazolidine-2,2-dioxo-2-thio-4,5-trione whose nitro group is reduced and oxidized to afford 1-(3-hydrazinylbenzyl)-3-(4-methoxybenzyl)imidazolidine-2,2-dioxo-2-thio-4,5-trione. This material is deprotected with TFA under standard conditions to afford the title compound 1-(3-hydrazinylbenzyl)imidazolidine-2,2-dioxo-2-thio-4,5-trione.

### Example 275

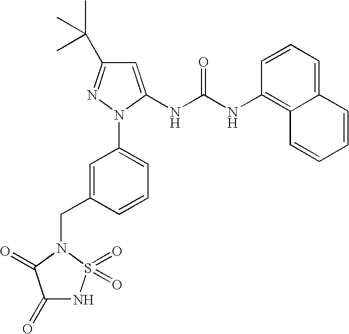

Utilizing the same synthetic procedure as for Example 164, Example HHH (10 mmol) and Example PP (10.5 mmol) are combined to afford 1-(3-t-butyl-1-(3-(2,2-dioxo-2-thio-4,5-diioxoimidazolidin-1-yl)methyl)phenyl)-1H-pyrazol-5-yl)-3-(naphthalen-1-yl)urea.

### Example 276

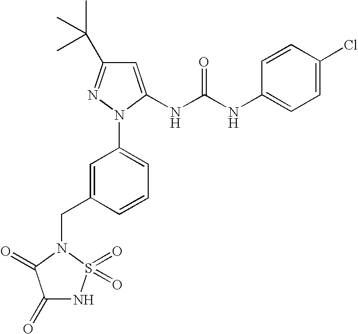

Utilizing the same synthetic procedure as for Example 164, Example HHH (10 mmol) and Example QQ (10.5 mmol) are combined to afford 1-(3-t-butyl-1-(3-((2,2-dioxo-2-thio-4,5-diioxoimidazolidin-1-yl)methyl)phenyl)-1H-pyrazol-5-yl)-3-(4-chlorophenyl)urea.

### Example 277

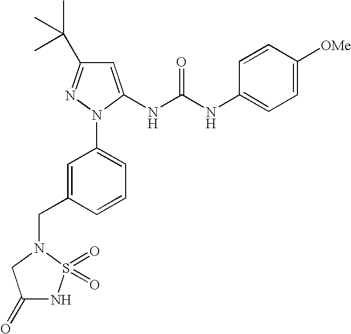

Example CC, 4-methoxy-1-aminonaphthalene and Example E are reacted using the procedure for Example 162 to afford 1-(5-t-butyl-2-{3-[1,1,4-trioxo-1λ⁶-[1,2,5]thiadiazolidin-2-ylmethyl]-phenyl}-2H-pyrazol-3-yl)-3-(4-methoxynaphth-1-yl)-urea.

### Example III

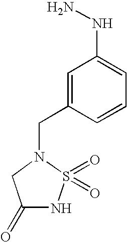

A solution of 1-(chloromethyl)-3-nitrobenzene and Example DD are combined using the procedure for Example 77 to yield 2-(4-methoxybenzyl)-5-(3-nitrophenylmethyl)-1,1-dioxo-1λ⁶-[1,2,5]thiadiazolidin-3-one. This material is reduced under standard condition to yield 2-(4-methoxybenzyl)-5-(3-aminophenylmethyl)-1,1-dioxo-1λ⁶-[1,2,5]thiadiazolidin-3-one, which was nitrosated and acidified to yield 5-(3-hydrazinophenylmethyl)-1,1-dioxo-1λ⁶-[1,2,5]thiadiazolidin-3-one.

### Example JJJ

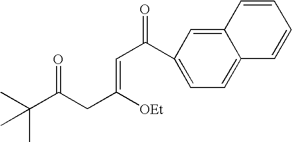

Intermediate HH (5 g, 0.0241 mol) is added to pyridine (5 mL) in CH₂Cl₂ (25 mL) and cooled in an ice bath. The suspension is stirred for 5 min and 2-naphthoic acid chloride is added dropwise over 5 min. The reaction mixture is stirred an additional 5 min at 0° C., and the reaction is warmed and stirred at RT for 1 h. The reaction is pour into ethyl acetate (100 mL) and water (100 ml). After shaking, the aqueous layer is removed, the organic layer washed with water, dried (MgSO₄) and concentrated to afford (Z)-N-(1-ethoxy-4,4-dimethyl-3-oxopentylidene)-2-naphthamide.

### Example KKK

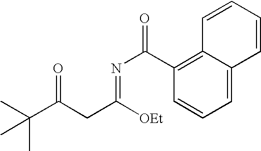

Intermediate HH (5 g, 0.0241 mol) is added to pyridine (5 mL) in CH₂Cl₂ (25 mL) and cooled in an ice bath. The suspension is stirred for 5 min and 1-naphthoic acid chloride is added dropwise over 5 min. The reaction mixture is stirred an additional 5 min at 0° C., and the reaction is warmed and stirred at RT for 1 h. The reaction is pour into ethyl acetate (100 mL) and water (100 ml). After shaking, the aqueous layer is removed, the organic layer washed with water, dried (MgSO₄) and concentrated to afford (Z)-N-(1-ethoxy-4,4-dimethyl-3-oxopentylidene)-1-naphthamide

### Example LLL

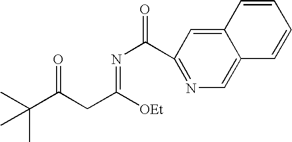

Intermediate HH (5 g, 0.0241 mol) is added to pyridine (5 mL) in CH₂Cl₂ (25 mL) and cooled in an ice bath. The suspension is stirred for 5 min and isoquinoloic acid chloride is added dropwise over 5 min. The reaction mixture is stirred an additional 5 min at 0° C., and the reaction is warmed and stirred at RT for 1 h. The reaction is pour into ethyl acetate (100 mL) and water (100 ml). After shaking, the aqueous layer is removed, the organic layer washed with water, dried (MgSO₄) and concentrated to afford (Z)-N-(1-ethoxy-4,4-dimethyl-3-oxopentylidene)isoquinoline-3-carboxamide.

### Example 278

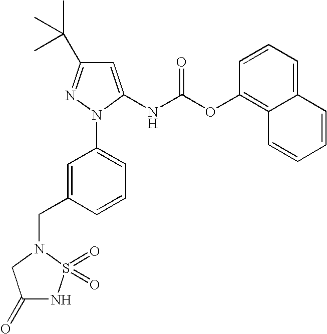

Utilizing the same synthetic procedure as for Example 164, Example III (10 mmol) and Example II (10.5 mmol) are combined to afford 1-naphtyl 1-(3-t-butyl-1-(3-((2,4,5-trioxoimidazolidin-1-yl)methyl)phenyl)-1H-pyrazol-5-yl)-3-(naphthalen-1-yl)carbamate.

### Example 279

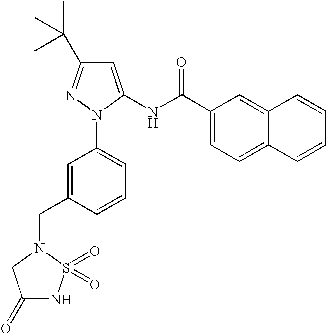

Utilizing the same synthetic procedure as for Example 164, Example III (10 mmol) and Example JJJ (10.5 mmol) are combined to afford 1-(3-t-butyl-1-(3-((2,4,5-trioxoimidazolidin-1-yl)methyl)phenyl)-1H-pyrazol-5-yl)-2-naphthamide.

### Example 280

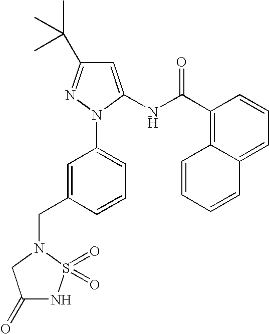

Utilizing the same synthetic procedure as for Example 164 Example III (10 mmol) and Example KKK (10.5 mmol) are combined to afford 1-(3-t-butyl-1-(3-((2,4,5-trioxoimidazolidin-1-yl)methyl)phenyl)-1H-pyrazol-5-yl)-1-naphthamide.

### Example 281

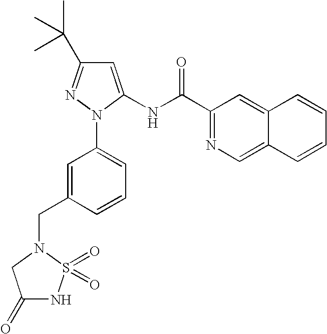

Utilizing the same synthetic procedure as for Example 149, Example III (10 mmol) and Example KKK (10.5 mmol) are combined to afford 1-(3-t-butyl-1-(3-((2,4,5-trioxoimidazolidin-1-yl)methyl)phenyl)-1H-pyrazol-5-yl)isoquinoline-3-carboxamide.

### Example MMM

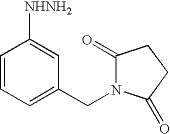

Commercially available 3-nitrobenzamide is reduced with LAH under standard conditions to afford (3-nitrophenyl)methanamine, which is reacted with succinic anhydride under standard conditions to afford 1-(3-nitrobenzyl)pyrrolidine-2,5-dione. This material is reduced at the nitro group and oxidized to afford 1-(3-hydrazinobenzyl)pyrrolidine-2,5-dione.

### Example 282

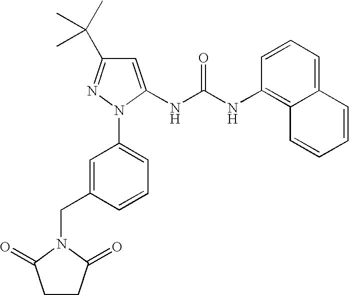

Utilizing the same synthetic procedure as for Example 164, Example MMM (10 mmol) and Example PP (10.5 mmol) are combined to afford 1-(3-t-butyl-1-(3-((2,5-dioxopyrrolidin-1-yl)methyl)phenyl)-1H-pyrazol-5-yl)-3-(naphthalen-1-yl)urea.

### Example 283

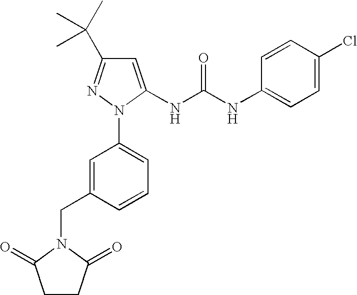

Utilizing the same synthetic procedure as for Example 164, Example MMM (10 mmol) and Example QQ (10.5 mmol) are combined to afford 1-(3-t-butyl-1-(3-((2,5-dioxopyrrolidin-1-yl)methyl)phenyl)-1H-pyrazol-5-yl)-3-(4-chlorophenyl)urea.

### Example 284

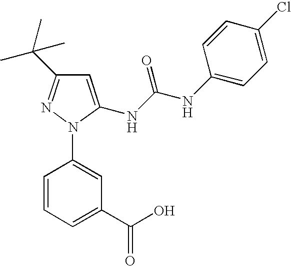

Example C was reacted with LiOH utilizing the procedure for Example 146 to yield 3-(3-t-butyl-5-(3-(4-chlorophenyl)ureido)-1H-pyrazol-1-yl)benzoic acid in 90% overall yield. ¹H NMR (DMSO-d₆): 9.00 (s, 1H), 8.83 (s, 1H), 8.25-7.42 (m, 11H), 6.42 (s, 1H), 1.26 (s, 9H); MS(ESI): Expected: 412.88 Found: 413.00.

### Example 285

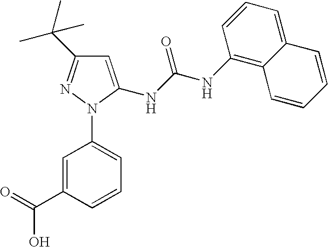

Example B was reacted with LiOH utilizing the procedure for Example 146 to yield 3-(3-t-butyl-5-(3-(naphthalen-1-yl)ureido)-1H-pyrazol-1-yl)benzoic acid in 90% overall yield. ¹H NMR (DMSO-d₆): δ 9.11 (s, 1H), 8.47 (s, 1H), 8.06 (m, 1H), 7.93 (d, J=7.6 Hz, 1H), 7.81 (d, J=8.0 Hz, 1H), 7.65 (dd, J=8.0, 7.6 Hz, 1H), 7.43 (d, J=8.8 Hz, 2H), 7.30 (d, J=8.8 Hz, 2H), 6.34 (s, 1H), 1.27 (s, 9H); MS (ESI) Expected: 428.49 Found: 429.2 (M+1).

### Example NNN

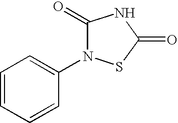

To the solution of phenyl-urea (13.0 g, 95.48 mol) in THF (100 mL) was slowly added chlorocarbonyl sulfenylchloride (13 mL, 148.85 mmol) at RT. The reaction mixture was refluxed overnight, the volatiles removed in vacuo yielded 2-phenyl-1,2,4-thiadiazolidine-3,5-dione as a white solid (4.0 g, 20%). ¹H NMR (DMSO-d₆): δ 12.49 (s, 1H), 7.51 (d, J=8.0 Hz, 2H), 7.43(t, J=7.6 Hz, 2H), 7.27 (t, J=7.2 Hz, 1H).

### Example 286

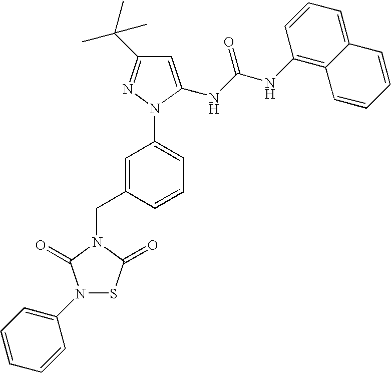

Example E and Example NNN were reacted together utilizing the same general approach as for Example 160 to afford 1-(3-t-butyl-1-(3-((3,5-dioxo-2-phenyl-1,2,4-thiadiazolidin-4-yl)methyl)phenyl)-1H-pyrazol-5-yl)-3-(naphthalen-1-yl)urea. ¹H NMR (DMSO-d₆): δ8.96 (s, 1H), 8.01-7.21 (m, 16H), 6.40 (s, 1H), 4.85 (s, 2H), 1.28 (s, 9H); MS (ESI): Expected: 590.21, Found 591.26 (M+1).

### Example 287

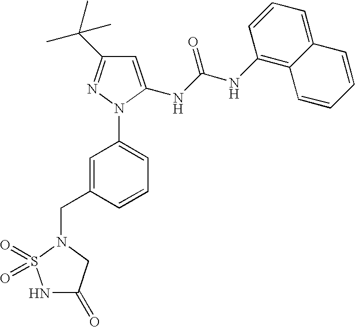

Example CC, 1-naphthylisocyanate and Example DD were combined utilizing the same general approach for Example 162 to yield 1-(5-t-butyl-2-{3-[5-1,1,4-trioxo-1λ⁶-[1,2,5]thiadia-zolidin-2-ylmethyl]-phenyl}-2H-pyrazol-3-yl)-1-naphthylurea. ¹H NMR (DMSO-d₆): δ 9.0 (s, 1H), 8.81 (s, 1H), 7.99-7.42 (m, 11H), 6.41 (s, 1H), 4.33 (s, 2H), 1.27 (s, 9H); MS (ESI) Exact Mass: 532.19 Found:=533.24.

### Example 288

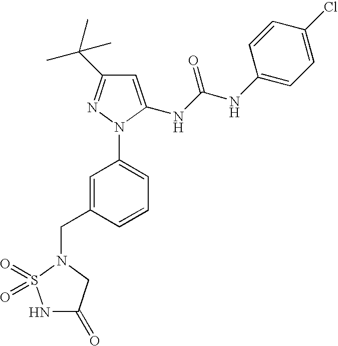

Example CC, p-chlorophenylisocyanate and Example DD were combined utilizing the same general approach for Example 162 to yield 1-(5-t-butyl-2-{3-[5-1,1,4-trioxo-1λ⁶-[1,2,5]thiadiazolidin-2-ylmethyl]-phenyl}-2H-pyrazol-3-yl)-3-(4-chloro-phenyl)-urea. ¹H NMR (DMSO-d₆): δ 9.07 (s, 1H), 8.42 (s, 1H), 7.52-7.272 (m, 8H), 6.36 (s, 1H), 4.60 (s, 2H), 1.26 (s, 9H); MS (ESI) Exact Mass: 516.13 Found:=517.1.

### Example 289

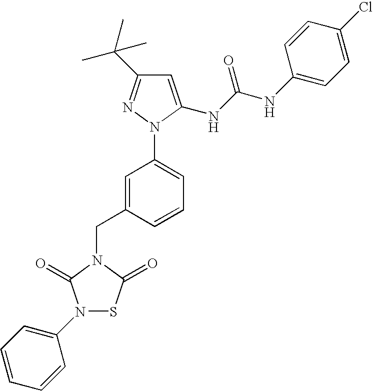

Example G and Example NNN were reacted together utilizing the same general approach as for Example 160 to afford 1-(3-t-butyl-1-(3-((3,5-dioxo-2-phenyl-1,2,4-thiadiazolidin-4-yl)methyl)phenyl)-1H-pyrazol-5-yl)-3-(4-chlorophenyl)urea. ¹H NMR (DMSO-d₆): δ9.02 (s, 1H), 8.51 (s, 1H), 7.52-7.24 (m, 13H), 6.36 (s, 1H), 4.90 (s, 2H), 1.27 (s, 9H); MS (ESI): Expected: 574.16 Found: 575.26 (M+1).

### Example 290

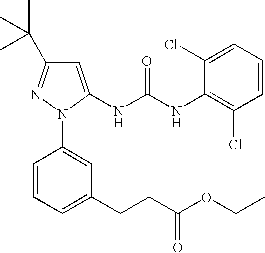

Example Z and 2,6-dichlorophenylisocyanate were reacted utilizing the same conditions as for Example 145 to yield ethyl 3-(3-(3-t-butyl-5-(3-(2,6-dichlorophenyl)ureido)-1H-pyrazol-1-yl)phenyl)propanoate. ¹H NMR (DMSO-d₆): δ 7.46-7.26 (m, 7H), 6.35 (s, 1H), 4.11 (q, J=7.2 Hz, 2H), 3.31 (t, J=5.2 Hz, 2H), 2.68 (t, J=5.6 Hz, 2H), 1.32 (s, 9H), 1.24 (t, J=7.2 Hz, 3H); MS(ESI): Expected: 502.15 Found:=503.1 (M+1).

### Example 291

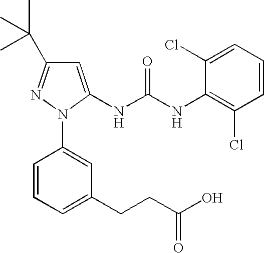

Example 290 was reacted utilizing the same condition as for Example 146 to yield 3-(3-(3-t-butyl-5-(3-(2,6-dichlorophenyl)ureido)-1H-pyrazol-1-yl)phenyl)propanoic acid in >90% yield. ¹H NMR (DMSO-d₆): δ 8.70 (s, 1H), 8.60 (s, 1H) 7.50-7.24 (m, 7H), 6.26 (s, 1H), 2.87 (t, J=5.2 Hz, 2H), 2.57 (t, J=5.6 Hz, 2H), 1.25 (s, 9H); MS(ESI): Expected: 474.12 Found: 475.18 (M+1).

### Example OOO

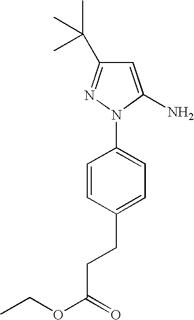

A mixture of ethyl 3-(4-aminophenyl)acrylate (1.5 g) and 10% Pd on activated carbon (0.3 g) in ethanol (20 ml) was hydrogenated at 30 psi for 18 h and filtered over Celite. Removal of the volatiles in vacuo provided ethyl 3-(4-aminophenyl)propionate (1.5 g).

A solution of the crude material from the previous reaction (1.5 g, 8.4 mmol) was dissolved in 6 N HCl (9 ml), cooled to 0° C., and vigorously stirred. Sodium nitrite (0.58 g) in water (7 ml) was added. After 1 h, tin (II) chloride dihydrate (5 g) in 6 N HCl (10 ml) was added. The reaction mixture was stirred at 0° C. for 3 h. The pH was adjusted to pH 7 to yield ethyl 3-(4-(3-t-butyl-5-amino-1H-pyrazol-1-yl)phenyl)propanoate.

### Example 292

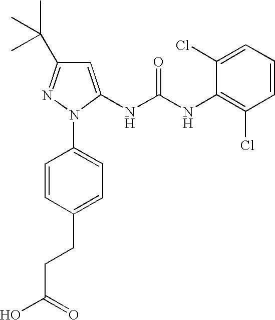

Example OOO and 2,6-dichlorophenylisocyanate were reacted utilizing the same conditions as for Example 145 to yield ethyl 3-(4-(3-t-butyl-5-(3-(2,6-dichlorophenyl)ureido)-1H-pyrazol-1-yl)phenyl)propanoate. ¹H NMR (DMSO-d₆): δ 7.45-7.24 (m, 7H), 6.36 (s, 1H), 4.10 (q, J=7.2 Hz, 2H), 3.02 (t, J=5.2 Hz, 2H), 2.70 (t, J=5.6 Hz, 2H), 1.33 (s, 9H), 1.22 (t, J=7.2 Hz, 3H); MS(ESI): Expected: 502.15 Found:=503.1 (M+1).

### Example 293

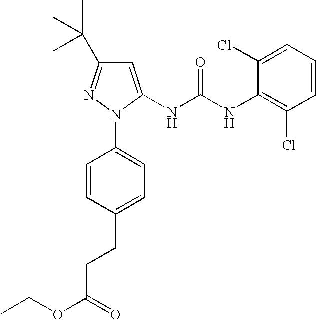

Example 292 was reacted utilizing the same condition as for Example 146 to yield 3-(3-(3-t-butyl-5-(3-(2,6-dichlorophenyl)ureido)-1H-pyrazol-1-yl)phenyl)propanoic acid in >90% yield. ¹H NMR (DMSO-d₆): δ 8.66 (s, 1H), 8.58 (s, 1H) 7.50-7.28 (m, 7H), 6.27 (s, 1H), 2.85 (t, J=5.2 Hz, 2H), 2.48 (t, J=5.6 Hz, 2H), 1.24 (s, 9H); MS(ESI): Expected: 474.12 Found: 475.18 (M+1).

### Example 294

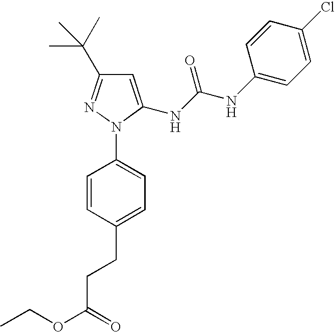

Example OOO and p-chlorophenylisocyanate were reacted utilizing the same conditions as for Example 145 to yield ethyl 3-(4-(3-tert-butyl-5-(3-(4-chlorophenyl)ureido)-1H-pyrazol-1-yl)phenyl)propanoate. ¹H NMR (DMSO-d₆): δ 7.34-7.19 (m, 9H), 6.36 (s, 1H), 4.10 (q, J=7.2 Hz, 2H), 2.92 (t, J=5.2 Hz, 2H), 2.58 (t, J=5.6 Hz, 2H), 1.32 (s, 9H), 1.25 (t, J=7.2 Hz, 3H); MS(ESI): Exact Mass: 468.19 Found:=469.21 (M+1).

### Example 295

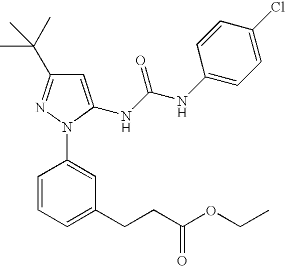

Example Z and p-chlorophenylisocyanate were reacted utilizing the same conditions as for Example 145 to yield ethyl 3-(3-(3-tert-butyl-5-(3-(4-chlorophenyl)ureido)-1H-pyrazol-1-yl)phenyl)propanoate. ¹H NMR (DMSO-d₆): δ 9.12 (s, 1H), 8.37 (s, 1H), 7.41-7.27 (m, 8H), 6.34 (s, 1H), 5.73 (s, 1H), 4.01 (q, J=7.2 Hz, 2H), 2.90 (t, J=5.2 Hz, 2H), 2.62 (t, J=5.6 Hz, 2H), 1.25 (s, 9H), 1.125 (t, J=7.2 Hz, 3H); MS(ESI): Exact Mass: 468.19 Found:=469.21 (M+1).

### Example 296

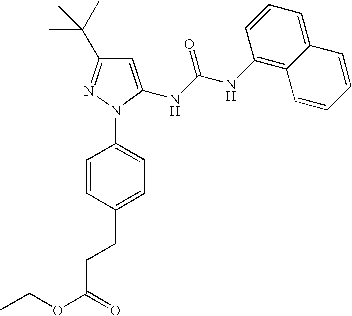

Example OOO and 1-naphthylisocyanate were reacted utilizing the same conditions as for Example 145 to yield ethyl 3-(4-(3-tert-butyl-5-(3-(naphthalen-1-yl)ureido)-1H-pyrazol-1-yl)phenyl)propanoate. δ 7.88-9.95 (m, 13H), 6.27 (s, 1H), 4.04 (q, J=7.2 Hz, 2H), 2.75 (t, J=5.2 Hz, 2H), 2.42 (t, J=5.6 Hz, 2H), 1.27 (s, 9H), 1.20 (t, J=7.2 Hz, 3H); MS(ESI): Exact Mass: 484.25 Found:=485.26 (M+1).

### Example 297

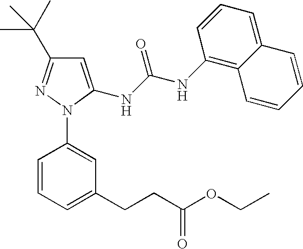

Example Z and 1-naphthylisocyanate were reacted utilizing the same conditions as for Example 145 to yield ethyl 3-(3-(3-tert-butyl-5-(3-(naphthalen-1-yl)ureido)-1H-pyrazol-1-yl)phenyl)propanoate. ¹H NMR (DMSO-d₆): δ 9.01 (s, 1H), 8.80 (s, 1H), 8.0-7.27 (m, 11H), 6.41 (s, 1H), 4.01 (q, J=7.2 Hz, 2H), 2.95 (t, J=5.2 Hz, 2H), 2.72 (t, J=5.6 Hz, 2H), 1.27 (s, 9H), 1.15 (t, J=7.2 Hz, 3H); MS(ESI): Exact Mass: 484.25 Found:=485.26 (M+1).

### Example 298

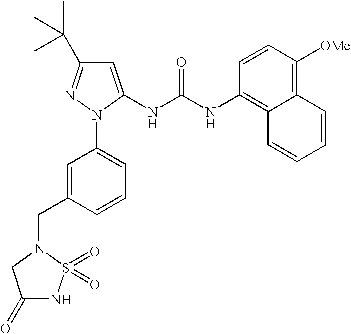

Example CC, 1-(4-methoxynaphthyl)isocyanate and Example DD were combined utilizing the same general approach for Example 162 to yield 1-(5-t-butyl-2-{3-[5-1,1,4-trioxo-1λ⁶-[1,2,5]thiadiazolidin-2-ylmethyl]-phenyl}-2H-pyrazol-3-yl)-1-(4-methoxynaphthyl)urea. ¹H NMR (DMSO-d₆): δ 8.69 (s, 1H), 8.61 (s, 1H), 8.15-6.90 (m, 10H), 6.36 (s, 1H), 4.37 (s, 2H), 3.93 (s, 3H), 1.22 (s, 9H); MS (ESI) Exact Mass: 562.20 Found:=563.2.

### Example PPP

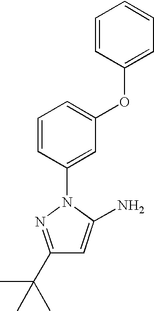

In a 250 mL Erlenmeyer flask with a magnetic stir bar, 3-phenoxyphenylamine (4.81 g, 0.026 mol) was added to 6 N HCl (40 mL) and cooled with an ice bath to 0° C. A solution of NaNO₂ (2.11 g, 0.0306 mol, 1.18 eq.) in water (5 mL) was added drop wise. After 30 min, SnCl₂.2H₂O (52.0 g, 0.23 mol, 8.86 eq.) in 6 N HCl (100 mL) was added and the reaction mixture was allowed to stir for 3 h, and then subsequently transferred to a 500 mL round bottom flask. To this, 4,4-dimethyl-3-oxopentanenitrile (3.25 g, 0.026 mol) and EtOH (100 ml) were added and the mixture refluxed for 4 h, concentrated in vacuo and the residue extracted with EtOAc (2×100 mL) and purified by column chromatography using hexane/EtOAc/Et₃N (8:2:0.2) to yield 3-tert-butyl-1-(3-phenoxyphenyl)-1H-pyrazol-5-amine (1.40 g, 17%). mp: 108-110° C.; ¹H NMR (CDCl₃): δ 7.3 (m, 10H), 5.7 (s, 1H), 4.9 (brs, 2H), 1.3 (s, 9H).

### Example 299

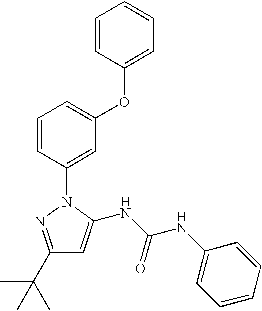

In a dry vial with a magnetic stir bar, Example PPP (0.184 g; 0.60 mmol) was dissolved in 2 mL CH₂Cl₂ (anhydrous) followed by the addition of phenylisocyanate (0.0653 mL; 0.60 mmol; 1 eq.). The reaction was kept under Ar and stirred for 18 h. Evaporation of solvent gave a crystalline mass that was recrystallized from EtOAc/hexane and then filtered washing with hexane/EtOAc (4:1) to yield 1-[3-tert-, butyl-1-(3-phenoxyphenyl)-1H-pyrazol-5-yl]-3-phenylurea (0.150 g, 50%). HPLC purity: 96%; ¹H NMR (CDCl₃): δ 7.5 (m, 16H), 6.8 (s, 1H), 6.5 (s, 1H), 1.4 (s, 9H).

### Example QQQ

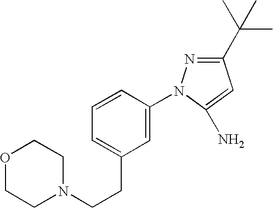

To a stirred solution of Example L (1.2 g, 3.5 mmol) in THF (6 ml) was added borane-methylsulfide (9 mmol). The mixture was heated to reflux for 90 min and cooled to RT, and 6 N HCl was added and heated to reflux for 10 min. The mixture was basified by adding sodium hydroxide, followed by extraction with ethyl acetate. The organic layer was dried (Na₂SO₄) filtered and concentrated in vacuo to yield 3-tert-butyl-1-[3-(2-morpholinoethyl)phenyl]-1H-pyrazol-5-amine (0.78 g), which was used without further purification.

### Example 300

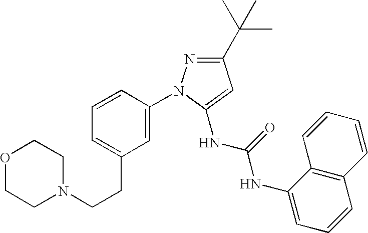

A mixture of Example QQQ (0.35 g, 1.07 mmol) and 1-naphthylisocyanate (0.18 g, 1.05 mmol) in dry CH₂Cl₂ (4 ml) was stirred at RT under N₂ for 18 h. The solvent was removed in vacuo and the crude product was purified by column chromatography using 5% methanol in CH₂Cl₂ (with a small amount of TEA) as the eluent (0.18 g, off-white solid) to yield 1-{3-tert-butyl-1-[3-(2-morpholinoethyl)phenyl]-1H-pyrazol-5-yl}-3-naphthalen-1-yl)urea. mp: 88-90° C.; ¹H NMR (200 MHz, DMSO-d₆): δ 9.07 (s, 1H), 8.80 (s, 1H), 8.06-7.92 (m, 3H), 7.69-7.44 (m, 7H), 7.40-7.29 (m, 1H), 6.44 (s, 1H), 3.57-3.55 (m, 4H), 3.33-3.11 (m, 4H), 2.40-2.38 (m, 4H), 1.32 (s, 9H); MS

### Example 301

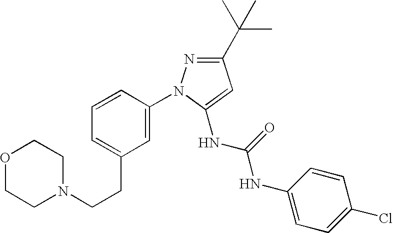

The title compound was synthesized in a manner analogous to Example 23 utilizing Example QQQ (0.35 g, 1.07 mmol) and 4-chlorophenylisocyanate (0.165 g, 1.05 mmol) to yield 1-{3-tert-butyl-1-[3-(2-morpholinoethyl)phenyl]-1H-pyrazol-5-yl}-3-(4-chlorophenyl)urea. mp: 82-84° C.; ¹H NMR (200 MHz, DMSO-d₆): δ 9.18 (s, 1H, s), 8.40 (s, 1H), 7.53-7.26 (m, 8H), 6.37 (s, 1H), 3.62-3.54 (m, 4H), 2.82-2.78 (m, 4H), 2.41-2.39 (m, 4H), 1.30 (s, 9H); MS

### Example A1

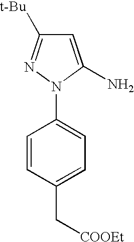

To a suspension of (4-amino-phenyl)-acetic acid (20 g, 0.13 mol) in 150 mL of concentrated HCl was added dropwise a solution of sodium nitrite (13.8 g, 0.2 mol) in water at 0° C. The mixture was stirred for 1 h, after which a solution of SnCl₂ (67 g, 0.3 mol) in concentrated HCl was added dropwise at such a rate that the reaction mixture never rose above 5° C. The resulted mixture was stirred for 2 h. The precipitate was collected by suction, washed with ethyl ether to afford 17 g of (4-hydrazino-phenyl)-acetic acid hydrochloride. MS (ESI) m/z: 167 (M+H⁺)

A solution of (4-hydrazino-phenyl)-acetic acid hydrochloride (17 g, 84 mmol) and 4,4-dimethyl-3-oxo-pentanenitrile (12.5 g, 0.1 mol) in EtOH (100 mL) containing concentrated HCl (25 mL) was heated to reflux overnight. After removal of the solvent, the residue was washed with Et₂O to afford 22 g of [4-(5-amino-3-t-butyl-pyrazol-1-yl)-phenyl]-acetic acid hydrochloride. MS (ESI) m/z: 274 (M+H⁺).

To a solution of [4-(5-amino-3-t-butyl-pyrazol-1-yl)-phenyl]-acetic acid hydrochloride (22 g, 71 mmol) in EtOH (250 mL) cooled in an ice-water bath was added dropwise SOCl₂ (40 mL). The mixture was heated to reflux for 2 h. After removal of the solvent, the residue was washed with Et₂O to afford 22.5 g of ethyl 2-(4-(3-t-butyl-5-amino-1H-pyrazol-1-yl)phenyl)acetate. ¹H NMR (300 MHz, DMSO-d₆), δ7.55-7.45 (m, 4H), 5.61 (s, 1H), 4.08 (q, J=6.9 Hz, 2H), 3.77 (s, 2H), 1.27 (s, 9H), 1.19 (t, J=6.9 Hz, 3H); MS (ESI) m/z: 302 (M+H⁺)

### Example A2

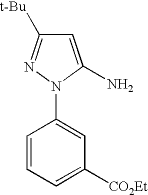

To a solution (200 mL) of m-amino benzoic acid (200 g, 1.46 mol) in concentrated HCl was added an aqueous solution (250 mL) of NaNO₂ (102 g, 1.46 mol) at 0° C. The reaction mixture was stirred for 1 h and a solution of SnCl₂.2H₂O (662 g, 2.92 mol) in concentrated HCl (2 L) was then added at 0° C., and the reaction stirred for an additional 2 h at RT. The precipitate was filtered and washed with ethanol and ether to yield 3-hydrazino-benzoic acid hydrochloride as a white solid.

The crude material from the previous reaction (200 g, 1.06 mol) and 4,4-dimethyl-3-oxo-pentanenitrile (146 g, 1.167 mol) in ethanol (2 L) were heated to reflux overnight. The reaction solution was evaporated in vacuo and the residue purified by column chromatography to yield ethyl 3-(3-t-butyl-5-amino-1H-pyrazol-1-yl)benzoate (Example A, 116 g, 40%) as a white solid together with 3-(5-amino-3-t-butyl-1H-pyrazol-1-yl)benzoic acid (93 g, 36%). ¹H NMR (DMSO-d₆): 8.09 (s, 1H), 8.05 (brd, J=8.0 Hz, 1H), 7.87 (brd, J=8.0 Hz, 1H), 7.71 (t, J=8.0 Hz, 1H), 5.64 (s, 1H), 4.35 (q, J=7.2 Hz, 2H), 1.34 (t, J=7.2 Hz, 3H), 1.28 (s, 9H).

### Example 302

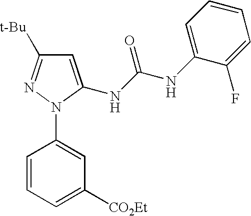

To a solution of Example A2 (143 mg, 0.5 mmol) and triethylamine (143 mg, 0.5 mmol) in anhydrous THF (5 mL) was added 1-fluoro-2-isocyanato-benzene (67 mg, 0.5 mmol) at 0° C. The mixture was stirred at RT for 3 h, then poured into water (10 mL), and extracted with CH₂Cl₂. The combined organic extracts were washed with brine, dried (Na₂SO₄), filtered, concentrated and purified via preparative-TLC to afford ethyl 3-{3-t-butyl-5-[3-(2-fluorophenyl)ureido]-1H-pyrazol-1-yl}benzoate (40 mg, 19% yield).

### Example 303

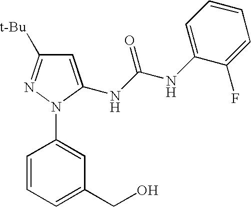

To a stirred solution of Example 302 (35 mg, 0.083 mmol) in THF (5 mL) was added LAH powder (7 mg, 0.18 mmol) by portion at 0° C. under N₂. The mixture was stirred at RT for 2 h, then quenched with water, and extracted with EtOAc. The combined organic extracts were washed with brine, dried (Na₂SO₄), filtered, concentrated and purified via preparative-TLC to afford 1-{3-t-butyl-1-[3-(hydroxymethyl)phenyl]-1H-pyrazol-5-yl}-3-(2-fluorophenyl)urea (20 mg, 63% yield).

### Example 304

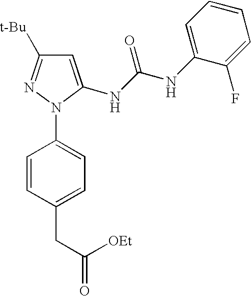

To a solution of Example A1 (150 mg, 0.5 mmol) and triethylamine (101 mg, 1.0 mmol) in anhydrous THF (5 mL) was added 1-fluoro-2-isocyanato-benzene (68 mg, 0.5 mmol) at 0° C. The mixture was stirred at RT for 3 h before, then poured into water (50 mL), and extracted with CH₂Cl₂ (3×50 mL). The combined organic layers were washed with brine, dried (Na2SO4), filtered and concentrated to a solid, which was purified by column chromatography to afford 2-(4-(3-t-butyl-5-(3-(2-fluorophenyl)ureido)-1H-pyrazol-1-yl)-phenyl)acetate (140 mg, 64% yield).

### Example 305

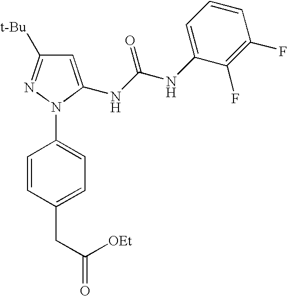

A solution of Example A1 (300 mg, 1.0 mmol), triethylamine (202 mg, 2.0 mmol) and CDI (162 mg, 1.0 mmol) in DMF (5.0 mL) was stirred at RT for 6 h. The mixture was added 2,3-difluoro-phenylamine (129 mg, 1.0 mmol), stirred for 5 h, poured into water (50 mL) and extracted with CH₂Cl₂ (3×50 mL). The combined organic layers were washed with 1.0 N of HCl, brine, dried (Na₂SO₄), filtered and concentrated to a solid, which was purified by column chromatography to afford ethyl 2-(4-(3-t-butyl-5-(3-(2,3-difluorophenyl)ureido)-1H-pyrazol-1-yl)phenyl)acetate (220 mg, 48% yield).

### Example 306

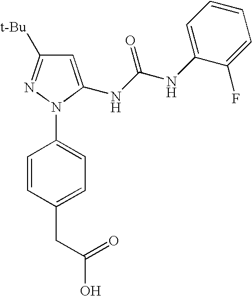

A mixture of Example 305 (100 mg, 0.22 mmol) in an aqueous solution of LiOH (2 N, 5 mL) and THF (10 mL) was stirred overnight at RT. After removal of the organic solvent, the mixture was extracted with Et₂O. The aqueous layer was then acidified with 2 N HCl to pH=4 and extracted with EtOH. The combined organic layers were washed with brine, dried (Na₂SO₄), filtered and concentrated to a solid and dried to give the crude product, which was purified by preparative HPLC to afford 2-(4-(3-t-butyl-5-(3-(2-fluorophenyl)ureido)-1H-pyrazol-1-yl)phenyl)acetic acid (50 mg, 61% yield). ¹H NMR (400 MHz, DMSO-d₆): δ 10.07 (br s, 1H), 9.92 (br s, 1H), 7.91 (t, J=5.7 Hz, 1H), 7.38 (d, J=5.7 Hz, 2H), 7.30 (d, J=5.7 Hz, 2H), 7.14 (t, J=5.4 Hz, 1H), 7.05 (t, J=5.4 Hz, 1H), 6.96 (m, 1H), 6.25 (s, 1H), 3.26 (s, 2H), 1.24 (s, 9H); MS (ESI) m/z: 411 (M+H⁺).

### Example 307

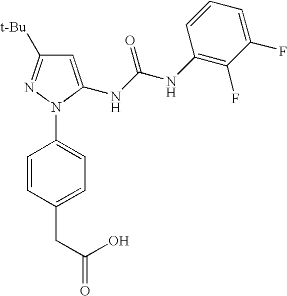

Using the same procedure as for Example 306, Example 307 (100 mg, 0.22 mmol) was transformed to afford 2-(4-(3-t-butyl-5-(3-(2,3-difluorophenyl)ureido)-1H-pyrazol-1-yl)-phenyl)acetic acid (50 mg, 53% yield). ¹H NMR (400 MHz, DMSO-d₆): δ 10.75 (br s, 1H), 7.62 (t, J=7.8 Hz, 1H), 7.43 (d, J=6.0 Hz, 2H), 7.28 (d, J=6.0 Hz, 2H), 7.04-6.95 (m, 2H), 6.22 (s, 1H), 3.28 (s, 2H), 1.24 (s, 9H); MS (ESI) m/z: 429 (M+H⁺).

### Example A3

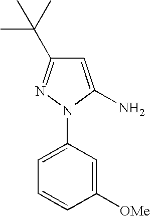

To a solution of 3-methoxyphenylhydrazine hydrochloride (1.0 g, 5.7 mmol) in toluene (5 mL) was added pivaloylacetonitrile (0.70 g, 5.5 mmol). The reaction mixture was heated to reflux for 5 h, filtered and washed with hexane to obtain 3-t-butyl-1-(3-methoxyphenyl)-1H-pyrazol-5-amine (1.22 g, 89% yield) as its hydrochloride salt as an pale yellow solid which was used without further purification. ¹H NMR (CDCl₃): δ 7.35 (t, J=8.4 Hz, 1H), 7.04 (t, J=2.1 Hz, 1H), 7.00 (dd, J=1.5 and 7.5 Hz, 1H), 6.95 (dd, J=2.1 and 8.4 Hz, 1H), 5.90 (bs, 2H), 5.83 (s, 1H), 3.81 (s, 3H), 1.89 (s, 9H); MS (EI) m/z: 246 (M+H⁺).

### Example 308

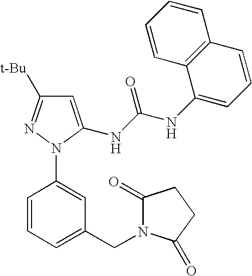

To a mixture of Example A36 (100 mg, 0.23 mmol), K₂CO₃ (64 mg, 0.46 mmol) and KI (10 mg) in DMF (2 mL) was added pyrrolidine-2,5-dione (23 mg, 0.23 mmol) at RT. The resulting mixture was stirred overnight, concentrated and purified by column chromatography to yield 1-(3-t-butyl-1-{3-[(2,5-dioxopyrrolidin-1-yl)methyl]phenyl}-1H-pyrazol-5-yl)-3-(naphthalen-1-yl)urea (50 mg, 44% yield). ¹H-NMR (300 MHz, DMSO-d₆): δ 9.00 (s, 1. H), 8.86 (s, 1H), 8.02 (d, J=8.1 Hz, 1H), 7.89-7.92 (m, 2H), 7.63 (d, J=7.8 Hz, 1H), 7.42-7.55 (m, 6H), 7.29 (m, 1H), 6.40 (s, 1H), 4.62 (s, 2H), 2.63 (s, 2H), 1.27 (s, 9H).

### Example 309

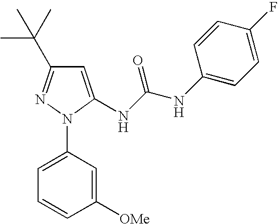

Using the same procedure as for Example 308, Example A3 (70 mg, 0.29 mmol) and 4-fluorophenylisocyanate (39 mg, 0.29 mmol) were combined to afford 1-(3-t-butyl-1-(3-methoxyphenyl)-1H-pyrazol-5-yl)-3-(4-fluorophenyl)urea as a white powder (38 mg, 35% Yield). ¹H NMR (CDCl₃): δ 7.59 (bs, 1H), 7.16 (t, J=8.4 Hz, 1H), 6.8-7.1 (m, 8H), 6.77 (dd, J=1.8 and 8.7 Hz, 1H), 6.30 (s, 1H), 3.66 (s, 3H), 1.27 (s, 9H); MS (EI) m/z: 383 (M+H⁺).

### Example 310

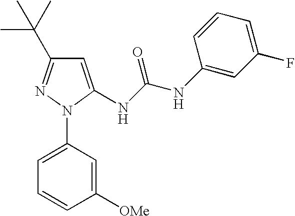

Using the same procedure as for Example 308, Example A3 (60 mg, 0.21 mmol) and 3-fluorophenyl isocyanate (29 mg, 0.21 mmol) were combined to afford 1-(3-t-butyl-1-(3-methoxyphenyl)-1H-pyrazol-5-yl)-3-(3-fluorophenyl)urea (49 mg, 60% yield). ¹H NMR (CDCl₃): δ 7.2-7.3 (m, 3H), 7.17 (bs, 1H), 6.95-7.05 (m, 2H), 6.93 (dd, J=1.6, and 8.2 Hz, 1H), 6.87 (dd, J=1.8, and 7.6 Hz, 1H), 6.79 (dt, J=1.9, and 8.8 Hz, 1H), 6.64 (s, 1H), 6.39 (s, 1H), 3.77 (s, 3H), 1.35 (s, 9H);MS (EI) m/z: 383 (M+H⁺).

### Example 311

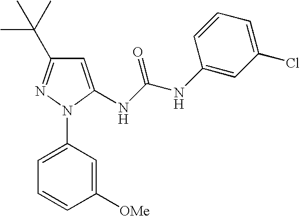

Using the same procedure as for Example 308, Example A3 (70 mg, 0.29 mmol) and 3-chlorophenylisocyanate (44 mg, 0.29 mmol) were combined to afford 1-(3-t-butyl-1-(3-methoxyphenyl)-1H-pyrazol-5-yl)-3-(3-chlorophenyl)urea (83 mg, 73% yield). ¹H NMR (CDCl₃): δ 8.30 (s, 1H), 7.38 (s, 1H), 7.20 (t, J=1.8 Hz, 1H), 7.07 (m, 2H), 6.95 (dt, J=1.2, and 7.8 Hz, 2H), 6.82 (t, J=2.1 Hz, 1H), 6.78 (s, 1H), 7.72 (dd, J=2.1, and 8.7 Hz, 1H), 6.28 (s, 1H), 3.56 (s, 3H), 1.21 (s, 9H); MS (EI) m/z: 399 (M+H⁺).

### Example 312

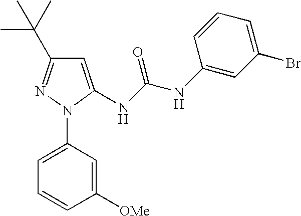

Using the same procedure as for Example 308, Example A3 (70 mg, 0.29 mmol) and 3-bromophenylisocyanate (57 mg, 0.29 mmol) were combined to afford 1-(3-t-butyl-1-(3-methoxyphenyl)-1H-pyrazol-5-yl)-3-(3-bromophenyl)urea as a white solid (107 mg, 85% yield). ¹H NMR (CDCl₃): δ 8.08 (bs, 1H), 7.38 (s, 1H), 7.23 (s, 1H). 7.0-7.2 (m, 4H), 7.8-7.9 (m, 2H), 6.75 (dd, J=2.4 and 8.4 Hz, 1H), 6.32 (s, 1H), 3.59 (s, 3H), 1.24 (s, 9H); MS (EI) m/z: 443 and 445 (M+ and M⁺+2).

### Example 313

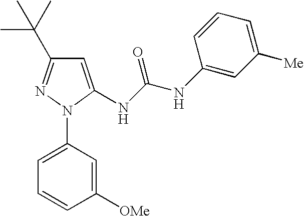

Using the same procedure as for Example 308, Example A3 (70 mg, 0.29 mmol) and 3-methylphenylisocyanate (38 mg, 0.29 mmol) were combined to afford 1-(3-t-butyl-1-(3-methoxyphenyl)-1H-pyrazol-5-yl)-3-m-tolylurea as a white solid (107 mg, 98% Yield). ¹H NMR (CDCl₃): δ 7.88 (bs, 1H), 7.34 (s, 1H), 7.0-7.2 (m, 2H), 6.95 (s, 1H), 6.8-6.94 (m, 4H). 6.73 (dd, J=2.4 and 8.4 Hz, 1H), 6.30 (s, 1H), 3.58 (s, 3H), 2.19 (s, 3H), 1.25 (s, 9H); MS (EI) m/z: 379 (M+H⁺).

### Example 314

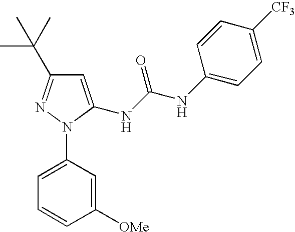

Using the same procedure as for Example 308, Example A3 (70 mg, 0.29 mmol)) and 4-(trifluoromethyl)phenyl isocyanate (53 mg, 0.29 mmol) were combined to afford 1-(3-t-butyl-1-(3-methoxyphenyl)-1H-pyrazol-5-yl)-3-(4-(trifluoromethyl)phenyl)urea (73 mg, 59% yield). ¹H NMR (CDCl₃): δ 8.50 (s, 1H), 7.44 (AB quartet, J=8.7 Hz, 2H), 7.33 (s, 1H), 7.27 (AB quartet, J=8.7 Hz, 2H), 7.06(t, J=7.8 Hz, 1H), 6.7-6.9 (m, 3H), 6.34 (s, 1H), 3.54 (s, 3H), 1.22 (s, 9H); MS (EI) m/z: 433 (M+H⁺).

### Example 315

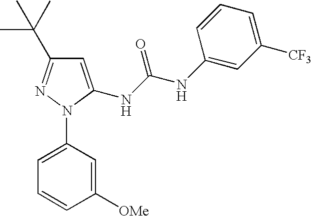

Using the same procedure as for Example 308, Example A3 (50 mg, 0.20 mmol) and 4-(trifluoromethyl)phenyl isocyanate (30 mmg, 0.20 mmol) were combined to afford 1-(3-t-butyl-1-(3-methoxyphenyl)-1H-pyrazol-5-yl)-3-(3-(trifluoromethyl)phenyl)urea (30 mg, 39% yield). ¹H NMR (CDCl₃): δ 8.14 (s, 1H), 7.51 (s, 1H), 7.38 (d, J=8.1 Hz, 1H), 7.32 (t, J=8.0 Hz, 1H), 7.27 (d, J=7.6 Hz, 1H), 7.1-7.2 (m, 2H), 6.88 (t, J=2.0 Hz, 1H), 6.84 (dd, J=1.0 Hz, and 7.8 Hz, 1H), 6.79 (dd, J=2.4, and 7.8 Hz, 1H), 6.38 (s, 1H), 3.61 (s, 3H), 1.27 (s, 9H); MS (EI) m/z: 433 (M+H⁺).

### Example 316

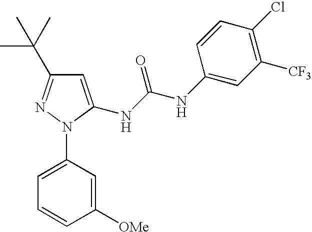

Using the same procedure as for Example 308, Example A3 (70 mg, 0.29 mmol) and 3-chloro-4-(trifluoromethyl)phenyl isocyanate (63 mg, 0.29 mmol) were combined to afford 1-(3-t-butyl-1-(3-methoxyphenyl)-1H-pyrazol-5-yl)-3-(4-chloro-3-(trifluoromethyl)phenyl)urea as a white solid (49 mg, 37% Yield). ¹H NMR (CDCl₃): δ 8.48 (s, 1H), 7.52 (d, J=2.1 Hz, 1H), 7.38 (dd, J=2.1, 8.7 Hz, 1H), 6.79 (bs, 2H), 6.76 (s, 1H), 6.37 (s, 1H), 3.58 (s, 3H), 1.22 (s, 9H); MS (EI) m/z: 467 (M+H⁺).

### Example 317

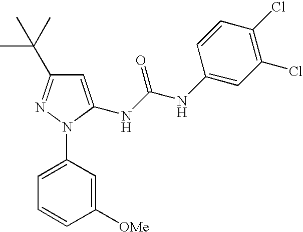

Using the same procedure as for Example 308, Example A3 (70 mg, 0.29 mmol) and 3,4-dichlorophenyl isocyanate (54 mg, 0.29 mmol) were combined to afford 1-(3-t-butyl-1-(3-methoxyphenyl)-1H-pyrazol-5-yl)-3-(3,4-dichlorophenyl)urea (38 mg, 31% yield). ¹H NMR (CDCl₃): δ 8.13 (s, 1H), 7.35 (d, J=2.4 Hz, 1H), 7.24 (dd, J=0.6, and 3.3 Hz, 1H), 7.19 (s, 1H), 7.12 (t, J=8.1 Hz, 1H), 6.96 (dd, J=2.4, and 8.7 Hz, 1H), 6.7-6.9 (m, 3H), 6.37 (s, 1H), 3.62 (s, 3H), 1.24 (s, 9H); MS (EI) m/z: 433 (M+H⁺).

### Example 318

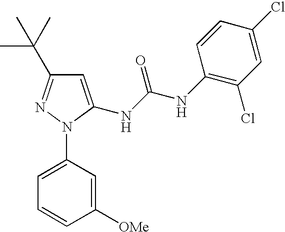

Using the same procedure as for Example 308, Example A3 (70 mg, 0.29 mmol) and 2,4-dichlorophenyl isocyanate (54 mg, 0.29 mmol) were combined to afford 1-(3-t-butyl-1-(3-methoxyphenyl)-1H-pyrazol-5-yl)-3-(2,4-dichlorophenyl)urea (76 mg, 61% yield). ¹H NMR (CDCl₃): δ 7.96 (d, J=9.0 Hz), 7.67 (s, 1H), 7.65 (s, 1H), 7.29 (d, J=2.4 Hz, 1H), 7.19 (t, J=7.8 Hz, 1H), 7.14 (dd, J=2.4, and 9.0 Hz, 1H), 6.9-7.0 (m, 2H), 6.78 (dd, J=2.4, and 8.7 Hz, 1H), 6.33 (s, 1H), 3.70 (s, 3H), 1.32 (s, 9H); MS (EI) m/z: 433 (M+H⁺).

### Example 319

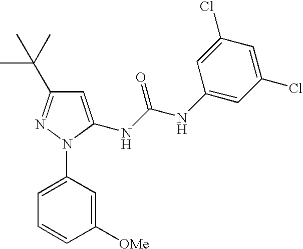

Using the same procedure as for Example 308, Example A3 (70 mg, 0.29 mmol) and 3,5-dichlorophenyl isocyanate (54 mg, 0.29 mmol) were combined to afford 1-(3-t-butyl-1-(3-methoxyphenyl)-1H-pyrazol-5-yl)-3-(3,5-dichlorophenyl)urea (59 mg, 48% yield). ¹H NMR (CDCl₃): δ 7.73 (s, 1H), 7.1-7.3 (m, 3H), 7.03 (t, J=1.8 Hz, 1H), 6.9-7.0 (m, 3H), 6.84 (dd, J=1.8, and 7.5 Hz, 1H), 6.40 (s, 1H), 3.71 (s, 3H), 1.30 (s, 9H); MS (EI) m/z: 433 (M+H⁺).

### Example 320

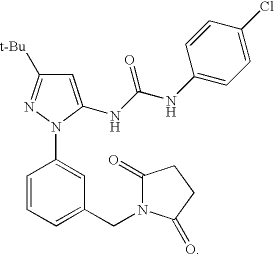

Using the same procedure as for Example 308, Example A37 (100.0 mg, 0.25 mmol) was transformed to afford 1-(3-t-butyl-1-{3-[(2,5-dioxopyrrolidin-1-yl)methyl]phenyl}-1H-pyrazol-5-yl)-3-(4-chlorophenyl)urea (35 mg, 29% yield). ¹H NMR (300 MHz, DMSO-d₆): δ 9.01 (s, 1H), 8.46 (s, 1H), 7.35-7.45 (m, 5H), 7.25-7.30 (m, 2H), 6.34 (s, 1H), 4.60 (s, 2H), 2.64 (s, 2H), 1.27 (s, 9H).

### Example 321

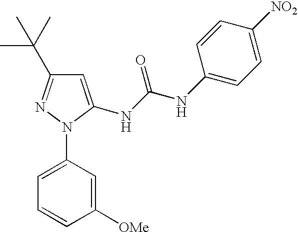

Using the same procedure as for Example 308, Example A3 (70 mg, 0.29 mmol) and 4-nitrophenylisocyanate (47 mg, 0.29 mmol) were combined to afford 1-(3-t-butyl-1-(3-methoxyphenyl)-1H-pyrazol-5-yl)-3-(4-nitrophenyl)urea (62 mg, 53% yield). ¹H NMR (CDCl₃): δ 8.54 (s, 1H), 8.08 (AB quartet, J=9.0 Hz, 2H), 7.45 (AB quartet, J=9.0 Hz, 2H), 7.38 (s, 1H), 7.11 (t, J=8.1 Hz, 1H), 6.7-6.9 (m, 3H), 6.45 (s, 1H), 3.61 (s, 3H), 1.26 (s, 9H); MS (EI) m/z: 410 (M+H⁺).

### Example 322

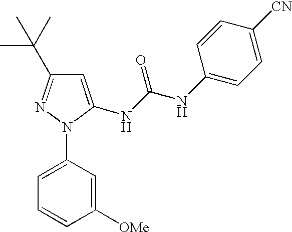

Using the same procedure as for Example 308, Example A3 (70 mg, 0.29 mmol) and 4-cyanophenyl isocyanate (41 mg, 0.29 mmol) were combined to afford 1-(3-t-butyl-1-(3-methoxyphenyl)-1H-pyrazol-5-yl)-3-(4-cyanophenyl)urea (79 mg, 71% yield). ¹H-NMR (CDCl₃): δ 8.70 (s, 1H), 7.47 (AB quartet, J=8.7 Hz, 2H), 7.40 (AB quartet, J=8.7 Hz, 2H), 7.37 (s, 1H), 7.11 (t, J=7.8 Hz, 1H), 6.7-6.9 (m, 3H), 6.42 (s, 1H), 3.59 (s, 3H), 1.24 (s, 9H); MS (EI) m/z: 390 (M+H⁺).

### Example 323

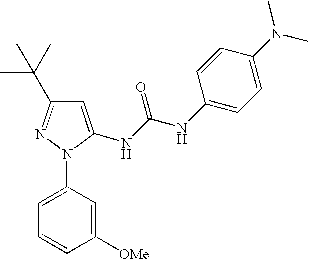

Using the same procedure as for Example 308, Example A3 (70 mg, 0.29 mmol) and 4-(N,N-dimethylamino)phenyl isocyanate (46 mg, 0.29 mmol) were combined to afford 1-(3-t-butyl-1-(3-methoxyphenyl)-1H-pyrazol-5-yl)-3-(4-(dimethylamino)phenyl)urea as a brown oil (25 mg, 21% Yield). ¹H NMR (CDCl₃): δ 7.19 (t, J=8.1 Hz, 1H), 7.01 (AB quartet, J=9.0 Hz, 2H), 6.85-6.95 (m, 3H), 7.47 (dd, J=2.1, and 8.1 Hz, 1H), 6.60 (AB quartet, J=9.0 Hz, 2H), 6.40 (s, 1H), 3.73 (s, 3H), 2.92 (s, 6H), 1.32 (s, 9H); MS (EI) m/z: 408 (M+H⁺).

### Example 324

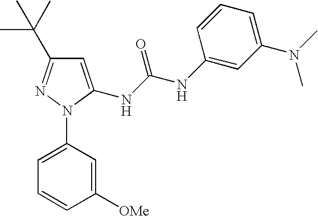

Using the same procedure as for Example 308, Example A3 (62 mg, 0.25 mmol) and 3-(N,N-dimethylamino)phenyl isocyanate (52 mg, 0.32 mmol) were combined to afford 1-(3-t-butyl-1-(3-methoxyphenyl)-1H-pyrazol-5-yl)-3-(3-(dimethylamino)phenyl)urea (11 mg, 11% yield). ¹H NMR (CDCl₃): δ 7.24 (t, J=8.2 Hz, 1H), 7.11 (t, J=8.1 Hz, 1H), 6.9-7.0 (m, 4H), 6.83 (m, 1H), 6.66 (bs, 1H), 6.48 (dt, J=2.4, and 8.2 Hz, 2H), 6.41 (s, 1H), 3.74 (s, 3H), 2.89 (s, 6H), 1.34 (s, 9H); MS (EI) m/z: 408 (M+H⁺).

### Example 325

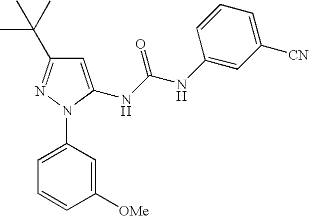

Using the same procedure as for Example 308, Example A3 (45 mg, 0.18 mmol) and 3-cyanophenyl isocyanate (26 mg, 0.18 mmol) were combined to afford 1-(3-t-butyl-1-(3-methoxyphenyl)-1H-pyrazol-5-yl)-3-(3-cyanophenyl)urea (35 mg, 50% yield). ¹H NMR (CDCl₃): δ 8.14 (s, 1H), 7.61 (s, 1H), 7.52 (m, 1H), 7.35 (t, J=8.0 Hz, 1H), 7.29 (d, J=6.9 Hz, 1H), 7.21 (d, J=8.0 Hz, 1H), 7.18 (s, 1H), 6.90 (s, 1H), 6.88 (d, J=7.6 Hz, 1H), 6.80 (dd, J=2.4, and 7.6 Hz, 1H), 6.42 (s, 1H), 3.67 (s, 3H), 1.30 (s, 9H); MS (EI) m/z: 390 (M+H⁺).

### Example 326

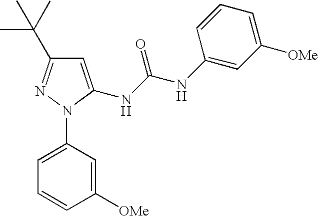

Using the same procedure as for Example 308, Example A3 (45 mg, 0.18 mmol) and 3-methoxyphenyl isocyanate (26 mg, 0.18 mmol) were combined to afford 1-(3-t-butyl-1-(3-methoxyphenyl)-1H-pyrazol-5-yl)-3-(3-methoxyphenyl)urea (17 mg, 24% yield). ¹H NMR (CDCl₃): δ 7.28 (s, 1H), 7.24 (t, J=8.0 Hz, 1H), 7.15 (t, J=8.2 Hz, 1H), 6.9-7.0 (m, 4H), 6.83(dd, J=2.3, and 8.7 Hz, 1H), 6.71 (dd, J=1.6, and 8.0 Hz, 1H), 6.64 (dd, J=2.4, and 8.2 Hz, 1H), 6.39 (s, 1H), 3.74 (s, 3H), 3.72 (s, 3H), 1.33 (s, 9H); MS (EI) m/z: 395 (M+H⁺).

### Example 327

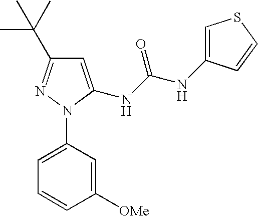

Using the same procedure as for Example 308, Example A3 (70 mg, 0.29 mmol) and 3-thienyl isocyanate (36 mg, 0.29 mmol) were combined to afford 1-(3-t-butyl-1-(3-methoxyphenyl)-1H-pyrazol-5-yl)-3-(thiophen-3-yl)urea (45 mg, 43% yield). ¹H NMR (CDCl₃): δ 7.05-7.3 (m, 4H), 6.8-7.0 (m, 4H), 6.76 (s, 1H), 6.40 (s, 1H), 3.76 (s, 3H), 1.35 (s, 9H); MS (EI) m/z: 371 (M+H⁺).

### Example 328

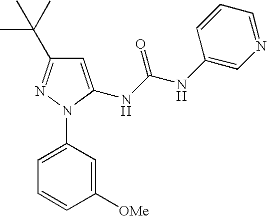

Using the same procedure as for Example 308, Example A3 (86 mg, 0.35 mmol) and 3-pyridinylisocyanate (51 mg, 0.43 mmol) were combined to afford 1-(3-t-butyl-1-(3-methoxyphenyl)-1H-pyrazol-5-yl)-3-(pyridin-3-yl)urea as a white solid (89 mg, 69% yield). ¹H NMR (DMSO-d₆): δ 10.0 (bs, 1H), 8.92 (bs, 1H), 8.87 (s, 1H), 8.39 (d, J=5.2 Hz, 1H), 8.17 (d, J=8.4 Hz, 1H), 7.70 (dd, J=5.1, and 8.1 Hz, 1H), 7.41 (t, J=8.2 Hz, 1H), 7.0-7.1 (m, 2H), 6.96 (dd, J=2.4, and 8.3 Hz, 1H), 6.38 (s, 1H), 3.80 (s, 3H), 1.29 (s, 9H); MS (EI) m/z: 366 (M+H⁺).

### Example 329

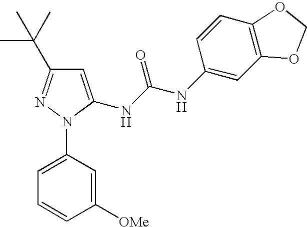

Using the same procedure as for Example 308, Example A3 (86 mg, 0.35 mmol) and 5-isocyanatobenzo[d][1,3]dioxole (69 mg, 0.43 mmol) were combined to afford 1-(benzo[d][1,3]dioxo-5-yl)-3-(3-t-butyl-1-(3-methoxyphenyl)-1H-pyrazol-5-yl)urea as a pale yellow solid (98 mg, 68% yield). ¹H NMR (DMSO-d₆): δ 8.94 (s, 1H), 8.92 (bs, 1H), 8.31 (s, 1H), 7.42 (t, J=8.1 Hz, 1H), 7.0-7.2 (m, 3H), 6.98 (dd, J=1.8, and 8.4 Hz, 1H), 6.80 (d, J=8.4 Hz, 1H), 6.71 (dd, J=2.0, and 8.4 Hz, 1H), 6.35 (s, 1H), 5.96 (s, 2H), 3.80 (s, 3H), 1.28 (s, 9H); MS (EI) m/z: 409 (M+H⁺).

### Example A4

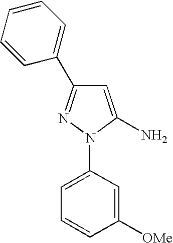

To a solution of 3-methoxyphenylhydrazine hydrochloride (0.6 g, 3.44 mmol) in toluene was added commercially available benzoyl acetonitrile (0.5 g, 3.44 mmol). The reaction mixture was heated to reflux overnight, filtered and washed with hexane to obtain 1-(3-methoxyphenyl)-3-phenyl-1H-pyrazol-5-amine (0.82 g, 79% yield) as a grey hydrochloride salt which was used without any further purification. ¹H NMR (DMSO-d₆): δ 7.78 (m, 2H), 7.2-7.6 (m, 6H), 6.97 (m, 1H), 6.01 (s, 1H), 3.81 (s, 3H), 1.27 (s, 9H); MS (EI) m/z: 266 (M+H⁺).

### Example 330

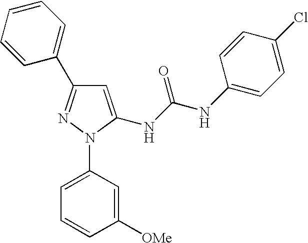

Using the same procedure as for Example 308, Example A4 (70 mg, 0.23 mmol) and 4-chlorophenylisocyanate (36 mg, 0.23 mmol) were combined to afford 1-(4-chlorophenyl)-(3-methoxyphenyl)-3-phenyl-1H-pyrazol-5-yl)urea (75 mg, 77% yield). ¹H NMR (DMSO-d₆): δ8.59 (s, 1H), 7.86 (d, J=1.6 Hz, 1H), 7.84 (s, 1H), 7.3-7.5 (m, 9H), 7.21 (s, 1H), 7.19 (d, J=1.6 Hz, 1H), 7.05 (dd, J=2.0, and 9.2 Hz, 1H), 6.94 (s, 1H), 3.83 (s, 3H); MS (EI) m/z: 419 (M+H⁺).

### Example 331

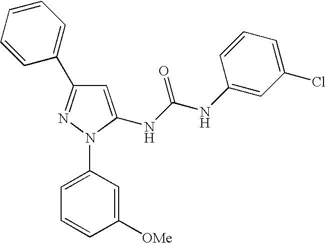

Using the same procedure as for Example 308, Example A4 (50 mg, 0.17 mmol) and 3-chlorophenylisocyanate (25 mg, 0.17 mmol) were combined to afford 1-(3-chlorophenyl)-(3-methoxyphenyl)-3-phenyl-1H-pyrazol-5-yl)urea (46 mg, 66% yield). ¹H NMR (CDCl₃): δ7.92 (s, 1H), 7.67 (dd, J=1.5, and 8.2 Hz, 2H), 7.54 (s, 1H), 7.25-7.4 (m, 3H), 7.15 (t, J=2.0 Hz, 1H), 7.09 (t, J=8.1 Hz, 1H), 7.02 (t, J=8.0 Hz, 1H), 6.8-7.0 (m, 3H), 6.83 (dd, J=1.2, and 7.8 Hz, 1H), 6.71 (dd, J=2.0, and 8.1 Hz, 1H), 6.64 (s, 1H), 3.57 (s, 3H); MS (EI) m/z: 419 (M+H⁺).

### Example 332

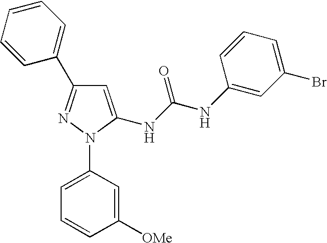

Using the same procedure as for Example 308, Example A4 (50 mg, 0.17 mmol) and 3-bromophenylisocyanate (25 mg, 0.17 mmol) were combined to afford 1-(3-bromophenyl)-(3-methoxyphenyl)-3-phenyl-1H-pyrazol-5-yl)urea (46 mg, 60% yield). ¹H NMR (DMSO-d₆): δ 9.28 (s, 1H), 8.62 (s, 1H), 7.85 (m, 3H), 7.0-7.5 (m, 10H), 6.95 (s, 1H), 3.83 (s, 3H); MS (EI) m/z: 463 and 465 (M+ and M⁺+2).

### Example 333

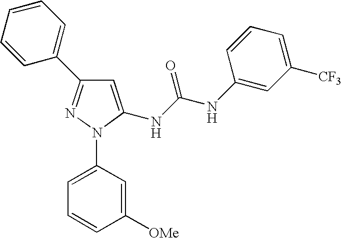

Using the same procedure as for Example 308, Example A4 (50 mg, 0.17 mmol) and 3-trifluoromethylphenyl isocyanate (31 mg, 0.17 mmol) were combined to afford 1-(1-(3-methoxyphenyl)-3-phenyl-1H-pyrazol-5-yl)-3-(3-trifluoromethyl)phenyl)urea (43 mg, 57% yield). ¹H NMR (DMSO-d₆): δ 9.45 (s, 1H), 8.67 (s, 1H), 8.00 (s, 1H), 7.87 (m, 2H), 7.0-7.6 (m, 10H), 6.97 (s, 1H), 3.83 (s, 3H); MS (EI) m/z: 453 (M+H⁺).

### Example 334

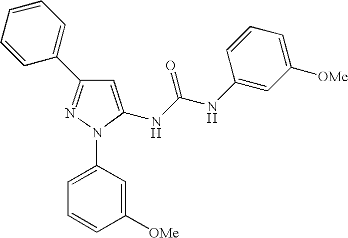

Using the same procedure as for Example 308, Example A4 (50 mg, 0.17 mmol) and 3-methoxyphenyl isocyanate (25 mg, 0.17 mmol) were combined to afford 1-(3-methoxyphenyl)-3-(1-(3-methoxyphenyl)-3-phenyl-1H-pyrazol-5-yl)urea (47 mg, 68% yield). ¹H NMR (DMSO-d₆): δ9.11 (s, 1H), 8.52 (s, 1H), 7.86 (d, J=1.3 Hz, 1H), 7.84 (s, 1H), 7.47 (t, J=7.8 Hz, 1H), 7.44 (t, J=7.8 Hz, 2H), 7.34 (t, J=7.3 Hz, 1H), 7.0-7.2 (m, 5H), 6.94 (s, 1H), 6.93 (m, 1H), 6.57 (dd, J=2.4, and 8.2 Hz, 1H), 3.83 (s, 3H), 3.72 (s, 3H); MS (EI) m/z: 415 (M+H⁺).

### Example 335

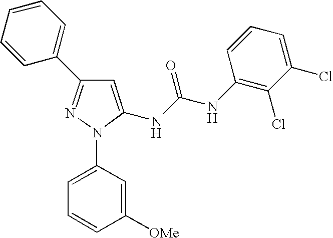

Using the same procedure as for Example 308, Example A4 (50 mg, 0.17 mmol) and 2,3-dichlorophenyl isocyanate (31 mg, 0.17 mmol) were combined to afford 1-(2,3-dichlorophenyl)-(3-methoxyphenyl)-3-phenyl-1H-pyrazol-5-yl)urea (41 mg, 55% yield). ¹H NMR (DMSO-d₆): δ 9.37 (s, 1H), 8.87 (s, 1H), 7.07 (dd, J=3.4, and 6.4 Hz, 1H), 7.86 (d, J=1.4 Hz, 1H), 7.84 (s, 1H), 7.50 (t, J=8.4 Hz, 1H), 7.44 (t, J=7.3 Hz, 2H), 7.2-7.4 (m, 5H), 7.06 (m, 1H), 6.95 (s, 1H), 3.84 (s, 3H); MS (EI) m/z: 453 (M+H⁺).

### Example A5

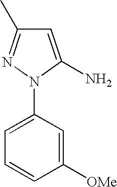

To a suspension of NaH (60%, 12.0 g, 0.3 mol) in THF (200 mL) was added dropwise acetic acid ethyl ester (17 g, 0.2 mol) and anhydrous acetonitrile (100 g, 0.24 mol) in THF (200 mL) at 80° C. The resulting mixture was refluxed overnight, and then cooled to RT. After removal of the volatiles in vacuo, the residue was diluted in EtOAc and aqueous 10% HCL. The combined organic extracts were washed with saturated NaHCO₃ and brine, then dried (MgSO₄), filtered, concentrated to yield 3-oxobutyronitrile (10 g), which was used for the next step reaction without further purification.

To a solution of 3-oxobutanenitrile (300 mg, 3.6 mmol) and 3-methoxyphenyl-hydrazine HCl (630 mg, 3.6 mmol) in absolute ethanol at RT was added conc. HCl (0.3 mL). The reaction mixture was stirred at 80° C. for 13 h. The solvent was evaporated under reduced pressure to obtain the crude product 1-(3-methoxyphenyl)-3-methyl-1H-pyrazol-5-amine as brown foam hydrochloride salt (690 mg, 80% yield), which was used without further purification. MS (EI) m/z: 204 (M+H⁺).

### Example 336

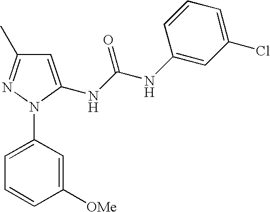

Using the same procedure as for Example 308, Example A5 (60 mg, 0.25 mmol) and 3-chlorophenyl isocyanate (38 mg, 0.25 mmol) were combined to afford 1-(3-chlorophenyl)-3-(1-(3-methoxyphenyl)-3-methyl-1H-pyrazol-5-yl)urea (15 mg, 17% yield). ¹H NMR (CDCl₃): δ7.97 (bs, 1H), 7.34 (bs, 1H), 7.30 (t, J=2.0 Hz, 1H), 7.0-7.25 (m, 4H), 6.85 (s, 1H), 6.84 (m, 1H), 6.79 (m, 1H), 6.30 (s, 1H), 3.67 (s, 3H), 2.22 (s, 3H); MS (EI) m/z: 357 (M+H⁺).

### Example 337

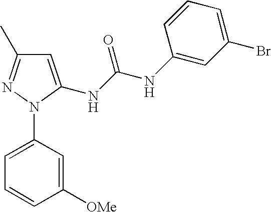

Using the same procedure as for Example 308, Example A5 (50 mg, 0.21 mmol) and 3-bromophenyl isocyanate (41 mg, 0.21 mmol) were combined to afford 1-(3-bromophenyl)-3-(1-(3-methoxyphenyl)-3-methyl-1H-pyrazol-5-yl)urea (12 mg, 15% yield). ¹H NMR (CDCl₃): δ 8.20 (bs, 1H), 7.51 (bs, 1H), 7.40 (m, 2H), 7.0-7.2 (m, 4H), 6.7-6.8 (m, 3H), 6.27 (s, 1H), 3.63 (s, 3H), 2.18 (s, 3H); MS (EI) m/z: 401 and 403 (M⁺ and M⁺+2).

### Example 338

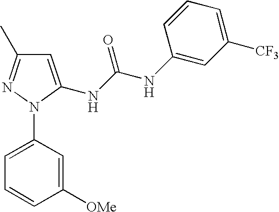

Using the same procedure as for Example 308, Example A5 (50 mg, 0.21 mmol) and 3-(trifluoromethyl)phenyl isocyanate (39 mg, 0.21 mmol) were combined to afford 1-(1-(3-methoxyphenyl)-3-methyl-1H-pyrazol-5-yl)-3-(3-(trifluoromethyl)phenyl)urea (32 mg, 39% yield). ¹H NMR (DMSO-d₆): δ 8.46 (bs, 1H), 7.53 (bs, 1H), 7.49 (s, 1H), 7.2-7.4 (m, 3H), 7.13 (t, J=8.0 Hz), 6.7-6.8 (m, 3H), 6.29 (s, 1H), 3.60 (s, 3H), 2.15 (s, 3H); MS (EI) m/z: 357 (M+H⁺).

### Example 339

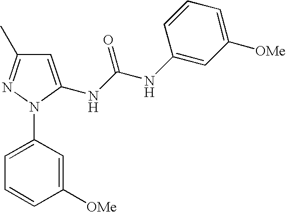

Using the same procedure as for Example 308, Example A5 (50 mg, 0.21 mmol) and 3-methoxyphenyl isocyanate (30 mg, 0.21 mmol) were combined to afford 1-(3-methoxyphenyl)-3-(1-(3-methoxyphenyl)-3-methyl-1H-pyrazol-5-yl)urea (6 mg, 8% yield). ¹H NMR (CDCl₃): δ 7.2-7.4 (m, 1H), 7.17(t, J=8.4 Hz, 1H), 6.99 (t, J=2.0 Hz, 1H), 6.9-7.0 (m, 2H), 6.86 (m, 1H), 6.76 (dd, J=1.2, and 8.0 Hz, 1H), 6.65 (dd, J=2.4, and 8.4 Hz, 1H), 6.34 (s, 1H), 3.78 (s, 3H), 3.75 (s, 3H), 2.28 (s, 3H); MS (EI) m/z: 353 (M+H⁺).

### Example 340

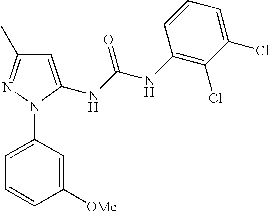

Using the same procedure as for Example 308, Example A5 (50 mg, 0.21 mmol) and 2,3-dichlorophenyl isocyanate (39 mg, 0.21 mmol) were combined to afford 1-(2,3-dichlorophenyl)-3-(1-(3-methoxyphenyl)-3-methyl-1H-pyrazol-5-yl)urea (23 mg, 28% yield). ¹H NMR (CDCl₃): δ 8.08 (m, 1H), 7.60 (s, 1H), 7.32(t, J=8.4 Hz, 1H), 7.19 (d, J=1.2 Hz, 1H), 7.18 (s, 1H), 7.01 (m, 2H), 6.97 (bs, 1H), 6.89 (dd, J=2.1, and 8.3 Hz, 1H), 6.35 (s, 1H), 3.79 (s, 3H), 2.35 (s, 3H); MS (EI) m/z: 391 (M+H⁺).

### Example A6

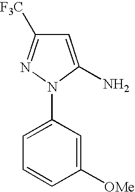

To a suspension of NaH (60% 6.0 g, 0.15 mol) in THF (100 ml) was added dropwise trifluoro-acetic acid ethyl ester (14.2 g, 0.1 mol) and anhydrous acetonitrile (50 g, 0.12 mol) in THF (100 ml) at 80° C. The resulting mixture was refluxed overnight, and then cooled to RT. After removal of the volatiles in vacuo, the residue was diluted in EtOAc and aqueous 10% HCL. The organic layer was washed with water and brine, dried (MgSO₄), filtered and concentrated to yield 15 g of the crude product, which was used for the next step reaction without further purification.

To a mixture of (3-methoxyphenyl)-hydrazine (690 mg, 5.0 mmol) and commercially available 4,4,4-trifluoro-3-oxo-butyronitrile (822 mg, 6.0 mmol) in ethanol (50 mL) was added conc. HCl (5 mL). The resulting mixture was heated to reflux for 3 h. After removal of the solvent, the residue was washed with Et₂O to afford 0.95 g of the crude 3-(trifluoromethyl)-1-(3-methoxyphenyl)-1H-pyrazol-5-amine, which was used to the next reaction without further purification. MS (ESI) m/z: 258 (M+H⁺).

### Example 341

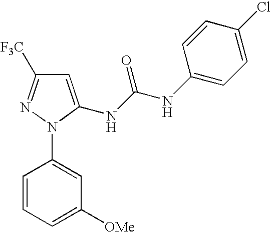

To a solution of Example A6 (100 mg, 0.39 mmol) and Et₃N (80 mg, 0.8 mmol) in THF (30 mL) was added 1-chloro-4-isocyanato-benzene (153 mg, 1.0 mmol) at 0° C. in ice-water bath. The resulting mixture was stirred at 0° C. for 30 min and then warmed to RT for 3 h. The reaction mixture was quenched with 1.0 N HCl and extracted with CH₂Cl₂ (3×100 mL). The combined organic extracts were washed with brine, dried (Na₂SO₄), filtered and concentrated to the crude product, which was purified by preparative HPLC to afford 85 mg of 1-(4-chlorophenyl)-3-(3-(trifluoromethyl)-1-(3-methoxyphenyl)-1H-pyrazol-5-yl)urea. ¹H NMR (300 MHz, DMSO-d6): δ 9.25 (s, 1H), 8.72 (s, 1H), 7.50 (t, J=6.3 Hz, 1H), 7.42 (d, J=6.6 Hz, 2H), 7.31 (d, J=6.6 Hz, 2H), 7.15-7.12 (m, 3H), 6.87 (s, 1H), 3.81 (s, 3H). MS (ESI) m/z: 411 (M+H⁺)

### Example 342

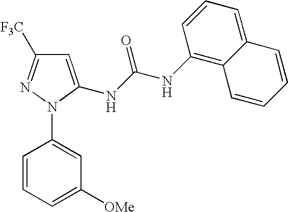

Using the same procedure as for Example 308, Example A6 (100 mg, 0.39 mmol) and 1-Isocyanato-naphthalene (169 mg, 1.0 mmol) were combined to afford 70 mg of 1-(3-(trifluoromethyl)-1-(3-methoxyphenyl)-1H-pyrazol-5-yl)-3-(naphthalen-1-yl)urea. ¹H NMR (300 MHz, DMSO-d6): δ 9.16 (s, 1H), 9.08 (s, 1H), 7.95-7.85 (m, 3H), 7.66 (d, J=6.0 Hz, 1H), 7.55-7.41 (m, 4H), 7.22-7.12 (m, 3H), 6.88 (s, 1H), 3.83 (s, 3H). MS (ESI) m/z: 427 (M+H⁺)

### Example A7

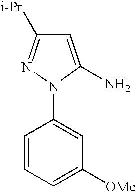

To a suspension of NaH (60%, 6.0 g, 0.15 mol) in THF (100 mL) was added dropwise isobutyric acid ethyl ester (11.6 g, 0.1 mol) and anhydrous acetonitrile (50 g, 0.12 mol) in THF (100 mL) at 80° C. The resulting mixture was refluxed overnight, then cooled to RT. After removal of the volatiles in vacuo, the residue was diluted in EtOAc and aqueous 10% HCL. The combined organic extracts were dried (Na₂SO₄), filtered, concentrated to yield 4-methyl-3-oxopentanenitrile (8.5 g), which was used for the next step reaction without further purification.

To a mixture of (3-methoxy-phenyl)-hydrazine (690 mg, 5.0 mmol) and 4-methyl-3-oxo-pentanenitrile (660 mg, 6.0 mmol) in ethanol (50 mL) was added conc. HCl (5 mL). The resulting mixture was heated to reflux for 3 h. After removal of the solvent, the residue was washed with Et₂O to afford 0.95 g of the crude 3-isopropyl-1-(3-methoxyphenyl)-1H-pyrazol-5-amine, which was used to the next reaction without further purification MS (ESI) m/z: 232 (M+H⁺).

### Example 343

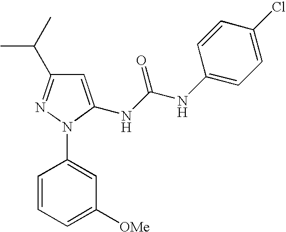

To a solution of Example A7 (100 mg, 0.43 mmol) and Et₃N (80 mg, 0.8 mmol) in THF (30 mL) was added 1-chloro-4-isocyanato-benzene (153 mg, 1.0 mmol) at 0° C. in an ice-water bath. The resulting mixture was stirred at 0° C. for 30 min and then warmed to RT for 3 h. The reaction mixture was quenched with 1.0 N HCl and extracted with CH₂Cl₂ (3×100 mL). The combined organic extracts were washed with brine, dried ((Na₂SO₄), filtered and concentrated to the crude product, which was purified by preparative HPLC to afford 85 mg of 1-(4-chlorophenyl)-3-(3-isopropyl-1-(3-methoxyphenyl)-1H-pyrazol-5-yl)urea. ¹H NMR (300 MHz, DMSO-d6): [symbol] 9.08 (s, 1H), 9.04 (s, 1H), 7.45 (d, J=6.0 Hz, 2H), 7.41 (t, J=6.6 Hz, 1H), 7.30 (d, J=6.6 Hz, 2H), 7.05-6.98 (m, 3H), 6.36 (s, 1H), 3.78 (s, 3H). 1.12 (d, J=5.1 Hz, 6H), MS (ESI) m/z: 385 (M+H⁺)

### Example 344

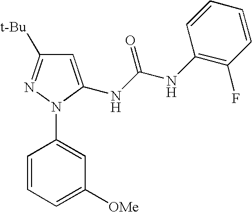

To a solution of Example A3 (123 mg, 0.5 mmol) and triethylamine (101 mg, 1.0 mmol) in anhydrous THF (5 mL) was added 1-fluoro-2-isocyanato-benzene (69 mg, 0.5 mmol) at 0° C. This resulted mixture was stirred at RT for 3 h, and extracted with EtOAc. The combined organic extracts were washed with brine, dried (Na₂SO₄), filtered, concentrated and purified by preparative TLC to afford 1-[3-t-butyl-1-(3-methoxy-phenyl)-1H-pyrazol-5-yl]-3-(2-fluorophenyl)urea. ¹H-NMR (300 MHz, DMSO-d₆): δ 8.92 (s, 1H), 8.80 (s, 1H), 8.06 (t, J=7.5 Hz, 1H), 7.39 (t, J=7.5 Hz, 1H), 7.17-6.96 (m, 6H), 6.35 (s, 1H), 3.75 (s, 3H), 1.22 (s, 9H); MS (ESI) m/z: 383(M+H⁺).

### Example 345

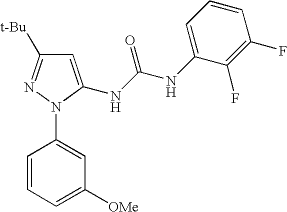

Using the same procedure as for Example 305, Example A3 (123 mg, 0.5 mmol) and 2,3-difluoro-phenylamine (65 mg, 0.5 mmol) were combined to afford 1-[3-t-butyl-1-(3-methoxy-phenyl)-1H-pyrazol-5-yl]-3-(2,3-difluorophenyl)urea. ¹H-NMR (300 MHz, DMSO-d₆): δ 9.11 (s, 1H), 8.84 (s, 1H), 7.87 (t, J=7.8 Hz, 1H), 7.40 (t, J=7.8 Hz, 1H), 7.09-6.94 (m, 5H), 6.36 (s, 1H), 3.76 (s, 3H), 1.23 (s, 9H); MS (ESI) m/z: 401(M+H⁺).

### Example A8

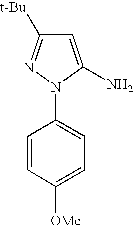

A mixture of (4-methoxy-phenyl)-hydrazine (17.4 g, 0.1 mol) and 4,4-dimethyl-3-oxo-pentanenitrile (13.8 g, 0.11 mol) in ethanol (500 mL) and conc. HCl (50 mL) was heated to reflux overnight. After removal of the solvent, the residue was purified by column chromatography to give 3-t-butyl-1-(4-methoxyphenyl)-1H-pyrazol-5-amine (20 g, 82% yield). ¹H-NMR (300 MHz, DMSO-d₆): δ 7.38 (d, J=9.0 Hz, 2H), 6.97 (d, J=9.0 Hz, 2H), 5.32 (s, 1H), 4.99 (br s, 2H), 3.75 (s, 3H), 1.17 (s, 9H); MS (ESI) m/z: 246 (M+H⁺).

### Example 346

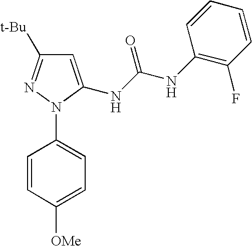

Using the same procedure as for Example 308, Example A8 (123 mg, 0.5 mmol) and 1-fluoro-2-isocyanato-benzene (69 mg, 0.5 mmol) were combined to afford 1-[3-t-butyl-1-(4-methoxyphenyl)-1H-pyrazol-5-yl]-3-(2-fluorophenyl)urea. ¹H-NMR (300 MHz, DMSO-d₆): δ 9.01 (s, 1H), 8.89 (s, 1H), 8.09 (t, J=7.8 Hz, 1H), 7.36 (d, J=8.7 Hz, 2H), 7.09-7.21 (m, 2H), 7.05 (d, J=8.7 Hz, 2H), 6.97 (t, J=8.7 Hz, 1H), 6.32 (s, 1H), 3.79 (s, 3H), 1.23 (s, 9H); MS (ESI) m/z: 383 (M+H⁺).

### Example 347

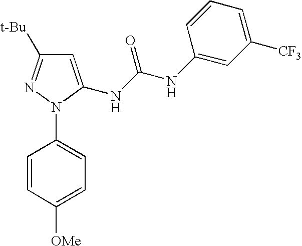

Using the same procedure as for Example 308, Example A8 (123 mg, 0.5 mmol) and 1-isocyanato-3-trifluoromethyl-benzene (93 mg, 0.5 mmol) were combined to afford 1-[3-t-butyl-1-(4-methoxyphenyl)-1H-pyrazol-5-yl]-3-(3-trifluoromethylphenyl)urea (65 mg, 30% yield). ¹H-NMR (300 MHz, DMSO-d₆): δ 9.38 (s, 1H), 8.40 (s,1H), 7.94 (br s, 1H), 7.45 (d, J=4.8 Hz, 2H), 7.38 (d, J=9.0 Hz, 2H), 7.27 (m, 1H), 7.03 (d, J=9.0 Hz, 2H), 6.32 (s, 1H), 3.78 (s, 3H), 1.24 (s, 9H); MS (ESI) m/z: 433 (M+H⁺).

### Example 348

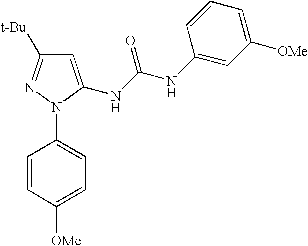

Using the same procedure as for Example 308, Example A8 (123 mg, 0.5 mmol) and 1-Isocyanato-3-methoxy-benzene (93 mg, 0.5 mmol) were combined to afford 1-[3-t-butyl-1-(4-methoxyphenyl)-1H-pyrazol-5-yl]-3-(3-methoxy-phenyl)urea (65 mg, 33% yield). ¹H-NMR (300 MHz, DMSO-d₆): δ 9.98 (s, 1H), 8.25 (s, 1H), 7.37 (d, J=8.7 Hz, 2H), 7.13-7.03 (m, 4H), 6.82 (d, J=6.9 Hz, 1H), 6.52 (d, J=6.9 Hz, 1H), 6.31 (s, 1H), 3.78 (s, 3H), 1.23 (s, 9H); MS (ESI) m/z: 395 (M+H⁺).

### Example 349

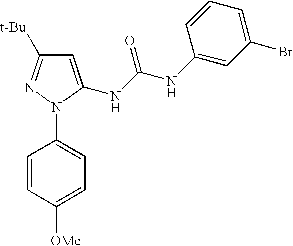

Using the same procedure as for Example 308, Example A8 (123 mg, 0.5 mmol) and 1-bromo-3-isocyanato-benzene (98 mg, 0.5 mmol) were combined to afford 1-(3-bromophenyl)-3-[3-t-butyl-1-(4-methoxyphenyl)-1H-pyrazol-5-yl]urea (65 mg, 29% yield). ¹H-NMR (300 MHz, DMSO-d₆): δ 9.18 (s, 1H), 8.34 (s, 1H), 7.80 (br s, 1H), 7.37 (d, J=9.0 Hz, 2H), 7.18 (d, J=5.1 Hz, 2H), 7.12 (m, 1H), 7.03 (d, J=9.0 Hz, 2H), 6.31 (s, 1H), 3.78 (s, 3H), 1.24 (s, 9H); MS (ESI) m/z: 443 (M+H⁺).

### Example 350

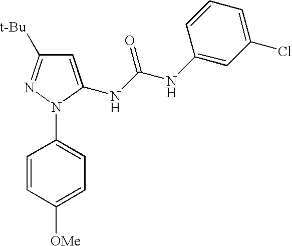

Using the same procedure as for Example 308, Example A8 (123 mg, 0.5 mmol) and 1-chloro-3-isocyanato-benzene (76 mg, 0.5 mmol) were combined to afford 1-[3-t-butyl-1-(4-methoxyphenyl)-1H-pyrazol-5-yl]-3-(3-chlorophenyl)urea (65 mg, 33% yield). ¹H-NMR (300 MHz, DMSO-d₆): δ 9.17 (s, 1H), 8.34 (s, 1H), 7.65 (t, J=2.1 Hz, 1H), 7.37 (d, J=9.0 Hz, 2H), 7.22 (m, 1H), 7.15 (m, 1H), 6.31 (s, 1H), 3.78 (s, 3H), 1.24 (s, 9H); MS (ESI) m/z: 399 (M+H⁺).

### Example A9

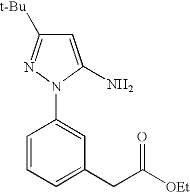

A mixture of 1-(3-nitrophenyl)ethanone (82.5 g, 0.5 mol), toluene-4-sulfonic acid (3 g) and sulfur (32 g, 1.0 mol) in morpholine (100 mL) was heated to reflux for 3 h. After removal of the solvent, the residue was dissolved in dioxane (100 mL). The mixture was added concentrated HCl (100 mL) and then heated to reflux for 5 h. After removal of the solvent, the residue was extracted with EtOAc (3×150 mL). The combined organic extracts were washed with brine, dried (Na₂SO₄), filtered, and concentrated. The residue was dissolved in ethanol (250 mL) and SOCl₂ (50 mL) and heated to reflux for 2 h. After removal of the solvent, the residue was extracted with EtOAc (3×150 mL). The combined organic extracts were washed with brine, dried (Na₂SO₄), filtered, concentrated and purified via column chromatography to afford ethyl (3-nitrophenyl)acetate (40 g). ¹H-NMR (300 MHz, DMSO-d₆): δ 8.17 (s, 1H), 8.11 (d, J=7.2 Hz, 1H), 7.72 (d, J=7.2 Hz, 1H), 7.61 (t, J=7.8 Hz, 1H), 4.08 (q, J=7.2 Hz, 2H), 3.87 (s, 2H), 1.17 (t, J=7.2 Hz, 3H)

A mixture of (3-nitrophenyl)acetic acid ethyl ester (21 g, 0.1 mol) and Pd/C (2 g) in methanol (300 mL) was stirred at RT under 40 psi of H₂ for 2 h. The reaction mixture was filtered and the filtrate was concentrated to afford ethyl (3-aminophenyl)acetate (17 g). MS (ESI) m/z: 180 (M+H⁺).

To a suspension of (3-aminophenyl)acetic acid (17 g, 94 mmol) in concentrated HCl (50 mL) was added dropwise a solution of sodium nitrite (6.8 g, 0.1 mol) in water at 0° C. The mixture was stirred for 1 h, after which a solution of SnCl₂ (45 g, 0.2 mol) in concentrated HCl was added dropwise at such a rate that the reaction mixture never rose above 5° C. The resulted mixture was stirred for 2 h. The precipitate was collected by suction, washed with ethyl ether to afford ethyl (3-hydrazinophenyl)acetate (15 g). MS (ESI) m/z: 195 (M+H⁺)

A solution of ethyl (3-hydrazinophenyl)acetate (15 g, 65 mmol) and 4,4-dimethyl-3-oxopentanenitrile (12.5 g, 0.1 mol) in EtOH (100 mL) containing concentrated HCl (25 mL) was heated to reflux overnight. After removal of the solvent, the residue was washed with Et₂O to afford ethyl 2-(3-(5-amino-3-t-butyl-1H-pyrazol-1-yl)phenyl)acetate (18 g). MS (ESI) m/z: 302 (M+H⁺).

### Example A10

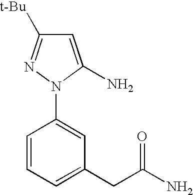

To a solution of Example A8 (6.0 g, 20 mmol) and formamide (1.8 g, 40 mmol) in DMF (20 mL) was added NaOMe (2.1 g, 40 mmol) at RT. The mixture was heated to reflux for 1 h, concentrated and the residue was purified via column chromatography to afford 2-[3-(5-amino-3-t-butyl-1H-pyrazol-1-yl)phenyl]acetamide (2.0 g, 40% yield). ¹H NMR (300 MHz, DMSO-d₆): δ 7.44-7.31 (m, 4H), 7.11 (m, 1H), 6.87 (br s, 1H), 5.33 (s, 1H), 5.12 (s, 2H), 3.38 (s, 2H), 1.17 (s, 9H); MS (ESI) m/z: 273 (M+H⁺).

### Example 351

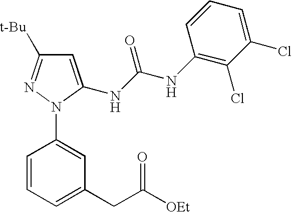

Using the same procedure as for Example 302, Example A9 (2.0 g, 6.6 mmol) and 1,2-dichloro-3-isocyanato-benzene (1.1 g, 7.5 mmol) were combined to afford 2.2 g of ethyl 2-(3-(3-t-butyl-5-(3-(2,3-dichlorophenyl)ureido)-1H-pyrazol-1-yl)phenyl)acetate. ¹H NMR (300 MHz, DMSO-d₆): 9.22 (s, 1H), 8.75 (s, 1H), 8.05 (m, 1H), 7.46-7.21 (m, 6H), 6.35 (s, 1H), 4.04 (q, J=7.2 Hz, 2H), 3.72 (s, 2H), 1.24 (s, 9H), 1.16 (t, J=7.2 Hz, 3H); MS (ESI) m/z: 489 (M+H⁺).

### Example 352

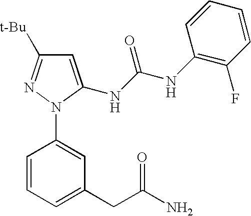

Using the same procedure as for Example 302, Example A10 (136 mg, 0.5 mmol) and 1-fluoro-2-isocyanatobenzene (68 mg, 0.5 mmol) were combined to afford 55 mg of 1-{1-[3-(2-amino-2-oxoethyl)phenyl]-3-t-butyl-1H-pyrazol-5-yl}-3-(2-fluorophenyl)urea (55 mg, 27% yield). ¹H NMR (300 MHz, DMSO-d₆): δ 8.90 (br s, 1H), 8.85 (br s, 1H), 8.05 (br s, 1H), 7.50-7.20 (m, 5H), 7.20-7.00 (m, 2H), 7.00-6.80 (m, 2H), 6.34 (s, 1H), 3.41 (s, 2H), 1.22 (s, 9H).

### Example 353

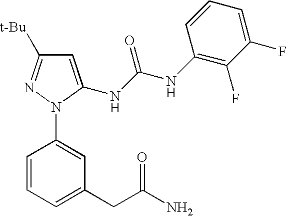

Using the same procedure as for Example 302, Example A10 (136 mg, 0.5 mmol) and 2,3-difluoroaniline (65 mg, 0.5 mmol) were combined to afford 1-{1-[3-(2-amino-2-oxoethyl)phenyl]-3-t-butyl-1H-pyrazol-5-yl}-3-(2,3-difluorophenyl)urea (60 mg, 28% yield). ¹H NMR (300 MHz, CD₃OD-d₄): δ 7.86 (m, 1H), 7.55-7.37 (m, 4H), 7.08 (m, 1H), 6.89 (m, 1H), 6.46 (s, 1H), 3.63 (s, 2H), 1.32 (s, 9H); MS (ESI) m/z: 428 (M+H⁺).

### Example A11

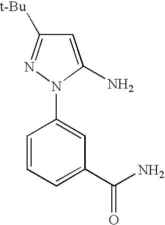

To a solution of m-aminobenzoic acid (200.0 g, 1.46 mmol) in concentrated HCl (200 mL) was added an aqueous solution (250 mL) of NaNO₂ (102 g, 1.46 mmol) at 0° C. and the reaction mixture was stirred for 1 h. A solution of SnCl₂.2H₂O (662 g, 2.92 mmol) in concentrated HCl (2000 mL) was then added at 0° C. The reaction solution was stirred for an additional 2 h at RT. The precipitate was filtered and washed with ethanol and ether to give 3-hydrazino-benzoic acid hydrochloride as a white solid, which was used for the next reaction without further purification. ¹H NMR (DMSO-d₆): 10.85 (s, 3H), 8.46 (s, 1. H), 7.53 (s, 1H), 7.48 (d, J=7.6 Hz, 1H), 7.37 (m, J=7.6 Hz, 1H), 7.21 (d, J=7.6 Hz, 1H).

A mixture of 3-hydrazino-benzoic acid hydrochloride (200 g, 1.06 mol) and 4,4-dimethyl-3-oxo-pentanenitrile (146 g, 1.167 mol) in ethanol (2 L) was heated to reflux overnight. The reaction solution was evaporated under reduced pressure. The residue was purified by column chromatography to give 3-(5-amino-3-t-butyl-pyrazol-1-yl)-benzoic acid ethyl ester (116 g, 40%) as a white solid together with 3-(5-amino-3-t-butyl-pyrazol-1-yl)-benzoic acid (93 g, 36%). 3-(5-amino-3-t-butyl-pyrazol-1-yl)-benzoic acid and ethyl ester: ¹H NMR (DMSO-d₆): 8.09 (s, 1H), 8.05 (brd, J=8.0 Hz, 1H), 7.87 (br d, J=8.0 Hz, 1H), 7.71 (t, J=8.0 Hz, 1H), 5.64 (s, 1H), 4.35 (q, J=7.2 Hz, 2H), 1.34 (t, J=7.2 Hz, 3H), 1.28 (s, 9H).

### Example A12

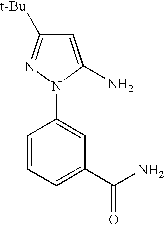

To a solution of T (14.4 g, 50 mmol) and formamide (4.5 g, 0.1 mol) in DMF (50 mL) was added NaOMe (5.4 g 0.1 mol) at RT. The mixture was stirred at 100° C. for 1 h, concentrated and the residue purified by column chromatography to afford 3-(5-amino-3-t-butyl-1H-pyrazol-1-yl)benzamide (6 g, 48% yield).

### Example A13

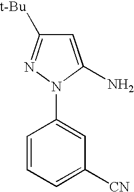

A solution of Example A12 (5.2 g, 20 mmol) in SOCl₂ (50 mL) was heated to reflux for 6 h. After removal of the solvent, the residue was dissolved in EtOAc (100 mL). The organic layer was washed with saturated NaHCO₃ and brine, dried (Na₂SO₄), filtered, and purified by column chromatography to afford 3-(5-amino-3-t-butyl-1H-pyrazol-1-yl)benzonitrile (3.5 g, 73% yield).

### Example 354

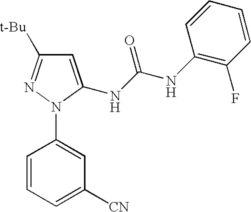

Using the same procedure as for Example 304, Example A13 (120 mg, 0.5 mmol) and 1-fluoro-2-isocyanate-benzene (68 mg, 0.5 mmol) were combined to afford 1-[3-t-butyl-1-(3-cyanophenyl)-1H-pyrazol-5-yl]-3-(2-fluorophenyl)urea (55 mg, 29% yield). ¹H NMR (300 MHz, DMSO-d₆): δ 8.90 (br s, 2H), 8.04-7.99 (m, 2H), 7.85 (t, J=8.1 Hz, 2H), 7.70 (t, J=8.1 Hz, 2H), 7.20 (m, 1H), 7.09 (m, 1H), 6.99 (m, 1H), 6.40 (s, 1H), 1.25 (s, 9H); MS (ESI) m/z: 378 (M+H⁺).

### Example 355

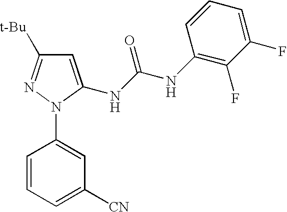

Using the same procedure as for Example 305, Example A13 (120 mg, 0.5 mmol) and 2,3-difluoro-phenylamine (129 mg, 1.0 mmol) were combined to afford 1-[3-t-butyl-1-(3-cyan-phenyl)-1H-pyrazol-5-yl]-3-(2,3-difluorophenyl)urea (55 mg, 28% yield). ¹H NMR (300 MHz, DMSO-d₆): δ 9.07 (br s, 1H), 8.92 (s, 1H), 8.00 (s, 1H), 7.88-7.81 (m, 3H), 7.73 (t, J=7.8 Hz, 1H), 7.12-6.97 (m, 2H), 6.40 (s, 1H), 1.25 (s, 9H); MS (ESI) m/z: 396 (M+H⁺).

### Example 356

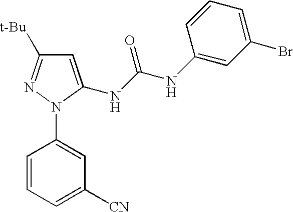

To a stirring suspension of Example A13 (0.0500 g, 0.208 mmol, 1.00 eq) in dry THF (2.0 ml) was added pyridine (0.168 ml, 2.08 mmol, 10.00 eq). The resulting slurry was stirred at RT for 1 h, treated with 3-bromophenyl isocyanate (0.0520 ml, 0.416 mmol, 2.00 eq) and stirred overnight at RT. The reaction was diluted with EtOAc and 1M HCl (10 ml) and the layers separated. The aqueous was extracted with EtOAc (2×), and the combined organic extracts were washed with H₂O (1×), satd. NaHCO3 (1×) and brine (2×), dried (MgSO4), filtered, concentrated, and purified via column chromatography to yield 1-(3-t-butyl-1-(3-cyanophenyl)-1H-pyrazol-5-yl)-3-(3-bromophenyl)urea as an oil (38.4 mg, 42% yield). ¹H NMR (CDCl₃): δ 7.77-7.70 (m, 3H), 7.52 (s, 1H), 7.46-7.44 (m, 2H), 7.35 (s, 1H), 7.16-7.13 (m, 1H), 7.06-7.04 (m, 2H), 6.35 (s, 1H), 1.29 (s, 9H); MS (ESI) m/z: 438.0 (M+H⁺), 440.0 (M+2+H⁺).

### Example 357

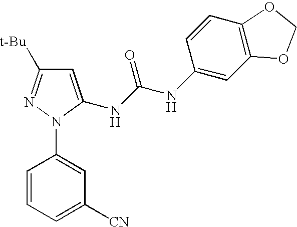

Using the same procedure as for Example 356, Example A13 (0.500 g, 1.81 mmol, 1.00 eq) and 3,4-(methylenedioxy)phenyl isocyanate (0.59 g, 3.62 mmol) were combined to afford 1-(benzo[d][1,3]dioxol-5-yl)-3-(3-t-butyl-1-(3-cyanophenyl)-1H-pyrazol-5-yl)urea as an off-white solid (107.4 mg, 15% yield). ¹H NMR (DMSO-d₆): δ 8.92 (s, 1H), 8.47 (s, 1H), 8.02-8.01 (m, 1H), 7.91-7.89 (m, 1H), 7.86-7.84 (m, 1H), 7.75-7.71 (m, 1H), 7.12-7.11 (m, 1H), 6.82-6.79 (m, 1H), 6.73-6.70 (m, 1H), 6.39 (s, 1H), 5.96 (s, 2H), 1.28 (s, 9H); MS (ESI) m/z: 404.2 (M+H⁺).

### Example 358

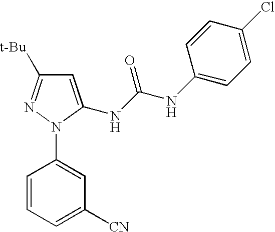

Using the same procedure as for Example 356, Example A13 (0.500 g, 1.81 mmol, 1.00 eq) and 4-chlorophenyl isocyanate (0.555 g, 3.61 mmol) were combined to afford 1-(3-t-butyl-1-(3-cyanophenyl)-1H-pyrazol-5-yl)-3-(4-chlorophenyl)urea (264 mg, 37% yield). ¹H NMR (CDCl₃): δ 7.87 (s, 1H), 7.81-7.79 (m, 1H), 7.58-7.53 (m, 3H), 7.26 (brs, 3H), 6.48 (brs, 1H), 1.37 (s, 9H); MS (ESI) m/z: 394.2 (M+H⁺).

### Example 359

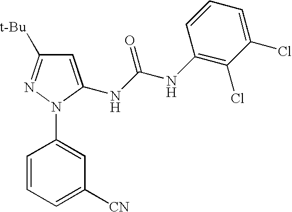

Using the same procedure as for Example 356, Example A13 (0.0500 g, 0.208 mmol) and 2,3-dichlorophenylisocyanate (0.0549 mL, 0.416 mmol) were combined to afford 1-(3-t-butyl)-1-(3-cyanophenyl)-1H-pyrazol-5-yl)-3-(2,3-dichlorophenyl)urea as a white solid (16.9 mg, 19% yield). ¹H NMR (CDCl₃): δ 8.12-8.09 (m, 1H), 7.95 (s, 1H), 7.85-7.83 (m, 1H), 7.64-7.54 (m, 3H), 7.25-7.19 (m, 2H), 6.52 (s, 1H), 1.40 (s, 9H); MS (ESI) m/z: 428.0 (M+H⁺), 430.0 (M+2+H⁺).

### Example 360

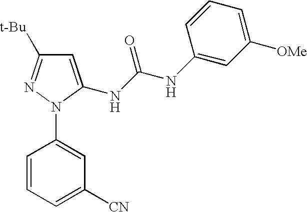

Using the same procedure as for Example 356, Example A13 (0.0500 g, 0.208 mmol) and 3-methoxyphenyl isocyanate (0.0545 mL, 0.416 mmol) were combined to afford 1-(3-t-butyl)-1-(3-cyanophenyl)-1H-pyrazol-5-yl)-3-(3-methoxyphenyl)urea as an oil (15 mg, 19% yield). ¹H NMR (CDCl₃): δ 7.78-7.75 (m, 2H), 7.51-7.44 (m, 2H), 7.33 (s, 1H), 7.24 (s, 1H), 7.18-7.14 (m, 1H), 6.93-6.91 (m, 1H), 6.72-6.70 (m, 1H), 6.65-6.62 (m, 1H), 6.38 (s, 1H), 3.74 (s, 3H), 1.32 (s, 9H); MS (ESI) m/z: 390.2 (M+H⁺).

### Example 361

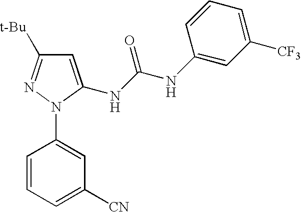

Using the same procedure as for Example 356, Example A13 (0.0500 g, 0.208 mmol) and α,α,α-trifluoro-m-tolyl isocyanate (0.0573 mL, 0.416 mmol) were combined to afford 1-(3-t-butyl)-1-(3-cyanophenyl)-1H-pyrazol-5-yl)-3-(3-(trifluoromethyl)phenyl)urea as an oil (36.7 mg, 41% yield). ¹H NMR (DMSO-d₆): δ 9.40 (s, 1H), 8.64 (s, 1H), 8.05-8.04 (m, 1H), 7.97 (s, 1H), 7.94-7.91 (m, 1H), 7.86-7.84 (m, 1H), 7.75-7.71 (m, 1H), 7.55-7.48 (m, 2H), 7.32-7.31 (m, 1H), 6.44 (s, 1H), 1.30 (s, 9H); MS (ESI) m/z: 428.3 (M+H⁺).

### Example A14

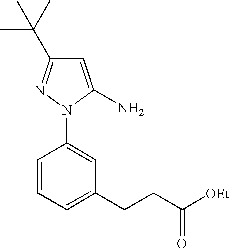

To a solution of 3-nitro-benzaldehyde (15.1 g, 0.1 mol) in CH₂Cl₂ (200 mL) was added dropwise (triphenyl-15-phosphanylidene)-acetic acid ethyl ester (34.8 g, 0.1 mol) in CH₂Cl₂ (100 mL) at 0° C. After the addition was complete, the resulting mixture was stirred for 1 h. After removal the solvent under reduced pressure, the residue was purified by column chromatography to afford 3-(3-nitro-phenyl)-acrylic acid ethyl ester (16.5 g, 74.6%). ¹H-NMR (400 MHz, CDCl₃): 8.42 (s, 1H), 8.23 (dd, J=0.8 8.0 Hz, 1H), 7.82 (d, J=7.6 Hz, 1H), 7.72 (d, J=16.0 Hz, 1H), 7.58 (t, J=8.0 Hz, 1H), 6.56 (d, J=16.0 Hz, 1H), 4.29 (q, J=7.2 Hz, 2H), 1.36 (t, J=6.8 Hz, 3H).

A mixture of 3-(3-nitro-phenyl)-acrylic acid ethyl ester (16.5 g, 74.6 mmol) and Pd/C (1.65 g) in methanol (200 mL) was stirred under 40 psi of H₂ at RT for 2 h, then filtered through celite. After removal the solvent, 14 g of 3-(3-amino-phenyl)-propionic acid ethyl ester was obtained. ¹H-NMR (400 MHz, CDCl₃): 7.11 (t, J=5.6 Hz, 1H), 6.67 (d, J=7.2 Hz, 1H), 6.63-6.61 (m, 2H), 4.13 (q, J=7.2 Hz, 2H), 2.87 (t, J=8.0 Hz, 2H), 2.59 (t, J=7.6 Hz, 2H), 1.34 (t, J=6.8 Hz, 3H); MS (ESI): m/z: 194 (M+H⁺).

To a solution of 3-(3-amino-phenyl)-propionic acid ethyl ester (14 g, 72.5 mmol) in concentrated HCl (200 mL) was added aqueous (10 mL) of NaNO₂ (5 g, 72.5 mmol) at 0° C. and the resulting mixture was stirred for 1 h. A solution of SnCl₂.2H₂O (33 g, 145 mmol) in concentrated HCl (150 mL) was then added at 0° C. The reaction solution was stirred for an additional 2 h at RT. The precipitate was filtered and washed with ethanol and ether to yield 3-(3-hydrazino-phenyl)-propionic acid ethyl ester as a white solid, which was used for the next reaction without further purification. MS (ESI): m/z: 209 (M+H⁺)

A mixture of 3-(3-hydrazino-phenyl)-propionic acid ethyl ester (13 g, 53.3 mmol) and 4,4-dimethyl-3-oxo-pentanenitrile (6.9 g, 55 mol) in ethanol (150 mL) was heated to reflux overnight. The reaction solution was evaporated under vacuum. The residue was purified by column chromatography to yield ethyl 3-(3-(3-t-butyl-5-amino-1H-pyrazol-1-yl)phenyl)propanoate (14.3 g, 45.4 mmol) as a white solid. ¹H-NMR (300 MHz, DMSO-d₆); 7.50-7.42 (m, 4H), 5.63 (s, 1H), 5.14 (s, 2H), 4.04 (q, J=6.9 Hz, 2H), 2.92 (t, J=7.5 Hz, 2H), 2.66 (t, J=7.5 Hz, 2H), 1.27 (s, 9H), 1.16 (t, J=7.5 Hz, 3H) MS (ESI) m/z:316 (M+H⁺)

### Example 362

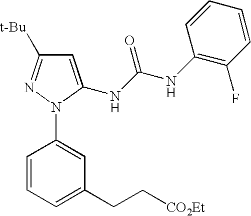

Using the same procedure as for Example 304, Example A14 (101 mg, 1.0 mmol) and 1-fluoro-2-isocyanato-benzene (137 mg, 1.0 mmol) were combined to afford 3-(3-{3-t-butyl-5-[3-(2-fluorophenyl)-ureido]-1H-pyrazol-1-yl}phenyl)propionic acid ethyl ester (240 mg, 53% yield), which was used with further purification.

### Example 363

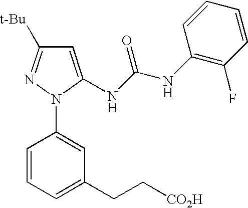

Using the same procedure as for Example 3067, Example 362 (100 mg, 0.221 mmol) was saponified to afford 3-(3-{3-t-butyl-5-[3-(2-fluorophenyl)ureido]-11H-pyrazol-1-yl}-phenyl)propionic acid (80 mg, 85% yield). ¹H NMR (300 MHz, DMSO-d₆): δ 8.90 (br s, 1H), 8.81 (s, 1H), 7.08 (t, J=7.5 Hz, 1H), 7.42 (t, J=7.5 Hz, 1H), 7.35 (s, 1H), 7.28 (t, J=6.9 Hz, 1H), 7.28 (m, 1H), 7.07 (t, J=7.5 Hz, 1H), 6.98 (m, 1H), 6.37 (s, 1H), 2.87 (t, J=7.5 Hz, 2H), 2.55 (t, J=7.5 Hz, 2H), 1.24 (s, 9H); MS (ESI) m/z: 425 (M+H⁺).

### Example 364

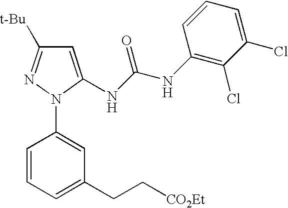

Using the same procedure as for Example 304, Example A14 (300 mg, 1.0 mmol) and 1,2-dichloro-3-isocyanato-benzene (187 mg, 1.0 mmol) were combined to afford 3-(3-{3-t-butyl-5-[3-(2,3-dichlorophenyl)ureido]-1H-pyrazol-1-yl}phenyl)propionic acid ethyl ester (210 mg, 42% yield), which was used without further purification ¹H NMR (DMSO-d₆): δ 9.20 (s, 1H), 8.76 (s, 1H), 8.05 (m, 1H), 7.47-7.26 (m, 6H), 6.38 (s, 1H), 4.04 (q, J=7.2 Hz, 2H), 2.93 (t, J=7.5 Hz, 2H), 2.65 (t, J=7.5 Hz, 2H), 1.28 (s, 9H), 1.15 (t, J=7.2 Hz, 3H); MS (ESI) m/z: 503 (M+H⁺).

### Example 365

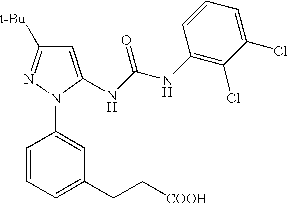

Using the same procedure as for Example 306, Example 365 (100 mg, 0.199 mmol) was saponified to afford 3-(3-{3-t-Butyl-5-[3-(2,3-dichloro-phenyl)ureido]-1H-pyrazol-1-yl}-phenyl)propionic acid (60 mg, 63% yield). ¹H NMR (DMSO-d₆): δ 9.23 (s, 1H), 8.77 (s, 1H), 8.03 (m, 1H), 7.44-7.21 (m, 6H), 6.36 (s, 1H), 2.88 (t, J=7.5 Hz, 2H), 2.58 (t, J=7.5 Hz, 2H), 1.26 (s, 9H); MS (ESI) m/z: 475 (M+H⁺).

### Example 366

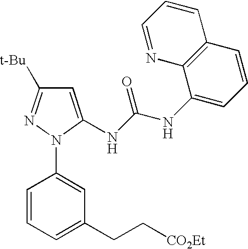

To a solution of Example A14 (150 mg, 0.48 mmol) and NaHCO₃ (200 mg, 2.4 mmol in THF (10 mL) was added a solution of triphosgene (50 mmg, 16 mmol) in THF (1 mL) at 0° C. The mixture was stirred at RT for 1 h, and was subsequently treated with a solution of quinolin-8-ylamine (72 mg, 0.50 mmol) in THF (2 mL). The resulted mixture was stirred for 3 h and concentrated. The residue was dissolved in CH₂Cl₂ (50 mL), and the organic layer was washed with brine, dried (Na₂SO₄), filtered, concentrated and purified by preparative HPLC to afford 3-(3-{3-t-butyl-5-[3-(quinolin-8-yl)ureido]-1H-pyrazol-1-yl}phenyl)-propionic acid ethyl ester (120 mg, 52% yield). ¹H NMR (DMSO-d₆): δ 9.91 (s, 1H), 9.54 (s, 1H), 8.84 (d, J=5.6 Hz, 1H), 8.49 (d, J=6.8 Hz, 1H), 8.35 (d, J=8.0 Hz, 1H), 7.58-7.60 (m, 1H), 7.51-7.53 (m, 2H), 7.36-7.41 (m, 3H), 7.24 (d, J=7.2 Hz, 1H), 6.40 (s, 1H), 3.96 (q, J=7.2 Hz, 1H), 2.88 (t, J=7.6 Hz, 2H), 2.60 (t, J=7.6 Hz, 2H), 1.28 (s, 9H), 1.07 (t, J=7.2 Hz, 3H); MS (ESI) m/z: 486 (M+H⁺).

### Example 367

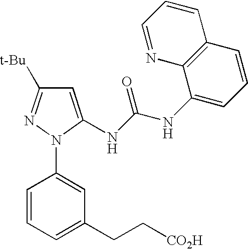

Using the same procedure as for Example 306, Example 366 (70 mg, 0.14 mmol) was saponified to afford 3-(3-{3-t-butyl-5-[3-(quinolin-8-yl)ureido]-1H-pyrazol-1-yl}phenyl)-propionic acid (50 mg, 78% yield). ¹H NMR (DMSO-d₆): δ 9.93 (s, 1H), 9.56 (s, 1H), 8.84 (d, J=4.0 Hz, 1H), 8.50 (d, J=6.8 Hz, 1H), 8.36 (d, J=6.8 Hz, 1H), 7.60 (m, 1H), 7.40-7.50 (m, 4H), 7.34 (d, J=8.4 Hz, 1H), 7.25 (d, J=7.6 Hz, 1H), 6.41 (s, 1H), 2.87 (t, J=7.6 Hz, 2H), 2.55 (t, J=7.6 Hz, 2H), 1.23 (s, 9H); MS (ESI) m/z: 458 (M+H⁺).

### Example A15

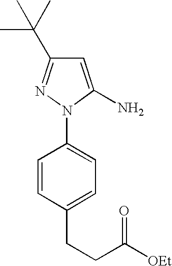

To a solution of 4-nitro-benzaldehyde (15.1 g, 0.1 mol) in CH₂Cl₂ (200 mL) was added dropwise (triphenyl-15-phosphanylidene)-acetic acid ethyl ester (34.8 g, 0.1 mol) in dichloromethane (100 mL) under 0° C. in ice-bath. After the addition was completed, the resulting mixture was stirred for 2 h. After removal the solvent under reduced pressure, the residue was purified by column chromatography to afford 3-(4-nitro-phenyl)-acrylic acid ethyl ester (16.5 g, 74.6%) ¹H-NMR (400 MHz, CDCl₃): 8.25 (d, J=8.8 Hz, 2H), 7.71 (d, J=16.0 Hz, 1H), 7.67 (d, J=8.8 Hz, 2H), 6.55 (d, J=16.0 Hz, 2H), 4.29 (q, J=7.2 Hz, 2H), 1.34 (t, J=7.2 Hz, 3H).

A mixture of 3-(4-nitro-phenyl)-acrylic acid ethyl ester (16.5 g, 74.6 mmol) and Pd/C (1.65 g) in methanol (200 mL) was stirred under 40 psi of H₂ at RT at 2 h before filtered over celite. After removal the solvent, 14 g of 3-(4-amino-phenyl)-propionic acid ethyl ester was obtained. ¹H-NMR (400 MHz, CDCl₃): 6.98 (d, J=8.0 Hz, 2H), 6.61 (d, J=8.4 Hz, 1H), 4.12 (q, J=7.2 Hz, 2H), 2.84 (t, J=8.0 Hz, 2H), 2.55 (t, J=7.6 Hz, 2H), 1.23 (t, J=7.2 Hz, 3H); MS (ESI): m/z: 194 (M+H⁺).

To a solution of 3-(4-amino-phenyl)-propionic acid ethyl ester (14 g, 72.5 mmol) in conc. HCl (200 mL) was added aqueous (10 mL) of NaNO₂ (5 g, 72.5 mmol) at 0° C. and the resulting mixture was stirred for 1 h. A solution of SnCl₂.2H₂O (33 g, 145 mmol) in conc. HCl (150 mL) was then added at 0° C. The reaction solution was stirred for an additional 2 h at RT. The precipitate was filtered and washed with ethanol and ether to yield 3-(4-hydrazino-phenyl)-propionic acid ethyl ester as a white solid, which was used for the next reaction without further purification; MS (ESI): m/z: 209 (M+H⁺).

A mixture of 3-(4-hydrazino-phenyl)-propionic acid ethyl ester (13 g, 53.3 mmol) and 4,4-dimethyl-3-oxo-pentanenitrile (6.9 g, 55 mol) in ethanol (150 mL) was heated to reflux overnight. The reaction solution was evaporated under vacuum. The residue was purified by column chromatography to yield ethyl 3-(4-(3-t-butyl-5-amino-1H-pyrazol-1-yl)phenyl)-propanoate (14.3 g, 45.4 mmol) as a white solid. ¹H-NMR (300 MHz, DMSO-d₆); 7.44 (d, J=8.4 Hz, 2H), 7.27 (d, J=8.7 Hz, 2H), 5.34 (s, 1H), 5.11 (s, 2H), 4.04 (q, J=7.2 Hz, 2H), 2.86 (t, J=7.5 Hz, 2H), 2.61 (t, J=7.5 Hz, 2H), 1.19 (s, 9H), 1.15 (t, J=7.2 Hz, 3H) MS (ESI) m/z: 316 (M+H⁺)

### Example 368

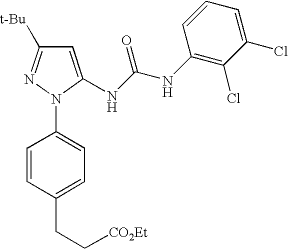

Using the same procedure as for Example 304, Example A15 (300 mg, 1.0 mmol) and 1,2-dichloro-3-isocyanato-benzene (187 mg, 1.0 mmol) were combined to afford 3-(4-{3-t-butyl-5-[3-(2,3-dichlorophenyl)ureido]-1H-pyrazol-1-yl}phenyl)propionic acid ethyl ester (250 mg, 50% yield), which was used without further purification. MS (ESI) m/z: 503 (M+H⁺).

### Example 369

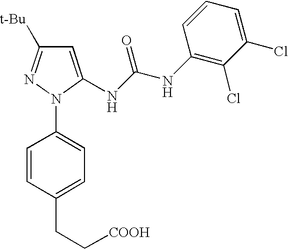

Using the same procedure as for Example 306, Example 368 (100 mg, 0.199 mmol) was saponified to afford 60 mg of 3-(3-{3-t-Butyl-5-[3-(2,3-dichloro-phenyl)-ureido]-pyrazol-1-yl}-phenyl)-propionic acid (60 mg, 64% yield). ¹H NMR (DMSO-d₆): δ 9.29 (s, 1H), 8.80 (s, 1H), 8.04 (m, 1H), 7.44-7.33 (m, 4H), 7.29-7.27 (m, 2H), 6.36 (s, 1H), 2.87 (t, J=7.5 Hz, 2H), 2.57 (t, J=7.5 Hz, 2H), 1.25 (s, 9H); MS (ESI) m/z: 475 (M+H⁺).

### Example A16

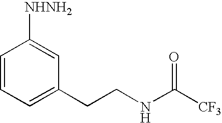

To a stirring solution of 3-nitrophenylacetic acid (10.4 g, 57.3 mmol) in MeOH (250 ml) at RT was added HCl gas until saturation was achieved. The resulting solution was stirred at 70° C. for 1 h. The reaction was cooled and concentrated under reduced pressure. The semisolid residue was dissolved in Et₂O, washed with H₂O (2×), sat'd. NaHCO₃ (2×), brine (1×) and dried (MgSO₄). Filtration and evaporation provided methyl 2-(3-nitrophenyl)acetate as a low-melting solid (10.7 g, 96% yield), which was used without further purification. ¹H NMR (CDCl₃): δ 8.14-8.04 (m, 2H), 7.64-7.58 (m, 1H), 7.47 (br t, J=8.10 Hz, 1H), 3.72 (s, 2H), 3.68 (s, 3H); MS (ESI) m/z: 196.0 (M+H⁺).

Methyl 2-(3-nitrophenyl)acetate (9.6 g, 49 mmol) was treated with conc. NH₄OH (24 ml, 172 mmol). The suspension was stirred briskly at RT until complete, then chilled thoroughly in an ice bath. The solids were collected by filtration, rinsed sparingly with ice water and dried to yield pure 2-(3-nitrophenyl)acetamide as an off-white solid (7.47 g, 84% yield)). ¹H NMR (DMSO-d₆): δ 8.18-8.02 (m, 2H), 7.75-7.70 (m, 1H), 7.61-7.57 (m, 3H), 7.00 (br s, 1H), 3.58 (s, 3H); MS (ESI) m/z: 181.0 (M+H⁺).

To a stirring solution of borane-THF (3.5 ml, 3.5 mmol, 1.0M) was added a solution of 2-(3-nitrophenyl)acetamide (0.25 g, 1.4 mmol) in THF (7.0 ml) at RT. The resulting solution was stirred at RT until the gas evolution had subsided and then was heated at 70° C. overnight. The cooled reaction was quenched carefully with 3M HCl (2 ml), then 70° C. to complete the quench. The reaction was cooled to RT and concentrated to a white solid, which was dissolved in 3M NaOH (pH 14) and extracted with CH₂Cl₂ (4×). The organics were dried (Na₂SO₄), filtered, and concentrated to provide 0.20 g (87%) of crude product as an oil, which was purified by precipitation from CH₂Cl₂ and 3M HCl/EtOAc (0.26 ml, 0.78 mmol) to yield 2-(3-nitrophenyl)ethanamine as the HCl salt as an off-white solid (0.164 g). ¹H NMR (DMSO-d₆): δ 8.18-8.15 (m, 1H), 8.13-8.04 (m, 1H), 8.02 (br s, 3H), 7.76-7.74 (m, 1H), 7.65 (br t, J=7.84 Hz), 3.17-3.08 (m, 2H), 3.06-3.00 (m, 2H); MS (ESI) m/z: 167.0 (M+H⁺).

To a stirring suspension of 2-(3-nitrophenyl)ethanamine hydrochloride (0.164 g, 0.81 mmol) in dry CH₂Cl₂ (8 ml) at RT was added DIEA (0.42 ml, 2.43 mmol). The reaction was stirred at RT until the solids were dissolved, then cooled thoroughly in an ice bath and TFAA (0.14 ml, 1.01 mmol) was added dropwise. The resulting yellow solution was stirred overnight with slow warning to RT. The reaction mixture was washed with ice H₂O (2×) and dried (MgSO₄). Filtration and evaporation provided N-(3-nitrophenethyl)-2,2,2-trifluoro-acetamide (0.215 g, 101% yield) of as an oil that solidified on standing. ¹H NMR (CDCl₃): δ 8.17-8.14 (m, 1H), 8.11-8.10 (m, 1H), 7.58-7.52 (m, 2H), 6.4 (brs, 1H), 3.70 (q, J=6.00 Hz, 2H), 3.06 (t, J=6.00 Hz, 2H).

To a solution of N-(3-nitrophenethyl)-2,2,2-trifluoroacetamide (9.05 g, 34.5 mmol) in MeOH (125 ml) at RT was added 10% Pd/C (50% water wet) (3.67 g, 1.73 mmol). The resulting suspension was placed under 3 atm of H₂ at 20-25° C. overnight. The reaction was filtered through Celite and the cake rinsed with MeOH. The filtrate was concentrated to provide N-(3-aminophenethyl)-2,2,2-trifluoroacetamide as an oil (7.83 g, 98% yield). ¹H NMR (CDCl₃): δ 7.16-7.12 (m, 1H), 6.62-6.58 (m, 2H), 6.54-6.53 (m, 1H), 6.34 (brs, 1H), 3.61 (q, J=6.40 Hz, 2H), 2.80 (t, J=6.40 Hz, 2H), 2.68 (brs, 2H); MS (ESI) m/z: 233.3 (M+H⁺).

To a stirring solution of N-(3-aminophenethyl)-2,2,2-trifluoroacetamide (7.83 g, 33.7 mmol) in EtOAc (80 ml) at RT was added 3M HCl/EtOAc (12.4 ml, 37.1 mmol). Solids precipitated almost immediately. The resulting suspension was cooled in ice 1 h. The solids were collected by filtration, rinsed with EtOAc and dried on the filter. There was obtained pure N-(3-aminophenethyl)-2,2,2-trifluoroacetamide hydrochloride free of less polar impurities as a pale tan solid (7.94 g, 88% yield). ¹H NMR (DMSO-d₆): δ 10.36 (br s, 3H), 9.61 (t, J=5.32 Hz, 1H), 7.43-7.39 (m, 1H), 7.25-7.23 (m, 2H), 3.42 (q, J=6.60 Hz, 2H), 2.84 (t, J=6.60 Hz, 2H).

N-(3-aminophenethyl)-2,2,2-trifluoroacetamide hydrochloride (0.27 g, 1.0 mmol) was suspended in 6M HCl (2.0 ml) and cooled thoroughly in an ice bath. This was rapidly stirred while a solution of sodium nitrite (73 mg) in H₂O (1.0 ml) was added slowly. The mixture was stirred at 0-5° C. for 45 min and was then treated with tin chloride dihydrate (1.3 g, 5.8 mmol) in 6M HCl (4.0 ml). The resulting suspension was stirred at 0-5° C. for 3 h and then carefully quenched with 3M NaOH (15 mL) to pH 7-8. The mixture was diluted with Et₂O, filtered through Celite and the filter cake was washed with H₂O and with Et₂O. The layers of the biphasic filtrate were separated and the aqueous extracted with Et₂O (2×). The combined organics extracts were washed with brine (1×), dried (Na₂SO₄), filtered and evaporated to provided N-(3-hydrazinophenethyl)-2,2,2-trifluoroacetamide as a pale yellow oil (0.18 g, 72% yield), which was used without further purification. MS (ESI) m/z: 248.0 (M+H⁺).

### Example A17

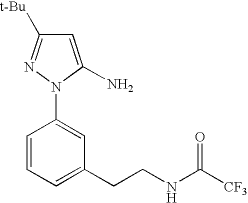

To a stirring solution of Example A16 (0.18 g, 0.73 mmol) in absolute EtOH (5 ml) at RT was added pivaloylacetonitrile (0.11 g, 0.87 mmol) and sat'd. HCl/EtOH (3 drops from a pipet). The resulting solution was stirred at 75-80° C. overnight, then cooled to RT and concentrated. The residue was dissolved in Et₂O and washed with sat'd. NaHCO₃. The aqueous was extracted with Et₂O (1×). The combined organics were washed with brine (1×) and dried (MgSO₄), filtered, concentrated and purified via flash chromatography to provide N-[3-(5-amino-3-t-butyl-1H-pyrazol-1-yl)phenethyl]-2,2,2-trifluoroacetamide as an orange glass (0.18 g, 70% yield). ¹H NMR (CDCl₃): δ 7.47-7.46 (m, 2H), 7.43-7.39 (m, 1H), 7.14-7.12 (m, 1H), 5.51 (s, 1H), 3.67 (q, J=6.48 Hz, 2H), 2.95 (t, J=6.48 Hz, 2H), 1.33 (s, 9H); MS (ESI) m/z: 355.2 (M+H⁺).

### Example 370

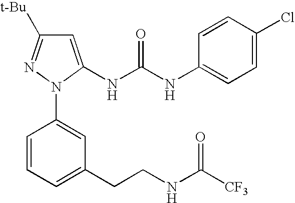

To a stirring solution of Example A17 (0.180 g, 0.51 mmol) in dry CH₂Cl₂ (5 ml) at RT was added 4-chlorophenyl isocyanate (82 mg, 0.53 mmol). The resulting mixture was stirred at RT overnight. More 4-chlorophenyl isocyanate was added (40 mg, 0.26 mmol) and stirring was continued. After 2 h, the reaction was concentrated to dryness and purified by flash chromatography to yield pure 1-(3-t-butyl-1-{3-[2-(2,2,2-trifluoroacetamido)ethyl]-phenyl}-1H-pyrazol-5-yl)-3-(4-chlorophenyl)urea as an orange foam (0.134 g, 52% yield). ¹H NMR (CDCl₃): δ 8.14 (br s, 1H), 7.39-7.20 (m, 8H), 7.03 (br s, 1H), 6.57 (s, 1H), 3.77 (m, 2H), 2.88 (m, 2H), 1.35 (s, 9H); MS (ESI) m/z: 508.3 (M+H⁺).

### Example 371

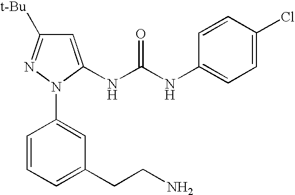

To a stirring solution of Example 370 (0.134 g, 0.264 mmol) in MeOH (10 ml) and H₂O (0.6 ml) at RT was added potassium carbonate (0.182 g, 1.32 mmol). The resulting suspension was stirred at 60-65° C. for 2 h, then cooled to RT and the volatiles evaporated. The residue was carefully dissolved in 1M HCl to pH 1-2 and extracted with Et₂O (2×). The aqueous was then basified (pH 13-14) with 3M NaOH and extracted with CH₂Cl₂ (4×). The combined CH₂Cl₂ extracts were washed with brine (1×), dried (Na₂SO₄), filtered, and concentrated to provided 1-{1-[3-(2-aminoethyl)phenyl]-3-t-butyl-1H-pyrazol-5-yl}-3-(4-chlorophenyl)urea as a foam (70.6 mg, 65% yield). ¹H NMR (CDCl₃): δ 8.64 (br s, 1H), 7.33-7.00 (m, 8H), 6.39 (s, 1H), 2.65 (m, 4H), 1.31 (s, 9H); MS (ESI) m/z: 412.3 (M+H⁺).

### Example 372

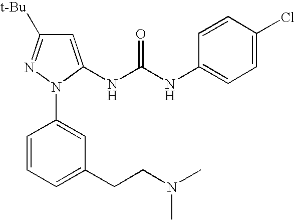

To a stirring solution of Example 371 (50 mg, 0.12 mmol) in MeOH (1.2 ml) at RT was added aq. formaldehyde (37 wt %, 0.036 ml, 0.49 mmol) and conc. formic acid (0.037 ml, 0.97 mmol). The reaction was stirred at 60-65° C. overnight, then cooled to RT, diluted with 1M HCl and filtered. The filtrate was made basic (pH 13) with 3M NaOH and extracted with CH₂Cl₂ (2×). The combined organics were washed with brine (1×), dried (Na₂SO₄), filtered, concentrated and purified by column chromatography, to yield 1-{3-t-butyl-1-[3-(2-(dimethylamino)ethyl]phenyl}-1H-pyrazol-5-yl)-3-(4-chlorophenyl)urea (12.5 mg, 23% yield) of product as a glass. ¹H NMR (CDCl₃): δ 8.33 (br s, 1H), 8.26 (br s, 1H), 7.43-7.06 (m, 8H), 6.51 (s, 1H), 2.84 (t, J=6.3 Hz, 2H), 2.75 (t, J=6.3 Hz, 2H), 2.27 (s, 6H), 1.36 (s, 9H); MS (ESI) m/z: 440.2 (M+H⁺).

### Example 373

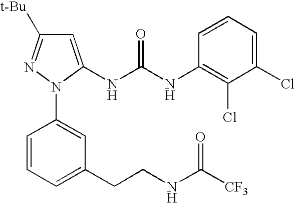

To a stirring solution of Example A17 (50 mg, 0.14 mmol) in dry THF (1.0 ml) at RT was added pyridine (0.11 ml, 1.4 mmol) followed by 2,3-dichlorophenyl isocyanate (0.037 ml, 0.28 mmol). The reaction was stirred overnight at RT, then diluted with 1M HCl (10 ml) and stirred for 1 h. The mixture was extracted with EtOAc (3×). The combined organic extracts were washed with H₂O (1×), satd. NaHCO₃ (1×), brine (1×), then dried (MgSO₄) filtered, concentrated and purified via column chromatography to provide 1-(3-t-butyl-1-{3-[2-(2,2,2-trifluoroacetamido)ethyl]phenyl}-1H-pyrazol-5-yl)-3-(2,3-dichlorophenyl)urea (32.2 mg, 42% yield). ¹H NMR (CDCl₃): δ 8.19(dd, J=1.92, 7.92 Hz, 1H), 8.02 (br s, 1H), 7.88 (br s, 1H), 7.45-7.36 (m, 3H), 7.22-7.15 (m, 3H), 7.05 (br d, J=7.44 Hz, 1H), 6.59 (s, 1H), 3.78 (q, J=6.44 Hz, 2H), 2.90 (t, J=6.4 Hz, 2H), 1.37 (s, 9H); (ESI) m/z: 542.3 (100, M+H⁺), 543.2 (30, M+2), 544.2 (66, M+3).

### Example 374

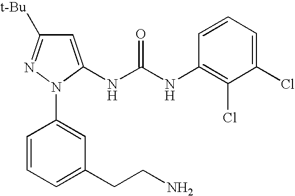

To a stirring solution of Example 373 (32.2 mg, 0.059 mmol) in MeOH (1.80 ml) and H₂O (0.15 ml) at RT was added potassium carbonate (41.0 g, 0.297 mmol). The resulting suspension was stirred at 60-65° C. for 2 h. The reaction was cooled to RT, diluted with H₂O and extracted with CHCl₃ (3×). The combined organics were washed with brine (1×), dried (Na₂SO₄), filtered and concentrated to provide 1-{1-[3-(2-aminoethyl)phenyl]-3-t-butyl-1H-pyrazol-5-yl}-3-(2,3-dichlorophenyl)urea as a waxy solid (25.6 mg, 97% yield). ¹H NMR (CDCl₃): δ 8.17 (dd, J=1.24, 8.08 Hz, 1H), 7.31-7.28 (m, 4H), 7.14-7.06 (m, 4H), 6.45 (s, 1H), 3.48 (br t, J=4.4 Hz, 2H), −3.46-3.39 (m, 2H), 2.86 (t, J=7.0 Hz, 2H), 1.3 (s, 9H).

### Example 375

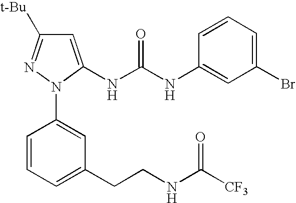

Using the same procedure as for Example 373, Example A17 (50 mg, 0.14 mmol) and 3-bromophenyl isocyanate (0.035 ml, 0.28 mmol) were combined to afford 1-(3-bromophenyl)-3-(3-t-butyl-1-{3-[2-(2,2,2-trifluoroacetamido)ethyl]phenyl}-1H-pyrazol-5-yl)urea (20.6 mg, 26% yield). ¹H NMR (CDCl₃): δ 8.17 (s, 1H), 7.66 (t, J=1.76 Hz, 1H), 7.49 (t, J=6.48 Hz, 1H), 7.42 (s, 1H), 7.37-7.34 (m, 3H), 7.23-7.20 (1H), 7.17-7.05 (m, 3H), 6.58 (s, 1H), 3.78 (q, J=6.4 Hz, 2H), 2.89 (t, J=6.1 Hz, 2H), 1.36 (s, 9H); MS (ESI) m/z: 552.2 (100, M+H⁺), 554.2 (98, M+2).

### Example 376

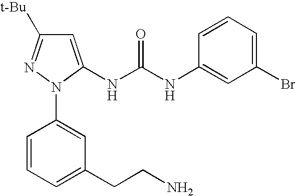

Using the same procedure as for Example 374, Example 375 (20.6 mg, 0.037 mmol) was deprotected to provide 1-{1-[3-(2-aminoethyl)phenyl]-3-t-butyl-1H-pyrazol-5-yl}-3-(3-bromophenyl)urea (22.4 mg). ¹H NMR (CDCl₃): δ 8.30 (br s, 1H), 7.53 (br s, 1H), 7.32-7.0 (m, 8H), 6.41. (s, 1H), 3.0-2.7 (br s, 4H), 1.34 (s, 9H); MS (ESI) m/z: 456.2 (100, M+H⁺), 458.2 (98, M+2).

### Example 377

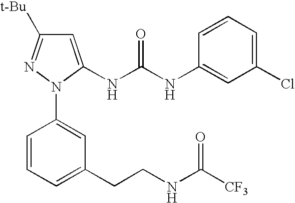

Using the same procedure as for Example 373, Example A17 (50 mg, 0.14 mmol) and 3-chlorophenyl isocyanate (0.034 ml, 0.28 mmol) were combined to afford 1-(3-t-butyl-1-{3-[2-(2,2,2-trifluoroacetamido)ethyl]phenyl}-1H-pyrazol-5-yl)-3-(3-chlorophenyl)urea (32.2 mg, 45% yield). ¹H NMR (CDCl₃): δ 8.18 (s, 1H), 7.51-7.48 (m, 2H), 7.43 (s, 1H), 7.37-7.34 (m, 3H), 7.20-7.14 (m, 2H), 7.08-7.05 (m, 1H), 7.02-6.99 (m, 1H), 6.58 (s, 1H), 3.78 (q, J=6.4 Hz, 2H), 2.88 (t, J=6.4 Hz, 2H), 1.36 (s, 9H); MS (ESI) m/z: 508.3 (100, M+H⁺), 510.2 (37, M+2).

### Example 378

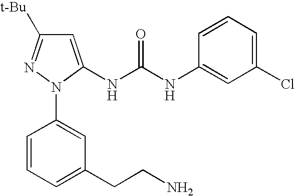

Using the same procedure as for Example 374, Example 377 (32.2 mg, 0.063 mmol) was deprotected to afford 1-{1-[3-(2-aminoethyl)phenyl]-3-t-butyl-1H-pyrazol-5-yl}-3-(3-chlorophenyl)urea (19.1 mg, 73% yield). ¹H NMR (CDCl₃): δ 8.29 (br s, 1H), 7.46 (s, 1H), 7.43-7.29 (m, 1H), 7.23-7.19 (m, 2H), 7.16-7.10 (m, 3H), 7.01-6.97 (m, 2H), 6.41 (s, 1H), 2.94 (br s, 2H), 2.71 (br s, 2H), 1.34 (s, 9H); MS (ESI) m/z: 412.3 (100, M+H⁺), 414.2 (36, M+2).

### Example 379

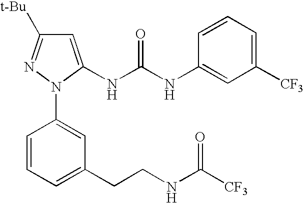

Using the same procedure as for Example 373, Example A17 (50 mg, 0.14 mmol) and α,α,α-trifluoro-m-tolyl isocyanate (0.039 ml, 0.28 mmol) were combined to provide 1-(3-t-butyl-1-{3-[2-(2,2,2-trifluoroacetamido)ethyl]phenyl}-1H-pyrazol-5-yl)-3-[3-(trifluoro methyl)phenyl]urea (31.1 mg, 41% yield). ¹H NMR (CDCl₃): δ 8.23 (s, 1H), 7.66 (s, 1H), 7.61-7.59 (m, 1H), 7.42-7.36 (m. 4H), 7.27-7.26 (m, 1H), 7.12-7.09 (m, 1H), 7.08-7.05 (m, 1H), 6.64 (s, 1H), 3.88 (q, J=5.5 Hz, 2H), 2.95 (t, J=5.5 Hz, 2H), 1.37 (s, 9H); MS (ESI) m/z: 542.3 (M+H⁺).

### Example 380

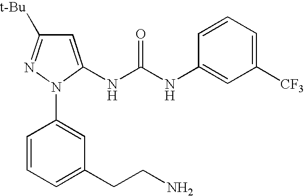

Using the same procedure as for Example 374, Example 379 (31.1 mg, 0.057 mmol) was deprotected to provide 1-{1-[3-(2-aminoethyl)phenyl]-3-t-butyl-1H-pyrazol-5-yl}-3-[3-(trifluoromethyl)phenyl]urea (24.8 mg, 97% yield). ¹H NMR (CDCl₃): δ 8.34 (brs, 1H), 7.60 (s, 1H), 7.54-7.50 (m, 1H), 7.37-7.37.25 (m. 5H), 7.18-7.17 (m, 1H), 6.44 (s, 1H), 2.99 (br s, 2H), 2.75 (br s, 2H), 1.35 (s, 9H); MS (ESI) m/z: 446.3 (M+H⁺).

### Example 381

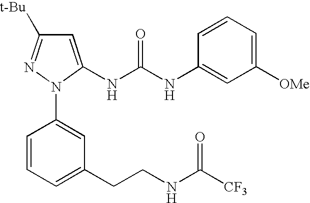

Using the same procedure as Example 373, Example A17 (50 mg, 0.14 mmol) and 3-methoxyphenyl isocyanate (0.037 ml, 0.28 mmol) were combined to afford 1-(3-t-butyl-1-{3-[2-(2,2,2-trifluoroacetamido)ethyl]phenyl}-1H-pyrazol-5-yl)-3-(3-methoxy phenyl)urea (29.6 mg, 42% yield). ¹H NMR (CDCl₃): δ 8.01 (s, 1H), 7.39-7.34 (m, 4H), 7.28-7.24 (m, 1H), 7.18-7.14 (m, 1H), 7.11-7.09 (m, 1H), 7.08-7.06 (m, 1H), 7.06-7.05 (m, 1H), 6.87-6.84 (m, 1H), 6.61-6.60 (m, 1H), 6.59 (s, 1H), 3.78 (q, J=6.6 Hz, 2H), 3.77 (s, 3H), 2.88 (t, J=6.6 Hz, 2H), 1.36 (s, 9H); MS (ESI) m/z: 504.2 (M+H⁺).

### Example 382

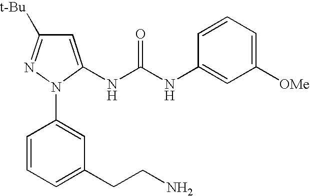

Using the same procedure as for Example 374, Example 381 (29.6 mg, 0.059 mmol) was deprotected to provide 1-{1-[3-(2-aminoethyl)phenyl]-3-t-butyl-1H-pyrazol-5-yl}-3-(3-methoxyphenyl)urea (16.4 mg, 69% yield). ¹H NMR (CDCl₃): δ 7.89 (br s, 1H), 7.34-7.27 (m, 4H), 7.16-7.13 (m, 2H), 7.06 (br s, 1H), 6.78-6.76 (m, 1H), 6.61-6.58 (m, 1H), 6.41 (s, 1H), 3.76 (s, 3H), 2.96 (br s, 2H), 2.75 (br s, 2H), 1.35 (s, 9H); MS (ESI) m/z: 408.3 (M+H⁺).

### Example 383

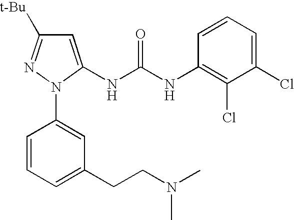

Using the same procedure as for Example 372, Example 374 (54.2 mg, 0.121 mmol) was obtained 1-(3-t-butyl-1-{3-[2-(dimethylamino)ethyl]phenyl}-1H-pyrazol-5-yl)-3-(2,3-di-chlorophenyl)urea (17.4 mg, 30% yield).

### Example 384

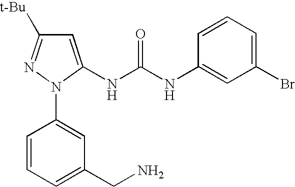

To a stirring solution of Example 356 (0.17 g, 0.39 mmol) in dry THF (4 ml) at RT was added 1.0M LAH in THF (0.58 ml, 0.58 mmol). After 2 h at RT additional 1.0M LiAlH4 in THF (0.58 ml, 0.58 mmol) was added and the reaction was stirred an 1 h. The reaction was carefully quenched by the addition of H₂O (0.044 ml), 3M NaOH (0.044 ml) and H₂O (0.088 ml) and stirred overnight at RT. The mixture was filtered through Celite, rinsing generously with EtOAc. The filtrate was concentrated to dryness to give 0.13 g of crude product, which was redissolved in EtOAc and treated with 3M HCl/EtOAc. A precipitate formed immediately, which was collected by filtration, rinsed with EtOAc and dried to yield 1-{1-[3-(aminomethyl)phenyl]-3-t-butyl-1H-pyrazol-5-yl}-3-(3-bromophenyl)urea as the HCl salt (0.131 g, 70% yield). ¹H NMR (DMSO-d₆): δ 9.93 (s, 1H), 8.83 (s, 1H), 8.36 (br s, 3H), 7.82-7.81 (m, 1H), 7.71 (br s, 1H), 7.57-7.55 (m, 2H), 7.48-7.46 (m, 1H), 7.31-7.29 (m, 1H), 7.24-7.20 (m, 1H), 7.15-7.13 (m, 1H), 6.42 (s, 1H), 4.16-4.12 (m, 2H), 1.29 (s, 9H); MS (ESI) m/z: 442.3 (M+H⁺), 444.2 (M+2+H⁺).

### Example 385

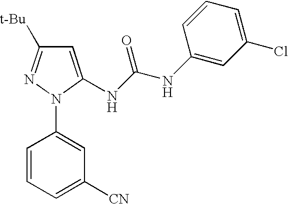

Using the same procedure as for Example 304, Example A13 (0.0500 g, 0.208 mmol) and 3-chlorophenyl isocyanate (0.0507 mL, 0.416 mmol) were combined to afford 1-(3-t-butyl-1-(3-cyanophenyl)-1H-pyrazol-5-yl)-3-(3-chlorophenyl)urea as an oil (32.8 mg, 40% yield)). ¹H NMR (CDCl₃): δ 7.79-7.76 (m, 2H), 7.60 (s, 1H), 7.48-7.44 (3H), 7.26-7.25 (m, 1H), 7.16-7.12 (m, 1H), 7.05-7.01 (m, 2H), 6.37 (s, 1H), 1.31 (s, 9H); MS (ESI) m/z: 394.2 (M+H⁺), 396.3 (M+2+H⁺).

### Example 386

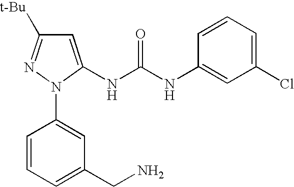

Using the same procedure as for Example 384, Example 112 (0.11 g, 0.28 mmol) was reduced to afford 1-{1-[3-(aminomethyl)phenyl]-3-t-butyl-1H-pyrazol-5-yl}-3-(3-chloro-phenyl)urea as an off-white HCl salt (77.2 mg, 64% yield). ¹H NMR (DMSO-d₆): δ 10.11 (s, 1H), 8.91 (s, 1H), 8.43 (br s, 3H), 7.72 (s, 1H), 7.68 (s, 1H), 7.56-7.55 (m, 2H), 7.48-7.46 (m, 1H), 7.31-7.25 (m, 2H), 7.02-6.99 (m, 1H), 6.42 (s, 1H), 4.16-4.12 (m, 2H), 1.30 (s, 9H); MS (ESI) m/z: 398.3 (M+H⁺), 400.2 (M+2+H⁺).

### Example 387

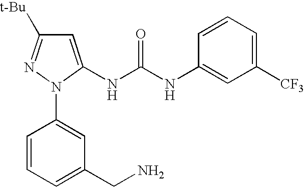

Using the same procedure as for Example 384, Example 361 (0.120 g, 0.28 mmol) was reduced to afford 1-{1-[3-(aminomethyl)phenyl]-3-t-butyl-1H-pyrazol-5-yl}-3-(3-(trifluoro-methyl)phenyl)urea as an off-white HCl salt (73.9 mg, 56% yield). ¹H NMR (DMSO-d₆): δ 10.26 (s, 1H), 8.94 (s, 1H), 8.42 (br s, 3H), 7.98 (s, 1H), 7.73 (s, 1H), 7.58-7.47 (m, 5H), 7.32-7.30 (m, 1H), 6.44 (s, 1H), 4.14 (m, 2H), 1.29 (s, 9H); MS (ESI) m/z: 432.2 (M+H⁺)

### Example 388

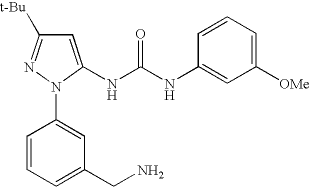

Using the same procedure as for Example 384, Example 360 (0.16 g, 0.411 mmol) was reduced to afford 1-{1-[3-(aminomethyl)phenyl]-3-t-butyl-1H-pyrazol-5-yl}-3-(3-methoxy-phenyl)urea as an off-white HCl salt (137 mg, 77% yield). ¹H NMR (DMSO-d₆): δ 9.75 (s, 1H), 8.80 (s, 1H), 8.43 (br s, 3H), 7.72 (s, 1H), 7.56-7.55 (m, 2H), 7.49-7.47 (m, 1H), 7.18-7.13 (m, 2H), 6.92-6.89 (m, 1H), 6.55-6.53 (m, 1H), 6.41 (s, 1H), 4.16-4.12 (m, 2H), 3.71 (s, 3H), 1.29 (s, 9H); MS (ESI) m/z: 394.2 (M+H⁺).

### Example 389

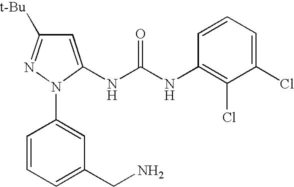

Using the same procedure as for Example 384, Example 359 (50 mg, 0.12 mmol) was reduced to afford 1-{1-[3-(aminomethyl)phenyl]-3-t-butyl-1H-pyrazol-5-yl}-3-(2,3-dichloro-phenyl)urea as a white solid (20.6 mg, 41% yield). ¹H NMR (CDCl₃): δ 9.55 (s, 1H), 8.47 (br s, 3H), 7.97-7.96 (m, 1H), 7.70-7.32 (m, 4H), 7.15-7.11 (m, 3H), 6.81 (s, 1H), 4.10 (br s, 2H), 1.38 (s, 9H); MS (ESI) m/z: 432.2 (M+H⁺), 434.2 (M+2+H⁺).

### Example 390

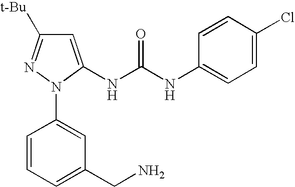

Using the same procedure as for Example 384, Example 358 (87 mg, 0.22 mmol) was reduced to afford 1-{1-[3-(aminomethyl)phenyl]-3-t-butyl-1H-pyrazol-5-yl}-3-(4-chloro-phenyl)urea as the HCl salt (78 mg, 82% yield). ¹H NMR (DMSO-d₆): δ 9.96 (s, 1H), 8.85 (s, 1H), 8.42 (br s, 3H), 7.72 (s, 1H), 7.56-7.55 (m, 2H), 7.48-7.45 (m, 3H), 7.32-7.30 (m, 2H), 6.41 (s, 1H), 4.16-4.12 (m, 2H), 1.29 (s, 9H); MS (ESI) m/z: 398.3 (M+H⁺), 400.2 (M+2+H⁺).

### Example 391

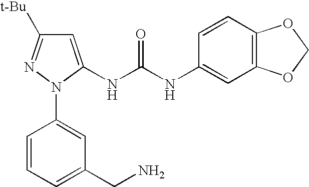

Using the same procedure as for Example 384, Example 357 (0.100 g, 0.25 mmol) was reduced to afford 1-{1-[3-(aminomethyl)phenyl]-3-t-butyl-1H-pyrazol-5-yl}-3-(benzo-[d][1,3]dioxol-5-yl)urea as its TFA salt as a white powder (67.1 mg, 66% yield). ¹H NMR (DMSO-d₆): δ 8.96 (s, 1H), 8.41 (s, 1H), 8.19 (br s, 3H), 7.67-7.47 (m, 4H), 7.15 (s, 1H), 6.82-6.80 (m, 1H), 6.71-6.69 (m, 1H), 6.37 (s, 1H), 5.96 (s, 2H), 4.13-4.12 (m 2H), 1.28 (s, 9H); MS (ESI) m/z: 408.3 (M+H⁺).

### Example 392

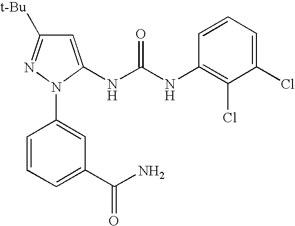

Example 359 (80 mg, 0.19 mmol) was suspended in conc. HCl (0.93 ml) and briskly stirred. More conc. HCl (1 ml) was added several times to maintain good stirring and keep the solids wetted. The reaction was stirred at RT 5 h and 24 h at 40° C. The reaction was cooled to RT, diluted with H₂O and EtOAc and the layers separated. The aqueous was extracted with EtOAc (2×). Solids in the aqueous layer were collected by filtration, rinsed sparingly with H₂O and dried. These solids were suspended in MeOH, then collected by filtration, rinsed with MeOH and washed with EtOAc to afford 1-[3-t-butyl-1-(3-carbamoylphenyl)-1H-pyrazol-5-yl]-3-(2,3-dichlorophenyl)urea as a white solid (47.3 mg, 57% yield). ¹H NMR (DMSO-d₆): δ 9.81 (br s, 1H), 8.99 (br s, 1H), 8.25 (br s, 1H), 8.08 (s, 1H), 7.99-7.97 (m, 1H), 7.90-7.87 (m, 1H), 7.75-7.71 (m, 1H), 7.60-7.57 (m, 1H), 7.49 (br s, 1H), 7.32-7.28 (m, 2H), 6.38 (br s, 1H), 1.29 (s, 9H); MS (ESI) m/z: 446.3 (M+H⁺),448.3 (M+2+H⁺).

### Example 393

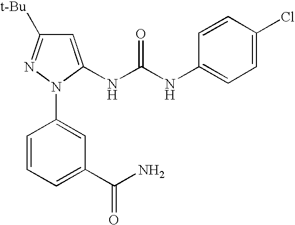

Using the same procedure as Example 392, Example 358 (0.174 g, 0.442 mmol) was transformed to provide 1-[3-t-butyl-1-(3-carbamoylphenyl)-1H-pyrazol-5-yl]-3-(4-chlorophenyl)urea as a pale yellow fluffy solid (47.4 mg). ¹H NMR (DMSO-d₆): δ 9.13 (s, 1H), 8.51 (s, 1H), 8.11 (br s, 1H), 8.02-8.01 (m, 1H), 7.92-7.89 (m, 1H), 7.68-7.66 (m, 1H), 7.62-7.58 (m, 1H), 7.52 (br s, 1H), 7.44-7.42 (m, 2H), 7.31-7.29 (m, 2H), 6.39 (s, 1H), 1.29 (s, 9H; MS (ESI) m/z: 412.3 (M+H⁺), 414.2(M+2+H⁺).

### Example 394

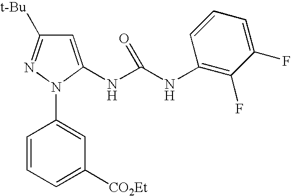

Using the same procedure as for Example 305, Example A2 (143 mg, 0.5 mmol) and 2,3-difluorophenylamine (67 mg, 0.5 mmol) were combined to afford ethyl 3-{3-t-butyl-5-[3-(2,3-difluorophenyl)ureido]-1H-pyrazol-1-yl}benzoate (50 mg, 23% yield).

### Example 395

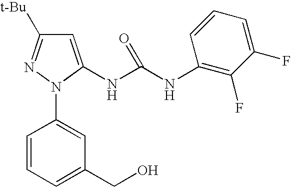

Using the same procedure as for Example 303, Example 394 (45 mg, 0.10 mmol) was reduced to afford 1-{3-t-butyl-1-[3-(hydroxymethyl)phenyl]-1H-pyrazol-5-yl}-3-(2,3-difluorophenyl)urea (30 mg, 75% yield). ¹H-NMR (300 MHz, DMSO-d₆): δ 9.08 (s, 1H), 8.85 (s, 1H), 7.88 (t, J=7.5 Hz, 1H), 7.48-7.42 (m, 2H), 7.33 (d, J=7.5 Hz, 2H), 7.13-6.95 (m, 2H), 6.36 (s, 1H), 4.55 (s, 1H), 1.24 (s, 9H); MS (ESI) m/z: 401 (M+H⁺).

### Example A18

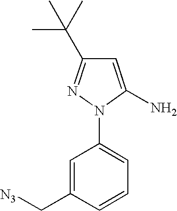

To a suspension of LiAlH₄ (5.28 g, 139.2 mmol) in THF (1000 mL) was added Example A2 (20.0 g, 69.6 mmol) in portions at 0° C. under N₂. The reaction mixture was stirred for 5 h, quenched with 1 N HCl at 0° C. and the precipitate was filtered, washed by EtOAc and the filtrate evaporated to yield [3-(5-amino-3-t-butyl-1H-pyrazol-1-yl)phenyl]methanol (15.2 g, 89%). ¹H NMR (DMSO-d₆): 7.49 (s, 1H), 7.37 (m, 2H), 7.19 (d, J=7.2 Hz, 1H), 5.35 (s, 1H), 5.25 (t, J=5.6 Hz, 1H), 5.14 (s, 2H), 4.53 (d, J=5.6 Hz, 2H), 1.19 (s, 9H); MS (ESI) m/z: 246.19 (M+H⁺).

The crude material from the previous reaction (5.0 g, 20.4 mmol) was dissolved in dry THF (50 mL) and SOCl₂ (4.85 g, 40.8 mmol), stirred for 2 h at RT, concentrated in vacuo to yield 3-t-butyl-1-(3-chloromethylphenyl)-1H-pyrazol-5-amine (5.4 g), which was added to NaN₃ (3.93 g, 60.5 mmol) in DMF (50 mL). The reaction mixture was heated at 30° C. for 2 h, poured into H₂O (50 mL), and extracted with CH₂Cl₂. The organic layers were combined, dried (MgSO₄), filtered and concentrated in vacuo to yield crude 3-t-butyl-1-[3-(azidomethyl)phenyl]-1H-pyrazol-5-amine (1.50 g, 5.55 mmol).

### Example 396

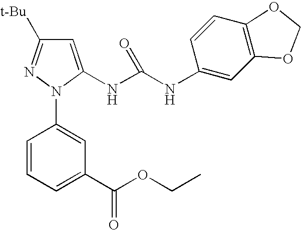

Using the same procedure as for Example 304, Example A2 (500 mg, 1.74 mmol) and 5-isocyanato-benzo[1,3]dioxole (290 mg, 1.8 mmol) were combined to afford ethyl 3-{5-[3-(benzo[d][1,3]dioxo-5-yl)ureido]-3-t-butyl-1H-pyrazol-1-yl}benzoate (320 mg, 41% yield). ¹H NMR (300 MHz, DMSO-d₆): δ 8.73 (s, 1H), 8.34 (s, 1H), 8.03 (s,1H), 7.92 (d, J=8.4 Hz, 1H), 7.78 (d, J=7.8 Hz, 1H), 7.63 (t, J=7.8 Hz, 1H), 7.09 (s, 1H), 6.76 (d, J=8.1 Hz, 2H), 6.68 (d, J=8.4 Hz, 1H), 6.32 (s, 1H), 5.92 (s, 2H), 4.29 (q, J=6.9 Hz, 2H), 1.28 (s, 9H), 1.26 (t, J=6.9 Hz, 3H); MS (ESI) m/z: 451 (M+H⁺).

### Example 397

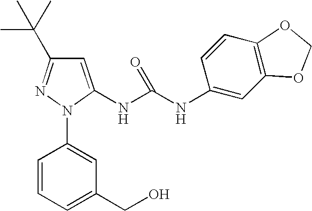

Using the same procedure as for example 303, Example 396 (100 mg, 0.22 mmol) was reduced to afford 1-(benzo[d][1,3]dioxol-5-yl)-3-(3-t-butyl-1-(3-(hydroxymethyl)phenyl)-1H -pyrazol-5-yl)urea (50 mg, 56% yield). ¹H NMR (300 MHz, CD₃OD): δ 7.52-7.47 (m, 4H), 7.02 (s, 1H), 6.65-6.69 (m, 2H), 6.41 (s, 1H), 5.89 (s, 2H), 4.69 (s, 2H), 1.33 (s, 9H); MS (ESI) m/z: 409 (M+H⁺).

### Example 398

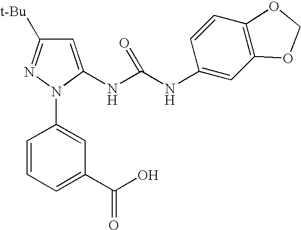

To a solution of Example 396 (50 mg, 0.11 mmol) in THF (10 mL) was added a aqueous solution of LiOH (2 N, 5 mL) at 0° C. The mixture was stirred at RT overnight. After removal of the solvent, the residue was dissolved in water, then acidified to pH=4.0 with 1 N of HCl. The mixture was extracted with CH₂Cl₂ (3×50 mL) and t the combined organic extracts were washed with brine, dried (Na₂SO₄), filtered, concentrated and purified via preparative HPLC to afford 3-{5-[3-(benzo[d][1,3]dioxol-5-yl)ureido]-3-t-butyl-1H-pyrazol-1-yl}benzoic acid (30 mg, 65% yield). ¹H NMR (300 MHz, CD₃OD): δ 8.15 (s, 1H), 8.08 (d, J=7.8 Hz, 1H), 7.75 (d, J=8.4 Hz, 1H), 7.63 (t, J=7.8 Hz, 1H), 6.99 (s, 1H), 6.67-6.62 (m, 2H), 6.39 (s, 1H), 5.89 (s, 2H), 1.33 (s, 9H); MS (ESI) m/z: 423 (M+H⁺).

### Example A19

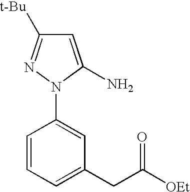

A mixture of 1-(3-nitro-phenyl)-ethanone (82.5 g, 0.5 mol), toluene-4-sulfonic acid (3 g) and sulfur (32 g, 1.0 mol) in morpholine (100 mL) was heated to reflux for 3 h. After removal of the solvent, the residue was dissolved in dioxane (100 mL), treated with conc. HCl (100 mL), then heated to reflux for 5 h. After removal of the solvent, the residue was extracted with EtOAc (3×150 mL). The combined organic layers were washed with brine, dried over anhydrous sodium sulfate and filtered. The filtrate was evaporated to the residue under reduced pressure. The residue was dissolved in ethanol (250 mL) and then added SOCl2 (50 mL). The mixture was heated to reflux for 2 h. After removal of the solvent, the residue was extracted with ethyl acetate (3×150 mL). The combined organic extracts were washed with brine, dried (Na₂SO₄), filtered, concentrated and purified via column chromatography to afford 40 g of (3-nitro-phenyl)-acetic acid ethyl ester. ¹H-NMR (300 MHz, DMSO-d6): δ 8.17 (s, 1H), 8.11 (d, J=7.2 Hz, 1H), 7.72 (d, J=7.2 Hz, 1H), 7.61 (t, J=7.8 Hz, 1H), 4.08 (q, J=7.2 Hz, 2H), 3.87 (s, 2H), 1.17 (t, J=7.2 Hz, 3H).

A mixture of (3-nitro-phenyl)-acetic acid ethyl ester (31.4 g, 0.15 mol) and Pd/C (3.5 g) in methanol (200 mL) was stirred under 40 psi of H₂ a tRT for 2 h, then filtered. After removal of the solvent, 25 g of 3-(3-amino-phenyl)-acetic acid ethyl ester was obtained (93%), which was used without further purification. MS (ESI): m/z: 180 (M+H⁺)

To a solution of 3-(3-amino-phenyl)-acetic acid ethyl ester (18 g, 0.1 mol) in concentrated HCl (200 mL) was added an aqueous solution (20 mL) of NaNO₂ (6.9 g, 0.1 mmol) at 0° C. and the resulting mixture was stirred for 1 h. A solution of SnCl₂.2H₂O (44.5 g, 0.2 mmol) in concentrated HCl (200 mL) was then added at 0° C. The reaction solution was stirred for 2 h at RT. The reaction mixture was adjusted to pH=8.0 with 2 N NaOH and extracted with EtOAc (3×150 mL). The combined organic extracts were washed with brine, dried (Na₂SO₄), filtered, and concentrated to yield 15 g of 3-(3-hydrazino-phenyl)-acetic acid ethyl ester (77%) as a white solid, which was used without further purification; MS(ESI) m/z: 194 (M+H⁺).

A mixture of 3-(3-hydrazino-phenyl)-acetic acid ethyl ester (9.7 g, 50 mmol) and 4,4-dimethyl-3-oxo-pentanenitrile (6.9 g, 55 mol) in ethanol (150 mL) was heated to reflux overnight. The reaction solution was evaporated under vacuum. The residue was purified via column chromatography to give 13 g 3-[3-(5-amino-3-t-butyl-pyrazol-1-yl)-phenyl]-acetic acid ethyl ester (87%). ¹H-NMR (300 MHz, DMSO-d₆); 7.44 (s, 1H), 7.43 (d, J=8.1 Hz, 1H), 7.35 (t, J=7.5 Hz, 1H), 7.12 (d, J=7.5 Hz, 1H), 5.35 (s, 1H), 5.17 (br s, 2H), 4.05 (q, J=6.9 Hz, 2H), 3.69{(s, 2-H), 1.18 (s, 9H), 1.16 (t, J=6.9 Hz, 3H); MS (ESI) m/z: 302 (M+H⁺)

### Example 399

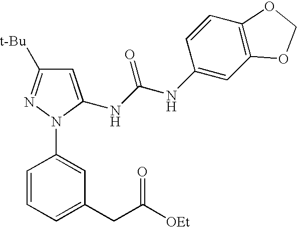

To a solution of Example A19 (1.0 g, 3.32 mmol) and triethylamine (606 mg, 6.0 mmol) in THF (50 mL) was added 5-isocyanato-benzo[1,3]dioxole (570 mg, 3.5 mmol) in THF (5.0 mL) at 0° C. The mixture was stirred at RT for 3 h, and then poured into water (100 mL). The mixture was extracted with CH₂Cl₂ (3×). The combined organic extracts were washed with brine, dried (Na₂SO₄), filtered, concentrated and purified via column chromatography to afford ethyl 2-(3-{5-[3-(benzo[d][1,3]dioxol-5-yl)ureido]-3-t-butyl-1H-pyrazol-1-yl}phenyl)-acetate (950 mg, 62% yield). ¹H NMR (300 MHz, DMSO-d₆): δ 8.84 (s, 1H), 8.28 (s, 1H), 7.48-7.34 (m, 3H), 7.27 (d, J=8.4 Hz, 1H), 7.11 (s, 1H), 6.76 (d, J=7.8 Hz, 1H), 6.66 (d, J=7.8 Hz, 1H), 6.31 (s, 1H), 5.92 (s, 2H), 4.04 (q, J=7.2 Hz, 2H), 3.73 (s, 2H), 1.23 (s, 9H), 1.15 (t, J=7.8 Hz, 3H); MS (ESI) m/z: 465 (M+H⁺).

### Example 400

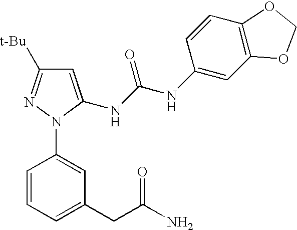

To a solution of Example 399 (150 mg, 0.20 mmol) in THF (5 mL) was added aqueous NH₃ (10 mL, 25%) at RT. The mixture was heated to 70° C. for 5 h, and then concentrated, and the residue was purified by preparative HPLC to afford 1-{1-[3-(2-amino-2-oxoethyl)phenyl]-3-t-butyl-1H-pyrazol-5-yl}-3-(benzo[d][1,3]dioxol-5-yl)urea (70 mg, 80% yield). ¹H-NMR (300 MHz, DMSO-d₆): δ 8.85 (s, 1H), 8.31 (s, 1H), 7.51-7.26 (m, 5H), 7.11 (s, 1H), 6.90 (br s, 1H), 6.76 (d, J=8.4 Hz, 1H), 6.65 (d, J=7.8 Hz, 1H), 6.32 (s, 1H), 5.92 (s, 2H), 3.42 (s, 2H), 1.23 (s, 9H); MS (ESI) m/z: 436 (M+H⁺).

### Example 401

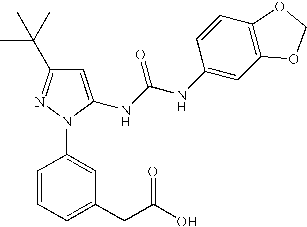

Using the same procedure as for Example 306, Example 399 (500 mg, 1.1 mmol) was saponified to afford 2-(3-{5-[3-(benzo[1,3]dioxol-5-yl)ureido]-3-t-butyl-1H-pyrazol-1-yl}phenyl)acetic acid (450 mg, 94% yield). ¹H NMR (300 MHz, CD₃OD): δ 7.52-7.35 (m, 4H), 7.01 (s, 1H), 6.70-6.61 (m, 2H), 6.40 (s, 1H), 5.89 (s, 2H), 3.72 (s, 2H), 1.32 (s, 9H); MS (ESI) m/z: 437 (M+H⁺).

### Example 402

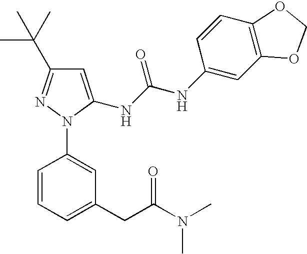

A mixture of Example 401 (500 mg, 1.1 mmol), dimethylamine (135 mg, 3.0 mmol), DIEA (390 mg, 3.0 mmol) and PyBop (780 mg, 1.5 mmol) in THF (50 mL) was stirred at RT overnight. After removal of the solvent, the residue was dissolved in CH₂Cl₂ (100 mL) and washed with 1.0 N HCl and brine. The combined organic extracts were washed with brine, dried (Na₂SO₄), filtered, concentrated and purified via column chromatography to afford 1-(benzo[d][1,3]dioxol-5-yl)-3-{3-t-butyl-1-[3-(2-(dimethylamino)-2-oxoethyl]phenyl}-1H-pyrazol-5-yl)urea (470 mg, 92% yield). ¹H NMR (300 MHz, DMSO-d₆): δ 9.00 (s, 1H), 8.39 (s, 1H), 7.43-7.35 (m, 3H), 7.21 (d, J=7.2 Hz, 1H), 7.11 (s, 1H), 6.76 (d, J=8.4 Hz, 1H), 6.65 (d, J=7.8 Hz, 1H), 6.30 (s, 1H), 5.92 (s, 2H), 3.73 (s, 2H), 2.98 (s, 3H), 2.78 (s, 3H), 1.23 (s, 9H); MS (ESI) m/z: 464 (M+H⁺).

### Example 403

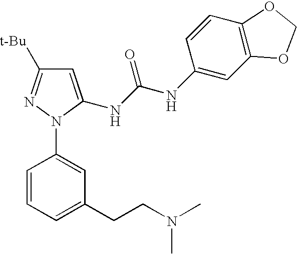

To a solution of Example 402 (150 mg, 0.32 mmol) in THF (20 mL) was added LAH powder (23 mg, 0.6 mmol) at RT under N₂. The mixture was heated to reflux for 3 h, and then quenched with water and aqueous NaOH. The suspension was filtered and the filtrate was concentrated and purified by preparative HPLC to afford the TFA salt. The mixture of TFA salt in MeCN/H₂O (50 mL) was basified to pH=10.0 with an aqueous solution of 1.0 N Na₂CO₃. After lyophylization, the residue was dissolved in THF, filtered and the filtrate was adjusted to pH=6.0 with 1.0 N HCl/MeOH (2.0 mL) and then concentrated to afford 1-(benzo[d][1,3]dioxol-5-yl)-3-(3-t-butyl-1-{3-[2-(dimethylamino)ethyl]phenyl}-1H-pyrazol-5-yl)urea (95 mg, 66% yield). ¹H NMR (300 MHz, CD₃OD): δ 7.56-7.39 (m, 4H), 6.99 (s, 1H), 6.71 (d, J=8.4 Hz, 1H), 6.62 (d, J=7.8 Hz, 1H), 6.38 (s, 1H), 5.89 (s, 2H), 3.41 (t, J=7.2 Hz, 2H), 3.13 (t, J=7.2 Hz, 2H), 2.90 (s, 6H), 1.34 (s, 9H); MS (ESI) m/z: 450 (M+H⁺).

### Example 404

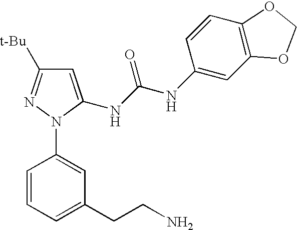

Using the same procedure as for Example 384, Example 400 (200 mg, 0.45 mmol) was reduced to yield 1-{1-[3-(2-aminoethyl)phenyl]-3-t-butyl-1H-pyrazol-5-yl}-3-(benzo[d][1,3]dioxol-5-yl)urea as the hydrochloride salt (80 mg, 40% yield). ¹H NMR (300 MHz, DMSO-d₆): δ 9.37 (s, 1H), 8.65 (s, 1H), 7.92 (br s, 3H), 7.52-7.47 (m, 3H), 7.28 (d, J=7.8 Hz, 1H), 7.02 (s, 1H), 6.65-6.69 (m, 2H), 6.31 (s, 1H), 5.92 (s, 2H), 3.13-3.07 (m, 2H), 2.96-2.88 (m, 2H), 1.24 (s, 9H); MS (ESI) m/z: 422 (M+H⁺).

### Example A20

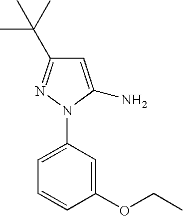

m-Phenetodine (1.51 g, 11.0 mmol) was dissolved in concentrated HCl (16 mL) and the solution was stirred in an ice-salt bath. Sodium nitrite (0.76 g, 11.0 mmol) was dissolved in water (14 mL) and chilled to 0° C., then added dropwise maintaining an internal reaction temperature of 0° C. The reaction mixture was stirred at 0-5° C. for 1 h. Reducing agent, tin chloride dehydrate (5.71 g, 25.3 mmol) was dissolved in conc. HCl (10 mL) and chilled to 0° C. and slowly added to the reaction mixture and stirred at 0-5° C. for 1 h. The reaction mixture was filtered and the solid washed with chilled 6N HCl. The solid was dissolved in water and lyophilized under reduced pressure to obtain 1-(3-ethoxyphenyl)-hydrazine HCl salt as a brown powder (1.61 g, 77% yield), which was used without further purification.

To a solution of 1-(3-ethoxyphenyl)-hydrazine (300 mg, 1.6 mmol) in toluene (5 mL) was added pivaloylacetonitrile (200 mg, 1.6 mmol). The reaction mixture was heated to reflux for 5 h. The reaction mixture was filtered and washed with hexane to obtain 3-t-butyl-1-(3-ethoxyphenyl)-1H-pyrazole-5-amine HCl salt as an orange solid (320 mg, 68% yield). ¹H NMR (DMSO-d₆): δ 7.47 (t, J=8.0 Hz, 1H), 7.0-7.4 (m, 3H), 5.60 (s, 1H), 4.12 (q, J=7.0 Hz, 2H), 1.35 (t, J=7.0 Hz, 3H), 1.28 (s, 9H); MS (EI) m/z: 260 (M+H⁺).

### Example 405

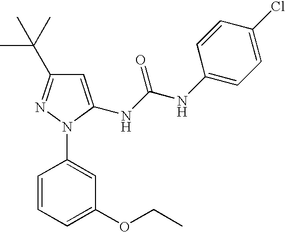

To a solution of Example A20 (70 mg, 0.14 mmol) in THF (2 mL) was added pyridine (38 mL, 0.28 mmol) and 4-chlorophenyl isocyanate (36 mg, 0.14 mmol). The reaction mixture was stirred at 40° C. for 12 h. The reaction mixture was concentrated under reduced pressure and the residue was purified by column chromatography to yield 1-[3-t-butyl-1-(3-ethoxyphenyl)-1H-pyrazol-5-yl]-3-(4-chlorophenyl)urea as a white powder (10 mg, 10% yield). ¹H NMR (CDCl₃): δ 7.34 (br s, 1H), 7.21 (m, 5H), 6.93 (m, 2H), 6.88 (br s, 1H), 6.83 (dd, J=1.8, and 8.6 Hz, 1H), 6.39 (s, 1H), 5.60 (s, 1H), 3.94 (q, J=7.0 Hz, 2H), 1.36 (t, J=7.0 Hz, 3H), 1.34 (s, 9H); MS (EI) m/z: 413 (M+H⁺).

### Example A21

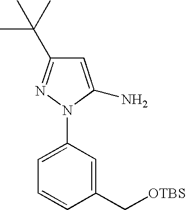

To a solution of CuI (1 mol %), 1,10-phenanthroline (10 mol %), Cs₂CO₃ (9.8 g, 30 mmol) and DMF (20 mL) was added t-butyl carbazate (3.4 g, 25 mmol), 3-iodobenzyl alcohol (5.0 g, 21 mmol). The reaction mixture was heated at 80° C. for 2 h. The reaction mixture was filtered through a pad of silica gel and the filtrate was evaporated under reduced pressure to obtain crude product, 1-Boc-1-(3-carbinol)phenylhydrazine as yellow oil. The product was used for the next reaction without further purification.

To a solution of 1-Boc-1-(3-carbinol)phenylhydrazine (2.0 g, 8.4 mmol) in absolute ethanol (30 mL) at RT was added concentrated HCl (3.5 mL, 42 mmol). The reaction mixture was stirred at 60° C. for 30 min. Pivaloylacetonitrile (1.3 g, 10 mmol) was added into the reaction mixture, which was heated at 90° C. for 3 h. The solvent was evaporated under reduced pressure and the residue was dissolved in water and lyophilized to obtain the crude product [3-(5-amino-3-t-butyl-1H-pyrazol-1-yl)phenyl]methanol as the HCl salt. The product was used for the next step without further purification. ¹H-NMR (DMSO-d₆): δ 7.4-7.6 (m, 4H), 5.62 (br s, 1H), 4.59 (s, 2H), 1.29 (s, 9H).

To a solution of [3-(5-amino-3-t-butyl-1H-pyrazol-1-yl)phenyl]methanol hydrochloride salt (2.0 g, 7.1 mmol) in DMF (20 mL) was added imidazole (2.7 g, 39 mmol) and TBSCl (2.1 g, 14 mmol), which was stirred at RT for 8 h. The reaction mixture was quenched with water and extracted with EtOAc (3×). Organic extracts were washed with NaHCO₃, H₂O and 10% LiCl solution. The combined organic extracts were washed with brine, dried (Na₂SO₄), filtered, concentrated and purified via column chromatography to yield 3-t-butyl-1-(3-[(t-butylmethylsilyloxy)methyl]phenyl}-1H-pyrazol-5-amine in 36% yield (for three steps): ¹H-NMR (CDCl₃): δ 7.3-7.6 (m, 4H), 5.54 (s, 1H), 4.80 (s, 2H), 1.34 (s, 9H), 0.97 (s, 9H), 0.13 (s, 6H); MS (EI) m/z: 360 (M+H⁺).

### Example 406

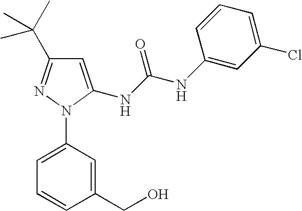

To a solution of Example A21 (100 mg, 0.18 mmol) in THF (2 mL) was added pyridine (45 mL, 0.56 mmol) and 3-chlorophenyl isocyanate (43 mg, 0.18 mmol). The reaction mixture was stirred at RT for 20 min, heated until all solids were dissolved, and stirred at RT for 4 h. The reaction mixture was concentrated under reduced pressure to yield 1-(3-t-butyl-1-{3-[(t-butyldimethylsilyloxy)methyl]phenyl}-1H-pyrazol-5-yl)-3-(3-chlorophenyl)urea (62 mg, 43% yield).

To a solution of 1-(3-t-butyl-1-{3-[(t-butyldimethylsilyloxy)methyl]phenyl}-1H-pyrazol-5-yl)-3-(3-chlorophenyl)urea (120 mg, 0.12 mmol) in THF (2 mL) was added TBAF (1.0 M, 0.13 mL, 0.13 mmol). The reaction mixture was stirred at RT for 2.5 h. The solvent was removed under-reduced pressure. EtOAc was added into the residue followed by 1N-HCl (5 drops). The combined organic extracts were washed with brine, dried (Na₂SO₄), filtered, concentrated and purified via column chromatography to yield 1-(3-t-butyl-1-(3-hydroxymethyl)phenyl)-1H-pyrazol-5-yl)-3-(3-chlorophenyl)urea as a white powder (34 mg, 71% yield). ¹H-NMR (CDCl₃): δ 8.11 (s, 1H), 7.34 (t, J=2.0 Hz, 1H), 7.05-7.25 (m, 7H), 6.99 (dt, J=1.3, and 7.8 Hz, 1H), 6.39 (s, 1H), 4.39 (s, 2H), 1.33 (s, 9H); MS (EI) m/z: 399 (M+H⁺).

### Example 407

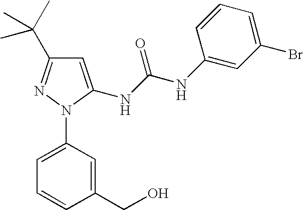

Using the same procedure as for Example 406, Example. A21 (100 mg, 0.28 mmol) and 3-bromophenyl isocyanate (55 mg, 0.28 mmol) were combined to yield 1-{3-t-butyl-1-[3-(hydroxymethyl)phenyl]-1H-pyrazol-5-yl}-3-(3-bromophenyl)urea as a white powder (19 mg, 15% yield). ¹H-NMR (CDCl₃): δ 8.17 (s, 1H), 7.47 (t, J=1.8 Hz, 1H), 7.34 (s, 1H), 7.00-7.25 (m, 7H), 6.39 (s, 1H), 4.37 (s, 2H), 1.32 (s, 9H); MS (EI) m/z: 443 and 445 (M⁺ and M⁺+2H⁺).

### Example 408

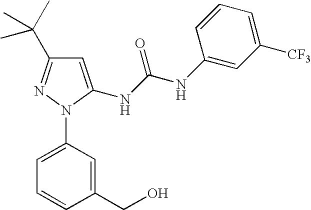

Using the same procedure as for Example 406, Example A20 (100 mg, 0.28 mmol) and 3-(trifluoromethyl)phenyl isocyanate (52 mg, 0.28 mmol) were combined to yield 1-{3-t-butyl-1-[3-(hydroxymethyl)phenyl]-1H-pyrazol-5-yl}-3-(3-(trifluoromethyl)phenyl)urea as a white powder (42 mg, 35% yield). ¹H-NMR (CDCl₃): δ 8.21 (bs, 1H), 7.64 (t, J=1.8 Hz, 1H), 7.1-7.5 (m, 8H), 6.51 (s, 1H), 4.56 (s, 2H), 1.37 (s, 9H); MS (EI) m/z: 433 (M+H⁺).

### Example 409

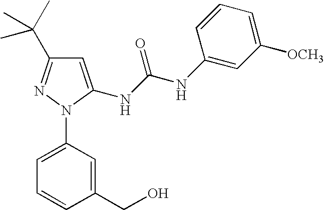

Using the same procedure as for Example 406, Example A21 (100 mg, 0.28 mmol) and 3-methoxyphenyl isocyanate (41 mg, 0.28 mmol) were combined to yield 1-{3-t-butyl-1-[3-(hydroxymethyl)phenyl]-1H-pyrazol-5-yl}-3-(3-methoxyphenyl)urea as a white powder (34 mg, 31% yield). ¹H-NMR (CDCl₃): δ 7.86 (br s, 1H), 7.34 (br s, 1H), 7.31 (d, J=7.6 Hz, 1H), 7.18 (d, J=6.4 Hz, 1H), 7.15 (t, J=8.2 Hz, 1H), 7.00 (t, J=2.1 Hz, 1H), 6.80 (dd, J=1.1, and 8.0 Hz, 1H), 6.62 (dd, J=2.3, and 8.3 Hz, 1H), 6.50 (s, 1H), 4.56 (s, 2H), 3.76 (s, 3H), 1.37 (s, 9H); MS (EI) m/z: 395 (M+H⁺).

### Example 410

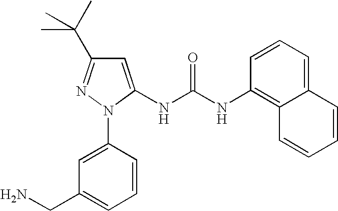

Example A18 was dissolved in dry THF (10 mL) and added to a THF solution (10 mL) of 1-isocyano naphthalene (1.13 g, 6.66 mmol) and pyridine (5.27 g, 66.6 mmol) at RT. The reaction mixture was stirred for 3 h, quenched with H₂O (30 mL), and the resulting precipitate filtered and washed with 1N HCl and ether to yield 1-[2-(3-azidomethyl-phenyl)-5-t-butyl-2H-pyrazol-3-yl]-3-naphthalen-1-yl-urea. (2.4 g, 98%) as a white solid.

The crude material from the previous reaction and Pd/C (0.4 g) in THF (30 mL) was hydrogenated under 1 atm at RT for 2 h. The catalyst was removed by filtration and the filtrate concentrated in vacuo to yield 1-(3-t-butyl-1-(3-(aminomethyl)phenyl)-1H-pyrazol-5-yl)-3-(naphthalen-1-yl)urea (2.2 g, 96%) as a yellow solid. ¹H NMR (DMSO-d₆): 9.02 (s, 1H), 7.91 (d, J=7.2 Hz, 1H), 7.89 (d, J=7.6 Hz, 2H), 7.67-7.33 (m, 9H), 6.40 (s, 1H), 3.81 (s, 2H), 1.27 (s, 9H); MS (ESI) m/z: 414 (M+H⁺).

### Example 411

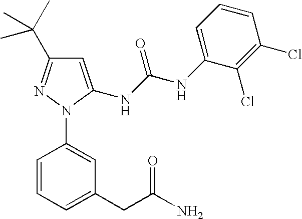

Using the same procedure as for Example 304, Example A10 (136 mg, 0.5 mmol) and added 1,2-dichloro-3-isocyanatobenzene (98 mg, 0.5 mmol) were combined to afford 1-{1-[3-(2-amino-2-oxoethyl)phenyl]-3-t-butyl-1H-pyrazol-5-yl}-3-(2,3-dichlorophenyl)urea (60 mg, 26% yield). ¹H NMR (300 MHz, DMSO-d₆): δ 9.23 (s, 1H), 8.75 (s, 1H), 8.04 (m, 1H), 7.50 (br s, 1H), 7.45-7.25 (m, 7H), 6.90 (br s, 1H), 6.36 (s, 1H), 3.42 (s, 2H), 1.24 (s, 9H); MS (ESI) m/z: 459 (M+H⁺).

### Example 412

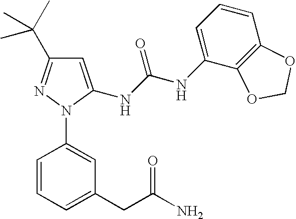

Using the same procedure as for Example 304, Example A10 and the isocyante of benzo[d][1,3]dioxol-4-amine are combined to yield 1-(3-t-butyl-1-(3-(carbamoylmethyl)-phenyl)-1H-pyrazol-5-yl)-3-(benzo[d][1,3]dioxol-7-yl)urea.

### Example 413

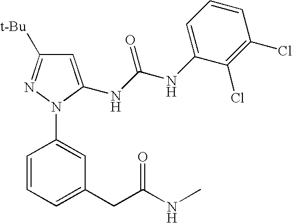

To a solution of Example 351 (100 mg, 0.20 mmol) in anhydrous MeOH (10 mL) was added a solution of CH₃NH₂ (5 mL, 25%) in MeOH at RT. The mixture was heated to 50° C. for 3 h. After removal of the solvent, the residue was purified by preparative HPLC to afford 1-{3-t-butyl-1-[3-(methylamino)-2-oxoethyl]phenyl}-1H-pyrazol-5-yl}-3-(2,3-dichlorophenyl)urea (70 mg, 74% yield). ¹H-NMR (300 MHz, DMSO-d₆): δ 9.40 (br s, 1H), 8.84 (s, 1H), 8.04-8.02 (m, 2H), 7.41-7.33 (m, 3H), 7.27-7.25 (m, 3H), 6.34 (s, 1H), 3.44 (s, 2H), 3.34 (s, 3H), 1.24 (s, 9H); MS (ESI) m/z: 474(M+H⁺).

### Example 414

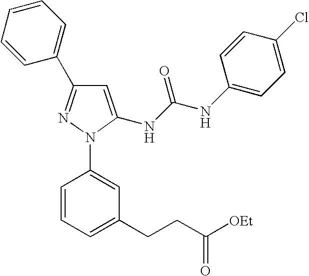

To a solution of commercially available 3-oxo-3-phenyl-propionitrile (1.45 g, 10.0 mmol) and ethanol (690 mg, 15.0 mmol) in CH₂Cl₂ (50 mL) was bubbled HCl gas at 0° C. for 1 h. The resulting mixture was warmed to RT and stirred overnight. After removal of the solvent, the residue was washed with Et₂O to afford 1.6 g of 3-oxo-3-phenyl-propionimidic acid ethyl ester hydrochloride, which was used to the next reaction without further purification. MS (ESI) m/z: 228 (M+H⁺).

To a solution of 3-oxo-3-phenyl-propionimidic acid ethyl ester hydrochloride (1.5 g, 6.6 mmol) and Et₃N (2.02 g, 20 mmol) in THF (50 mL) was added 1-chloro-4-isocyanato-benzene (1.1 g, 7.2 mmol) at 0° C. The resulting mixture was stirred at RT overnight, then poured to water (100 mL). The mixture was extracted with CH₂Cl₂ (3×100 mL). The combined organic extracts were washed with brine, dried (Na₂SO₄), filtered, concentrated and purified via column chromatography to afford 2.0 g of 1-(4-chloro-phenyl)-3-(1-ethoxy-3-oxo-3-phenyl-propenyl)-urea MS (ESI) m/z: 345 (M+H⁺).

A mixture of 3-(3-hydrazino-phenyl)-propionic acid ethyl ester (See Example NN, 500 mg, 2.05 mmol) and 1-(4-chloro-phenyl)-3-(1-ethoxy-3-oxo-3-phenyl-propenyl)-urea (688 mg, 2.0 mol) in ethanol (100 mL) was stirred at RT for 3 h. After removal of the solvent, the residue was purified by column chromatography to yield 700 mg of 3-(3-{5-[3-(4-chloro-phenyl)-ureido]-3-phenyl-pyrazol-1-yl}-phenyl)-propionic acid ethyl ester. ¹H NMR (400 MHz, CD₄O-d₆): 7.83 (d, J=7.6 Hz, 2H), 7.51-7.33 (m, 9H), 7.26 (d, J=8.8 Hz, 2H), 6.89 (s, 1H), 4.09 (q, J=7.2 Hz, 2H), 3.03 (t, J=7.6 Hz, 2H), 2.69 (t, J=7.6 Hz, 2H), 1.20 (t, J=7.2 Hz, 3H). MS (ESI) m/z: 489 (M+H⁺).

### Example 415

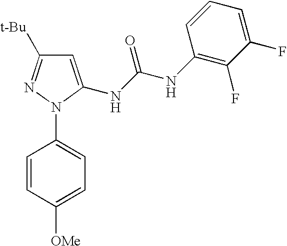

Using the same procedure as for Example 305, Example A8 (123 mg, 0.5 mmol) and 1-fluoro-2,3-difluorophenylamine (65 mg, 0.5 mmol) were combined to afford 1-[3-t-butyl-1-(4-methoxyphenyl)-1H-pyrazol-5-yl]-3-(2,3-difluorophenyl)urea (65 mg, 32% yield). ¹H-NMR (300 MHz, DMSO-d₆): δ 9.08 (s, 1H), 8.77 (s, 1H), 7.90 (t, J=7.2 Hz, 1H), 7.37 (d, J=9.0 Hz, 2H), 7.13-6.95 (m, 4H), 6.33 (s, 1H), 3.79 (s, 3H), 1.23 (s, 9H); MS (ESI) m/z: 401 (M+H⁺).

### Example 416

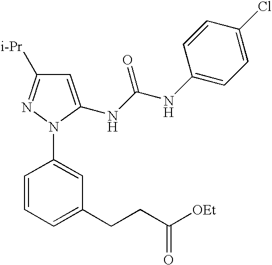

Using the same procedure as for Example 414, 4-methyl-3-oxo-pentanenitrile (from Example A28, 1.11 g, 10.0 mmol) was transformed to 4-methyl-3-oxo-pentanimidic acid ethyl ester hydrochloride (1.0 g, 5.2 mmol), which was combined with 1-chloro-4-isocyanato-benzene (1.1 g, 7.2 mmol) to afford 1.5 g of 1-(4-chlorophenyl)-3-((E)-1-ethoxy-4-methyl-3-oxopent-1-enyl)urea (MS (ESI) m/z: 337 (M+H⁺)). This was combined with 3-(3-hydrazino-phenyl)-propionic acid ethyl ester (from Example A14, 500 mg, 2.05 mmol) to yield 420 mg of ethyl 3-(3-(5-(3-(4-chlorophenyl)ureido)-3-isopropyl-1H-pyrazol-1-yl)phenyl)propanoate. ¹H NMR (400 MHz, CD₄O-d₄): 7.48 (t, J=8.0 Hz, 1H), 7.39-7.35 (m, 5H), 7.25 (d, J=8.8 Hz, 2H), 6.46 (s, 1H), 4.08 (q, J=7.2 Hz, 2H), 3.02-2.98 (m, 3H), 2.67 (t, J=7.6 Hz, 2H), 1.31 (d, J=6.8 Hz, 3H), 1.19 (t, J=7.2 Hz, 3H). MS (ESI) m/z: 455 (M+H⁺).

### Example A22

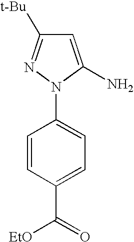

Ethyl 4-(3-t-butyl-5-amino-1H-pyrazol-1-yl)benzoate (3.67 mmol) was prepared from ethyl 4-hydrazinobenzoate and pivaloylacetonitrile by the procedure of Regan, et al., J. Med. Chem., 45, 2994 (2002).

### Example 417

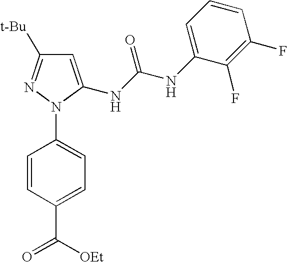

Using the same procedure as for Example 304, Example A22 (287 mg, 1.0 mmol), and 2,3-difluorophenylamine (134 mg, 1.0 mmol) were combined to afford ethyl 4-{3-t-butyl-5-[3-(2,3-difluorophenyl)ureido]-1H-pyrazol-1-yl}benzoate (250 mg, 57% yield).

### Example 418

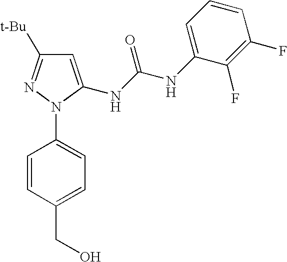

Using the same procedure as for Example 303, Example 417 (230 mg, 0.52 mmol) was reduced to afford 1-{3-t-butyl-1-[4-(hydroxymethyl)phenyl]-1H-pyrazol-5-yl}-3-(2,3-difluoro-phenyl)urea (160 mg, 80% yield). ¹H NMR (300 MHz, DMSO-d₆): δ 9.14 (s, 1H), 8.95 (s, 1H), 7.84-6.82 (m, 7H), 6.25 (s, 1H), 5.27 (t, J=5.7 Hz, 1H), 4.42 (br s, 2H), 1.14 (s, 9H); MS (ESI) m/z: 401 (M+H⁺).

### Example 419

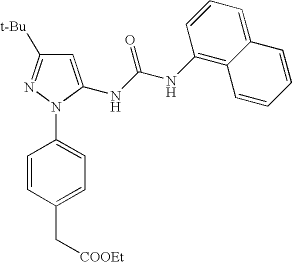

Using the same procedure as for Example 304, Example A1 (5 g, 14.8 mmol) and 1-isocyanatonaphthalene (2.5 g, 15.0 mmol) I were combined to afford ethyl 2-(4-{3-t-butyl-5-[3-(naphthalen-1-yl)ureido]-1H-pyrazol-1-yl}phenyl)acetate (1.7 g, 24% yield). MS (ESI) m/z: 471 (M+H⁺).

### Example 420

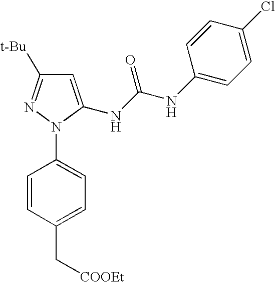

Using the same procedure as for Example 304, Example A1 (5 g, 14.8 mmol) and 1-chloro-4-isocyanato-benzene (2.2 g, 15.0 mmol) were combined to afford ethyl 2-(4-{3-t-butyl-5-[3-(4-chlorophenyl)ureido]-1H-pyrazol-1-yl}phenyl)acetate (2.7 g, 40% yield). ¹H NMR (DMSO-d₆): δ 9.12 (s, 1H), 8.42 (s, 1H), 7.46-7.37 (m, 6H), 7.28 (d, J=8.1 Hz, 2H), 6.34 (s, 1H), 4.08 (q, J=7.2 Hz, 2H), 2.79 (t, J=7.2 Hz, 2H), 3.72 (s, 2H), 1.25 (s, 9H), 1.18 (t, J=7.2 Hz, 3H); MS (ESI) m/z: 455 (M+H⁺).

### Example 421

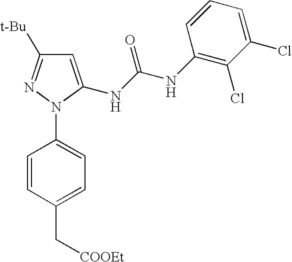

Using the same procedure as for Example 304, Example A1 (5 g, 14.8 mmol) and 1,2-dichloro-3-isocyanatobenzene (2.8 g, 15.0 mmol) were combined to afford 2-(4-{3-t-butyl-5-[3-(2,3-dichlorophenyl)ureido]-1H-pyrazol-1-yl}phenyl)acetic acid (2.1 g, 29% yield). ¹H NMR (DMSO-d₆): δ 9.24 (s, 1H), 8.77 (s, 1H), 8.05 (m, 1H), 7.47-7.38 (m, 4H), 7.30-7.28 (m, 2H), 6.36 (s, 1H), 4.08 (q, J=7.2 Hz, 2H), 2.72 (s, 2H), 1.25 (s, 9H), 1.18 (t, J=7.2 Hz, 3H); MS (ESI) m/z: 489 (M+H⁺).

### Example 422

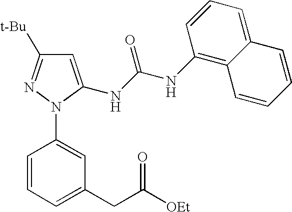

Using the same procedure as for Example 304, Example A9 (5 g, 14.8 mmol) and 1-isocyanatonaphthalene (2.5 g, 15.0 mmol) were combined to afford ethyl 2-(3-{3-t-butyl-5-[3-(naphthalen-1-yl)ureido]-1H-pyrazol-1-yl}phenyl)acetate (1.5 g, 22% yield). MS (ESI) m/z: 471 (M+H⁺).

### Example 423

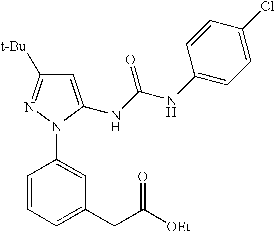

Using the same procedure as for Example 304, Example A9 (5 g, 14.8 mmol) and 1-chloro-4-isocyanato-benzene (2.2 g, 15.0 mmol) were combined to afford ethyl 2-(3-{3-t-butyl-5-[3-(4-chlorophenyl)ureido]-1H-pyrazol-1-yl}phenyl)acetate (2.7 g, 40% yield). ¹H NMR (DMSO-d₆): δ 9.10 (s, 1H), 8.39 (s, 1H), 7.46-7.37 (m, 5H), 7.28-7.25 (m, 3H), 6.34 (s, 1H), 4.04 (q, J=7.2 Hz, 2H), 3.72 (s, 2H), 1.25 (s, 9H), 1.14 (t, J=7.2 Hz, 3H); MS (ESI) m/z: 455 (M+H⁺).

### Example 424

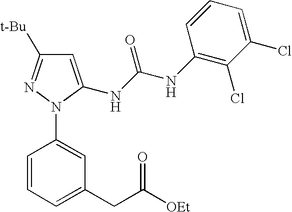

Using the same procedure as for Example 304, Example A9 (5 g, 14.8 mmol) and 1,2-dichloro-3-isocyanato-benzene (2.8 g, 15.0 mmol) were combined to afford ethyl 2-(3-{3-t-butyl-5-[3-(2,3-dichlorophenyl)ureido]-1H-pyrazol-1-yl}phenyl)acetate (2.1 g, 29% yield). ¹H NMR (300 MHz, DMSO-d₆): δ 9.22 (s, 1H), 8.75 (s, 1H), 8.05 (m, 1H), 7.46-7.21 (m, 6H), 6.35 (s, 1H), 4.04 (q, J=7.2 Hz, 2H), 3.72 (s, 2H), 1.24 (s, 9H), 1.16 (t, J=7.2 Hz, 3H); MS (ESI) m/z: 489 (M+H⁺).

### Example A23

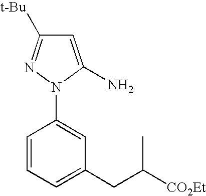

To a suspension of 2-(3-bromo-phenyl)-5-t-butyl-2H-pyrazol-3-ylamine (5.8 g, 20 mmol), Pd(OAc)₂ (450 mg, 2 mmol), PPh₃ (1.0 g, 4 mmol), and K₂CO₃ (5.5 g, 40 mmol) in DMF (50 mL) was added 2-methyl-acrylic acid ethyl ester (2.8 g, 25 mmol) at RT under N₂. The mixture was stirred at 80° C. overnight, concentrated under reduced pressure, and purified by column chromatography to afford (E)-3-[3-(5-amino-3-t-butyl-1H-pyrazol-1-yl)phenyl]-2-methylacrylic acid (3.2 g). MS (ESI) m/z: 328 (M+H⁺)

A mixture of (E)-3-(3-(3-t-butyl-5-amino-1H-pyrazol-1-yl)phenyl)-2-methylacrylic acid ethyl ester (3.0 g, 9.14 mmol) and Pd/C (0.3 g) in methanol (50 mL) was stirred at RT under 40 psi of H₂ for 2 h. The reaction mixture was filtered and the filtrate was concentrated to afford ethyl 3-[3-(5-amino-3-t-butyl-1H-pyrazol-1-yl)phenyl]-2-methylpropanoate (2.5 g, 83% yield). MS (ESI) m/z: 330 (M+H⁺).

### Example 425

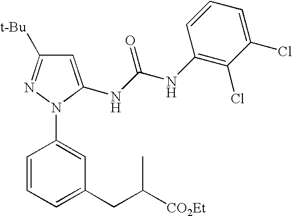

Using the same procedure as for Example 304, Example A23 (200 mg, 0.61 mmol) and 1,2-dichloro-3-isocyanatobenzene (187 mg, 1.0 mmol) were combined to yield 180 ethyl 3-(3-{3-t-butyl-5-[3-(2,3-dichlorophenyl)ureido]-1H-pyrazol-1-yl}phenyl)-2-methylpropanoate (180 mg, 57% yield). MS (ESI) m/z: 517 (M+H⁺).

### Example 426

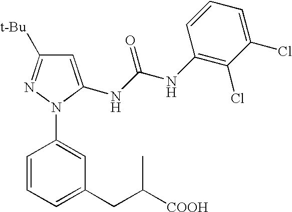

Using the same procedure as for Example 306, Example 425 (100 mg, 0.19 mmol) was saponified to afford 3-(3-{3-t-butyl-5-[3-(2,3-dichlorophenyl)ureido]-1H-pyrazol-1-yl}-phenyl)-2-methylpropanoic acid (60 mg, 65% yield). ¹H-NMR (DMSO-d₆): δ 9.20 (s, 1H), 8.72 (s, 1H), 8.03 (m, 1H), 7.43-7.19 (m, 6H), 6.34 (s, 1H), 2.95 (m, 1H), 2.69-2.62 (m, 2H), 1.24 (s, 9H), 1.01 (d, J=6.3 Hz, 3H); MS (ESI) m/z: 489 (M+H⁺).

### Example A24

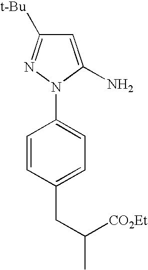

To a mixture of 4-bromo-phenylhydrazine hydrochloride (22.2 g, 0.10 mol) and 4,4-dimethyl-3-oxo-pentanenitrile (13.7 g, 0.11 mol) in ethanol (100 mL) was added conc. HCl (10 mL). The resulting mixture was heated to reflux for 3 h. After removal of the solvent, the residue was purified by column chromatography to yield 2-(4-bromophenyl)-5-t-butyl-2H-pyrazol-3-ylamine hydrochloride (30 g). ¹H-NMR (400 MHz, DMSO-d₆): δ 7.76 (d, J=8.8 Hz, 2H), 7.55 (d, J=8.8 Hz, 2H), 5.63 (s, 1H), 1.27 (s, 9H); MS (ESI) m/z: 294 (M+H⁺).

To a solution of 2-(4-bromophenyl)-5-t-butyl-2H-pyrazol-3-ylamine (3.94 g, 10 mmol.), Pd(OAc)₂ (224 mg, 10% mol.), PPh₃ (520 mg, 20% mol.) and K₂CO₃ (3.28 g, 40 mmol.) in DMF (10 mL) was added 2-methyl-acrylic acid ethyl ester (1.88 mL, 15 mmol.) under N₂. The resulting mixture was stirred at 90° C. for 12 h. After removal of the solvent, the residue was extracted with EtOAc (3×150 mL). The combined organic extracts were washed with brine, dried (Na₂SO₄), filtered, concentrated and purified via column chromatography to yieldo of ethyl 3-[4-(5-amino-3-t-butyl-pyrazol-1-yl)-phenyl]-2-methyl-acrylate (900 mg).

A mixture of ethyl 3-[4-(5-amino-3-t-butyl-pyrazol-1-yl)-phenyl]-2-methyl-acrylate (900 mg, 2.7 mmol) and Pd/C (0.1 g) in EtOH (20 mL) was stirred at RT under 40 psi of H₂ for 2 h, and then filtered through celite. The filtrate was concentrated to afford ethyl 3-[4-(5-amino-3-t-butyl-1H-pyrazol-1-yl)phenyl]-2-methylpropanoate (850 mg), which was used for the next reaction without further purification.

### Example 427

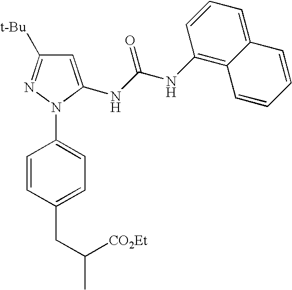

Using the same procedure as for Example 304, Example A24 (100 mg, 0.30 mmol) and 1-naphthyl isocyanate (70 mg, 0.41 mmol) were combined to afford ethyl 3-(4-{3-t-butyl-5-[3-(naphthalen-1-yl)ureido]-1H-pyrazol-1-yl}phenyl)-2-methylpropanoate (75 mg, 50% yield). ¹H-NMR (CD₃OD): δ 7.87 (m, 2H), 7.69 (m, 2H), 7.43-7.51 (m, 5H), 7.38 (d, J=8.0 Hz, 2H), 6.43 (s, 1H), 4.08 (m, 2H), 3.05 (m, 1H), 2.80 (m, 2H), 1.33 (s, 9H), 1.18 (d, J=8.0 Hz, 3H), 1.19 (t, J=8.0 Hz, 3H); MS (ESI) m/z: 499 (M+H⁺).

### Example 428

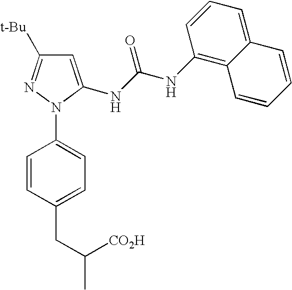

Using the same procedure as for Example 306, Example 428 (30 mg, 0.06 mmol) was saponified to afford 3-(4-{3-t-butyl-5-[3-(naphthalen-1-yl)ureido]-1H-pyrazol-1-yl}phenyl)-2-methylpropanoic acid (15 mg, 53% yield). ¹H NMR (DMSO-d₆): δ 9.15 (s, 1H), 8.95 (s, 1H), 8.05 (d, J=7.2 Hz, 1H), 7.89 (d, J=7.2 Hz, 1H), 7.61(d, J=8.0 Hz, 1H), 7.41-7.55 (m, 5H), 7.32-7.34 (d, J=8.0 Hz, 2H), 6.36 (s, 1H), 2.96 (m, 1H), 2.66 (m, 2H), 1.23 (s, 9H), 1.06 (d, J=6.4 Hz, 3H); MS (ESI) m/z: 471 (M+H⁺).

### Example 429

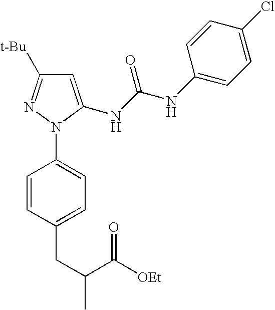

Using the same procedure as for Example 304, Example A24 (100 mg, 0.30 mmol) and 1-chloro-4-isocyanato-benzene (69 mg, 0.45 mmol) were combined to afford ethyl 3-(4-{3-t-butyl-5-[3-(4-chlorophenyl)ureido]-1H-pyrazol-1-yl}phenyl)-2-methylpropanoate (90 mg, 62% yield). ¹H-NMR (400 MHz, CD₃OD): δ 7.34-7.41 (m, 6H), 7.25 (d, J=8.8 Hz, 2H), 6.40 (s, 1H), 4.05-4.08 (m, 2H), 3.03 (m, 1H), 2.80 (m, 2H), 1.28 (s, 9H), 1.19 (t, J=8.0 Hz, 3H), 1.17 (d, J=6.4 Hz, 3H); MS (ESI) m/z: 483 (M+H⁺).

### Example 430

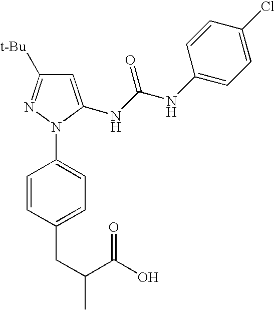

Using the same procedure as for Example 306, Example 429 (40 mg, 0.08 mmol) was saponified to afford 3-(4-{3-t-butyl-5-[3-(4-chlorophenyl)ureido]-1H-pyrazol-1-yl}phenyl)-2-methylpropanoic acid (18 mg, 50% yield). ¹H-NMR (400 MHz, DMSO-d₆): δ 7.37-7.43 (m, 4H), 7.24-7.30 (m, 4H), 6.28 (s, 1H), 2.95 (m, 2H), 2.64 (m, 1H), 1.24 (s, 9H), 1.15 (d, J=7.6 Hz, 3H). MS (ESI) m/e: 455 (M+H⁺).

### Example A26

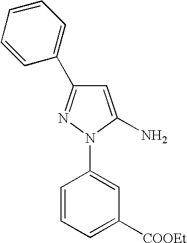

To a solution of m-amino benzoic acid ethyl ester (200 g, 1.46 mmol) in concentrated HCl (200 mL) was added an aqueous solution (250 mL) of NaNO₂ (102 g, 1.46 mmol) at 0° C. and the reaction mixture was stirred for 1 h. A solution of SnCl₂.2H₂O (662 g, 2.92 mmol) in concentrated HCl (2 L) was then added at 0° C. The reaction solution was stirred for 2 h at RT. The precipitate was filtered and washed with ethanol and ether to yield ethyl 3-hydrazinobenzoate, which was used for the next reaction without further purification.

To a mixture of 3-hydrazino-benzoic acid ethyl ester (4.5 g, 25.0 mmol) and commercially available 3-oxo-3-phenyl-propionitrile (5.5 g, 37.5 mmol) in ethanol (50 mL) was added conc. HCl (5 mL). The resulting mixture was heated to reflux for 3 h. After removal of the solvent, the residue was washed with Et₂O to afford ethyl 3-(5-amino-3-phenyl-1H-pyrazol-1-yl)benzoate (7 g), which was used in the next reaction without further purification.

### Example 431

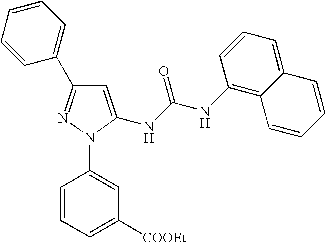

Using the same procedure as for Example 304, Example A26 (1.54 g, 5.0 mmol) and 1-isocyanato-naphthalene (1.0 g, 6.0 mmol) were combined to afford ethyl 3-[5-(3-naphthalen-1-yl-ureido)-3-phenyl-pyrazol-1-yl]benzoate (970 mg, 41% yield).

### Example 432

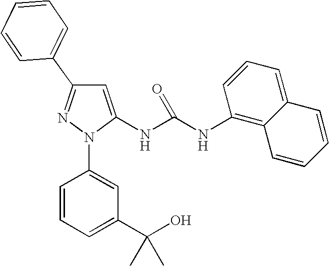

To a solution of Example 431 (100 mg, 0.21 mmol) in fresh THF (10 mL) was added dropwise a solution of MeMgBr (0.7 mmol, 1M in THF) at 0° C. in ice-water bath. The resulting mixture was stirred for 1 h, and then warmed to RT for 2 h. The reaction mixture was quenched with saturated NH₄Cl (10 mL) and extracted with CH₂Cl₂ (3×50 mL). The combined organic extracts were washed with saturated NaHCO₃ and brine, then dried (Na₂SO₄), filtered, concentrated and purified via preparative HPLC to afford 1-{1-[3-(2-hydroxypropan-2-yl)phenyl]-3-phenyl-1H-pyrazol-5-yl}-3-(naphthalen-1-yl)urea (85 mg, 88% yield). ¹H-NMR (300 MHz, CD₃OD): δ 7.85-7.82 (m, 4H), 7.77 (m, 1H), 7.73-7.62 (m, 3H), 7.56 (m, 1H), 7.50-7.39 (m, 6H), 7.35 (m, 1H), 6.91 (s, 1H), 1.58 (s, 6H).

### Example A27

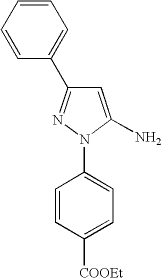

A solution of 4-aminobenzoic acid ethyl ester (200 g, 1.46 mmol) in concentrated HCl (200 mL) was added an aqueous solution (250 mL) of NaNO₂ (102 g, 1.46 mmol) at 0° C. and the reaction mixture was stirred for 1 h. A solution of SnCl₂.2H₂O (662 g, 2.92 mmol) in concentrated HCl (2L) was then added at 0° C. The reaction solution was stirred for 2 h at RT. The precipitate was filtered and washed with ethanol and ether to yield 4-hydrazinobenzoic acid ethyl ester, which was used in the next reaction without further purification.

To a mixture of 4-hydrazinobenzoic acid ethyl ester (4.5 g, 25 mmol) and commercially available 3-oxo-3-phenylpropionitrile (5.5 g, 37.5 mmol) in ethanol (50 mL) was added conc. HCl (5 mL). The resulting mixture was heated to reflux for 3 h. After removal of the solvent, the residue was washed with Et₂O to afford ethyl 4-(5-amino-3-phenyl-1H-pyrazol-1-yl)benzoate (7.4 g), which was used in the next reaction without further purification.

### Example 433

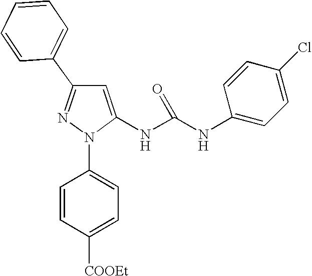

Using the same procedure as for Example 304, Example A27 (1.54 g, 5.0 mmol) and 1-chloro-4-isocyanatobenzene (0.92 g, 6.0 mol) were transformed to afford ethyl 4-{5-[3-(4-chlorophenyl)ureido]-3-phenyl-1H-pyrazol-1-yl}benzoate (1.2 g, 52% yield).

### Example 434

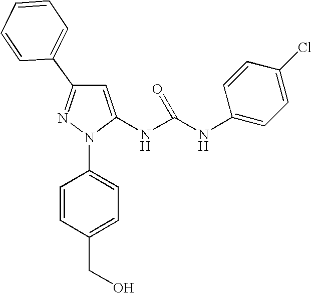

Using the same procedure as for Example 303, Example 433 (100 mg, 0.21 mmol) was reduced to afford 1-(4-chlorophenyl)-3-{1-[4-(hydroxymethyl)phenyl]-3-phenyl-1H-pyrazol-5-yl}-urea (70 mg, 80% yield). ¹H-NMR (300 MHz, CD₃OD): δ 9.16 (s, 1H), 8.53 (s, 1H), 7.81 (d, J=7.2 Hz, 2H), 7.54 (d, J=8.4 Hz, 2H), 7.49-7.40 (m, 4H), 7.38-7.28 (m, 3H), 6.89 (s, 1H), 5.30 (t, J=5.6 Hz, 1H), 4.56 (d, J=5.6 Hz, 2H).

### Example A28

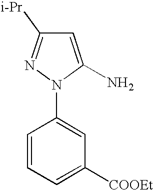

To a suspension of NaH (60%, 6.0 g, 0.15 mol) in THF (100 mL) was added dropwise isobutyric acid ethyl ester (11.6 g, 0.1 mol) and anhydrous acetonitrile (50 g, 0.12 mol) in THF (100 mL) at 80° C. The resulting mixture was refluxed overnight, then cooled to RT. After removal of the volatiles in vacuo, the residue was diluted in EtOAc and aqueous 10% HCL. The combined organic extracts were dried (Na₂SO₄), filtered, concentrated to yield 4-methyl-3-oxopentanenitrile (8.5 g), which was used for the next step reaction without further purification.

To a mixture of ethyl 3-hydrazino-benzoate (from Example OO, 3 g, 16.6 mmol) and 4-methyl-3-oxopentanenitrile (2.7 g, 24.9 mmol) in ethanol (50 mL) was added conc. HCl (5 mL). The resulting mixture was heated to reflux for 3 h. After removal of the solvent, the residue was washed with Et₂O to afford ethyl 3-(5-amino-3-isopropyl-1H-pyrazol-1-yl)-benzoate (4 g), which was used in the next reaction without further purification.

### Example 435

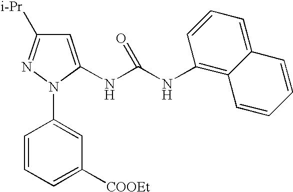

Using the same procedure as for Example 304, Example A28 (1.37 g, 5.0 mmol) and 1-isocyanato-naphthalene (1.0 g, 60 mol) were combined to afford ethyl 3-{3-isopropyl-5-[3-(naphthalen-1-yl)ureido]-1H-pyrazol-1-yl}benzoate (1.02 g, 46% yield).

### Example 436

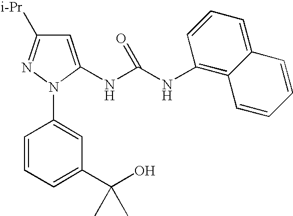

Using the same procedure as for Example 432, Example 435 (100 mg, 0.23 mmol) was transformed to afford 1-{1-[3-(2-hydroxypropan-2-yl)phenyl]-3-isopropyl-1H-pyrazol-5-yl}-3-(naphthalen-1-yl)urea (80 mg, 81% yield). ¹H-NMR (300 MHz, DMSO-d₆): δ 9.00 (s, 1H), 8.78 (s, 1H), 7.95 (m, 1H), 7.90-7.87 (m, 2H), 7.63-7.60 (m, 2H), 7.54-7.34 (m, 6H), 6.33 (s, 1H), 2.88 (m, 1H), 1.43 (s, 6H), 1.21 (d, J=6.9 Hz, 3H).

### Example A29

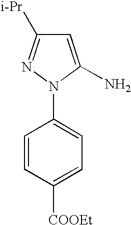

To a mixture of 4-hydrazino-benzoic acid ethyl ester (from Example PP, 3 g, 16.6 mmol) and 4-methyl-3-oxo-pentanenitrile (from Example QQ, 2.7 g, 27.9 mmol) in ethanol (50 mL) was added conc. HCl (5 mL). The resulting mixture was heated to reflux for 3 h. After removal of the solvent, the residue was washed with Et₂O to afford ethyl 4-(5-amino-3-isopropyl-1H-pyrazol-1-yl)benzoate (4 g, 88% yield), which was used to the next reaction without further purification.

### Example 437

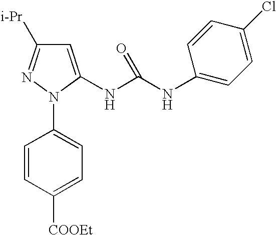

Using the same procedure as for Example 304, Example A29 (1.37 g, 5.0 mmol) and 1-chloro-4-isocyanatobenzene (0.9 g, 60 mol) were combined to afford ethyl 4-{5-[3-(4-chlorophenyl)ureido]-3-isopropyl-1H-pyrazol-1-yl}benzoate (1.3 g, 61% yield).

### Example 438

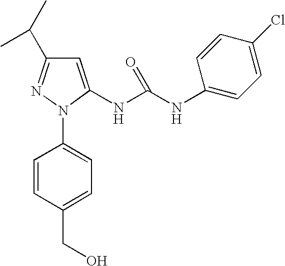

Using the same procedure as for Example 303, Example 437 (100 mg, 0.23 mmol) was reduced to afford 1-(4-chlorophenyl)-3-{1-[4-(hydroxymethyl)phenyl]-3-isopropyl-1H-pyrazol-5-yl}-urea (80 mg, 91% yield). ¹H-NMR (400 MHz, DMSO-d₆): δ 9.15 (br s, 1H), 8.70 (br s, 1H), 7.46-7.36 (m, 6H), 7.26 (d, J=8.8 Hz, 2H), 6.25 (s, 1H), 5.28 (t, J=6.0 Hz, 1H), 4.52 (d, J=5.2 Hz, 2H), 2.85 (m, 1H), 1.20 (d, J=6.8 Hz, 6H).

### Example A30

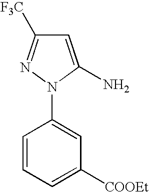

To a mixture of 3-hydrazino-benzoic acid ethyl ester (from Example A26, 3 g, 16.6 mmol) and commercially available 4,4,4-trifluoro-3-oxo-butyronitrile (3.4 g, 24.9 mmol) in ethanol (50 mL) was added conc. HCl (5 mL). The resulting mixture was heated to reflux for 3 h. After removal of the solvent, the residue was washed with Et₂O to afford ethyl 3-[5-amino-3-(trifluoromethyl)-1H-pyrazol-1-yl]benzoate (4.5 g, 91% yield), which was used to the next reaction without further purification.

### Example 439

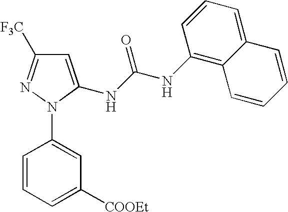

Using the same procedure as for Example 304, Example A30 (1.5 g, 5.0 mmol) and 1-isocyanato-naphthalene (1.0 g, 6.0 mmol) were combined to afford ethyl 3-{5-[3-(naphthalen-1-yl)ureido]-(3-(trifluoromethyl)-1H-pyrazol-1-yl}benzoate (0.9 g, 38% yield).

### Example 440

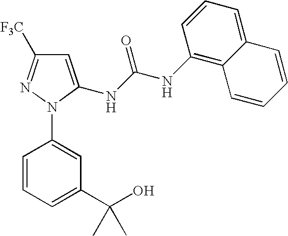

Using the same procedure as for Example 432, Example 439 (100 mg, 021 mmol) was reduced to afford 1-{1-[3-(2-hydroxypropan-2-yl)phenyl]-(3-(trifluoromethyl)-1H-pyrazol-5-yl}-3-(naphthalen-1-yl)urea (50 mg, 52% yield). ¹H-NMR (300 MHz, DMSO-d₆): δ 9.13 (s, 1H), 9.09 (s, 1H), 7.97-7.87 (m, 3H), 7.69-7.63 (m, 4H), 7.58-7.43 (m, 5H), 6.89 (s, 1H), 1.46 (s, 6H).

### Example A31

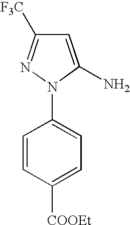

To a mixture of 4-hydrazino-benzoic acid ethyl ester (From Example PP, 3.0 g, 16.6 mmol) and commercially available 4,4,4-trifluoro-3-oxobutyronitrile (3.4 g, 24.9 mmol) in ethanol (50 mL) was added conc. HCl (5 mL). The resulting mixture was heated to reflux for 3 h. After removal of the solvent, the residue was washed with Et₂O to afford ethyl 4-[5-amino-3-(trifluoromethyl)-1H-pyrazol-1-yl]benzoate (4.5 g, 91% yield), which was used to the next reaction without further purification.

### Example 441

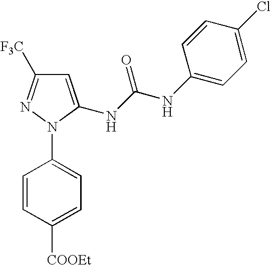

Using the same procedure as for Example 304, Example A31 (1.45 g, 5.0 mmol) and 1-chloro-4-isocyanatobenzene (0.9 g, 6.0 mol) were combined to afford ethyl 4-{5-[3-(4-chlorophenyl)ureido]-3-(trifluoromethyl)-1H-pyrazol-1-yl}benzoate (0.85 g, 38% yield).

### Example 442

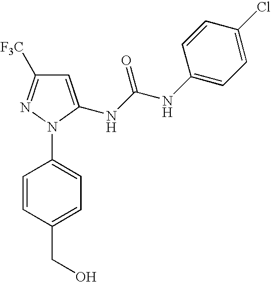

Using the same procedure as for Example 303, Example 441 (100 mg, 0.22 mmol) was reduced to afford 1-(4-chlorophenyl)-3-{3-(trifluoromethyl)-1-[4-(hydroxyl-methyl)phenyl]-1H-pyrazol-5-yl}urea (80 mg, 89% yield). ¹H-NMR (400 MHz, DMSO-d₆): δ 9.65 (s, 1H), 9.09 (s, 1H), 7.54 (d, J=8.4 Hz, 2H), 7.48 (d, J=8.4 Hz, 2H), 7.41 (d, J=8.8 Hz, 2H), 7.28 (d, J=8.8 Hz, 2H), 6.81 (s, 1H), 5.36 (t, J=6.0 Hz, 1H), 4.56 (d, J=5.6 Hz, 2H).

### Example A32

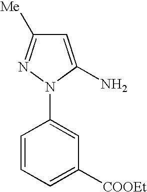

To a suspension of NaH (60%, 12.0 g, 0.3 mol) in THF (200 mL) was added dropwise acetic acid ethyl ester (17 g, 0.2 mol) and anhydrous acetonitrile (100 g, 0.24 mol) in THF (20mL) at 80° C. The resulting mixture was refluxed overnight, and then cooled to RT. After removal of the volatiles in vacuo, the residue was diluted in EtOAc and aqueous 10% HCL. The combined organic extracts were washed with saturated NaHCO₃ and brine, then dried (MgSO₄), filtered, concentrated to yield 3-oxobutyronitrile (10 g), which was used for the next step reaction without further purification.

To a mixture of 3-hydrazino-benzoic acid ethyl ester (from Example OO, 3.0 g, 16.6 mmol) and 3-oxo-butyronitrile (2.1 g, 24.9 mmol) in ethanol (50 mL) was added conc. HCl (5 mL). The resulting mixture was heated to reflux for 3 h. After removal of the solvent, the residue was washed with Et₂O to afford ethyl 3-(5-amino-3-methyl-1H-pyrazol-1-yl)benzoate (4 g), which was used to the next reaction without further purification.

### Example 443

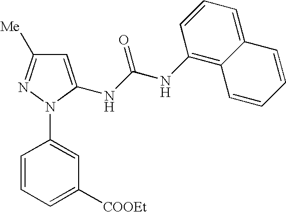

Using the same procedure as for Example 304, Example A32 (490 mg, 2.0 mmol) and 1-isocyanato-naphthalene (0.5 g, 3.0 mmol) were combined to afford ethyl 3-{3-methyl-5-[3-(naphthalen-1-yl)ureido]-1H-pyrazol-1-yl}benzoate (400 mg, 48% yield).

### Example 444

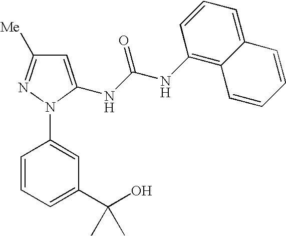

Using the same procedure as for Example 432, Example 443 (100 mg, 0.24 mmol) was reduced to afford 1-{1-[3-(2-hydroxypropan-2-yl)phenyl]-3-methyl-1H-pyrazol-5-yl}-3-(naphthalen-1-yl)urea (80 mg, 83% yield). ¹H-NMR (300 MHz, CDCCl₃): δ 8.61 (br s, 1H), 8.34 (br s, 1H), 7.81-7.78 (m, 2H), 7.67-7.61 (m, 3H), 7.45-7.35 (m, 4H), 7.22 (m, 1H), 7.06 (m, 1H), 6.59 (s, 1H), 2.67 (s, 3H), 1.45 (s, 6H).

### Example 445

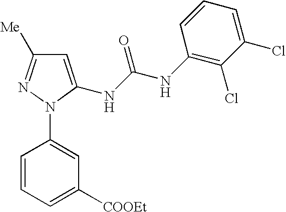

Using the same procedure as for Example 304, Example A32 (490 g, 2.0 mmol) and 1,2-dichloro-3-isocyanatobenzene (448 mg, 3.0 mmol) were combined to afford ethyl 3-{5-[3-(2,3-dichlorophenyl)ureido]-3-methyl-1H-pyrazol-1-yl}benzoate (310 mg, 36% yield).

### Example 446

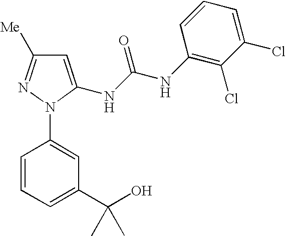

Using the same procedure as for Example 432, Example 445 (100 mg, 0.23 mmol) was reduced to afford 1-(2,3-dichlorophenyl)-3-{1-[3-(2-hydroxypropan-2-yl)phenyl]-3-methyl-1H-pyrazol-5-yl}urea (90 mg, 93% yield). ¹H-NMR (300 MHz, CDCl₃): δ 8.15 (br s, 1H), 8.06 (m, 1H), 7.95 (s, 1H), 7.69 (s, 1H), 7.42 (d, J=5.7 Hz, 2H), 7.30 (m, 1H), 7.19-7.17 (m, 2H), 6.51 (s, 1H), 2.36 (s, 3H), 1.56 (s, 6H).

### Example A33

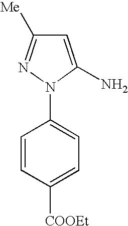

To a mixture of 4-hydrazinobenzoic acid ethyl ester (3.0 g, 16.6 mmol) and 3-oxo-butyronitrile (2.1 g, 25 mmol) in ethanol (50 mL) was added conc. HCl (5 mL). The resulting mixture was heated to reflux for 3 h. After removal of the solvent, the residue was washed with Et₂O to afford ethyl 4-(5-amino-3-methyl-1H-pyrazol-1-yl)benzoate (4 g, 98% yield), which was used to the next reaction without further purification.

### Example 447

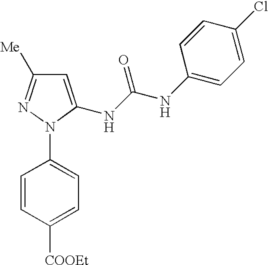

Using the same procedure as for Example 304, Example A33 (1.25 g, 5.0 mmol) and 1-chloro-4-isocyanatobenzene (0.9 g, 6.0 mmol) were combined to afford ethyl 4-{5-[3-(4-chlorophenyl)ureido]-3-methyl-1H-pyrazol-1-yl}benzoate (1.2 g, 60% yield).

### Example 448

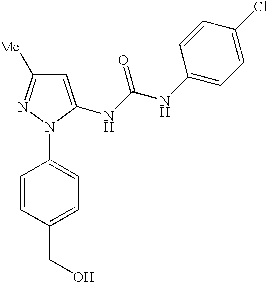

Using the same procedure as for Example 303, Example 447 (100 mg, 0.25 mmol) was reduced to afford 1-(4-chlorophenyl)-3-{1-[4-(hydroxymethyl)phenyl]-3-methyl-1H-pyrazol-5-yl}urea (85 mg, 96% yield). ¹H-NMR (300 MHz, DMSO-d₆): δ 9.83 (s, 1H), 8.90 (s, 1H), 7.47-7.37 (m, 6H), 7.28-7.25 (m, 2H), 6.19 (s, 1H), 5.31 (t, J=6.0 Hz, 1H), 4.51 (d, J=5.7 Hz, 2H), 2.16 (s, 3H).

### Example 449

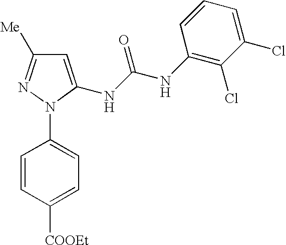

Using the same procedure as for Example 304, Example A33 (1.25 g, 5.0 mmol) and 1,2-dichloro-3-isocyanatobenzene (1.12 g, 6.0 mol) were combined to afford 870 mg of ethyl 4-{5-[3-(2,3-dichlorophenyl)ureido]-3-methyl-1H-pyrazol-1-yl}benzoate (870 mg, 40% yield).

### Example 450

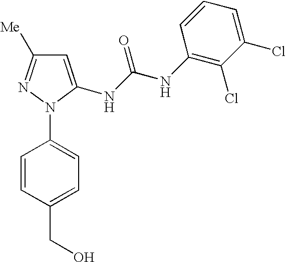

Using the same procedure as for Example 303, Example 449 (100 mg, 0.23 mmol) was reduced to afford 76 mg of 1-(2,3-dichlorophenyl)-3-{1-[4-(hydroxymethyl)phenyl]-3-methyl-1H pyrazol-5-yl)urea (76 mg, 85% yield). ¹H-NMR (300 MHz, CD₃OD): δ 9.59 (s, 1H), 8.95 (s, 1H), 7.99 (m, 1H), 7.48-7.40 (m, 4H), 7.28-7.26 (m, 2H), 6.22 (s, 1H), 5.35 (m, 1H), 4.52 (d, J=4.8 Hz, 2H), 2.17 (s, 3H).

### Example A34

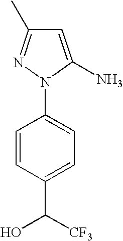

To a solution of 4-nitrobenzaldehyde (15.1 g, 0.1 mol) in THF (100 mL) was added trimethyltrifluoromethylsilane (21.3 g, 0.15 mol) and Bu₄NF (500 mg) at 0° C. under N₂ atmosphere. The resulting mixture was stirred at 0° C. for 1 h and was then warmed to RT. After stirring at RT for 2 h, the reaction mixture was treated of 3.0 N HCl (100 mL). The mixture was then stirred for 1 h, then extracted with CH₂Cl₂ (3×150 mL). The combined organic extracts were washed with saturated NaHCO₃ and brine, then dried (Na₂SO₄), filtered, concentrated and purified via by column chromatography to afford of the desired product 1-(4-nitrophenyl)-2,2,2-trifluoroethanol (17.2 g). ¹H NMR (DMSO-d₆): δ 8.25 (d, J=8.8 Hz, 2H), 7.76 (d, J=8.4 Hz, 2H), 7.15 (d, J=5.6 Hz, 1H), 5.41 (m, 1H).

To a solution of 1-(4-nitrophenyl)-2,2,2-trifluoroethanol (16.0 g, 72 mmol) in methanol (50 mL) was added Pd/C (1.6 g). The mixture was stirred at RT under H₂ at 40 psi for 2 h. After filtration through celite, the filtrate was concentrated to afford 1-(4-aminophenyl)-2,2,2-trifluoroethanol (12 g), which was used for the next reaction without further purification. MS (ESI) m/z: 192 (M+H⁺).

To a stirring solution of 1-(4-aminophenyl)-2,2,2-trifluoroethanol (12 g, 63 mmol) in conc. HCl (80 mL) was added dropwise aqueous NaNO₂ (4.5 g, 65 mmol) at 0° C., and stirred for 1 h. A solution of SnCl₂ (29.5 g, 0.13 mol) in conc. HCl (100 mL) was then added dropwise to the mixture, which was stirred 0° C. for 2 h, then quench with water and neutralized to pH=8. The reaction mixture was extracted with CH₂Cl₂ (3×150 mL). The combined organic extracts were washed with saturated NaHCO₃ and brine, then dried (Na₂SO₄), filtered, concentrated to yield 1-(4-hydrazinophenyl)-2,2,2-trifluoroethanol (10 g), which was used for the next reaction without further purification. MS (ESI) m/z: 207 (M+H⁺).

To a solution of 1-(4-hydrazinophenyl)-2,2,2-trifluoroethanol (1.0 g, 41 mmol) and 3-oxobutyronitrile (500 mg) in ethanol (50 mL) was added 5 mL of conc. HCl. The resulting mixture was heated to reflux for 3 h. After removal of the solvent, the residue was purified by column chromatography to afford 1-[(4-(5-amino-3-methyl-1H-pyrazol-1-yl)phenyl]-2,2,2-trifluoroethanol (1.1 g). MS (ESI) m/z: 272 (M+H⁺).

### Example 451

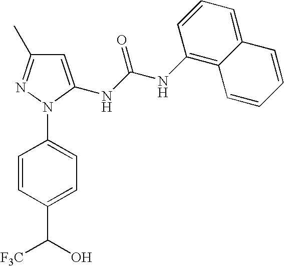

Using the same procedure as for Example 304, Example A34 (500 mg, 1.8 mmol) and 1-isocyanatonaphthalene (338 mg, 2.0 mol) were combined to afford 1-{3-methyl-1-[4-(2,2,2-trifluoro-1-hydroxyethyl)phenyl]-1H-pyrazol-5-yl}-3-(naphthalen-1-yl)urea (100 mg, 13% yield). ¹H NMR (DMSO-d₆): δ 9.01 (s, 1H), 8.85 (s, 1H), 7.97 (d, J=7.2 Hz, 1H), 7.91-7.85 (m, 2H), 7.64-7.40 (m, 8H), 6.30 (s, 1H), 5.24 (m, 1H), 2.17 (s, 3H); MS (ESI) m/z: 441(M+H⁺).

### Example 452

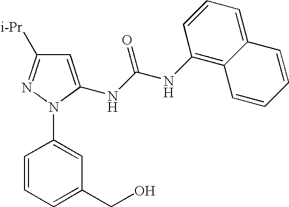

Using the same procedure as for Example 303, Example 435 (1.5 g, 3.4 mmol) was reduced to afford 1-{1-[3-(hydroxymethyl)phenyl]-3-isopropyl-1H-pyrazol-5-yl}-3-(naphthalen-1-yl)-urea (1.2 g, 88% yield), which was used for the next reaction without further purifications. MS (ESI) m/z: 401 (M+H⁺).

### Example 453

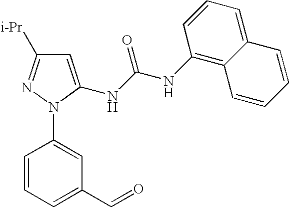

To a solution of Example 452 (1.0 g, 2.5 mmol) in CH₂Cl₂ (50 mL) was added MnO₂ (1.0 g) at RT. The mixture was stirred overnight then filtered. The filtrate was concentrated to afford 1-[1-(3-formylphenyl)-3-isopropyl-1H-pyrazol-5-yl]-3-(naphthalen-1-yl)urea (700 mg, 70% yield), which was used for the next reaction without further purifications. MS (ESI) m/z: 399 (M+H⁺).

### Example 454

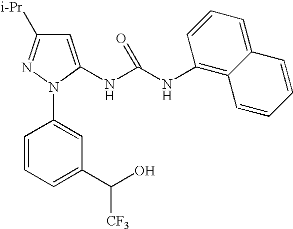

To a solution of Example 453 (500 mg, 1.25 mmol) in THF (50 mL) was added trimethyltrifluoromethylsilane (213 mg, 1.5 mmol) and TBAF (20 mg) at 0° C. The mixture was stirred at RT overnight before quenched with 2.0 N HCl (150 mL). The mixture was then extracted with CH₂Cl₂ (3×150 mL). The combined organic extracts were washed with saturated NaHCO₃ and brine, then dried (Na₂SO₄), filtered, concentrated purified via preparative HPLC to afford 1-{3-isopropyl-1-[3-(2,2,2-trifluoro-1-hydroxyethyl)phenyl]-1H-pyrazol-5-yl}-3-(naphthalen-1-yl)urea (80 mg, 14% yield). ¹H NMR (DMSO-d₆): δ 9.02 (s, 1H), 8.84 (s, 1H), 7.98 (d, J=7.2 Hz, 1H), 7.90-7.85 (m, 2H), 7.64-7.40 (m, 8H), 6.35 (s, 1H), 5.24 (m, 1H), 2.84 (m, 1H), 1.22 (s, 3H), 1.19 (s, 3H); MS (ESI) m/z 469 (M+H⁺).

### Example A35

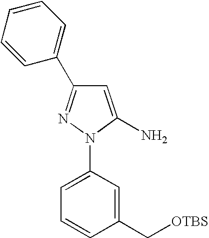

Using the same procedure as for Example KK, benzoylacetonitrile (300 mg, 2.1 mmol) and 1-Boc-1-(3-carbinol)phenylhydrazine (From Example KK, 500 mg, 2.1 mmol) were combined, and then protected with TBSCl as described to afford 1-{3-[(t-butyldimethylsilyloxy)methyl]phenyl}-3-phenyl-1H-pyrazol-5-amine as a brown oil (650 mg, 82% yield). MS (ESI) m/z: 380 (M+H⁺).

### Example 455

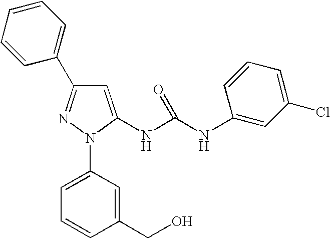

Using the same procedure as for Example 406, Example A35 (120 mg, 0.32 mmol) and 3-chlorophenyl isocyanate (49 mg, 0.32 mmol) were combined to yield 1-{3-phenyl-1-[3-(hydroxymethyl)phenyl]-1H-pyrazol-5-yl}-3-(3-chlorophenyl)urea as a white powder (19 mg, 47% yield). ¹H-NMR (DMSO-d₆): δ 9.32 (s, 1H), 8.66 (s, 1H), 7.86 (m, 1H), 7.70 (t, J=1.6 Hz, 1H), 7.58 (br s, 1H), 7.2-7.55 (m, 7H), 7.04 (m, 1H), 6.95 (s, 1H), 4.51 (s, 2H); MS (EI) m/z: 419 (M+H⁺).

### Example 456

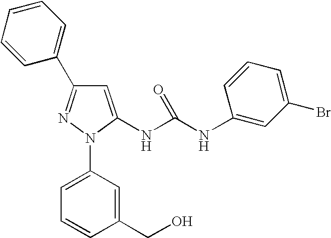

Using the same procedure as for Example 406, Example A35 (120 mg, 0.32 mmol) and 3-bromophenyl isocyanate (63 mg, 0.32 mmol) were combined to yield 1-(3-bromophenyl)-3-{1-[3-(hydroxymethyl)phenyl]-3-phenyl-1H-pyrazol-5-yl}urea as a white powder (33 mg, 75% yield). ¹H-NMR (DMSO-d₆): δ 9.26 (s, 1H), 8.63 (s, 1H), 7.86 (m, 2H), 7.57 (s, 1H), 7.2-7.55 (m, 6H), 7.17 (dt, J=1.8, and 7.4 Hz, 1H), 6.94 (s, 1H), 5.19 (br s, 1H), 4.61 (s, 2H); MS (EI) m/z: 463 and 465 (M⁺ and M+2H⁺).

### Example 457

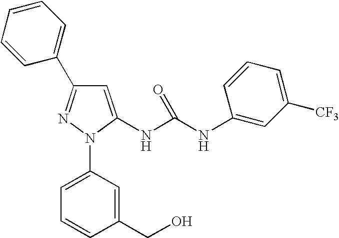

Using the same procedure as for Example 406, Example A35 (120 mg, 0.32 mmol) and 3-(trifluoromethyl)phenyl isocyanate (59 mg, 0.32 mmol) were combined to yield 1-{3-phenyl-1-[3-(hydroxymethyl)phenyl]-1H-pyrazol-5-yl}-3-(3-trifluoromethylphenyl)urea as a white powder (30 mg, 77% yield). ¹H-NMR (DMSO-d₆): δ 9.47 (br s, 1H), 9.03 (br s, 1H), 8.00 (s, 1H), 7.86 (d, J=8.4 Hz, 2H), 7.64 (s, 1H), 7.1-7.6 (m, 9H), 6.92 (s, 1H), 5.49 (t, J=5.6 Hz, 1H), 4.59 (d, J=5.6 Hz, 2H); MS (EI) m/z: 453 (M+H⁺).

### Example 458

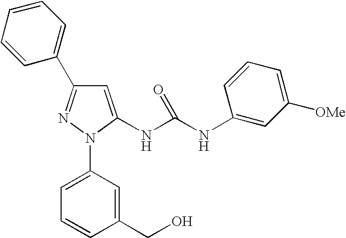

Using the same procedure as for Example 406, Example A35 (120 mg, 0.32 mmol) and 3-methoxyphenyl isocyanate (50 mg, 0.32 mmol) were combined to yield 1-{3-phenyl-1-[3-(hydroxymethyl)phenyl]-1H-pyrazol-5-yl}-3-(3-methoxyphenyl)urea as a white powder (22 mg, 50% yield). ¹H-NMR (DMSO-d₆): δ 9.07 (s, 1H), 9.03 (br s, 1H), 8.52 (s, 1H), 7.85 (m, 2H), 7.56 (s, 1H), 7.1-7.55 (m, 7H), 6.94 (s, 1H), 6.91 (dd, J=1.2, and 8.1 Hz, 1H), 6.56 (dd, J=1.8, and 7.5 Hz, 1H), 5.31 (br s, 1H), 4.61 (br s, 2H), 3.72 (s, 3H); MS (EI) m/z: 415 (M+H⁺).

### Example 459

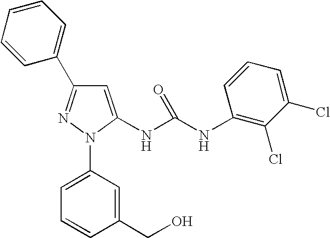

Using the same procedure as for Example 406, Example A35 (120 mg, 0.32 mmol) and 2,3-dichlorophenyl isocyanate (59 mg, 0.32 mmol) were combined to yield 1-{3-phenyl-1-[3-(hydroxymethyl)phenyl]-1H-pyrazol-5-yl}-3-(2,3-dichlorophenyl)urea as a white powder (29 mg, 71% yield). ¹H-NMR (DMSO-d₆): δ 9.37 (s, 1H), 8.85 (s, 1H), 8.08 (m, 1H), 7.85 (m, 2H), 7.58 (s, 1H), 7.3-7.55 (m, 8H), 6.95 (s, 1H), 5.38 (t, J=5.7 Hz, 1H), 4.61 (d, J=5.7 Hz, 2H); MS (EI) m/z: 453 (M+H⁺).

### Example 460

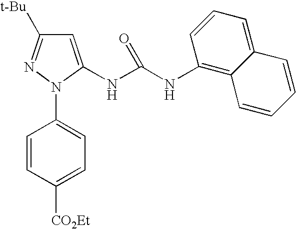

Using the same procedure as for Example 304, Example A22 (4.86 g, 15 mmol) and 1-isocyanato-naphthalene (3.38 g, 20 mmol) were combined to afford ethyl 4-{3-t-butyl-5-[3-(naphthalen-1-yl)ureido]-1H-pyrazol-1-yl}benzoate (1.45 g, 22% yield), which was used without further purification.

### Example 461

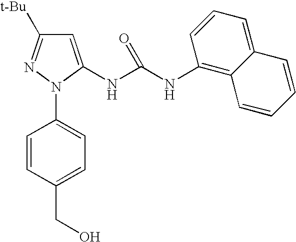

Using the same procedure as for Example 303, Example 460 (1.8 g, 3.21 mmol) was reduced to afford 1-{3-t-butyl-1-[4-(hydroxymethyl)phenyl]-1H-pyrazol-5-yl}-3-(naphthalen-1-yl)urea (1.2 g, 90% yield), which was used without further purification.

### Example 462

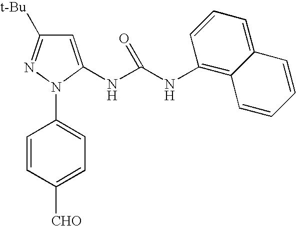

To a solution of Example 461 (200 mg, 0.48 mmol) in fresh CH₂Cl₂ was added powder activated MnO₂ (1.0 g, 12 mmol) and the resulting mixture was stirred at RT overnight. After filteration through, the filtrate was concentrated to afford 1-[3-t-butyl-1-(4-formylphenyl)-1H-pyrazol-5-yl]-3-(naphthalen-1-yl)urea (180 mg, 91% yield), which was used for the next step without further purification.

### Example 463

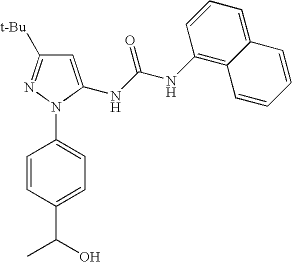

To a solution of Example 462 (100 mg, 0.24 mmol) in fresh THF (40 mL) was added dropwise a solution of methylmagnesium bromide (0.86 mL, 1.4 mol/L in toluene/THF) at 0° C. under N₂. After stirring for 1 h, the resulting mixture was allowed to rise to RT and stirred for 1 h. The reaction mixture was quenched by addition of aqueous solution of HCl (1 mol/L, 50 mL) and extracted with EtOAc (3×50 mL). The combined organic layers were washed with brine, dried (Na₂SO₄), filter, concentrated and purified via column chromatography to afford 1-{3-t-butyl-1-[4-(1-hydroxyethyl)phenyl]-1H-pyrazol-5-yl}-3-(naphthalen-1-yl)urea (55 mg, 54% yield). ¹H-NMR (300 MHz, DMSO-d₆): δ 9.05 (s, 1H), 8.80 (s, 1H), 7.98-7.42 (m, 1H), 6.39 (s, 1H), 4.79 (q, J=6.6 Hz, 1H), 1.36 (d, J=6.6 Hz, 3H), 1.27 (s, 9H).

### Example 464

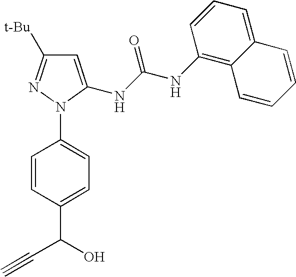

To a solution of Example 462 (100 mg, 0.24 mmol) in fresh THF (40 mL) was added dropwise a solution of ethynylmagnesium bromide (2.42 mL, 0.5 mol/L in toluene/THF) at 0° C. under N₂. After stirred for 1 h, the resulting mixture was allowed to rise to RT and stirred for 1 h. The reaction mixture was quenched by addition of aqueous solution of HCl (1 mol/L, 50 mL) and extracted with EtOAc (3×100 mL). The combined organic layers were washed with brine, dried (Na₂SO₄), filter, concentrated and purified via column chromatography to afford 1-{3-t-butyl-1-[4-(1-hydroxyprop-2-ynyl)phenyl]-1H-pyrazol-5-yl}-3-(naphthalen-1-yl)urea (40 mg, 39% yield). ¹H-NMR (300 MHz, DMSO-d₆): δ 9.05 (br s, 1H), 8.83 (br s, 1H), 8.00 (d, J=7.8 Hz, 1H), 7.90 (d, J=9 Hz, 2H), 7.65-7.42 (m, 8H), 6.40 (s, 1H), 5.43 (d, J=2.1 Hz, 1H), 3.53 (d, J=2.4 Hz, 1H), 1.27 (s, 9H).

### Example 465

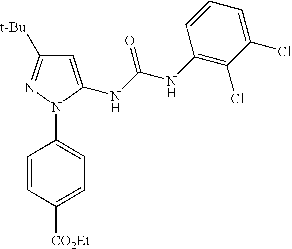

Using the same procedure as for Example 304, Example A22 (1 g, 3.09 mmol) and 1,2-dichloro-3-isocyanato-benzene (0.7 g, 3.71 mmol) were combined to afford ethyl 4-{3-t-butyl-5-[3-(2,3-dichlorophenyl)ureido]-1H-pyrazol-1-yl}benzoate (0.7 g, 48% yield). ¹H-NMR (300 MHz, DMSO-d₆): δ 9.20 (br s, 1H), 8.77 (br s, 1H), 8.04 (m, 1H), 7.44 (br s, 4H), 7.29-7.26 (m, 2H), 6.36 (s, 1H), 4.31 (q, J=7.2 Hz, 2H), 1.27 (s, 9H), 1.26 (t, J=7.2 Hz, 3H).

### Example 466

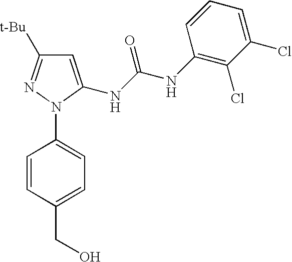

Using the same procedure as for Example 303, Example 465 (80 mg, 0.17 mmol) was reduced to afford 1-{3-t-butyl-1-[4-(hydroxymethyl)phenyl]-1H-pyrazol-5-yl}-3-(2,3-dichloro-phenyl)urea (50 mg, 68% yield). ¹H-NMR (300 MHz, DMSO-d₆): δ 9.20 (br s, 1H), 8.77 (br s, 1H), 8.04 (m, 1H) 7.45 (br s, 4H), 7.30-7.25 (m, 2H), 6.36 (s, 1H), 4.55 (s, 2H), 1.27(s, 9H).

### Example 467

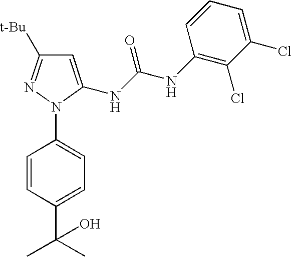

To a solution of Example 465 (100 mg, 0.21 mmol) in fresh THF (10 mL) was added dropwise a solution of methylmagnesium bromide (1.5 mL, 1.4 mol/L in toluene/THF) at 0° C. under N₂. After stirring for 1 h, the resulting mixture was allowed to rise to RT and stirred for 1 h. The reaction mixture was quenched by addition of aqueous solution of HCl (5 mL, 1 M) and the mixture was extracted with EtOAc (3×). The combined organic layers were washed with brine, dried (Na₂SO₄), filter, concentrated and purified via column chromatography to afford 1-{3-t-butyl-1-[4-(2-hydroxypropan-2-yl)phenyl]-1H-pyrazol-5-yl}-3-(2,3-dichlorophenyl)urea (50 mg, 52% yield). ¹H-NMR (300 MHz, DMSO-d₆): δ 9.25 (br s, 1H), 8.79 (br s, 1H), 8.03 (m, 1H), 7.60 (d, J=8.4 Hz, 2H), 7.42 (d, J=8.4 Hz, 2H), 7.30-7.28 (m, 2H), 6.36 (s, 1H), 1.45 (s, 6H), 1.25 (s, 9H)

### Example 468

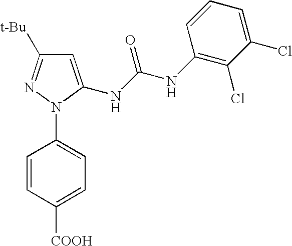

Using the same procedure as for Example 306, Example 465 (80 mg, 0.17 mmol) was saponified to afford 4-{3-t-butyl-5-[3-(2,3-dichlorophenyl)ureido]-1H-pyrazol-1-yl}benzoic acid (60 mg, 79% yield). ¹H-NMR (300 MHz, DMSO-d₆): δ 9.39 (br s, 1H), 8.78 (br s, 1H), 8.07-8.02 (m, 3H), 7.68 (d, J=8.4 Hz, 2H), 7.29 (d, J=7.8 Hz, 1H), 6.41 (s, 1H), 1.21 (s, 9H)

### Example 469

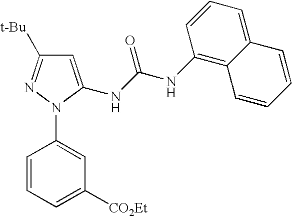

Using the same procedure as for Example 304, Example A2 (4.86 g, 15 mmol) and 1-isocyanato-naphthalene (3.38 g, 20 mmol) were combined to afford ethyl 3-{3-t-butyl-5-[3-(naphthalen-1-yl)ureido]-1H-pyrazol-1-yl}benzoate (1.27 g, 19% yield).

### Example 470

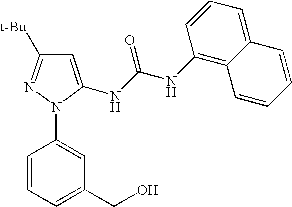

Using the same procedure as for Example 303, Example 469 (1.46 g, 3.21 mmol) was reduced to afford 1-{3-t-butyl-1-[3-(hydroxymethyl)phenyl]-1H-pyrazol-5-yl)-3-(naphthalen-1-yl)-urea (1.1 g, 83% yield), which was used without further purifications.

### Example 471

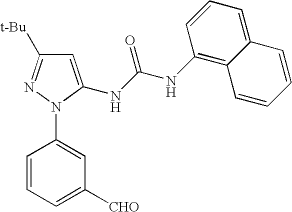

Using the same procedure as for Example 469, Example 470 (200 mg, 0.48 mmol) was oxidized to afford 1-[3-t-butyl-1-(3-formylphenyl)-1H-pyrazol-5-yl]-3-(naphthalen-1-yl)urea (180 mg, 91% yield), which was used without further purification.

### Example 472

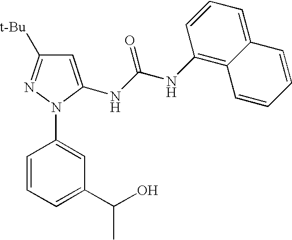

Using the same procedure as for Example 463, Example 471 (100 mg, 0.24 mmol) was oxidized to afford 1-{3-t-butyl-1-[3-(1-hydroxyethyl)phenyl]-1H-pyrazol-5-yl}-3-(naphthalen-1yl)urea (35 mg, 34% yield). ¹H-NMR (300 MHz, DMSO-d₆): δ 9.03 (br s, 1H), 8.89 (br s, 1H), 8.11-7.40 (m, 1H), 6.39 (s, 1H), 3.31 (br s, 1H), 2.51 (d, J=4.8 Hz, 3H), 1.28 (s, 9H).

### Example 473

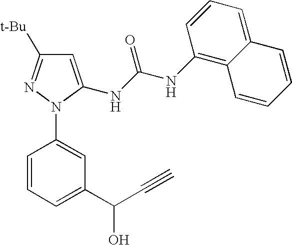

Using the same procedure as for Example 464, Example 471 (100 mg, 0.24 mmol) was reduced to afford 1-{3-t-butyl-1-[3-(1-hydroxyprop-2-ynyl)phenyl]-1H-pyrazol-5-yl}-3-(naphthalen-1-yl)urea (10 mg, 9.5% yield). ¹H-NMR (300 MHz, CDCl₃): δ 7.84 (d, J=8.1 Hz, 2H), 7.71 (d, J=7.8 Hz, 2H), 7.60-7.37 (m, 5H), 7.19 (m, 1H), 6.64 (s, 1H), 5.38 (br s, 1H), 2.65 (s, 1H), 2.60 (d, J=2.1 Hz, 1H), 1.36 (s, 9H).

### Example 474

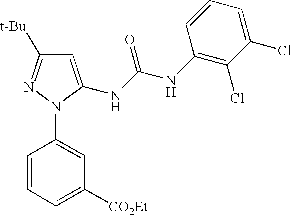

Using the same procedure as for Example 304, Example A2 (1 g, 3.09 mmol) and 1,2-dichloro-3-isocyanato-benzene (0.7 g, 3.71 mmol) were combined to afford ethyl 3-{3-t-butyl-5-[3-(2,3-dichlorophenyl)ureido]-1H-pyrazol-1-yl}benzoate (0.6 g, 41% yield). ¹H-NMR (300 MHz, DMSO-d₆): δ 9.24 (br s, 1H), 8.70 (br s, 1H), 8.05 (t, J=1.8 Hz, 1H), 8.00 (t, J=5.1 Hz, 1H), 7.97-7.93 (m, 1H), 7.84-7.80 (m, 1H), 7.67 (t, J=8.1 Hz, 1H), 7.39 (dd, J=4.8 Hz, 2H), 6.39 (s, 1H), 4.31 (q, J=7.2 Hz, 2H), 1.27 (s, 9H), 1.26 (t, J=7.2 Hz, 3H).

### Example 475

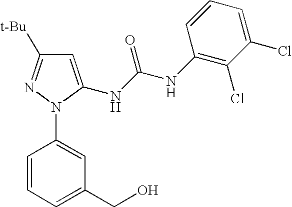

Using the same procedure as for Example 303, Example 474 (80 mg, 0.17 mmol) was reduced to afford 1-[3-t-butyl-1-(3-hydroxymethyl-phenyl)-1H-pyrazol-5-yl]-3-(2,3-dichlorophenyl)-urea (50 mg, 68% yield). ¹H-NMR (300 MHz, DMSO-d₆): δ 9.20 (br s, 1H), 8.75 (br s, 1H), 8.04 (dd, J=3.6 and 6 Hz 1H) 7.49-7.44 (m 2H), 7.37-7.32 (m, 2H), 7.30-7.28 (m, 2H), 6.37 (s, 1H), 4.56 (s, 2H), 1.24 (s, 9H).

### Example 476

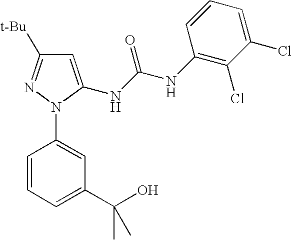

Using the same procedure as for Example 467, Example 474 (100 mg, 0.21 mmol) was reduced to afford 1-{3-t-butyl-1-[3-(2-hydroxypropan-2-yl)phenyl]-1H-pyrazol-5-yl}-3-(2,3-dichlorophenyl)urea (50 mg, 52% yield). ¹H-NMR (300 MHz, DMSO-d₆): δ 9.19 (br s, 1H), 8.72 (br s, 1H), 8.06 (dd, J=36.6 Hz, 1H), 7.58 (m, 1H), 7.46-7.43 (m, 2H), 7.32-7.27 (m, 3H), 6.36 (s,1H), 1.42 (s, 6H), 1.26 (s, 9H).

### Example 477

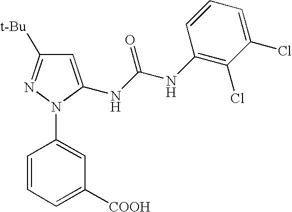

Using the same procedure as for Example 306, Example 474 (80 mg, 0.17 mmol) was saponified to afford 3-{3-t-butyl-5-[3-(2,3-dichlorophenyl)ureido]-1H-pyrazol-1-yl}benzoic acid (60 mg, 79% yield). ¹H-NMR (300 MHz, DMSO-d₆): δ 9.46 (br s, 1H), 8.82 (br s, 1H), 8.05 (br s, 1H), 7.98 (t, J=4.8 Hz, 1H), 7.92 (d, J=7.8 Hz, 1H), 7.80 (d, J=8.7 Hz, I H), 7.63 (t, J=7.8 Hz, 1H), 7.27 (d, J=4.5 Hz, 2H), 6.37 (s, 1H), 1.26 (s, 9H)

### Example A35

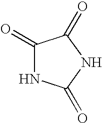

Dry urea (3.0 g) was added to a solution of NaOMe (0.1 mol, in 50 mL of methanol) at RT, stirred for 30 min, after which diethyl oxalate (7.0 g) was slowly added. The mixture was stirred for 1 h, conc. HCl (10 mL) was added and the solution stirred for 10 min. After filtration, the residue was washed twice with a small quantity of methanol, and the combined filtrates were concentrated to yield a white solid imidazolidine-2,4,5-trione which was used without further purification. ¹H NMR (300 MHz, DMSO-d₆): δ 11.8 (s, 2H).

### Example 478

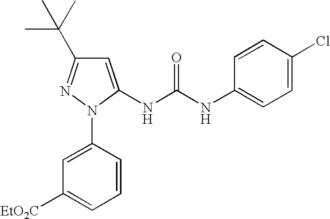

Using the same procedure as for Example 304, Example A2 (10.7 g, 70.0 mmol) and 4-nitrophenyl 4-chlorophenylcarbamate (10 g, 34.8 mmol) were combined to yield ethyl 3-{3-t-butyl-5-[3-(4-chlorophenyl)ureido]-1H-pyrazol-1-yl}benzoate (8.0 g, 52% yield). ¹H NMR (DMSO-d₆): δ 9.11 (s, 1H), 8.47 (s, 1H), 8.06 (m, 1H), 7.93 (d, J=7.6 Hz, 1H), 7.81 (d, J=8.0 Hz, 1H), 7.65 (dd, J=8.0, 7.6 Hz, 1H), 7.43 (d, J=8.8 Hz, 2H), 7.30 (d, J=8.8 Hz, 2H), 6.34 (s, 1H), 4.30 (q, J=6.8 Hz, 2H), 1.27 (s, 9H), 1.25 (t, J=6.8 Hz, 3H); MS (ESI) m/z: 441 (M⁺+H).

### Example A36

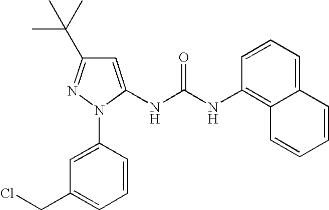

A solution of Example 470 (1.66 g, 4.0 mmol) and SOCl₂ (0.60 mL, 8.0 mmol) in CH₃Cl (100 mL) was refluxed for 3 h and concentrated in vacuo to yield 1-{3-t-butyl-1-[3-chloromethyl)phenyl]-1H-pyrazol-5-yl}-3-(naphthalen-1-yl)urea was obtained as white powder (1.68 g, 97% yield). ¹H NMR (DMSO-d₆): δ 9.26 (s, 1H), 9.15 (s, 1H), 8.42-7.41 (m, 1H), 6.40 (s, 1H), 4.85 (s, 2H), 1.28 (s, 9H). MS (ESI) m/z: 433 (M+H⁺).

### Example 479

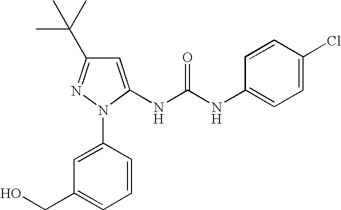

To a stirred solution of Example 478 (1.60 g, 3.63 mmol) in THF (200 mL) was added LiAlH₄ powder (413 mg, 10.9 mmol) at −10° C. under N₂. The mixture was stirred for 2 h and excess LiAlH₄ was quenched by adding ice. The solution was acidified to pH=7 with dilute HCl. Solvents were slowly removed and the solid was filtered and washed with EtOAc (200+100 mL). The filtrate was concentrated to yield 1-{3-t-butyl-1-[3-hydroxymethyl)phenyl]-1H-pyrazol-5-yl}-3-(4-chlorophenyl)urea (1.40 g, 97% yield). ¹H NMR (DMSO-d₆): δ 9.11 (s, 1H), 8.47 (s, 1H), 7.47-7.27 (m, 8H), 6.35 (s, 1H), 5.30 (t, J=5.6 Hz, 1H), 4.55 (d, J=5.6 Hz, 2H), 1.26 (s, 9H); MS (ESI) m/z: 399 (M+H⁺).

### Example A37

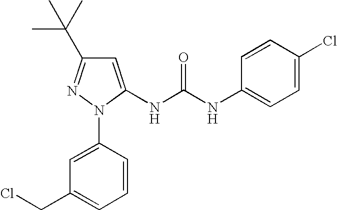

A solution of Example 478 (800 mg, 2.0 mmol) and SOCl₂ (0.30 mL, 4 mmol) in CHCl₃ (30 mL) was refluxed gently for 3 h. The solvent was evaporated in vacuo and the residue was taken up to in CH₂Cl₂ (2×20 mL). After removal of the solvent, 1-{3-t-butyl-1-[3-(chloromethyl)phenyl]-1H-pyrazol-5-yl}-3-(4-chlorophenyl)urea (812 mg, 97% yield) was obtained as white powder. ¹H NMR (DMSO-d₆): δ 9.57 (s, 1H), 8.75 (s, 1H), 7.63 (s, 1H), 7.50-7.26 (m, 7H), 6.35 (s, 1H), 4.83 (s, 2H), 1.27 (s, 9H); MS (ESI) m/z: 417 (M+H⁺).

### Example 480

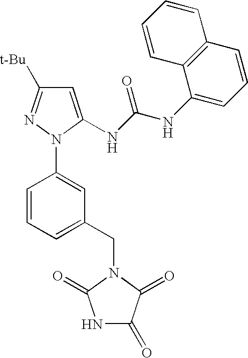

To a mixture of Example A36 (100 mg, 0.23 mmol), K₂CO₃ (64 mg, 0.46 mmol) and KI (10 mg) in DMF (2 mL) was added Example A35 (27.0 mg, 0.23 mmol) at RT. The resulting mixture was stirred at RT overnight. The reaction solution was concentrated in vacuo, and the residue purified by column chromatography to yield 1-{3-t-butyl-1-{3-[(2,4,5-trioxoimidazolidin-1-yl)methyl]phenyl}-1H-pyrazol-5-yl)-3-(naphthalen-1-yl)urea (50 mg, 43% yield). ¹H-NMR (300 MHz, DMSO-d₆): δ 12.10 (s, 1H), 9.06 (s, 1H), 8.93 (s, 1H), 8.03 (d, J=6.0 Hz, 1H), 7.89 (d, J=6.0 Hz, 1H), 7.62-7.41 (m, 8H), 6.41 (s, 1H), 4.73 (s, 2H), 1.27 (s, 9H).

### Example 481

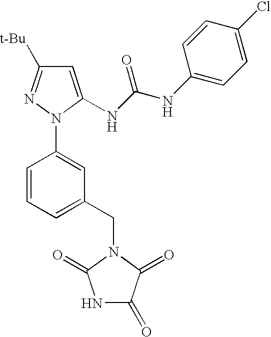

Using the same procedure as for Example 480, Example A37 (100 mg, 0.24 mmol), and Example A35 (29.0 mg, 0.24 mmol) were combined to affored 1-{3-t-butyl-2-{3-[(2,4,5-trioxoimidazolidin-1-yl)methyl]phenyl}-1H-pyrazol-3-yl}-3-(4-chlorophenyl)urea (55 mg, 46% yield). ¹H-NMR (300 MHz, DMSO-d₆): δ 12.10 (s, 1H), 9.00 (s, 1H), 8.45 (s, 1H), 7.50-7.35 (m, 6H), 7.28 (d, J=8.7 Hz, 2H), 6.37 (s, 1H), 4.70 (s, 2H), 1.27 (s, 9H).

### Example A38

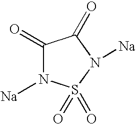

To a solution of NaOMe (0.15 mol, in 60 mL of methanol) was added 7.2 g of sulfamide at RT. The resulting mixture was stirred for 30 min, after which dimethyl oxalate (11.0 g) was added. The suspension mixture was heated to reflux for 16 h, cooled filtered, the precipitate washed with MeOH, and dried under vacuum to yield 1,2,5-thiadiazolidine-3,4-dione 1,1-dioxide as a disodium salt (12.2 g). ¹³C-NMR (300 MHz, D₂O): δ 173 (s, 2 C).

### Example 482

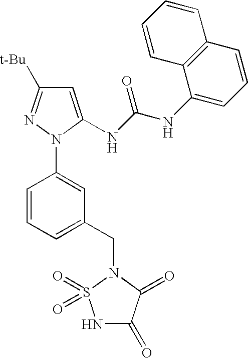

To a mixture of Example A36 (100 mg, 0.23 mmol) in DMF (2 mL) was added Example A38 (89.0 mg, 0.46 mmol) at RT, which was stirred overnight at RT. The reaction solution was concentrated and the residue purified via column chromatography to yield 1-{5-t-butyl-2-[3-(1,1,3,4-tetraoxo-1λ⁶-[1,2,5]thiadiazolidin-2-ylmethyl)phenyl]-2H-pyrazol-3-yl}-3-(naphthalen-1-yl)urea (35 mg, 28% yield). ¹H-NMR (300 MHz, CD₃OD): 7.83-7.92 (m, 2H), 7.64-7.69 (m, 3H), 7.40-7.57 (m, 6H), 6.47 (s, 1H), 4.90 (s, 2H), 1.28 (s, 9H).

### Example 483

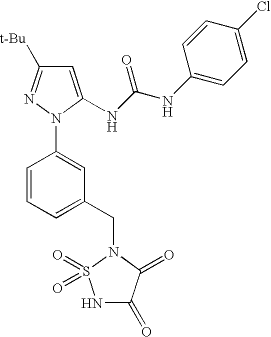

Using the same procedure as for Example 482, Example A37 (100 mg, 0.24 mmol) and Example A38 (91.0 mg, 0.48 mmol) were combined to yield 1-{5-t-butyl-2-[3-(1,1,3,4-tetraoxo-1λ⁶-[1,2,5]thiadiazolidin-2-ylmethyl)phenyl]-2H-pyrazol-3-yl}-3-(4-chlorophenyl)urea (40 mg, 31% yield). ¹H-NMR (300 MHz, DMSO-d₆): δ 8.96 (s, 1H), 8.45 (s, 1H), 7.53 (s, 1H), 7.25-7.46 (m, 7H), 6.35 (s, 1H), 4.69 (s, 2H), 1.25 (s, 9H).

### Example 484

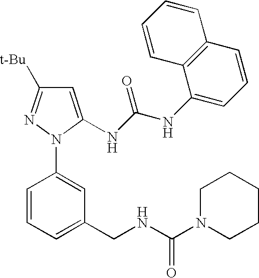

A mixture of Example 410 (100.0 mg, 0.28 mmol) and CDI (48.0 mg, 0.30 mmol) in DMF (2 mL) was stirred at RT for 2 h, and was followed by the addition piperidine (0.05 mL). The resulting mixture was stirred overnight, concentrated in vacuo and the residue purified by preparative HPLC to yield 1-(3-t-butyl-1-{3-[(piperidine-1-carboxamido)methyl]-phenyl}-1H-pyrazol-5-yl)-3-(naphthalen-1-yl)urea (40 mg, 27% yield). ¹H-NMR (300 MHz, DMSO-d₆): δ 9.14 (s, 1H), 8.95 (s, 1H), 8.05 (d, J=8.1 Hz, 1H), 7.88-7.94 (m, 2H), 7.62 (d, J=6.0 Hz, 2H), 7.42-7.53 (m, 6H), 7.27 (d, J=6.9 Hz, 1H), 7.06 (t, J=6.9 Hz, 1H), 6.39 (s, 1H), 4.30 (d, J=5.4 Hz, 2H), 3.25 (br s, 4H), 1.34 (br s, 4H), 1.27 (s, 9H), 1.19-1.24 (m, 2H).

### Example A39

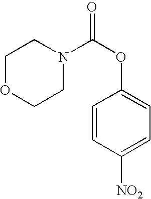

To a solution of 4-nitro phenyl chloroformate (0.243 g, 1.2 mmol) in THF was added morpholine (0.116 mL, 1.2 mmol) at 0° C., and the mixture stirred for 5 h and concentrated to yield 4-nitrophenyl morpholine-4-carboxylate, which was used without further purification. ¹H NMR (300 MHz, DMSO-d₆): δ 8.25 (d, J=9.0 Hz, 2H), 7.43 (d, J=9.0 Hz, 2H), 3.63-3.66 (br s, 4H), 3.59-3.62 (br s, 2H), 3.39-3.45 (br s, 2H).

### Example A40

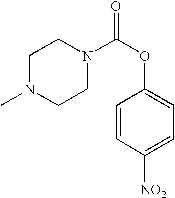

To a solution of 4-nitro phenyl chloroformate (0.243 g, 1.2 mmol) in THF was added 1-methyl-piperazine (0.12 mg, 1.2 mmol) at 0° C., and the mixture was stirred for 5 h and concentrated to yield 4-nitrophenyl 4-methylpiperazine-1-carboxylate, which was used without further purification. ¹H-NMR (300 MHz, DMSO-d₆): δ 8.25 (d, J=9.0 Hz, 2H), 7.42 (d, J=9.0 Hz, 2H), 3.58 (br s, 2H), 3.43 (br s, 2H), 2.47 (br s, 4H), 2.20 (s, 3H).

### Example 485

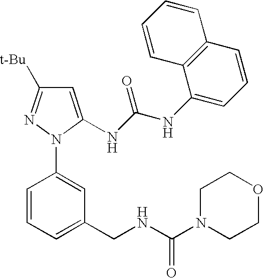

A solution of Example 410 (50 mg, 0.12 mmol) in DMF (1 mL) and Example CCC (30 mg, 0.12 mmol) was heated at 80° C. for overnight and purified via preparative HPLC to yield 30 mg of 1-(3-t-butyl-1-{3-[(morpholine-4-carboxamido)methyl]phenyl}-1H-pyrazol-5-yl)-3-(naphthalen-1-yl)urea (30 mg, 48% yield). ¹H-NMR (300 MHz, DMSO-d₆): δ 9.07 (s, 1H, 8.86 (s, 1H), 8.03 (d, J=8.1 Hz, 1H), 7.88-7.93 (m, 2H), 7.62 (d, J=9.0 Hz, 1H), 7.42-7.53 (m, 6H), 7.29 (d, J=9.0 Hz, 1H), 7.18 (t, J=6.0 Hz, 1H), 6.39 (s, 1H), 4.31 (d, J=5.4 Hz, 2H), 3.47 (t, J=5.1 Hz, 4H), 3.24 (t, J=5.4 Hz, 4H), 1.27 (s, 9H).

### Example 486

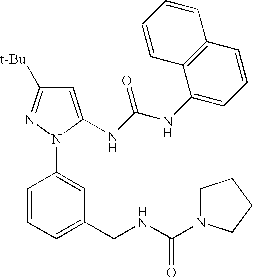

To a solution of pyrrolidine (0.02 mL, 0.24 mmol) in DMF (2 mL) was added NaH (10 mg, 0.24 mmol) at 0° C. The mixture was stirred for 15 min, followed by the addition of Example 410 (100 mg, 0.24 mmol) and CDI (47 mg, 0.28 mmol) in DMF (2 mL). The mixture was stirred overnight, concentrated and purified via preparative HPLC to yield 1-(3-t-butyl-1-{3-[(pyrrolidine-1-carboxamido)methyl]phenyl}-1H-pyrazol-5-yl)-3-(naphthalen-1-yl)urea (35 mg, 29% yield). ¹H-NMR (300 MHz, DMSO-d₆): δ 9.05 (s, 1H), 8.84 (s, 1H), 8.02 (d, J=8.1 Hz, 1H), 7.89-7.94 (m, 2H), 7.62 (d, J=6.9 Hz, 1H), 7.39-7.54 (m, 6H), 7.31 (d, J=7.5 Hz, 1H), 6.70 (s, 1H), 6.40 (s, 1H), 4.29 (d, J=4.8 Hz, 2H), 3.17 (t, J=6.6 Hz, 4H), 1.67 (t, J=6.6 Hz, 4H), 1.27 (s, 9H).

### Example 487

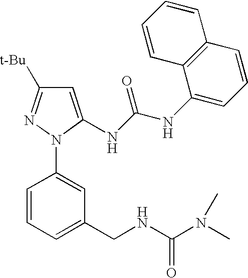

Using the same procedure as for Example 486, Example 410 (100 mg, 0.24 mmol) and dimethylamine (0.02 mg, 0.24 mmol) were combined to yield 35 mg of 1-{3-t-butyl-1-[3-(3,3-dimethylureidomethyl)phenyl]-1H-pyrazol-5-yl}-3-(naphthalen-1-yl)urea (35 mg, 30% yield). N,N-dimethylamino-1-carboxylic acid 3-[3-t-butyl-5-(3-naphthalen-1-yl-ureido)-pyrazol-1-yl]-benzylamide ¹H NMR (300 MHz, DMSO-d₆): δ 9.06 (s, 1H), 8.85 (s, 1H), 8.01 (d, J=9.0 Hz, 1H), 7.87-7.92 (m, 2H), 7.61 (d, J=6.0 Hz, 1H), 7.37-7.54 (m, 6H), 7.28 (d, J=9.0 Hz, 1H), 6.91 (s, 1H), 6.38 (s, 1H), 2.73 (s, 6H), 1.25 (s, 9H).

### Example 488

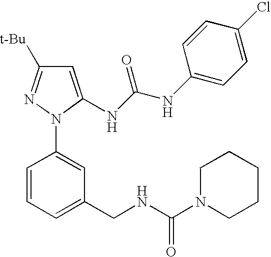

Using the same procedure as for Example 485, Example 390 (100 mg, 0.25 mmol) and piperidine (0.03 mL) were combined to yield 1-(3-t-butyl-1-{3-{(piperidine-1-carboxamido)methyl]phenyl}-1H-pyrazol-5-yl)-3-(4-chlorophenyl)urea (35 mg, 28% yield). ¹H-NMR (300 MHz, DMSO-d₆): δ 9.30 (s, 1H), 8.56 (s, 1H), 7.35-7.55 (m, 8H), 7.15 (t, J=6.0 Hz, 1H), 6.45 (s, 1H), 4.40-4.38 (m, 4H), 1.58-1.60 (m, 2H), 1.46-1.48 (m, 4H), 1.37 (s, 9H).

### Example 489

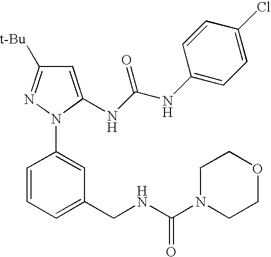

Using the same procedure as for Example 486, Example 390 (100.0 mg, 0.25 mmol) and morpholine (0.028 mL) were combined to yield 1-(3-t-butyl-1-{3-[(morpholine-4-carboxamido)methyl]phenyl}-1H-pyrazol-5-yl)-3-(4-chlorophenyl)urea (25 mg, 20% yield). ¹H-NMR (300 MHz, DMSO-d₆): δ 9.18 (s, 1H), 8.40 (s, 1H), 7.25-7.45 (m, 8H), 7.15 (t, J=6.0 Hz, 1H), 6.35 (s, 1H), 4.29 (d, J=5.4 Hz, 2H), 3.49 (t, J=4.8 Hz, 4H), 3.25 (t, J=4.8 Hz, 4H), 1.25 (s, 9H).

### Example 490

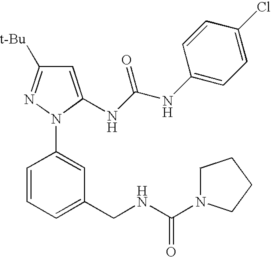

Using the same procedure as for Example 486, Example 390 (100.0 mg, 0.25 mmol) and pyrrolidine (0.025 mL) were combined to yield 1-(3-t-butyl-1-{3-[(pyrrolidine-1-carboxamido)methyl]phenyl}-1H-pyrazol-5-yl)-3-(4-chlorophenyl)urea (30 mg, 24% yield). ¹H-NMR (300 MHz, DMSO-d₆): δ 9.15 (s, 1H), 8.42 (s, 1H), 7.27-7.45 (m, 8H), 6.70 (t, J=6.0 Hz, 1H), 6.35 (s, 1H), 4.27 (d, J=5.4 Hz, 2H), 3.17-3.19 (m, 4H), 1.72-1.74 (m, 4H), 1.25 (s, 9H).

### Example 491

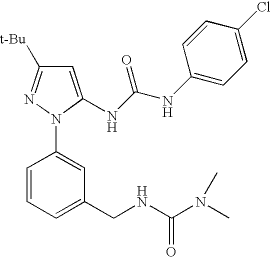

Using the same procedure as for Example 486, Example 390 (100 mg, 0.25 mmol) and dimethylamine (25 mg) were combined to yield 1-{3-t-butyl-1-[3-(3,3-dimethylureidomethyl)phenyl]-1H-pyrazol-5-yl}-3-(4-chlorophenyl)urea (18 mg, 15% yield). ¹H-NMR (300 MHz, DMSO-d₆): δ 9.17 (s, 1H), 8.44 (s, 1H), 7.27-7.43 (m, 8H), 6.80 (t, J=6.0 Hz, 1H), 6.34 (s, 1H), 4.26 (d, J=5.4 Hz, 2H), 2.76 (s, 6H), 1.26 (s, 9H).

### Example 492

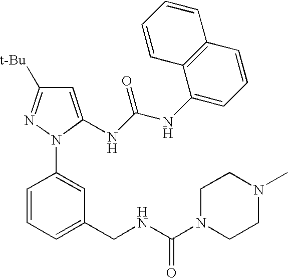

Using the same procedure as for Example 405, Example 410 (50 mg, 0.12 mmol) and Example A40 (32 mg, 0.12 mmol) were combined to yield 1-(3-t-butyl-1-{3-[(1-methylpiperazine-4-carboxamido)methyl]phenyl)-1H-pyrazol-5-yl)-3-(naphthalen-1-yl)urea (35 mg, 54% yield). ¹H-NMR (300 MHz, DMSO-d₆): δ 10.0 (br s, 1H), 9.10 (s, 1H), 8.89 (s, 1H), 8.00-8.02 (d, J=8.0 Hz, 1H), 7.90 (d, J=6.3 Hz, 2H), 7.63 (d, J=9.0 Hz, 1H), 7.44-7.55 (m, 6H), 7.32 (d, J=6.9 Hz, 1H), 6.39 (s, 1H), 4.32 (d, J=5.4 Hz, 2H), 4.05 (br s, 2H), 3.35 (br s, 2H), 2.80-3.10 (m, 4H), 2.74 (s, 3H), 1.27 (s, 9H).

### Example 493

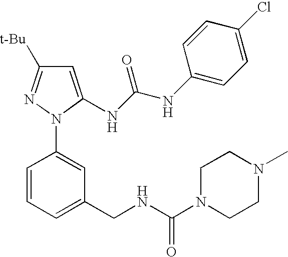

Using the same procedure as for Example 405, Example 390 (100.0 mg, 0.25 mmol) and 1-methyl-piperazine (0.033 mL) were combined to yield 1-(3-t-butyl-1-{3-[(1-methylpiperazine-4-carboxamido)methyl]phenyl}-1H-pyrazol-5-yl)-3-(4-chlorophenyl)urea (40 mg, 31% yield). ¹H-NMR (300 MHz, DMSO-d₆): δ 9.80 (br s, 1H), 9.22 (s, 1H), 8.48 (s, 1H), 7.27-7.43 (m, 8H), 6.34 (s, 1H), 4.30 (d, J=5.4 Hz, 2H), 4.05-4.08 (m, 2H), 3.36-3.38 (m, 2H), 2.81-3.05 (m, 4H), 2.76 (s, 3H), 1.26 (s, 9H).

### Example A41

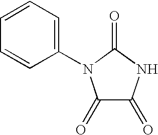

To a solution of aniline (2.51 g, 27 mmol) dissolved in glacial acetic acid (14 mL) and water (28 mL) was slowly added a solution of potassium cyanate (4.4 g, 54 mmol) dissolved in water (35 mL). The mixture stirred for 2 h at RT, filtered, washed with water and dried under reduced pressure to yield phenylurea as a white solid (1.85 g, 50% yield). ¹H NMR (DMSO-d₆): δ 8.47 (s, 1H), 7.38 (dd, J=8.4 Hz, 0.9 Hz, 2H), 7.2 (t, J=7.6 Hz, 2H), 6.88 (t, J=7.6 Hz, 1H), 5.81 (bs, 2H); MS (ESI) m/z: 137 (M+H⁺).

A suspension of Example A15 (0.4 g, 3 mmol) in ether (20 mL) was added oxalylchloride (0.8 g, 6 mmol) and refluxed for 3 h. Solvent was removed under reduced pressure and solid was dried to yield 1-phenylimidazolidine-2,4,5-trione (0.51 g, 89% yield), which was used without purification. ¹H NMR (DMSO-d₆): δ 7.53-7.38 (m, 5H); MS (ESI) m/z: 191 (M+H⁺).

### Example 494

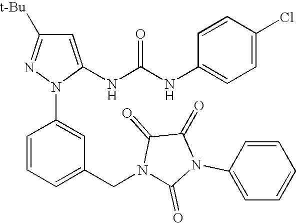

To a solution of triphenyl phosphine (0.23 g, 0.88 mmol) in THF (5 mL) at −20° C. were added di-t-butyl azadicarboxylate (DBAD) (0.2 g, 0.88 mmol), a solution of Example 478 (0.175 g, 0.44 mmol) in THF (5 mL) and Example A41 (0.1 g, 0.53 mmol). The resulting clear yellow solution was heated at 60° C. for 8 h, followed by the further addition of one equivalent of triphenyl phosphine and DBAD and additional heating at 60° C. overnight. One additional equivalent of triphenyl phosphine and DBAD were added and reaction mixture was heated at 60° C. for 3 h. The reaction mixture was concentrated and purified via column chromatography to yield 1-(3-t-butyl-1-(3-[(2,4,5-trioxo-3-phenylimidazolidin-1-yl)methyl]phenyl}-1H-pyrazol-5-yl)-3-(4-chlorophenyl)urea as a white solid (70 mg, 28% yield). ¹H NMR (DMSO-d₆): δ 9.02 (s, 1H), 8.45 (s, 1H), 7.53-7.28 (m, 12H), 6.39 (s, 1H), 4.87 (s, 2H), 1.28 (s, 9H); MS (ESI) m/z: 571 (M+H⁺).

### Example 495

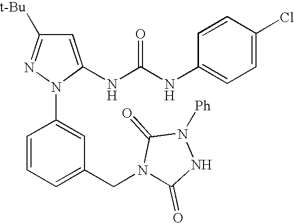

A mixture of 1-phenyl urazole (70 mg, 0.4 mmol), DMF (5 mL) and NaH (5 mg, 0.2 mmol) under Ar at 0° C. was stirred for 30 min. Example A37 (83 mg, 0.2 mmol) was added at 0° C., reaction mixture was warmed to RT, stirred for 8 h, quenched with water (25 mL), and extracted with EtOAc (2×25 mL). The combined organic extracts were washed with water and brine, dried (Na₂SO₄), concentrated under reduced pressure and purified by column chromatography to yield 1-(3-t-butyl-1-{3-[(3,5-dioxo-1-phenyl-1,2,4-triazolidin-4-yl)methyl]phenyl}-1H-pyrazol-5-yl)-3-(4-chlorophenyl)urea as a white solid (85 mg, 77% yield). ¹H NMR (DMSO-d₆): δ 9.06 (s, 1H), 8.49 (s, 1H), 7.48-7.29 (m, 12H), 7.24 (s, 1H), 7.1-7.08 (m, 1H), 6.36 (s, 1H), 4.64 (s, 2H), 1.28 (s, 9H); MS (ESI) m/z: 558 (M+H⁺).

### Example 496

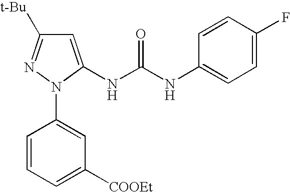

To a solution of Example A2 (0.57 g, 2 mmol) in THF were added pyridine (0.31 g, 4 mmol) 4-fluoro phenyl isocyanate (0.27 g, 2 mmol) and reaction mixture was stirred at RT for 20 h. Then solvent was removed under reduced pressure, and the residue was solidified by stirring with hexane to yield of ethyl 3-{3-t-butyl-5-[3-(4-fluorophenyl)ureido)-1H-pyrazol-1-yl}benzoate as a white solid (0.78 g, 92% yield) ¹H NMR (DMSO-d₆): δ 9.02 (s, 1H), 8.44 (s, 1H), 8.08 (t, J=1.6 Hz, 1H), 7.95 (d, J=8.0 Hz, 1H), 7.83 (dd, J=8 Hz, 1.6 Hz, 1H), 7.67 (t, J=8 Hz, 1H), 7.42-7.39 (m, 2H), 7.09 (t, J=8.8 Hz, 2H), 6.37 (s, 1H), 4.32 (q, J=7.2 Hz, 2H), 1.30-1.28 (m, 12H); MS (ESI) m/z: 425 (M+H⁺).

### Example 497

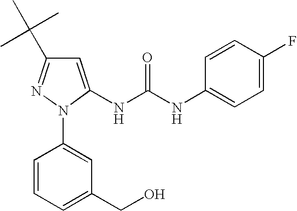

To a solution of Example 496 (0.78 g, 1.8 mmol) in THF (20 mL) was added LAH (5.5 mL of 1M solution in THF) at 0° C. The mixture was warmed to RT, stirred for 1 h, quenched with ice at 0° C. and concentrated under reduced pressure. The residue was acidified with 1M HCl and product was extracted with EtOAc (2×50 mL). The combined organic extracts were washed with brine, dried (Na₂SO₄) and concentrated under reduced pressure to yield 1-{3-t-butyl-1-[3-(hydroxymethyl)phenyl]-1H-pyrazol-5-yl}-3-(4-fluorophenyl)urea as a white solid (0.66 g, 94% yield) ¹H NMR (DMSO-d₆): δ 9.20 (s, 1H), 8.48 (s, 1H), 7.48-7.36 (m, 6H), 7.10 (t, J=8.8 Hz, 2H), 6.37 (s, 1H), 4.58 (s, 2H), 1.28 (s, 9H); MS (ESI) m/z: 383 (M+H⁺).

### Example A42

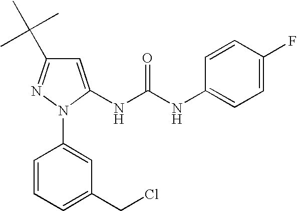

To a solution of Example 496 (0.45 g, 1.2 mmol) in chloroform (20 mL) was added thionyl chloride (0.28 g, 2.4 mmol) and mixture was stirred for 2 h at 65° C. Water was added and organic layer separated. The aqueous layer was extracted with CH₂Cl₂ (1×50 mL) and the combined organic extracts were washed with brine, dried (Na₂SO₄) and concentrated under reduced pressure to yield 1-{3-t-butyl-1-[3-(chloromethyl)phenyl]-1H-pyrazol-5-yl}-3-(4-fluorophenyl)urea as a solid (0.43 g, 96% yield). ¹H NMR (CDCl₃): δ 7.52 (s, 1H), 7.39-7.34 (m, 3H), 7.23-7.19 (m, 2H), 6.97-6.95 (m, 3H), 6.41 (s, 1H), 4.57 (s, ²H), 1.36 (s, 9H); MS (ESI) m/z: 401 (M+H⁺).

### Example 498

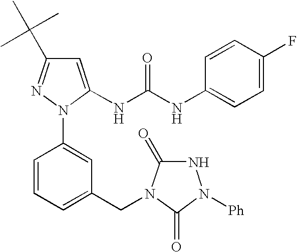

A solution of Example A41 (80 mg, 0.45 mmol), DMF (4 mL) and NaH (5 mg, 0.22 mmol) under Ar at 0° C. was stirred for 30 min. Example A42 (90 mg, 0.22 mmol) was added and the mixture was warmed to RT, stirred for 6 h, quenched with water (20 mL) and extracted with ethyl acetate (2×25 mL). The combined organic extracts were washed with water, brine, dried (Na₂SO₄), concentrated under reduced pressure and purified via column chromatography to yield 1-(3-t-butyl-1-{3-[(3,5-dioxo-1-phenyl-1,2,4-triazolidin-4-yl)methyl]phenyl}-1H-pyrazol-5-yl)-3-(4-fluorophenyl)urea as a white solid (65 mg, 53% yield) ¹H NMR (DMSO-d₆): δ 8.96 (s, 1H), 8.44 (s, 1H), 7.49-7.33 (m, 9H), 7.24 (s, 1H), 7.12-7.08 (m, 3H), 6.35 (s, 1H), 4.64 (s, 2H), 1.28 (s, 9H); MS (ESI) m/z: 542 (M+H⁺).

### Example A43

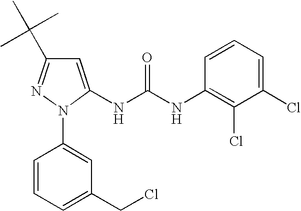

Using the same procedure as for Example A42, Example 474 (0.61 g, 1.4 mmol) was transformed to yield 1-(3-t-butyl-1-(3-(chloromethyl)phenyl)-1H-pyrazol-5-yl)-3-(2,3-dichlorophenyl)urea as a solid (0.6 g, 94% yield). ¹H NMR (CDCl₃): δ 8.12-8.09 (m, 1H), 7.65 (s, 1H), 7.58 (s, 1H), 7.47-7.36 (m, 3H), 7.19-7.17 (m, 2H), 6.95 (br s, 1H), 6.44 (s, 1H), 4.58 (s, 2H), 1.38 (s, 9H); MS (ESI) m/z: 451 (M+H⁺).

### Example 499

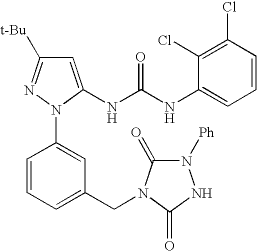

A solution of Example A41 (70 mg, 0.4 mmol), DMF (5 mL) and NaH (5 mg, 0.2 mmol) under Ar at 0° C. was stirred for 30 min, after which Example A43 (90 mg, 0.2 mmol) was added. The mixture was warmed to RT, stirred for 6 h, quench with water (20 mL) and extracted with EtOAc (2×). The combined organic extracts were washed with water, brine, dried (Na₂SO₄), concentrated under reduced pressure and purified via column chromatography to yield 1-(3-t-butyl-1-{3-[(3,5-dioxo-1-phenyl-1,2,4-triazolidin-4-yl)methyl]phenyl}-1H-pyrazol-5-yl)-3-(2,3-dichlorophenyl)urea as a white solid (85 mg, 72% yield). ¹H NMR (DMSO-d₆): δ 9.29 (s, 1H), 8.73 (s, 1H), 8.07 (dd, J=6.4 Hz, 3.2 Hz, 1H), 7.50-7.44 (m,4H), 7.37-7.25 (m, 5H), 7.12-7.10 (m, 1H), 6.38 (s, 1H), 4.64 (s, 2H), 1.28 (m, 9H); MS (ESI) m/z: 592 (M+H⁺).

### Example 500

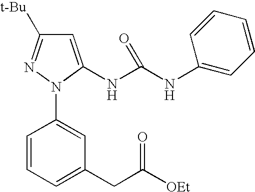

To a solution of Example A9 (2 g, 6.6 mmol) and triethylamine (2.2 g, 20 mmol) in THF (50 mL) was added a solution of benzene isocyanate (890 mg, 7.4 mmol) in THF (5 mL) dropwise at 0° C. under N₂ atmosphere. The mixture was warmed to RT, stirred overnight and then poured into ice aqueous solution of HCl (1 mol/L). The reaction mixture was extracted by CH₂Cl₂ (3×100 mL). The combined organic layers were washed with brine, dried (Na₂SO₄), filtered and concentrated to yield a crude solid which was purified by column chromatography to afford ethyl 2-{3-[3-t-butyl-5-(3-phenylureido)-1H-pyrazol-1-yl]phenyl}acetate (1.5 g, 54% yield). ¹H NMR (300 MHz, DMSO-d₆): δ 8.98 (s, 1H), 8.37 (s, 1H), 7.38-7.35 (m, 5H), 7.25-7.23 (m, 3H), 6.92 (t, J=7.2 Hz, 1H), 6.35 (s, 1H), 4.04 (q, J=7.2 Hz, 2H), 3.72 (s, 2H), 1.24 (s, 9H), 1.15 (t, J=7.2 Hz, 3H); MS (ESI) m/z: 421 (M+H⁺).

### Example 501

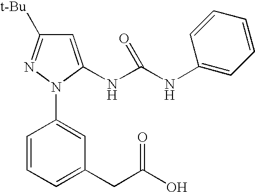

A mixture of compound 500 (1.4 g, 3.3 mmol) in aqueous solution LiOH (2 N, 10 mL) and THF (20 mL) was stirred at RT for 4 h. After removal of the organic solvent, the mixture was extracted with Et₂O. The aqueous solution was acidified with 2 N HCl to pH=4. The precipitate was collected, washed with brine and dried to afford 2-{3-[3-t-butyl-5-(3-phenyl-ureido)-1H-pyrazol-1-yl]phenyl}acetic acid (0.9 g, 70% yield). ¹H NMR (300 MHz, DMSO-d₆): δ 9.07 (s, 1H), 8.40 (s, 1H), 7.39-7.35 (m, 5H), 7.25-7.23 (m, 3H), 6.93 (t, J=7.2 Hz, 1H), 6.35 (s, 1H), 3.62 (s, 2H), 1.24 (s, 9H); MS (ESI) m/z: 392 (M+H⁺).

### Example 502

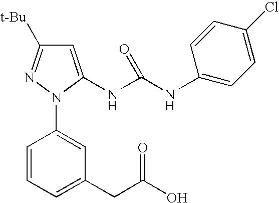

Using the same procedure as for Example 501, Example 423 (2.0 g, 4.4 mmol) was saponified to afford 2-(3-{3-t-butyl-5-[3-(4-chlorophenyl)ureido]-1H-pyrazol-1-yl-}phenyl)acetic acid (1.7 g, 91% yield). ¹H NMR (300 MHz, DMSO-d₆): δ 9.18 (s, 1H), 8.46 (s, 1H), 7.42-7.37 (m, 6H), 7.28-7.25 (m, 3H), 6.33 (s, 1H), 3.64 (s, 2H), 1.24 (s, 9H); MS (ESI) m/z: 427 (M+H⁺).

### Example 503

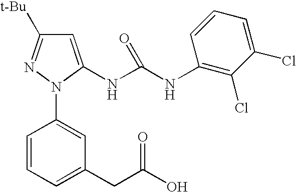

Using the same procedure as Example 501, Example 353 (2.0 g, 4.4 mmol) was saponified i to afford 2-(3-{3-t-butyl-5-[3-(2,3-dichlorophenyl)ureido]-1H-pyrazol-1-yl}phenyl)acetic acid (1.7 g, 84% yield). ¹H NMR (300 MHz, DMSO-d₆): δ 9.26 (s, 1H), 8.76 (s, 1H), 8.03 (m, 1H), 7.48-7.35 (m, 3H), 7.27-7.25 (m, 3H), 6.36 (s, 1H), 3.64 (s, 2H), 1.24 (s, 9H); MS (ESI) m/z: 461 (M+H⁺).

### Example 504

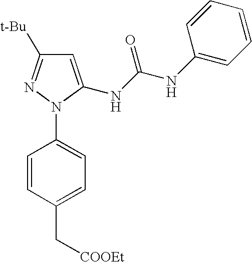

Using the same procedure as for Example 304, Example A1 (2 g, 5.9 mmol) and benzene isocyanate (890 mg, 7.5 mmol) were combined to afford ethyl 2-{4-[3-t-butyl-5-(3-phenylureido)-1H-pyrazol-1-yl]phenyl}acetate (1.8 g, 73% yield). ¹H NMR (400 MHz, DMSO-d₆): δ 8.97 (s, 1H), 8.36 (s, 1H), 7.46-7.36 (m, 6H), 7.23 (t, J=8.1 Hz, 2H), 6.93 (t, J=7.5 Hz, 1H), 6.34 (s, 1H), 4.07 (q, J=7.2 Hz, 2H), 3.71 (s, 2H), 1.24 (s, 9H), 1.17 (t, J=7.2 Hz, 3H); MS (ESI) m/z: 421(M+H⁺).

### Example 505

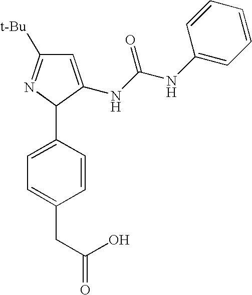

Using the same procedure as for Example 306, Example 505 (1.7 g, 4.0 mmol) was saponified afford 2-{4-[5-t-butyl-3-(3-phenylureido)-2H-pyrrol-2-yl]phenyl)acetic acid (1.1 g, 70% yield). ¹H NMR (300 MHz, DMSO-d₆): δ 9.04 (s, 1H), 8.42 (s, 1H), 7.45-7.36 (m, 6H), 7.23 (t, J=8.1 Hz, 2H), 6.93 (t, J=7.2 Hz, 1H), 6.34 (s, 1H), 3.62 (s, 2H), 1.25 (s, 9H); MS (ESI) m/z: 392 (M+H⁺)

### Example 506

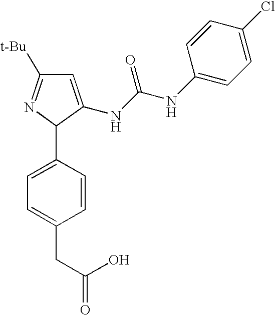

Using the same procedure as for Example 306, Example 420 (1.7 g, 4.0 mmol) was saponified to afford 2-(4-{5-t-butyl-3-[3-(4-chlorophenyl)ureido]-2H-pyrrol-2-yl}-phenyl)acetic acid (1.1 g, 65% yield). ¹H NMR (300 MHz, DMSO-d₆): δ 11.56 (s, 1H), 11.24 (s, 1H), 7.52-7.47 (m, 4H), 7.28 (d, J=8.4 Hz, 2H), 7.19 (d, J=8.4 Hz, 2H), 6.17 (s, 1H), 3.31 (s, 2H), 1.26 (s, 9H); MS (ESI) m/z: 426 (M+H⁺).

### Example 507

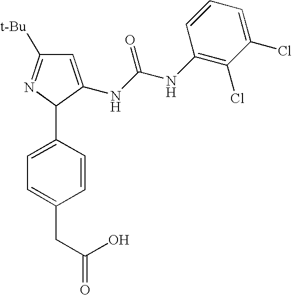

Using the same procedure as for Example 501, Example 424 (2.0 g, 4.1 mmol) was saponified to afford 2-(4-{5-t-butyl-3-[3-(2,3-dichlorophenyl)ureido]-2H-pyrazol-1-yl}phenyl)acetic acid (1.5 g, 80% yield). ¹H NMR (300 MHz, DMSO-d₆): δ 9.70 (s, 1H), 9.00 (s, 1H), 7.98 (m, 1H), 7.46 (d, J=8.4 Hz, 2H), 7.35 (d, J=8.4 Hz, 2H), 7.25-7.24 (m, 2H), 6.30 (s, 1H), 3.61 (s, 2H), 1.23 (s, 9H); MS (ESI) m/z: 461(M+H⁺)

### Example A44

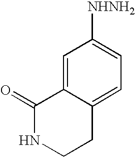

To a solution of phenethylamine (60.5 g, 0.5 mol) and sodium carbonate (63.6 g, 0.6 mol) in ethyl acetate/water (800 mL, 4:1) was added ethyl chloroformate dropwise (65.1 g, 0.6 mol) at 0° C. during a period of 1 h. The mixture was warmed to RT and stirred for an additional 1 h. The organic phase was separated and the aqueous layer was extracted with EtOAc. The combined organic phases were washed with water and brine, dried (Na₂SO₄), filtered and concentrated to a crude solid, which was purified by flash chromatography to afford ethyl phenethyl-carbamate (90.2 g). ¹H NMR (400 MHz, CDCl₃): δ 7.32-7.18 (m, 5H), 4.73 (br s, 1H), 4.14-4.08 (q, J=6.8 Hz, 2H), 3.44-3.43 (m, 2H), 2.83-2.79 (t, J=6.8 Hz, 2H), 1.26-1.21 (t, J=6.8 Hz, 3H).

A suspension of phenethyl-carbamic acid ethyl ester (77.2 g, 40 mmol) in polyphosphoric acid (300 mL) was heated to 140-160° C. and stirred for 2.5 h. The reaction mixture was cooled to RT, carefully poured into ice-water and stirred for 1 h. The aqueous solution was extracted with EtOAc (3×300 mL). The combined organic phases were washed with water, 5% aqueous potassium carbonate and brine, dried (Na₂SO₄), filtered and concentrated to a crude solid, which was purified by flash chromatography to afford 3,4-dihydro-2H-isoquinolin-1-one (24 g). ¹H NMR (400 MHz, DMSO-d₆): δ 7.91 (br s, 1H), 7.83 (d, J=7.5 Hz, 1H), 7.43 (t, J=7.5 Hz, 1H), 7.33-7.25 (m, 2H), 3.37-3.32 (m, 2H), 2.87 (t, J=6.6 Hz, 2H).

To an ice-salt bath cooled mixture of nitric acid and sulfonic acid (200 mL, 1:1) was added, 4-dihydro-2H-isoquinolin-1-one (15 g, 0.102 mol) dropwise over 15 min. After stirring for 2 h, the resulting mixture was poured into ice-water and stirred for 30 min. The precipitate was filtered, washed with water, dried in air to afford 7-nitro-3,4-dihydro-2H-isoquinolin-1-one (13 g). ¹H NMR (300 MHz, DMSO-d₆): δ 8.53 (d, J=2.4 Hz, 1H), 8.31 (d, J=2.4 Hz, 1H), 8.29 (d, J=2.4 Hz, 1H), 7.62 (d, J=8.4 Hz, 1H), 3.44-3.39 (m, 2H), 3.04 (t, J=6.6 Hz, 2H).

A suspension of 7-nitro-3,4-dihydro-2H-isoquinolin-1-one (11.6 g, 60 mmol) and Pd/C (1.2 g, 10%) in methanol was stirred overnight at RT under an H₂ atmosphere (40 psi). The mixture was filtered through celite and washed with methanol. The filtrate was evaporated by vacuum to afford 8.2 g of 7-amino-3,4-dihydro-2H-isoquinolin-1-one, which was used without further purification.

To a suspension of 7-amino-3,4-dihydro-2H-isoquinolin-1-one (8.1 g, 50 mmol) in concentrated HCl (100 mL) was added a solution of sodium nitrite (3.45 g, 50 mmol) in water dropwise in an ice-water bath at such a rate that the reaction mixture never rose above 5° C. After stirring for 30 min, the resulting mixture was added a solution of SnCl₂ (22.5 g, 0.1 mol) in concentrated HCl (150 mL) dropwise at 0° C. in an ice-water bath. The resulting mixture was stirred for another 2 h at 0° C. The precipitate was collected by suction, washed with ether to afford 7-hydrazino-3,4-dihydro-2H-isoquinolin-1-one (8.3 g), which was used for the next reaction without further purification.

### Example A45

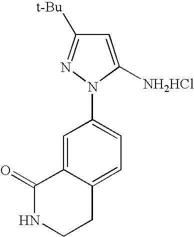

A mixture of Example A44 (8.0 g, 37.6 mmol) and 4,4-dimethyl-3-oxo-pentanenitrile (5.64 g, 45 mmol) in ethanol (100 mL) and concentrated HCl (10 ml) was heated to reflux overnight. After removal of the solvent, the residue was washed with ether to afford 7-(5-Amino-3-t-butyl-pyrazol-1-yl)-3,4-dihydro-2H-isoquinolin-1-one hydrochloride as a yellow solid (11.5 g, 96% yield), which was used without further purification.

### Example 508

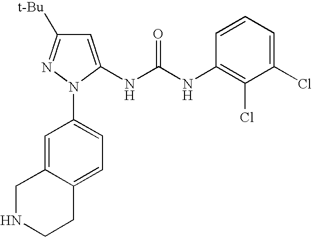

To a suspension of Example A45 (2.0 g, 6.2 mmol) in fresh THF (50 mL) was added a solution of triethylamine (1.7 mL, 12.4 mmol) in THF (5 mL) dropwise at 0° under an N₂ atmosphere. After stirring for 30 min, 1,2-dichloro-3-isocyanato-benzene (1.42 g, 7.5 mmol) in THF (5 mL) was added dropwise via syringe to the mixture. The reaction was warmed to RT and stirred overnight. The reaction was poured onto ice cold aqueous HCl (1.0 N) and extracted with EtOAc (3×100 mL). The combined organic layers were washed with brine, dried (Na₂SO₄), filtered and concentrated to a crude solid, which was purified by flash chromatography to afford 1.2 g 1-[3-t-butyl-1-(1-oxo-1,2,3,4-tetrahydroisoquinolin-7-yl)-1H-pyrazol-5-yl]-3-(2,3-dichlorophenyl)urea (1.2 g, 41% yield). ¹H NMR (300 MHz, CDCl₃): δ 9.08 (br s, 1H), 8.34 (br s, 1H), 8.15 (br s, 1H), 8.02 (m, 1H), 7.60 (br s, 1H), 7.53 (d, J=8.1 Hz, 1H), 7.29 (d, J=8.7 Hz, 1H), 7.15-7.09 (m, 2H), 6.62 (s, 1H), 3.5 (br, 2H), 3.94 (br, 2H), 1.34 (s, 9H).

To a suspension of 1-[3-t-butyl-1-(1-oxo-1,2,3,4-tetrahydroisoquinolin-7-yl)-1H-pyrazol-5-yl]-3-(2,3-dichlorophenyl)urea (120 mg, 0.25 mmol) in fresh THF (50 mL) was added powder LAH (50 mg, 1.27 mmol) by portions in an ice-water bath. The resulting mixture was heated to reflux for 3 h, then cooled in an ice-salt bath and quenched with water and aqueous NaOH. The precipitate was filtered, washed with THF, and the combined filtrates evaporated under reduced pressure to afford 1-[3-t-butyl-(1,2,3,4-tetrahydroisoquinolin-7-yl)-1H-pyrazol-5-yl]-3-(2,3-dichlorophenyl)urea (80 mg, 70% yield). ¹H NMR (300 MHz, CD₃OD): δ 7.98 (t, J=4.8 Hz, 1H), 7.45-7.39 (m, 3H), 7.23 (d, J=5.1 Hz, 2H), 6.41 (s, 1H), 4.41 (s, 2H), 3.52 (t, J=6.3 Hz, 2H), 3.19 (t, J=6.3 Hz, 2H), 1.33 (s, 9H).

### Example 509

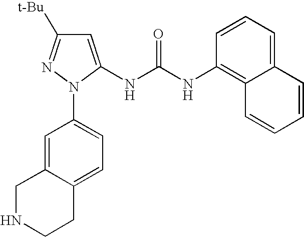

Using the same procedure as for Example 508, Example A45 (2.0 g, 6.2 mmol) and 1-isocyanato-naphthalene (1.27 g, 7.5 mmol) were combined to afford 1-[3-t-butyl-1-(1-oxo-1,2,3,4-tetrahydroisoquinolin-7-yl)-1H-pyrazol-5-yl]-3-(naphthalen-1-yl)urea. ¹H NMR (300 MHz, CDCl₃): δ 8.59 (br s, 1H), 8.32 (br s, 1H), 8.02 (br s,1H), 7.85-7.04 (m, 10H), 6.62 (s, 1H), 3.42 (m, 2H), 2.83 (m, 2H), 1.34 (s, 9H)

Using the same procedure as for Example 405, 1-[3-t-butyl-1-(1-oxo-1,2,3,4-tetrahydroisoquinolin-7-yl)-1H-pyrazol-5-yl]-3-(naphthalen-1-yl)urea. (1.5 g, 3.3 mmol) was reduced to afford 1-[3-t-butyl-1-(1,2,3,4-tetrahydroisoquinolin-7-yl)-1H-pyrazol-5-yl]-3-(naphthalen-1-yl)urea (1.0 g, 69% yield). ¹H NMR (400 MHz, CDCl₃): δ 7.86-6.92 (m, 10H), 6.44 (s, 1H), 3.03 (t, J=6 Hz, 2H), 2.70 (t, J=6 Hz, 2H), 1.33 (s, 9H)

### Example 510

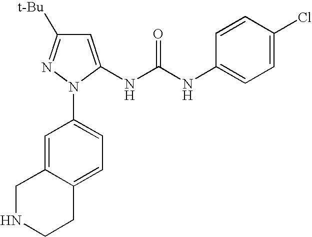

Using the same procedure as for Example 509, Example A45 (2.0 g, 6.2 mmol) and 1-chloro-4-isocyanatobenzene (1.15 g, 7.5 mmol) were combined to afford 1-[3-t-butyl-1-(1-oxo-1,2,3,4-tetrahydroisoquinolin-7-yl)-1H-pyrazol-5-yl]-3-(4-chlorophenyl)urea (1.5 g, 55% yield). ¹H NMR (300 MHz, CDCl₃): δ 9.03 (s, 1H), 8.77 (s, 1H), 7.90 (s, 1H), 7.54 (d, J=7.5 Hz, 1H), 7.30 (d, J=9 Hz, 3H), 7.19 (d, J=9 Hz, 2H), 6.88 (br s, 1H), 6.74 (s, 1H), 3.45 (br s, 2H), 2.88 (t, J=6 Hz, 2H), 1.37 (s, 9H)

Using the same procedure as for Example 405, 1-[3-t-butyl-1-(1-oxo-1,2,3,4-tetrahydroisoquinolin-7-yl)-1H-pyrazol-5-yl]-3-(4-chlorophenyl)urea (1.0 g, 2.3 mmol) was reduced to afford 1-[3-t-butyl-1-(1,2,3,4-tetrahydroisoquinolin-7-yl)-1H-pyrazol-5-yl]-3-(4-chlorophenyl)urea (0.8 g, 82% yield). ¹H NMR (300 MHz, DMSO-d₆): δ 9.13 (br s, 1H), 8.34 (br s, 1H), 7.41-7.12 (m, 7H), 6.31 (s, 1H), 3.88 (s, 2H), 2.95 (t, J=6 Hz, 2H), 2.70 (t, J=6 Hz, 2H), 1.24 (s, 9H).

### Example A46

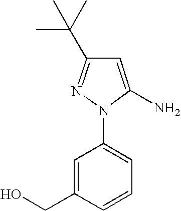

To a stirred solution of Example A2 (19.5 g, 68.0 mmol) in THF (200 mL) was added LiAlH₄ powder (5.30 g, 0.136 mol) at −10° C. under N₂. The mixture was stirred for 2 h at RT and excess LiAlH₄ was destroyed by slow addition of ice. The reaction mixture was acidified to pH=7 with diluted HCl, the solution concentrated under reduced pressure, and the residue was extracted with ethyl acetate. The combined organic extracts were concentrated to yield [3-(5-amino-3-t-butyl-pyrazol-1-yl)-phenyl]-methanol (16.35 g, 98%) as a white powder. ¹H NMR (DMSO-d6): 9.19 (s, 1H), 9.04 (s, 1H), 8.80 (s, 1H), 8.26-7.35 (m, 1H), 6.41 (s, 1H), 4.60 (s, 2H), 1.28 (s, 9H); MS (ESI) m/z: 415 (M+H⁺).

### Example A47

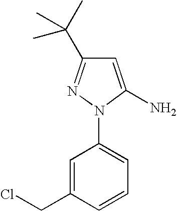

A solution of Example A46 (13.8 g, 56 mmol) and SOCl₂ (8.27 mL, 0.11 mol) in THF (200 mL) was refluxed for 3 h and concentrated under reduced pressure to yield 5-t-butyl-2-(3-chloromethyl-phenyl)-2H-pyrazol-3-ylamine (14.5 g, 98%) as a white powder which was used without further purification. ¹H NMR (DMSO-d6), δ7.62 (s, 1H), 7.53 (d, J=8.0 Hz, 1H), 7.43 (t, J=8.0 Hz, 1H), 7.31 (d, J=7.2 Hz, 1H), 5.38 (s, 1H), 5.23 (br s, 2H), 4.80 (s, 2H), 1.19 (s, 9H). MS (ESI) m/z: 264 (M+H⁺).

### Example A48

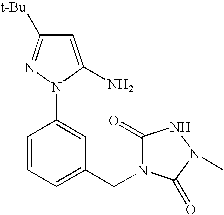

To a suspension of NaH (26 mg, 0.67 mmol) in DMSO (2 mL) was added powder 1-methyl-[1,2,4]triazolidine-3,5-dione (77 mg, 0.67 mmol) at RT under N₂ atmosphere. The resulting mixture was stirred for 30 min and then added to a solution of Example A47 (100 mg, 0.33 mmol) and Et₃N (1 mL) in DMSO (2 mL). After stirring for 3 h, the reaction mixture was quenched with methanol, concentrated and purified by column chromatography to afford 90 mg of 4-[3-(5-amino-3-t-butyl-pyrazol-1-yl)-benzyl]-1-methyl-[1,2,4]triazolidine-3,5-dione.

### Example 511

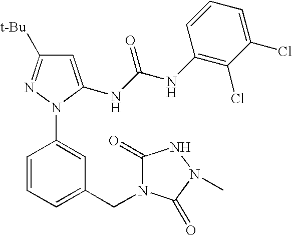

To a suspension of Example A48 (90 mg, 0.26 mmol) and triethylamine (0.5 mL) in fresh THF (10 mL) was added a solution of 1,2-dichloro-3-isocyanato-benzene (95 mg, 0.5 mmol) in THF (2 mL) dropwise through syringe at 0° C. under N₂ atmosphere. The mixture was allowed to rise to RT and stirred overnight. The reaction mixture was quenched with ice-cold aqueous HCl (1 mol/L) and extracted with EtOAc (3×50 mL). The combined organic layers were washed with brine, dried (Na₂SO₄), filtered, concentrated and purified column chromatography to afford 80 mg of 1-{5-t-butyl-2-[3-(1-methyl-3,5-dioxo-[1,2,4]triazolidin-4-ylmethyl)-phenyl]-2H-pyrazol-3-yl}-3-(2,3-dichloro-phenyl)-urea. ¹H-NMR (DMSO-d₆), 511.30 (s, 1H), 9.27 (s, 1H), 8.70 (s, 1H), 8.04 (m, 1H), 7.50-7.46 (m, 3H), 7.28-7.26 (m, 3H), 6.37 (s, 1H), 4.74 (s, 2H), 2.96(s, 3H), 1.25 (s, 9H).

### Example 512

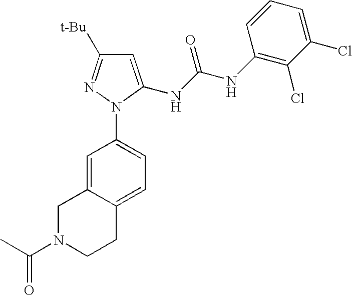

To a solution of Example 508 (100 mg, 0.22 mmol) and triethylamine (60 μL, 0.44 mmol) in CH₂Cl₂ (2 mL) was added acetyl chloride (32 μL, 0.44 mmol) dropwise at 0° C. under N₂. The mixture was warmed to RT and stirred overnight, then poured into ice-cold 1N HCl. The reaction mixture was extracted with CH₂Cl₂ (3×20 mL), and the combined organic extracts were washed with brine, dried (Na₂SO₄), filtered, concentrated and purified via column chromatography to afford 1-[1-(2-acetyl-1,2,3,4-tetrahydroisoquinolin-7-yl)-3-t-butyl-1H-pyrazol-5-yl]-3-(2,3-dichlorophenyl)urea (55 mg, 50% yield). ¹H NMR (300 MHz, DMSO-d₆): 9.16 (m, 1H), 8.74 (s, 1H), 8.00 (s, 1H), 7.20-7.36 (m, 5H), 6.33 (s, 1H), 4.66 (s, 2H), 4.61 (s, 2H), 2.76-2.86 (m, 2H), 2.04 (s, 3H), 1.22 (s, 9H); MS (ESI) m/z: 500 (M+H⁺)

### Example 513

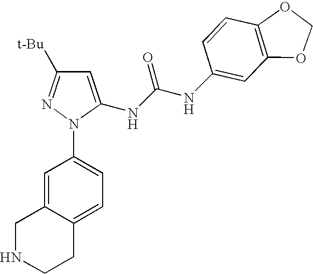

Using the same procedure as for Example 508, Example A45 (285 mg 1.0 mmol) and 5-Isocyanato-benzo[1,3]dioxole (163 mg, 1.0 mmol) were combined to afford 1-benzo[d][1,3]dioxol-5-yl-3-[3-t-butyl-1-(1-oxo-1,2,3,4-tetrahydroisoquinolin-7-yl)-1H-pyrazol-5-yl]urea (200 mg, 45% yield). MS (ESI) m/z: 448 (M+H⁺).

Using the same procedure as for Example 405, 1-benzo[d][1,3]dioxol-5-yl-3-[3-t-butyl-1-(1-oxo-1,2,3,4-tetrahydroisoquinolin-7-yl)-1H-pyrazol-5-yl]urea (120 mg 0.27 mmol) was reduced to afford 1-benzo[d][1,3]dioxol-5-yl-3-[3-t-butyl-1-(1,2,3,4-tetrahydroisoquinolin-7-yl)-1H-pyrazol-5-yl]urea (70 mg, 60% yield). ¹H NMR (300 MHz, DMSO-d₆): δ 9.08 (br s, 2H), 8.99 (s, 1H), 8.43 (s, 1H), 7.40-7.30 (m, 3H), 7.10 (s, 1H), 6.77 (d, J=8.4 Hz, 1H), 6.66 (d, J=8.4 Hz, 1H), 6.28 (s, 1H), 5.91 (s, 2H), 4.30 (br s, 2H), 3.35 (br s, 2H), 2.99 (t, J=6.0 Hz, 2H), 1.25 (s, 9H) MS (ESI) m/z: 434 (M+H⁺)

### Example A49

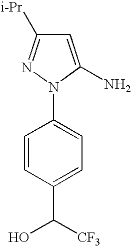

To a solution of 4-nitro-benzaldehyde (15.1 g, 0.1 mol) in THF (100 mL) was added trimethyl-trifluoromethyl-silane (21.3 g, 0.15 mol) and Bu₄NF (500 mg) at 0° C. under N₂. The resulting mixture was stirred at 0° C. for 1 h, then warmed to RT. After stirring at RT for 2 h, the reaction mixture treated with 3.0 N HCl (100 mL), then stirred for 1 h. The reaction was extracted with CH₂Cl₂ with CH₂Cl₂ (3×150 mL). The combined organic extracts were washed with brine, dried (Na₂SO₄), filtered, concentrated and purified via column chromatography to afford 17.2 g of the desired product 2,2,2-trifluoro-1-(4-nitro-phenyl)-ethanol (78%). ¹H NMR (DMSO-d₆): 8.25 (d, J=8.8 Hz, 2H), 7.76 (d, J=8.4 Hz, 2H), 7.15 (d, J=5.6 Hz, 1H), 5.41 (m, 1H).

To a solution of 2,2,2-trifluoro-1-(4-nitro-phenyl)-ethanol (16.0 g, 72 mmol) in methanol (50 mL) was added Pd/C (1.6 g). The mixture was stirred at RT under H₂ at 40 psi for 2 h, then filtered. The filtrate was concentrated to afford 12 g of 1-(4-aminophenyl)-2,2,2-trifluoroethanol (86%), which was used for the next reaction without further purification; MS (ESI) m/z: 192 (M+H⁺)

To a solution of 1-(4-aminophenyl)-2,2,2-trifluoroethanol (12 g, 63 mmol) in conc. HCl (80 mL) was added dropwise an aqueous solution of NaNO₂ (4.3 g, 63 mmol) at 0° C., which was then stirred for 1 h. A solution of SnCl₂ (28.3 g, 0.13 mol) in con.HCl (100 mL) was added dropwise to the mixture at 0° C. The resulting mixture was stirred 0° C. for 2 h, then treated with water and neutralized to pH=8. The reaction mixture was extracted with CH₂Cl₂ (3×150 mL). The combined organic extracts were washed with brine, dried (Na₂SO₄), filtered, concentrated to yield 10 g of 2,2,2-trifluoro-1-(4-hydrazino-phenyl)-ethanol hydrochloride (65%), which was used for the next reaction without further purification; MS (ESI) m/z: 207 (M+H⁺)

A solution of 2,2,2-trifluoro-1-(4-hydrazino-phenyl)-ethanol hydrochloride (1.0 g, 4.1 mmol) and 4-methyl-3-oxo-pentanenitrile (See Example QQ, 620 mg, 5.0 mmol) in ethanol (50 mL) containing conc. HCl (5.0 mL) was heated to reflux for 3 h. After removed the solvent, the residue was purified by column chromatography to afford 1.1 g of 1-(4-(5-amino-3-isopropyl-1H-pyrazol-1-yl)phenyl)-2,2,2-trifluoroethanol (89%); MS (ESI) m/z: 300 (M+H⁺)

### Example 514

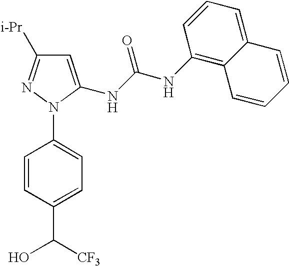

Using the same procedure as for Example 304, Example A49 (150 mg, 0.5 mmol) and 1-isocyanato-naphthalene (85 mg, 0.5 mmol) were combined to afford 50 mg of 1-(1-(4-(2,2,2-trifluoro-1-hydroxyethyl)phenyl)-3-isopropyl-1H-pyrazol-5-yl)-3-(naphthalen-1-yl)urea (21%). ¹H NMR (DMSO-d₆): 9.02 (s, 1H), 8.84 (s, 1H), 7.98 (d, J=7.2 Hz, 1H), 7.90-7.85 (m, 2H), 7.64-7.40 (m, 8H), 6.35 (s, 1H), 5.24 (m, 1H), 2.84 (m, 1H), 1.22 (s, 3H), 1.19 (s, 3H), MS (ESI) m/z: 469 (M+H⁺)

### Example 515

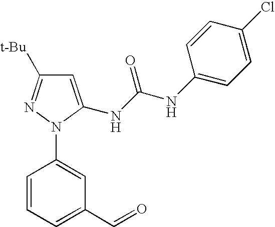

To a solution of Example 479 (500 mg, 1.2 mmol) in CH₂Cl₂ (200 mL) was added MnO₂ (4.3 g, 50 mmol) at RT. The mixture was stirred overnight, then filtered. The filtrate was concentrated to the crude product, which was purified via column chromatography to afford 280 mg of 1-(3-t-butyl-1-(3-formylphenyl)-1H-pyrazol-5-yl)-3-(4-chlorophenyl)urea (56%); MS (ESI) m/z: 397 (M+H⁺).

### Example 516

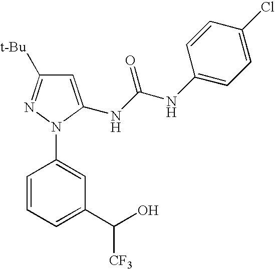

To a solution of Example 515 (200 mg, 0.51 mmol) in THF (20 mL) was added at 0° C. (trifluoromethyl)-trimethylsilane (85 mg, 0.60 mmol) in THF (1 mL) and then TBAF (10 mg) under N₂. The resulting mixture was stirred overnight at RT then treated with HCl (2 N, 1 mL). The reaction mixture was stirred at RT for 30 min, concentrated and the residue dissolved in CH₂Cl₂ (50 mL). The combined organic extracts were washed with with saturated NaHCO₃ and brine, dried (Na₂SO₄), filtered, concentrated and purified via preparative-TLC to afford 30 mg 1-(3-t-butyl-1-(3-(2,2,2-trifluoro-1-hydroxyethyl)phenyl)-1H-pyrazol-5-yl)-3-(4-chlorophenyl)urea (13%). ¹H NMR (400 MHz, DMSO-d₆): 9.49 (s, 1H), 8.73 (s, 1H), 7.66 (s, 1H), 7.52-7.45 (m, 3H), 7.40 (d, J=8.8 Hz, 2H), 7.27 (d, J=8.8 Hz, 2H), 6.99 (s, 1H), 6.32 (s, 1H), 5.24 (m, 1H), 1.26 (s, 9H); MS (ESI) m/z: 467 (M+H⁺).

### Example A50

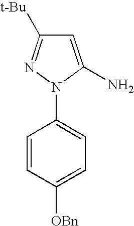

To a mixture of 4-nitro-phenol (10.0 g, 71.9 mmol), K₂CO₃ (19.9 g, 143.9 mmol) and KI (2.6 g, 15.8 mmol) in acetonitrile was added chloromethyl-benzene (10.0 g, 79.1 mmol) at RT. The resultant mixture was heated to reflux for 3 h. After removal of the solvent, the residue was dissolved in EtOAc. The combined organic extracts were washed with brine, dried (Na₂SO₄), filtered and concentrated to afford 14.9 g of 4-benzyloxy-nitrobenzene (90%). ¹H-NMR (400 MHz, CDCl₃): δ 8.20 (d, J=8.0 Hz, 2H), 7.43-7.37 (m, 5H), 7.03 (d, J=8.0 Hz, 2H), 5.17 (s, 2H).

A mixture of 4-benzyloxy-nitrobenzene (13.0 g, 56.5 mmol) and Re—Ni (15.0 g) in EtOH (50 mL) was stirred at RT under 30 psi of H₂. The mixture was stirred at RT overnight, then filtered. The filtrate was concentrated to 10.5 g of 4-benzyloxy-phenylamine (93%) as a brown solid. ¹H-NMR (400 MHz, CDCl₃): δ 7.43 (d, J=7.2 Hz, 2H), 7.38 (t, J=7.2 Hz, 1H), 7.32 (d, J=7.2 Hz, 2H), 6.83 (d, J=8.8 Hz, 2H), 6.65 (d, J=8.8 Hz, 2H), 5.00 (s, 2H), 2.94 (b, 2H); MS(ESI) m/z: 200 (M+H⁺).

To a suspension of 4-benzyloxy-phenylamine (10.0 g, 50.2 mmol) in conc. HCl (50 mL) was added a solution of sodium nitrite (3.46 g, 50.2 mmol) in water in an ice-salt bath. The mixture was stirred at 0° C. for 1 h, after which a solution of SnCl₂ (22.6 g, 100.4 mmol) in conc. HCl was added dropwise at such a rate that the reaction mixture never rose above 5°. The mixture was stirred at RT for 2 h. The precipitate was collected by suction, washed with ethyl ether to afford 9.6 g of (4-benzyloxy-phenyl)-hydrazine hydrochloride (76%). 1H-NMR (DMSO-d₆): δ 10.10 (br s, 3H), 7.43-7.33 (m, 5H), 6.99 (d, J=8.8 Hz, 2H), 6.93 (d, J=8.8 Hz, 2H), 5.03 (s, 2H); MS(ESI) m/z: 215 (M+H⁺).

A solution of (4-benzyloxy-phenyl)-hydrazine hydrochloride (7.50 g, 30 mmol) and 4,4-dimethyl-3-oxo-pentanenitrile (5.0 g, 40 mmol) in alcohol (50 mL) containing conc. HCl (5 mL) was heated to reflux overnight under N₂. After removal of the solvent, the residue was washed with ethyl ether afford 8.2 g of 3-t-butyl-1-(4-(benzyloxy)phenyl)-1H-pyrazol-5-amine (85%). ¹H-NMR (DMSO-d₆): δ 10.20 (br s, 3H), 7.49-7.45 (m, 4H), 7.39 (t, J=7.2 Hz, 1H), 7.34-7.29 (m, 2H), 7.19 (d, J=8.8 Hz, 2H), 5.62 (s, 1H), 5.19 (s, 2H), 1.26 (s, 9H); MS(ESI) m/z: 322 (M+H⁺).

### Example 517

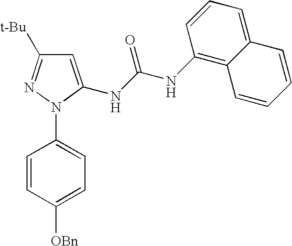

Using the same procedure as for Example 304, Example A50 (650 mg, 2.0 mmol) and 1-isocyanato-naphthalene (338 mg, 2.0 mmol) were combined to afford 470 mg of 1-[2-(4-Benzyloxy-phenyl)-5-t-butyl-2H-pyrazol-3-yl]-3-naphthalen-1-yl-urea (48%). 1H-NMR (DMSO-d6): δ 9.00 (s, 1H), 8.69 (s, 1H), 7.90 (d, J=7.2 Hz, 2H), 7.51-7.37 (m, 12H), 7.16 (d, J=8.8 Hz, 2H), 6.36 (s, 1H), 5.16 (s, 2H), 1.25 (s, 9H); MS (ESI) m/z: 491 (M+H⁺).

### Example 518

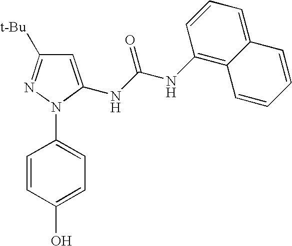

A mixture of Example 517 (300 mg, 0.61 mmol) and Pd/C (60 mg) in methanol (50 mL) was stirred overnight at RT under 50 psi of H₂. After the catalyst was filtered off, the filtrate was concentrated to the crude product, which was purified by column chromatography to afford 200 mg of 1-(3-t-butyl-1-(4-hydroxyphenyl)-1H-pyrazol-5-yl)-3-(naphthalen-1-yl)urea (84%); MS (ESI) m/z: 401 (M+H⁺).

### Example 519

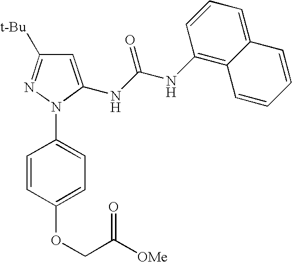

To a mixture of Example 518 (100 mg, 0.25 mmol) and K₂CO₃ (69 mg, 0.50 mmol), KI (50 mg, 0.30 mmol) in acetonitrile (30 mL) was added a solution of chloroacetic acid methyl ester (40 mg, 0.37 mmol) in acetonitrile (2 mL) at RT. The resultant mixture was heated to reflux for 2 h under N₂. After removal of the solvent, the residue was dissolved in CH₂Cl₂ (3×30 mL). The combined organic extracts were washed with brine, dried (Na₂SO₄), filtered, concentrated and purified via preparative HPLC to afford 55 mg of 1-(3-t-butyl-1-(4-(carbomethoxymethyl)oxyphenyl)-1H-pyrazol-5-yl)-3-(naphthalen-1-yl)urea (46%). ¹H-NMR (400 MHz, DMSO-d₆): δ 9.02 (s, 1H), 8.73 (s, 1H), 7.97 (d, J=8.0 Hz, 1H), 7.89 (d, J=8.0 Hz, 2H), 7.62 (d, J=8.0 Hz, 1H), 7.53-7.51 (m, 3H), 7.42 (d, J=8.4 Hz, 2H), 7.08 (d, J=8.8 Hz, 2H), 6.36 (s, 1H), 4.85 (s, 2H), 3.69 (s, 3H), 1.25 (s, 9H); MS (ESI) m/z: 473 (M+H⁺).

### Example 520

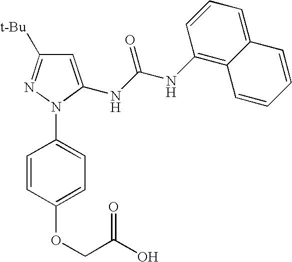

To a solution of Example 519 (20 mg, 0.04 mmol) in THF was added a solution of LiOH (2.0 N, 5 mL) in water at RT. The resultant mixture was stirred at RT for 3 h. After removal of the solvent, the residue was dissolved in DCM. The organic layers were washed with brine dried over Na₂SO₄ and filtered. The filtrate was concentrated to the crude product, which was purified by preparative HPLC to afford 12 mg of 1-(3-t-butyl-1-(4-(carboxy methyl)oxyphenyl)-1H-pyrazol-5-yl)-3-(naphthalen-1-yl)urea (65%). ¹H-NMR (400 MHz, DMSO-d₆): δ 13.04 (br s, 1H), 9.02 (s, 1H), 8.73 (s, 1H), 7.98 (d, J=7.2 Hz, 2H), 7.62 (d, J=8 Hz, 2H), 7.52 (t, J=7.2 Hz, 2H), 7.45-7.43 (m, 3H), 7.06 (d, J=8.8 Hz, 2H), 6.36 (s, 1H), 4.73 (s, 2H), 1.25 (s, 9H); MS (ESI) m/z: 459 (M+H⁺).

### Example 521

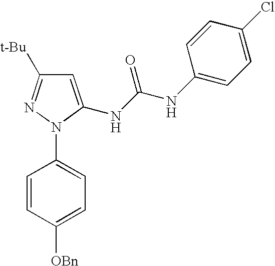

Using the same procedure as for Example 304, Example A50 (650 mg, 2.0 mmol) and 1-chloro-4-isocyanato-benzene (306 mg, 2.0 mmol) were combined to afford 760 mg of 1-(3-t-butyl-1-(4-(benzyloxy)phenyl)-1H-pyrazol-5-yl)-3-(4-chlorophenyl)urea (80%); MS (ESI) m/z: 474 (M+H⁺).

### Example 522

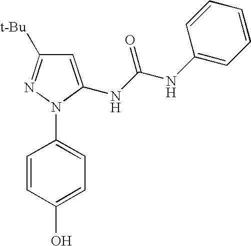

A mixture of Example 521 (500 mg, 1.1 mmol) and Pd/C (100 mg) in methanol (50 mL) was stirred overnight at RT. under 50 psi of H₂. After the catalyst was filtered off, the filtrate was concentrated to the crude product, which was purified by column chromatography to afford 270 mg of 1-(3-t-butyl-1-(4-hydroxyphenyl)-1H-pyrazol-5-yl)-3-phenylurea. ¹H-NMR (300 MHz, DMSO-d₆): δ 9.75 (s, 1H), 8.97 (s, 1H), 8.20 (s, 1H), 7.37 (d, J=7.8 Hz, 2H), 7.24 (d, J=7.8 Hz, 2H), 7.22 (d, J=8.1 Hz, 2H), 6.94 (t, J=7.2 Hz, 1H), 6.87 (d, J=7.8 Hz, 2H), 6.30 (s, 1H), 1.24 (s, 9H); MS (ESI) m/z: 351 (M+H⁺)

### Example 523

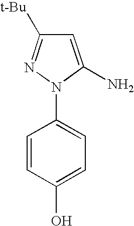

A mixture of Example A50 (650 mg, 2.0 mmol) and Pd/C (130 mg) in methanol (50 mL) was stirred overnight at RT under 50 psi of H₂. After the catalyst was filtered off, the filtrate was concentrated to the crude product, which was purified by column chromatography to afford 380 mg of 4-(3-t-butyl-5-amino-1H-pyrazol-1-yl)phenol (82%); MS (ESI) m/z: 232 (M+H⁺)

### Example 524

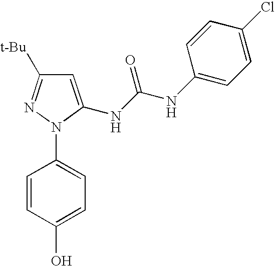

Using the same procedure as for Example 304, Example A50 (350 mg, 1.5 mmol) and 1-chloro-4-isocyanato-benzene (230 mg, 1.5 mmol) were combined to afford 120 mg of 1-(3-t-butyl-1-(4-hydroxyphenyl)-1H-pyrazol-5-yl)-3-(4-chlorophenyl)urea (20%). ¹H-NMR (300 MHz, DMSO-d6): δ 9.82 (br s, 1H), 9.12 (s, 1H), 8.25 (s, 1H), 7.41(d, J=9.0 Hz, 2H), 7.28 (d, J=9.0 Hz, 2H), 7.24 (d, J=8.7 Hz, 2H), 6.86 (d, J=8.7 Hz, 2H), 6.30 (s, 1H), 1.24 (s, 9H); MS (ESI) m/z: 385 (M+H⁺)

### Example 525

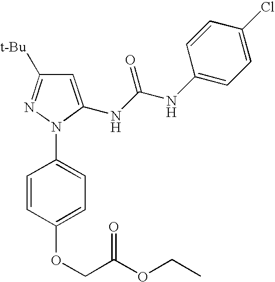

Using the same procedure as for Example 519, Example 524 (120 mg, 0.31 mmol) and chloroacetic acid ethyl ester (76.5 mg, 0.62 mmol) were combined to afford 110 mg of 1-(3-t-butyl-1-(4-(carbomethoxymethyl)oxyphenyl)-1H-pyrazol-5-yl)-3-(4-chlorophenyl-1-yl)urea (75%) as a white solid. ¹H-NMR (300 MHz, DMSO-d₆): δ 9.09 (s, 1H), 8.31 (s, 1H), 7.40 (d, J=5.4 Hz, 2H), 7.34 (d, J=5.4 Hz, 2H), 7.27 (d, J=9.0 Hz, 2H), 7.04 (d, J=9.0 Hz, 2H), 6.30 (s, 1H), 4.81 (s, 2H), 4.16 (q, J=7.2 Hz, 2H), 1.24 (s, 9H), 1.20 (t, J=7.2 Hz, 3H); MS (ESI) m/z: 471 (M+H⁺)

### Example 526

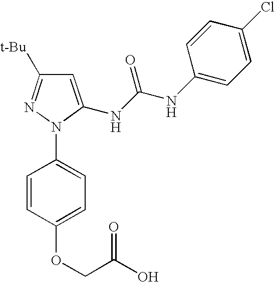

Using the same procedure as for Example 520, Example 525 (60 mg, 0.13 mmol) was saponified to afford 40 mg of 1-(3-t-butyl-1-(4-(carboxymethyl)oxyphenyl)-1H-pyrazol-5-yl)-3-(4-chlorophenyl-1-yl)urea (71%) as a white solid. ¹H-NMR (300 MHz, DMSO-d₆): δ 9.14 (s, 1H), 8.35 (s, 1H), 7.40 (d, J=6.9 Hz, 2H), 7.37 (d, J=6.9 Hz, 2H), 7.27 (d, J=9.0 Hz, 2H), 7.02 (d, J=9.0 Hz, 2H), 6.30 (s, 1H), 4.71 (s, 2H), 1.23 (s, 9H); MS (ESI) m/z: 443 (M+H⁺)

### Example A51

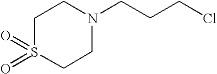

To a mixture of thiomorpholine (500 mg, 3.7 mmol), K₂CO₃ (1.0 g, 7.5 mmol) in acetonitril (50 mL) was added 1-bromo-3-chloro-propane (780 mg, 5.0 mmol) at RT. The mixture was stirred at RT for 3 h. After removal of the solvent, the residue was dissolved in dichloromethane. The organic layer was washed with brine, dried over Na₂SO₄ and filtered. The filtrate was concentrated to the crude product, which was added a solution of HCl MeOH. After removal of the solvent, the residue was washed with Et₂O to afford 510 mg of 4-(3-chloro-propyl)-thiomorpholine-1,1-dioxide (66%). ¹H NMR (400 MHz, D₂O) δ: 3.61 (br s, 4H), 3.31 (br s, 61H), 2.92 (br s, 3H), 2.15 (br s, 2H).

### Example A52

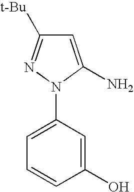

Using the same procedure as for Example A3, m-methoxyphenylhydrazine (40 mmol) and 4,4-dimethyl-3-oxo-pentanenitrile (5.0 g, 40 mmol) were combined to afford 3-(3-t-butyl-5-amino-1H-pyrazol-1-yl)phenol, which was used without further purification.

### Example 527

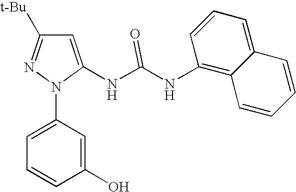

Using the same procedure as for Example 304, Example A52 (2 mmol) and 1-isocyanato-naphthalene (338 mg, 2.0 mmol) were combined to yield 1-(3-t-butyl-1-(3-hydroxyphenyl)-1H-pyrazol-5-yl)-3-(naphthalen-1-yl)urea, which was used without further purification.

### Example 528

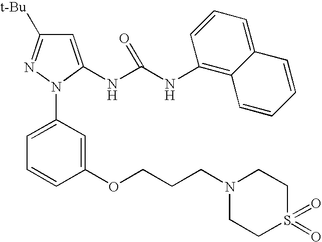

To a solution of Example 527 (100 mg, 0.25 mmol) and K₂CO₃ (68 mg, 0.5 mmol) in acetonitril (10 mL) was added Example A51 (630 mg, 0.30 mmol). The resulting mixture was stirred at 50° C. for 3 h. After removal of the solvent, the residue was dissolved in CH₂Cl₂. The combined organic extracts were washed with brine, dried (Na₂SO₄), filtered, concentrated and purified via preparative HPLC to afford 55 mg of 1-(5-t-butyl-2-{3-[3-(1,1-dioxo-16-thiomorpholin-4-yl)-propoxy]-phenyl}-2H-pyrazol-3-yl)-3-naphthalen-1-yl-urea (38%). ¹H-NMR (400 MHz, CDCl₃) δ: 7.87 (d, J=6.8 Hz, 1H), 7.83 (d, J=7.6 Hz, 1H), 7.74 (d, J=8.4 Hz, 1H), 7.60-7.47 (m, 3H), 7.42 (t, J=7.6 Hz, 1H), 7.11 (m, 1H), 6.95 (s, 2H), 6.79-6.74 (m, 2H), 6.48 (s, 1H), 3.96 (br s, 2H), 3.51 (br s, 4H), 3.01 (br s, 4H), 2.67 (br s, 2H), 1.92 (br s, 2H), 1.35 (s, 9H). MS (ESI) m/z: 576 (M+H⁺).

### Example 529

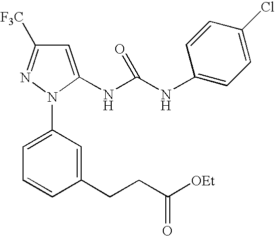

Using the same procedure as for Example 414, 4,4,4-trifluoro-3-oxo-butyronitrile (from Example A6, 1.37 g, 10.0 mmol) was transformed to 4,4,4-trifluoro-3-oxo-butyrimidic acid ethyl ester hydrochloride (1.1 g, 5.0 mmol), which was combined with 1-chloro-4-isocyanato-benzene to afford 970 mg 1-(4-chloro-phenyl)-3-(1-ethoxy-4,4,4-trifluoro-3-oxo-but-1-enyl)-urea (MS (ESI) m/z: 337 (M+H⁺)). This was combined with 3-(3-hydrazino-phenyl)-propionic acid ethyl ester (from Example A14, 500 mg, 2.05 mmol) to yield 650 mg of 3-(3-{5-[3-(4-chloro-phenyl)-ureido]-3-trifluoromethyl-pyrazol-1-yl}-phenyl)-propionic acid ethyl ester. ¹H NMR (400 MHz, DMSO-d₆): 9.19 (s, 1H), 8.70 (s, 1H), 7.52-7.41 (m, 6H), 7.29 (d, J=8.8 Hz, 2H), 6.84 (s, 1H), 4.01 (q, J=7.2 Hz, 2H), 2.92 (t, J=7.6 Hz, 2H), 2.64 (t, J=7.6 Hz, 2H), 1.11 (t, J=7.2 Hz, 3H). MS (ESI) m/z: 481 (M+H⁺).

### Example 530

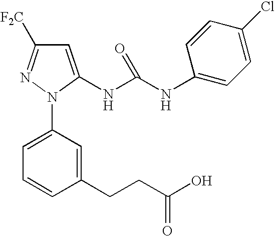

Using the same procedure as for Example 306, Example 529 (150 mg, 0.31 mmol) was saponified to afford 110 mg of 3-(3-(5-(3-(4-chlorophenyl)ureido)-3-(trifluoromethyl)-1H-pyrazol-1-yl)phenyl)propanoic acid. ¹H NMR (400 MHz, DMSO-d₆): 12.15(br s, 1H), 9.36(s, 1H), 8.79(s, 1H), 7.50-7.38 (m, 6H), 7.29 (d, J=8.8 Hz, 2H), 6.84 (s, 1H), 2.90 (t, J=7.2 Hz, 2H), 2.57 (t, J=7.6 Hz, 2H). MS (ESI) m/z: 453 (M+H⁺).

### Example 531

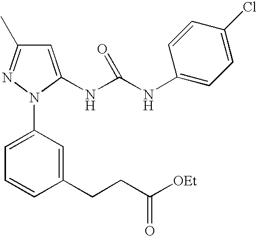

Using the same procedure as for Example 414, 3-oxo-butyronitrile (from Example U, 830 mg, 10.0 mmol) was transformed to 3-oxo-butyrimidic acid ethyl ester hydrochloride (900 mg, 5.4 mmol), which was combined with 1-chloro-4-isocyanato-benzene (1.1 g, 7.2 mmol) to afford 1.3 g of 1-(4-chloro-phenyl)-3-(1-ethoxy-3-oxo-but-1-enyl)-urea (MS (ESI) m/z: 337 (M+H⁺)). This was combined with 3-(3-hydrazino-phenyl)-propionic acid ethyl ester (from Example A14, 500 mg, 2.05 mmol) to yield 750 mg of 3-(3-{5-[3-(4-chloro-phenyl)-ureido]-3-methyl-pyrazol-1-yl}-phenyl)-propionic acid ethyl ester. ¹H NMR (400 MHz, CDCl₃-d₆): 7.39-7.32 (m, 3H), 7.42 (d, J=8.4 Hz, 2H), 7.19 (d, J=8.4 Hz, 2H), 7.13 (d, J=8.0 Hz, 1H), 6.78 (d, J=7.6 Hz, 1H), 6.62 (s, 1H), 6.40 (s, 1H), 4.11 (q, J=7.2 Hz, 2H), 2.86 (t, J=7.6 Hz, 2H), 2.56 (t, J=7.6 Hz, 2H), 1.20 (t, J=7.2 Hz, 3H). MS (ESI) m/z: 427 (M+H⁺).

### Example 532

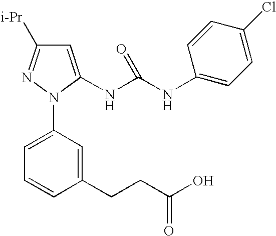

Using the same procedure as for Example 306, Example 416 (200 mg, 0.43 mmol) was saponified to afford 140 mg of 3-(3-(5-(3-(4-chlorophenyl)ureido)-3-isopropyl-1H-pyrazol-1-yl)phenyl)propanoic acid ¹H NMR (400 MHz, CD₄O-d₄): 7.51 (t, J=8.0 Hz, 1H), 7.39-7.35 (m, 5H), 7.24 (d, J=8.8 Hz, 2H), 6.50 (s, 1H), 3.04-2.98 (m, 3H), 2.67 (t, J=7.6 Hz, 2H), 1.31 (d, J=6.8 Hz, 3H). MS (ESI) m/z: 427 (M+H⁺).

All of the reference above identified are incorporated by reference herein. In addition, two simultaneous applications are also incorporated by reference, namely Modulation of Protein Functonalities Ser. No. 10/746,545, filed Dec. 24, 2003 and Anti-Cancer Medicaments, Ser. No. 10/746,607, Filed Dec. 24, 2003.

We claim:

1. A compound having the formula 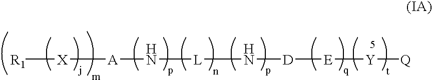
wherein:
R¹ is selected from the group consisting of aryls and heteroaryls;
each X and Y is individually selected from the group consisting of —O—, —S—, —NR₆—, —NR₆SO₂—, —NR₆CO—, alkynyls, alkenyls, alkylenes, —O(CH₂)ₕ—, and —NR₆(CH₂)ₕ—, where each h is individually selected from the group consisting of 1, 2, 3, or 4, and where for each of alkylenes, —O(CH₂)ₕ—, and —NP₆(CH₂)ₕ—, one of the methylene groups present therein may be optionally double-bonded to a side-chain oxo group except that where —O(CH₂)ₕ— the introduction of the side-chain oxo group does not form an ester moiety;
A is selected from the group consisting of aromatic, monocycloheterocyclic, and bicycloheterocyclic rings;
D is phenyl or a five- or six-membered heterocyclic ring selected from the group consisting of pyrazolyl, pyrrolyl, imidazolyl, oxazolyl, thiazolyl, furyl, oxadiazolyl, thiadiazolyl, thienyl, pyridyl, and pyrimidyl;
E is selected from the group consisting of phenyl, pyridinyl, and pyrimidinyl;
L is selected from the group consisting of —C(O)— and —S(O)₂—;
j is 0 or 1;
m is 0 or 1;
n is 0 or 1;
p is 0 or 1;
q is 0 or 1;
t is 0 or 1;
Q is selected from the group consisting of 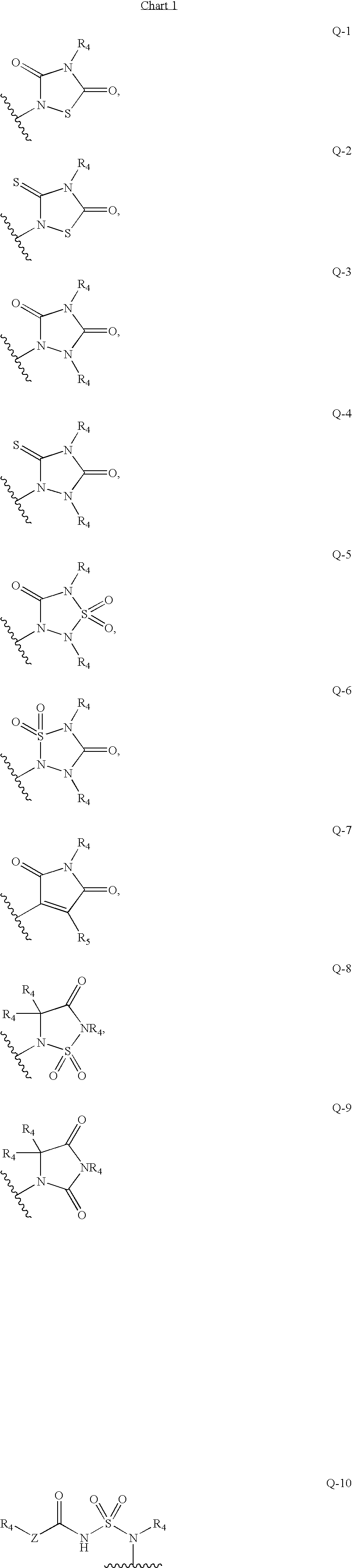 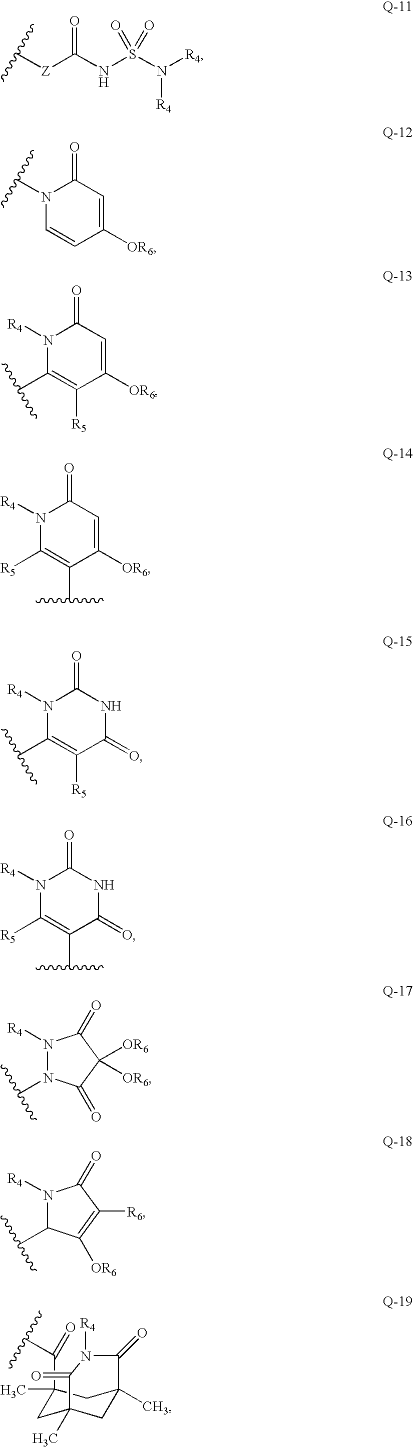 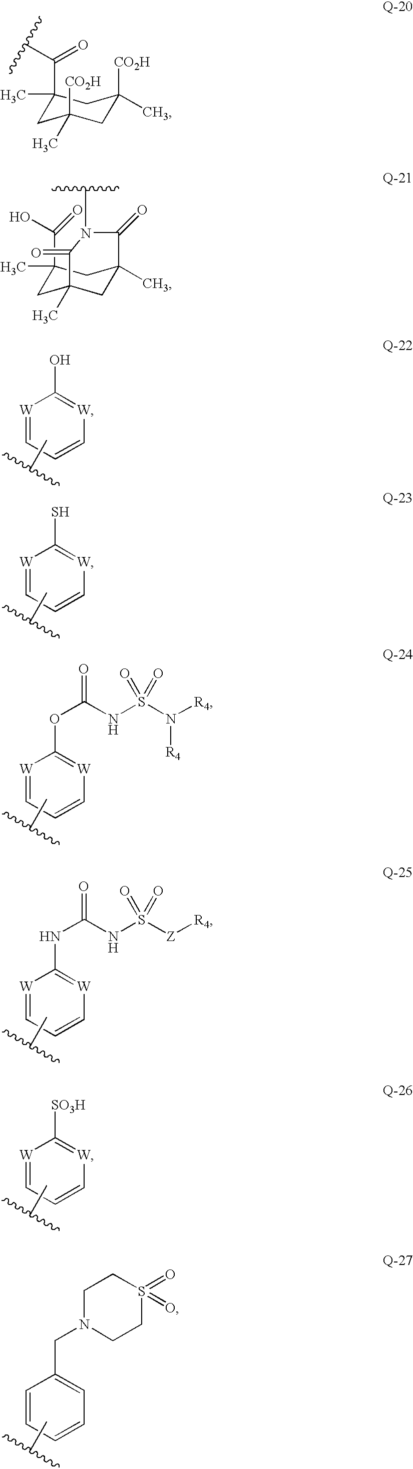 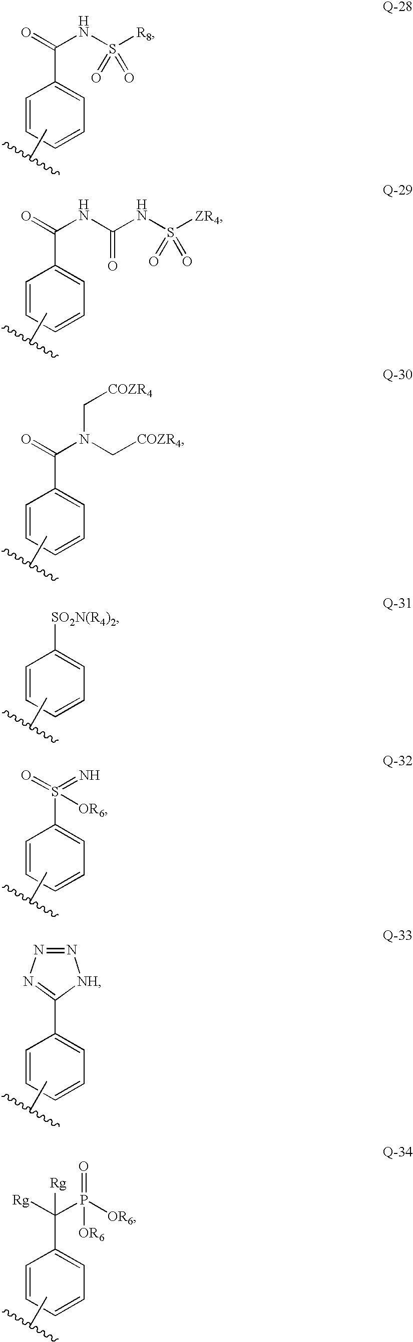 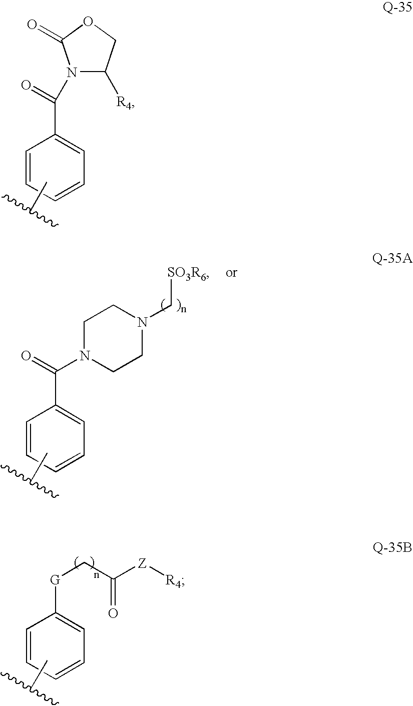
each R₄ group is individually selected from the group consisting of —H, alkyls, aminoalkyls, alkoxyalkyls, aryls, aralkyls, heterocyclyls, and heterocyclylalkyls except when the R₄ substituent places a heteroatom on an alpha-carbon directly attached to a ring nitrogen on Q;
when two R₄ groups are bonded with the same atom, the two R₄ groups optionally form an alicyclic or heterocyclic 4-7 membered ring;
each R₅ is individually selected from the group consisting of —H, alkyls, aryls, heterocyclyls, alkylaminos, arylaminos, cycloalkylaminos, heterocyclylaminos, hydroxys, alkoxys, aryloxys, alkylthios, arylthios, cyanos, halogens, perfluoroalkyls, alkylcarbonyls, and nitros;
each R₆ is individually selected from the group consisting of —H, alkyls, allyls, and β-trimethylsilylethyl;
each R₈ is individually selected from the group consisting of alkyls, aralkyls, heterocyclyls, and heterocyclylalkyls;
each R₉ group is individually selected from the group consisting of —H, —F, and alkyls, wherein when two R₉ groups are geminal alkyl groups, said geminal alkyl groups may be cyclized to form a 3-6 membered ring; and
G is alkylene, N(R₆), O;
each Z is individually selected from the group consisting of —O— and —N(R₄)—;
each ring of formula (IA) optionally includes one or more of R₇, where R₇ is a noninterfering substituent individually selected from the group consisting of —H, alkyls, aryls, heterocyclyls, alkylaminos, arylaminos, cycloalkylaminos, heterocyclylaminos, hydroxys, alkoxys, aryloxys, alkylthios, arthylthios, cyanos, halogens, nitrilos, nitros, alkylsulfinyls, alkylsulfonyls, aminosulfonyls, and perfluoroalkyls;
except that:
when Q is Q-3 or Q-4, then the compound of formula (I) is not 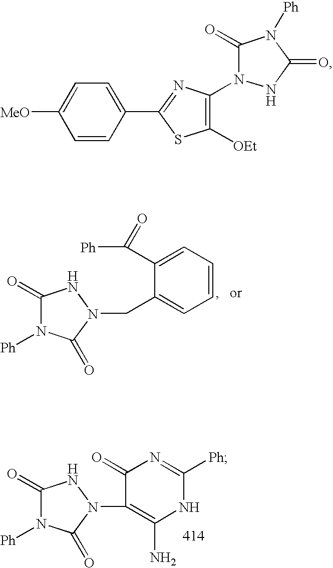
when Q is Q-7, q is 0, and R₅ and D are phenyl, then A is not phenyl, oxazolyl, pyridyl, pyrimidyl, pyrazolyl, or imidazolyl;
when Q is Q-7, R₅ is —OH, Y is —O—, —S—, or —CO—, m is 0, n is 0, p is 0, and A is phenyl, pyridyl, or thiazolyl, then D is not thienyl, thiazolyl, or phenyl;
when Q is Q-7, R₅ is —OH, m is 0, n is 0, p is 0, t is 0, and A is phenyl, pyridyl, or thiazolyl, then D is not thienyl, thiazolyl, or phenyl;
when Q is Q-7, then the compound of formula (I) is not 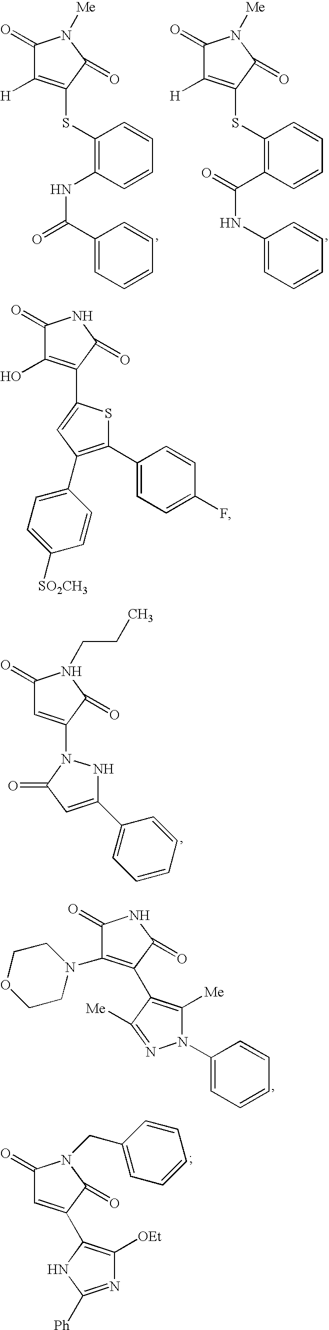 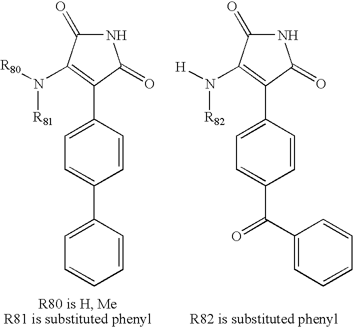
when Q is Q-8, then Y is not —CH₂O—;
when Q is Q-8, the compound of formula (I) is not 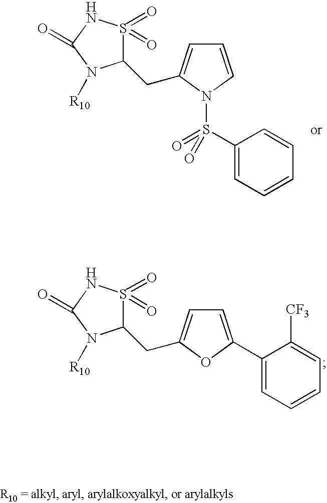
when Q is Q-9, then the compound of formula (I) is not 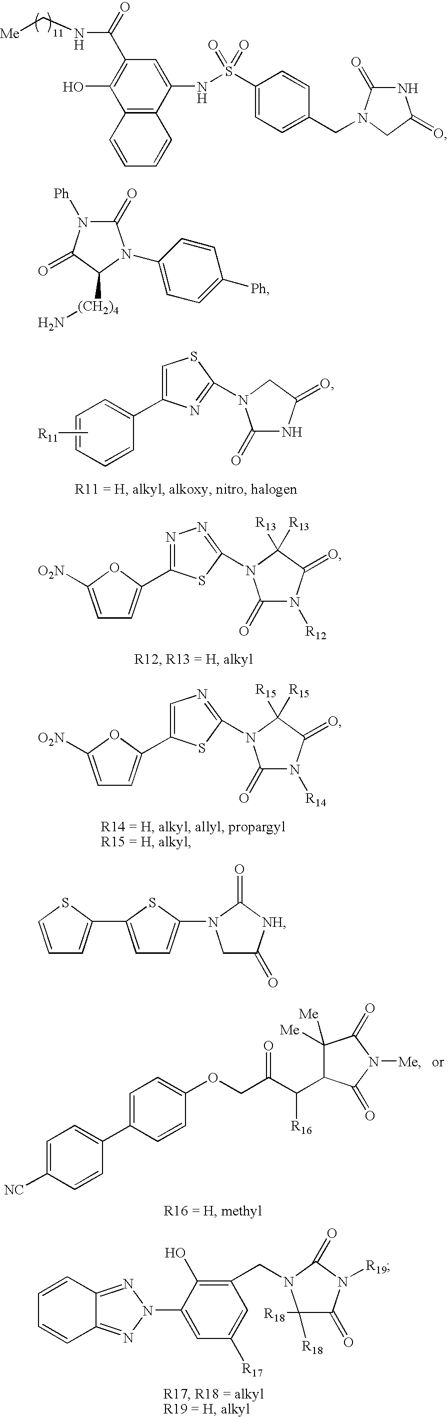
when Q is Q-10, t is 0, and E is phenyl, then any R₇ on E is not an o-alkoxy;
when Q is Q-10, then the compound of formula (I) is not 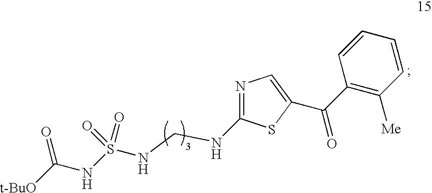
when Q is Q-11, t is 0, and E is phenyl, then any R₇ on E is not an o-alkoxy;
when Q is Q-11, then the compound of formula (I) is not 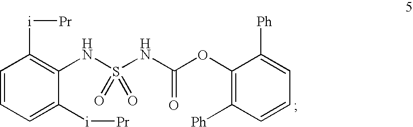
when Q is Q-15, then the compound of formula (I) is not 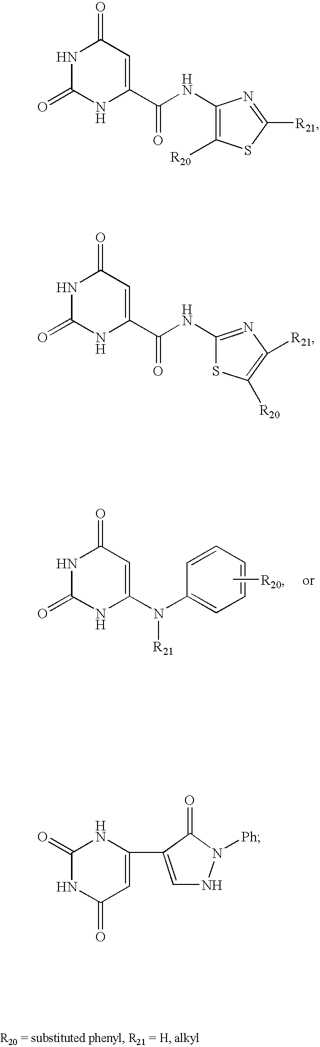
when Q is Q-16 and Y is —NH—, then 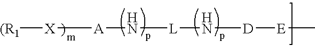
of formula (I) is not biphenyl;
when Q is Q-16 and Y is —S—, then 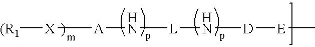
of formula (I) is not phenylsulfonylaminophenyl or phenylcarbonylaminophenyl;
when Q is Q-16 and Y is —SO₂NH—, then the compound of formula (I) is not 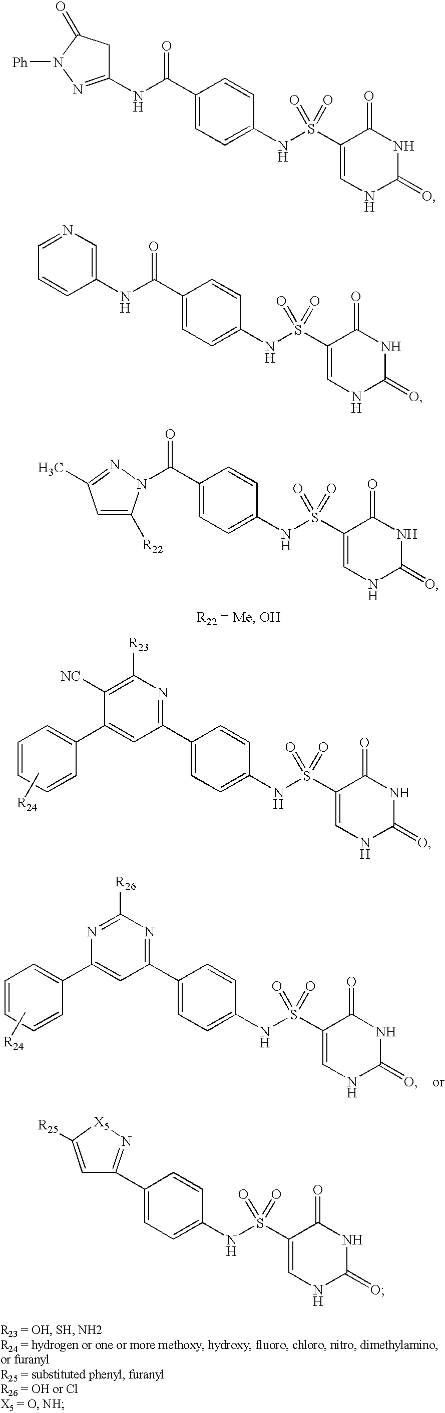
when Q is Q-16 and Y is —CONH—, then 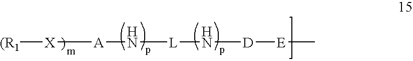
of formula (I) is not imidazophenyl;
when Q is Q-16 and Y is —CONH—, then the compound of formula (I) is not 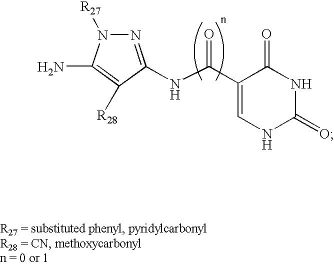
when Q is Q-16 and t is 0, then 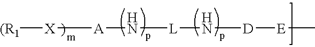
of formula (I) is not phenylcarbonylphenyl, pyrimidophenyl, phenylpyrimidyl, pyrimidyl, or N-pyrolyl;
when Q is Q-17, then the compound of formula (I) is not 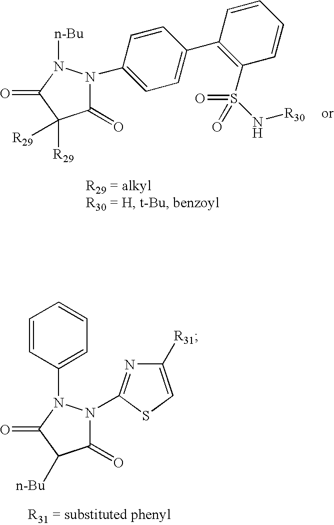
when Q is Q-21, then the compound of formula (I) is not 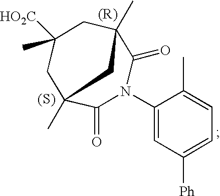
when Q is Q-22, then the compound of formula (I) is selected from the group consisting of 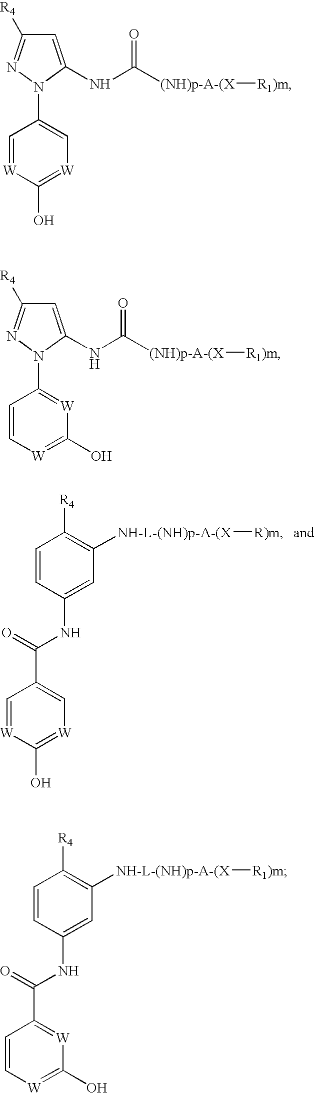
when Q is Q-22 and q is 0, then the compound of formula (I) is selected from the group consisting of 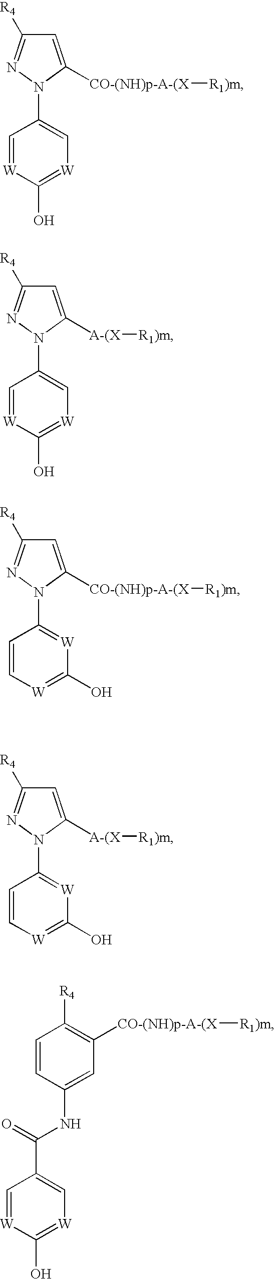 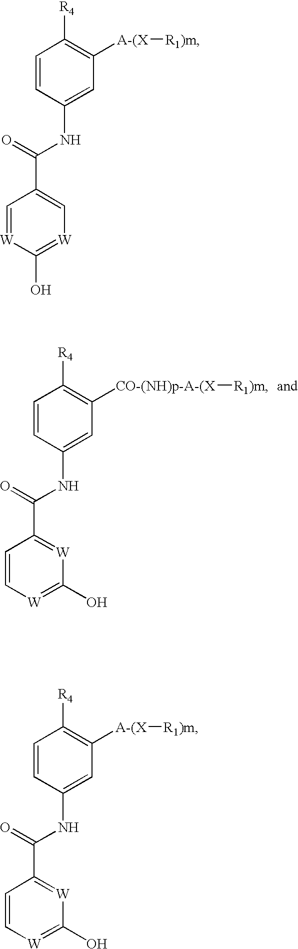
but excluding 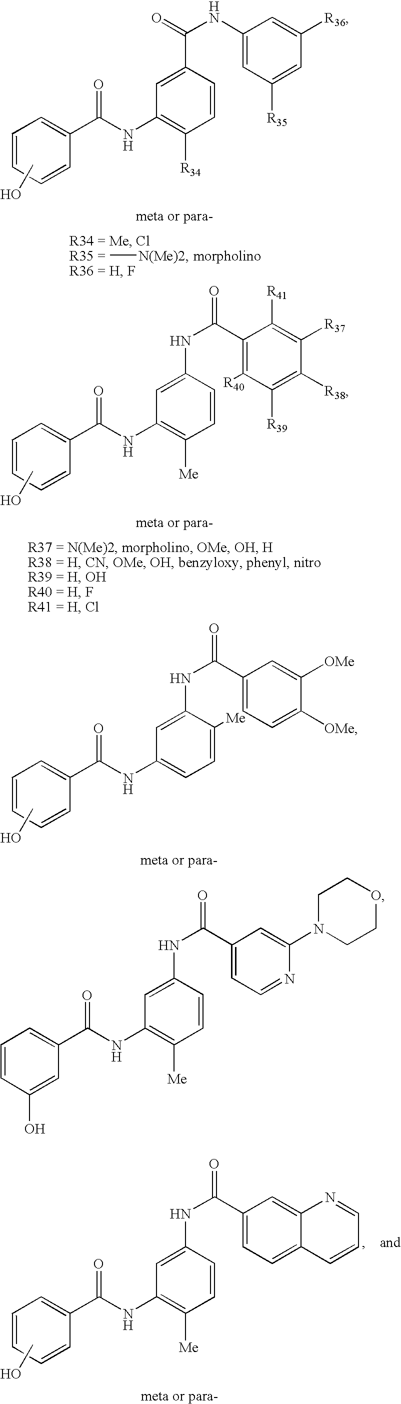 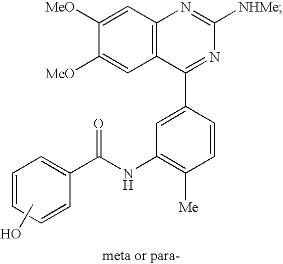
when Q is Q-23, then the compound of formula (I) is not 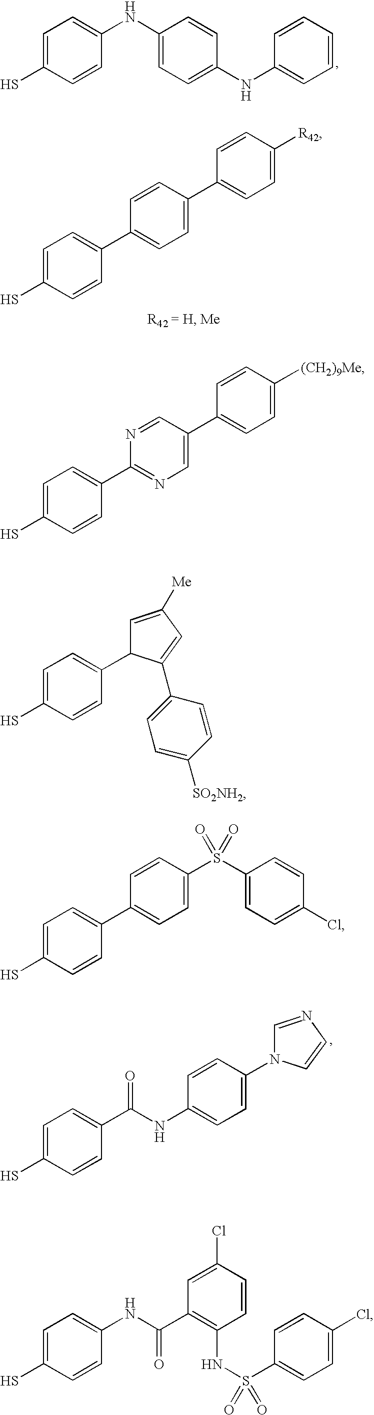 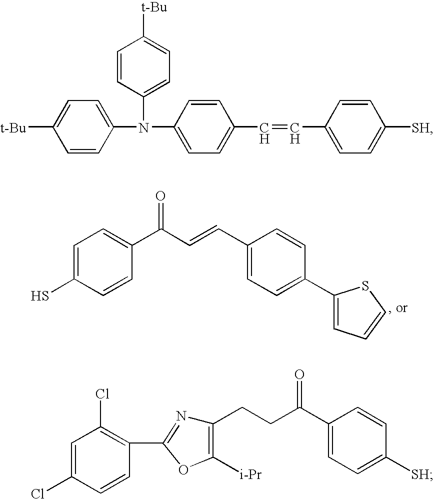
when Q is Q-24, Q-25, Q-26, or Q-31, then the compound of formula (I) is selected from the group consisting of 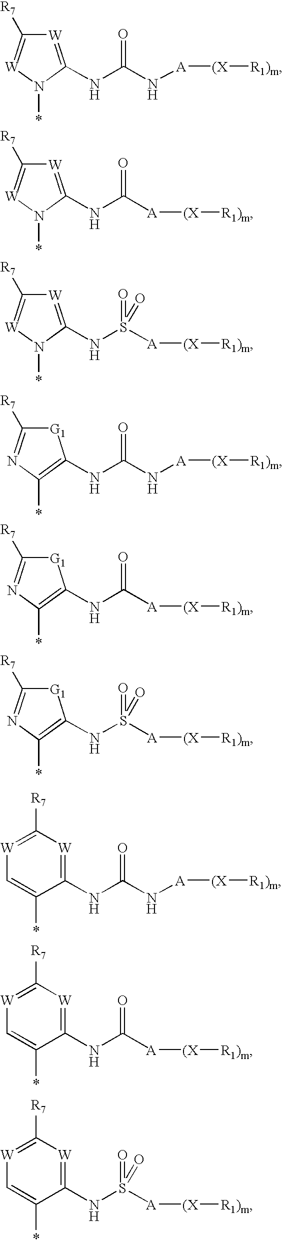 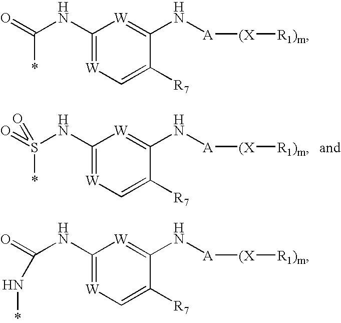
wherein each W is individually selected from the group consisting of —CH— and —N—;
each G₁ is individually selected from the group consisting of —O—, —S—, and —N(R₄)—; and
* denotes the point of attachment to Q-24, Q-25, Q-26, or Q-31 as follows: 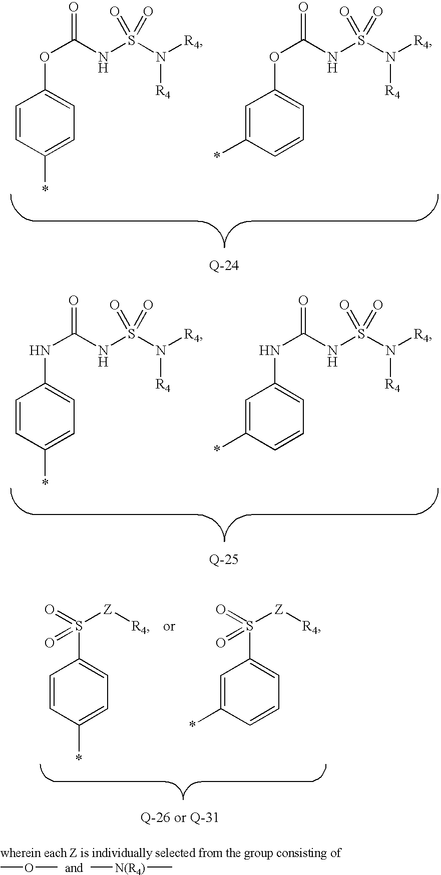
wherein each Z is individually selected from the group consisting of —O— and —N(R₄)—;
when Q is Q-31, then the compound of formula (I) is not 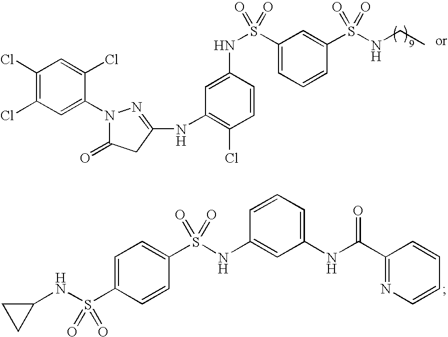
when Q is Q-28 or Q-29 and t is 0, then the compound of formula (I) is not 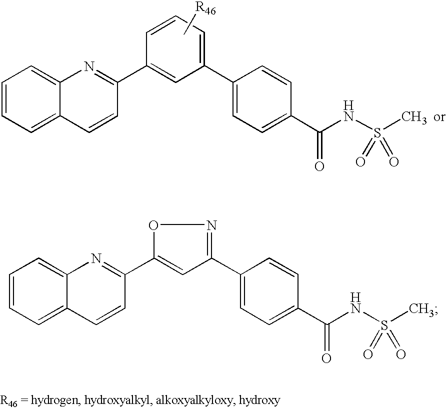
when Q is Q-28 or Q-29 and Y is an ether linkage, then the compound of formula (I) is not 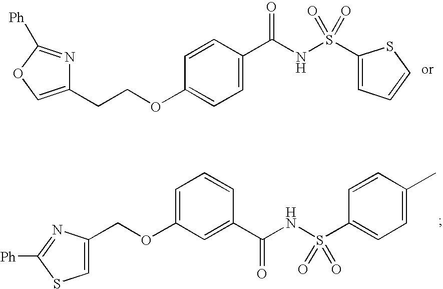
when Q is Q-28 or Q-29 and Y is —CONH—, then the compound of formula (I) is not 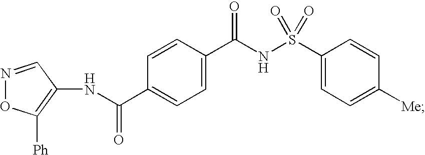
when Q is Q-32, then 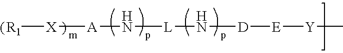
is not biphenyl, benzoxazolylphenyl, pyridylphenyl or bipyridyl;
when Q is Q-32, Y is —CONH—, q is 0, m is 0, and 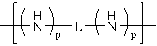
of formula (I) is —CONH—, then A is not phenyl;
when Q is Q-32, q is 0, m is 0, and 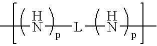
is —CONH—, then the compound of formula (I) is not 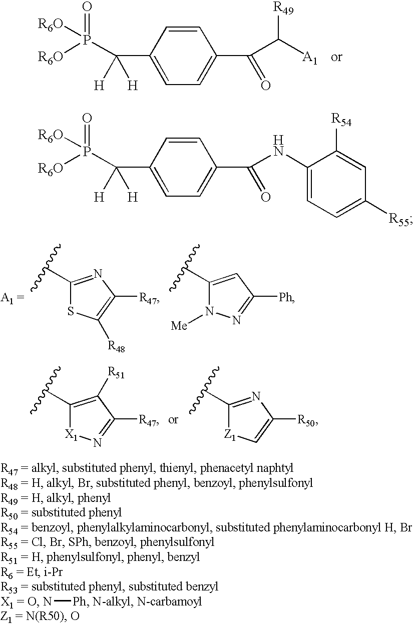
when Q is Q-32, D is thiazolyl, q is 0, t is 0, p is 0, n is 0, and m is 0, then A is not phenyl or 2-pyridone;
when Q is Q-32, D is oxazolyl or isoxazolyl, q is 0, t is 0, p is 0, n is 0, and m is 0, then A is not phenyl;
when Q is Q-32, D is pyrimidyl q is 0, t is 0, p is 0, n is 0, and m is 0, then A is not phenyl;
when Q is Q-32 and Y is an ether linkage, then 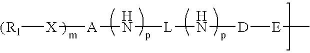
of formula (I) is not biphenyl or phenyloxazolyl;
when Q is Q-32 and Y is —CH═CH—, then 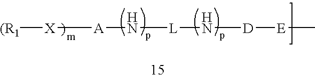
of formula (I) is not phenylaminophenyl;
when Q is Q-32, then the compound of formula (I) is not 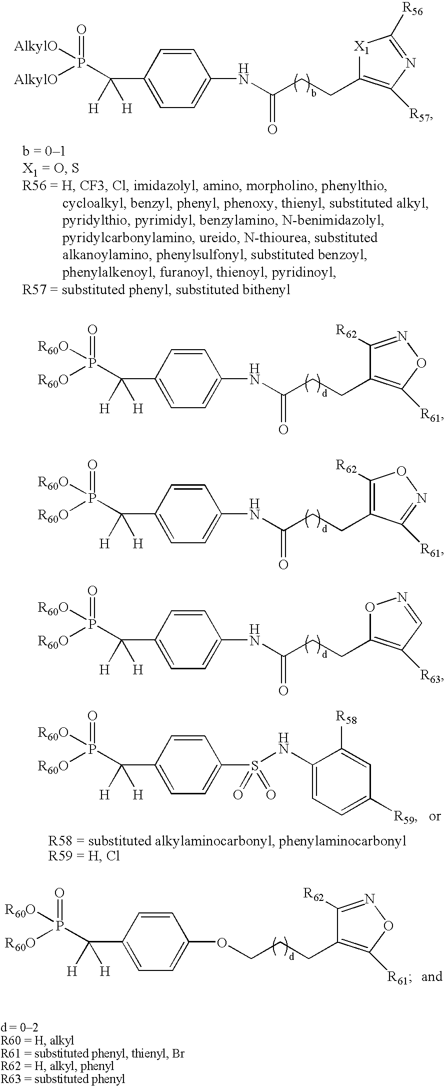
when Q is Q-35 as shown 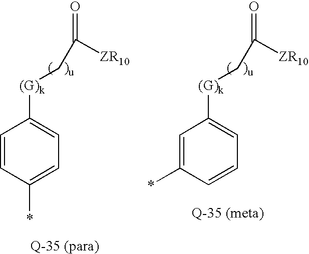
wherein G is selected from the group consisting of —O—, —S—, —NR₄—, and —CH₂—,
k is 0 or 1, and u is 1, 2, 3, or 4, then 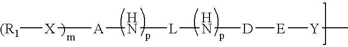
is selected from the group consisting of 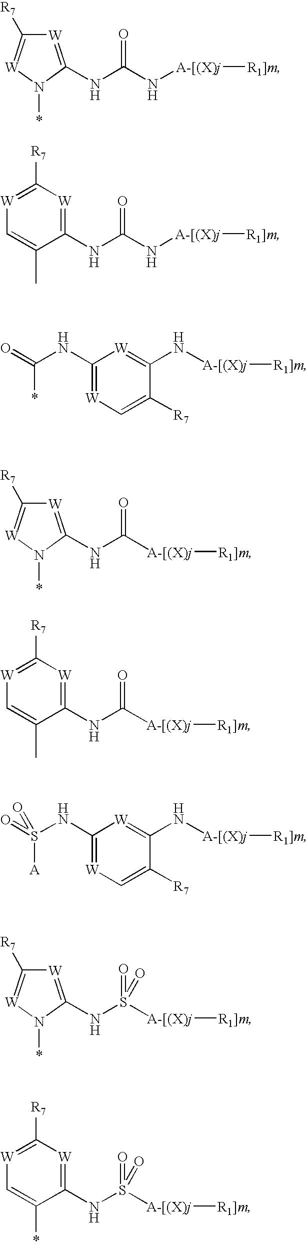 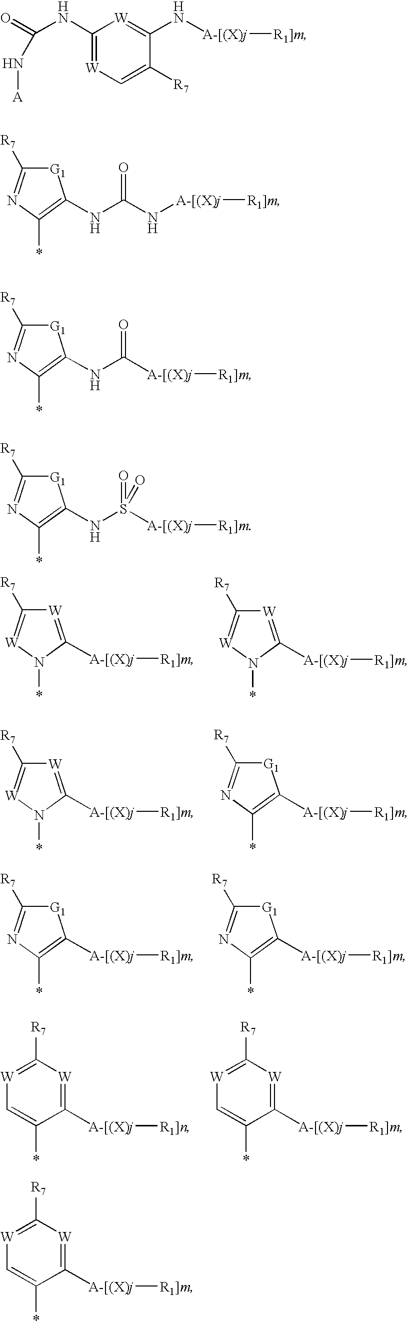 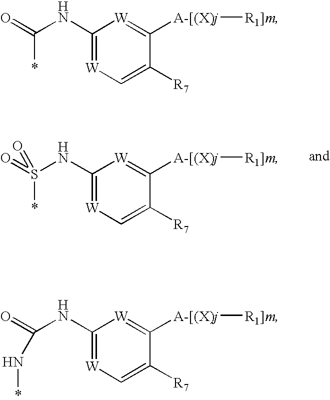
except that the compound of formula (I) is not

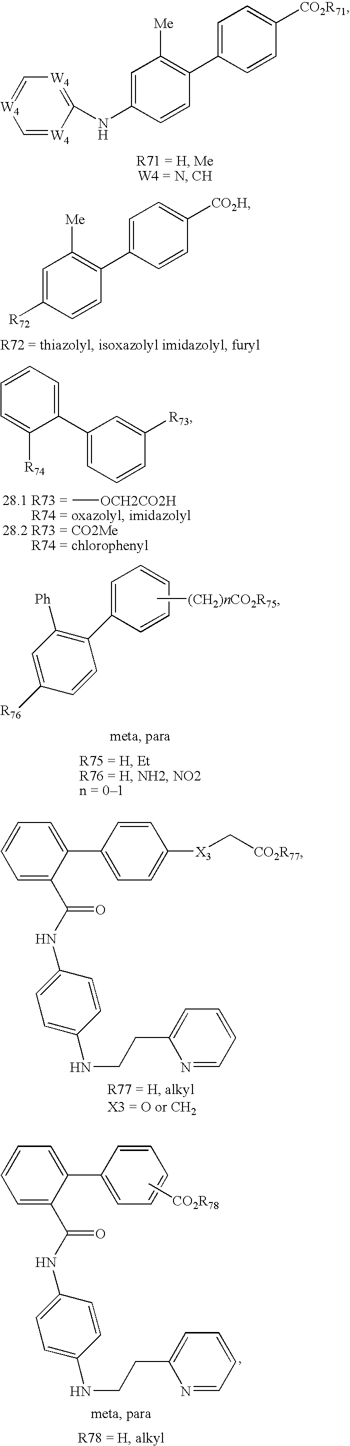

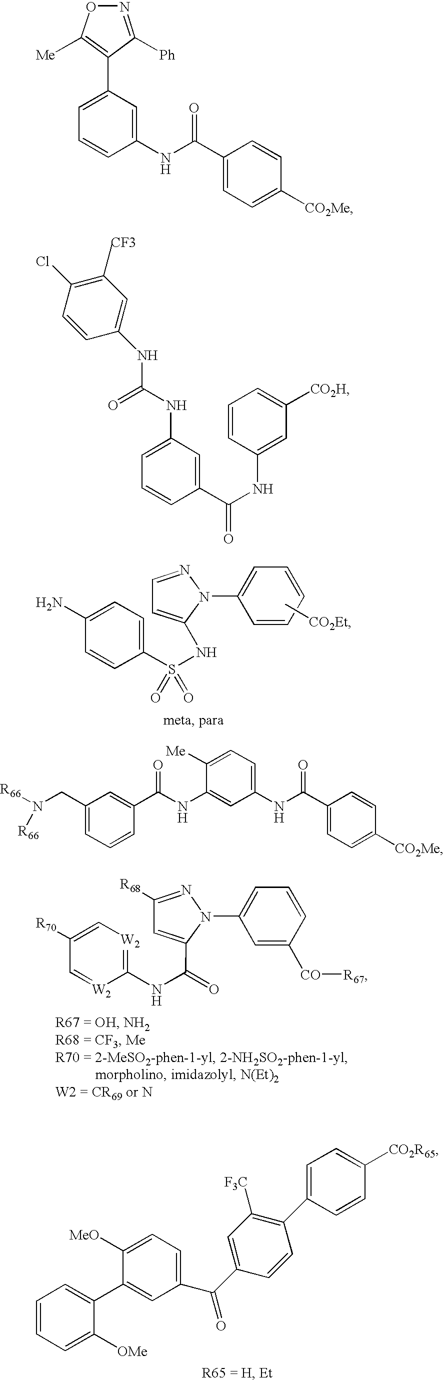

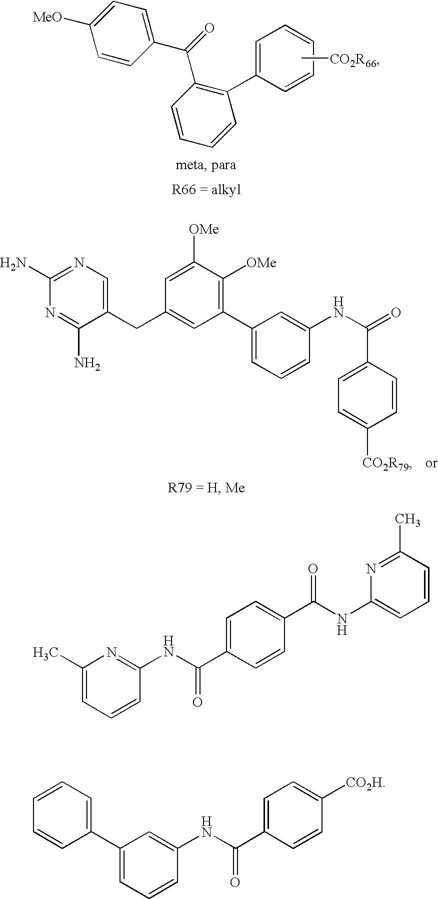

2. The compound of claim 1, wherein R₁ is selected from the group consisting of 6-5 fused heteroaryls, 6-5 fused heterocyclyls, 5-6 fused heteroaryls, and 5-6 fused heterocyclyls.

3. The compound of claim 2, where R₁ is selected from the group consisting of 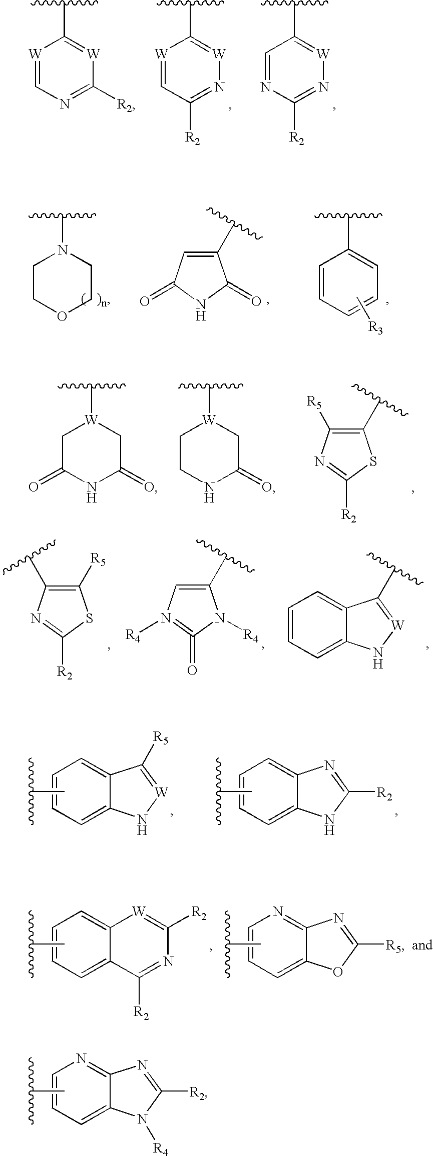
each R₂ is individually selected from the group consisting of —H, alkyls, aminos, alkylaminos, arylaminos, cycloalkylaminos, heterocyclylaminos, halogens, alkoxys, and hydroxys; and
each R₃ is individually selected from the group consisting of —H, alkyls, alkylaminos, arylaminos, cycloalkylaminos, heterocyclylaminos, alkoxys, hydroxys, cyanos, halogens, perfluoroalkyls, alkylsulfinyls, alkylsulfonyls, R₄NHSO₂—, and —NHSO₂R₄.

4. The compound of claim 1, wherein A is selected from the group consisting of aromatic, monocycloheterocyclic, and bicycloheterocyclic rings; and most preferably phenyl, naphthyl, pyridyl, pyrimidyl, thienyl, furyl, pyrrolyl, thiazolyl, isothiazolyl, oxaxolyl, isoxazolyl, imidazolyl, oxadiazolyl, thiadiazolyl, indolyl, indazolyl, benzimidazolyl, benzotriazolyl, isoquinolyl, quinolyl, benzothiazolyl, benzofuranyl, benzothienyl, pyrazolylpyrimidinyl, imidazopyrimidinyl, purinyl, and 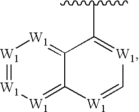
where each W₁ is individually selected from the group consisting of —CH— and —N—.

5. A method of modulating the activation state of p38 α-kinase comprising the step of contacting said kinase with a molecule having the formula 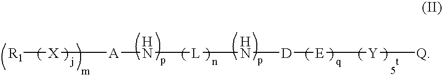
wherein:
R¹ is selected from the group consisting of aryls and heteroaryls;
each X and Y is individually selected from the group consisting of —O—, —S—, —NR₆—, —NR₆SO₂—, —NRrCO—, alkynyls, alkenyls, alkylenes, —O(CH₂)ₕ—, and —NR₆(CH₂)ₕ—, where each h is individually selected from the group consisting of 1, 2, 3, or 4, and where for each of alkylenes, —O(CH₂)ₕ—, and —NR₆(CH₂)ₕ—, one of the methylene groups present therein may be optionally double-bonded to a side-chain oxo group except that where —O(CH₂)ₕ— the introduction of the side-chain oxo group does not form an ester moiety;
A is selected from the group consisting of aromatic, monocycloheterocyclic, and bicycloheterocyclic rings;
D is phenyl or a five- or six-membered heterocyclic ring selected from the group consisting of pyrazolyl, pyrrolyl, imidazolyl, oxazolyl, thiazolyl, furyl, oxadiazolyl, thiadiazolyl, thienyl, pyridyl, and pyrimidyl;
E is selected from the group consisting of phenyl, pyridinyl, and pyrimidinyl;
L is selected from the group consisting of —C(O)— and —S(O)₂—;
j is 0 or 1;
m is 0 or 1;
n is 0 or 1;
p is 0 or 1;
q is 0 or 1;
t is 0 or 1;
Q is selected from the group consisting of 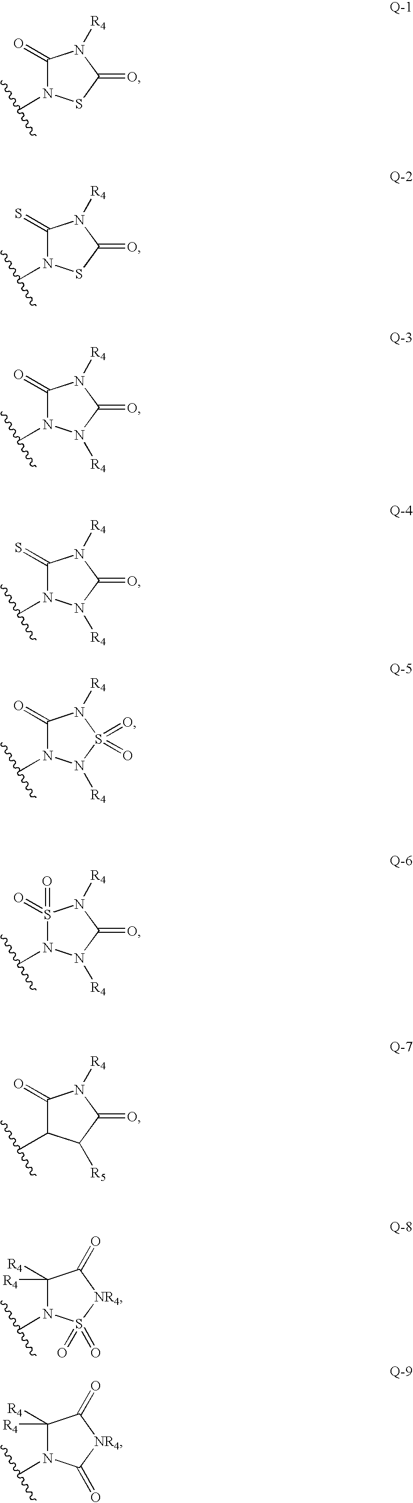 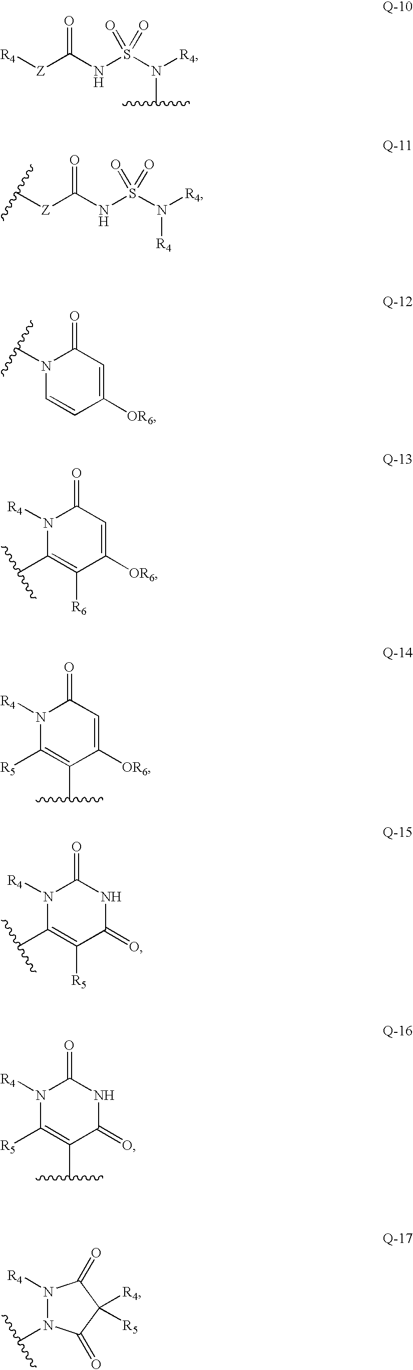 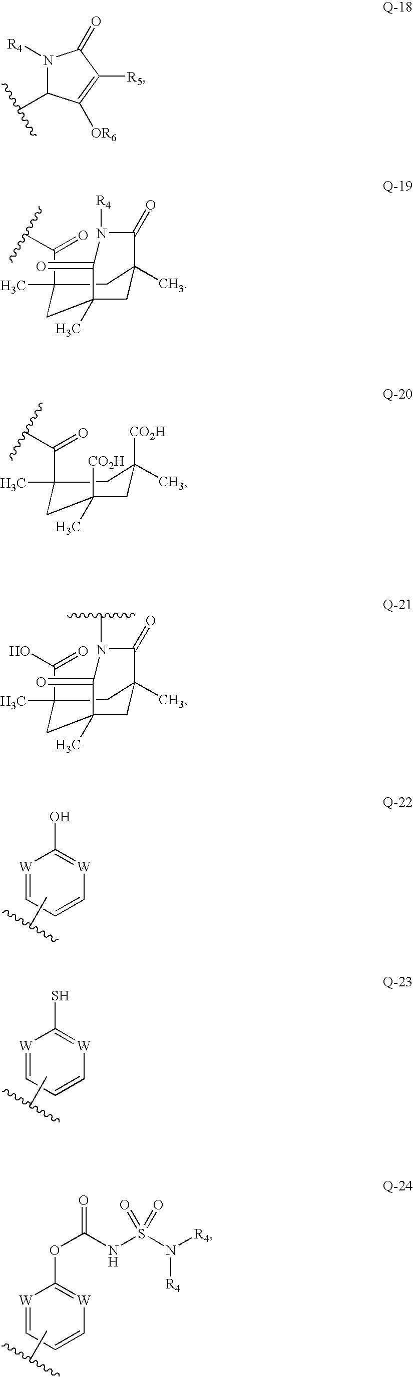 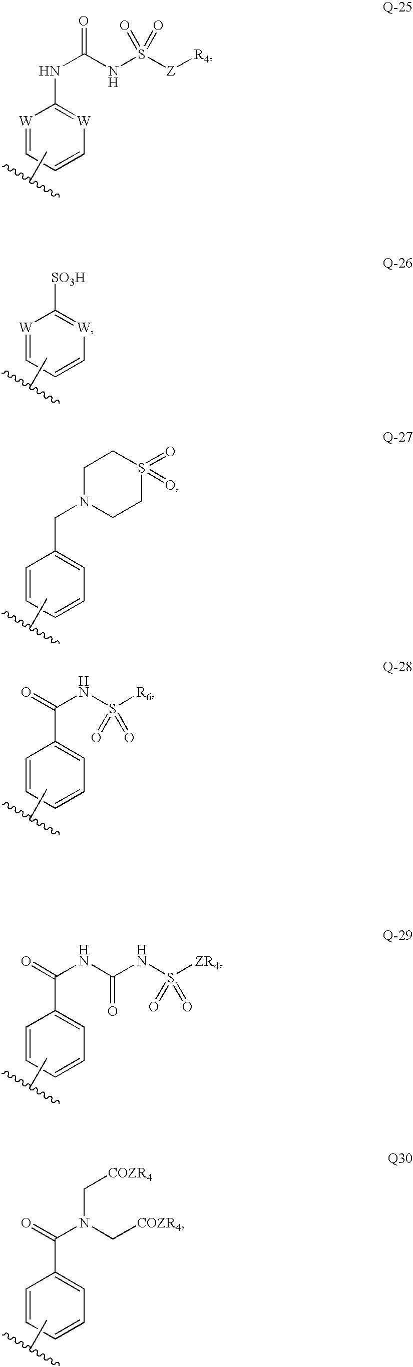 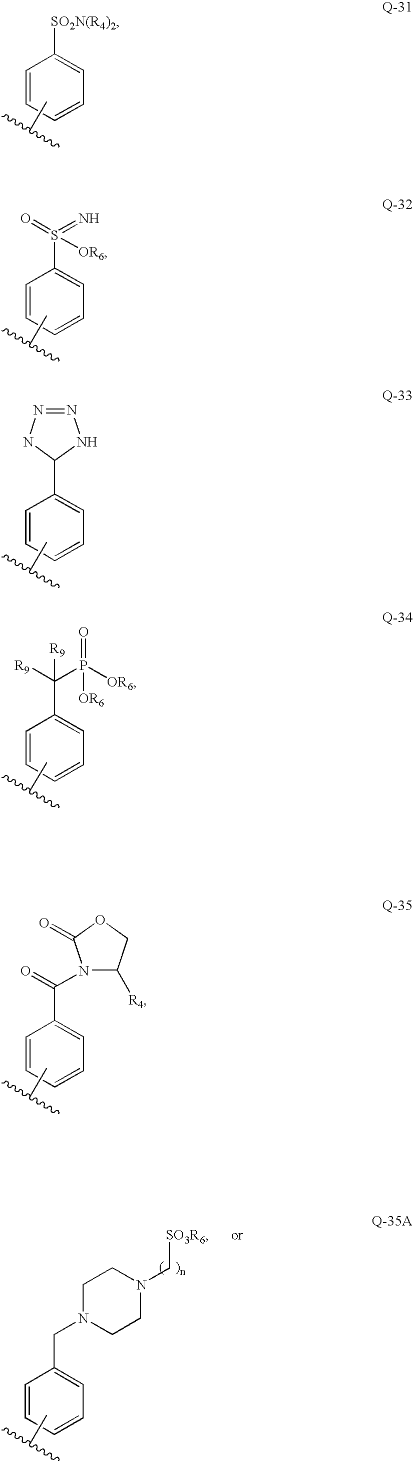 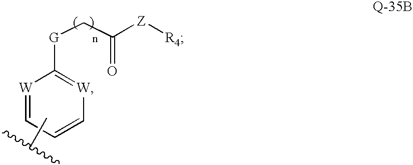
each R₄ group is individually selected from the group consisting of —H, alkyls, aminoalkyls, alkoxyalkyls, aryls, aralkyls, heterocyclyls, and heterocyclylalkyls except when the R₄ substituent places a heteroatom on an alpha-carbon directly attached to a ring nitrogen on Q;
when two R₄ groups are bonded with the same atom, the two R₄ groups optionally form an alicyclic or heterocyclic 4-7 membered ring;
each R₅ is individually selected from the group consisting of —H, alkyls, aryls, heterocyclyls, alkylaminos, arylaminos, cycloalkylaminos, heterocyclylaminos, hydroxys, alkoxys, aryloxys, alkylthios, arylthios, cyanos, halogens, perfluoroalkyls, alkylcarbonyls, and nitros;
each R₆ is individually selected from the group consisting of —H, alkyls, allyls, and β-trimethylsilylethyl;
each R₈ is individually selected from the group consisting of alkyls, aralkyls, heterocyclyls, and heterocyclylalkyls;
each R₉ group is individually selected from the group consisting of —H, —F, and alkyls, wherein when two R₉ groups are geminal alkyl groups, said geminal alkyl groups may be cyclized to form a 3-6 membered ring;
G is alkylene, N(R₆), O;
each Z is individually selected from the group consisting of —O— and —N(R₄)—; and
each ring of formula (II) optionally includes one or more of R₇, where R₇ is a noninterfering substituent individually selected from the group consisting of —H, alkyls, aryls, heterocyclyls, alkylaminos, arylaminos, cycloalkylaminos, heterocyclylaminos, hydroxys, alkoxys, aryloxys, alkylthios, arthylthios, cyanos, halogens, nitrilos, nitros, alkylsulfinyls, alkylsulfonyls, aminosulfonyls, and perfluoroalkyls,
and thereby causing modulation of said activation state.

6. The method of claim 5, said contacting step occurring at the region of a switch control pocket of said kinase.

7. The method of claim 6, said switch control pocket of said kinase comprising an amino acid residue sequence operable for binding to said Formula (II) molecule.

8. The method of claim 6, said switch control pocket selected from the group consisting of simple, composite and combined switch control pockets.

9. The method of claim 8, said region being selected from the group consisting of the α-C helix, the α-D helix, the catalytic loop, the switch control ligand sequence, the C-lobe residues, the glycine rich loop residues, and combinations thereof.

10. The method of claim 9, said α-C helix including SEQ ID NO. 2.

11. The method of claim 9, said catalytic loop including SEQ ID NO. 3.

12. The method of claim 9, said switch control ligand sequence being selected from the group consisting of SEQ ID NO. 4, SEQ ID NO. 5, and combinations thereof.

13. The method of claim 9, said C-lobe residues including SEQ ID NO. 6.

14. The method of claim 9, said glycine rich loop residues including SEQ ID NO. 7.

15. The method of claim 5, said kinase selected from the group consisting of the consensus wild type sequence and disease polymorphs thereof.

16. The method of claim 5, said activation state being selected from the group consisting of the upregulated and downregulated states.

17. The method of claim 5, said molecule being an antagonist of the on switch control pocket for said kinase.

18. The method of claim 5, said molecule being an agonist of the off switch control pocket for said kinase.

19. The method of claim 5, said method including the step of administering said molecule to an individual undergoing treatment for a condition selected from the group consisting of human inflammation, rheumatoid arthritis, rheumatoid spondylitis, ostero-arthritis, asthma, gouty arthritis, sepsis, septic shock, endotoxic shock, Gram-negative sepsis, toxic shock syndrome, adult respiratory distress syndrome, stroke, reperfusion injury, neural trauma, neural ischemia, psoriasis, restenosis, chronic pulmonary inflammatory disease, bone resorptive diseases, graft-versus-host reaction, Chron's disease, ulcerative colitis, inflammatory bowel disease, pyresis, and combinations thereof.

20. The method of claim 19, said molecule being administered by a method selected from the group consisting of oral, parenteral, inhalation, and subcutaneous.

21. The method of claim 5, said molecule having the structure of the compound of claim 1.

22. An adduct comprising a molecule binding with a kinase, said molecule having the formula 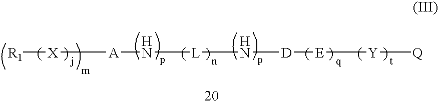
wherein:
R¹ is selected from the group consisting of aryls and heteroaryls;
each X and Y is individually selected from the group consisting of —O—, —S—, —NR₆—, —NR₆SO₂—, —NR₆CO—, alkynyls, alkenyls, alkylenes, —O(CH₂)ₕ—, and —NR₆(CH₂)ₕ—, where each h is individually selected from the group consisting of 1, 2, 3, or 4, and where for each of alkylenes, —O(CH₂)ₕ—, and —NR₆(CH₂)ₕ—, one of the methylene groups present therein may be optionally double-bonded to a side-chain oxo group except that where —O(CH₂)ₕ— the introduction of the side-chain oxo group does not form an ester moiety;
A is selected from the group consisting of aromatic, monocycloheterocyclic, and bicycloheterocyclic rings;
D is phenyl or a five- or six-membered heterocyclic ring selected from the group consisting of pyrazolyl, pyrrolyl, imidazolyl, oxazolyl, thiazolyl, furyl, oxadiazolyl, thiadiazolyl, thienyl, pyridyl, and pyrimidyl;
E is selected from the group consisting of phenyl, pyridinyl, and pyrimidinyl;
L is selected from the group consisting of —C(O)— and —S(O)₂—;
j is 0 or 1;
m is 0 or 1;
n is 0 or 1;
p is 0 or 1;
q is 0 or 1;
t is 0 or 1;
Q is selected from the group consisting of 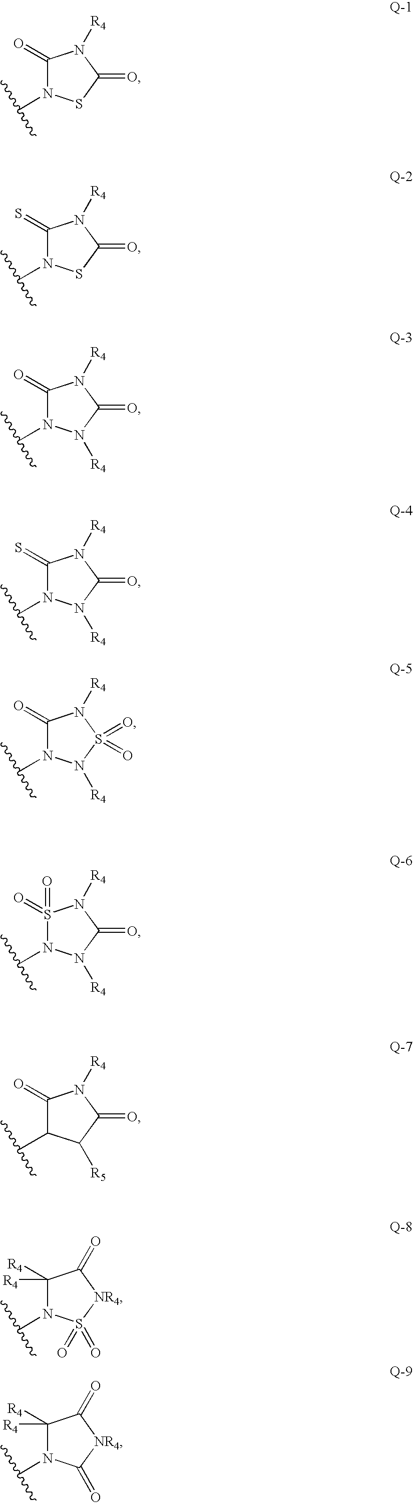 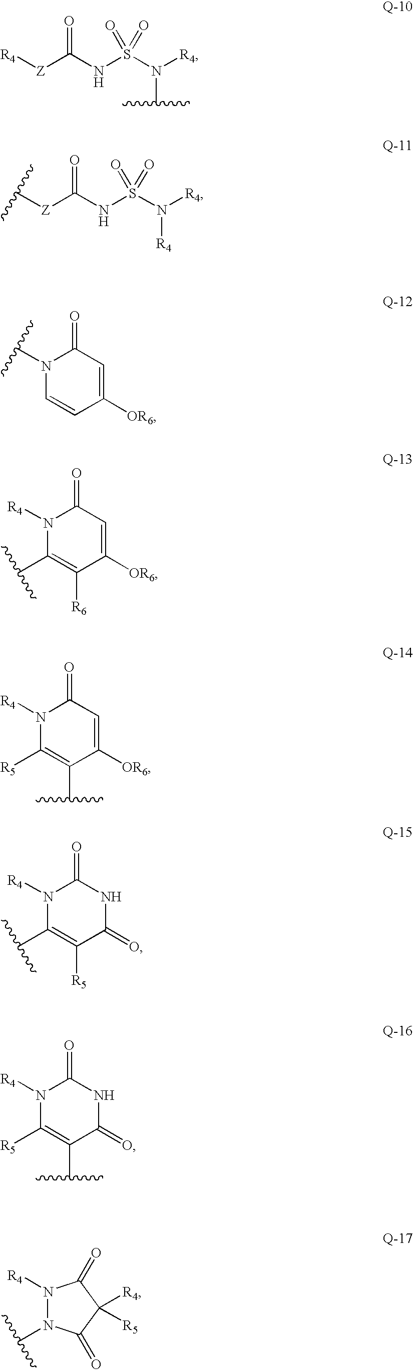 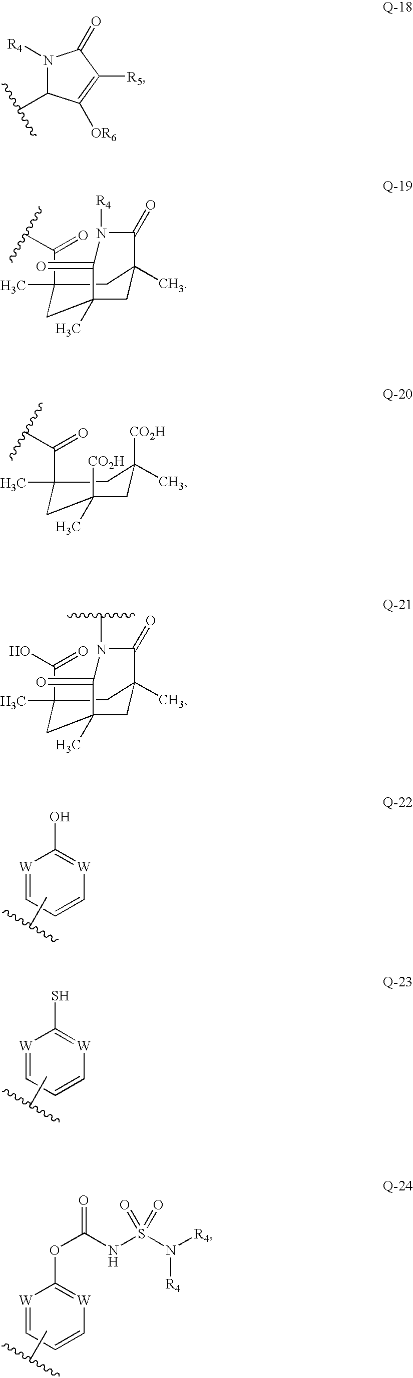 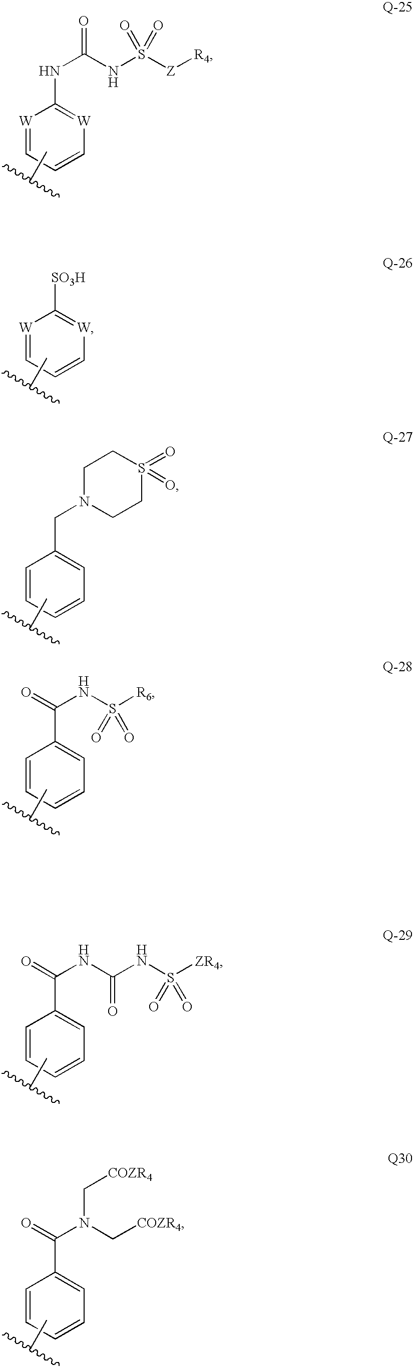 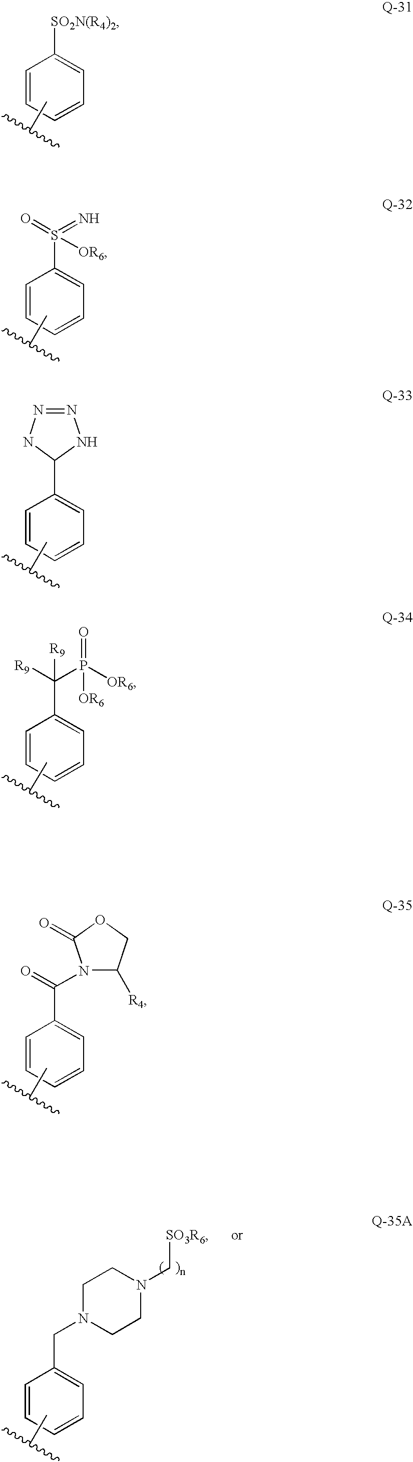 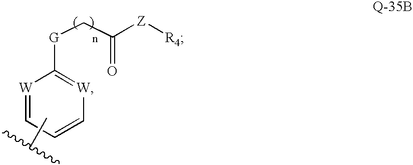
each R₄ group is individually selected from the group consisting of —H, alkyls, aminoalkyls, alkoxyalkyls, aryls, aralkyls, heterocyclyls, and heterocyclylalkyls except when the R₄ substituent places a heteroatom on an alpha-carbon directly attached to a ring nitrogen on Q;
when two R₄ groups are bonded with the same atom, the two R₄ groups optionally form an alicyclic or heterocyclic 4-7 membered ring;
each R₅ is individually selected from the group consisting of —H, alkyls, aryls, heterocyclyls, alkylaminos, arylaminos, cycloalkylaminos, heterocyclylaminos, hydroxys, alkoxys, aryloxys, alkylthios, arylthios, cyanos, halogens, perfluoroalkyls, alkylcarbonyls, and nitros;
each R₆ is individually selected from the group consisting of —H, alkyls, allyls, and β-trimethylsilylethyl;
each R₈ is individually selected from the group consisting of alkyls, aralkyls, heterocyclyls, and heterocyclylalkyls;
each R₉ group is individually selected from the group consisting of —H, —F, and alkyls, wherein when two R₉ groups are geminal alkyl groups, said geminal alkyl groups may be cyclized to form a 3-6 membered ring;
G is alkylene, N(R₆), O;
each Z is individually selected from the group consisting of —O— and —N(R₄)—; and
each ring of formula (III) optionally includes one or more of R₇, where R₇ is a noninterfering substituent individually selected from the group consisting of —H, alkyls, aryls, heterocyclyls, alkylaminos, arylaminos, cycloalkylaminos, heterocyclylaminos, hydroxys, alkoxys, aryloxys, alkylthios, arthylthios, cyanos, halogens, nitrilos, nitros, alkylsulfinyls, alkylsulfonyls, aminosulfonyls, and perfluoroalkyls.

23. The adduct of claim 22, said molecule binding at the region of a switch control pocket of said kinase.

24. The adduct of claim 23, said switch control pocket of said kinase comprising an amino acid residue sequence operable for binding to said Formula (III) molecule.

25. The adduct of claim 23, said switch control pocket selected from the group consisting of simple, composite and combined switch control pockets.

26. The adduct of claim 25, said region being selected from the group consisting of the α-C helix, the α-D helix, the catalytic loop, the switch control ligand sequence, the C-terminal residues, the glycine rich loop residues, and combinations thereof.

27. The adduct of claim 26, said α-C helix including SEQ ID NO. 2.

28. The adduct of claim 26, said catalytic loop including SEQ ID NO. 3.

29. The adduct of claim 26, said switch control ligand sequence being selected from the group consisting of SEQ ID NO. 4, SEQ ID NO. 5, and combinations thereof.

30. The adduct of claim 26, said C-lobe residues selected from SEQ ID NO. 6.

31. The adduct of claim 26, said glycine rich loop residues taken from SEQ ID NO. 7.

32. The adduct of claim 22, said kinase selected from the group consisting of the consensus wild type sequence and disease polymorphs thereof.

33. The adduct of claim 22, said molecule having the structure of the compound of claim 1.

34. A compound having the formula
A-T-(L)ₙ-(NH)ₚ-D-(E)_{q}-(Y)ₜ-Q (IB)
wherein:
Y is selected from the group consisting of —O—, —S—, —NR₆—, —NR₆SO₂—, —NR₆CO—, alkynyls, alkenyls, alkylenes, —O(CH₂)ₕ—, and —NR₆(CH₂)ₕ—, where each h is individually selected from the group consisting of 1, 2, 3, or 4, and where for each of alkylenes, —O(CH₂)ₕ—, and —NR₆(CH₂)ₕ—, one of the methylene groups present therein may be optionally double-bonded to a side-chain oxo group except that where —O(CH₂)ₕ— the introduction of the side-chain oxo group does not form an ester moiety;
A is selected from the group consisting of aromatic, monocycloheterocyclic, and bicycloheterocyclic rings; and most preferably phenyl, naphthyl, pyridyl, pyrimidyl, thienyl, furyl, pyrrolyl, thiazolyl, isothiazolyl, oxaxolyl, isoxazolyl, imidazolyl, oxadiazolyl, thiadiazolyl, indolyl, indazolyl, benzimidazolyl, benzotriazolyl, isoquinolyl, quinolyl, benzothiazolyl, benzofuranyl, benzothienyl, pyrazolylpyrimidinyl, imidazopyrimidinyl, purinyl, and 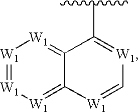
where each W1 is individually selected form the group consisting of —CH— and —N—.
D is phenyl or a five- or six-membered heterocyclic ring selected from the group consisting of pyrazolyl, pyrrolyl, imidazolyl, oxazolyl, thiazolyl, furyl, oxadiazolyl, thiadiazolyl, thienyl, pyridyl, and pyrimidyl;
E is selected from the group consisting of phenyl, pyridinyl, and pyrimidinyl;
L is selected from the group consisting of —C(O)— and —S(O)₂—;
T is NR₆, O, alkylene, —O(CH₂)ₕ—, or —NR₆(CH₂)ₕ—, where each h is individually selected from the group consisting of 1, 2, 3, or 4, or T is absent wherein A is directly bonded to -L)ₙ(NH)ₚ-D-(E)_{q}-(Y)ₜ-Q;
n is 0 or 1;
p is 0 or 1;
q is 0 or 1;
t is 0 or 1;
v is 1, 2, or 3;
x is 1 or 2;
Q is selected from the group consisting of formulae Q36-Q59, inclusive,
each R₄ group is individually selected from the group consisting of —H, alkyls, aminoalkyls, alkoxyalkyls, aryls, aralkyls, heterocyclyls, and heterocyclylalkyls except when the R₄ substituent places a heteroatom on an alpha-carbon directly attached to a ring nitrogen on Q;
when two R₄ groups are bonded with the same atom, the two R₄ groups optionally form an alicyclic or heterocyclic 4-7 membered ring;
each R₆ is individually selected from the group consisting of —H, alkyls, allyls, and -trimethylsilylethyl;
each R₈ is individually selected from the group consisting of alkyls, phenyl, naphthyl, aralkyls, heterocyclyls, and heterocyclylalkyls;
each R₉ group is individually selected from the group consisting of —H, —F, and alkyls, wherein when two R₉ groups are geminal alkyl groups, said geminal alkyl groups may be cyclized to form a 3-6 membered ring;
each R_{9′} group is individually selected from the group consisting of —F, and alkyls, wherein when two R₉ groups are geminal alkyl groups, said geminal alkyl groups may be cyclized to form a 3-6 membered ring;
each R₁₀ is alkyl or perfluoroalkyl;
G is alkylene, N(R₆), O;
each Z is individually selected from the group consisting of —O— and —N(R₄)—;
and each ring of formula (I) optionally includes one or more of R₇, where R_{7′} is a substituent individually selected from the group consisting of —H, alkyls, aryls, heterocyclyls, alkylaminos, arylaminos, cycloalkylaminos, heterocyclylaminos, hydroxys, alkoxys, perfluoroalkoxys, aryloxys, alkylthios, arthylthios, cyanos, halogens, nitrilos, nitros, alkylsulfinyls, alkylsulfonyls, aminosulfonyls, perfluoroalkyls; aminooxaloylamino; alkylaminooxaloylamino; dialkylaminooxaloylamino; morpholinooxaloylamino; piperazinooxaloylamino; alkoxycarbonylamino; heterocyclyloxycarbonylamino; heterocyclylalkyloxycarbonylamino; heterocyclylcarbonylamino; heterocyclylalkylcarbonylamino; aminoalkyloxycarbonylamino; alkylaminoalkyloxycarbonylamino; or dialkylaminoalkyloxycarbonylamino, said Q36-Q59 groups being

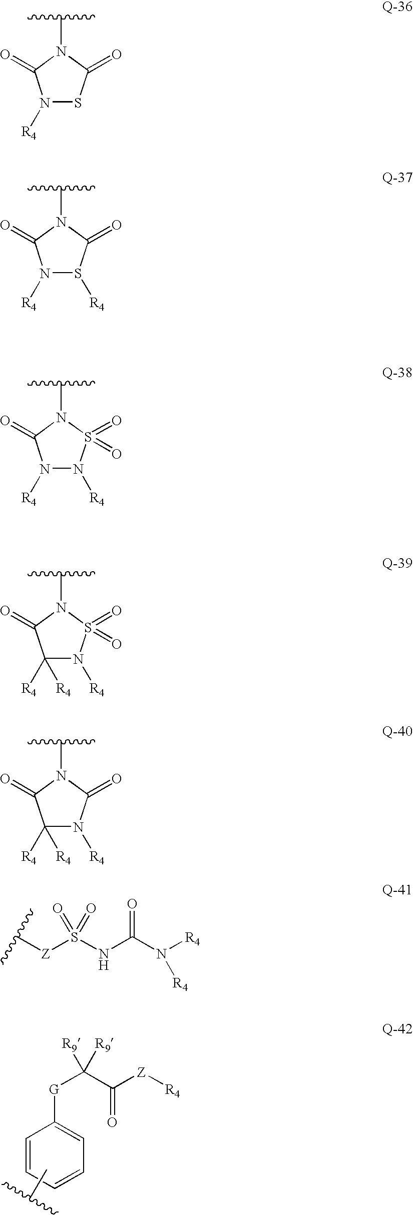

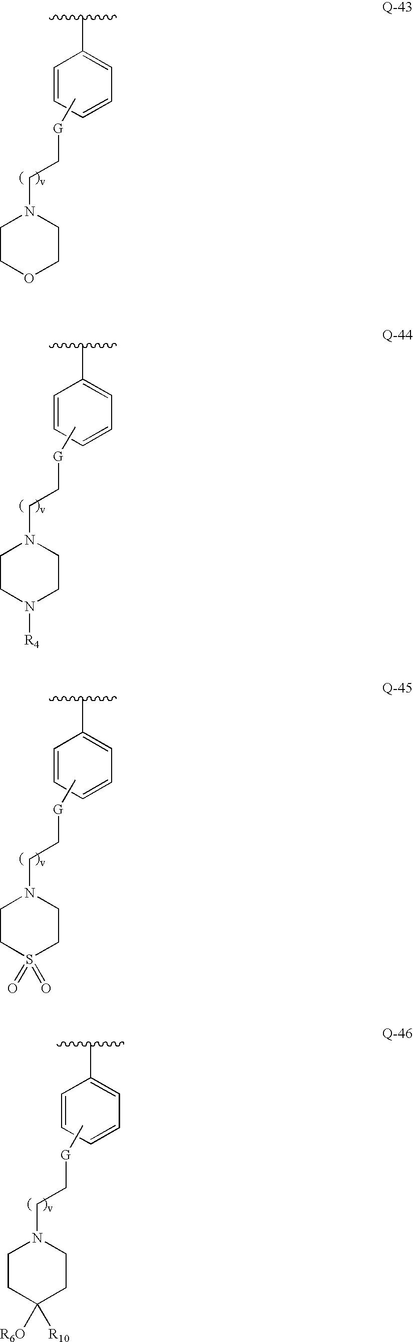

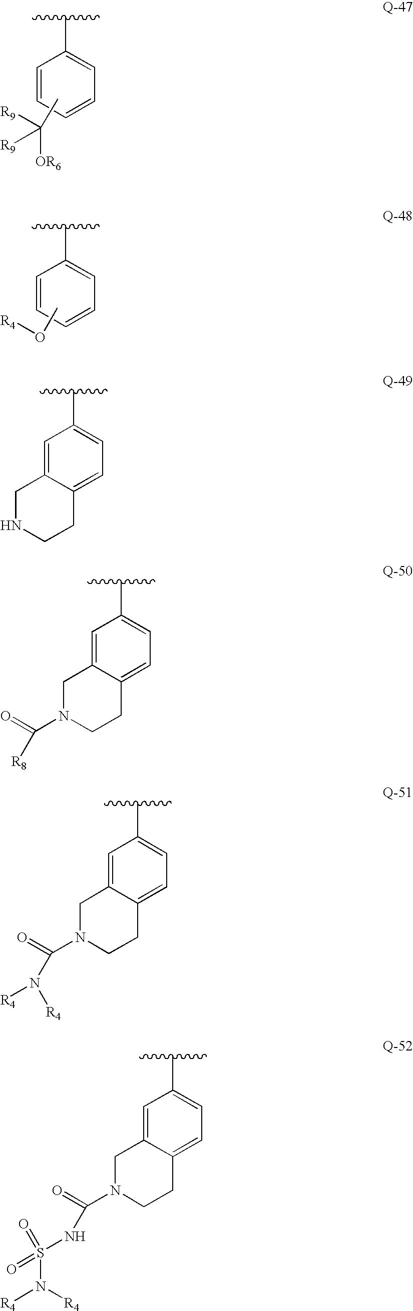

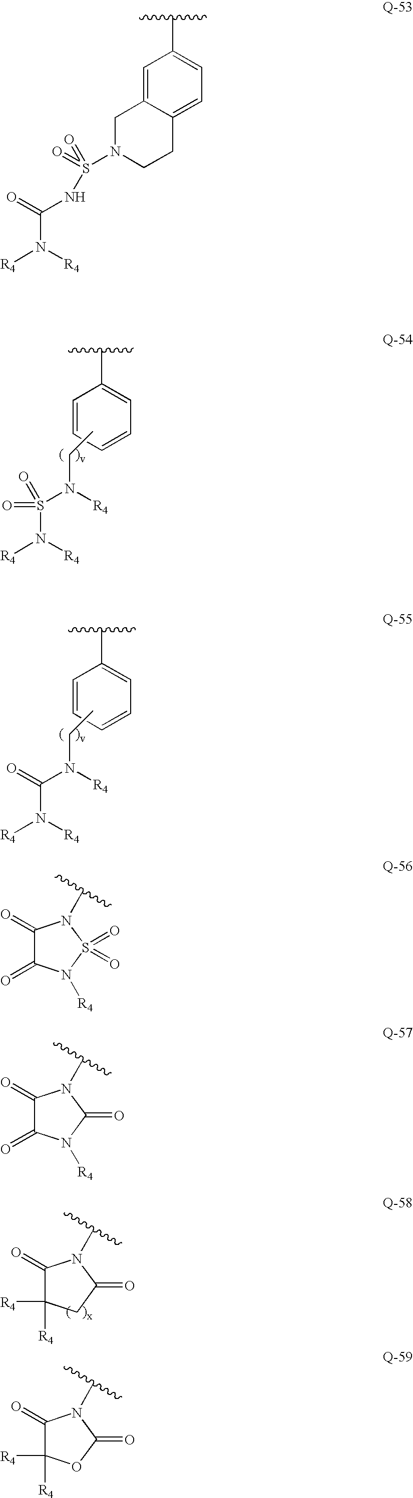

35. A method of modulating the activation state of p38 α-kinase comprising the step of contacting said kinase with a molecule having the formula of claim 34.

36. The method of claim 35, said contacting step occurring at the region of a switch control pocket of said kinase.

37. The method of claim 36, said switch control pocket of said kinase comprising an amino acid residue sequence operable for binding to said Formula (IB) molecule.

38. The method of claim 36, said switch control pocket selected from the group consisting of simple, composite and combined switch control pockets.

39. The method of claim 38, said region being selected from the group consisting of the α-C helix, the α-D helix, the catalytic loop, the switch control ligand sequence, the C-lobe residues, the glycine rich loop residues, and combinations thereof.

40. The method of claim 39, said α-C helix including SEQ ID NO. 2.

41. The method of claim 39, said catalytic loop including SEQ ID NO. 3.

42. The method of claim 39, said switch control ligand sequence being selected from the group consisting of SEQ ID NO. 4, SEQ ID NO. 5, and combinations thereof.

43. The method of claim 39, said C-lobe residues including SEQ ID NO. 6.

44. The method of claim 39, said glycine rich loop residues including SEQ ID NO. 7.

45. The method of claim 35, said kinase selected from the group consisting of the consensus wild type sequence and disease polymorphs thereof.

46. The method of claim 35, said activation state being selected from the group consisting of the upregulated and downregulated states.

47. The method of claim 35, said molecule being an antagonist of the on switch control pocket for said kinase.

48. The method of claim 35, said molecule being an agonist of the off switch control pocket for said kinase.

49. The method of claim 35, said method including the step of administering said molecule to an individual undergoing treatment for a condition selected from the group consisting of human inflammation, rheumatoid arthritis, rheumatoid spondylitis, ostero-arthritis, asthma, gouty arthritis, sepsis, septic shock, endotoxic shock, Gram-negative sepsis, toxic shock syndrome, adult respiratory distress syndrome, stroke, reperfusion injury, neural trauma, neural ischemia, psoriasis, restenosis, chronic pulmonary inflammatory disease, bone resorptive diseases, graft-versus-host reaction, Chron's disease, ulcerative colitis, inflammatory bowel disease, pyresis, and combinations thereof.

50. The method of claim 49, said molecule being administered by a method selected from the group consisting of oral, parenteral, inhalation, and subcutaneous.

51. An adduct comprising a molecule binding with a p 38-alpha kinase, said molecule having the formula of claim 34.

52. The adduct of claim 51, said molecule binding at the region of a switch control pocket of said kinase.

53. The adduct of claim 52, said switch control pocket of said kinase comprising an amino acid residue sequence operable for binding to said molecule.

54. The adduct of claim 52, said switch control pocket selected from the group consisting of simple, composite and combined switch control pockets.

55. The adduct of claim 52, said region being selected from the group consisting of the α-C helix, the α-D helix, the catalytic loop, the switch control ligand sequence, the C-terminal residues, the glycine rich loop residues, and combinations thereof.

56. The adduct of claim 55, said α-C helix including SEQ ID NO. 2.

57. The adduct of claim 55, said catalytic loop including SEQ ID NO. 3.

58. The adduct of claim 55, said switch control sequence being selected from the group consisting of SEQ ID NO. 4, SEQ ID NO. 5, and combinations thereof.

59. The adduct of claim 55, said C-lobe residues selected from the group consisting of SEQ ID NO. 6.

60. The adduct of claim 55, said glycine rich loop residues including SEQ ID NO. 7.

61. The adduct of claim 51, said kinase selected from the group consisting of the consensus wild type P 38-alpha kinase sequence and disease polymorphs thereof.

62. A kinase-modulator adduct comprising a p38-alpha kinase having a switch control pocket with a non-naturally occurring molecule bound to the kinase at the region of said switch control pocket, said molecule serving to at least partially regulate the biological activity of said protein by inducing or restricting the conformation of the protein.

63. The adduct of claim 62, said molecule serving to induce a conformation change in said kinase.

64. The adduct of claim 62, said molecule serving to restrict a conformation change in said kinase.

65. The adduct of claim 62, said region of the switch control pocket being selected from the group consisting of the c-C helix, the α-D helix, the catalytic loop, the switch control ligand sequence, the C-terminal residues, the glycine rich loop residues, and combinations thereof.

66. The adduct of claim 65, said α-C helix including SEQ ID NO. 2.

67. The adduct of claim 65, said catalytic loop including SEQ ID NO. 3.

68. The adduct of claim 65, said switch control sequence being selected from the group consisting of SEQ ID NO. 4, SEQ ID NO. 5, and combinations thereof.

69. The adduct of claim 65, said C-lobe residues selected from the group consisting of SEQ ID NO. 6.

70. The adduct of claim 65, said glycine rich loop residues including SEQ ID NO. 7.

71. The adduct of claim 62, said kinase also having a switch control ligand, said ligand interacting in vivo with said pocket to regulate the conformation and biological activity of said kinase such that the kinase will assume a first conformation and a first biological activity upon said ligand-pocket interaction, and will assume a second, different conformation and biological activity in the absence of said ligand-pocket interaction.

72. The adduct of claim 62, said pocket being an on-pocket, said molecule binding with said kinase at the region of said on-pocket as an agonist.

73. The adduct of claim 62, said pocket being an on-pocket, said molecule binding with said kinase at the region of said on-pocket as an antagonist.

74. The adduct of claim 62, said pocket being an off-pocket, said molecule binding with said kinase at the region of said off-pocket as an agonist.

75. The adduct of claim 62, said pocket being an off-pocket, said molecule binding with said kinase at the region of said off-pocket as an antagonist.

76. A method of altering the biological activity of a p38 kinase comprising the steps of:
providing a p38-alpha kinase having a switch control pocket;
contacting said kinase with a non-naturally occurring molecule modulator; and
causing said modulator to bind with said kinase at the region of said pocket in order to at least partially regulate the biological activity of the kinase by inducing or restricting the conformation of the kinase.

77. The method of claim 76, said molecule serving to induce a conformation change in said kinase.

78. The method of claim 76, said molecule serving to restrict a conformation change in said kinase.

79. The method of claim 76, said kinase also having a switch control ligand, said ligand interacting in vivo with said pocket to regulate the conformation and biological activity of said kinase such that the kinase will assume a first conformation and a first biological activity upon said ligand-pocket interaction, and will assume a second, different conformation and biological activity in the absence of said ligand-pocket interaction.

80. The method of claim 76, said pocket being an on-pocket, said molecule binding with said kinase at the region of said on-pocket as an agonist.

81. The method of claim 76, said pocket being an on-pocket, said molecule binding with said kinase at the region of said on-pocket as an antagonist.

82. The method of claim 76, said pocket being an off-pocket, said molecule binding with said kinase at the region of said off-pocket as an agonist.

83. The method of claim 76, said pocket being an off-pocket, said molecule binding with said kinase at the region of said off-pocket as an antagonist.
